(19) United States
(12) Patent Application Publication
(10) Pub. No.: US 2025/0257321 A1
(43) Pub. Date: Aug. 14, 2025

(54) METHODS OF TREATING BRAIN INJURY

(71) Applicant: ASTELLAS INSTITUTE FOR REGENERATIVE MEDICINE, Westborough, MA (US)

(72) Inventors: Erin KIMBREL, Westborough, MA (US); Cesario BORLONGAN, Westborough, MA (US); Daniel Paul POZNIAK, Westborough, MA (US)

(21) Appl. No.: 18/879,342

(22) PCT Filed: Jul. 17, 2023
(86) PCT No.: PCT/US2023/027882

§ 371 Date: Dec. 27, 2024

Related U.S. Application Data
(60) Provisional application No. 63/390,044, filed on Jul. 18, 2022.

(51) Int. Cl. C12N 5/0787; A61K 9/00; A61K 35/15; A61P 25/00; C12N 5/0735; G01N 33/483; G01N 33/50

(52) U.S. Cl. CPC C12N 5/0642; A61K 9/0019; A61K 35/15; A61P 25/00; C12N 5/0606; G01N 33/4833; G01N 33/5005

(57) ABSTRACT

The present invention generally relates to compositions and methods useful for treating a brain injury such as stroke, optic neuropathy, traumatic brain injury, and cerebral palsy. The methods include administering HMCs obtained by in vitro differentiation of pluripotent stem cells and/or extracellular vesicles (EVs) derived from such HMCs (HMC-EVs) into a subject.

### RELATED APPLICATION

This application is a national phase filing under 35 C.F.R. § 371 of and claims priority to PCT Patent Application No. PCT/US2023/027882, filed on Jul. 17, 2023, which claims the benefit of priority to U.S. Provisional Application No. 63/390,044, filed on Jul. 18, 2022, the entire contents of which are incorporated herein by reference.

### FIELD OF THE DISCLOSURE

The instant presently disclosed subject matter relates to methods of treating a brain injury using mesenchymal stem cells and/or extracellular vesicles secreted from the mesenchymal stem cells.

### BACKGROUND OF THE DISCLOSURE

Brain injuries are complex and can have multiple severe clinical outcomes. An acquired brain injury is an injury to the brain that is not hereditary, congenital, degenerative, or induced by birth trauma. The injury results in a change to the brain's neuronal activity, which affects the physical integrity, metabolic activity, or functional ability of nerve cells in the brain. There are two main types of acquired brain injury: traumatic and non-traumatic.

Traumatic brain injury (TBI) is a major cause of death and disability in the United States. More than 1.7 million individuals suffer annually from TBI in US. A TBI is caused by an external force, such as a bump, blow, or jolt to the head that disrupts the normal function of the brain. The severity of a TBI may range from “mild” (i.e., a brief change in mental status or consciousness) to “severe” (i.e., an extended period of unconsciousness or memory loss after the injury). TBIs contribute to about 30% of all injury deaths. (Taylor et al. MMWR Surveill. Summ. 2017; 66(No. SS-9):1-16). Every day, about 153 people in the United States die from injuries that include TBI. Id. Those who survive a TBI can face effects that last a few days, or the rest of their lives. Effects of TBI can include impaired thinking or memory, movement, sensation (e.g., vision or hearing), or emotional functioning (e.g., personality changes, depression).

Approximately 20%-40% of people with TBI experience related vision disorders (Houston K E, et al., Am J Phys. Med. Rehabil. 2017, 96: e70-4). This can include blurred vision, visual field loss, and decreased visual acuity. These symptoms can occur acutely or chronically depending on injury type, location, and severity. TBI can affect diverse parts of the visual system ranging from the optic nerve and tract, lateral geniculate nucleus, and optic radiations, resulting in a variety of visual problems (Barnett B P, el al., Curr Treat Options Neurol., 2015:17:329). One known site of afferent pathway damage is via the optic nerve and tract. Structurally, the optic nerve is vulnerable to compression, traction, crush, laceration, and avulsion injuries. Rapid acceleration, or deceleration, of the head may indirectly lead to optic nerve traction or axonal shearing, which can result in optic neuropathy.

Several treatment options to date for TBI include hyperbaric oxygen therapy, noninvasive brain stimulation, task-oriented functional electrical stimulation, and behavioral therapies (Dang et al. Neural Plasticity 2017; Volume 2017, Article ID 1582182, 6 pages). However, there is still a need for improved treatments for TBI.

Non-traumatic brain injury is usually caused by damage to the brain by internal factors, such as lack of oxygen, exposure to toxins, pressure from tumor, etc. Stroke is an example of non-traumatic brain injury. Stroke is the fifth leading cause of death in the United States, and nearly 800,000 people have a stroke each year. Stroke occurs when a blockage or bleed of the blood vessels either interrupts or reduces the supply of blood to the brain. When this happens, the brain does not receive enough oxygen or nutrients, and brain cells start to die. A person experiencing a stroke needs immediate emergency treatment, such as drugs that break down clots and prevent continued formation of clots. Although strokes can be treatable, some can lead to disability or death.

Cerebral palsy occurs as a result of a brain injury sustained during fetal development or birth. Cerebral palsy is caused by damage to the motor cortex of the brain, which affects muscle control and coordination, including an individual's ability to move, grasp objects, and talk. It is a leading cause of disability in young children and affects about 500,000 children and adults. There is currently no known cure for cerebral palsy.

Nerve and brain cells damaged in brain injuries are generally irreparable because brain tissue cannot regenerate. Stem cell therapies have shown some promise in neuroregenerative treatments. However, there is still a need for improved treatments for brain injuries.

### SUMMARY OF THE DISCLOSURE

The presently disclosed subject matter provides mesenchymal stem cells (MSCs, or also referred to herein as “HMCs”) obtained by in vitro differentiation of pluripotent stem cells, and extracellular vesicles (“EVs”) secreted from the HMCs (HMC-EVs) of the presently disclosed subject matter, and their use in methods of treating brain injuries. Specifically, the inventors of the presently disclosed subject matter have discovered that the HMCs and HMC-EVs of the presently disclosed subject matter are distinct from MSCs and EVs derived from other sources, e.g., adipose tissue-derived MSCs, bone marrow-derived MSCs, and/or umbilical cord blood-derived MSCs. Specifically, the HMCs of the presently disclosed subject matter have a distinct expression profile when compared to other MSCs, e.g., adipose tissue-derived MSCs, bone marrow-derived MSCs, and/or umbilical cord blood-derived MSCs. Proteins/genes that are involved in neuroprotection and cell viability/survival pathways are upregulated in the HMCs of the presently disclosed subject matter, suggesting that the HMCs of the presently disclosed subject matter are able to confer neuroprotective effects, and provide neurotrophic factors, i.e., factors involved in supporting neuronal survival, growth, health and/or recovery. Likewise, the HMC-EVs of the presently disclosed subject matter share a similar profile as the HMCs from which they were secreted. Similar signaling pathways enriched in the HMCs are also enriched in the HMC-EVs when compared to other tissue-derived MSCs and EVs. This distinct profile renders the HMCs and the HMC-EVs to be particularly useful and effective in treating disease, such as brain injuries. Examples of brain injuries treatable with the HMCs and/or HMC-EVs of the presently disclosed subject matter include stroke, traumatic brain injury, acquired brain injury, anoxic brain injury, diffuse axonal brain injury, focal brain injury, subdural hematoma, brain aneurysm, coma, optic neuropathy, and cerebral palsy.

Accordingly, in one aspect, the presently disclosed subject matter provides a method of treating a brain injury in a subject suffering from, or suspected of suffering from, a brain injury, the method comprising administering to the subject an effective amount of EVs secreted from HMCs (HMC-EVs) obtained by in vitro differentiation of pluripotent stem cells, thereby treating the brain injury in the subject.

In some embodiments, the brain injury is selected from the group consisting of stroke, traumatic brain injury, optic neuropathy, cerebral palsy, acquired brain injury, anoxic brain injury, diffuse axonal brain injury, focal brain injury, subdural hematoma, brain aneurysm, and coma. In some embodiments, the brain injury is stroke. In some embodiments, the brain injury is optic neuropathy.

In some embodiments, the method comprises increasing oligodendrocyte and precursor cells in the brain following administration of the HMC-EVs into the subject. In some embodiments, the method comprises preserving myelin in the brain following administration of the HMC-EVs into the subject. In some embodiments, the method comprises preventing oxidative damage in neurons following administration of the HMC-EVs into the subject. In some embodiments, the method comprises preventing neuronal death due to glutamate excitotoxicity injury following administration of the HMC-EVs into the subject. In some embodiments, the method comprises reducing tissue loss in the brain following administration of the EVs into the subject. In some embodiments, the method comprises reducing cell death in the brain following administration of the HMC-EVs into the subject. In some embodiments, the method comprises stimulating pathways involved in the development of neuronal lineage following administration of the HMC-EVs into the subject.

In some embodiments, the HMC-EVs are administered systemically. In some embodiments, the HMC-EVs are administered intracerebrally. In some embodiments, the HMC-EVs are administered intrathecally. In some embodiments, the HMC-EVs are administered intracisternally. In some embodiments, the HMC-EVs are administered intraperitoneally.

In some embodiments, the subject is a human.

In some embodiments, the HMCs are obtained by in vitro differentiation of human pluripotent stem cells. In some embodiments, the pluripotent stem cells are further differentiated into hemangioblasts. In some embodiments, the pluripotent stem cells are embryonic stem cells. In some embodiments, the pluripotent stem cells are induced pluripotent stem cells. In some embodiments, the induced pluripotent stem cells are produced by contacting a cell with one or more reprogramming factors.

In some embodiments, the HMC-EVs express at least one of the miRNA in Table 9 at a higher level compared to EVs secreted from umbilical cord blood-derived MSCs (UCB-MSC-EVs).

In some embodiments, the HMC-EVs express at least one of the miRNA in Table 10 at a lower level compared to UCB-MSC-EVs.

In some embodiments, the HMC-EVs express at least one of the miRNA in Table 11 at a higher level compared to EVs secreted from bone marrow-derived MSCs (BM-MSC-EVs).

In some embodiments, the HMC-EVs express at least one of the miRNA in Table 12 at a lower level compared to BM-MSC-EVs.

In some embodiments, the HMC-EVs express at least one of the miRNA in Table 13 at a higher level compared to EVs secreted from adipose tissue-derived MSCs (AD-MSC-EVs).

In some embodiments, the HMC-EVs express at least one of the miRNA in Table 14 at a lower level compared to AD-MSC-EVs).

In some embodiments, the HMC-EVs express at least one of the proteins in Table 15 at a higher level compared to UCB-MSC-EVs.

In some embodiments, the HMC-EVs express at least one of the proteins in Table 16 at a lower level compared to UCB-MSC-EVs.

In some embodiments, the HMC-EVs express at least one of the proteins in Table 17 at a higher level compared to BM-MSC-EVs.

In some embodiments, the HMC-EVs express at least one of the proteins in Table 18 at a lower level compared to BM-MSC-EVs.

In some embodiments, the HMC-EVs express at least one of the proteins in Table 19 at a higher level compared to AD-MSC-EVs.

In some embodiments, the HMC-EVs express at least one of the proteins in Table 20 at a lower level compared to AD-MSC-EVs.

In some embodiments, the HMC-EVs express at least one of the miRNAs selected from the group consisting of hsa-miR-125b-5p, hsa-miR-181a-5p, hsa-miR-199b-5p, hsa-miR-21-5p, hsa-miR-23a-3p, hsa-miR-125a-5p, hsa-miR-106a-5p+hsa-miR-17-5p and hsa-miR-221-3p at a higher level compared to BM-MSC-EVs, UCB-MSC-EVs, and/or AD-MSC-EVs.

In some embodiments, the HMC-EVs express at least one of the proteins selected from the group consisting of ALDOC, ANXA5, APBB2, BASP1, CAV1, CD81, CD99, CKM, EPB41L3, FDPS, GNAQ, GNG12, GP9, H2AC20, H2AC21, H3-3A, H3-7, H4-16, HLA-A, ITGA2, KPNA2, KRAS, KRT4, LRRC59, MAMDC2, MARCKSL1, MDGA1, MERTK, MFGE8, MMP14, MVP, PCDH1, PDGFRB, PDIA3, RPL13, RPS18, RPS3A, RPS4X, SDCBP, SLC2A1, SLC3A2, TAGLN2, TNC, TSPAN14, TSPAN33, TSPAN9, TTYH3, UCHL1, VAT1, YWHAB, and YWHAQ at a higher level compared to BM-MSC-EVs, UCB-MSC-EVs, and/or AD-MSC-EVs.

In some embodiments, the HMC-EVs express at least one of the proteins selected from the group consisting of ADGRG6, AGRN, ANXA6, APOC4, ARHGAP1, ARGHDIA, ARL8A, ARPC5, B2M, BBS1, BLVRA, BST1, CA2, CCN2, CCNB3, CD34, CD36, CD47, CORO1A, DTD1, EEF1D, EEF1G, ENG, ESD, GNAI2, GNB1, H1-3, H2BC15, HIP1, KIF11, LAMP1, LAP3, LGALS1, LTBP3, MAPK3, MARCKS, MBTD1, MDH1, MOB1B, MYL12B, MYO1F, MYO3A, NIBAN2, PEBP1, PF4, PGAP1, PLOD1, PPP2R1A, PRSS23, PXDN, RALA, RAP2A, RPS13, RPS3, RPSA, S100A11, SLC44A1, SLC44A2, SLTM, SMG1, SPARC, SRSF8, STRADB, STX11, STXBP2, TGM2, TPP1, TPTE2, TRIM5, TRPM2, TUBA8, TUBB3, VCAN, YWHAE, and ZFN607 at a higher level compared to BM-MSC-EVs, UCB-MSC-EVs, and/or AD-MSC-EVs.

In some embodiments, the HMC-EVs express at least one of the proteins selected from the group consisting of ADIPOQ, CAT, CEP290, IGLV6-57, TAS2R33, and TMEM198 at a lower level compared to EVs secreted from BM-MSC-EVs, UCB-MSC-EVs, and/or AD-MSC-EVs.

In some embodiments, the HMC-EVs express at least one of the proteins selected from the group consisting of AKAP9, ALB, ALOX5, APLP2, CD109, CDSN, CHST9, ERC1, F11, ARMCX5, LAMB4, LRRTM2, LTF, MSH6, OAF, OLFML3, PAK6, RGS14, SEMA7A, SURF1, and TRIM4 at a lower level compared to BM-MSC-EVs, UCB-MSC-EVs, and/or AD-MSC-EVs.

In some embodiments, the HMC-EVs express at least one of the miRNA in Table 21 at a higher level compared to the HMCs.

In some embodiments, the HMC-EVs express at least one of the miRNA in Table 22 at a lower level compared to the HMCs.

In some embodiments, about 1×10⁶ to about 1×10¹³ HMC-EVs are administered to the subject. In some embodiments, about 10×10¹⁰ or about 30×10¹⁰ HMC-EVs are administered to the subject.

In some embodiments, the HMC-EVs are administered in a pharmaceutical composition.

In some embodiments, the pharmaceutical composition comprises (a) a buffer, maintaining the solution at a physiological pH; (b) at least 2 mM or at least 0.05% (w/v) glucose; and (c) an osmotically active agent maintaining the solution at a physiological osmolarity.

In some embodiments, the glucose is D-glucose (Dextrose). In some embodiments, the osmotically active agent is a salt. In some embodiments, the osmotically active agent is a magnesium salt, phosphate salt, sulfate salt, chloride salt, poorly absorbed disaccharides, such as lactulose, sugar alcohols, such as mannitol and sorbitol, and polyethylene glycol, or a combination thereof. In some embodiments, the osmotically active agent is CaCl2), KCl, NaCl, KH2PO4, Na3HPO4, MgCl2, MgSO4, HEPES, NaHCO₃, or a combination thereof. In some embodiments, the salt is sodium chloride.

In some embodiments, the method further comprises administering to the subject an effective amount of HMCs obtained by in vitro differentiation of pluripotent stem cells.

In one aspect, the presently disclosed subject matter provides a method of treating a brain injury in a subject suffering from, or suspected of suffering from, a brain injury, the method comprising administering to the subject an effective amount of HMCs obtained by in vitro differentiation of pluripotent stem cells, thereby treating the brain injury in the subject.

In some embodiments, the brain injury is selected from the group consisting of stroke, traumatic brain injury, cerebral palsy, acquired brain injury, anoxic brain injury, diffuse axonal brain injury, focal brain injury, subdural hematoma, brain aneurysm, optic neuropathy, and coma.

In some embodiments, the brain injury is stroke.

In some embodiments, the brain injury is optic neuropathy.

In some embodiments, the method comprises preserving myelin in the brain following administration of the HMCs into the subject. In some embodiments, the method comprises suppressing neuroinflammatory responses following administration of the HMCs into the subject. In some embodiments, the method comprises reducing microglial and astrocyte activation in the brain following administration of the HMCs into the subject. In some embodiments, the method comprises stimulating pathways involved in cell survival following administration of the HMCs into the subject. In some embodiments, the method comprises stimulating expression of a neuroprotective gene in the brain following administration of the HMCs into the subject. In some embodiments, the neuroprotective gene is selected from the group consisting of heat shock protein family B member 1 (HSPB1), insulin-like growth factor 1 (IGF2), and secreted phosphoprotein 1 (SPP1). In some embodiments, the method comprises stimulating pathways involved in synaptic transmission in the brain following administration of the HMCs into the subject. In some embodiments, the method comprises stimulating pathways involved in the development of neuronal lineage following administration of the HMCs into the subject. In some embodiments, the method comprises reducing apoptosis following administration of the HMCs into the subject.

In some embodiments, the brain injury is traumatic brain injury.

In some embodiments, the method comprises reducing tissue loss in the brain following administration of the HMCs into the subject. In some embodiments, the method comprises reducing cell death in the brain following administration of the HMCs into the subject. In some embodiments, the method comprises increasing neurogenesis following the administration of the HMCs into the subject. In some embodiments, the method comprises reducing the presence of microglia and macrophages in the cortex and striatum following the administration of the HMCs into the subject. In some embodiments, the method comprises reducing inflammation of the spleen following the administration of the HMCs into the subject. In some embodiments, the method comprises migration of HMCs across the blood-brain barrier to the cortex, striatum, and/or hippocampus.

In some embodiments, the brain injury is cerebral palsy.

In some embodiments, the method comprises reducing apoptosis in the brain following administration of the HMCs into the subject. In some embodiments, the method comprises reducing lesion size in the brain following administration of the HMCs into the subject. In some embodiments, the method comprises reducing microglial and astrocyte activation in the brain following administration of the HMCs into the subject. In some embodiments, the method comprises preserving myelin of the corpus callosum following administration of the HMCs into the subject. In some embodiments, the method comprises at least a partial rescue of Olig2 in the brain following administration of the HMCs into the subject.

In some embodiments, the HMCs are administered systemically. In some embodiments, the HMCs are administered intracerebrally. In some embodiments, the HMCs are administered intrathecally. In some embodiments, the HMCs are administered intracisternally. In some embodiments, the HMCs are administered intraperitoneally. In some embodiments, the mesenchymal stem cells are human cells.

In some embodiments, the subject is a human.

In some embodiments, the pluripotent stem cells are further differentiated into hemangioblasts. In some embodiments, the pluripotent stem cells are embryonic stem cells. In some embodiments, the pluripotent stem cells are induced pluripotent stem cells. In some embodiments, the pluripotent stem cells are human pluripotent stem cells.

In some embodiments, the HMCs have been passaged no more than 5 times in vitro before administration into the subject.

In some embodiments, the HMCs express at least one of the genes in Table 3 at a higher level compared to bone marrow-derived MSCs (BM-MSCs).

In some embodiments, the HMCs express at least one of the genes in Table 4 at a lower level compared to BM-MSCs.

In some embodiments, the HMCs express at least one of the genes in Table 5 at a higher level compared to umbilical cord blood-derived MSCs (UCB-MSCs).

In some embodiments, the HMCs express at least one of the genes in Table 6 at a lower level compared to UCB-MSCs.

In some embodiments, the HMCs express at least one of the genes in Table 7 at a higher level compared to adipose tissue-derived MSCs (AD-MSCs).

In some embodiments, the HMCs express at least one of the genes in Table 8 at a lower level compared to AD-MSCs.

In some embodiments, the HMCs express, in a basal state, mRNA encoding interleukin-6 (IL-6) at a level less than ten percent of the IL-6 mRNA level expressed by BM-MSCs, in a basal state, and wherein the HMCs express, in a basal state, mRNA encoding CD24 at a level that is greater than the CD24 mRNA level expressed by BM-MSCs in a basal state.

In some embodiments, the HMCs express at least one of the genes selected from the group consisting of CALR, UBB, PKM, CXCL8, C15orf48, PSME2, TPM3, ANKRD1, PFN1, SRGN, ACTB, MDK, TAGLN2, CFL1, HSP90AA1, HSPA8, CXCL12, UCHL1, HMGA2, HMGA1, HN1, PTMA, SP90AB1, PRDX1, GSTP1, KRT18, IGFBP4, CALD1, COL4A1, COL4A2, and GAPDH at a higher level compared to adipose tissue-derived MSCs (AD-MSCs).

In some embodiments, the HMCs express at least one of the genes selected from the group consisting of TMSB4X, ACTG1, GSTP1, KRT18, IGFBP5, NPY, KRT8, PRDX6, MDK, DKK3, UCHL1, TUBB3, HN1, PTMA, HSP90AB1, HMGA1, HSPA8, TAGLN2, ANKRD1, PFN1, CYBA, and UBB at a higher level compared to AD-MSCs.

In some embodiments, the HMCs express at least one of the genes selected from the group consisting of SERPINE1, ACTA2, TPM2, CTGF, SERPINE2, CRYAB, ELN, MFGE8, ANXA2, POSTN, VIM, MFAP5, ISLR, THBS1, TIMP3, DKK1, COL6A3, COL6A1, TPT1, BCYRN1, COL1A1, SPARC, TPM1, BGN, COL1A2, COL3A1, TGFBI, CRLF1, COMP, NEAT1, MT-CO3, MT-CO2, MT-ATP8, MT-CYB, MT-CO1, MT-ATP6, MT-ND4, MT-ND4L, MT-ND5, MT-ND6, MT-ND3, MT-ND1, MT-ND2, GREM1, TMSB4X, ITGB1, LMNA, H2AFZ, FTL, EEF1G, NPM1, EEF1A1, RACK1, ACTG1, and TPM4 at a lower level compared to AD-MSCs.

In some embodiments, the HMCs express at least one of the genes selected from the group consisting of SERPINE1, S100A6, CD59, POSTN, VIM, MFAP5, ISLR, THBS1, COL6A3, TIMP3, ELN, ANXA2, COL1A1, BCYRN1, CCDC80, COL6A1, COL6A2, BGN, COL1A2, COL3A1, TGFB1, CRLF1, COMP, and GREM1 at a lower level compared to AD-MSCs.

In some embodiments, the HMCs express at least one of the genes selected from the group consisting of MT1X, MT1G, TMSB10, CCL8, INHBA, CTSB, SERPINB2, ADM, APOL1, FTH1, CCL2, CCL5, CSF1, IL1B, IGFBP3, P4HB, DCN, FSTL1, ANXA5, LOX, CD63, CTSZ, FN1, LGALS1, LDHA, RCN3, MMP2, and TIMP1 at a lower level compared to AD-MSCs.

In some embodiments, the HMCs express at least one of the genes selected from the group consisting of PPIA, NPM1, HNRNPA1, IGFBP5, KRT19, KRT18, GSTP1, TUBB, TUBA1B, KRT8, HN1, PTMA, TUBA1C, HSPA8, HMGA1, CFL1, MYL6, ACTB, UCHL1, TAGLN2, MDK, GREM1, MMP1, and CTSC at a higher level compared to bone marrow-derived MSCs (BM-MSCs).

In some embodiments, the HMCs express at least one of the genes selected from the group consisting of ANXA2, TPT1, VIM, COL6A1, BGN, COL6A2, CTGF, TIMP3, ACTA2, COL3A1, SPARC, ITGB1, SERPINH1, TPM2, TGFBI, COL1A1, TPM1, COL6A3, TPM4, SERPINE2, CALD1, COL1A2, TAGLN, MYL9, MT-RNR2, POSTN at a lower level compared to BM-MSCs.

In some embodiments, the HMCs express at least one of the miRNA in Table 21 at a lower level compared to the HMC-EVs secreted from the HMCs.

In some embodiments, the HMCs express at least one of the miRNA in Table 22 at a higher level compared to the HMC-EVs secreted from the HMCs.

In some embodiments, about 1×10⁶ to about 1×10¹³ HMCs are administered to the subject.

In some embodiments, the HMCs are administered in a pharmaceutical composition.

In some embodiments, the pharmaceutical composition comprises (a) a buffer, maintaining the solution at a physiological pH; (b) at least 2 mM or at least 0.05% (w/v) glucose; and (c) an osmotically active agent maintaining the solution at a physiological osmolarity.

In some embodiments, the glucose is D-glucose (Dextrose). In some embodiments, the osmotically active agent is a salt. In some embodiments, the salt is sodium chloride.

In another aspect, the presently disclosed subject matter provides a method of treating a brain injury in a subject suffering from, or suspected of suffering from, a brain injury, the method comprising administering to the subject an effective amount of EVs secreted from HMCs obtained by in vitro differentiation of pluripotent stem cells, and an effective amount of HMCs obtained by in vitro differentiation of pluripotent stem cells, thereby treating the brain injury in the subject.

In one aspect, the presently disclosed subject matter provides a composition comprising HMCs obtained by in vitro differentiation of pluripotent stem cells, wherein the HMCs express at least one of the genes selected from the group consisting of CALR, UBB, PKM, CXCL8, C15orf48, PSME2, TPM3, ANKRD1, PFN1, SRGN, ACTB, MDK, TAGLN2, CFL1, HSP90AA1, HSPA8, CXCL12, UCHL1, HMGA2, HMGA1, HN1, PTMA, SP90AB1, PRDX1, GSTP1, KRT18, IGFBP4, CALD1, COL4A1, COL4A2, and GAPDH at a higher level compared to AD-MSCs.

In one aspect, the presently disclosed subject matter provides a composition comprising HMCs obtained by in vitro differentiation of pluripotent stem cells, wherein the HMCs express at least one of the genes selected from the group consisting of TMSB4X, ACTG1, GSTP1, KRT18, IGFBP5, NPY, KRT8, PRDX6, MDK, DKK3, UCHL1, TUBB3, HN1, PTMA, HSP90AB1, HMGA1, HSPA8, TAGLN2, ANKRD1, PFN1, CYBA, and UBB at a higher level compared to AD-MSCs.

In one aspect, the presently disclosed subject matter provides a composition comprising HMCs obtained by in vitro differentiation of pluripotent stem cells, wherein the HMCs express at least one of the genes selected from the group consisting of PPIA, NPM1, HNRNPA1, IGFBP5, KRT19, KRT18, GSTP1, TUBB, TUBA1B, KRT8, HN1, PTMA, TUBA1C, HSPA8, HMGA1, CFL1, MYL6, ACTB, UCHL1, TAGLN2, MDK, GREM1, MMP1, and CTSC at a higher level compared to BM-MSCs.

In one aspect, the presently disclosed subject matter provides a composition comprising HMCs obtained by in vitro differentiation of pluripotent stem cells, wherein the HMCs express at least one of the genes selected from the group consisting of SERPINE1, ACTA2, TPM2, CTGF, SERPINE2, CRYAB, ELN, MFGE8, ANXA2, POSTN, VIM, MFAP5, ISLR, THBS1, TIMP3, DKK1, COL6A3, COL6A1, TPT1, BCYRN1, COL1A1, SPARC, TPM1, BGN, COL1A2, COL3A1, TGFBI, CRLF1, COMP, NEAT1, MT-CO3, MT-CO2, MT-ATP8, MT-CYB, MT-CO1, MT-ATP6, MT-ND4, MT-ND4L, MT-ND5, MT-ND6, MT-ND3, MT-ND1, MT-ND2, GREM1, TMSB4X, ITGB1, LMNA, H2AFZ, FTL, EEF1G, NPM1, EEF1A1, RACK1, ACTG1, and TPM4 at a lower level compared to AD-MSCs.

In one aspect, the presently disclosed subject matter provides a composition comprising HMCs obtained by in vitro differentiation of pluripotent stem cells, wherein the HMCs express at least one of the genes selected from the group consisting of SERPINE1, S100A6, CD59, POSTN, VIM, MFAP5, ISLR, THBS1, COL6A3, TIMP3, ELN, ANXA2, COL1A1, BCYRN1, CCDC80, COL6A1, COL6A2, BGN, COL1A2, COL3A1, TGFB1, CRLF1, COMP, and GREM1 at a lower level compared to AD-MSCs.

In one aspect, the presently disclosed subject matter provides a composition comprising HMCs obtained by in vitro differentiation of pluripotent stem cells, wherein the HMCs express at least one of the genes selected from the group consisting of MT1X, MT1G, TMSB10, CCL8, INHBA, CTSB, SERPINB2, ADM, APOL1, FTH1, CCL2, CCL5, CSF1, IL1B, IGFBP3, P4HB, DCN, FSTL1, ANXA5, LOX, CD63, CTSZ, FN1, LGALS1, LDHA, RCN3, MMP2, and TIMP1 at a lower level compared to AD-MSCs.

In one aspect, the presently disclosed subject matter provides a composition comprising HMCs obtained by in vitro differentiation of pluripotent stem cells, wherein the HMCs express at least one of the genes selected from the group consisting of ANXA2, TPT1, VIM, COL6A1, BGN, COL6A2, CTGF, TIMP3, ACTA2, COL3A1, SPARC, ITGB1, SERPINH1, TPM2, TGFBI, COL1A1, TPM1, COL6A3, TPM4, SERPINE2, CALD1, COL1A2, TAGLN, MYL9, MT-RNR2, POSTN at a lower level compared to BM-MSCs.

In some embodiments, the HMCs further express at least one of the genes in Table 3 at a higher level compared to BM-MSCs.

In some embodiments, the HMCs further express at least one of the genes in Table 4 at a lower level compared to BM-MSCs.

In some embodiments, the HMCs further express at least one of the genes in Table 5 at a higher level compared to UCB-MSCs.

In some embodiments, the HMCs further express at least one of the genes in Table 6 at a lower level compared to UCB-MSCs.

In some embodiments, the HMCs further express at least one of the genes in Table 7 at a higher level compared to AD-MSCs.

In some embodiments, the HMCs further express at least one of the genes in Table 8 at a lower level compared to AD-MSCs.

In one aspect, the presently disclosed subject matter provides a pharmaceutical composition comprising the HMCs of the presently disclosed subject matter, and a pharmaceutically acceptable carrier.

In one aspect, the presently disclosed subject matter provides a population of HMC-EVs of the presently disclosed subject matter.

In some embodiments, the HMC-EVs express at least one of the miRNA in Table 9 at a higher level compared to UCB-MSC-EVs.

In some embodiments, the HMC-EVs express at least one of the miRNA in Table 10 at a lower level compared to UCB-MSC-EVs.

In some embodiments, the HMC-EVs express at least one of the miRNA in Table 11 at a higher level compared to BM-MSC-EVs.

In some embodiments, the HMC-EVs express at least one of the miRNA in Table 12 at a lower level compared to BM-MSC-EVs.

In some embodiments, the HMC-EVs express at least one of the miRNA in Table 13 at a higher level compared to AD-MSC-EVs.

In some embodiments, the HMC-EVs express at least one of the miRNA in Table 14 at a lower level compared to AD-MSC-EVs.

In some embodiments, the HMC-EVs express at least one of the proteins in Table 15 at a higher level compared to UCB-MSC-EVs.

In some embodiments, the HMC-EVs express at least one of the proteins in Table 16 at a lower level compared to UCB-MSC-EVs.

In some embodiments, the HMC-EVs express at least one of the proteins in Table 17 at a higher level compared to BM-MSC-EVs.

In some embodiments, the HMC-EVs express at least one of the proteins in Table 18 at a lower level compared to BM-MSC-EVs.

In some embodiments, the HMC-EVs express at least one of the proteins in Table 19 at a higher level compared to AD-MSC-EVs.

In some embodiments, the HMC-EVs express at least one of the proteins in Table 20 at a lower level compared to AD-MSC-EVs.

In some embodiments, the HMC-EVs express at least one of the miRNA in Table 21 at a higher level compared to the HMCs.

In some embodiments, the HMC-EVs express at least one of the miRNA in Table 22 at a lower level compared to the HMCs.

In some embodiments, the HMC-EVs express at least one of the miRNAs selected from the group consisting of hsa-miR-125b-5p, hsa-miR-181a-5p, hsa-miR-199b-5p, hsa-miR-21-5p, hsa-miR-23a-3p, hsa-miR-125a-5p, hsa-miR-106a-5p+hsa-miR-17-5p and hsa-miR-221-3p at a higher level compared to EVs secreted from BM-MSC-EVs, UCB-MSC-EVs, and/or AD-MSC-EVs.

In some embodiments, the HMC-EVs express at least one of the proteins selected from the group consisting of ALDOC, ANXA5, APBB2, BASP1, CAV1, CD81, CD99, CKM, EPB41L3, FDPS, GNAQ, GNG12, GP9, H2AC20, H2AC21, H3-3A, H3-7, H4-16, HLA-A, ITGA2, KPNA2, KRAS, KRT4, LRRC59, MAMDC2, MARCKSL1, MDGA1, MERTK, MFGE8, MMP14, MVP, PCDH1, PDGFRB, PDIA3, RPL13, RPS18, RPS3A, RPS4X, SDCBP, SLC2A1, SLC3A2, TAGLN2, TNC, TSPAN14, TSPAN33, TSPAN9, TTYH3, UCHL1, VAT1, YWHAB, and YWHAQ at a higher level compared to BM-MSC-EVs, UCB-MSC-EVs, and/or AD-MSC-EVs.

In some embodiments, the HMC-EVs express at least one of the proteins selected from the group consisting of ADGRG6, AGRN, ANXA6, APOC4, ARHGAP1, ARGHDIA, ARL8A, ARPC5, B2M, BBS1, BLVRA, BST1, CA2, CCN2, CCNB3, CD34, CD36, CD47, CORO1A, DTD1, EEF1D, EEF1G, ENG, ESD, GNAI2, GNB1, H1-3, H2BC15, HIP1, KIF11, LAMP1, LAP3, LGALS1, LTBP3, MAPK3, MARCKS, MBTD1, MDH1, MOB1B, MYL12B, MYO1F, MYO3A, NIBAN2, PEBP1, PF4, PGAP1, PLOD1, PPP2RIA, PRSS23, PXDN, RALA, RAP2A, RPS13, RPS3, RPSA, S100A11, SLC44A1, SLC44A2, SLTM, SMG1, SPARC, SRSF8, STRADB, STX11, STXBP2, TGM2, TPP1, TPTE2, TRIM5, TRPM2, TUBA8, TUBB3, VCAN, YWHAE, and ZFN607 at a higher level compared to BM-MSC-EVs, UCB-MSC-EVs, and/or AD-MSC-EVs.

In some embodiments, the HMC-EVs express at least one of the proteins selected from the group consisting of ADIPOQ, CAT, CEP290, IGLV6-57, TAS2R33, and TMEM198 at a lower level compared to BM-MSC-EVs, UCB-MSC-EVs, and/or AD-MSC-EVs.

In some embodiments, the HMC-EVs express at least one of the proteins selected from the group consisting of AKAP9, ALB, ALOX5, APLP2, CD109, CDSN, CHST9, ERC1, F11, ARMCX5, LAMB4, LRRTM2, LTF, MSH6, OAF, OLFML3, PAK6, RGS14, SEMA7A, SURF1, and TRIM4 at a lower level compared to BM-MSC-EVs, UCB-MSC-EVs, and/or AD-MSC-EVs.

In one aspect, the presently disclosed subject matter provides a pharmaceutical composition comprising the HMC-EVs of the presently disclosed subject matter, and a pharmaceutically acceptable carrier.

In one aspect, the presently disclosed subject matter provides a population of HMC-EVs secreted from HMCs obtained by in vitro differentiation of pluripotent stem cells, wherein the HMC-EVs express at least one of the miRNA in Table 9 at a higher level compared to UCB-MSC-EVs.

In one aspect, the presently disclosed subject matter provides a population of HMC-EVs secreted from HMCs obtained by in vitro differentiation of pluripotent stem cells, wherein the HMC-EVs express at least one of the miRNA in Table 10 at a lower level compared to UCB-MSC-EVs.

In one aspect, the presently disclosed subject matter provides a population of HMC-EVs secreted from HMCs obtained by in vitro differentiation of pluripotent stem cells, wherein the HMC-EVs express at least one of the miRNA in Table 11 at a higher level compared to BM-MSC-EVs.

In one aspect, the presently disclosed subject matter provides a population of HMC-EVs secreted from HMCs obtained by in vitro differentiation of pluripotent stem cells, wherein the HMC-EVs express at least one of the miRNA in Table 12 at a lower level compared to BM-MSC-EVs.

In one aspect, the presently disclosed subject matter provides a population of HMC-EVs secreted from HMCs obtained by in vitro differentiation of pluripotent stem cells, wherein the HMC-EVs express at least one of the miRNA in Table 13 at a higher level compared to AD-MSC-EVs.

In one aspect, the presently disclosed subject matter provides a population of HMC-EVs secreted from HMCs obtained by in vitro differentiation of pluripotent stem cells, wherein the HMC-EVs express at least one of the miRNA in Table 14 at a lower level compared to EVs secreted from AD-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins in Table 15 at a higher level compared to UCB-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins in Table 16 at a lower level compared to UCB-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins in Table 17 at a higher level compared to BM-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins in Table 18 at a lower level compared to BM-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins in Table 19 at a higher level compared to AD-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins in Table 20 at a lower level compared to AD-MSC-EVs.

In one aspect, the presently disclosed subject matter provides a population of HMC-EVs secreted from HMCs obtained by in vitro differentiation of pluripotent stem cells, wherein the HMC-EVs express at least one of the miRNA in Table 21 at a higher level compared to the HMCs.

In one aspect, the presently disclosed subject matter provides a population of HMC-EVs secreted from HMCs obtained by in vitro differentiation of pluripotent stem cells, wherein the HMC-EVs express at least one of the miRNA in Table 22 at a lower level compared to the HMCs.

In one aspect, the presently disclosed subject matter provides a population of HMC-EVs secreted from HMCs obtained by in vitro differentiation of pluripotent stem cells, wherein the HMC-EVs express at least one of the miRNAs selected from the group consisting of hsa-miR-125b-5p, hsa-miR-181a-5p, hsa-miR-199b-5p, hsa-miR-21-5p, hsa-miR-23a-3p, hsa-miR-125a-5p, hsa-miR-106a-5p+hsa-miR-17-5p and hsa-miR-221-3p at a higher level compared to BM-MSC-EVs, UCB-MSC-EVs, and/or AD-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins selected from the group consisting of ALDOC, ANXA5, APBB2, BASP1, CAV1, CD81, CD99, CKM, EPB41L3, FDPS, GNAQ, GNG12, GP9, H2AC20, H2AC21, H3-3A, H3-7, H4-16, HLA-A, ITGA2, KPNA2, KRAS, KRT4, LRRC59, MAMDC2, MARCKSL1, MDGA1, MERTK, MFGE8, MMP14, MVP, PCDH1, PDGFRB, PDIA3, RPL13, RPS18, RPS3A, RPS4X, SDCBP, SLC2A1, SLC3A2, TAGLN2, TNC, TSPAN14, TSPAN33, TSPAN9, TTYH3, UCHL1, VAT1, YWHAB, and YWHAQ at a higher level compared to BM-MSC-EVs, UCB-MSC-EVs, and/or AD-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins selected from the group consisting of ADGRG6, AGRN, ANXA6, APOC4, ARHGAP1, ARGHDIA, ARL8A, ARPC5, B2M, BBS1, BLVRA, BST1, CA2, CCN2, CCNB3, CD34, CD36, CD47, CORO1A, DTD1, EEF1D, EEF1G, ENG, ESD, GNAI2, GNB1, H1-3, H2BC15, HIP1, KIF 11, LAMP1, LAP3, LGALS1, LTBP3, MAPK3, MARCKS, MBTD1, MDH1, MOB1B, MYL12B, MYO1F, MYO3A, NIBAN2, PEBP1, PF4, PGAP1, PLOD1, PPP2RIA, PRSS23, PXDN, RALA, RAP2A, RPS13, RPS3, RPSA, S100A11, SLC44A1, SLC44A2, SLTM, SMG1, SPARC, SRSF8, STRADB, STX11, STXBP2, TGM2, TPP1, TPTE2, TRIM5, TRPM2, TUBA8, TUBB3, VCAN, YWHAE, and ZFN607 at a higher level compared to BM-MSC-EVs, UCB-MSC-EVs, and/or AD-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins selected from the group consisting of ADIPOQ, CAT, CEP290, IGLV6-57, TAS2R33, and TMEM198 at a lower level compared to BM-MSC-EVs, UCB-MSC-EVs, and/or AD-MSC-EVs.

In one aspect, the presently disclosed subject matter provides a population of HMC-EVs secreted from HMCs obtained by in vitro differentiation of pluripotent stem cells, wherein the HMC-EVs express at least one of the proteins selected from the group consisting of AKAP9, ALB, ALOX5, APLP2, CD109, CDSN, CHST9, ERC1, F11, ARMCX5, LAMB4, LRRTM2, LTF, MSH6, OAF, OLFML3, PAK6, RGS14, SEMA7A, SURF1, and TRIM4 at a lower level compared to BM-MSC-EVs, UCB-MSC-EVs, and/or AD-MSC-EVs.

In one aspect, the presently disclosed subject matter provides a pharmaceutical composition comprising the HMC-EVs of the presently disclosed subject matter, and a pharmaceutically acceptable carrier.

The presently disclosed subject matter also provides a method of determining neurite outgrowth of an HMC and/or HMC-EV population. The method comprises (a) preparing a mixed neuronal culture from an isolated cerebral cortex, (b) plating the HMC and/or HMC-EV population on a permeable membrane, (c) applying strain on the mixed neuronal culture, (d) overlaying the strained mixed neuronal culture with the permeable membrane of step (b), and (e) measuring neurite outgrowth of the mixed neuronal culture. In an embodiment, step (d) is cultured in a media substantially lacking in serum. In another embodiment, the method further comprises determining gene expression of the mixed neuronal culture in the presence and absence of the HMC and/or HMC-EV population. In another embodiment, the strain is a physical scratch made in the mixed neuronal culture. In another embodiment, the strain is vacuum pressure and positive air pressure applied to the mixed neuronal culture. In another embodiment, the strain may be applied at 15% to 0% stretching oscillations.

The presently disclosed subject matter also provides a method of determining neurite outgrowth of an HMC and/or HMC-EV population. The method comprises preparing a mixed neuronal culture from an isolated cerebral cortex, (b) plating the HMC and/or HMC-EV population on a permeable membrane, (c) applying strain on the mixed neuronal culture, (d) overlaying the strained mixed neuronal culture with the permeable membrane of step (b), and (e) measuring neurite outgrowth of the mixed neuronal culture. In an embodiment, the method further comprises determining gene expression of the mixed neuronal culture in the presence and absence of the HMC and/or HMC-EV population. In another embodiment, the strain is a physical scratch made in the mixed neuronal culture. In another embodiment, the strain is vacuum pressure and positive air pressure applied to the mixed neuronal culture. In another embodiment, the strain is applied at 15% to 0% stretching oscillations.

### BRIEF DESCRIPTION OF THE DRAWINGS

FIG. 1. shows results of the elevated body swing test (EBST) in rats induced with TBI by controlled cortical impact (CCI) and administered with HMCs or vehicle intracerebrally (IC) or intravenously (IV).

FIG. 2 shows forelimb akinesia in rats induced with TBI by controlled cortical impact (CCI) and administered with HMCs or vehicle intracerebrally (IC) or intravenously (IV).

FIG. 3 shows paw grasp in rats induced with TBI by controlled cortical impact (CCI) and administered with HMCs or vehicle intracerebrally (IC) or intravenously (IV).

FIG. 4A shows H&E staining of the brains of rats induced with TBI by controlled cortical impact (CCI) and administered with HMCs or vehicle intracerebrally (IC) or intravenously (IV). FIG. 4B shows a bar graph of the TBI impact area in the rats as measured by H&E staining.

FIG. 5A shows Nissl staining of the peri-impact cortex of rats induced with TBI by controlled cortical impact (CCI) and administered with HMCs or vehicle intracerebrally (IC) or intravenously (IV). FIG. 5B shows a bar graph of the percentage of live cells in the peri-impact cortex of the rats as determined by Nissl staining. FIG. 5C shows Nissl staining of the striatum in the rats induced with TBI by controlled cortical impact (CCI) and administered with HMCs or vehicle intracerebrally (IC) or intravenously (IV). FIG. 5D shows a bar graph of the percentage of live cells in the striatum of the rats as determined by Nissl staining. FIG. 5E shows Nissl staining of the hippocampus of rats induced with TBI by controlled cortical impact (CCI) and administered with HMCs or vehicle intracerebrally (IC) or intravenously (IV). FIG. 5F shows a bar graph of the percentage of live cells in the hippocampus of the rats as determined by Nissl staining.

FIG. 6A shows doublecortin (DCX) staining of the cortex of rats induced with TBI by controlled cortical impact (CCI) and administered with HMCs or vehicle intracerebrally (IC) or intravenously (IV). FIG. 6B shows a bar graph of the DCX cell count in the cortex area of the rats. FIG. 6C shows DCX staining of the striatum of rats induced with TBI by controlled cortical impact (CCI) and administered with HMCs or vehicle intracerebrally (IC) or intravenously (IV). FIG. 6D shows a bar graph of the DCX cell count in the striatum area of the rats. FIG. 6E shows DCX staining of the hippocampus of rats induced with TBI by controlled cortical impact (CCI) and administered with HMCs or vehicle intracerebrally (IC) or intravenously (IV). FIG. 6F shows a bar graph of the DCX cell count in the hippocampus area of the rats.

FIG. 7A shows Iba1 staining in the cortex of rats induced with TBI by controlled cortical impact (CCI) and administered with HMCs or vehicle intracerebrally (IC) or intravenously (IV). FIG. 7B shows a bar graph of the Iba1 cell count in the cortex of the rats.

FIG. 7C shows Iba1 staining in the striatum rats induced with TBI by controlled cortical impact (CCI) and administered with HMCs or vehicle intracerebrally (IC) or intravenously (IV). FIG. 7D shows a bar graph of the Iba1 cell count in the striatum of the rats.

FIG. 8A shows OX6 staining of the cortex of rats induced with TBI by controlled cortical impact (CCI) and administered with HMCs or vehicle intracerebrally (IC) or intravenously (IV). FIG. 8B shows a bar graph of the OX6 cell count in the cortex of the rats.

FIG. 8C shows OX6 staining of the striatum of rats induced with TBI by controlled cortical impact (CCI) and administered with HMCs or vehicle intracerebrally (IC) or intravenously (IV). FIG. 8D shows a bar graph of the OX6 cell count in the striatum of the rats.

FIG. 9A shows 1L6 staining in the spleens of rats induced with TBI by controlled cortical impact (CCI) and administered with HMCs or vehicle intracerebrally (IC) or intravenously (IV). FIG. 9B shows a bar graph of the 1L6 staining intensity in the spleens of the rats.

FIG. 10A shows TNF-alpha staining in the spleens of rats induced with TBI by controlled cortical impact (CCI) and administered with HMCs or vehicle intracerebrally (IC) or intravenously (IV). FIG. 10B shows a bar graph of the TNF-alpha staining intensity in the spleens of the rats.

FIG. 11A shows HuNu staining in the cortex of rats induced with TBI by controlled cortical impact (CCI) and administered with HMCs or vehicle intracerebrally (IC) or intravenously (IV). FIG. 11B shows a bar graph of the HuNu cell count in the cortex of the rats. FIG. 11C shows HuNu staining in the striatum of rats induced with TBI by controlled cortical impact (CCI) and administered with HMCs or vehicle intracerebrally (IC) or intravenously (IV). FIG. 11D shows a bar graph of the HuNu cell count in the striatum of the rats. FIG. 11E shows HuNu staining in the hippocampus of rats induced with TBI by controlled cortical impact (CCI) and administered with HMCs or vehicle intracerebrally (IC) or intravenously (IV). FIG. 11F shows a bar graph of the HuNu cell count in the hippocampus of the rats.

FIG. 12A shows migration of unstimulated hESC-MSCs (“HMC”), BM-MSCs, and UCB-MSCs into a gap of about 500 μm wide at 0 hrs and 6 hrs. FIG. 12B shows a bar graph of the number of unstimulated and stimulated cells that had migrated into the gap.

FIG. 13 shows images of neurite outgrowth staining at days 1 and 7 post-scratch and co-culture of hESC-MSCs (“HMC”) with a mixed neuronal culture.

FIG. 14A shows TUNEL ranking of each rat tested in the in vivo neonatal hypoxia-ischemia model of cerebral palsy. FIG. 14B shows a bar graph of the average TUNEL ranking of each group of rats tested. TUNEL ranking was as follows: 1=no structural damage and No TUNEL; 2=structural damage and Low TUNEL; 3=structural damage and Medium TUNEL; 4=structural damage and High TUNEL; 5=extreme damage/tissue gone. A comparison of the rats in the Sham vs HI groups showed a t-test of 0.006284 and Mann-Whitney of 0.0256; Sham vs Lot B groups showed a t-test of 0.148904 and Mann-Whitney of 0.2; and HI vs Lot B groups showed a t-test of 0.101453 and Mann-Whitney of 0.1841.

FIG. 15 shows H&E staining of the brains of rats tested in the in vivo neonatal hypoxia-ischemia model of cerebral palsy.

FIG. 16A shows images of Iba-1 staining in peri-infarct tissue of rats tested in the in vivo neonatal hypoxia-ischemia model of cerebral palsy. FIG. 16B shows the mean signal intensity of Iba-1 staining in each rat tested in the in vivo neonatal hypoxia-ischemia model of cerebral palsy. FIG. 16C shows the average mean signal intensity of Iba-1 staining in each group of rats tested. A comparison of the rats in the Sham vs HI groups showed a t-test of 0.039335 and Mann-Whitney of 0.065; Sham vs Lot B groups showed a t-test of 0.129562 and Mann-Whitney of 0.1949; and HI vs Lot B groups showed a t-test of 0.353204 and Mann-Whitney of 0.4418.

FIG. 17A shows images of GFAP staining in peri-infarct tissue of rats tested in the in vivo neonatal hypoxia-ischemia model of cerebral palsy. FIG. 17B shows the mean signal intensity of GFAP staining in each rat tested in the in vivo neonatal hypoxia-ischemia model of cerebral palsy. FIG. 17C shows the average mean signal intensity of GFAP staining in each group of rats tested. A comparison of the rats in the Sham vs HI groups showed a t-test of 0.011749 and Mann-Whitney of 0.0047; Sham vs Lot B groups showed a t-test of 0.070012 and Mann-Whitney of 0.0207; and HI vs Lot B groups showed a t-test of 0.57941 and Mann-Whitney of 0.7984.

FIG. 18A shows images of MBP staining in the corpus callosum in rats tested in the in vivo neonatal hypoxia-ischemia model of cerebral palsy. FIG. 18B shows the mean signal intensity of MBP staining in each rat tested in the in vivo neonatal hypoxia-ischemia model of cerebral palsy. FIG. 18C shows the average mean signal intensity of MBP staining in each group of rats tested. A comparison of the rats in the Sham vs HI groups showed a t-test of 0.012963 and Mann-Whitney of 0.007; Sham vs Lot B groups showed a t-test of 0.189251 and Mann-Whitney of 0.3282; and HI vs Lot B groups showed a t-test of 0.172857 and Mann-Whitney of 0.2345.

FIG. 19A shows images of Olig2 staining in the hippocampus of the ipsilesional hemisphere of rats tested in the in vivo neonatal hypoxia-ischemia model of cerebral palsy.

FIG. 19B shows the mean signal intensity of Olig2 staining in the SVZ, cortex, hippocampus, and region mean of each rat tested in the in vivo neonatal hypoxia-ischemia model of cerebral palsy. FIG. 19C shows the average mean signal intensity of Olig2 staining in the SVZ, cortex, hippocampus, and region mean of each group of rats tested. A comparison of the rats in Lot B vs HI for Olig2 staining in the SVZ showed a t-test of 0.3962; in the cortex a t-test of 0.4399; in the hippocampus a t-test of 0.5435; and the region mean showed a t-test of 0.3597.

FIG. 20 depicts the results of the body swing test in rats having middle cerebral artery occlusion (MCAO) stroke and receiving HMCs via three routes of administration: intravenous (IV), intracerebral (IC) and intrathecal (IT) administration. Two-way ANOVA with Tukey's MCT was used for statistical analysis, *p<0.05, **p<0.01, ***p<0.001, and ****p<0.0001.

FIG. 21 depicts the results of the forelimb placement, the hindlimb placement, and the body swing test in rats having middle cerebral artery occlusion (MCAO) stroke and receiving HMCs and HMC-EVs via intravenous, intracerebral and intracisternal administration. Two-way ANOVA Tukey's MCT was used for statistical analysis, *p<0.05, **p<0.01, ***p<0.001, and ****p<0.0001.

FIG. 22 depicts the results of the forelimb placement, the hindlimb placement, and the body swing test in rats having middle cerebral artery occlusion (MCAO) stroke and receiving HMC-EVs via intracisternal administration. Two-way ANOVA with Tukey's MCT was used for statistical analysis, *p<0.05, **p<0.01, ***p<0.001, and ****p<0.0001.

FIG. 23 depicts the results of the forelimb placement, the hindlimb placement, and the body swing test in rats having middle cerebral artery occlusion (MCAO) stroke and receiving HMC-EVs via intrathecal administration. Two-way ANOVA with Turkey's MCT was used for statistical analysis, *p<0.05, **p<0.01, ***p<0.001, and ****p<0.0001.

FIG. 24A shows images of MBP staining in the cortex and striatum in rats having MCAO stroke and receiving HMCs (obtained from C-GS1 and N-line cells) via IV administration. FIG. 24B shows the average signal intensity of MBP staining in the cortex of rats tested in the vivo MCAO stroke model. FIG. 24C shows the average signal intensity of MBP staining in the striatum of rats tested in the vivo MCAO stroke model. For sham vs Vehicle groups: Welch's test was used for statistical analysis, ***p<0.001. For vehicle vs treatment groups: one-way ANOVA with Dunnet's multiple comparisons test was used for statistical analysis, *p<0.05, **p<0.01, and ***P<0.001.

FIG. 25A shows images of Iba1 staining in the cortex and striatum in rats having MCAO stroke and receiving HMCs (obtained from C-GS1 and N-line cells) via IV administration. FIG. 25B shows the average signal intensity of Iba1 staining in the cortex of rats tested in the vivo MCAO stroke model. FIG. 25C shows the average signal intensity of Iba1 staining in the striatum of rats tested in the vivo MCAO stroke model. For sham vs Vehicle groups: Welch's test was used for statistical analysis, ***p<0.001. For vehicle vs treatment groups: one-way ANOVA with Dunnet's multiple comparisons test was used for statistical analysis, *p<0.05, **p<0.01, and ***P<0.001.

FIG. 26A shows images of GFAP staining in the cortex and striatum in rats having MCAO stroke and receiving HMCs (obtained from C-GS1 and N-line cells) via IV administration. FIG. 26B shows the average signal intensity of GFAP staining in the cortex of rats tested in the vivo MCAO stroke model. FIG. 26C shows the average signal intensity of GFAP staining in the striatum of rats tested in the vivo MCAO stroke model. For sham vs Vehicle groups: Welch's test was used for statistical analysis, ***p<0.001. For vehicle vs treatment groups: one-way ANOVA with Dunnet's multiple comparisons test was used for statistical analysis, *p<0.05, **p<0.01, and ***P<0.001.

FIG. 27A shows images of MBP staining in rats having MCAO stroke and receiving HMC-EVs (obtained from N-line cells, treated with IFNgamma for 96 hours at 50 ng/mL) via intracisternal administration. FIG. 27B shows the average signal intensity of MBP staining in rats tested in the vivo MCAO stroke model. cc: corpur callosum; ec: external capsule; cg: cingulate gyrus. For vehicle vs treatment groups: Bonferroni comparisons was used for statistical analysis, **p<0.01.

FIG. 28A shows images of Iba1 staining in rats having MCAO stroke and receiving HMC-EVs (obtained from N-line cells, treated with gamma interferon for 96 hours at 50 ng/mL) via intracisternal administration. FIG. 28B shows the average signal intensity of Iba1 staining in rats tested in the vivo MCAO stroke model. cc: corpur callosum; ec: external capsule; cg: cingulate gyrus. For vehicle vs treatment groups: Bonferroni comparisons was used for statistical analysis, **p<0.01.

FIG. 29A shows images of GFAP staining in rats having MCAO stroke and receiving HMC-EVs (obtained from N-line cells, treated with gamma interferon for 96 hours at 50 ng/mL) via intracisternal administration. FIG. 29B shows the average signal intensity of GFAP staining in rats tested in the vivo MCAO stroke model. cc: corpur callosum; ec: external capsule; cg: cingulate gyrus. For vehicle vs treatment groups: Bonferroni comparisons was used for statistical analysis, **p<0.01.

FIG. 30A shows images of Olig2 staining in rats having MCAO stroke and receiving HMC-EVs (obtained from N-line cells, treated with gamma interferon for 96 hours at 50 ng/mL) via intracisternal administration. FIG. 30B shows the average signal intensity of Olig2 staining in rats tested in the vivo MCAO stroke model. cc: corpur callosum; ec: external capsule; cg: cingulate gyrus. For vehicle vs treatment groups: Bonferroni comparisons was used for statistical analysis, **p<0.01.

FIG. 31A shows images of NG2 staining in rats having MCAO stroke and receiving HMC-EVs (obtained from N-line cells, treated with gamma interferon for 96 hours at 50 ng/mL) via intracisternal administration. FIG. 30B shows the average signal intensity of NG2 staining in rats tested in the vivo MCAO stroke model. cc: corpur callosum; ec: external capsule; cg: cingulate gyrus. For vehicle vs treatment groups: Bonferroni comparisons was used for statistical analysis, **p<0.01.

FIG. 32 is a schematic of the study design for the in vitro oxygen glucose deprivation (OGD) assay for modeling stroke.

FIG. 33A shows TUNEL staining and imaging of primary rat neurons treated with or without HMCs following 0 hr, 1 hr, 2 hr and 3 hr oxygen glucose deprivation (OGD) injury.

FIG. 33B shows the average TUNEL quantification of primary rat neurons treated with or without MSCs following 0 hr, 1 hr, 2 hr and 3 hr OGD injury.

FIGS. 34A-F depict the pathway enrichment analysis of the differential expression between neurons subjected to 3 hours of oxygen glucose deprivation injury and grown on HMC-enriched and control media. FIGS. 34A-B depict the pathways enriched by the differential expression. FIGS. 34C-F depict the differential expression between OGD neurons grown on HMC-enriched and control media for Gene Oncology terms. FIG. 34C shows the upregulation of pathways involved in cell viability, neuroprotection, and synaptic transmission in OGD neurons grown on HMC-enriched culture. FIG. 34D shows upregulation of genes involved in neuroprotection in OGD neurons grown on HMC-enriched culture. FIG. 34E shows the downregulation of pathways involved in apoptosis in OGD neurons grown on HMC-enriched culture. FIG. 34F shows downregulation of genes involved in apoptosis or general response to cell death in OGD neurons grown on HMC-enriched culture.

FIG. 35A depicts the in vitro OGD assay RNAseq analysis of primary rat neurons treated with or without HMCs following 0 hr, 1 hr, 2 hr and 3 hr oxygen glucose deprivation (OGD) injury. FIG. 35B depicts the qPCR analysis of primary rat neurons treated with or without HMCs following 0 hr, 1 hr, 2 hr and 3 hr oxygen glucose deprivation (OGD) injury. Two-way ANOVA with Sidak multiple comparison test was used for statistical analysis: *p<0.05, **p<0.01, and ****p<0.0001.

FIG. 36A shows attenuation of cell death by HMC-EVs. Percentage of cell death was determined as the number of PI+ cells out of the total Hoechst+ cells. Two-way ANOVA was used for statistical significance analysis. ****p<0.0001. FIG. 36B shows dose-dependent attenuation of cell death by HMC-EV treatment. Percentage of cell death was determined as the number of PI+ cells out of the total Hoechst+ cells. One-way

FIG. 37 shows maintenance of the mitochondrial membrane potential in HMC-EV treated cells undergoing nuclear swelling. HMC-EV treatment sustained cells in the nuclear swelling stage after glutamate-induced injury.

FIG. 38 shows the principal component analysis of transcriptomes of HMCs (obtained from N-line cells), and adipose tissue-derived MSCs shows that HMCs are distinct from adipose tissue-derived MSCs in both basal and inteferon-gamma stimulated state. AMSC-B-1,2,3: adipose tissue-derived MSCs collected from 3 different adult donors, 2 technical replicate samples for each biological replicates. AMSC-S-1,2,3: adipose tissue-derived MSCs, but stimulated with gamma interferon. NHMC-B: 3 technical replicates of MSCs derived from N-line cells, basal state. NHMC-S: MSCs derived from N-line cells, but stimulated with gamma interferon.

FIG. 39 depicts the weights of different genes contributing to the second principal component which determines the variance between HMCs (obtained from N-line cells) and adipose tissue-derived MSCs.

FIG. 40 depicts the hierarchical clustering map demonstrating that HMCs (obtained from N-line cells) are distinct from adipose tissue-derived MSCs in both basal and gamma interferon-stimulated states. AB1, AB2, AB3—adipose tissue-derived MSCs collected from 3 different adult donors, 2 technical replicates per donor; basal cell state. AS1, AS2, AS3-adipose tissue-derived MSCs, stimulated with gamma interferon. NB—MSCs derived from N-line cells, basal states, 3 technical replicates. NS—MSCs derived from N-line cells, stimulated with gamma interferon.

FIG. 41 depicts the basal HMC-specific cluster of genes.

FIG. 42 depicts the basal adipose tissue-derived MSC-specific cluster of genes.

FIG. 43 depicts the pathway enrichment of differential expression pattern between HMCs (obtained from N-line cells) and adipose tissue-derived MSCs showing noticeable HMC-specific up-regulation of several pathways (denoted by arrows) involved in the development of neuronal lineage including axon guidance, CREB signaling in neurons, and synaptogenesis signaling.

FIG. 44 depicts the top 15 most strongly differentially expressed genes contributing to activation of neuronal CREB signaling in HMCs (obtained from N-line cells).

FIG. 45 depicts the top 15 most strongly upregulated genes contributing to the enrichment of axon guidance pathway in HMCs (obtained from N-line cells).

FIG. 46 depicts the top 15 most strongly expressed genes contributing to activation of synaptogenesis signaling pathway in HMCs (obtained from N-line cells).

FIG. 47 depicts the top 15 most up-regulated genes contributing to activation of neuroinflammation signaling pathway in HMCs (obtained from N-line cells).

FIG. 48 shows the principal component analysis of transcriptomes of HMCs obtained from N-line cells, HMCs obtained from GMP1 cells, and adipose tissue-derived MSCs. AMSC-B-1,2,3—adipose tissue-derived MSCs collected from 3 different adult donors, basal state, 2 technical replicate samples for each biological replicate. AMSC-S-1,2,3—adipose tissue-derived MSCs collected from 3 different adult donors, but stimulated with gamma interferon. NHMC-B—HMCs derived from N-line cells, basal state. NHMC-S—HMCs derived from N-line cells, but stimulated with gamma interferon. GMP-B—HMC derived from GMP1 cell line, basal state. GMP-S—HMC derived from GMP1 cell line, but stimulated with gamma interferon.

FIG. 49 depicts the hierarchical clustering map demonstrating that HMCs (obtained from N-line cells) and HMCs (obtained from GMP1 cells) are distinct from adipose tissue-derived MSCs in both basal and gamma interferon-stimulated cell states. AB1, AB2, AB3—adipose tissue-derived MSCs collected from 3 different adult donors, 2 technical replicates per donor; basal cell state. AS1, AS2, AS3—adipose tissue-derived MSCs collected from 3 different adult donors, stimulated with gamma interferon. NB—HMCs derived from N-line cells, basal state, 3 technical replicates. NS—HMCs derived from N-line cells, stimulated with gamma interferon. GB—HMC derived from GMP1 cell line, basal state, 3 technical replicates. GS—HMC derived from GMP1 cell line, stimulated with gamma interferon.

FIG. 50 depicts the HMC-specific cluster of genes.

FIG. 51 depicts the basal adipose tissue-derived MSC-specific cluster of genes.

FIG. 52 depicts the stimulated adipose tissue-derived MSC-specific cluster of genes.

FIG. 53A depicts the pathway enrichment of differential expression pattern between HMCs (obtained from GMP1 cells) and adipose tissue-derived MSCs showing noticeable HMC-specific up-regulation of several pathways involved in the development of neuronal lineage including axon guidance, CREB signaling in neurons, and synaptogenesis signaling.

FIG. 53B depicts the top canonical pathways that are differentially regulated in HMCs. FIG. 53C depicts exemplary regulators being activated and inhibited in HMCs.

FIG. 54A depicts the pathway enrichment of differential expression pattern between HMCs (obtained from N-line cells) and adipose tissue-derived MSCs showing noticeable HMC-specific up-regulation of several pathways involved in the development of neuronal lineage including axon guidance, CREB signaling in neurons, and synaptogenesis signaling.

FIG. 54B depicts the top canonical pathways that are differentially regulated in HMCs. FIG. 54C depicts exemplary regulators being activated and inhibited in HMCs.

FIG. 55 shows the principal component analysis of transcriptomes of HMCs (obtained from N-line cells) and bone marrow-derived MSCs shows that HMCs are distinct from bone marrow-derived MSCs in both basal and inteferon-gamma stimulated states. BM-B—bone marrow-derived MSCs collected from 3 different adult donors, basal states, 2 technical replicate samples for each biological replicate. BM-S—bone marrow-derived MSCs, but stimulated with gamma interferon. N-B—3 technical replicates of HMCs derived from N-line cells, basal state. N-S—HMCs derived from N-line cells, but stimulated with gamma interferon.

FIG. 56 depicts the weights of different genes contributing to the second principal component which determines the variance between HMCs and bone marrow-derived MSCs.

FIG. 57 depicts the hierarchical clustering map demonstrating that HMCs (obtained from N-line cells) are distinct from bone marrow-derived MSCs in both basal and gamma interferon-stimulated cell states. BMB1, BMB2, BMB3—bond marrow-derived MSCs collected from 3 different adult donors, 2 technical replicates per donor; basal cell state. BMS1, BMS2, BMS3—bond marrow-derived MSCs, stimulated with gamma interferon. NB—HMCs derived from N-line cells, basal states, 3 technical replicates. NS—HMCs derived from N-line cells, stimulated with gamma interferon.

FIG. 58 depicts the basal HMC-specific cluster of genes.

FIG. 59 depicts the basal bone marrow-derived MSC-specific cluster of genes.

FIG. 60 depicts the pathway enrichment of differential expression pattern between HMCs (obtained from N-line cells) and bone marrow-derived MSCs showing noticeable HMC-specific up-regulation of several pathways (denoted by arrows) involved in the development of neuronal lineage such as CREB signaling in neurons.

FIG. 61 depicts the top 15 most strongly differentially expressed genes contributing to activation of neuronal CREB signaling in HMCs (obtained from N-line cells).

FIG. 62 depicts the top 15 most strongly upregulated genes contributing to activation of synaptogenesis signaling in HMCs (obtained from N-line cells).

FIG. 63A depicts the pathway enrichment of differential expression pattern between HMC-EVs and EVs secreted from bone marrow-derived MSCs (BM-MSC-EVs). Pathways that are upregulated in HMC-EVs have a positive z-score and are represented by orange bars. Pathways that are downregulated in HMC-EVs have a negative z-score and are represented by blue bars. White/gray bars represent pathways that are enriched in HMC-EVs, i.e., proteins contributing to these pathways are enriched. FIG. 63B depicts the disease or functional annotation of proteins that have higher expression levels in HMC-EVs when compared to BM-MSC-EVs. FIG. 63C depicts the disease or functional annotation of proteins that have lower expression levels in HMC-EVs when compared to BM-MSC-EVs. An activation z-score above 2 or below −2 is considered as the threshold value.

FIG. 64A depicts the pathway enrichment of differential expression pattern between HMC-EVs and EVs secreted from adipose tissue-derived MSCs (AD-MSC-EVs). Pathways that are upregulated in HMC-EVs have a positive z-score and are represented by orange bars. Pathways that are downregulated in HMC-EVs have a negative z-score and are represented by blue bars. White/gray bars represent pathways that are enriched in HMC-EVs, i.e., proteins contributing to these pathways are enriched. FIG. 64B depicts the disease or function annotational of proteins that have higher expression levels in HMC-EVs when compared to AD-MSC-EVs. FIG. 64C depicts the disease or function annotational of proteins that have lower expression levels in HMC-EVs when compared to AD-MSC-EVs. An activation z-score above 2 or below −2 is considered as the threshold value.

FIG. 65A depicts the pathway enrichment of differential expression pattern between HMC-EVs and EVs secreted from umbilical cord blood-derived MSCs (UCB-MSC-EVs). Pathways that are upregulated in HMC-EVs have a positive z-score and are represented by orange bars. Pathways that are downregulated in HMC-EVs have a negative z-score and are represented by blue bars. White/gray bars represent pathways that are enriched in HMC-EVs, i.e., proteins contributing to these pathways are enriched. FIG. 65B depicts the disease or function annotational of proteins that have higher expression levels in HMC-EVs when compared to UCB-MSC-EVs. FIG. 65C depicts the disease or function annotational of proteins that have lower expression levels in HMC-EVs when compared to UCB-MSC-EVs. An activation z-score above 2 or below −2 is considered as the threshold value.

### DETAILED DESCRIPTION

### Definitions

“Pluripotent cells”, “pluripotent stem cells,” and “PSCs” as used herein, refer broadly to a cell capable of prolonged or virtually indefinite proliferation in vitro while retaining their undifferentiated state, exhibiting a stable (preferably normal) karyotype, and having the capacity to differentiate into all three germ layers (i.e., ectoderm, mesoderm and endoderm) under the appropriate conditions. Typically pluripotent cells (a) are capable of inducing teratomas when transplanted in immunodeficient (SCID) mice; (b) are capable of differentiating to cell types of all three germ layers (e.g., ectodermal, mesodermal, and endodermal cell types); and (c) express at least one hES cell marker (such as Oct-4, alkaline phosphatase, SSEA 3 surface antigen, SSEA 4 surface antigen, NANOG, TRA 1 60, TRA 1 81, SOX2, REX1). Exemplary pluripotent cells may express Oct-4, alkaline phosphatase, SSEA 3 surface antigen, SSEA 4 surface antigen, TRA 1 60, and/or TRA 1 81. Additional exemplary pluripotent cells include but are not limited to embryonic stem cells, induced pluripotent cells (iPS) cells, embryo-derived cells, pluripotent cells produced from embryonic germ (EG) cells (e.g., by culturing in the presence of FGF-2, LIF and SCF), parthenogenetic ES cells, ES cells produced from cultured inner cell mass cells (ICM), ES cells produced from a blastomere, and ES cells produced by nuclear transfer (e.g., a somatic cell nucleus transferred into a recipient oocyte). Exemplary pluripotent cells may be produced without destruction of an embryo. For example, induced pluripotent cells may be produced from cells obtained without embryo destruction. As a further example, pluripotent cells may be produced from a biopsied blastomere (which can be accomplished without harm to the remaining embryo); optionally, the remaining embryo may be cryopreserved, cultured, and/or implanted into a suitable host. Pluripotent cells (from whatever source) may be genetically modified or otherwise modified to increase longevity, potency, homing, or to deliver a desired factor in cells that are differentiated from such pluripotent cells (for example, MSCs, and hemangioblasts). As non-limiting examples thereof, the pluripotent cells may be genetically modified to express Sirt1 (thereby increasing longevity), express one or more telomerase subunit genes optionally under the control of an inducible or repressible promoter, incorporate a fluorescent label, incorporate iron oxide particles or other such reagent (which could be used for cell tracking via in vivo imaging, MRI, etc., see Thu et al., Nat Med. 2012 Feb. 26; 18(3):463-7), express bFGF which may improve longevity (see Go et al., J. Biochem. 142, 741-748 (2007)), express CXCR4 for homing (see Shi et al., Haematologica. 2007 Jul; 92(7):897-904), express recombinant TRAIL to induce caspase-mediated apoptosis in cancer cells like Gliomas (see Sasportas et al., Proc Natl Acad Sci USA. 2009 Mar. 24; 106(12):4822-7), etc.

“Embryo” or “embryonic,” as used herein refers broadly to a developing cell mass that has not implanted into the uterine membrane of a maternal host. An “embryonic cell” is a cell isolated from or contained in an embryo. This also includes blastomeres, which may be obtained as early as the two-cell stage, and aggregated blastomeres.

“Embryonic stem cells” (ES cells or ESC) encompasses pluripotent cells produced from embryonic cells (such as from cultured inner cell mass cells or cultured blastomeres). Frequently such cells are or have been serially passaged as cell lines. Embryonic stem cells may be used as a pluripotent stem cell in the processes of producing hemangioblasts as described herein. For example, ES cells may be produced by methods known in the art including derivation from an embryo produced by any method (including by sexual or asexual means) such as fertilization of an egg cell with sperm or sperm DNA, nuclear transfer (including somatic cell nuclear transfer), or parthenogenesis. As a further example, embryonic stem cells also include cells produced by somatic cell nuclear transfer, even when non-embryonic cells are used in the process. For example, ES cells may be derived from the ICM of blastocyst stage embryos, as well as embryonic stem cells derived from one or more blastomeres. Such embryonic stem cells can be generated from embryonic material produced by fertilization or by asexual means, including somatic cell nuclear transfer (SCNT), parthenogenesis, and androgenesis. As further discussed above (see “pluripotent cells), ES cells may be genetically modified or otherwise modified to increase longevity, potency, homing, or to deliver a desired factor in cells that are differentiated from such pluripotent cells (for example, MSCs, and hemangioblasts).

ES cells may be generated with homozygosity or hemizygosity in one or more HLA genes, e.g., through genetic manipulation, screening for spontaneous loss of heterozygosity, etc. day ES cells may be genetically modified or otherwise modified to increase longevity, potency, homing, or to deliver a desired factor in cells that are differentiated from such pluripotent cells (for example, MSCs and hemangioblasts). Embryonic stem cells, regardless of their source or the particular method used to produce them, typically possess one or more of the following attributes: (i) the ability to differentiate into cells of all three germ layers, (ii) expression of at least Oct-4 and alkaline phosphatase, and (iii) the ability to produce teratomas when transplanted into immunocompromised animals. Embryonic stem cells that may be used in embodiments of the presently disclosed subject matter include, but are not limited to, human ES cells (“hESC” or “hES cells”) such as CT2, MA01, MA09, ACT-4, No. 3, H1, H7, H9, H14 and ACT30 embryonic stem cells. Additional exemplary cell lines include NED1, NED2, NED3, NED4, NED5, and NED7. See also NIH Human Embryonic Stem Cell Registry. An exemplary human embryonic stem cell line that may be used is MA09 cells. The isolation and preparation of MA09 cells was previously described in Klimanskaya, et al. (2006) “Human Embryonic Stem Cell lines Derived from Single Blastomeres.” Nature 444: 481-485. The human ES cells used in accordance with exemplary embodiments of the presently disclosed subject matter may be derived and maintained in accordance with GMP standards.

Exemplary hES cell markers include, but are not limited to: alkaline phosphatase, Oct-4, Nanog, Stage-specific embryonic antigen-3 (SSEA-3), Stage-specific embryonic antigen-4 (SSEA-4), TRA-1-60, TRA-1-81, TRA-2-49/6E, Sox2, growth and differentiation factor 3 (GDF3), reduced expression 1 (REX1), fibroblast growth factor 4 (FGF4), embryonic cell-specific gene 1 (ESG1), developmental pluripotency-associated 2 (DPPA2), DPPA4, telomerase reverse transcriptase (hTERT), SALL4, E-CADHERIN, Cluster designation 30 (CD30), Cripto (TDGF-1), GCTM-2, Genesis, Germ cell nuclear factor, and Stem cell factor (SCF or c-Kit ligand). Additionally, embryonic stem cells may express Oct-4, alkaline phosphatase, SSEA 3 surface antigen, SSEA 4 surface antigen, TRA 1 60, and/or TRA 1 81.

The ESCs may be initially co-cultivated in any culture media known in the art that maintains the pluripotency of the ESCs, with or without feeder cells, such as murine embryonic feeder cells (MEF) cells or human feeder cells, such as human dermal fibroblasts (HDF). The MEF cells or human feeder cells may be mitotically inactivated, for example, by exposure to mitomycin C, gamma irradiation, or by any other known methods, prior to seeding ESCs in co-culture, and thus the MEFs do not propagate in culture. Additionally, ESC cell cultures may be examined microscopically and colonies containing non ESC cell morphology may be picked and discarded, e.g., using a stem cell cutting tool, by laser ablation, or other means. Typically, after the point of harvest of the ESCs for seeding for embryoid body formation no additional MEF cells or human feeder cells are used.

Alternatively, hES cells may be cultured under feeder-free conditions on a solid surface such as an extracellular matrix e.g. by any method known in the art, e.g., Klimanskaya et al., Lancet 365:1636-1641 (2005). Accordingly, the hES cells used in the methods described herein may be cultured on feeder-free cultures.

“Embryo-derived cells” (EDC), as used herein, refers broadly to pluripotent morula-derived cells, blastocyst-derived cells including those of the inner cell mass, embryonic shield, or epiblast, or other pluripotent stem cells of the early embryo, including primitive endoderm, ectoderm, and mesoderm and their derivatives. “EDC” also including blastomeres and cell masses from aggregated single blastomeres or embryos from varying stages of development, but excludes human embryonic stem cells that have been passaged as cell lines.

“Potency”, as used herein, refers broadly to the concentration, e.g., number of cells (such as hemangioblast-derived MSCs) that produces a defined effect. Potency may be defined in terms of effective concentration (EC50), which does not involve measurements of maximal effect but, instead, the effect at various locations along the concentration axis of dose response curves. Potency may also be determined from either graded (EC50) or quantal dose-response curves (ED50, TD50 and LD50); however, potency is preferably measured by EC50. The term “EC50” refers to the concentration of a drug, antibody or toxicant which induces a response halfway between the baseline and maximum effect after some specified exposure time. The EC50 of a graded dose response curve therefore represents the concentration of a compound where 50% of its maximal effect is observed. The EC50 of a quantal dose response curve represents the concentration of a compound where 50% of the population exhibit a response, after a specified exposure duration. The EC50 may be determined using animal studies in which a defined animal model demonstrates a measurable, physiological change in response to application of the drug; cell-based assays that use a specified cell system, which on addition of the drug, demonstrate a measurable biological response; and/or enzymatic reactions where the biological activity of the drug can be measured by the accumulation of product following the chemical reaction facilitated by the drug. Preferably, an immune regulatory assay is used to determine EC50. Non-limiting examples of such immune regulatory assays include intracellular cytokine, cytotoxicity, regulatory capacity, cell signaling capacity, proliferative capacity, apoptotic evaluations, and other assays.

“Mesenchymal stem cells” (MSCs) as used herein refers to multipotent stem cells with self-renewal capacity and the ability to differentiate into osteoblasts, chondrocytes, and adipocytes, among other mesenchymal cell lineages. Unless otherwise specifically noted, MSCs of the presently disclosed subject matter are MSCs generated from in vitro differentiation of pluripotent stem cells, and which may be referred to herein as HMCs. In an embodiment, the HMCs may be generated by in vitro differentiation of pluripotent stem cells followed by differentiation to hemangioblasts, which are then differentiated into HMCs. HMCs may be identified by the expression of one or more markers as further described herein. HMCs may also have any of the characteristics described in WO 2013/082543, U.S. Pat. Nos. 8,962,321, and 8,961,956, the entire contents of which are hereby incorporated herein by reference.

HMCs may be genetically modified or otherwise modified to increase longevity, potency, homing, or to deliver a desired factor in the HMCs or cells that are differentiated from such HMCs. As non-limiting examples thereof, the HMCs may be genetically modified to express Sirt1 (thereby increasing longevity), express one or more telomerase subunit genes optionally under the control of an inducible or repressible promoter, incorporate a fluorescent label, incorporate iron oxide particles or other such reagent (which could be used for cell tracking via in vivo imaging, MRI, (see Thu et al., Nat Med. 2012 Feb. 26; 18(3):463-7), express bFGF which may improve longevity (see Go et al., J. Biochem. 142, 741-748 (2007)), express CXCR4 for homing (see Shi et al., Haematologica. 2007 July; 92(7):897-904), express recombinant TRAIL to induce caspase-mediated apoptosis in cancer cells like Gliomas (see Sasportas et al., Proc Natl Acad Sci USA. 2009 Mar. 24; 106(12):4822-7).

As used herein, the term “extracellular vesicle” or “EV” refers to lipid bound vesicles secreted by cells into the extracellular space. The three main subtypes of EVs are microvesicles (MVs), exosomes, and apoptotic bodies, which are differentiated based upon their biogenesis, release pathways, size, content, and function (Zaborowski M. P., et al. Bioscience. 2015; 65:783-797). Generally extracellular vesicles range in diameter from 20 nm to 5000 nm, and can comprise various macromolecular payload either within the internal space (i.e., lumen), displayed on the external surface of the extracellular vesicle, and/or spanning the membrane. Said payload can comprise nucleic acids, e.g., microRNAs (miRNA), long non-coding RNAs (lncRNA), mRNAs, DNA fragments; proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived/secreted from a living or dead organism, explanted tissues or organs, prokaryotic or eukaryotic cells, and/or cultured cells.

“Optic neuropathy”, as used herein, includes any disease, disorder or condition that involves damage to the optic nerve. Optic neuropathy includes hereditary (e.g., autosomal dominant optic atrophy (Kjer's disease) and maternally inherited Leber's hereditary optic neuropathy) and non-hereditary optic neuropathy (e.g., ischemic optic neuropathy). In one embodiment, optic neuropathy is glaucoma/glaucomatic optic neuropathy.

“Therapy,” “therapeutic,” “treating,” “treat” or “treatment”, as used herein, refers broadly to treating a disease, arresting or reducing the development of the disease or its clinical symptoms, and/or relieving the disease, causing regression of the disease or its clinical symptoms. “Therapy”, “therapeutic,” “treating,” “treat” or “treatment” encompasses prophylaxis, prevention, treatment, cure, remedy, reduction, alleviation, and/or providing relief from a disease, signs, and/or symptoms of a disease. “Therapy”, “therapeutic,” “treating,” “treat” or “treatment” encompasses an alleviation of signs and/or symptoms in patients with ongoing disease signs and/or symptoms. “Therapy”, “therapeutic,” “treating,” “treat” or “treatment” also encompasses “prophylaxis” and “prevention”. Prophylaxis includes preventing disease occurring subsequent to treatment of a disease in a patient or reducing the incidence or severity of the disease in a patient. The term “reduced”, for purpose of therapy, “therapeutic,” “treating,” “treat” or “treatment” refers broadly to the clinical significant reduction in signs and/or symptoms. “Therapy”, “therapeutic,” “treating,” “treat” or “treatment” includes treating relapses or recurrent signs and/or symptoms. “Therapy”, “therapeutic,” “treating,” “treat” or “treatment” encompasses but is not limited to precluding the appearance of signs and/or symptoms anytime as well as reducing existing signs and/or symptoms and eliminating existing signs and/or symptoms. “Therapy”, “therapeutic,” “treating,” “treat” or “treatment” includes treating chronic disease (“maintenance”) and acute disease. For example, treatment includes treating or preventing relapses or the recurrence of signs and/or symptoms.

As used herein, the term “effective amount,” is intended to include the amount of HMCs and/or HMC-EVs that, when administered to a subject having a brain injury, is sufficient to effect treatment of the disease (e.g., by diminishing, ameliorating, or maintaining the existing disease or one or more symptoms of disease). Ameliorating the disease includes slowing the course of the disease or reducing the severity of later-developing disease. The “effective amount” may vary depending on the nature of the HMC and/or HMC-EVs, how the HMC and/or HMC-EVs are administered, the disease and its severity and the history, age, weight, family history, genetic makeup, the types of preceding or concomitant treatments, if any, and other individual characteristics of the subject to be treated.

An “effective amount” also includes an amount of HMC and/or HMC-EVs that produces some desired effect at a reasonable benefit/risk ratio applicable to any treatment. The HMC and/or HMC-EVs employed in the methods of the presently disclosed subject matter may be administered in a sufficient amount to produce a reasonable benefit/risk ratio applicable to such treatment.

“Normalizing a pathology”, as used herein, refers to reverting the abnormal structure and/or function resulting from a disease to a more normal state. Normalization suggests that by correcting the abnormalities in structure and/or function of a tissue, organ, cell type, etc. resulting from a disease, the progression of the pathology can be controlled and improved. For example, following treatment with the HMCs of the presently disclosed subject matter the abnormalities of the brain as a result of brain injury, e.g., traumatic brain injury, may be improved, corrected, and/or reversed.

“Induced pluripotent stem cells” or “iPSCs” or “iPS cells” as used herein refer to pluripotent stem cells generated by reprogramming a somatic cell. iPSCs may be generated by expressing or inducing expression of a combination of factors (“reprogramming factors”). iPS cells may be generated using fetal, postnatal, newborn, juvenile, or adult somatic cells. iPS cells may be obtained from a cell bank. Alternatively, iPS cells may be newly generated (by processes known in the art) prior to commencing differentiation to MSCs or another cell type. The making of iPS cells may be an initial step in the production of differentiated cells. iPS cells may be specifically generated using material from a particular patient or matched donor with the goal of generating tissue-matched MSC cells. iPS cells can be produced from cells that are not substantially immunogenic in an intended recipient, e.g., produced from autologous cells or from cells histocompatible to an intended recipient. As further discussed above (see “pluripotent cells”), pluripotent cells including iPS cells may be genetically modified or otherwise modified to increase longevity, potency, homing, or to deliver a desired factor in cells that are differentiated from such pluripotent cells (for example, MSCs and hemangioblasts).

As a further example, induced pluripotent stem cells may be generated by reprogramming a somatic or other cell by contacting the cell with one or more reprogramming factors. For example, the reprogramming factor(s) may be expressed by the cell, e.g., from an exogenous nucleic acid added to the cell, or from an endogenous gene in response to a factor such as a small molecule, microRNA, or the like that promotes or induces expression of that gene (see Suh and Blelloch, Development 138, 1653-1661 (2011); Miyoshi et al., Cell Stem Cell (2011), doi:10.1016/j.stem.2011.05.001; Sancho-Martinez et al., Journal of Molecular Cell Biology (2011) 1-3; Anokye-Danso et al., Cell Stem Cell 8, 376-388, Apr. 8, 2011; Orkin and Hochedlinger, Cell 145, 835-850, Jun. 10, 2011, each of which is incorporated by reference herein in its entirety). Reprogramming factors may be provided from an exogenous source, e.g., by being added to the culture media, and may be introduced into cells by methods known in the art such as through coupling to cell entry peptides, protein or nucleic acid transfection agents, lipofection, electroporation, biolistic particle delivery system (gene gun), microinjection, and the like. In certain embodiments, factors that can be used to reprogram somatic cells to pluripotent stem cells include, for example, a combination of Oct4 (sometimes referred to as Oct 3/4), Sox2, c-Myc, and Klf4. In other embodiments, factors that can be used to reprogram somatic cells to pluripotent stem cells include, for example, a combination of Oct-4, Sox2, Nanog, and Lin28. In other embodiments, somatic cells are reprogrammed by expressing at least 2 reprogramming factors, at least three reprogramming factors, or four reprogramming factors. In another embodiment, somatic cells are reprogrammed by expressing Oct4, Sox2, MYC, Klf4, Nanog, and Lin28. In other embodiments, additional reprogramming factors are identified and used alone or in combination with one or more known reprogramming factors to reprogram a somatic cell to a pluripotent stem cell. iPS cells typically can be identified by expression of the same markers as embryonic stem cells, though a particular iPS cell line may vary in its expression profile.

The induced pluripotent stem cell may be produced by expressing or inducing the expression of one or more reprogramming factors in a somatic cell. In an embodiment, the somatic cell is a fibroblast, such as a dermal fibroblast, synovial fibroblast, or lung fibroblast, or a non-fibroblastic somatic cell. In an embodiment, the somatic cell is reprogrammed by expressing at least 1, 2, 3, 4, 5 reprogramming factors as described above. In another embodiment, expression of the reprogramming factors may be induced by contacting the somatic cells with at least one agent, such as a small organic molecule agent, that induces expression of reprogramming factors.

The somatic cell may also be reprogrammed using a combinatorial approach wherein the reprogramming factor is expressed (e.g., using a viral vector, plasmid, and the like) and the expression of the reprogramming factor is induced (e.g., using a small organic molecule.) For example, reprogramming factors may be expressed in the somatic cell by infection using a viral vector, such as a retroviral vector or a lentiviral vector. Also, reprogramming factors may be expressed in the somatic cell using a non-integrative vector, such as an episomal plasmid or mRNA. See, e.g., Yu et al., Science. 2009 May 8; 324(5928):797-801, which is hereby incorporated by reference in its entirety. When reprogramming factors are expressed using non-integrative vectors, the factors may be expressed in the cells using electroporation, transfection, or transformation of the somatic cells with the vectors.

Once the reprogramming factors are expressed in the cells, the cells may be cultured by any method known in the art. Over time, cells with ES characteristics appear in the culture dish. The cells may be chosen and subcultured based on, for example, ES morphology, or based on expression of a selectable or detectable marker. The cells may be cultured to produce a culture of cells that resemble ES cells—these are putative iPS cells. iPS cells typically can be identified by expression of the same markers as other embryonic stem cells, though a particular iPS cell line may vary in its expression profile. Exemplary iPS cells may express Oct-4, alkaline phosphatase, SSEA3 surface antigen, SSEA4 surface antigen, TRA160, and/or TRA181.

To confirm the pluripotency of the iPS cells, the cells may be tested in one or more assays of pluripotency. For example, the cells may be tested for expression of ES cell markers; the cells may be evaluated for ability to produce teratomas when transplanted into SCID mice; the cells may be evaluated for ability to differentiate to produce cell types of all three germ layers. Once a pluripotent iPS cell is obtained it may be used to produce hemangioblast and MSC cells.

“Hemangioblasts” or “HBs” as used herein refer to multipotent cells and serve as the common precursor to both hematopoietic and endothelial cell lineages. During embryonic development, they are believed to arise as a transitional cell type that emerges during early mesoderm development and colonizes primitive blood islands (Choi et al. Development 125 (4): 725-732 (1998). Once there, hemangioblasts are capable of giving rise to both primitive and definitive hematopoietic cells, HSCs, and endothelial cells (Mikkola et al, J. Hematother. Stem Cell Res 11(1): 9-17 (2002).

Hemangioblasts may be derived in vitro from both mouse PSCs (Kennedy et al, Nature (386): 488-493 (1997); Perlingeiro et al, Stem Cells (21): 272-280 (2003)) and human PSCs (ref. 14, 15, Yu et al., Blood 2010 116: 4786-4794). Other studies claim to have isolated hemangioblasts from umbilical cord blood (Bordoni et al, Hepatology 45 (5) 1218-1228), circulating CD34− lin− CD45− CD133− cells from peripheral blood (Ciraci et al, Blood 118: 2105-2115), and from mouse uterus (Sun et al, Blood 116 (16): 2932-2941 (2010)). Both mouse and human PSC-derived hemangioblasts have been obtained through the culture and differentiation of clusters of cells grown in liquid culture followed by growth of the cells in semi-solid medium containing various cytokines and growth factors (Kennedy, Perlingeiro, ref 14, 15); see also, U.S. Pat. No. 8,017,393, which is hereby incorporated by reference in its entirety. In an embodiment, hemangioblasts may be generated in vitro from pluripotent stem cells according to the methods described in, for example, U.S. Pat. Nos. 9,938,500; 9,410,123; and WO 2013/082543, all of which are incorporated herein by reference in their entireties. The term hemangioblasts also includes the hemangio-colony forming cells described in U.S. Pat. No. 8,017,393 (incorporated herein by reference in its entirety), which in addition to being capable of differentiating into hematopoietic and endothelial cell lineages, are capable of becoming smooth muscle cells and which are not positive for CD34, CD31, KDR, and CD133. In another embodiment, the hemangioblasts are positive for the blood markers CD43 and CD45 and express low levels or are negative for the pericyte markers CD146, PDGRb, and/or NG2.

Hemangioblasts useful in the methods described herein may be derived or obtained from any of these known methods or any method described herein. For example, embryoid bodies may be formed by culturing pluripotent cells under non-attached conditions, e.g., on a low-adherent substrate, in a “hanging drop”, or through the Able Biott spin bioreactor. In these cultures, PSCs can form clumps or clusters of cells denominated as embryoid bodies. See Itskovitz-Eldor et al., Mol Med. 2000 February; 6(2):88-95, which is hereby incorporated by reference in its entirety. Typically, embryoid bodies initially form as solid clumps or clusters of pluripotent cells, and over time some of the embryoid bodies come to include fluid filled cavities, the latter former being referred to in the literature as “simple” EBs and the latter as “cystic” embryoid bodies. Id. The cells in these EBs (both solid and cystic forms) can differentiate and over time produce increasing numbers of cells. Optionally EBs may then be cultured as adherent cultures and allowed to form outgrowths. Likewise, pluripotent cells that are allowed to overgrow and form a multilayer cell population can differentiate over time.

In one embodiment, hemangioblasts are generated by the steps comprising (a) culturing a PSC line for 2, 3, 4, 5, 6 or 7 days to form clusters of cells (embryoid bodies; EBs), and (b) inducing said clusters of cells or EBs to differentiate into hemangioblasts. In a further embodiment, the clusters of cells or EBs in step (b) of are cultured in a cytokine-rich serum-free methylcellulose based medium. In an embodiment, hemangioblasts are generated by inducing differentiation of any pluripotent cell as described herein.

In one embodiment, the clusters of cells or EBs are cultured for at least 4, 5, 6, 7, 8, 9, 10, 11, 12, 13, 14, 15, 16, 17, 18, 19, or 20 days in culture in a serum free methylcellulose medium comprising one or more ingredients selected from the group comprising penicillin/streptomycin (pen/strp), EX-CYTE® growth supplement (a water-soluble concentrate comprising 9.0-11.0 g/L cholesterol and 13.0-18.0 g/L lipoproteins and fatty acids at pH 7-8.4), Flt3-ligand (FL), vascular endothelial growth factor (VEGF), thrombopoietin (TPO), basic fibroblast growth factor (bFGF), stem cell derived factor (SCF), granulocyte macrophage colony stimulating factor (GM-CSF), interleukin 3 (IL3), and interleukin 6 (IL6), and producing hemangioblasts. In a preferred embodiment of the instant presently disclosed subject matter, hemangioblasts are harvested between 6-14 days, of being cultured in, for example, serum-free methylcellulose plus one or more of the ingredients of the previous embodiment. In a preferred embodiment, the one or more ingredients may be present in said medium at the following concentrations: Flt3-ligand (FL) at 50 ng/ml, vascular endothelial growth factor (VEGF) at 50 ng/ml, thrombopoietin (TPO) at 50 ng/ml, and basic fibroblast growth factor (bFGF) at 20-30 ng/ml, 50 ng/ml stem cell derived factor (SCF), 20 ng/ml granulocyte macrophage colony stimulating factor (GM-CSF), 20 ng/ml interleukin 3 (IL3), and 20 ng/ml interleukin 6 (IL6).

In vitro Generation of Mesenchymal Stem Cells

An embodiment of the instant presently disclosed subject matter comprises methods of producing mesenchymal stem cells (hereinafter, “HMCs”) by in vitro differentiation of hemangioblasts. The hemangioblasts may be obtained by any of the methods described herein. In an embodiment, the hemangioblasts are obtained by in vitro differentiation of pluripotent stem cells. Pluripotent stem cells can be cultured on feeders (e.g., human dermal fibroblasts, or mouse embryonic fibroblasts), or in feeder-free conditions. In some embodiments, hemangioblasts are cultured in feeder-free conditions then plated on an extracellular matrix. In another embodiment, said extracellular matrix is selected from the group consisting of laminin, fibronectin, vitronectin, proteoglycan, entactin, collagen, collagen I, collagen IV, heparan sulfate, a soluble preparation from Engelbreth-Holm-Swarm (EHS) mouse sarcoma cells, Matrigel, and a human basement membrane extract. In a still further embodiment, said extracellular matrix may be derived from any mammalian, including human, origin.

In another embodiment, hemangioblasts are re-plated and cultured for at least 8, 9, 10, 11, 12, 13, 14, 15, 16, 17, 18, 19, 20, 21, 22, 23, 24, 25, 26, 27, 28, 29, 30, 31, 32, 33, 34, 35, or 36 days forming a preparation of HMCs. In an embodiment, initial plating of hemangioblasts onto substrate-coated tissue culture dishes may be done at a concentration of about 50,000 to about 100,000 cells/cm². During culturing of hemangioblasts, a portion of hemangioblasts adheres to the culture plate and begins to differentiate into HMCs. Adherent cells are passaged every 3-6 days or more than 6 days, e.g., about 6-10 days, or about 10-15 days, depending on their growth rate, plating density, and perceived degree of confluence. For passaging, harvest density may be about 5,000 to about 20,000 cells/cm², or about 20,000 to about 40,000 cells/cm². After the cells are harvested, cells are counted and may be replated at a density of between about 2500 to about 6000 cells/cm². In one embodiment, HMCs are generated by the steps comprising (a) culturing ESCs for 8-12 days and producing hemangioblasts, (b) harvesting hemangioblasts, (c) re-plating the hemangioblasts of step (b), and (d) culturing the hemangioblasts of step (c) for between 14-30 days.

In one embodiment, the hemangioblasts are harvested, re-plated and cultured in liquid medium under feeder-free conditions wherein no feeder layer of cells such as mouse embryonic fibroblasts, OP9 cells, or other cell types known to one of ordinary skill in the art are contained in the culture. In a preferred embodiment, hemangioblasts are cultured on an extracellular matrix. In a further preferred embodiment, hemangioblasts are cultured on an extracellular matrix, wherein said matrix comprises a soluble preparation from Engelbreth-Holm-Swarm (EHS) mouse sarcoma cells that gels at room temperature to form a reconstituted basement membrane (Matrigel). In a still further preferred embodiment, hemangioblasts are formed according to the steps comprising (a) culturing said hemangioblasts on an extracellular matrix for at least 7 days, (b) transferring the hemangioblasts of step (a) to non-coated tissue culture plate and further culturing said hemangioblasts of step (b) for between about 7 to 14 days. The hemangioblasts may be cultured in the presence of one or more of the factors selected from the group consisting of: transforming growth factor beta (TGF-beta), epidermal growth factor (EGF), insulin-like growth factor 1, bovine fibroblast growth factor (bFGF), and/or platelet-derived growth factor (PDGF). In an embodiment, the extracellular matrix is selected from the group consisting of Human Basement Membrane Extract (BME) (e.g., Cultrex BME, Trevigen) or an EHS matrix, laminin, fibronectin, vitronectin, proteoglycan, entactin, collagen (e.g., collagen I, collagen IV), and heparan sulfate. Said extracellular matrix or matrix components may be of mammalian, or more specifically human, origin. In one embodiment, hemangioblasts are cultured in a liquid medium comprising serum on an extracellular matrix protein-coated plate, wherein the culture medium may comprise ingredients selected from αMEM (Sigma-Aldrich) supplemented with 10-20% fetal calf serum (αMEM+20% FCS), αMEM supplemented with 10-20% heat-inactivated human AB serum, and IMDM supplemented with 10-20% heat inactivated AB human serum.

In another embodiment, hemangioblasts are cultured in a medium comprising serum or a serum replacement, such as αMEM supplemented with 20% fetal calf serum. In another embodiment, hemangioblasts are cultured in a serum-free medium.

In a further embodiment, hemangioblasts are cultured on an extracellular matrix for about 9, 10, 11, 12, 13, 14, 15, 16, 17, 18, 19, 20, 21, 22, 23, 24, 25, 26, 27, 28, 29 or 30 days. In a still further embodiment of the instant presently disclosed subject matter, HMCs are generated by the steps comprising (a) culturing hemangioblasts on an extracellular matrix for about 7 days, (b) transferring the hemangioblasts of step (a) to an uncoated tissue culture dish and culturing the hemangioblasts for an additional 9-100 days, about 9, 10, 11, 12, 13,14, 15, 16, 17, 18, 19, 20, 21, 22, 23, 24, 25, 30, 35, 40, 50, 60, 70, 80, 90 or 100 days. In yet another embodiment, HMCs are generated by the steps comprising (a) culturing hemangioblasts on an extracellular matrix for about 7 days, (b) transferring the hemangioblasts of step (a) to a coated tissue culture dish and culturing the hemangioblasts for an additional 9-100 days, about 9, 10, 11, 12, 13,14, 15, 16, 17, 18, 19, 20, 21, 22, 23, 24, 25, 30, 35, 40, 50, 60, 70, 80, 90 or 100 days.

In an embodiment of the instant presently disclosed subject matter, hemangioblasts are differentiated from PSCs by following the steps comprising: (a) culturing PSCs in the presence of vascular endothelial growth factor (VEGF) and/or bone morphogenic protein 4 (BMP-4) (by way of non-limiting examples) to form clusters of cells or EBs; (b) culturing said clusters of cells or EBs in the presence of at least one growth factor (e.g., basic fibroblast growth factor (bFGF), vascular endothelial growth factor (VEGF), bone morphogenic protein 4 (BMP-4), stem cell factor (SCF), Flt 3L (FL), thrombopoietin (TPO), and/or tPTD-HOXB4) in an amount sufficient to induce the differentiation of said clusters of cells or EBs into hemangioblasts; and (c) culturing said hemangioblasts in a medium comprising at least one additional growth factor (e.g., insulin, transferrin, granulocyte macrophage colony-stimulating factor (GM-CSF), interleukin-3 (IL-3), interleukin-6 (IL-6), granulocyte colony-stimulating factor (G-CSF), erythropoietin (EPO), stem cell factor (SCF), vascular endothelial growth factor (VEGF), bone morphogenic protein 4 (BMP-4), and/or tPTD-HOXB4), wherein said at least one additional growth factor is provided in an amount sufficient to expand said clusters of cells in said culture, and wherein copper is optionally added to any of the steps (a)-(c).

In an embodiment of the instant presently disclosed subject matter, HMCs are generated by culturing hemangioblasts, wherein said hemangioblasts are differentiated from PSCs by following the steps comprising: (a) culturing PSCs in the presence of vascular endothelial growth factor (VEGF) and bone morphogenic protein 4 (BMP-4) within 0-48 hours of initiation of said culture to form clusters of cells or EBs; (b) culturing said clusters of cells or EBs in the presence of at least one growth factor selected from the group comprising basic fibroblast growth factor (bFGF), vascular endothelial growth factor (VEGF), bone morphogenic protein 4 (BMP-4), stem cell factor (SCF), Flt 3L (FL), thrombopoietin (TPO), and tPTD-HOXB4 in an amount sufficient to induce the differentiation of said clusters of cells or EBs into hemangioblasts; and (c) culturing said hemangioblasts in a medium comprising at least one additional growth factor selected from the group consisting of insulin, transferrin, granulocyte macrophage colony-stimulating factor (GM-CSF), interleukin-3 (IL-3), interleukin-6 (IL-6), granulocyte colony-stimulating factor (G-CSF), erythropoietin (EPO), stem cell factor (SCF), vascular endothelial growth factor (VEGF), bone morphogenic protein 4 (BMP-4), and tPTD-HOXB4, wherein said at least one additional growth factor is provided in an amount sufficient to expand hemangioblasts in said culture.

In another embodiment, HMCs are generated by the steps comprising: (a) harvesting hemangioblasts after at least 6, 7, 8, 9, 10, 11, 12, 13, or 14 days of inducing PSCs to differentiate into said hemangioblasts, and (b) harvesting HMCs that are generated within about 25, 26, 27, 28, 29, 30, 31, 32, 33, 34, 35, 36, 37, 38, 39, 40, 41, 42, 43, 44, 45, 46, 47, 48, 49 or 50 days of inducing said hemangioblasts from step (a) to differentiate into said mesenchymal cells.

In yet another embodiment, a preparation of at least 80, 85, 90, 95, 100, 125 or 125 million HMCs are generated from about 200,000 hemangioblasts within about 26, 27, 28, 29, 30, 31, 32, 33, 34, or 35 days of culturing the hemangioblasts, wherein said preparation of HMCs comprises less than about 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% human embryonic stem cells. In still another embodiment, at least 80, 85, 90, 100, 125 or 150 million HMCs are generated from about 200,000 hemangioblasts within about 26, 27, 28, 29, 30, 31, 32, 33, 34, or 35 days of culturing the hemangioblasts.

Extracellular Vesicles Secreted from Mesenchymal Stem Cells

The presently disclosed subject matter also provides extracellular vesicles isolated, derived, secreted, or released from a cell, e.g., the HMCs of the presently disclosed subject matter.

As used herein, the term “extracellular vesicle” or “EV” refers to lipid bound vesicles secreted by cells into the extracellular space. The three main subtypes of EVs are microvesicles (MVs), exosomes, and apoptotic bodies, which are differentiated based upon their biogenesis, release pathways, size, content, and function (Zaborowski M. P., et al. Bioscience. 2015; 65:783-797). Generally extracellular vesicles range in diameter from 20 nm to 5000 nm, and can comprise various macromolecular payload either within the internal space (i.e., lumen), displayed on the external surface of the extracellular vesicle, and/or spanning the membrane. Said payload can comprise nucleic acids, e.g., microRNAs (miRNA), long non-coding RNAs (lncRNA), mRNAs, DNA fragments; proteins, carbohydrates, lipids, small molecules, and/or combinations thereof. By way of example and without limitation, extracellular vesicles include apoptotic bodies, fragments of cells, vesicles derived/secreted from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived/secreted from a living or dead organism, explanted tissues or organs, prokaryotic or eukaryotic cells, and/or cultured cells.

As used herein, the term “exosome” refers to a cell-derived small vesicle comprising a membrane that encloses an internal space (i.e., lumen), and which is formed from said cell by direct plasma membrane budding or by fusion of the late endosome with the plasma membrane (Yáñez-Mó M., et al. J. Extracell. Vesicles. 2015; 4:27066). Specifically, exosomes are involved in protein sorting, recycling, storage, transport, and release. Exosomes are generally between 20-300 nm in diameter. Exosomes are secreted by all cell types and have been found in plasma, urine, semen, saliva, bronchial fluid, cerebral spinal fluid (CSF), breast milk, serum, amniotic fluid, synovial fluid, tears, lymph, bile, and gastric acid.

Exosomes have been found to participate in cell-cell communication, cell maintenance, and tumor progression. In addition, exosomes have been found to stimulate immune responses by acting as antigen-presenting vesicles (Bobrie A., et al., Traffic. 2011; 12:1659-1668). In the nervous system, exosomes haven been found to help promote myelin formation, neurite growth, and neuronal survival, thus playing a role in tissue repair and regeneration (Faure J., et al. Mol. Cell. Neurosci. 2006; 31:642-648). At the same time, exosomes in the central nervous system (CNS) have been found to contain pathogenic proteins, such as beta amyloid peptide, superoxide dismutase, and alpha synuclein that may aid in disease progression (Fevrier B., et al., Proc. Natl. Acad. Sci. USA. 2004; 101:9683-9688). Exosomes have also been shown as carriers for disease markers. The use of exosomes as carriers of biomarkers is ideal because these vesicles are found in bodily fluids, such as blood and urine, which allows for minimally to non-invasive “liquid biopsy” type methods to diagnose and even monitor a patient's response to treatment.

In addition to their natural role in cell-cell interactions, exosomes can be loaded with different cargos, e.g., drugs and exogenous nucleic acids or proteins, and deliver this cargo to different cells. The cargo can be conjugated to an extracellular vesicle, embedded within an extracellular vesicle, encapsulated within an extracellular vesicle, or otherwise carried by an extracellular vesicle, or any combination thereof. Thus, as used herein, a reference to a cargo being “present” in an extracellular vesicle or its lumen is understood to include any of the foregoing means of carrying the cargo.

A cargo can be an endogenous cargo, an exogenous cargo, or a combination thereof. Examples of cargos that can be conjugated, embedded, encapsulated within or otherwise carried by an extracellular vesicle described herein include, without limitation, nucleic acid molecules (e.g., DNA, cDNA, antisense oligonucleotides, mRNA, inhibitory RNAs (e.g., antisense RNAs, miRNAs, small interfering RNAs (siRNAs), short hairpin RNAs (shRNAs), and agomiRs), antagomiRs, primary miRNAs (pri-miRNAs), long non-coding RNAs (lncRNAs), small nuclear RNA (snRNA), small nucleolar RNA (snoRNA), and microbial RNAs), polypeptides (e.g., enzymes, antibodies), lipids, hormones, vitamins, minerals, small molecules, and pharmaceuticals, or any combination thereof. Importantly, exosomes, are natural carriers for miRNAs and other non-coding RNAs, and the direct membrane fusion with the target cell allows contents to be delivered directly into the cytosol. This makes exosomes an excellent delivery system for small molecules (Lai R. C., et al. Biotechnol. Adv. 2013; 31:543-551).

Microvesicles are EVs that form by direct outward budding, or pinching, of the cell's plasma membrane. The size of microvesicles typically range from 100 nm up to 1000 nm in diameter. The route of microvesicles formation is not well understood, however, it is thought to require cytoskeleton components, such as actin and microtubules, along with molecular motors (kinesins and myosins), and fusion machinery (SNAREs and tethering factors) (Cai H., et al. Dev. Cell. 2007; 12:671-682). The number of microvesicles produced depends on the donor cell's physiological state and microenvironment (Zaborowski M. P., et al. Bioscience. 2015; 65:783-797). Likewise, it has been previously demonstrated that the number of microvesicles consumed depends on the physiological state and microenvironment of recipient cells. Like exosomes, microvesicles are involved in cell-cell communication between local and distant cells. The ability of these EVs to alter the recipient cell has been well demonstrated (Harding C. V., et al., J. Cell Biol. 2013; 200:367-371; White I. J., et al., EMBO J. 2006; 25:1-12). The uniqueness of EVs is that they have the ability to package active cargo (proteins, nucleic acids, and lipids) and deliver it to another cell, neighboring or distant, and alter the recipient cell's functions with its delivery.

Apoptotic bodies are released by dying cells into the extracellular space. They are reported to range in size from 50 nm up to 5000 nm in diameter, with the size of most apoptotic bodies tending to be on the larger side (Borges F., et al. Braz. J. Med. Biol. Res. 2013; 46:824-830). These bodies form by a separation of the cell's plasma membrane from the cytoskeleton as a result of increased hydrostatic pressure after the cell contracts (Wickman G., et al. Cell Death Differ. 2012; 19:735-742). The composition of apoptotic bodies is in direct contrast with exosomes and microvesicles. Unlike exosomes and microvesicles, apoptotic bodies contain intact organelles, chromatin, and small amounts of glycosylated proteins (Borges F., et al., Braz. J. Med. Biol. Res. 2013; 46:824-830; Thery C., et al. J. Immunol. 2001; 166:7309-7318).

### Methods for Isolating Extracellular Vesicles

The EVs of the presently disclosed subject matter can be isolated, secreted, derived, or separated, from a medium or other source material, e.g., the HMCs of the presently disclosed subject matter, using routine methods known in the art (see, for example the techniques described in Taylor et al., Serum/Plasma Proteomics, Chapter 15, “Extracellular vesicle Isolation for Proteomic Analyses and RNA Profiling,” Springer Science, 2011; and Tauro et al., Methods 56 (2012) 293-304, and references cited therein) and as described in the Examples section below. The most commonly used method involves multiple centrifugation and ultracentrifugation steps.

Physical properties of EVs (e.g., HMC-EVs) may be employed for EV isolation, purification or enrichment, including separation on the basis of electrical charge (e.g., electrophoretic separation), size (e.g., filtration, molecular sieving, etc), density (e.g., regular or gradient centrifugation), Svedberg constant (e.g., sedimentation with or without external force, etc). Alternatively, or additionally, isolation may be based on one or more biological properties, and include methods that may employ surface markers (e.g., for precipitation, reversible binding to solid phase, FACS separation, specific ligand binding, non-specific ligand binding, immuno-magnetic capture of EVs using magnetic beads coated with antibodies directed against proteins exposed on EV membranes, etc.).

Methods based on the use of volume-excluding polymers, such as PEG, have been recently described by a number of different groups (U.S. Pat. Appl. 20130273544, U.S. Pat. Appl. 20130337440). Two such products are ExoQuick (System Biosciences, Mountain View, USA) and Total Exosome Isolation Reagent (Life Technologies, Carlsbad, USA). These polymers work by tying up water molecules and forcing less-soluble components such as extracellular vesicles, as well as proteins out of solution, allowing them to be collected by a short, low-speed centrifugation.

In some embodiments, isolation, purification, and enrichment can be done in a general and non-selective manner (typically including serial centrifugation). Alternatively, isolation, purification, and enrichment can be done in a more specific and selective manner (e.g., using producer cell-specific surface markers). For example, specific surface markers may be used in immunoprecipitation, FACS sorting, affinity purification, or bead-bound ligands for magnetic separation.

In some embodiments, tangential flow filtration may be used to isolate or purify the EVs (e.g., HMC-EVs).

In some embodiments, size exclusion chromatography can be utilized to isolate or purify the EVs (e.g., HMC-EVs). Size exclusion chromatography techniques are known in the art. In some embodiments, density gradient centrifugation can be utilized to isolate the EVs. In some embodiments, the isolation of EVs (e.g., HMC-EVs) may involve ion chromatography, such as anion exchange, cation exchange, or mixed mode chromatography. In some embodiments, the isolation of EVs (e.g., HMC-EVs) may involve desalting, dialysis, tangential flow filtration, ultrafiltration, or diafiltration, or any combination thereof. In some embodiments, the isolation of EVs (e.g., HMC-EVs) may involve combinations of methods that include, but are not limited to, differential centrifugation, size-based membrane filtration, concentration and/or rate zonal centrifugation. In some embodiments, the isolation of EVs (e.g., HMC-EVs) may involve one or more centrifugation steps. The centrifugation may be performed at about 50,000 to 150,000-g. The centrifugation may be performed at about 50,000×g, 75,000×g, 100,000×g, 125,000×g, or 150,000×g. In another embodiment, EVs (e.g., HMC-EVs) are separated from nonmembranous particles, using their relatively low buoyant density (Raposo et al., 1996; Escola et al., 1998; van Niel et al., 2003; Wubbolts et al., 2003). Kits for such isolation are commercially available, for example, from Qiagen, InVitrogen and SBI. Methods for loading EVs with a therapeutic agent are known in the art and include lipofection, electroporation, as well as any standard transfection method.

In some embodiments, the presently disclosed subject matter provides methods for isolating HMC-EVs secreted from HMCs obtained by in vitro differentiation of pluripotent stem cells. The method comprises providing HMCs obtained by in vitro differentiation of pluripotent stem cells, and isolating extracellular vesicles. The HMC-EVs may be isolated by any method known in the art or as described herein. In some embodiments, the HMC-EVs are isolated by tangential flow filtration. In some embodiments, the HMC-EVs are isolated by ultracentrifugation. In some embodiments, the HMC-EVs are isolated by cation exchange chromatography. In some embodiments, the HMC-EVs are isolated by anion exchange chromatography.

Characteristics and Compositions of HMCs and/HMC-EVs

The presently disclosed subject matter further provides compositions comprising HMCs obtained by in vitro differentiation of pluripotent stem cells, and/or extracellular vesicles secreted from the HMCs (HMC-EVs) of the presently disclosed subject matter. In an embodiment, the HMCs are obtained by in vitro differentiation of hemangioblasts. Expression levels of certain phenotypic markers may be determined by any method known in the art, such as immunohistochemistry. Expression of certain genes may be determined by any method known in the art, such as RT-PCR and RNA-Seq.

In an embodiment, the HMCs of the presently disclosed subject matter express at least 2, at least 3, at least 4, at least 5, at least 6, at least 7 or at least 8 markers selected from the group comprising CD9, CD13, CD29, CD44, CD73, CD90, CD105, CD166, and HLA-ABC. A still further embodiment, the HMCs of the presently disclosed subject matter express at least 2, at least 3, at least 4, at least 5 or at least 6 markers selected from the group consisting of CD9, CD13, CD29, CD44, CD73, CD90 and CD105, and wherein said HMCs s do not express CD2, CD3, CD4, CD5, CD7, CD8, CD14, CD15, CD16, CD19, CD20, CD22, CD33, CD36, CD38, CD61, CD62E and CD133. In another embodiment, the HMCs of the presently disclosed subject matter express at least 1, at least 2, at least 3, at least 4, at least 5 or at least 6 markers selected from the group consisting of AIRE-1, IL-11, CD10, CD24, ANG-1, and CXCL1.

In an embodiment, the composition comprises HMCs, wherein about 50%, 55%, 60%, 65%, 70%, 75%, 80%, 85%, 90%, 95%, 98%, 99%, or 100% of the HMCs express CD9, CD13, CD29, CD44, CD73, CD90, CD105, CD166, and HLA-abc after about 15, 16, 17, 18, 19, 20, 21, 22, 23, 24, 25, 26, 27, 28, 29, or 30 days in culture. In an embodiment of the instant presently disclosed subject matter at least 50%, 55%, 60%, 65%, 70%, 75%, 80%, 85%, 90%, 95%, 98%, 99%, or 100% of the HMCs in a composition of the presently disclosed subject matter express at least 2, at least 3, at least 4, at least 5, at least 6, at least 7 or at least 8 markers selected from the group consisting of CD9, CD13, CD29, CD44, CD73, CD90, CD105, CD166, and HLA-ABC and lack expression of CD2, CD3, CD4, CD5, CD7, CD8, CD14, CD15, CD16, CD19, CD20, CD22, CD33, CD36, CD38, CD61, CD62E, CD133 and Stro-1 after about 15, 16, 17, 18, 19, 20, 21, 22, 23, 24, 25, 26, 27, 28, 29, or 30 days in culture. The HMCs in a composition of the presently disclosed subject matter may further express at least 1, at least 2, at least 3, at least 4, at least 5 or at least 6 markers selected from the group consisting of AIRE-1, IL-11, CD10, CD24, ANG-1, and CXCL1.

In an embodiment, the composition comprises HMCs, wherein at least 30% of the HMCs are positive for CD10. Additionally, at least 60% of the HMCs may be positive for markers CD73, CD90, CD105, CD13, CD29, CD44, and CD166 and HLA-ABC. In an exemplary embodiment, less than 30% of the HMCs may be positive for markers CD31, CD34, CD45, CD133, FGFR2, CD271, Stro-1, CXCR4 and TLR3.

In another embodiment, the composition comprises HMCs, wherein at least 50% of the HMCs are positive for CD105 or CD73 within about 7-20 (e.g., 15) days of culture. In a preferred embodiment of the instant presently disclosed subject matter, at least 50% of the HMCs are positive for CD105 or CD73 after about 7-15 days of culture. In a further embodiment of the instant presently disclosed subject matter, at least 80% of the HMCs are positive for CD105 and CD73 within about 20 days of culture. In still a further embodiment of the instant presently disclosed subject matter, at least 80% of a composition of HMCs are positive for CD105 and CD73 within about 20 days of culture.

In an embodiment, the composition comprises HMCs, wherein at least 20%, 30%, 40%, or 50% of said HMCs may be positive for (i) at least one of CD10, CD24, IL-11, AIRE-1, ANG-1, CXCL1, CD105, CD73 and CD90; (ii) at least one of CD10, CD24, IL-11, AIRE-1, ANG-1, CXCL1, CD105, CD73, CD90, CD105, CD13, CD29, CD44, CD166, CD274, and HLA-ABC; (iii) CD105, CD73 and/or CD90 or (iv) any combination thereof. At least 20%, 30%, 40%, or 50% of said HMCs may be positive for (i) at least two of CD105, CD73 and/or CD90 (ii) at least two of CD10, CD24, IL-11, AIRE-1, ANG-1, CXCL1, CD105, CD73 and CD90; or (iii) all of CD10, CD24, IL-11, AIRE-1, ANG-1, CXCL1, CD105, CD73, CD90, CD105, CD13, CD29, CD44, CD166, CD274, and HLA-ABC. At least 20%, 30%, 40%, or 50% of said HMCs (i) may be positive for CD105, CD73 and CD90; (ii) positive for CD10, CD24, IL-11, AIRE-1, ANG-1, CXCL1, CD105, CD73, CD90, CD105, CD13, CD29, CD 44, CD166, CD274, and HLA-ABC and/or (ii) may be negative for or less than 5% or less than 10% of the cells express CD31, 34, 45, 133, FGFR2, CD271, Stro-1, CXCR4, and/or TLR3. At least 20%, 30%, 40%, 50%, 60%, 70%, 80% or 90% of said HMCs may be positive for (i) one or more of CD105, CD73 and CD90 (ii) one or more of CD10, CD24, IL-11, AIRE-1, ANG-1, CXCL1, CD105, CD73 and CD90; or (iii) one or more of CD10, CD24, IL-11, AIRE-1, ANG-1, CXCL1, CD105, CD73, CD90, CD105, CD13, CD29, CD 44, CD166, CD274, and HLA-ABC.

In another embodiment, the composition comprises HMCs, wherein at least 20%, 30%, 40%, or 50% of said HMCs (i) may be positive for all of CD10, CD24, IL-11, AIRE-1, ANG-1, CXCL1, CD105, CD73, CD90, CD105, CD13, CD29, CD 44, CD166, CD274, and HLA-ABC and (ii) may be negative for or less than 5% or less than 10% of the cells express CD31, 34, 45, 133, FGFR2, CD271, Stro-1, CXCR4 and/or TLR3.

In a further embodiment, the composition comprises HMCs, wherein at least 20%, 30%, 40%, or 50% of said HMCs may be positive for (i) all of CD10, CD24, IL-11, AIRE-1, ANG-1, CXCL1, CD105, CD73 and CD90; or (ii) all of CD73, CD90, CD105, CD13, CD29, CD44, CD166, CD274, and HLA-ABC.

In yet another embodiment, the composition comprises HMCs, wherein at least 20%, 30%, 40%, 50%, 60%, 70%, 80% or 90% of said HMCs may be positive for (i) at least one of CD10, CD24, IL-11, AIRE-1, ANG-1, CXCL1, CD105, CD73 and CD90; or (ii) at least one of CD73, CD90, CD105, CD13, CD29, CD 44, CD166, CD274, and HLA-ABC.

In another embodiment, the HMCs may not express or less than 5% or less than 10% of the HMCs may express at least one of CD31, 34, 45, 133, FGFR2, CD271, Stro-1, CXCR4, or TLR3.

In addition to the characteristics described above, the HMCs of the presently disclosed subject matter may possess phenotypes of younger cells as compared to adult-derived MSCs. In one embodiment, the HMCs are capable of undergoing at least or about 5, 10, 15, 20, 25, 30, 35, 40, 45, 50, 55, 60, or more population doublings in culture. In contrast, adult-derived MSCs typically undergo 2-3 doublings in culture. In another embodiment, the HMCs of the presently disclosed subject matter have longer telomere lengths, greater immunosuppressive effects, fewer vacuoles, divide faster, divide more readily in culture, higher CD90 expression, are less lineage committed, or combinations thereof, compared to adult-derived MSCs. In another embodiment, the HMCs of the presently disclosed subject matter have increased expression of transcripts promoting cell proliferation (i.e., have a higher proliferative capacity) and reduced expression of transcripts involved in terminal cell differentiation compared to adult-derived MSCs.

In an embodiment, the HMCs are “early passage” HMCs and may be passaged no more than 1, 2, 3, 4, 5, 6, 7, or 8 times. In an embodiment, early passage HMCs are passaged no more than 4 times. In another embodiment, the early passage HMCs are passaged no more than 5 times. In another embodiment, the early passage HMCs are passaged no more than 6 times. In addition to the HMCs characteristics described above, early passage HMCs may, in a resting or basal state, express mRNA encoding interleukin-6 (IL-6) at a level which may be less than ten percent of the IL-6 mRNA level expressed by BM-MSCs or AD-MSCs in a resting or basal state. VEGF mRNA levels may also be downregulated in early passage HMCs, in a resting or basal state, compared to BM-MSCs in a resting or basal state. In another embodiment, the HMCs may, in a resting or basal state, express mRNA encoding CD24 at a level that is greater than the CD24 mRNA level expressed by BM-MSC or AD-MSC preparations in a resting or basal state. Other mRNA levels that may be upregulated in early passage HMCs, in a resting or basal state, compared to BM-MSCs, in a resting or basal state, include AIRE, ANGPT1 (ANG-1), CXCL1, CD10, and IL-11. Additionally, the early passage HMCs, in a resting or basal state, may be negative for one or more of mRNAs encoding ANGPT2, CD31, CD34, CD45, HLA-G, IL2RA, IL3, IL12B.

In a further embodiment, the early passage HMCs express one or more markers selected from the group consisting of CD13, CD29, CD44, CD73, CD90, CD105, CD166, and HLA-ABC, as determined by immunohistochemistry. In another embodiment, the early passage HMCs are negative for one or more markers selected from the group consisting of CD31, CD34, CD45, CXCR4, HLA-DR, FGFR2, TLR3, CD106, CD133, and CD271, as determined by immunohistochemistry.

In an embodiment, expression levels of CD10 is upregulated in early passage HMCs compared with the expression levels of CD10 in BM-MSCs, as determined by immunohistochemistry. In another embodiment, expression levels of CD10 in early passage HMCs may be about the same the expression levels of CD10 in BM-MSCs. In another embodiment, expression levels of Stro-1 is downregulated in early passage HMCs of the presently disclosed subject matter compared with the expression levels of Stro-1 in BM-MSCs, as determined by immunohistochemistry. In a specific embodiment, a composition comprises early passage HMCs, wherein about 5-10% of the early passage HMCs express Stro-1.

In a further embodiment, the HMCs of the presently disclosed subject matter express higher levels of certain genes compared to BM-MSCs, UCB-MSCs, or AD-MSCs. For example, the HMCs of the presently disclosed subject matter may express higher levels of any of the genes listed in Table 3 compared to BM-MSCs, and/or any of the genes listed in Table 5 compared to UCB-MSCs, and/or any of the genes listed in Table 7 compared to AD-MSCs. In another embodiment, the HMCs of the presently disclosed subject matter may express lower levels of any of the genes listed in Table 4 compared to BM-MSCs, and/or any of the genes listed in Table 6 compared to UCB-MSCs, and/or any of the genes listed in Table 8 compared to AD-MSCs.

In an embodiment, genes associated with increased migration and chemotaxis, such as MMP9 is expressed at a higher level in the HMCs of the presently disclosed subject matter compared to BM-MSCs or UCB-MSCs. In another embodiment, Lgr5, a marker of multipotent stem cells, is expressed at a higher level in the HMCs of the presently disclosed subject matter compared to BM-MSCs or UCB-MSCs. In a further embodiment, CD24 is expressed at a higher level in the HMCs of the presently disclosed subject matter compared to BM-MSCs and IL-6 is expressed at a lower level in the MSCs of the presently disclosed subject matter compared to BM-MSCs. In yet another embodiment, neuro-related genes, such as NGF, NTF-4, NTRK-2, NTRK-3, and DCC (Netrin-1), are expressed at a higher level in the HMCs of the presently disclosed subject matter compared to BM-MSCs or UCB-MSCs. MSCs of the presently disclosed subject matter may be selected or purified based on any of the genes that are differentially expressed.

In some embodiments, the HMCs of the presently disclosed subject matter may express lower levels of any of the miRNA listed in Table 21 compared to HMC-EVs. In some embodiments, the HMCs of the presently disclosed subject matter may express higher levels of any of the miRNA listed in Table 22 compared to HMC-EVs.

In a further embodiment, the HMC-EVs of the presently disclosed subject matter express higher levels of certain miRNA, genes, or proteins compared to BM-MSCs-EVs, UCB-MSCs-EVs, or AD-MSCs-EVs.

In some embodiments, the HMC-EVs of the presently disclosed subject matter may express higher levels of any of the miRNAs listed in Table 9 compared to UCB-MSCs-EVS, and/or any of the miRNAs listed in Table 11 compared to BM-MSC-EVs, and/or any of the miRNAs listed in Table 13 compared to AD-MSC-EVs. In another embodiment, the HMC-EVs of the presently disclosed subject matter may express lower levels of any of the miRNAs listed in Table 10 compared to UCB-MSCs-EVS, and/or any of the miRNAs listed in Table 12 compared to BM-MSC-EVs, and/or any of the miRNAs listed in Table 13 compared to AD-MSC-EVs. In some embodiments, the HMC-EVs of the presently disclosed subject matter may express higher levels of any of the proteins listed in Table 15 compared to UCB-MSCs-EVS, and/or any of the proteins listed in Table 17 compared to BM-MSC-EVs, and/or any of the miRNA listed in Table 19 compared to AD-MSC-EVs. In another embodiment, the HMC-EVs of the presently disclosed subject matter may express lower levels of any of the proteins listed in Table 16 compared to UCB-MSCs-EVS, and/or any of the proteins listed in Table 18 compared to BM-MSC-EVs, and/or any of the proteins listed in Table 20 compared to AD-MSC-EVs.

In some embodiments, the HMC-EVs express at least one of the miRNAs selected from the group consisting of hsa-miR-125b-5p, hsa-miR-181a-5p, hsa-miR-199b-5p, hsa-miR-21-5p, hsa-miR-23a-3p, hsa-miR-125a-5p, hsa-miR-106a-5p+hsa-miR-17-5p and hsa-miR-221-3p at a higher level compared to BM-MSC-EVs, UCB-MSC-EVs, and/or AD-MSC-EVs.

In some embodiments, the HMC-EVs express at least one of the proteins selected from the group consisting of ALDOC, ANXA5, APBB2, BASP1, CAV1, CD81, CD99, CKM, EPB41L3, FDPS, GNAQ, GNG12, GP9, H2AC20, H2AC21, H3-3A, H3-7, H4-16, HLA-A, ITGA2, KPNA2, KRAS, KRT4, LRRC59, MAMDC2, MARCKSL1, MDGA1, MERTK, MFGE8, MMP14, MVP, PCDH1, PDGFRB, PDIA3, RPL13, RPS18, RPS3A, RPS4X, SDCBP, SLC2A1, SLC3A2, TAGLN2, TNC, TSPAN14, TSPAN33, TSPAN9, TTYH3, UCHL1, VAT1, YWHAB, and YWHAQ at a higher level compared to BM-MSC-EVs, UCB-MSC-EVs, and/or AD-MSC-EVs.

In some embodiments, the HMC-EVs express at least one of the proteins selected from the group consisting of ADGRG6, AGRN, ANXA6, APOC4, ARHGAP1, ARGHDIA, ARL8A, ARPC5, B2M, BBS1, BLVRA, BST1, CA2, CCN2, CCNB3, CD34, CD36, CD47, CORO1A, DTD1, EEF1D, EEF1G, ENG, ESD, GNAI2, GNB1, H1-3, H2BC15, HIP1, KIF11, LAMP1, LAP3, LGALS1, LTBP3, MAPK3, MARCKS, MBTD1, MDH1, MOB1B, MYL12B, MYO1F, MYO3A, NIBAN2, PEBP1, PF4, PGAP1, PLOD1, PPP2RIA, PRSS23, PXDN, RALA, RAP2A, RPS13, RPS3, RPSA, S100A11, SLC44A1, SLC44A2, SLTM, SMG1, SPARC, SRSF8, STRADB, STX11, STXBP2, TGM2, TPP1, TPTE2, TRIM5, TRPM2, TUBA8, TUBB3, VCAN, YWHAE, and ZFN607 at a higher level compared to BM-MSC-EVs, UCB-MSC-EVs, and/or AD-MSC-EVs.

In some embodiments, the HMC-EVs express at least one of the proteins selected from the group consisting of ADIPOQ, CAT, CEP290, IGLV6-57, TAS2R33, and TMEM198 at a lower level compared to BM-MSC-EVs, UCB-MSC-EVs, and/or AD-MSC-EVs.

In some embodiments, the HMC-EVs express at least one of the proteins selected from the group consisting of AKAP9, ALB, ALOX5, APLP2, CD109, CDSN, CHST9, ERC1, F11, ARMCX5, LAMB4, LRRTM2, LTF, MSH6, OAF, OLFML3, PAK6, RGS14, SEMA7A, SURF1, and TRIM4 at a lower level compared to BM-MSC-EVs, UCB-MSC-EVs, and/or AD-MSC-EVs.

In some embodiments, the HMC-EVs of the presently disclosed subject matter may express higher levels of any of the miRNAs listed in Table 21 compared to the HMCs of the presently disclosed subject matter. In some embodiments, the HMC-EVs of the presently disclosed subject matter may express lower levels of any of the miRNAs listed in Table 22 compared to the HMCs of the presently disclosed subject matter.

In an embodiment, genes associated with or involved in the development of neuronal lineage including axon guidance, CREB signaling in neurons, synaptogenesis signaling, or neuroinflammation signaling, are expressed at a higher level in the HMCs of the presently disclosed subject matter compared to AD-MSCs or BM-MSCs.

In another embodiment, the HMCs of the presently disclosed subject matter have a distinct expression profile when compared to mature MSCs, e.g., AD-MSCs or BM-MSCs or UCB-MSCs. Specifically, the HMCs of the presently disclosed subject matter are able to confer neuroprotective effects, and provide neurotrophic factors, i.e., factors involved in supporting neuronal survival, growth, health and recovery. Likewise, the HMC-EVs of the presently disclosed subject matter share a similar profile as the HMCs from which they were derived. Similar signaling pathways enriched in the HMCs are also enriched in the HMC-EVs when compared to other tissue-derived MSCs and EVs.

In an embodiment, the composition comprising HMCs of the presently disclosed subject matter is substantially purified with respect to pluripotent stem cells. In a further embodiment, a composition of HMCs of the presently disclosed subject matter is substantially purified with respect to pluripotent stem cells such that said composition comprises at least about 50%, 55%, 60%, 65%, 70%, 75%, 80%, 85%, 90%, 91%, 92%, 93%, 94%, 95%, 96%, 97%, 98%, 99%, or 100% HMCs. The pluripotent stem cells may be any pluripotent stem cells described herein.

The composition may comprise less than about 50%, 45%, 40%, 35%, 30%, 25%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% pluripotent stem cells. The composition may be devoid of pluripotent stem cells.

In some embodiments, the composition comprising HMC-EVs of the presently disclosed subject matter is substantially purified with respect to the HMCs. In a further embodiment, a composition of HMC-EVs of the presently disclosed subject matter is substantially purified with respect to HMCs such that said composition comprises at least about 50%, 55%, 60%, 65%, 70%, 75%, 80%, 85%, 90%, 91%, 92%, 93%, 94%, 95%, 96%, 97%, 98%, 99%, or 100% HMC-EVs.

The composition may comprise less than about 50%, 45%, 40%, 35%, 30%, 25%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% HMCs.

In another embodiment of the instant presently disclosed subject matter, a composition of HMCs and/or HMC-EVs generated by any one or more of the processes of the instant presently disclosed subject matter does not form a teratoma when introduced into a host.

In an exemplary aspect, the present disclosure provides a composition comprising at least 10⁴, 10⁵, 10⁶, 10⁷, 10⁸ or 10⁹ HMCs. In a specific embodiment, the composition comprises 10⁶ HMCs and less than one percent of any other cell type, wherein the mesenchymal stem cells have replicative capacity to undergo at least 10 population doublings in cell culture with less than 25 percent of the cells undergoing cell death, senescing or differentiating into non-HMC cells by the tenth population doubling.

The HMCs may have replicative rates to undergo at least 10 population doublings in cell culture in less than 25 days. The HMCs may have a mean terminal restriction fragment length (TRF) that may be longer than 8 kb. The HMCs may have a statistically significant decreased content and/or enzymatic activity, relative to mesenchymal stem cell preparations derived from bone marrow that have undergone five population doublings, of proteins involved in one or more of (i) cell cycle regulation and cellular aging, (ii) cellular energy and/or lipid metabolism, and (iii) apoptosis. The HMCs may have a statistically significant increased content and/or enzymatic activity of proteins involved in cytoskeleton structure and cellular dynamics relating thereto, relative to mesenchymal stem cell preparations derived from bone marrow. The HMCs may not undergo more than a 75 percent increase in cells having a forward-scattered light value, measured by flow cytometry, greater than 5,000,000 over 10 population doublings in culture.

In an embodiment of the instant presently disclosed subject matter, a preparation of the subject HMCs (e.g., generated by culturing hemangioblasts) is provided, wherein said preparation comprises substantially similar levels of p53 and p21 protein, or wherein the levels of p53 as compared to p21 are 1.5, 2, 3, 4, 5, 6, 7, 8, 9, or 10 times greater. In an embodiment of the instant presently disclosed subject matter, a pharmaceutical preparation of the subject HMCs (e.g., generated by culturing hemangioblasts) is provided, wherein said pharmaceutical preparation comprises substantially similar levels of p53 and p21 protein, or wherein the levels of p53 as compared to p21 are 1.5, 2, 3, 4, 5, 6, 7, 8, 9, or 10 times greater.

In an embodiment, the presently disclosed subject matter provides a composition comprising HMCs, wherein the comprises a substantially similar percentage of HMCs positive for p53 and p21 protein, or wherein the percentage of HMCs positive for p53 as compared to p21 are 1.5, 2, 3, 4, 5, 6, 7, 8, 9, or 10 times greater.

In one embodiment, the present disclosure provides a composition comprising at least about 10³ to about 10¹³ HMC-EVs. In another embodiment, the present disclosure provides a composition comprising at least 10³, 10⁴, 10⁵, 10⁶, 10⁷, 10⁸, 10⁹, 10⁷, 10⁸, 10⁹, 10¹⁰, 10¹¹, 10¹², or 10¹³ HMC-EVs.

Methods of Determining Neurite Outgrowth of HMC and/or HMC-EV Populations.

The presently disclosed subject matter also provides a method of determining effects of the HMC and/or HMC-EVs on neurons, such as neurite outgrowth. In an aspect, the presently disclosed subject matter provides a method of determining neurite outgrowth of an HMC and/or HMC-EV population. In an embodiment, the method comprises (a) preparing a mixed neuronal culture from an isolated cerebral cortex, (b) plating the HMC and/or HMC-EV population on a permeable membrane, (c) applying strain on the mixed neuronal culture, (d) overlaying the strained mixed neuronal culture with the permeable membrane of step (b), and (e) measuring neurite outgrowth of the mixed neuronal culture. In an embodiment, the method further comprises determining gene expression of the mixed neuronal culture in the presence and absence of the HMC and/or HMC-EV population. In another embodiment, the strain is a physical scratch made in the mixed neuronal culture. In another embodiment, the strain is vacuum pressure and positive air pressure applied to the mixed neuronal culture. In yet another embodiment, the strain may be applied at 15% to 0% stretching oscillations. In an embodiment, the stretching oscillations may be applied at 15%, 12.5%, 10%, 7.5%, 5%, 2.5%, or 0% cycles.

### Pharmaceutical Preparations Comprising HMCs and HMC-EVs

Pharmaceutical preparations of the instant presently disclosed subject matter may comprise any of the HMCs or compositions of HMCs described herein, and/or HMC-EVs. Pharmaceutical preparations comprising HMCs and/or HMC-EVs of the presently disclosed subject matter may be formulated with a pharmaceutically acceptable carrier. For example, HMCs and/or HMC-EVs of the presently disclosed subject matter may be administered alone or as a component of a pharmaceutical formulation, wherein said HMCs and/or HMC-EVs may be formulated for administration in any convenient way for use in medicine. One embodiment provides a pharmaceutical preparation of HMCs and/or HMC-EVs comprising said HMCs and/or HMC-EVs in combination with one or more pharmaceutically acceptable sterile isotonic aqueous or non-aqueous solutions selected from the group consisting of: dispersions, suspensions, emulsions, sterile powders optionally reconstituted into sterile injectable solutions or dispersions just prior to use, antioxidants, buffers, bactericides, solutes or suspending and thickening agents.

Exemplary pharmaceutical preparations of the present disclosure may be any formulation suitable for use in treating a human patient, such as pyrogen-free or essentially pyrogen-free, and pathogen-free.

The preparation comprising HMCs and/or HMC-EVs used in the methods described herein may be transplanted in a suspension, gel, colloid, slurry, or mixture. Also, at the time of injection, cryopreserved HMCs and/or HMC-EVs may be resuspended with commercially available balanced salt solution to achieve the desired osmolality and concentration for administration by injection (i.e., bolus or intravenous).

One aspect of the presently disclosed subject matter relates to a pharmaceutical preparation suitable for use in a mammalian patient, comprising at least 10⁴, 10⁵, 10⁶, 10⁷, 10⁸, 10⁹, 10¹⁰, 10¹¹, 10¹², or 10¹³ HMCs and/or HMC-EVs and a pharmaceutically acceptable carrier. Yet another aspect of the presently disclosed subject matter provides a cryogenic cell bank comprising at least 10⁸, 10⁹, 10¹⁰, 10¹¹, 10¹² or even 10¹³ HMCs and/or HMC-EVs. Still another aspect of the presently disclosed subject matter provides a pharmaceutical preparation free of or substantially free of non-human cells and/or non-human animal products, comprising at least 10⁴, 10⁵, 10⁶, 10⁷, 10⁸ 10⁹, 10¹⁰, 10¹¹, 10¹², or 10¹³ HMCs and/or HMC-EVs and less than 1% of any other cell type, more preferably less than 0.1%, 0.01% or even 0.001% of any other cell type.

Concentrations for administration of pharmaceutical preparations of HMCs and/or HMC-EVs may be at any amount that is effective and, for example, substantially free of PSCs. For example, the pharmaceutical preparations may comprise the numbers and types of HMCs and/or HMC-EVs described herein. In a particular embodiment, the pharmaceutical preparations of HMCs and/or HMC-EVs comprise about 1×10⁶ to about 1×10⁷, about 1×10⁷ to about 1×10⁸, about 1×10⁸ to about 1×10⁹, about 1×10⁹ to about 1×10¹⁰, about 1×10¹⁰ to about 1×10¹¹, about 1×10¹¹ to about 1×10¹², or about 1×10¹² to about 1×10¹³ of the HMCs and/or HMC-EVs for systemic administration to a host in need thereof or about 1×10⁴ to about 1×10³, about 1×10³ to about 1×10⁶, 1×10⁶ to about 1×10⁷, about 1×10⁷ to about 1×10⁸, about 1×10⁸ to about 1×10⁹, about 1×10⁹ to about 1×10¹⁰, about 1×10¹⁰ to about 1×10¹¹, about 1×10¹¹ to about 1×10¹², or about 1×10¹² to about 1×10¹³ of said HMCs and/or HMC-EVs for local administration to a host in need thereof.

### Methods of Treating Brain Injury

The HMCs and/or HMC-EVs and pharmaceutical preparations comprising HMCs and/or HMC-EVs described herein may be used for treating brain injury, e.g., stroke, or optic neuropathy. In particular, the instant presently disclosed subject matter provides methods for treating or preventing brain injuries described herein comprising administering an effective amount of HMCs and/or HMC-EVs, wherein the HMCs are obtained by in vitro differentiation of pluripotent stem cells. In another embodiment, the HMCs are obtained by in vitro differentiation of hemangioblasts.

In an embodiment, brain injury is selected from traumatic brain injury, acquired brain injury, anoxic brain injury, diffuse axonal brain injury, focal brain injury, subdural hematoma, brain aneurysm, coma, stroke, optic neuropathy, and cerebral palsy. In a particular embodiment, the brain injury is traumatic brain injury. In another embodiment, the brain injury is cerebral palsy. In yet another embodiment, the brain injury is stroke. In another embodiment, the brain injury is optic neuropathy.

The HMCs and/or HMC-EVs of the instant presently disclosed subject matter may be administered systemically or locally. The HMCs and/or HMC-EVs may be administered using modalities known in the art including, but not limited to, injection via intravenous, intracranial, intrathecal, intracerebral, intracisternal, intramuscular, intraperitoneal, intravitreal, or other routes of administration, or local implantation, dependent on the particular pathology being treated.

The HMCs and/or HMC-EVs of the instant presently disclosed subject matter may be administered via local implantation, such as intracranial implantation, wherein a delivery device is utilized. Delivery devices of the instant presently disclosed subject matter are biocompatible and biodegradable. A delivery device of the instant presently disclosed subject matter can be manufactured using materials selected from the group comprising biocompatible fibers, biocompatible yarns, biocompatible foams, aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biopolymers; homopolymers and copolymers of lactide, glycolide, epsilon-caprolactone, para-dioxanone, trimethylene carbonate; homopolymers and copolymers of lactide, glycolide, epsilon-caprolactone, para-dioxanone, trimethylene carbonate, fibrillar collagen, non-fibrillar collagen, collagens not treated with pepsin, collagens combined with other polymers, growth factors, extracellular matrix proteins, biologically relevant peptide fragments, hepatocyte growth factor, platelet-derived growth factors, platelet rich plasma, insulin growth factor, growth differentiation factor, vascular endothelial cell-derived growth factor, nicotinamide, glucagon like peptides, tenascin-C, laminin, anti-rejection agents, analgesics, anti-oxidants, anti-apoptotic agents anti-inflammatory agents and cytostatic agents. In some embodiments, the HMCs and/or HMC-EVs are delivered through a slow release device, e.g., transdermal microneedle patch.

The particular treatment regimen, route of administration, and adjuvant therapy may be tailored based on the particular pathology, the severity of the pathology, and the patient's overall health. Administration of the HMCs and/or HMC-EVs may be effective to reduce the severity of the manifestations of a pathology or and/or to prevent further degeneration of the manifestation of a pathology.

In some embodiments, administration of the HMCs results in preservation of myelin. In some embodiments, administration of the HMCs results in suppression of neuroinflammatory response in a subject. In some embodiments, administration of the HMCs results in reduction of microglial and astrocyte activation in the brain. In some embodiments, administration of the HMCs results in stimulation and/or activation of pathways involved in cell survival. In some embodiments, administration of the HMCs results in stimulation of expression of a neuroprotective gene in the brain. In some embodiments, the neuroprotective gene is selected from the group consisting of heat shock protein family B member 1 (HSPB1), insulin-like growth factor 1 (IGF2), and secreted phosphoprotein 1 (SPP1). In some embodiments, administration of the HMCs results in stimulation and/or activation of pathways involved in synaptic transmission in the brain. In some embodiments, administration of the HMCs results in reduction of apoptosis. In some embodiments, administration of the HMCs results in stimulation and/or activation of pathways involved in development of neuronal lineage, e.g., axon guidance, BREB signaling in neurons, or synaptogenesis signaling.

In some embodiments, administration of HMC-EVs results in an increase in the oligodendrocyte and precursor cells in the brain. In some embodiments, administration of HMC-EVs results in preservation of myelin in the brain. In some embodiments, administration of HMC-EVs results in suppression of neuroinflammatory response in the subject. In some embodiments, administration of HMC-EVs results in reduction of microglial and astrocyte activation in the brain. In some embodiments, administration of HMC-EVs results in prevention or reduction of oxidative damage in neurons. In some embodiments, administration of extracellular HMC-EVs results in prevention or reduction of neuronal death due to glutamate excitotoxicity injury.

A treatment modality of the presently disclosed subject matter may comprise the administration of a single dose of HMCs and/or HMC-EVs. Alternatively, treatment modalities described herein may comprise a course of therapy where HMCs and/or HMC-EVs are administered multiple times over some period of time. Exemplary courses of treatment may comprise weekly, biweekly, monthly, quarterly, biannually, or yearly treatments. Alternatively, treatment may proceed in phases whereby multiple doses are required initially (e.g., daily doses for the first week), and subsequently fewer and less frequent doses are needed.

The HMCs and/or HMC-EVs may be administered separately or in combination. In some embodiments, the methods comprise administering to the subject an effective amount of HMCs. In other embodiments, the methods comprise administering to the subject an effective amount of HMC-EVs. In another embodiment, the methods comprise administering to the subject an effective amount of HMCs and an effective amount of HMC-EVs.

The HMCs and HMC-EVs can be administered simultaneously or sequentially. In one embodiment, the HMCs and the HMC-EVs are mixed together before administering to the subject. In another embodiments, the subject receives an effective amount of HMCs, followed by an effective amount of HMC-EVs. Alternatively, the subject receives an effective amount of HMC-EVs, followed by an effective amount of HMCs.

In one embodiment, the HMCs and/or HMC-EVs are administered to a patient one or more times periodically throughout the life of a patient. In a further embodiment of the instant presently disclosed subject matter, the HMCs and/or HMC-EVs are administered once per year, once every 6-12 months, once every 3-6 months, once every 1-3 months, or once every 1-4 weeks. Alternatively, more frequent administration may be desirable for certain conditions or disorders. In an embodiment of the instant presently disclosed subject matter, the HMCs and/or HMC-EVs are administered via a device once, more than once, periodically throughout the lifetime of the patient, or as necessary for the particular patient and patient's pathology being treated. Similarly contemplated is a therapeutic regimen that changes over time. For example, more frequent treatment may be needed at the outset (e.g., daily or weekly treatment). Over time, as the patient's condition improves, less frequent treatment or even no further treatment may be needed.

In some embodiments, about 20 million, about 40 million, about 60 million, about 80 million, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, about 500 million, about 520 million, about 540 million, about 560 million, about 580 million, about 600 million, about 620 million, about 640 million, about 660 million, about 680 million, about 700 million, about 720 million, about 740 million, about 760 million, about 780 million, about 800 million, about 820 million, about 840 million, about 860 million, about 880 million, about 900 million, about 920 million, about 940 million, about 960 million, or about 980 million MSCs and/or MSC-EVs are administered into the subject. In some embodiments, about 1 billion, about 2 billion, about 3 billion, about 4 billion or about 5 billion HMCs and/or HMC-EVs or more are administered. In some embodiments, the number of HMCs and/or HMC-EVs ranges from between about 20 million to about 4 billion, between about 40 million to about 1 billion, between about 60 million to about 750 million, between about 80 million to about 400 million, between about 100 million to about 350 million, and between about 175 million to about 250 million.

The methods described herein may further comprise the step of monitoring the efficacy of treatment or prevention using methods known in the art.

### EXAMPLES

The following examples are not intended to limit the presently disclosed subject matter in any way.

### Example 1—Generating HMCs from Hemangioblasts

Hemangioblasts were generated from single-blastomere derived human ESC line, MA09 (Klimanskaya et al., Nature 444 (2006) 481-485). First, a 10 cm plate was coated with 0.1% gelatin and irradiated MEF was added at a concentration of about 25,000 cells/cm² in MEF media (high glucose DMEM+10% FCS) the day before adding ESCs to the plate. The MEF media was then aspirated, rinsed with PBS, and replaced with Reprocell Primate media (Reprocell) plus 10 ng/mL bFGF. A split of MA09 cells were added to the dish and fed with fresh media daily. The MA09s were cultured in Reprocell Primate Media plus 10 ng/mL bFGF until about 90% confluent. The MA09s were then harvested with 0.05% trypsin/EDTA or Reprocell dissociation buffer (Reprocell). After the cells detached, the cells were rinsed and collected. The cells were spun down at 300×g for 10 min. The supernatant was aspirated and the cell pellet was resuspended in Stemline II (Sigma) (plus pen/strep and L-glutamine) plus 50 ng/mL VEGF and 50 ng/mL BMP4. The MA09 ESCs were plated in 2×10 cm ultra low adherence plate (Corning) in 15 ml Stemline II medium (Sigma) supplemented with 50 ng/ml of VEGF and 50 ng/ml of BMP-4 (R & D or Peprotech) and incubated at 37° C. with 5% CO₂. After 40-48 hours, half of the medium (1.5 ml) was replaced with fresh Stemline II medium supplemented with 50 ng/ml of VEGF, 50 ng/ml of BMP-4, and 40-45 ng/ml bFGF so that the final concentration of bFGF ends up being 20-22.5 ng/ml bFGF, and continued incubation for an additional 40-48 hours (i.e., 3.5-4 days total).

Clusters of cells (embryoid bodies; EBs) were dissociated and plated as single cells in serum-free semisolid blast-colony growth medium (BGM). Specifically, clusters of cells were dissociated with trypsin for 2-5 min. or until clumps start to break up. The cell suspension was pipetted up and down and then DMEM+10% FCS was added to inactivate the trypsin. Cells were then passed through a 40 μm or 70 μm strainer to obtain a single cell suspension. Cells were then counted and resuspended in Stemline II medium at 1-1.5×10⁶ cells/ml.

The single cell suspension was mixed with hemangioblast (HB) Growth Medium (H4536 based medium recipe: base medium methylcellulose product H4536 (StemCell Technologies) plus penicillin/streptomycin (pen/strp), Excyte growth supplement (Millipore), and the cytokines, Flt3-ligand (FL) at 50 ng/ml, vascular endothelial growth factor (VEGF) at 50 ng/ml, thrombopoietin (TPO) at 50 ng/ml, and basic fibroblast growth factor (bFGF) at 20-30 ng/ml) for a final concentration of about 1×10⁵ cells/ml with a brief vortex, and allowing the bubbles to settle. The cell mixture was then transferred to 4×10 cm ultra low adherence plates by using a syringe (30 ml) attached with an 18G needle, and incubated at 37° C. with 5% CO₂ for 8-12 days. HBs will begin to appear within 3 or 4 days and continue to populate the plates and may be harvested between days 7-12 of culture. The HBs were harvested on day 9 of culture and frozen down.

The frozen HBs were thawed and replated onto Matrigel-coated tissue culture plates in MSC medium [α-MEM without nucleosides (Hyclone), 20% Defined FBS—Heat Inactivated (Hyclone), 1× Glutamax (Gibco), 1×MEM non-essential amino acids (Gibco), and 1× penicillin/streptomycin]. The cells were cultured for about 4-5 days and then passaged, and repeated for up to three passages (P3) to generate HMCs. The P3 HMCs (“MARP12” cells) were frozen down for further use.

### Example 2—Traumatic Brain Injury (TBD In Vivo Study

The HMCs obtained according to Example 1 were thawed and cultured in MSC medium described above for about 4 days in 37° C., 5% CO₂ in T225 culture flasks at about 4500 cells/cm². To harvest the cells for administration, the cells were washed with PBS, dissociated from the flasks with trypsin, and the trypsin was inactivated with addition of MSC medium. The cells were collected in 50 ml conical tubes and centrifuged at 300×g for 10 min. The supernatant was aspirated and 1 ml of GS2 buffer [for 552.2 mL of GS2: 0.9% Sodium Chloride Irrigation USP (408.6 mL); 5% Dextrose/0.9% Sodium Chloride, Injection USP (33.2 mL), and BSS Irrigation Solution (110.4 mL)], which is described in WO 2017/031312 and is incorporated herein by reference in its entirety, was added to each tube. The cells were strained through a 100 μm cell strainer and centrifuged at 300×g for 5 min. The supernatant was aspirated and resuspended in GS2. The cells obtained are passage 4 (P4) HMCs.

Mild-to moderate experimental traumatic brain injury (TBI) was induced in 56 Sprague Dawley Rats by controlled cortical impact (CCI) (Lee et al., Theranostics 9:1029-1046 (2019)). Cells were injected locally by intracerebral (IC) transplantation or systemically (iv) into the rats and sacrificed at early or late time points according to Table 1.

TABLE 1

| Groups | Animals | Time-points | End-points |
|---|---|---|---|
| EARLY | | | |
| IC Local | 7 | Treatment with cells | Cortical and |
| Administration | | or vehicle 7 days | Hippocampal cell |
| Vehicle (3 ul-10 ul | | post CCI. | loss- H&E |
| GS2) | | Animals sacrificed 7 | staining and CA3 |
| IC Local | 7 | days post treatment | neuron counting |
| Administration MSCs | | (14 days post CCI). | Microgliosis- |
| (400,000 cells in 3 ul- | | | DCX, OX6. |
| 10 ul GS2) | | | IBA-1 staining |
| I.V. (jugular vein) | 7 | | IHC for human |
| Admin Vehicle (500 ul | | | cells |
| GS2) | | | Swing Test |
| I.V. (jugular vein) | 7 | | Bederson Test |
| Admin MSCs (4 × 10⁶ | | | |
| cells in 500 ul GS2) | | | |
| LATE | | | |
| IC Local | 7 | Treatment with cells | All end points as |
| Administration | | or vehicle 7 days | Early groups |
| Vehicle (3 ul-10 ul) | | post CCI. | |
| IC Local | 7 | Behavioral testing | |
| Administration | | every 7 days from | |
| MSCs (400,000 cells | | Day 0 (CCI) to Day | |
| in 3 ul-10 ul) | | 56 plus baseline. | |
| I.V. (jugular vein) | 7 | Animals sacrificed | |
| Admin Vehicle (500 ul | | at Day 56. | |
| GS2) | | | |
| I.V. (jugular vein) | 7 | | |
| Admin MSCs (4 × 10⁶ | | | |
| cells in 500 ul GS2) | | | |

The rats were studied according to the following schedule:

### Early

Day −1: Swing test and Bederson test for baseline
Day 0: Controlled Cortical Impact performed on all groups
Day 7: All groups treated with cells or vehicle, locally or intravenously; Swing test and Bederson test post treatment for all groups
Day 14: Swing and Bederson Tests for all groups; All groups sacrificed; H&E staining, CA3 neuron counting, DCX, OX6, IBA-1 staining, IHC for human cells on all groups

### Late

Day −1: Swing Test and Bederson Test for baseline for all groups
Day 0: Controlled Cortical Impact performed on all groups
Day 7: All groups treated with cells or vehicle, locally or intravenously; Swing and Bederson tests post treatment for all groups
Day 14: Swing and Bederson tests for all groups
Day 28: Swing and Bederson tests for all groups
Day 35: Swing and Bederson tests for all groups
Day 42: Swing and Bederson tests for all groups
Day 49: Swing and Bederson tests for all groups
Day 56: Swing and Bederson tests for all groups; All groups sacrificed; H&E staining, CA3 neuron counting, DCX, OX6, IBA-1 staining, IHC for human cells on all groups.
   Results from Behavioral Tests

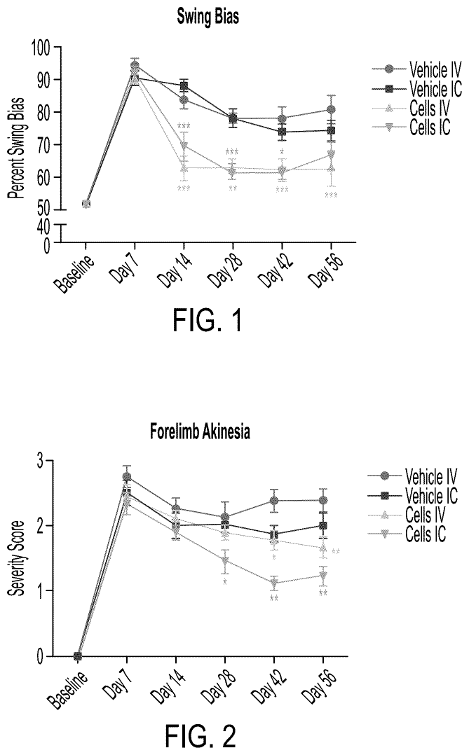

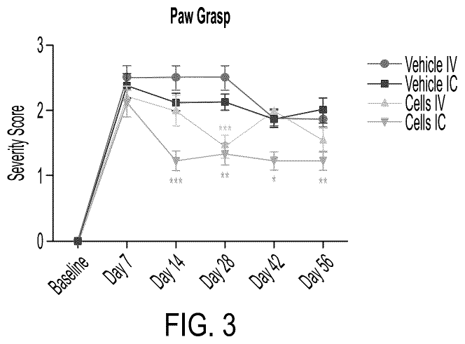

The CCI in vivo TBI model causes significant behavioral deficits of the rats up to 56 days post-injury. Intracerebral (IC) transplantation of the HMCs significantly rescued against behavior deficits compared to their respective vehicles, including elevated body swing test (EBST) from day 14 to 42 after transplantation (FIG. 1), forelimb akinesia starting at day 28 up to day 56 after transplantation (FIG. 2), and paw grasp from day 14 to day 56 after transplantation (FIG. 3). Intravenous (IV) transplantation of the HMCs also significantly rescued against behavior deficits compared to their respective vehicles, including EBST from day 14 up to day 56 after transplantation (FIG. 1), forelimb akinesia starting at day 42 to day 56 after transplantation (FIG. 2), and paw grasp at day 28 after transplantation (FIG. 3). These findings support the use of HMCs for treatment of TBI.

Results from Histology

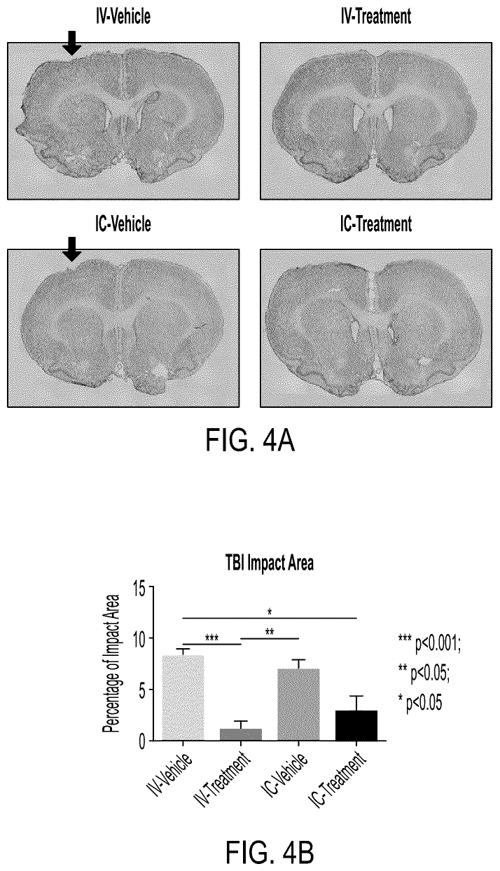

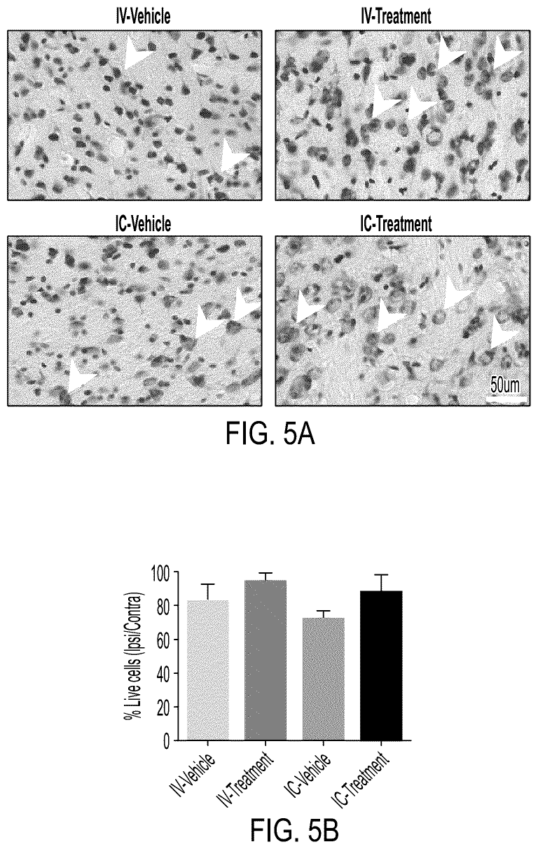

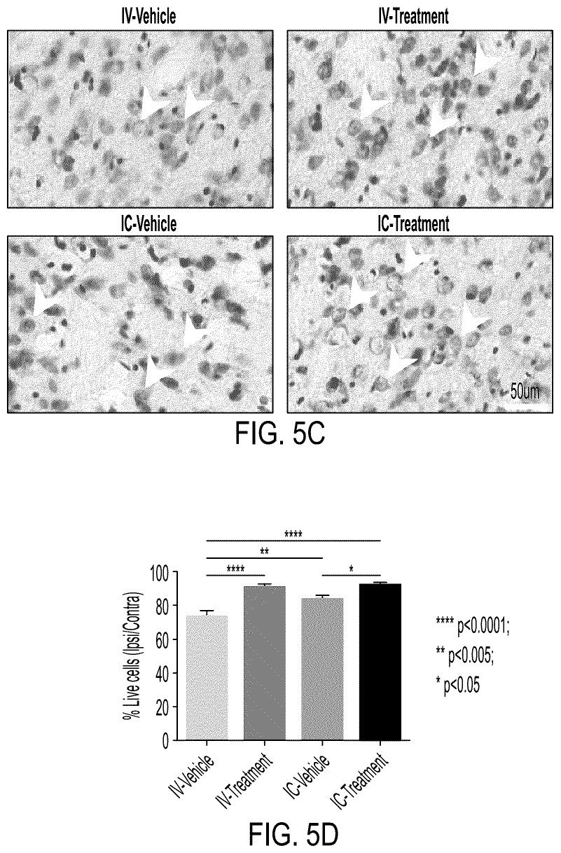

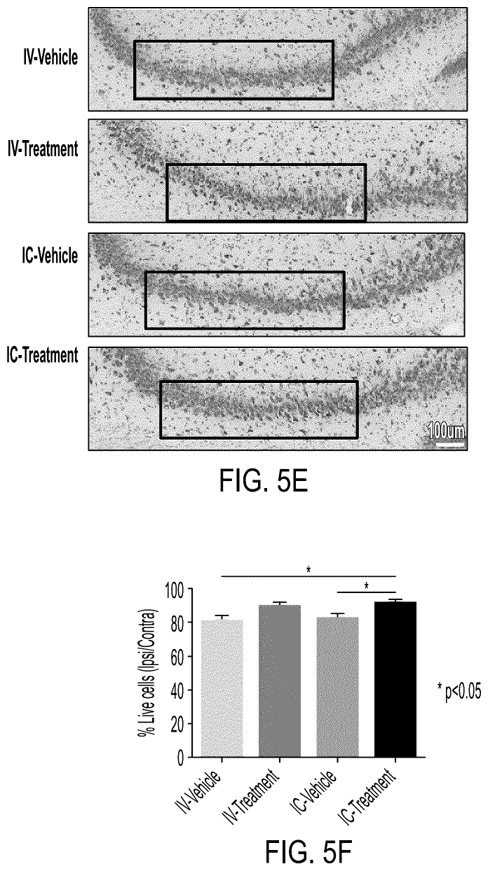

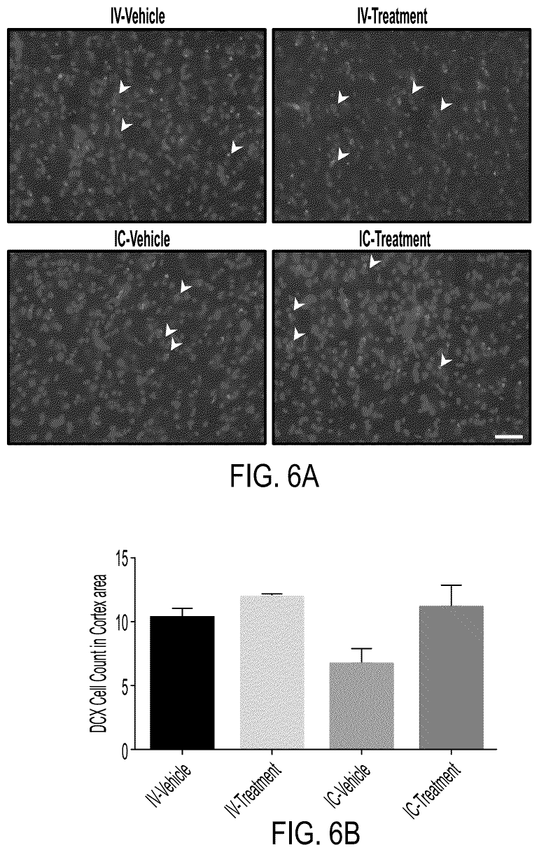

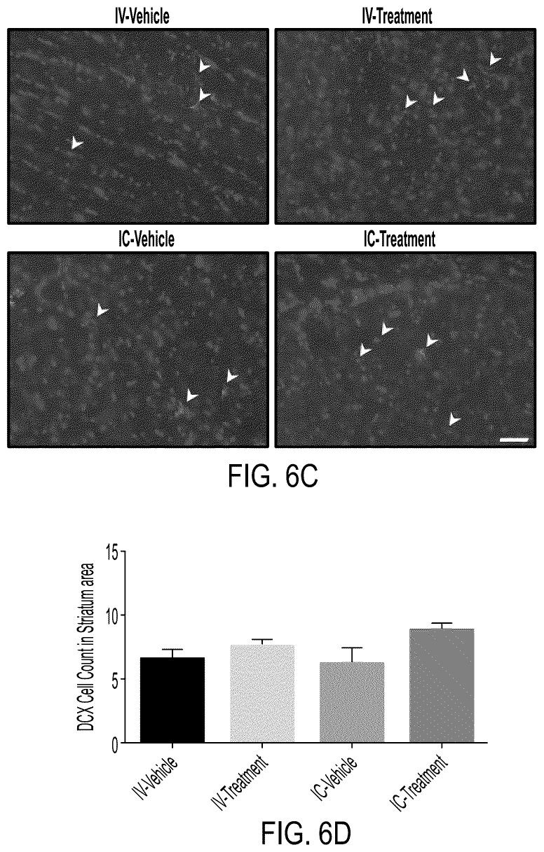

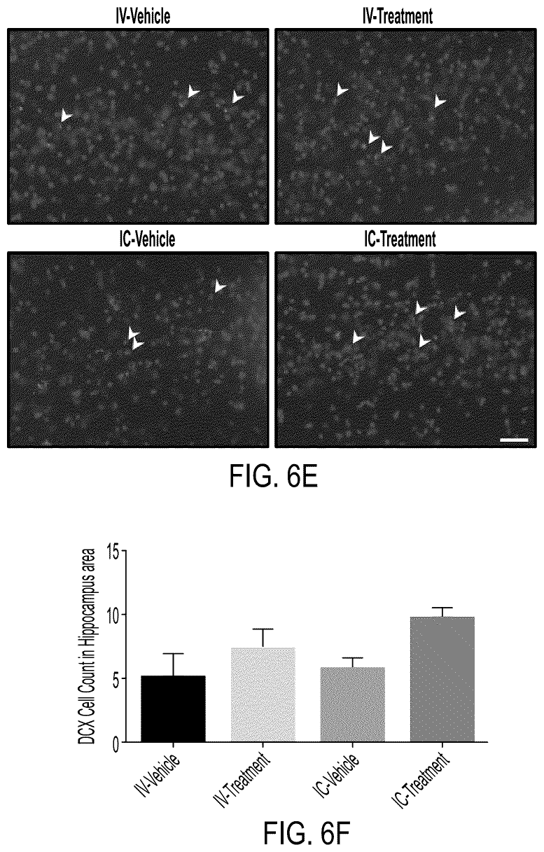

The CCI in vivo model causes significant histopathological effects in the rats post-injury. IV and IC transplantation of the HMCs demonstrated neuroprotective effects compared to their respective vehicles. For example, H&E staining showed a reduction in tissue loss compared to vehicle (FIGS. 4A-B), Nissl staining demonstrated a neuroprotective effect of HMC administration by reducing cell death (FIGS. 5A-F), and doublecortin (DCX) staining showed a slight increase in neurogenesis following the administration of HMCs post-injury (FIGS. 6A-F).

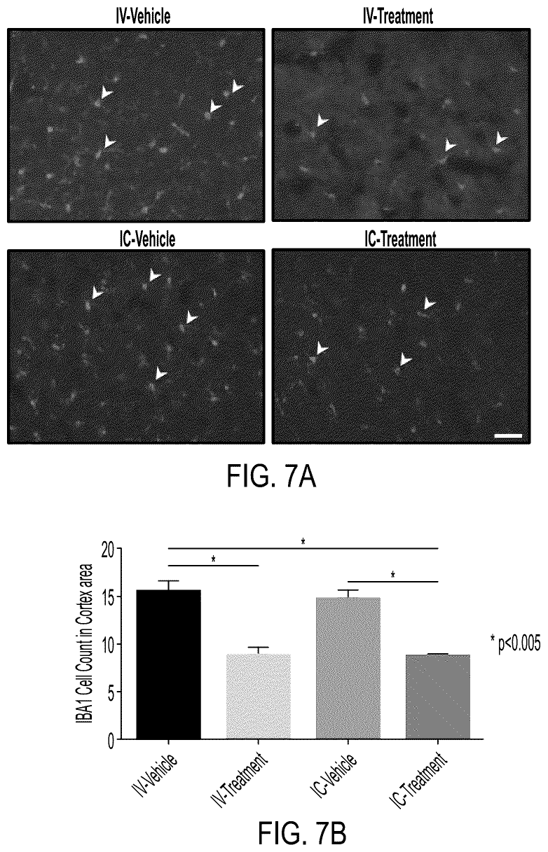

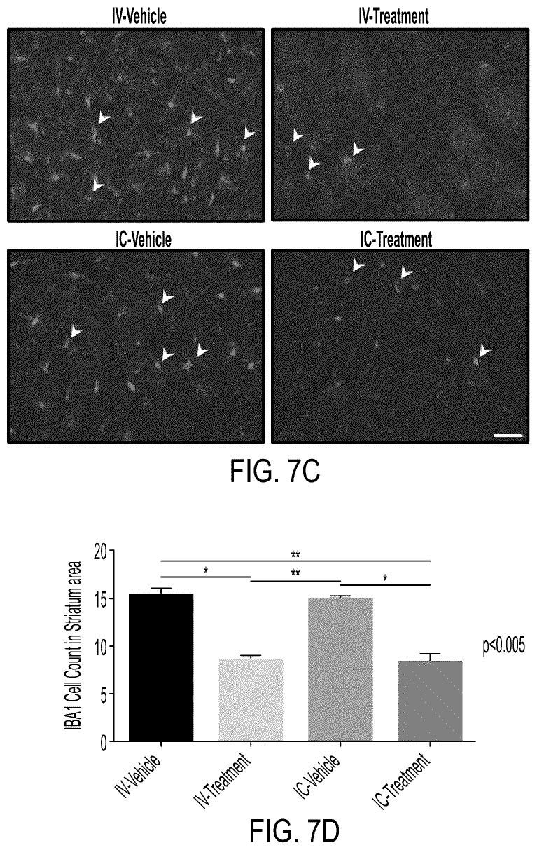

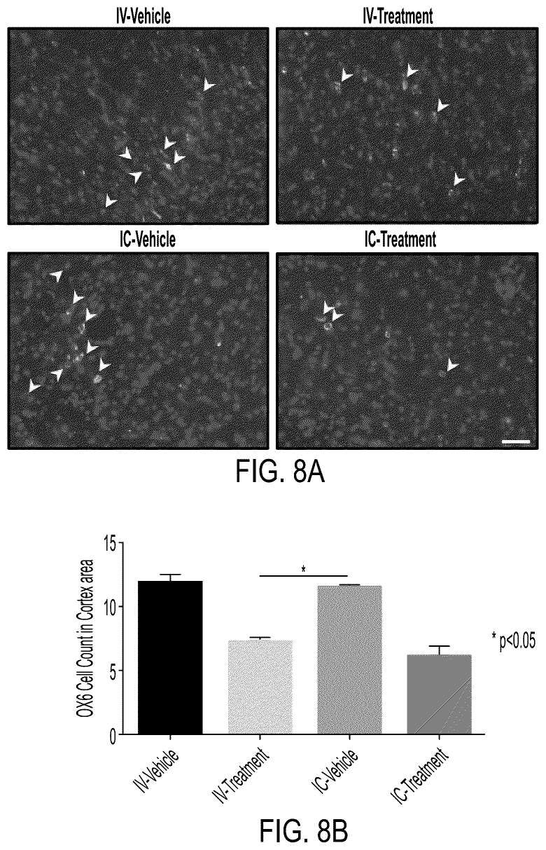

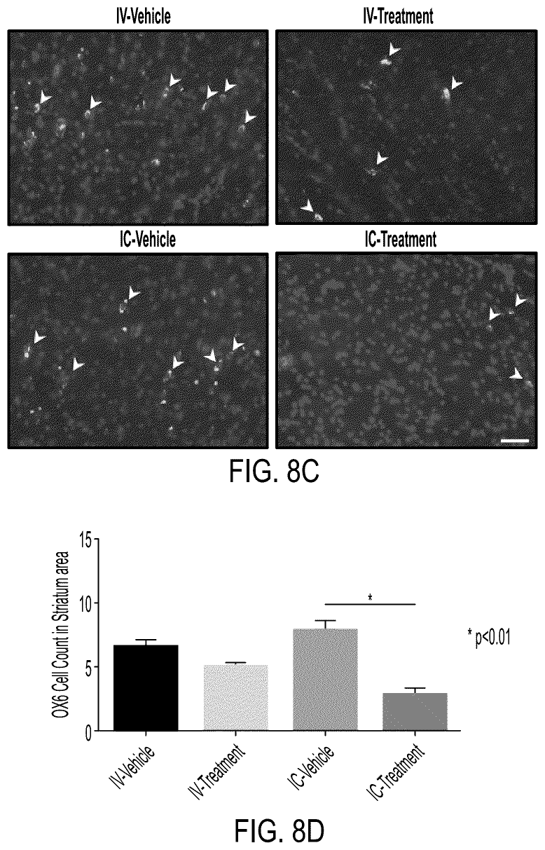

IV and IC transplantation of the HMCs also significantly reduced the activation of microglia and macrophages compared to their respective vehicles. Iba1 (FIGS. 7A-D) and OX6 (FIGS. 8A-D) staining demonstrated that the HMCs reduced the presence of microglia and macrophages, respectively, in the cortex and striatum post-injury.

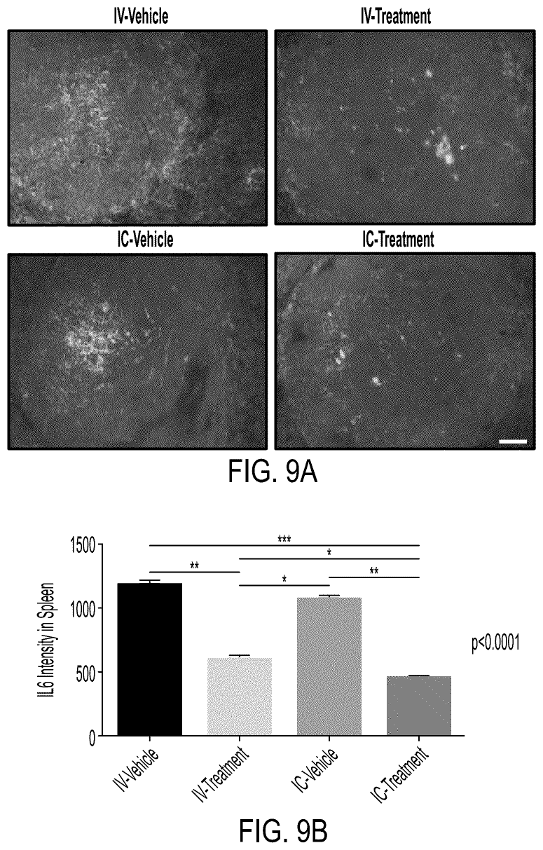

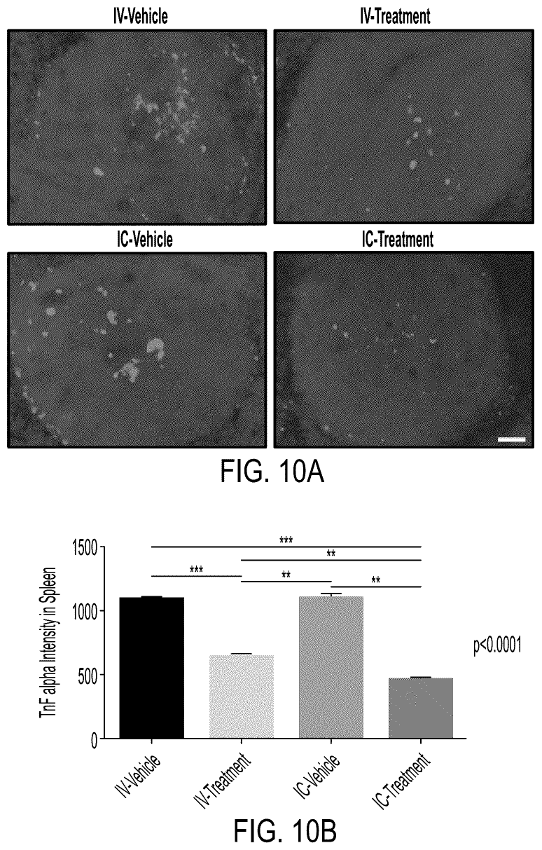

Further, IV and IC transplantation of the HMCs significantly reduced inflammatory markers in the spleen compared to their respective vehicles. A reduction in I16 (FIGS. 9A-B) and TNF-alpha (FIGS. 10A-B) staining in the spleen demonstrates the HMCs reduced inflammation post-injury.

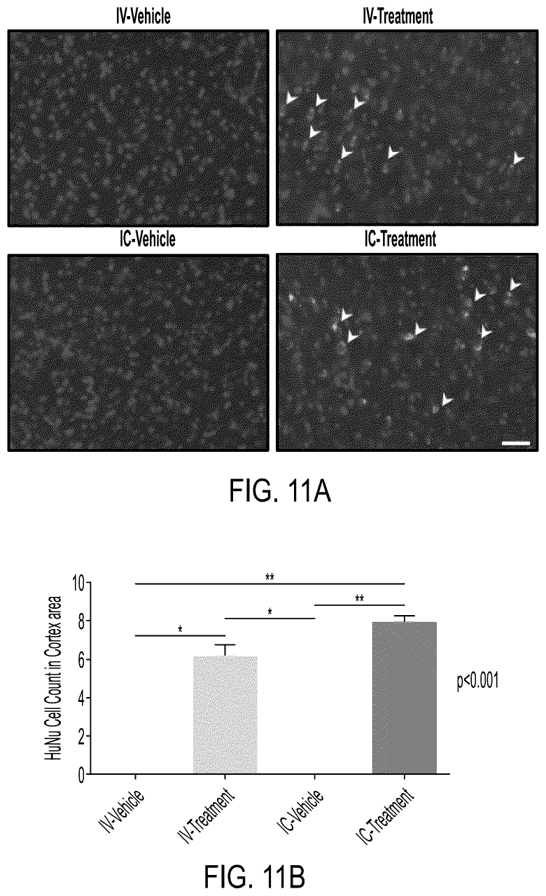

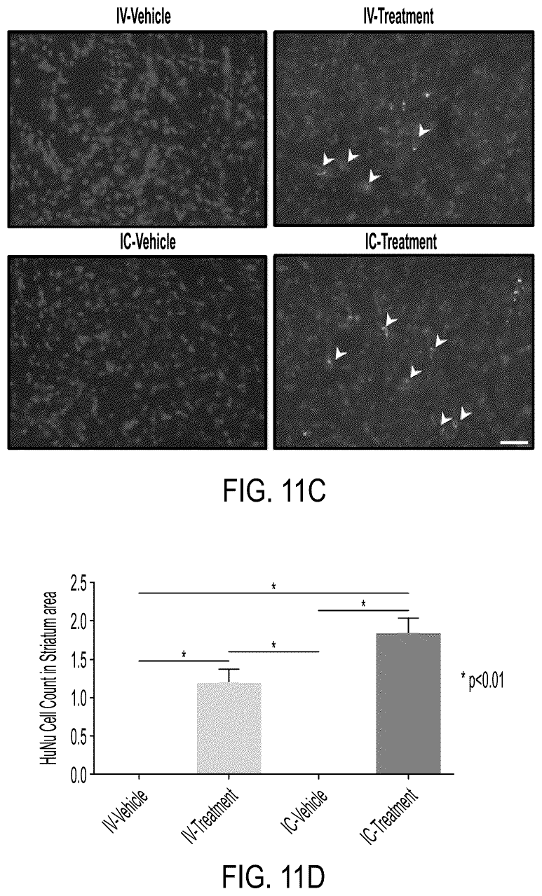

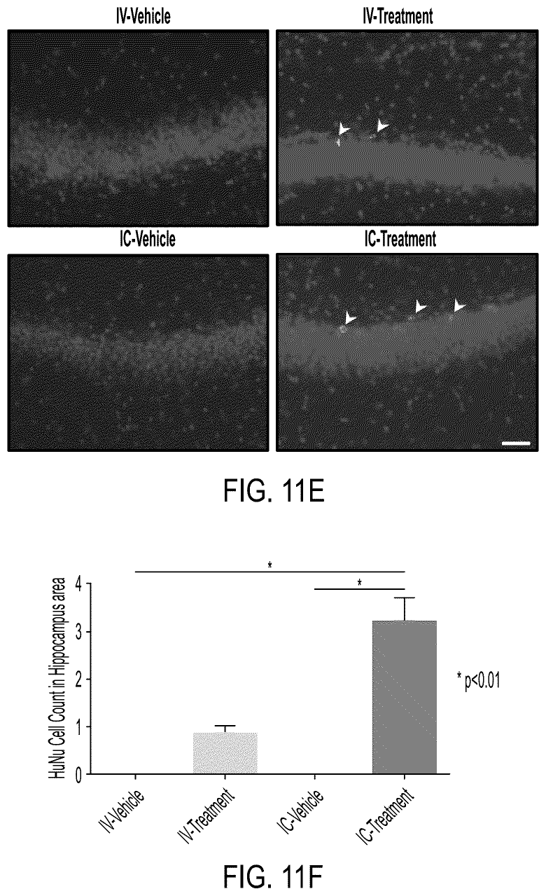

IV and IC transplantation of the HMCs also resulted in migration of HMCs across the blood brain barrier (BBB) to the cortex, striatum, and hippocampus as shown by HuNu staining (FIGS. 11A-F).

These finding support the use of HMCs for treatment of TBI.

### Example 3—In Vitro Migration Assay of HMCs

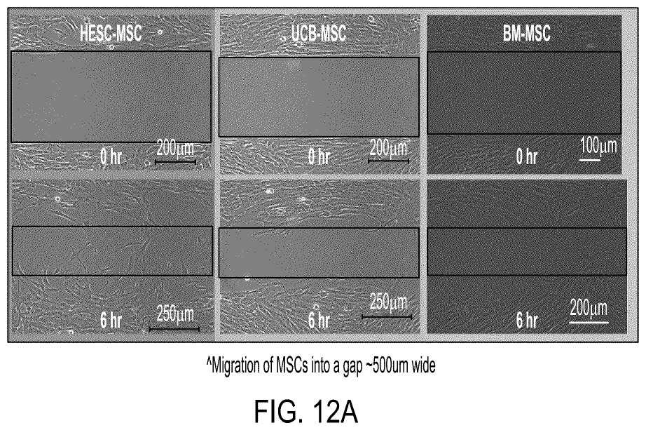

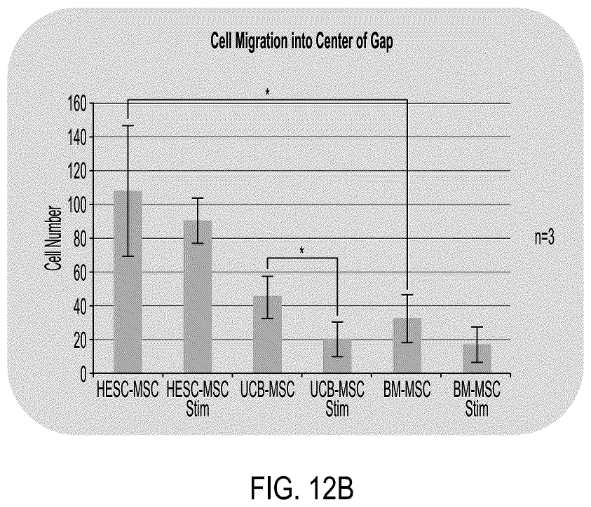

HMCs were generated from the same bank of frozen hemangioblasts described in Example 1. Three separate lots of HMCs were generated, frozen at P4, thawed and cultured for 4 days, and the passage 5 (P5) cells were harvested according to the method described in Example 1. MSCs isolated from bone marrow (BM-MSCs) and umbilical cord blood (UCB-MSCs) were used as controls. Each of the HMCs, BM-MSCs, and UCB-MSCs were seeded into two wells of an ibidi insert with a defined gap in between and allowed to adhere overnight. Inserts were removed, leaving a 500 μm gap. Cells were washed and MSC media (described in Example 1) was added to the chamber, with or without stimulation with 25 ng/mL TNF-α+50 ng/mL IFN-γ. Cells were incubated for 6 hours at 37° C. Pictures were then taken of the non-stimulated cells (FIG. 12A) and cells that had migrated into the center of the gap (middle ˜250 μm) were counted visually (FIG. 12B), using ImageJ, an open source image processing program (Schneider et al., Nature Methods 9:671-675 (2012)). As can be seen from FIGS. 12A-B, the HMCs (hESC-MSCs) had a greater capacity for cell migration than BM-MSCs or UCB-MSCs.

### Example 4—In Vitro Neurite Outgrowth/Neuron Migration in the Presence of HMCs

Rat primary mixed neuronal cultures were prepared from whole brains of E18 Sprague Dawley rat pups obtained from BrainBits, LLC (Springfield, IL). The midbrain, cerebellum, and hippocampus were removed to isolate the cerebral cortex. Cells were dissociated from the tissue and cultured for 14 days to allow for maturation. Although tissue is from an embryonic rat pup, the neurons have been shown to display mature receptor and electrophysiological profiles after 14 days in culture. The mixed neuronal culture was used in an adapted migration assay to study neuroregeneration and as an in vitro TBI model (Darbinyan et al., Methods Mol. Biol. 1078:45-54 (2013); Ali et al., High Content Screening with Primary Neurons. 2013 Oct 15. In: Sittampalam GS, Coussens NP, Brimacombe K, et al., editors. Assay Guidance Manual. Bethesda (MD): Eli Lilly & Company and the National Center for Advancing Translational Sciences (2004)).

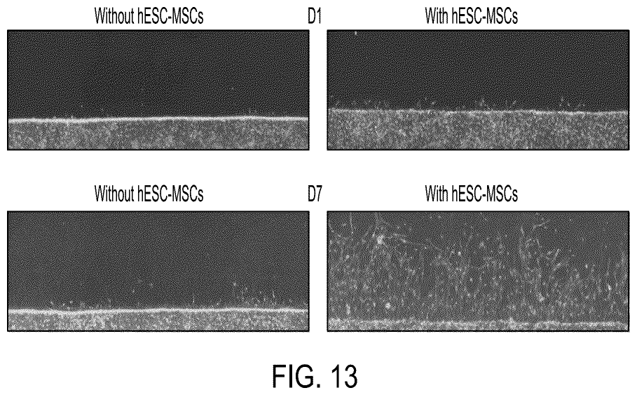

On day 0, the mixed neuronal culture was plated. On day 9, MARP12 cells that were frozen and thawed as described in Example 1 were plated in flasks for expansion. At Day 13, MARP12 cells were harvested and plated on transwell inserts for about a 10:1 ratio of neuron to MARP12 cells in MSC media. At day 14, two scratches were made per well in the mixed neuronal culture prepared as described above (Liang et al., Nat. Protoc. 2:329-333 (2007). The MSC media in the transwell was changed to neuronal media (Neurobasal™ Plus (Thermo Fisher); 1× Gentamicin; 1× GlutaMAX™ (ThermoFisher); 1× B27™ Plus (Thermo Fisher)) to remove all traces of serum, and the transwell inserts containing MARP12 cells were added to wells containing the mixed neuronal cultures. As shown in FIG. 13, co-culture with MARP12 (hESC-MSCs or HMC) encouraged neurite outgrowth and increased migration.

RNA-seq data can also show that the presence of the co-cultured HMCs and/or HMC-EVs can affect gene expression in the neurons. Neurons are dissociated from the cortex of brains of E18 Sprague-Dawley rats and plated at a density of 1.2×10⁶ cells per well on 6-well BioFlex culture plates (FlexCell Int.) that are coated with poly-D-lysine (Sigma). The neurons are supplemented with Neurobasal Plus/B27 Plus media (Gibco) and maintained for 14 days in vitro (DIV) at 37° C. in a humidified CO₂ incubator. Half media changes are performed every 3 days. For HMC treatment, HMCs are cultured for 4 days in α-MEM media (α-MEM (Hyclone) with 1× GlutaMAX (Gibco), 1×MEM-NEAA (Gibco), and Pen-strep (Gibco)) and then harvested and plated on transwell inserts (Corning) at a density of 1.2×10⁵ cells per insert. After one day in culture, the α-MEM media is changed to Neurobasal Plus/B27 Plus media for 1 hour, and the inserts are then added to the 6-well plates containing the neurons at DIV14. For EV treatment, EVs were purified from HMCs (HMC-EVs) by tangential flow filtration. HMC-EVs are added to the plates containing the neurons. TNF-α is then added at a concentration of 100 ng/mL where appropriate and the plates are then placed on the FlexCell FX-6000. The culture is subjected to 15%-0/o stretching oscillations (15%, 12.5%, 10%, 7.5%, 5%, 2.5%, and 0% cycles) overnight. The neurons are then removed from the BioFlex plate, pelleted, washed with PBS, and subjected to RNA isolation via the RNeasy Mini Kit (Qiagen). RNA (300 ng) is then submitted to BGI Americas for RNAseq analysis, and data is analyzed by Rosalind software (https://rosalind.onramp.bio/). Cutadapt is used to trim the reads, and FastQC is used to assess quality scores. STAR is used to align the reads to the Rattus norvegicus genome build rn5. HTseq is used to quantify the individual sample reads, and they are normalized via Relative Log Expression (RLE) using DESeq2 R library.

### Example 5—In Vivo Neonatal Hypoxia-Ischemia Model of Cerebral Palsy

The HMCs of the presently disclosed subject matter were tested in an in vivo neonatal hypoxia-ischemia (HI) model of cerebral palsy. HMCs used were MARP12 cells described in Example 1 that were thawed and passaged as passage 5 (P5) cells for four days upon which time, the cells were harvested, rinsed and formulated for injection. To establish the in vivo model for cerebral palsy, the common carotid artery in post-natal day (PND) 7 Sprague Dawley male rat pups was ligated to induce ischemia. Following recovery, pups were subjected to a hypoxic episode, followed by normoxia for 25 additional minutes. Pups in the sham control group received an equivalent exposure, except that normoxia rather than hypoxia was presented. At 7 days following surgery and hypoxic exposure (i.e. PND14), pups were humanely euthanized, with blood, cerebrospinal fluid (CSF), and brain tissue harvested for further testing. The pups were treated according to Table 2.

TABLE 2
Treatment Groups

| | | Maximum # | |
|---|---|---|---|
| Group | Treatment | per Group | Purpose |
| Lot B | HI | 8 | Test |
| | MARP12 | | article |
| | 1 × 10⁶ cells 6 hours post-hypoxia via | | |
| | IP injection | | |
| HI | HI, Vehicle Control | 8 | Control |
| Sham | Sham Control | 8 | Control |

### End Points Assessed

CSF and blood used for ELISAs for inflammatory panel and others depending on amount of sample.

Brain tissue analyzed for:
Cell death—TUNEL;
Infarct volume—H&E;
Iba-1—microglial activation in peri-infarct tissue;
GFAP—Astrocyte activation in peri-infarct tissue;
Olig2—Oligodendrocyte precursor cells in hippocampus
MBP—Myelin Basic Protein for mature oligodendrocytes in corpus callosum; and hippocampus.

### Results

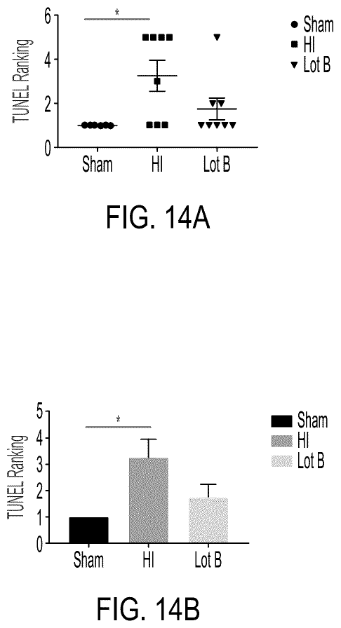

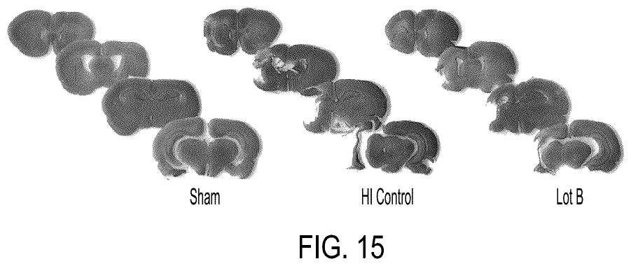

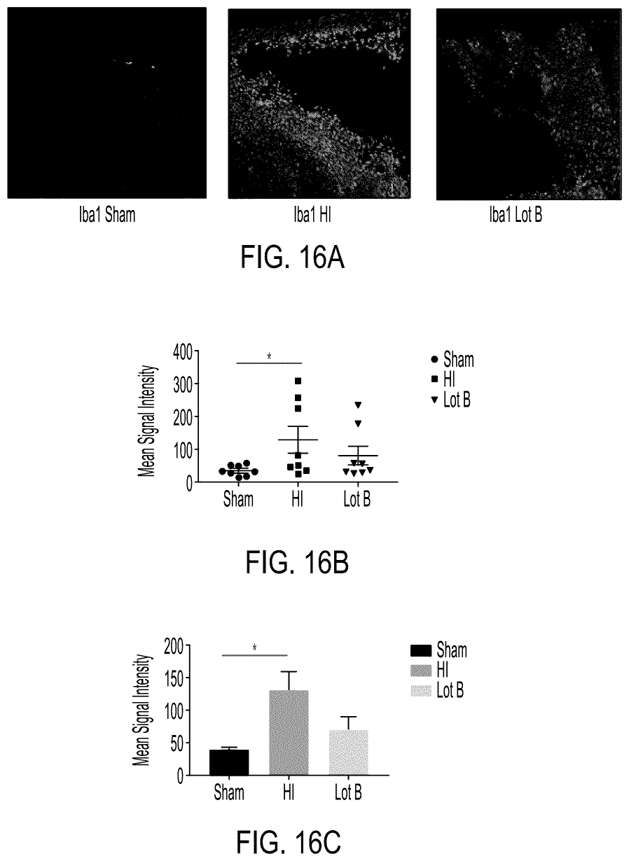

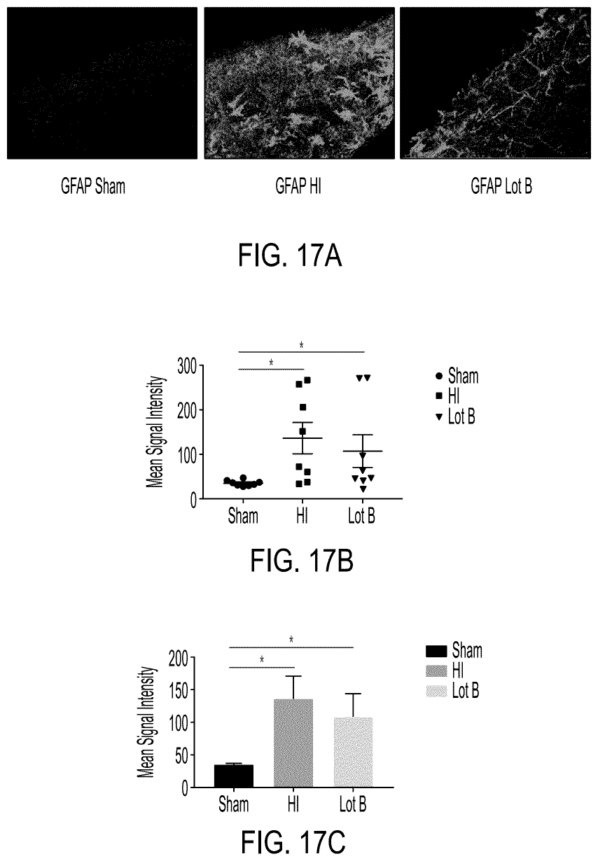

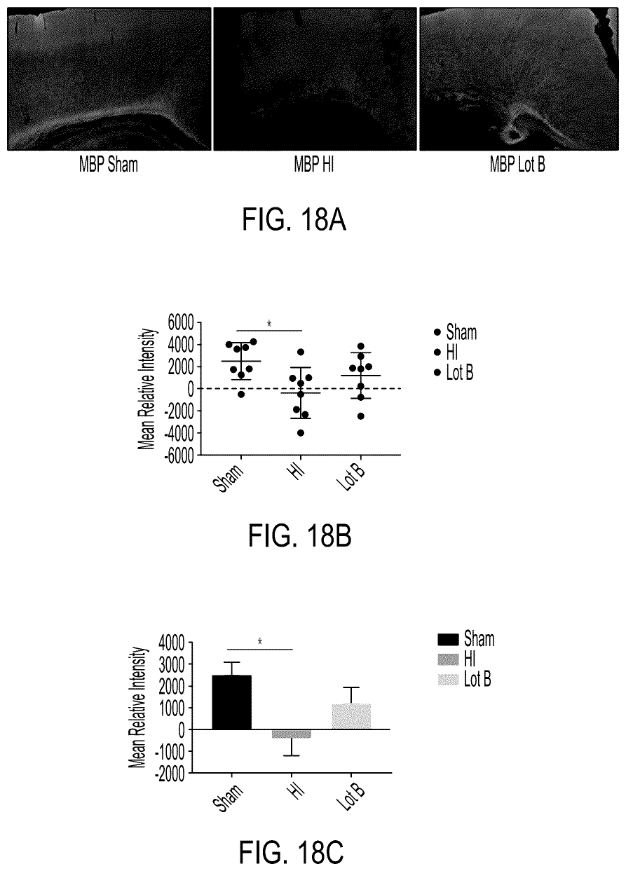

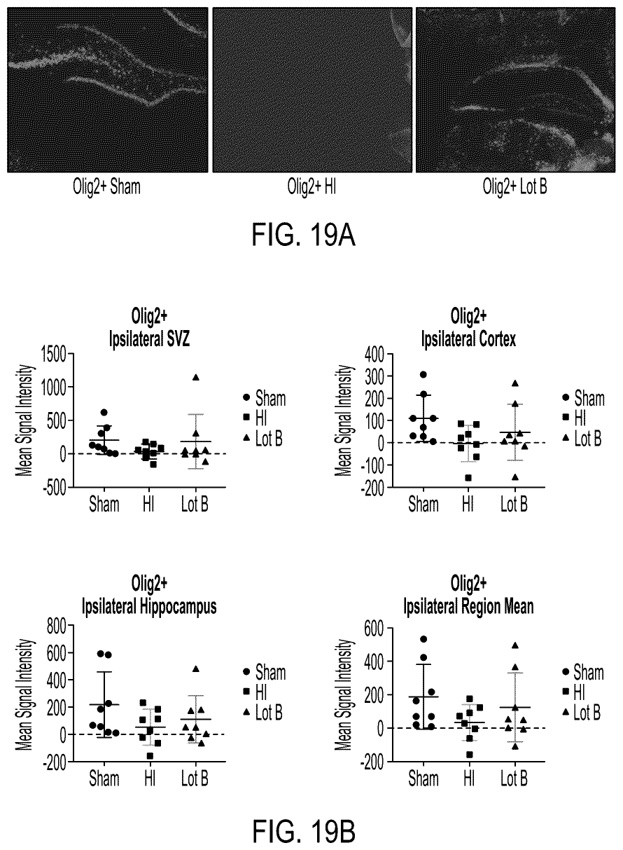

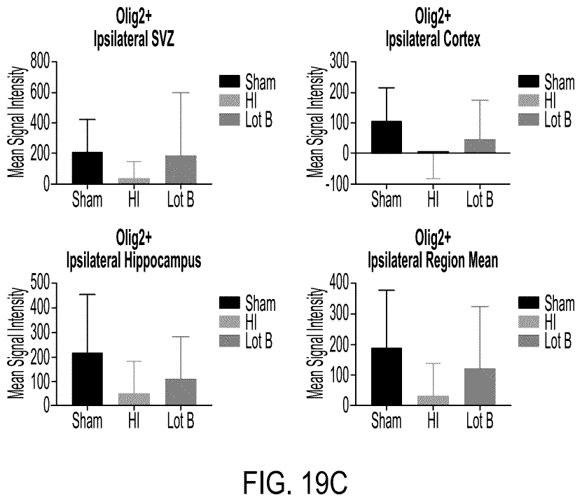

TUNEL staining as shown in FIGS. 14A-B suggests a neuroprotective effect by MARPS12 (Lot B) with reduced cell death. Further, H&E staining as shown in FIG. 15 suggests a neuroprotective effect by MARPS12 (Lot B) with reduced lesion size. A reduction in microglial activation via Iba-1 staining as shown in FIGS. 16A-C suggests an anti-inflammatory effect by MARPS12 (Lot B). A mild reduction in astrocyte activation via GFAP staining as shown in FIGS. 17A-C also suggests an anti-inflammatory effect by MARPS12 (Lot B). Preservation of myelin in the corpus callosum via MBP staining as shown in FIGS. 18A-C suggests a beneficial role of MARPS12 on oligodendrocytes. Moreover, FIGS. 19A-C suggest that Olig2 expression is partially rescued by administration of MARPS12.

These results support the use of HMCs in the treatment of cerebral palsy.

### Example 6—RNAseq Analysis of HMC Vs BM-MSC Vs UCB-MSC

HMCs were generated from the same bank of frozen hemangioblasts described in Example 1. Three separate lots of HMC were generated and passaged up to five passages (P5) according to the method described in Example 1. RNA seq analysis was performed on the three lots of HMC under basal conditions. MSCs isolated from bone marrow (BM-MSCs) (9 lots) and umbilical cord blood (UCB-MSCs) (9 lots) under basal conditions were used as controls.

Table 3 shows genes that were more highly expressed in the HMCs compared with BM-MSCs. Table 4 shows genes that were more highly expressed in BM-MSCs compared with the HMCs. Table 5 shows genes that were more highly expressed in HMCs compared with UCB-MSCs. Table 6 shows genes that were more highly expressed in UCB-MSCs compared with the HMCs. HMCs of the presently disclosed subject matter may be selected or purified based on any of the genes that are differentially expressed.

TABLE 3
Genes more highly expressed in HMCs compared with BM-MSCs

| | | | Log | |
|---|---|---|---|---|
| Gene | | Fold | Fold | |
| Name | Description | Change | Change | p-Adj |
| KCNN2 | potassium channel_calcium activated | 3376.7 | 11.7214 | 9.68E−96 |
| | intermediate/small conductance subfamily | | | |
| | N alpha_member 2 | | | |
| GATA4 | GATA binding protein 4 | 3374.36 | 11.7204 | 3.92E−74 |
| FAR2P1 | fatty acyl CoA reductase 2 pseudogene 1 | 2722.47 | 11.4107 | 2.85E−33 |
| GATA3 | GATA binding protein 3 | 2000.99 | 10.9665 | 9.13E−69 |
| NKX2-5 | NK2 homeobox 5 | 1763.59 | 10.7843 | 1.21E−69 |
| VAT1L | vesicle amine transport 1-like | 1436.96 | 10.4888 | 1.73E−168 |
| NRK | Nik related kinase | 1233.89 | 10.269 | 1.08E−36 |
| NETO1 | neuropilin (NRP) and tolloid (TLL)-like 1 | 1185.6 | 10.2114 | 9.67E−53 |
| BCHE | butyrylcholinesterase | 1128.82 | 10.1406 | 1.24E−46 |
| OCA2 | oculocutaneous albinism II | 1052.28 | 10.0393 | 5.00E−52 |
| GABRA5 | gamma-aminobutyric acid (GABA) A | 1034.77 | 10.0151 | 7.33E−112 |
| | receptor_alpha 5 | | | |
| DPPA4 | developmental pluripotency associated 4 | 1029.48 | 10.0077 | 6.22E−74 |
| KIF26A | kinesin family member 26A | 990.004 | 9.95129 | 7.81E−55 |
| RELN | reelin | 942.435 | 9.88025 | 1.16E−43 |
| LOC440416 | NA | 908.838 | 9.82788 | 1.42E−77 |
| SNCA | synuclein_alpha (non A4 component of | 880.69 | 9.78249 | 5.86E−40 |
| | amyloid precursor) | | | |
| GABRB1 | gamma-aminobutyric acid (GABA) A | 830.623 | 9.69805 | 1.47E−40 |
| | receptor_beta 1 | | | |
| SNRPN | small nuclear ribonucleoprotein polypeptide | 778.66 | 9.60485 | 3.61E−42 |
| | N | | | |
| CACNG4 | calcium channel_voltage-dependent_gamma | 757.788 | 9.56565 | 2.68E−56 |
| | subunit 4 | | | |
| LRRTM1 | leucine rich repeat transmembrane | 717.547 | 9.48693 | 4.54E−44 |
| | neuronal 1 | | | |
| LINGO2 | leucine rich repeat and Ig domain | 620.437 | 9.27714 | 4.01E−40 |
| | containing 2 | | | |
| TNNT2 | troponin T type 2 (cardiac) | 594.602 | 9.21578 | 1.04E−36 |
| ZNF804A | zinc finger protein 804A | 586.802 | 9.19673 | 6.40E−56 |
| ST6GAL2 | ST6 beta-galactosamide alpha-2_6- | 576.929 | 9.17225 | 7.18E−88 |
| | sialyltranferase 2 | | | |
| COL4A5 | collagen_type IV_alpha 5 | 576.757 | 9.17182 | 2.11E−82 |
| LIN28B | lin-28 homolog B (C. elegans) | 563.605 | 9.13854 | 2.92E−39 |
| MMP9 | matrix metallopeptidase 9 | 554.502 | 9.11505 | 1.92E−42 |
| SLC7A2 | solute carrier family 7 (cationic amino | 520.325 | 9.02327 | 3.31E−149 |
| | acid transporter_y+ system)_member 2 | | | |
| COL4A6 | collagen_type IV_alpha 6 | 497.261 | 8.95786 | 1.25E−97 |
| FENDRR | FOXF1 adjacent non-coding developmental | 488.058 | 8.93091 | 1.86E−46 |
| | regulatory RNA | | | |
| DSC2 | desmocollin 2 | 478.415 | 8.90212 | 2.20E−39 |
| KCTD8 | potassium channel tetramerization domain | 459.857 | 8.84504 | 3.51E−38 |
| | containing 8 | | | |
| ARAP2 | ArfGAP with RhoGAP domain_ankyrin | 455.472 | 8.83122 | 4.05E−38 |
| | repeat and PH domain 2 | | | |
| DIO2 | deiodinase_iodothyronine_type II | 450.443 | 8.8152 | 1.78E−98 |
| CDH10 | cadherin 10_type 2 (T2-cadherin) | 448.881 | 8.81019 | 7.16E−25 |
| SHC3 | SHC (Src homology 2 domain containing) | 447.61 | 8.8061 | 3.60E−90 |
| | transforming protein 3 | | | |
| SULT1E1 | sulfotransferase family 1E_estrogen- | 447.155 | 8.80463 | 2.93E−34 |
| | preferring_member 1 | | | |
| CPXM1 | carboxypeptidase X (M14 family)_member | 445.688 | 8.79989 | 1.94E−75 |
| | 1 | | | |
| FGF20 | fibroblast growth factor 20 | 428.96 | 8.7447 | 9.75E−34 |
| LINC00890 | long intergenic non-protein coding RNA 890 | 382.729 | 8.58018 | 1.14E−32 |
| BAI3 | adhesion G protein-coupled receptor B3 | 364.764 | 8.51082 | 8.84E−35 |
| L1CAM | L1 cell adhesion molecule | 361.67 | 8.49853 | 1.36E−94 |
| CACNG8 | calcium channel_voltage-dependent_gamma | 359.757 | 8.49088 | 1.88E−29 |
| | subunit 8 | | | |
| SULT1C4 | sulfotransferase family_cytosolic_1C_member | 324.225 | 8.34085 | 4.13E−29 |
| | 4 | | | |
| TRIM55 | tripartite motif containing 55 | 319.183 | 8.31824 | 9.79E−22 |
| HOXB13 | homeobox B13 | 313.091 | 8.29044 | 4.19E−32 |
| DSG2 | desmoglein 2 | 309.567 | 8.27411 | 3.18E−14 |
| ELFN2 | extracellular leucine-rich repeat and | 301.134 | 8.23426 | 1.62E−92 |
| | fibronectin type III domain containing 2 | | | |
| CTD-2297D10.2 | uncharacterized LOC101929176 | 300.946 | 8.23336 | 5.57E−22 |
| TRPC5 | transient receptor potential cation | 297.627 | 8.21736 | 6.17E−23 |
| | channel_subfamily C_member 5 | | | |
| WT1 | Wilms tumor 1 | 297.142 | 8.21501 | 4.53E−32 |
| TMEM63C | transmembrane protein 63C | 296.544 | 8.2121 | 1.88E−36 |
| RERG | RAS-like_estrogen-regulated_growth | 292.372 | 8.19166 | 3.31E−32 |
| | inhibitor | | | |
| CCND2 | cyclin D2 | 288.586 | 8.17286 | 2.31E−48 |
| NKX2-3 | NK2 homeobox 3 | 287.642 | 8.16813 | 4.09E−28 |
| SAMD5 | sterile alpha motif domain containing 5 | 281.787 | 8.13846 | 2.29E−79 |
| STMN2 | stathmin 2 | 281.654 | 8.13778 | 7.49E−14 |
| TMEM200C | transmembrane protein 200C | 277.722 | 8.1175 | 9.71E−27 |
| SOX17 | SRY (sex determining region Y)-box 17 | 277.509 | 8.11639 | 2.49E−29 |
| MGAT3 | mannosyl (beta-1_4-)-glycoprotein beta- | 269.263 | 8.07287 | 3.27E−96 |
| | 1_4-N-acetylglucosaminyltransferase | | | |
| FLT1 | fms-related tyrosine kinase 1 | 266.319 | 8.05701 | 1.95E−173 |
| NKAIN4 | Na+/K+ transporting ATPase interacting 4 | 260.054 | 8.02267 | 3.36E−39 |
| SYTL5 | synaptotagmin-like 5 | 257.406 | 8.0079 | 8.81E−79 |
| MDGA2 | MAM domain containing | 252.998 | 7.98298 | 4.70E−26 |
| | glycosylphosphatidylinositol anchor 2 | | | |
| GATA3-AS1 | GATA3 antisense RNA 1 | 249.784 | 7.96454 | 3.99E−22 |
| LGI1 | leucine-rich_glioma inactivated 1 | 248.088 | 7.95471 | 5.19E−26 |
| PKP2 | plakophilin 2 | 247.539 | 7.95151 | 2.82E−15 |
| KLHL4 | kelch-like family member 4 | 238.045 | 7.89509 | 3.70E−63 |
| GPR143 | G protein-coupled receptor 143 | 235.692 | 7.88076 | 5.07E−44 |
| ADAMTS18 | ADAM metallopeptidase with | 219.386 | 7.77733 | 5.32E−25 |
| | thrombospondin type 1 motif_18 | | | |
| CHRM2 | cholinergic receptor_muscarinic 2 | 218.008 | 7.76824 | 1.34E−14 |
| TMEM40 | transmembrane protein 40 | 216.144 | 7.75585 | 2.22E−25 |
| NIPAL4 | NIPA-like domain containing 4 | 213.309 | 7.7368 | 6.44E−119 |
| SEMA3D | sema domain_immunoglobulin domain | 212.776 | 7.73319 | 4.51E−37 |
| | (Ig)_short basic | | | |
| | domain_secreted_(semaphorin) 3D | | | |
| PHOX2A | paired-like homeobox 2a | 212.508 | 7.73137 | 1.17E−27 |
| PRAC1 | prostate cancer susceptibility candidate 1 | 200.695 | 7.64886 | 3.28E−20 |
| CSMD3 | CUB and Sushi multiple domains 3 | 191.196 | 7.57891 | 4.33E−23 |
| B3GAT1 | beta-1_3-glucuronyltransferase 1 | 189.606 | 7.56686 | 7.70E−26 |
| TRIM58 | tripartite motif containing 58 | 189.244 | 7.5641 | 4.32E−32 |
| ANO4 | anoctamin 4 | 186.743 | 7.54491 | 2.59E−41 |
| GPR20 | G protein-coupled receptor 20 | 186.668 | 7.54433 | 9.67E−22 |
| EEF1A2 | eukaryotic translation elongation factor 1 | 186.624 | 7.54399 | 9.79E−37 |
| | alpha 2 | | | |
| HOXD11 | homeobox D11 | 184.825 | 7.53002 | 4.91E−37 |
| LHX1 | LIM homeobox 1 | 183.385 | 7.51873 | 6.08E−21 |
| DCC | DCC netrin 1 receptor | 177.536 | 7.47197 | 2.29E−36 |
| SHC2 | SHC (Src homology 2 domain containing) | 177.418 | 7.47101 | 3.45E−36 |
| | transforming protein 2 | | | |
| FIRRE | firre intergenic repeating RNA element | 175.85 | 7.4582 | 2.53E−19 |
| HAND2-AS1 | HAND2 antisense RNA 1 (head to head) | 173.707 | 7.44051 | 7.94E−44 |
| MAB21L2 | mab-21-like 2 (C. elegans) | 171.99 | 7.42618 | 5.59E−25 |
| TMC6 | transmembrane channel-like 6 | 171.467 | 7.42179 | 1.23E−42 |
| KDR | kinase insert domain receptor | 171.259 | 7.42004 | 8.29E−26 |
| C2CD4C | C2 calcium-dependent domain containing | 167.398 | 7.38714 | 1.53E−42 |
| | 4C | | | |
| CXXC4 | CXXC finger protein 4 | 164.691 | 7.36362 | 1.29E−19 |
| LGR5 | leucine-rich repeat containing G protein- | 163.206 | 7.35055 | 4.04E−44 |
| | coupled receptor 5 | | | |
| DSC3 | desmocollin 3 | 162.352 | 7.34298 | 1.77E−10 |
| IL1RAPL1 | interleukin 1 receptor accessory protein- | 158.417 | 7.30758 | 2.79E−17 |
| | like 1 | | | |
| VANGL2 | VANGL planar cell polarity protein 2 | 153.694 | 7.26392 | 2.36E−55 |
| ABCB1 | ATP-binding cassette_sub-family B | 147.802 | 7.20752 | 3.07E−26 |
| | (MDR/TAP)_member 1 | | | |
| AADAC | arylacetamide deacetylase | 140.148 | 7.13081 | 7.12E−17 |
| FSTL5 | follistatin-like 5 | 139.259 | 7.12163 | 2.68E−15 |
| MED15P9 | mediator complex subunit 15 pseudogene 9 | 138.438 | 7.1131 | 5.39E−10 |
| GCNT2 | glucosaminyl (N-acetyl) transferase 2_I- | 133.285 | 7.05837 | 1.16E−15 |
| | branching enzyme (I blood group) | | | |
| SULT1B1 | sulfotransferase | 132.429 | 7.04907 | 8.14E−21 |
| | family_cytosolic_1B_member 1 | | | |
| GPR87 | G protein-coupled receptor 87 | 132.396 | 7.04872 | 3.45E−10 |
| LIN28A | lin-28 homolog A (C. elegans) | 130.54 | 7.02835 | 7.46E−19 |
| KRT8 | keratin 8_type II | 130.494 | 7.02784 | 2.19E−255 |
| SLC35F3 | solute carrier family 35_member F3 | 129.889 | 7.02114 | 4.02E−18 |
| MYRF | myelin regulatory factor | 127.908 | 6.99896 | 8.88E−97 |
| TIE1 | tyrosine kinase with immunoglobulin- | 125.933 | 6.97651 | 3.53E−48 |
| | like and EGF-like domains 1 | | | |
| FAT3 | FAT atypical cadherin 3 | 125.595 | 6.97264 | 2.69E−61 |
| C8orf49 | chromosome 8 open reading frame 49 | 119.914 | 6.90586 | 2.72E−18 |
| GABRA4 | gamma-aminobutyric acid (GABA) A | 119.403 | 6.89969 | 1.79E−15 |
| | receptor_alpha 4 | | | |
| PCDH7 | protocadherin 7 | 119.262 | 6.89799 | 3.97E−83 |
| ST6GALNAC3 | ST6 (alpha-N-acetyl-neuraminyl-2_3-beta- | 118.478 | 6.88848 | 2.53E−23 |
| | galactosy1-1_3)-N-acetylgalactosaminide | | | |
| | alpha-2_6-sialyltransferase 3 | | | |
| PPP2R2B | protein phosphatase 2_regulatory subunit | 118.228 | 6.88543 | 3.56E−74 |
| | B_beta | | | |
| C6orf141 | chromosome 6 open reading frame 141 | 117.977 | 6.88236 | 2.95E−18 |
| SFMBT2 | Scm-like with four mbt domains 2 | 116.043 | 6.85851 | 2.63E−33 |
| SPINK5 | serine peptidase inhibitor_Kazal type 5 | 115.386 | 6.85032 | 1.10E−08 |
| SLC6A15 | solute carrier family 6 (neutral amino | 112.26 | 6.8107 | 6.07E−17 |
| | acid transporter)_member 15 | | | |
| FXYD6 | FXYD domain containing ion transport | 108.606 | 6.76296 | 1.75E−17 |
| | regulator 6 | | | |
| DNAH11 | dynein_axonemal_heavy chain 11 | 107.843 | 6.75279 | 8.79E−60 |
| SCG2 | secretogranin II | 106.966 | 6.74101 | 4.54E−67 |
| SEMA3E | sema domain_immunoglobulin domain | 106.595 | 6.736 | 9.68E−18 |
| | (Ig)_short basic | | | |
| | domain_secreted_(semaphorin) 3E | | | |
| GAL | galanin/GMAP prepropeptide | 105.543 | 6.72169 | 4.15E−52 |
| NPY | neuropeptide Y | 104.525 | 6.70771 | 1.51E−15 |
| KCNH2 | potassium channel_voltage gated eag | 102.046 | 6.67308 | 9.32E−33 |
| | related subfamily H_member 2 | | | |
| SYTL1 | synaptotagmin-like 1 | 99.8984 | 6.64239 | 1.73E−47 |
| HOPX | HOP homeobox | 98.9453 | 6.62856 | 1.74E−17 |
| GPR37 | G protein-coupled receptor 37 (endothelin | 98.1407 | 6.61678 | 8.32E−36 |
| | receptor type B-like) | | | |
| CLSTN2 | calsyntenin 2 | 97.1573 | 6.60225 | 6.01E−51 |
| SLCO4A1 | solute carrier organic anion transporter | 96.0211 | 6.58528 | 3.70E−20 |
| | family_member 4A1 | | | |
| LUZP2 | leucine zipper protein 2 | 95.3037 | 6.57446 | 1.86E−13 |
| ERP27 | endoplasmic reticulum protein 27 | 87.6213 | 6.45321 | 5.22E−15 |
| TAGLN3 | transgelin 3 | 87.0661 | 6.44404 | 8.10E−50 |
| CACNA1H | calcium channel_voltage-dependent_T | 86.7024 | 6.438 | 2.39E−85 |
| | type_alpha 1H subunit | | | |
| NOVA1 | neuro-oncological ventral antigen 1 | 85.9586 | 6.42557 | 1.21E−09 |
| IGSF3 | immunoglobulin superfamily_member 3 | 85.2324 | 6.41333 | 5.56E−38 |
| P2RY14 | purinergic receptor P2Y_G-protein | 84.4116 | 6.39937 | 7.54E−13 |
| | coupled_14 | | | |
| SLC5A4 | solute carrier family 5 (glucose | 83.7995 | 6.38887 | 6.99E−15 |
| | activated ion channel)_member 4 | | | |
| NDST3 | N-deacetylase/N-sulfotransferase | 83.6463 | 6.38623 | 3.11E−20 |
| | (heparan glucosaminyl) 3 | | | |
| HOXD10 | homeobox D10 | 83.2622 | 6.37959 | 6.03E−24 |
| FOXF1 | forkhead box F1 | 82.2857 | 6.36257 | 9.91E−08 |
| HAND1 | heart and neural crest derivatives | 80.2556 | 6.32653 | 1.20E−12 |
| | expressed 1 | | | |
| CTTNBP2 | cortactin binding protein 2 | 77.8222 | 6.28211 | 1.15E−09 |
| ADAMTS16 | ADAM metallopeptidase with | 77.6573 | 6.27905 | 1.53E−57 |
| | thrombospondin type 1 motif_16 | | | |
| ELOVL2 | ELOVL fatty acid elongase 2 | 77.076 | 6.26821 | 6.48E−39 |
| HOXB9 | homeobox B9 | 76.7162 | 6.26146 | 2.85E−09 |
| PLCXD3 | phosphatidylinositol-specific phospholipase | 74.8868 | 6.22664 | 3.68E−13 |
| | C_X domain containing 3 | | | |
| SCN5A | sodium channel_voltage gated_type V | 74.3881 | 6.217 | 3.97E−24 |
| | alpha subunit | | | |
| TRIL | TLR4 interactor with leucine-rich repeats | 73.8563 | 6.20665 | 1.44E−14 |
| HIST1H2BH | histone cluster 1_H2bh | 73.8405 | 6.20634 | 2.65E−21 |
| MYL7 | myosin_light chain 7_regulatory | 73.5177 | 6.20002 | 3.16E−17 |
| TEPP | testis_prostate and placenta expressed | 73.0296 | 6.19041 | 2.06E−15 |
| HOXB8 | homeobox B8 | 73.018 | 6.19018 | 6.99E−44 |
| LIPG | lipase_endothelial | 72.8496 | 6.18685 | 1.62E−38 |
| SLCO6A1 | solute carrier organic anion transporter | 72.6328 | 6.18255 | 3.74E−10 |
| | family_member 6A1 | | | |
| IGDCC3 | immunoglobulin superfamily_DCC | 72.6258 | 6.18241 | 1.28E−22 |
| | subclass_member 3 | | | |
| GABRG3 | gamma-aminobutyric acid (GABA) A | 72.1476 | 6.17288 | 4.13E−11 |
| | receptor_gamma 3 | | | |
| GRIA1 | glutamate receptor_ionotropic_AMPA 1 | 71.9404 | 6.16873 | 1.08E−37 |
| C8orf4 | chromosome 8 open reading frame 4 | 71.2481 | 6.15478 | 9.53E−24 |
| FABP4 | fatty acid binding protein 4_adipocyte | 70.9554 | 6.14884 | 1.96E−09 |
| PLEKHG4B | pleckstrin homology domain containing_family | 70.7746 | 6.14516 | 8.93E−52 |
| | G (with RhoGef domain) member 4B | | | |
| IP6K3 | inositol hexakisphosphate kinase 3 | 69.7939 | 6.12503 | 1.34E−16 |
| PDE9A | phosphodiesterase 9A | 67.1097 | 6.06845 | 1.00E−15 |
| KLHDC8A | kelch domain containing 8A | 66.2124 | 6.04903 | 1.29E−09 |
| FLJ16779 | uncharacterized LOC100192386 | 65.8988 | 6.04218 | 5.66E−07 |
| CCDC160 | coiled-coil domain containing 160 | 64.6832 | 6.01532 | 1.22E−11 |
| SPP1 | secreted phosphoprotein 1 | 63.3767 | 5.98588 | 2.40E−37 |
| PCDH17 | protocadherin 17 | 63.0227 | 5.9778 | 1.49E−10 |
| HOTTIP | HOXA distal transcript antisense RNA | 62.4396 | 5.96439 | 3.67E−19 |
| OXTR | oxytocin receptor | 62.3043 | 5.96126 | 1.14E−36 |
| SH2D3C | SH2 domain containing 3C | 62.2667 | 5.96039 | 2.83E−68 |
| USP43 | ubiquitin specific peptidase 43 | 61.9104 | 5.95211 | 1.62E−26 |
| KC6 | keratoconus gene 6 | 61.6005 | 5.94487 | 4.37E−07 |
| CACNG7 | calcium channel_voltage-dependent_gamma | 61.5198 | 5.94298 | 1.74E−114 |
| | subunit 7 | | | |
| SLC44A5 | solute carrier family 44_member 5 | 60.9756 | 5.93016 | 4.59E−63 |
| COL18A1 | collagen_type XVIII_alpha 1 | 60.1278 | 5.90996 | 0.00E+00 |
| LINC00491 | long intergenic non-protein coding RNA 491 | 60.0324 | 5.90767 | 6.94E−12 |
| TBX1 | T-box 1 | 60.0149 | 5.90725 | 1.38E−30 |
| GALNT14 | polypeptide N- | 59.4424 | 5.89342 | 9.44E−16 |
| | acetylgalactosaminyltransferase 14 | | | |
| CLEC1A | C-type lectin domain family 1_member A | 59.3592 | 5.8914 | 1.45E−09 |
| CALY | calcyon neuron-specific vesicular protein | 59.309 | 5.89018 | 1.54E−21 |
| CD93 | CD93 molecule | 58.2498 | 5.86418 | 9.02E−15 |
| HIF3A | hypoxia inducible factor 3_alpha subunit | 58.2328 | 5.86376 | 2.36E−19 |
| LPAR4 | lysophosphatidic acid receptor 4 | 58.2304 | 5.8637 | 4.90E−18 |
| TBX20 | T-box 20 | 57.7408 | 5.85152 | 1.78E−06 |
| TNRC6C-AS1 | TNRC6C antisense RNA 1 | 57.652 | 5.8493 | 4.12E−13 |
| CHMP4C | charged multivesicular body protein 4C | 56.3561 | 5.8165 | 1.76E−18 |
| CADM1 | cell adhesion molecule 1 | 56.3186 | 5.81554 | 1.11E−89 |
| SDK1 | sidekick cell adhesion molecule 1 | 55.5517 | 5.79576 | 9.60E−52 |
| MMP10 | matrix metallopeptidase 10 | 55.3001 | 5.78921 | 4.28E−11 |
| MERTK | MER proto-oncogene_tyrosine kinase | 55.1428 | 5.7851 | 2.56E−26 |
| DPY19L2P1 | DPY19L2 pseudogene 1 | 55.0725 | 5.78326 | 1.10E−82 |
| GPRC5B | G protein-coupled receptor_class C_group | 54.6061 | 5.77099 | 2.76E−17 |
| | 5_member B | | | |
| VWDE | von Willebrand factor D and EGF domains | 54.0424 | 5.75602 | 1.37E−13 |
| CIDEA | cell death-inducing DFFA-like effector a | 53.9432 | 5.75337 | 4.10E−11 |
| RASGRF1 | Ras protein-specific guanine nucleotide- | 53.6193 | 5.74468 | 1.80E−21 |
| | releasing factor 1 | | | |
| CACNG6 | calcium channel_voltage-dependent_gamma | 53.5476 | 5.74275 | 8.41E−09 |
| | subunit 6 | | | |
| FAM189A1 | family with sequence similarity 189_member | 53.2323 | 5.73423 | 5.88E−18 |
| | A1 | | | |
| IL2RB | interleukin 2 receptor_beta | 52.6777 | 5.71912 | 9.40E−31 |
| C1orf106 | chromosome 1 open reading frame 106 | 52.1675 | 5.70508 | 8.35E−35 |
| CRHBP | corticotropin releasing hormone binding | 52.0357 | 5.70143 | 5.66E−12 |
| | protein | | | |
| HBD | hemoglobin_delta | 51.5443 | 5.68774 | 4.43E−11 |
| MGAT4C | MGAT4 family_member C | 49.6272 | 5.63306 | 4.86E−10 |
| RBM20 | RNA binding motif protein 20 | 49.1418 | 5.61888 | 3.22E−14 |
| KCNA1 | potassium channel_voltage gated shaker | 49.1238 | 5.61835 | 9.02E−12 |
| | related subfamily A_member 1 | | | |
| SEMA3A | sema domain_immunoglobulin domain | 48.3221 | 5.59461 | 4.17E−74 |
| | (Ig)_short basic | | | |
| | domain_secreted_(semaphorin) 3A | | | |
| SORCS3 | sortilin-related VPS10 domain | 48.1716 | 5.59011 | 3.21E−08 |
| | containing receptor 3 | | | |
| SLC22A31 | solute carrier family 22_member 31 | 47.946 | 5.58334 | 8.45E−22 |
| ZCCHC16 | zinc finger_CCHC domain containing 16 | 47.7911 | 5.57867 | 2.49E−08 |
| SHISA3 | shisa family member 3 | 47.5212 | 5.5705 | 9.76E−18 |
| VGF | VGF nerve growth factor inducible | 47.2303 | 5.56164 | 2.03E−20 |
| CPVL | carboxypeptidase_vitellogenic-like | 47.0731 | 5.55683 | 3.13E−08 |
| FAM213A | family with sequence similarity | 46.8767 | 5.5508 | 2.15E−17 |
| | 213_member A | | | |
| HTR1D | 5-hydroxytryptamine (serotonin) receptor | 46.5442 | 5.54053 | 1.25E−28 |
| | 1D_G protein-coupled | | | |
| PCDHA12 | protocadherin alpha 12 | 45.8017 | 5.51733 | 7.82E−06 |
| NTSR1 | neurotensin receptor 1 (high affinity) | 44.7576 | 5.48406 | 6.68E−10 |
| FAM69B | family with sequence similarity 69_member | 43.6101 | 5.44659 | 2.53E−96 |
| | B | | | |
| LRRN4 | leucine rich repeat neuronal 4 | 42.0904 | 5.39542 | 3.90E−26 |
| LOC644919 | uncharacterized LOC644919 | 40.994 | 5.35734 | 1.75E−09 |
| COL9A3 | collagen_type IX_alpha 3 | 40.5677 | 5.34226 | 3.87E−50 |
| GIPC3 | GIPC PDZ domain containing | 40.4621 | 5.3385 | 4.22E−140 |
| | family_member 3 | | | |
| CYTL1 | cytokine-like 1 | 40.3604 | 5.33487 | 2.91E−20 |
| GBX2 | gastrulation brain homeobox 2 | 39.8398 | 5.31614 | 1.15E−07 |
| C2orf91 | chromosome 2 open reading frame 91 | 38.997 | 5.28529 | 7.95E−09 |
| TTLL6 | tubulin tyrosine ligase-like family | 38.9764 | 5.28453 | 1.48E−08 |
| | member 6 | | | |
| IFLTD1 | lamin tail domain containing 1 | 38.9187 | 5.28239 | 3.52E−12 |
| CECR2 | cat eye syndrome chromosome | 38.553 | 5.26877 | 3.66E−08 |
| | region_candidate 2 | | | |
| PDGFB | platelet-derived growth factor beta | 38.5383 | 5.26822 | 6.45E−21 |
| | polypeptide | | | |
| SSTR1 | somatostatin receptor 1 | 37.612 | 5.23312 | 1.10E−06 |
| RGS5 | regulator of G-protein signaling 5 | 37.382 | 5.22427 | 1.21E−127 |
| MMP23B | matrix metallopeptidase 23B | 37.1557 | 5.21551 | 2.07E−27 |
| ISL1 | ISL LIM homeobox 1 | 36.8768 | 5.20464 | 1.70E−14 |
| ABI3 | ABI family_member 3 | 36.724 | 5.19865 | 2.86E−20 |
| ZPLD1 | zona pellucida-like domain containing 1 | 36.7237 | 5.19864 | 2.13E−11 |
| PDE3B | phosphodiesterase 3B_cGMP-inhibited | 36.6545 | 5.19592 | 7.16E−22 |
| BEST3 | bestrophin 3 | 36.5693 | 5.19256 | 3.02E−12 |
| B4GALNT4 | beta-1_4-N-acetyl-galactosaminy1 | 36.2902 | 5.18151 | 1.97E−21 |
| | transferase 4 | | | |
| LRRC17 | leucine rich repeat containing 17 | 36.1996 | 5.1779 | 9.35E−27 |
| KCNA6 | potassium channel_voltage gated shaker | 36.0306 | 5.17115 | 1.91E−15 |
| | related subfamily A_member 6 | | | |
| NRXN3 | neurexin 3 | 36.0153 | 5.17054 | 4.93E−26 |
| MGC2889 | uncharacterized protein MGC2889 | 35.8955 | 5.16573 | 7.62E−08 |
| ADAMTS20 | ADAM metallopeptidase with | 35.2102 | 5.13792 | 1.38E−08 |
| | thrombospondin type 1 motif_20 | | | |
| HUNK | hormonally up-regulated Neu-associated | 34.6857 | 5.11627 | 3.50E−14 |
| | kinase | | | |
| MTUS1 | microtubule associated tumor suppressor 1 | 34.2018 | 5.096 | 3.01E−24 |
| LOC101929086 | NA | 34.028 | 5.08865 | 8.77E−07 |
| DACT2 | dishevelled-binding antagonist of beta- | 33.744 | 5.07656 | 1.56E−06 |
| | catenin 2 | | | |
| ACTG2 | actin_gamma 2_smooth muscle_enteric | 33.0521 | 5.04667 | 1.30E−11 |
| WNT2 | wingless-type MMTV integration site family | 32.8017 | 5.0357 | 8.54E−08 |
| | member 2 | | | |
| TTR | transthyretin | 32.3991 | 5.01788 | 2.02E−06 |
| SFRP1 | secreted frizzled-related protein 1 | 32.2615 | 5.01174 | 6.51E−40 |
| GRPR | gastrin-releasing peptide receptor | 32.2049 | 5.00921 | 3.28E−29 |
| CCDC88C | coiled-coil domain containing 88C | 32.1773 | 5.00797 | 8.98E−23 |
| LOC440910 | uncharacterized LOC440910 | 32.1351 | 5.00608 | 7.01E−06 |
| CYP2S1 | cytochrome P450_family | 32.1307 | 5.00588 | 3.24E−59 |
| | 2_subfamily S_polypeptide 1 | | | |
| LRRN1 | leucine rich repeat neuronal 1 | 32.0926 | 5.00417 | 1.17E−06 |
| C7 | complement component 7 | 32.0613 | 5.00276 | 2.19E−13 |
| NDRG2 | NDRG family member 2 | 32.0118 | 5.00053 | 1.08E−55 |
| ZDHHC8P1 | zinc finger_DHHC-type containing 8 | 31.9831 | 4.99924 | 3.17E−14 |
| | pseudogene 1 | | | |
| LRFN5 | leucine rich repeat and fibronectin | 31.9362 | 4.99712 | 8.06E−09 |
| | type III domain containing 5 | | | |
| NR0B1 | nuclear receptor subfamily 0_group | 31.7781 | 4.98996 | 1.35E−05 |
| | B_member 1 | | | |
| FAM105A | family with sequence similarity | 31.7613 | 4.9892 | 2.11E−17 |
| | 105_member A | | | |
| MMP1 | matrix metallopeptidase 1 | 31.7026 | 4.98653 | 3.12E−12 |
| GABRQ | gamma-aminobutyric acid (GABA) A | 31.1647 | 4.96184 | 4.24E−07 |
| | receptor_theta | | | |
| C9orf47 | chromosome 9 open reading frame 47 | 31.1247 | 4.95999 | 1.13E−14 |
| HAND2 | heart and neural crest derivatives | 30.8252 | 4.94604 | 7.86E−05 |
| | expressed 2 | | | |
| ARHGDIB | Rho GDP dissociation inhibitor (GDI) beta | 30.6697 | 4.93874 | 1.46E−162 |
| KCNMB4 | potassium channel subfamily M regulatory | 30.6622 | 4.93839 | 3.00E−36 |
| | beta subunit 4 | | | |
| LOC728392 | uncharacterized LOC728392 | 30.6522 | 4.93792 | 1.84E−102 |
| NUTM2F | NUT family member 2F | 30.1029 | 4.91183 | 3.45E−07 |
| GRIP1 | glutamate receptor interacting protein 1 | 30.0545 | 4.90951 | 8.20E−33 |
| AIM1L | absent in melanoma 1-like | 29.8554 | 4.89992 | 5.19E−08 |
| WT1-AS | WT1 antisense RNA | 29.8471 | 4.89952 | 8.31E−07 |
| PNMA3 | paraneoplastic Ma antigen 3 | 29.7352 | 4.8941 | 4.99E−14 |
| TPSG1 | tryptase gamma 1 | 29.473 | 4.88132 | 9.37E−08 |
| MOV10L1 | Mov10 RISC complex RNA helicase like 1 | 29.1231 | 4.86409 | 5.72E−36 |
| HOXD13 | homeobox D13 | 29.089 | 4.8624 | 1.15E−08 |
| KAL1 | anosmin 1 | 29.0122 | 4.85859 | 2.64E−42 |
| KNDC1 | kinase non-catalytic C-lobe domain | 28.744 | 4.84519 | 3.37E−33 |
| | (KIND) containing 1 | | | |
| ADAM23 | ADAM metallopeptidase domain 23 | 28.5026 | 4.83302 | 8.82E−19 |
| TYRP1 | tyrosinase-related protein 1 | 28.363 | 4.82594 | 1.51E−22 |
| SP140 | SP140 nuclear body protein | 28.3 | 4.82273 | 3.34E−28 |
| LOC100652770 | NA | 28.1835 | 4.81678 | 1.28E−05 |
| ZNF467 | zinc finger protein 467 | 28.1178 | 4.81341 | 7.48E−14 |
| GPR115 | adhesion G protein-coupled receptor F4 | 27.9129 | 4.80286 | 1.95E−08 |
| PNMT | phenylethanolamine N-methyltransferase | 27.911 | 4.80276 | 1.28E−05 |
| LINC00648 | long intergenic non-protein coding RNA 648 | 27.9067 | 4.80254 | 8.86E−06 |
| FAM95C | family with sequence similarity 95_member | 27.6934 | 4.79147 | 1.23E−06 |
| | C | | | |
| LOC101928340 | NA | 27.633 | 4.78832 | 1.36E−05 |
| FAM162B | family with sequence similarity | 27.3855 | 4.77534 | 4.42E−06 |
| | 162_member B | | | |
| ASXL3 | additional sex combs like transcriptional | 27.0169 | 4.75579 | 9.59E−06 |
| | regulator 3 | | | |
| EBI3 | Epstein-Barr virus induced 3 | 26.9236 | 4.7508 | 8.48E−11 |
| LYPLAL1-AS1 | LYPLAL1 antisense RNA 1 (head to head) | 26.8861 | 4.74879 | 2.75E−22 |
| ANKRD18B | ankyrin repeat domain 18B | 26.6734 | 4.73733 | 4.75E−11 |
| LLGL2 | lethal giant larvae homolog 2 (Drosophila) | 26.6686 | 4.73707 | 5.18E−26 |
| SRSF12 | serine/arginine-rich splicing factor 12 | 26.1794 | 4.71036 | 1.89E−31 |
| DLK1 | delta-like 1 homolog (Drosophila) | 26.1428 | 4.70834 | 1.22E−08 |
| TMPRSS11B | transmembrane protease_serine 11B | 26.0476 | 4.70308 | 1.68E−05 |
| IGF2BP3 | insulin-like growth factor 2 mRNA binding | 26.0092 | 4.70095 | 1.08E−69 |
| | protein 3 | | | |
| F11R | F11 receptor | 25.9993 | 4.7004 | 2.83E−29 |
| TNNI1 | troponin I type 1 (skeletal_slow) | 25.984 | 4.69955 | 1.41E−06 |
| MAGEB17 | melanoma antigen family B17 | 25.5824 | 4.67708 | 4.62E−06 |
| PPARG | peroxisome proliferator-activated | 25.1998 | 4.65534 | 1.06E−11 |
| | receptor gamma | | | |
| PLCB2 | phospholipase C_beta 2 | 25.1225 | 4.65091 | 8.52E−26 |
| HRASLS | HRAS-like suppressor | 25.1096 | 4.65017 | 3.45E−05 |
| JPH1 | junctophilin 1 | 25.0058 | 4.64419 | 3.39E−06 |
| EPHA7 | EPH receptor A7 | 24.8508 | 4.63522 | 3.06E−05 |
| PCYT1B | phosphate cytidylyltransferase | 24.7382 | 4.62867 | 4.96E−06 |
| | 1_choline_beta | | | |
| KIAA1211 | KIAA1211 | 24.6733 | 4.62488 | 5.54E−17 |
| ARL14 | ADP-ribosylation factor-like 14 | 24.6274 | 4.62219 | 5.67E−05 |
| VIP | vasoactive intestinal peptide | 24.5153 | 4.61561 | 1.86E−06 |
| LHX2 | LIM homeobox 2 | 24.445 | 4.61147 | 2.21E−08 |
| C4BPB | complement component 4 binding | 24.4286 | 4.6105 | 1.18E−07 |
| | protein_beta | | | |
| RSPO4 | R-spondin 4 | 24.3298 | 4.60465 | 2.80E−14 |
| YBX2 | Y box binding protein 2 | 24.2681 | 4.60099 | 1.18E−07 |
| THSD7A | thrombospondin_type I_domain containing | 24.2192 | 4.59808 | 2.09E−50 |
| | 7A | | | |
| SDK2 | sidekick cell adhesion molecule 2 | 24.0776 | 4.58962 | 1.10E−06 |
| HS6ST2 | heparan sulfate 6-O-sulfotransferase 2 | 23.8949 | 4.57863 | 3.22E−06 |
| PCDHB2 | protocadherin beta 2 | 23.8823 | 4.57787 | 1.41E−32 |
| PCDH10 | protocadherin 10 | 23.6912 | 4.56628 | 1.50E−07 |
| ICOSLG | inducible T-cell co-stimulator ligand | 23.6241 | 4.56219 | 5.57E−19 |
| IGF2BP1 | insulin-like growth factor 2 mRNA binding | 23.6132 | 4.56152 | 4.59E−76 |
| | protein 1 | | | |
| KCNF1 | potassium channel_voltage gated modifier | 23.6097 | 4.56131 | 4.19E−11 |
| | subfamily F_member 1 | | | |
| GDF7 | growth differentiation factor 7 | 23.5935 | 4.56032 | 9.44E−06 |
| EFNA2 | ephrin-A2 | 23.46 | 4.55213 | 3.31E−12 |
| CXADR | coxsackie virus and adenovirus receptor | 23.1897 | 4.53541 | 1.38E−08 |
| GLB1L2 | galactosidase_beta 1-like 2 | 23.1096 | 4.53042 | 4.28E−14 |
| IGFBP5 | insulin-like growth factor binding protein 5 | 22.9538 | 4.52066 | 8.23E−28 |
| KRT79 | keratin 79_type II | 22.9042 | 4.51754 | 5.91E−08 |
| IL33 | interleukin 33 | 22.8265 | 4.51264 | 5.10E−05 |
| CPA6 | carboxypeptidase A6 | 22.6992 | 4.50457 | 1.90E−05 |
| RGS1 | regulator of G-protein signaling 1 | 22.6241 | 4.49979 | 0.000116 |
| GPR63 | G protein-coupled receptor 63 | 22.6204 | 4.49955 | 4.35E−10 |
| DOC2GP | double C2-like | 22.592 | 4.49774 | 1.08E−10 |
| | domains_gamma_pseudogene | | | |
| FAM110D | family with sequence similarity | 22.2782 | 4.47756 | 2.13E−08 |
| | 110_member D | | | |
| ART5 | ADP-ribosyltransferase 5 | 22.0036 | 4.45967 | 6.14E−06 |
| CD163L1 | CD163 molecule-like 1 | 21.9861 | 4.45852 | 6.41E−25 |
| ATCAY | ataxia_cerebellar_Cayman type | 21.9716 | 4.45757 | 1.81E−05 |
| CNTN5 | contactin 5 | 21.7124 | 4.44045 | 0.000119 |
| LONRF2 | LON peptidase N-terminal domain and ring | 21.3584 | 4.41673 | 3.34E−16 |
| | finger 2 | | | |
| AFAP1L2 | actin filament associated protein 1-like 2 | 21.2551 | 4.40974 | 1.28E−12 |
| LRP1B | low density lipoprotein receptor-related | 21.1326 | 4.4014 | 0.000136 |
| | protein 1B | | | |
| HOXA13 | homeobox A13 | 21.1285 | 4.40112 | 1.71E−11 |
| LCP1 | lymphocyte cytosolic protein 1 (L-plastin) | 21.0927 | 4.39867 | 1.38E−11 |
| TNFSF4 | tumor necrosis factor (ligand) | 21.0743 | 4.39741 | 4.65E−65 |
| | superfamily_member 4 | | | |
| AQP7P3 | aquaporin 7 pseudogene 3 | 21.0438 | 4.39532 | 0.000248 |
| METTL24 | methyltransferase like 24 | 20.8515 | 4.38208 | 1.27E−05 |
| SULT4A1 | sulfotransferase family 4A_member 1 | 20.8356 | 4.38098 | 3.22E−20 |
| PDE6B | phosphodiesterase | 20.8092 | 4.37915 | 5.14E−22 |
| | 6B_cGMP-specific_rod_beta | | | |
| AQP7P1 | aquaporin 7 pseudogene 1 | 20.7323 | 4.37381 | 9.27E−07 |
| GUCY1A3 | guanylate cyclase 1_soluble_alpha 3 | 20.6716 | 4.36958 | 3.47E−05 |
| PCAT1 | prostate cancer associated transcript 1 | 20.6577 | 4.36861 | 0.000117 |
| | (non-protein coding) | | | |
| OTOS | otospiralin | 20.6125 | 4.36545 | 3.94E−07 |
| AQP5 | aquaporin 5 | 20.6021 | 4.36472 | 5.95E−07 |
| HES4 | hes family bHLH transcription factor 4 | 20.5841 | 4.36346 | 5.75E−14 |
| ADAMTS3 | ADAM metallopeptidase with | 20.527 | 4.35945 | 3.83E−34 |
| | thrombospondin type 1 motif_3 | | | |
| C1orf94 | chromosome 1 open reading frame 94 | 20.4524 | 4.3542 | 3.40E−05 |
| LOC101928303 | uncharacterized LOC101928303 | 20.4496 | 4.354 | 1.54E−05 |
| MOB3B | MOB kinase activator 3B | 20.3551 | 4.34732 | 1.05E−12 |
| ITIH3 | inter-alpha-trypsin inhibitor heavy chain 3 | 20.3247 | 4.34516 | 1.58E−13 |
| SUCNR1 | succinate receptor 1 | 20.1055 | 4.32952 | 0.000611 |
| ST8SIA2 | ST8 alpha-N-acetyl-neuraminide alpha-2_8- | 19.9714 | 4.31986 | 1.02E−05 |
| | sialyltransferase 2 | | | |
| PCDHA11 | protocadherin alpha 11 | 19.8527 | 4.31126 | 1.11E−07 |
| S1PR5 | sphingosine-1-phosphate receptor 5 | 19.7582 | 4.30438 | 5.60E−39 |
| LRRC4C | leucine rich repeat containing 4C | 19.7054 | 4.30052 | 3.88E−23 |
| GPRIN2 | G protein regulated inducer of neurite | 19.63 | 4.29499 | 1.04E−07 |
| | outgrowth 2 | | | |
| ANXA3 | annexin A3 | 19.3198 | 4.27201 | 6.49E−38 |
| UCP2 | uncoupling protein 2 (mitochondrial_proton | 19.1933 | 4.26253 | 9.86E−33 |
| | carrier) | | | |
| PRAC2 | prostate cancer susceptibility candidate 2 | 18.9331 | 4.24284 | 0.000291 |
| MAP3K9 | mitogen-activated protein kinase kinase | 18.9294 | 4.24256 | 2.16E−25 |
| | kinase 9 | | | |
| MYH14 | myosin_heavy chain 14_non-muscle | 18.9226 | 4.24204 | 3.05E−09 |
| SLITRK5 | SLIT and NTRK-like family_member 5 | 18.887 | 4.23932 | 0.000287 |
| RAMP2-AS1 | RAMP2 antisense RNA 1 | 18.881 | 4.23886 | 2.61E−14 |
| FRAS1 | Fraser extracellular matrix complex | 18.7633 | 4.22984 | 7.69E−22 |
| | subunit 1 | | | |
| DCHS1 | dachsous cadherin-related 1 | 18.7224 | 4.22669 | 1.91E−85 |
| PCBP3 | poly(rC) binding protein 3 | 18.6032 | 4.21748 | 5.50E−08 |
| DENND2A | DENN/MADD domain containing 2A | 18.5959 | 4.21691 | 2.08E−28 |
| CYTH4 | cytohesin 4 | 18.4855 | 4.20832 | 2.05E−05 |
| SYT3 | synaptotagmin III | 18.4219 | 4.20335 | 4.79E−10 |
| BEGAIN | brain-enriched guanylate kinase-associated | 18.3092 | 4.1945 | 5.24E−14 |
| SYT13 | synaptotagmin XIII | 18.3031 | 4.19402 | 1.84E−07 |
| PRKCQ | protein kinase C_theta | 18.3006 | 4.19382 | 1.34E−08 |
| ALPK3 | alpha-kinase 3 | 18.0415 | 4.17325 | 8.05E−43 |
| INPP5D | inositol polyphosphate-5-phosphatase D | 18.0215 | 4.17165 | 1.99E−10 |
| CLEC14A | C-type lectin domain family 14_member A | 17.9653 | 4.16714 | 2.07E−10 |
| GRAP | GRB2-related adaptor protein | 17.9383 | 4.16497 | 9.76E−14 |
| MYCT1 | myc target 1 | 17.8007 | 4.15386 | 5.96E−17 |
| SPINT1 | serine peptidase inhibitor_Kunitz type 1 | 17.6879 | 4.14469 | 1.47E−10 |
| LINC00951 | long intergenic non-protein coding RNA 951 | 17.6694 | 4.14318 | 0.000391 |
| SLC1A7 | solute carrier family 1 (glutamate | 17.6269 | 4.13971 | 5.10E−21 |
| | transporter)_member 7 | | | |
| PLN | phospholamban | 17.6175 | 4.13894 | 2.23E−05 |
| CDH8 | cadherin 8_type 2 | 17.6042 | 4.13785 | 5.23E−06 |
| SCN2A | sodium channel_voltage gated_type II | 17.5982 | 4.13736 | 1.84E−07 |
| | alpha subunit | | | |
| OR2H2 | olfactory receptor_family 2_subfamily | 17.4823 | 4.12782 | 5.83E−06 |
| | H_member 2 | | | |
| TNNI3 | troponin I type 3 (cardiac) | 17.2932 | 4.11213 | 8.76E−06 |
| SNCB | synuclein_beta | 17.2161 | 4.10569 | 2.97E−05 |
| PRSS16 | protease_serine_16 (thymus) | 17.1239 | 4.09794 | 1.53E−05 |
| NNAT | neuronatin | 17.1189 | 4.09752 | 9.80E−78 |
| ZBTB46 | zinc finger and BTB domain containing 46 | 17.1106 | 4.09682 | 1.95E−29 |
| SLC6A12 | solute carrier family 6 (neurotransmitter | 17.0269 | 4.08974 | 0.000365 |
| | transporter)_member 12 | | | |
| EPB41L3 | erythrocyte membrane protein band 4.1- | 17.0146 | 4.0887 | 0.001336 |
| | like 3 | | | |
| IL1A | interleukin 1_alpha | 16.9791 | 4.08569 | 6.19E−09 |
| GRIN2A | glutamate receptor_ionotropic_N-methyl | 16.9785 | 4.08564 | 2.15E−06 |
| | D-aspartate 2A | | | |
| HBE1 | hemoglobin_epsilon 1 | 16.9763 | 4.08545 | 3.06E−05 |
| LIPH | lipase_member H | 16.9375 | 4.08215 | 0.000383 |
| EMCN | endomucin | 16.9336 | 4.08182 | 1.72E−05 |
| NTRK3 | neurotrophic tyrosine kinase_receptor_{—} | 16.9244 | 4.08103 | 4.31E−17 |
| | type 3 | | | |
| TMEFF2 | transmembrane protein with EGF-like and | 16.918 | 4.08049 | 3.89E−06 |
| | two follistatin-like domains 2 | | | |
| N4BP3 | NEDD4 binding protein 3 | 16.7396 | 4.06519 | 5.46E−10 |
| LINC00460 | long intergenic non-protein coding RNA 460 | 16.6098 | 4.05396 | 1.41E−09 |
| SCARF1 | scavenger receptor class F_member 1 | 16.605 | 4.05355 | 2.79E−51 |
| SMCO3 | single-pass membrane protein with coiled- | 16.529 | 4.04693 | 1.48E−13 |
| | coil domains 3 | | | |
| FBXL16 | F-box and leucine-rich repeat protein 16 | 16.4858 | 4.04315 | 1.21E−14 |
| SLC16A12 | solute carrier family 16_member 12 | 16.4363 | 4.03881 | 2.11E−10 |
| IRX4 | iroquois homeobox 4 | 16.3644 | 4.03249 | 0.00015 |
| F2RL1 | coagulation factor II (thrombin) receptor- | 16.3467 | 4.03093 | 1.05E−12 |
| | like 1 | | | |
| PLCH2 | phospholipase C_eta 2 | 16.2672 | 4.02389 | 1.91E−20 |
| EPCAM | epithelial cell adhesion molecule | 16.2263 | 4.02026 | 8.21E−19 |
| TNFRSF9 | tumor necrosis factor receptor | 16.1981 | 4.01775 | 4.31E−20 |
| | superfamily_member 9 | | | |
| CCDC3 | coiled-coil domain containing 3 | 16.1881 | 4.01686 | 2.55E−20 |
| SOX8 | SRY (sex determining region Y)-box 8 | 16.1306 | 4.01173 | 3.37E−09 |
| PTPN6 | protein tyrosine phosphatase_non-receptor | 16.1207 | 4.01084 | 2.13E−21 |
| | type 6 | | | |
| PDGFRL | platelet-derived growth factor receptor-like | 16.015 | 4.00135 | 1.75E−28 |
| CBLN2 | cerebellin 2 precursor | 15.984 | 3.99856 | 0.001124 |
| NLRP2 | NLR family_pyrin domain containing 2 | 15.9836 | 3.99852 | 2.37E−11 |
| EXPH5 | exophilin 5 | 15.9414 | 3.99471 | 3.32E−10 |
| CNTN1 | contactin 1 | 15.9247 | 3.99319 | 9.80E−09 |
| ACHE | acetylcholinesterase (Yt blood group) | 15.8565 | 3.987 | 2.21E−18 |
| GPR112 | adhesion G protein-coupled receptor G4 | 15.7991 | 3.98177 | 5.31E−06 |
| FAM84B | family with sequence similarity | 15.7964 | 3.98152 | 6.39E−95 |
| | 84_member B | | | |
| PARM1 | prostate androgen-regulated mucin-like | 15.771 | 3.9792 | 2.10E−09 |
| | protein 1 | | | |
| B3GNT5 | UDP-GlcNAc:betaGal beta-1_3-N- | 15.7637 | 3.97853 | 2.55E−29 |
| | acetylglucosaminyltransferase 5 | | | |
| MCF2L | MCF.2 cell line derived transforming | 15.7588 | 3.97809 | 6.13E−05 |
| | sequence-like | | | |
| F10 | coagulation factor X | 15.7575 | 3.97797 | 3.70E−17 |
| RAB26 | RAB26_member RAS oncogene family | 15.7496 | 3.97724 | 1.59E−23 |
| OR51E2 | olfactory receptor_family 51_subfamily | 15.7274 | 3.97521 | 0.000128 |
| | E_member 2 | | | |
| ANXA13 | annexin A13 | 15.6535 | 3.96841 | 0.000199 |
| SLC12A5 | solute carrier family 12 (potassium/chloride | 15.6414 | 3.9673 | 3.07E−10 |
| | transporter)_member 5 | | | |
| ARHGEF26 | Rho guanine nucleotide exchange factor | 15.637 | 3.96689 | 6.21E−18 |
| | (GEF) 26 | | | |
| CLDN1 | claudin 1 | 15.6055 | 3.96398 | 8.58E−16 |
| HMGA2 | high mobility group AT-hook 2 | 15.5802 | 3.96164 | 3.74E−38 |
| SYT9 | synaptotagmin IX | 15.5294 | 3.95693 | 0.000275 |
| COX6B2 | cytochrome c oxidase subunit VIb | 15.4751 | 3.95188 | 3.39E−15 |
| | polypeptide 2 (testis) | | | |
| SLC9A4 | solute carrier family 9_subfamily A | 15.4171 | 3.94646 | 0.00011 |
| | (NHE4_cation proton antiporter | | | |
| | 4)_member 4 | | | |
| SLITRK6 | SLIT and NTRK-like family_member 6 | 15.4144 | 3.94621 | 0.001369 |
| DOCK8 | dedicator of cytokinesis 8 | 15.414 | 3.94617 | 0.000138 |
| GPR126 | adhesion G protein-coupled receptor G6 | 15.3514 | 3.9403 | 5.60E−39 |
| LOC100130238 | uncharacterized LOC100130238 | 15.2641 | 3.93207 | 2.52E−05 |
| SULT1C2 | sulfotransferase | 15.2025 | 3.92624 | 4.89E−05 |
| | family_cytosolic_1C_member 2 | | | |
| NIPAL1 | NIPA-like domain containing 1 | 15.1915 | 3.92519 | 9.10E−12 |
| GNA14 | guanine nucleotide binding protein (G | 15.1356 | 3.91987 | 5.65E−26 |
| | protein)_alpha 14 | | | |
| PRKCQ-AS1 | PRKCQ antisense RNA 1 | 15.0898 | 3.9155 | 2.20E−14 |
| LOC102800447 | uncharacterized LOC102800447 | 15.0873 | 3.91526 | 9.37E−06 |
| KCNS1 | potassium voltage-gated channel_modifier | 15.0747 | 3.91406 | 3.97E−16 |
| | subfamily S_member 1 | | | |
| LOC100126784 | uncharacterized LOC100126784 | 15.0471 | 3.91141 | 1.38E−33 |
| LPHN3 | adhesion G protein-coupled receptor L3 | 14.9638 | 3.9034 | 0.000136 |
| TMIGD2 | transmembrane and immunoglobulin domain | 14.9566 | 3.90271 | 0.001397 |
| | containing 2 | | | |
| VSTM1 | V-set and transmembrane domain containing | 14.8319 | 3.89063 | 0.001267 |
| | 1 | | | |
| CDH3 | cadherin 3_type 1_P-cadherin (placental) | 14.8271 | 3.89016 | 2.48E−20 |
| PRKCZ | protein kinase C_zeta | 14.7712 | 3.88472 | 2.05E−20 |
| MAP2 | microtubule-associated protein 2 | 14.7558 | 3.88321 | 4.24E−17 |
| PIK3AP1 | phosphoinositide-3-kinase adaptor protein 1 | 14.7409 | 3.88175 | 5.73E−06 |
| TNFSF18 | tumor necrosis factor (ligand) | 14.7276 | 3.88045 | 0.001118 |
| | superfamily_member 18 | | | |
| MIR4697HG | MIR4697 host gene | 14.6591 | 3.87372 | 3.42E−07 |
| GP6 | glycoprotein VI (platelet) | 14.6537 | 3.87319 | 0.000236 |
| LINC01021 | long intergenic non-protein coding RNA | 14.6366 | 3.87151 | 2.94E−08 |
| | 1021 | | | |
| PLAC8 | placenta-specific 8 | 14.5941 | 3.86731 | 2.84E−39 |
| TMEM88 | transmembrane protein 88 | 14.5881 | 3.86672 | 9.16E−19 |
| ENTPD8 | ectonucleoside triphosphate | 14.5833 | 3.86625 | 0.000548 |
| | diphosphohydrolase 8 | | | |
| PPARGC1A | peroxisome proliferator-activated receptor | 14.5566 | 3.8636 | 9.48E−07 |
| | gamma_coactivator 1 alpha | | | |
| SH3GL2 | SH3-domain GRB2-like 2 | 14.4701 | 3.855 | 1.65E−06 |
| SCN9A | sodium channel_voltage gated_type IX | 14.4341 | 3.85141 | 9.10E−23 |
| | alpha subunit | | | |
| CPNE7 | copine VII | 14.4104 | 3.84904 | 1.53E−18 |
| NRARP | NOTCH-regulated ankyrin repeat protein | 14.4019 | 3.84819 | 6.33E−13 |
| CERS4 | ceramide synthase 4 | 14.3768 | 3.84567 | 1.43E−21 |
| FCHO1 | FCH domain only 1 | 14.3756 | 3.84555 | 1.82E−20 |
| C19orf81 | chromosome 19 open reading frame 81 | 14.3156 | 3.83952 | 4.03E−09 |
| PGM5 | phosphoglucomutase 5 | 14.3137 | 3.83932 | 5.56E−07 |
| LINC01082 | long intergenic non-protein coding RNA | 14.2988 | 3.83782 | 0.002369 |
| | 1082 | | | |
| HIST1H2BG | histone cluster 1_H2bg | 14.2247 | 3.83033 | 5.47E−11 |
| LOC100507006 | uncharacterized LOC100507006 | 14.1543 | 3.82317 | 0.002106 |
| LMTK3 | lemur tyrosine kinase 3 | 14.1398 | 3.82169 | 2.65E−37 |
| QPRT | quinolinate phosphoribosyltransferase | 14.1045 | 3.81808 | 6.47E−60 |
| TMEM35 | transmembrane protein 35 | 14.0929 | 3.8169 | 7.46E−19 |
| SEMA6B | sema domain_transmembrane domain | 14.0663 | 3.81417 | 1.78E−13 |
| | (TM)_and cytoplasmic | | | |
| | domain_(semaphorin) 6B | | | |
| AADACP1 | arylacetamide deacetylase pseudogene 1 | 14.0634 | 3.81387 | 9.06E−05 |
| CDH5 | cadherin 5_type 2 (vascular endothelium) | 14.0396 | 3.81143 | 3.35E−05 |
| ZNF521 | zinc finger protein 521 | 13.9425 | 3.80142 | 3.49E−07 |
| ZYG11A | zyg-11 family member A_cell cycle | 13.8672 | 3.7936 | 3.38E−14 |
| | regulator | | | |
| LINC00880 | long intergenic non-protein coding RNA 880 | 13.826 | 3.78931 | 3.87E−06 |
| DENND1C | DENN/MADD domain containing 1C | 13.8021 | 3.78682 | 9.80E−07 |
| LOC101927746 | uncharacterized LOC101927746 | 13.6537 | 3.77122 | 1.42E−10 |
| TRPV6 | transient receptor potential cation | 13.6524 | 3.77108 | 0.000789 |
| | channel_subfamily V_member 6 | | | |
| CAMK1G | calcium/calmodulin-dependent protein | 13.5132 | 3.7563 | 8.50E−09 |
| | kinase IG | | | |
| ELOVL2-AS1 | ELOVL2 antisense RNA 1 | 13.4665 | 3.7513 | 0.000246 |
| CYFIP2 | cytoplasmic FMR1 interacting protein 2 | 13.3884 | 3.74291 | 8.57E−94 |
| NOS1AP | nitric oxide synthase 1 (neuronal) adaptor | 13.3866 | 3.74272 | 3.46E−07 |
| | protein | | | |
| TRHDE | thyrotropin-releasing hormone degrading | 13.2802 | 3.73121 | 1.32E−13 |
| | enzyme | | | |
| LSAMP-AS1 | LSAMP antisense RNA 1 | 13.2538 | 3.72833 | 0.000305 |
| SPOCK3 | sparc/osteonectin_cwcv and kazal-like | 13.2293 | 3.72566 | 0.0022 |
| | domains proteoglycan (testican) 3 | | | |
| MPZL2 | myelin protein zero-like 2 | 13.2262 | 3.72533 | 0.001202 |
| LAMA5 | laminin_alpha 5 | 13.2086 | 3.72341 | 1.87E−24 |
| LOC101929690 | NA | 13.2059 | 3.72311 | 1.65E−16 |
| F7 | coagulation factor VII (serum prothrombin | 13.1548 | 3.71752 | 0.001195 |
| | conversion accelerator) | | | |
| LOC101927482 | uncharacterized LOC101927482 | 13.1233 | 3.71406 | 3.80E−09 |
| ACSM4 | acyl-CoA synthetase medium-chain family | 13.0723 | 3.70844 | 0.00141 |
| | member 4 | | | |
| KLHL6 | kelch-like family member 6 | 13.0632 | 3.70744 | 0.000927 |
| MUC22 | mucin 22 | 13.0296 | 3.70372 | 0.001228 |
| FGF13 | fibroblast growth factor 13 | 13.018 | 3.70244 | 0.004351 |
| F3 | coagulation factor III | 12.9766 | 3.69784 | 1.07E−10 |
| | (thromboplastin_tissue factor) | | | |
| TMSB15A | thymosin beta 15a | 12.9458 | 3.69441 | 1.02E−14 |
| KSR1 | kinase suppressor of ras 1 | 12.9175 | 3.69125 | 1.82E−74 |
| CERS1 | ceramide synthase 1 | 12.9124 | 3.69068 | 5.47E−10 |
| TNIK | TRAF2 and NCK interacting kinase | 12.8675 | 3.68566 | 2.39E−15 |
| PKIB | protein kinase (cAMP-dependent_catalytic) | 12.7595 | 3.6735 | 9.98E−05 |
| | inhibitor beta | | | |
| C1orf226 | chromosome 1 open reading frame 226 | 12.7485 | 3.67225 | 1.48E−05 |
| DEF6 | DEF6 guanine nucleotide exchange factor | 12.7046 | 3.66728 | 3.26E−28 |
| RCVRN | recoverin | 12.6679 | 3.6631 | 0.001865 |
| IL31RA | interleukin 31 receptor A | 12.6668 | 3.66298 | 1.03E−08 |
| SOWAHB | sosondowah ankyrin repeat domain family | 12.6359 | 3.65946 | 5.40E−07 |
| | member B | | | |
| MIR2682 | microRNA 2682 | 12.6238 | 3.65808 | 2.24E−37 |
| SH2D5 | SH2 domain containing 5 | 12.6064 | 3.65608 | 1.12E−31 |
| ST6GALNAC5 | ST6 (alpha-N-acetyl-neuraminyl-2_3-beta- | 12.5647 | 3.65131 | 6.63E−05 |
| | galactosyl-1_3)-N-acetylgalactosaminide | | | |
| | alpha-2_6-sialyltransferase 5 | | | |
| TNFRSF10C | tumor necrosis factor receptor superfamily | 12.5125 | 3.6453 | 1.63E−21 |
| | member 10c_decoy without an intracellular | | | |
| | domain | | | |
| GJA3 | gap junction protein_alpha 3_46kDa | 12.427 | 3.63541 | 2.24E−05 |
| ELAVL2 | ELAV like neuron-specific RNA binding | 12.3924 | 3.63138 | 2.84E−06 |
| | protein 2 | | | |
| ERC2 | ELKS/RAB6-interacting/CAST family | 12.358 | 3.62737 | 3.63E−05 |
| | member 2 | | | |
| CAPN11 | calpain 11 | 12.356 | 3.62714 | 0.0006 |
| C7orf69 | chromosome 7 open reading frame 69 | 12.3522 | 3.6267 | 1.28E−17 |
| KIF17 | kinesin family member 17 | 12.3375 | 3.62498 | 1.04E−21 |
| ZBED2 | zinc finger_BED-type containing 2 | 12.2674 | 3.61676 | 1.51E−06 |
| TTYH2 | tweety family member 2 | 12.2659 | 3.61658 | 1.79E−86 |
| ST18 | suppression of tumorigenicity 18_zinc | 12.2605 | 3.61595 | 0.000105 |
| | finger | | | |
| GRB14 | growth factor receptor-bound protein 14 | 12.2588 | 3.61574 | 2.43E−31 |
| EDN2 | endothelin 2 | 12.2511 | 3.61484 | 2.03E−06 |
| KCP | kielin/chordin-like protein | 12.2016 | 3.609 | 4.14E−05 |
| MESTIT1 | MEST intronic transcript 1_antisense RNA | 12.1456 | 3.60236 | 0.000424 |
| CLGN | calmegin | 12.1193 | 3.59923 | 2.73E−09 |
| IL18 | interleukin 18 | 12.101 | 3.59706 | 6.08E−15 |
| ANKRD18A | ankyrin repeat domain 18A | 12.0977 | 3.59666 | 1.23E−05 |
| UPB1 | ureidopropionase_beta | 12.0815 | 3.59473 | 0.000346 |
| CARD11 | caspase recruitment domain | 12.066 | 3.59287 | 4.03E−13 |
| | family_member 11 | | | |
| KLHL23 | kelch-like family member 23 | 12.0564 | 3.59173 | 0.003407 |
| ABCD2 | ATP-binding cassette_sub-family D | 12.0107 | 3.58625 | 0.000115 |
| | (ALD)_member 2 | | | |
| ITGAX | integrin_alpha X (complement component 3 | 11.9821 | 3.58281 | 0.003088 |
| | receptor 4 subunit) | | | |
| CDH18 | cadherin 18_type 2 | 11.9667 | 3.58095 | 0.004769 |
| NOX4 | NADPH oxidase 4 | 11.9252 | 3.57594 | 7.16E−28 |
| TMEM125 | transmembrane protein 125 | 11.9135 | 3.57452 | 0.000735 |
| PPARGC1B | peroxisome proliferator-activated receptor | 11.8045 | 3.56127 | 4.66E−06 |
| | gamma_coactivator 1 beta | | | |
| F2 | coagulation factor II (thrombin) | 11.7803 | 3.5583 | 9.71E−05 |
| CAMSAP3 | calmodulin regulated spectrin-associated | 11.777 | 3.5579 | 0.001984 |
| | protein family_member 3 | | | |
| LOC100996579 | uncharacterized LOC100996579 | 11.7569 | 3.55544 | 0.000352 |
| FBXO2 | F-box protein 2 | 11.7289 | 3.55199 | 2.38E−28 |
| ZNF663P | zinc finger protein 663_pseudogene | 11.713 | 3.55004 | 1.20E−05 |
| KCNK3 | potassium channel_two pore domain | 11.7019 | 3.54867 | 9.68E−15 |
| | subfamily K_member 3 | | | |
| OGDHL | oxoglutarate dehydrogenase-like | 11.6171 | 3.53818 | 1.44E−11 |
| HTR1B | 5-hydroxytryptamine (serotonin) receptor | 11.5855 | 3.53425 | 0.006255 |
| | 1B_G protein-coupled | | | |
| NPW | neuropeptide W | 11.5805 | 3.53363 | 8.71E−28 |
| RND2 | Rho family GTPase 2 | 11.5602 | 3.53109 | 2.29E−19 |
| POU2F3 | POU class 2 homeobox 3 | 11.4708 | 3.51989 | 0.000573 |
| BAIAP3 | BAI1-associated protein 3 | 11.4524 | 3.51758 | 4.89E−11 |
| PCDHA9 | protocadherin alpha 9 | 11.4289 | 3.51461 | 0.00541 |
| INA | internexin neuronal intermediate filament | 11.406 | 3.51172 | 1.76E−19 |
| | protein_alpha | | | |
| LINC01012 | long intergenic non-protein coding RNA | 11.3526 | 3.50495 | 0.000134 |
| | 1012 | | | |
| FLT4 | fms-related tyrosine kinase 4 | 11.3474 | 3.50429 | 9.13E−06 |
| FAR2P2 | fatty acyl CoA reductase 2 pseudogene 2 | 11.298 | 3.49799 | 1.10E−05 |
| PALM3 | paralemmin 3 | 11.2908 | 3.49708 | 3.25E−22 |
| LINC00887 | long intergenic non-protein coding RNA 887 | 11.2675 | 3.4941 | 9.65E−05 |
| HSD17B14 | hydroxysteroid (17-beta) dehydrogenase 14 | 11.261 | 3.49326 | 8.14E−10 |
| ZNF853 | zinc finger protein 853 | 11.2476 | 3.49155 | 5.44E−17 |
| TYROBP | TYRO protein tyrosine kinase binding | 11.2225 | 3.48832 | 0.004002 |
| | protein | | | |
| FCGBP | Fc fragment of IgG binding protein | 11.193 | 3.48453 | 2.53E−08 |
| LOC349160 | uncharacterized LOC349160 | 11.1929 | 3.48451 | 0.00353 |
| C10orf91 | chromosome 10 open reading frame 91 | 11.1722 | 3.48184 | 1.42E−05 |
| PCDH9 | protocadherin 9 | 11.1083 | 3.47356 | 4.97E−14 |
| CD101 | CD101 molecule | 11.0942 | 3.47174 | 3.83E−07 |
| PCDHA4 | protocadherin alpha 4 | 11.0596 | 3.46723 | 7.10E−06 |
| LINC00858 | long intergenic non-protein coding RNA 858 | 11.0374 | 3.46433 | 0.007369 |
| SPACA4 | sperm acrosome associated 4 | 10.9188 | 3.44874 | 0.005785 |
| C14orf39 | chromosome 14 open reading frame 39 | 10.9121 | 3.44786 | 1.16E−15 |
| JUP | junction plakoglobin | 10.8918 | 3.44517 | 1.02E−41 |
| KIF21B | kinesin family member 21B | 10.847 | 3.43922 | 4.67E−31 |
| NPPB | natriuretic peptide B | 10.8154 | 3.43502 | 6.87E−06 |
| GALNTL6 | polypeptide N- | 10.803 | 3.43336 | 0.001067 |
| | acetylgalactosaminyltransferase-like 6 | | | |
| PCDHGB6 | protocadherin gamma subfamily B_6 | 10.7364 | 3.42444 | 5.77E−09 |
| KIAA1257 | KIAA1257 | 10.7286 | 3.42339 | 0.00706 |
| DNM1 | dynamin 1 | 10.7235 | 3.42271 | 6.95E−21 |
| CRB2 | crumbs family member 2 | 10.6856 | 3.4176 | 7.06E−05 |
| ECSCR | endothelial cell surface expressed | 10.64 | 3.41143 | 4.77E−18 |
| | chemotaxis and apoptosis regulator | | | |
| SRRM4 | serine/arginine repetitive matrix 4 | 10.595 | 3.40531 | 3.37E−08 |
| SLC27A2 | solute carrier family 27 (fatty acid | 10.5673 | 3.40153 | 8.96E−05 |
| | transporter)_member 2 | | | |
| ATRNL1 | attractin-like 1 | 10.5349 | 3.39711 | 1.52E−13 |
| PEG10 | paternally expressed 10 | 10.4808 | 3.38968 | 2.60E−13 |
| NFAM1 | NFAT activating protein with ITAM motif 1 | 10.3784 | 3.37551 | 0.00437 |
| BLACAT1 | bladder cancer associated transcript 1 | 10.3481 | 3.37129 | 0.000494 |
| | (non-protein coding) | | | |
| HSD17B2 | hydroxysteroid (17-beta) dehydrogenase 2 | 10.3443 | 3.37077 | 0.006844 |
| MEX3A | mex-3 RNA binding family member A | 10.2924 | 3.36351 | 0.00E+00 |
| LOC100129617 | uncharacterized LOC100129617 | 10.2675 | 3.36001 | 7.93E−06 |
| IGLON5 | IgLON family member 5 | 10.2593 | 3.35886 | 4.35E−05 |
| AQP1 | aquaporin 1 (Colton blood group) | 10.2493 | 3.35746 | 2.90E−52 |
| ERBB4 | erb-b2 receptor tyrosine kinase 4 | 10.2334 | 3.35521 | 0.003618 |
| MGAT5B | mannosyl (alpha-1_6-)-glycoprotein beta- | 10.2166 | 3.35284 | 1.50E−28 |
| | 1_6-N-acetyl- | | | |
| | glucosaminyltransferase_isozyme B | | | |
| EPHB6 | EPH receptor B6 | 10.2139 | 3.35246 | 6.39E−10 |
| CTAGE11P | CTAGE family_member 11_pseudogene | 10.2083 | 3.35167 | 0.000199 |
| HOXB-AS3 | HOXB cluster antisense RNA 3 | 10.1938 | 3.34962 | 6.77E−33 |
| LOC102723854 | uncharacterized LOC102723854 | 10.1809 | 3.3478 | 7.33E−06 |
| KCNN3 | potassium channel_calcium activated | 10.1753 | 3.347 | 7.11E−05 |
| | intermediate/small conductance subfamily N | | | |
| | alpha_member 3 | | | |
| DCDC2 | doublecortin domain containing 2 | 10.1005 | 3.33636 | 0.00503 |
| ZFP92 | ZFP92 zinc finger protein | 10.0974 | 3.33591 | 4.67E−05 |
| UPK1A-AS1 | UPK1A antisense RNA 1 | 10.0951 | 3.33558 | 0.004621 |
| HIST1H2BE | histone cluster 1_H2be | 10.0917 | 3.3351 | 6.66E−05 |
| RIMS2 | regulating synaptic membrane exocytosis 2 | 10.0667 | 3.33152 | 0.001381 |
| WSCD1 | WSC domain containing 1 | 10.0653 | 3.33132 | 0.000557 |
| LOC100507534 | uncharacterized LOC100507534 | 10.0514 | 3.32933 | 2.13E−05 |
| FSIP2 | fibrous sheath interacting protein 2 | 10.0377 | 3.32735 | 4.93E−09 |
| FGD4 | FYVE_RhoGEF and PH domain containing | 10.0126 | 3.32375 | 5.09E−57 |
| | 4 | | | |
| CTSC | cathepsin C | 10.0114 | 3.32357 | 3.86E−17 |
| RASL10A | RAS-like_family 10_member A | 10.0097 | 3.32333 | 3.46E−05 |
| JSRP1 | junctional sarcoplasmic reticulum protein 1 | 9.98879 | 3.32031 | 0.000419 |
| ERVMER34-1 | endogenous retrovirus group | 9.96811 | 3.31732 | 0.002564 |
| | MER34_member 1 | | | |
| ITGA2 | integrin_alpha 2 (CD49B_alpha 2 subunit | 9.96486 | 3.31685 | 3.74E−13 |
| | of VLA-2 receptor) | | | |
| LOC101927043 | uncharacterized LOC101927043 | 9.94692 | 3.31425 | 5.99E−05 |
| PROZ | protein Z_vitamin K-dependent plasma | 9.93142 | 3.312 | 9.36E−09 |
| | glycoprotein | | | |
| NR2F2-AS1 | NR2F2 antisense RNA 1 | 9.92254 | 3.31071 | 1.80E−45 |
| PLAC1 | placenta-specific 1 | 9.91313 | 3.30934 | 6.64E−08 |
| NMNAT3 | nicotinamide nucleotide adenylyltransferase | 9.90969 | 3.30884 | 1.04E−05 |
| | 3 | | | |
| TMEM51 | transmembrane protein 51 | 9.9007 | 3.30753 | 1.11E−35 |
| ZIC2 | Zic family member 2 | 9.89967 | 3.30738 | 0.006266 |
| LOC100507600 | uncharacterized LOC100507600 | 9.83537 | 3.29798 | 3.68E−35 |
| AR | androgen receptor | 9.7898 | 3.29128 | 3.71E−23 |
| ALOX15 | arachidonate 15-lipoxygenase | 9.71686 | 3.28049 | 0.006396 |
| ROR2 | receptor tyrosine kinase-like orphan | 9.70407 | 3.27859 | 9.56E−11 |
| | receptor 2 | | | |
| MBP | myelin basic protein | 9.66694 | 3.27306 | 5.54E−07 |
| LEMD1-AS1 | LEMD 1 antisense RNA 1 | 9.66353 | 3.27255 | 0.000186 |
| TMEM151B | transmembrane protein 151B | 9.66085 | 3.27215 | 0.000879 |
| EGLN3 | egl-9 family hypoxia-inducible factor 3 | 9.65482 | 3.27125 | 5.65E−06 |
| SGIP1 | SH3-domain GRB2-like (endophilin) | 9.64934 | 3.27043 | 1.12E−11 |
| | interacting protein 1 | | | |
| OVCH2 | ovochymase 2 (gene/pseudogene) | 9.61196 | 3.26483 | 4.28E−22 |
| PRKAR2B | protein kinase_cAMP- | 9.57233 | 3.25887 | 1.50E−16 |
| | dependent_regulatory_type II_beta | | | |
| PURG | purine-rich element binding protein G | 9.549 | 3.25535 | 1.64E−07 |
| KRT19 | keratin 19_type I | 9.51392 | 3.25004 | 3.57E−18 |
| NFE2L3 | nuclear factor_erythroid 2-like 3 | 9.51267 | 3.24985 | 7.63E−18 |
| FILIP1 | filamin A interacting protein 1 | 9.49936 | 3.24783 | 1.05E−06 |
| MYOCD | myocardin | 9.48028 | 3.24493 | 3.68E−49 |
| KCNQ1 | potassium channel_voltage gated KQT-like | 9.44205 | 3.2391 | 0.003517 |
| | subfamily Q_member 1 | | | |
| ACTBL2 | actin_beta-like 2 | 9.43662 | 3.23827 | 3.82E−08 |
| NUP62CL | nucleoporin 62kDa C-terminal like | 9.4291 | 3.23712 | 0.007505 |
| POTEF | POTE ankyrin domain family_member F | 9.42244 | 3.2361 | 7.58E−23 |
| FAM83E | family with sequence similarity | 9.41016 | 3.23422 | 0.000192 |
| | 83_member E | | | |
| CPA4 | carboxypeptidase A4 | 9.39003 | 3.23113 | 1.67E−160 |
| FAM183A | family with sequence similarity | 9.38463 | 3.2303 | 0.000142 |
| | 183_member A | | | |
| DUSP9 | dual specificity phosphatase 9 | 9.37663 | 3.22907 | 1.19E−05 |
| MYOM3 | myomesin 3 | 9.30657 | 3.21825 | 4.91E−08 |
| BCL11A | B-cell CLL/lymphoma 11A (zinc finger | 9.29523 | 3.21649 | 8.22E−07 |
| | protein) | | | |
| GEM | GTP binding protein overexpressed in | 9.27688 | 3.21364 | 4.53E−64 |
| | skeletal muscle | | | |
| TRABD2A | TraB domain containing 2A | 9.25383 | 3.21005 | 3.80E−23 |
| SPTBN2 | spectrin_beta_non-erythrocytic 2 | 9.25318 | 3.20995 | 5.85E−33 |
| ZP1 | zona pellucida glycoprotein 1 (sperm | 9.24959 | 3.20939 | 9.78E−05 |
| | receptor) | | | |
| VTRNA1-3 | vault RNA 1-3 | 9.24312 | 3.20838 | 0.001629 |
| FNDC9 | fibronectin type III domain containing 9 | 9.23524 | 3.20715 | 0.004547 |
| PPAP2C | phosphatidic acid phosphatase type 2C | 9.21574 | 3.2041 | 1.87E−06 |
| SERPINB7 | serpin peptidase inhibitor_clade B | 9.21146 | 3.20343 | 1.69E−16 |
| | (ovalbumin)_member 7 | | | |
| LOC645752 | golgin A6 family_member A pseudogene | 9.19169 | 3.20033 | 0.0005 |
| SLC4A9 | solute carrier family 4_sodium bicarbonate | 9.19029 | 3.20011 | 0.000273 |
| | cotransporter_member 9 | | | |
| SLC37A1 | solute carrier family 37 (glucose-6- | 9.18838 | 3.19981 | 3.01E−24 |
| | phosphate transporter)_member 1 | | | |
| FSTL4 | follistatin-like 4 | 9.12858 | 3.19039 | 0.009879 |
| NTF4 | neurotrophin 4 | 9.11094 | 3.1876 | 9.13E−10 |
| DRP2 | dystrophin related protein 2 | 9.0987 | 3.18566 | 6.82E−39 |
| ILDR2 | immunoglobulin-like domain containing | 9.06646 | 3.18054 | 0.001062 |
| | receptor 2 | | | |
| HBEGF | heparin-binding EGF-like growth factor | 9.04249 | 3.17672 | 3.30E−40 |
| MDFI | MyoD family inhibitor | 9.03672 | 3.1758 | 3.42E−14 |
| MED12L | mediator complex subunit 12-like | 9.0187 | 3.17292 | 3.30E−16 |
| TMCC3 | transmembrane and coiled-coil domain | 8.99436 | 3.16902 | 2.07E−08 |
| | family 3 | | | |
| FBXL13 | F-box and leucine-rich repeat protein 13 | 8.97206 | 3.16544 | 1.13E−16 |
| LOC100652824 | NA | 8.96603 | 3.16447 | 2.05E−07 |
| NSG1 | neuron specific gene family member 1 | 8.91305 | 3.15592 | 8.80E−18 |
| KRT18 | keratin 18_type I | 8.89023 | 3.15222 | 2.87E−11 |
| DOC2A | double C2-like domains_alpha | 8.88388 | 3.15119 | 1.98E−07 |
| LOC642366 | uncharacterized LOC642366 | 8.88142 | 3.15079 | 0.000859 |
| NOS3 | nitric oxide synthase 3 (endothelial cell) | 8.85572 | 3.14661 | 1.46E−09 |
| LPPR3 | lipid phosphate phosphatase-related protein | 8.84382 | 3.14467 | 9.86E−23 |
| | type 3 | | | |
| DACH2 | dachshund family transcription factor 2 | 8.83978 | 3.14401 | 0.008437 |
| C16orf74 | chromosome 16 open reading frame 74 | 8.82148 | 3.14102 | 8.22E−22 |
| CAMK4 | calcium/calmodulin-dependent protein | 8.81329 | 3.13968 | 6.34E−10 |
| | kinase IV | | | |
| EMID1 | EMI domain containing 1 | 8.80797 | 3.13881 | 8.00E−06 |
| SSPO | SCO-spondin | 8.79778 | 3.13714 | 1.54E−09 |
| ST6GAL1 | ST6 beta-galactosamide alpha-2_6- | 8.79705 | 3.13702 | 1.44E−24 |
| | sialyltranferase 1 | | | |
| RHOJ | ras homolog family member J | 8.78103 | 3.13439 | 3.91E−10 |
| ZBTB8B | zinc finger and BTB domain containing 8B | 8.77744 | 3.1338 | 0.002569 |
| PIK3R3 | phosphoinositide-3-kinase_regulatory | 8.73344 | 3.12655 | 1.04E−59 |
| | subunit 3 (gamma) | | | |
| TRPC5OS | TRPC5 opposite strand | 8.72582 | 3.12529 | 0.003814 |
| HS3ST1 | heparan sulfate (glucosamine) 3-O- | 8.69744 | 3.12059 | 5.29E−07 |
| | sulfotransferase 1 | | | |
| LRMP | lymphoid-restricted membrane protein | 8.64011 | 3.11105 | 9.22E−05 |
| CASC9 | cancer susceptibility candidate 9 | 8.63556 | 3.11029 | 0.006985 |
| | (non-protein coding) | | | |
| EPPK1 | epiplakin 1 | 8.63311 | 3.10988 | 1.71E−19 |
| LGALS9 | lectin_galactoside-binding_soluble_9 | 8.63095 | 3.10952 | 5.69E−10 |
| TNNT1 | troponin T type 1 (skeletal_slow) | 8.59573 | 3.10362 | 2.35E−65 |
| CASKIN1 | CASK interacting protein 1 | 8.58239 | 3.10138 | 1.29E−09 |
| EFNA1 | ephrin-A1 | 8.58233 | 3.10137 | 1.97E−06 |
| EPHA4 | EPH receptor A4 | 8.53032 | 3.0926 | 8.89E−171 |
| FUT1 | fucosyltransferase 1 (galactoside 2-alpha- | 8.52772 | 3.09216 | 2.02E−08 |
| | L-fucosyltransferase_H blood group) | | | |
| CD274 | CD274 molecule | 8.52725 | 3.09208 | 5.00E−85 |
| ADAMTS15 | ADAM metallopeptidase with | 8.52394 | 3.09152 | 2.67E−62 |
| | thrombospondin type 1 motif_15 | | | |
| MYH15 | myosin_heavy chain 15 | 8.51272 | 3.08962 | 2.63E−11 |
| ZBED9 | zinc finger_BED-type containing 9 | 8.47087 | 3.08251 | 2.57E−06 |
| ZFR2 | zinc finger RNA binding protein 2 | 8.46688 | 3.08183 | 0.008776 |
| SLC5A12 | solute carrier family 5 | 8.45837 | 3.08038 | 1.56E−12 |
| | (sodium/monocarboxylate | | | |
| | cotransporter)_member 12 | | | |
| HIST1H4E | histone cluster 1_H4e | 8.45198 | 3.07929 | 3.48E−05 |
| SLC28A3 | solute carrier family 28 (concentrative | 8.43864 | 3.07701 | 5.17E−19 |
| | nucleoside transporter)_member 3 | | | |
| TLR2 | toll-like receptor 2 | 8.42117 | 3.07402 | 1.85E−06 |
| MIR450A2 | microRNA 450a-2 | 8.42064 | 3.07393 | 0.009071 |
| TRHDE-AS1 | TRHDE antisense RNA 1 | 8.39482 | 3.0695 | 5.85E−07 |
| FAM49A | family with sequence similarity | 8.39209 | 3.06903 | 4.98E−09 |
| | 49_member A | | | |
| DTX4 | deltex 4_E3 ubiquitin ligase | 8.35054 | 3.06187 | 7.50E−11 |
| FRZB | frizzled-related protein | 8.34771 | 3.06138 | 2.64E−24 |
| LOC644838 | uncharacterized LOC644838 | 8.33025 | 3.05836 | 0.000163 |
| XKR6 | XK_Kell blood group complex subunit- | 8.3224 | 3.057 | 1.57E−09 |
| | related family_member 6 | | | |
| SERTAD4 | SERTA domain containing 4 | 8.32131 | 3.05681 | 9.53E−05 |
| OR10A3 | olfactory receptor_family 10_subfamily | 8.313 | 3.05537 | 0.000849 |
| | A_member 3 | | | |
| GNGT2 | guanine nucleotide binding protein (G | 8.2804 | 3.0497 | 3.49E−05 |
| | protein)_gamma transducing activity | | | |
| | polypeptide 2 | | | |
| MIR548AO | microRNA 548ao | 8.26864 | 3.04765 | 4.29E−06 |
| SLC29A2 | solute carrier family 29 (equilibrative | 8.25295 | 3.04491 | 7.15E−27 |
| | nucleoside transporter)_member 2 | | | |
| BAI1 | adhesion G protein-coupled receptor B1 | 8.24089 | 3.0428 | 2.23E−05 |
| SAMD12 | sterile alpha motif domain containing 12 | 8.23158 | 3.04117 | 7.76E−74 |
| GUCA1A | guanylate cyclase activator 1A (retina) | 8.22605 | 3.0402 | 4.66E−05 |
| EFR3B | EFR3 homolog B | 8.1885 | 3.0336 | 9.94E−28 |
| LRCH2 | leucine-rich repeats and calponin homology | 8.18147 | 3.03236 | 5.11E−15 |
| | (CH) domain containing 2 | | | |
| ZDHHC11 | zinc finger_DHHC-type containing 11 | 8.15639 | 3.02793 | 9.38E−08 |
| ICAM5 | intercellular adhesion molecule | 8.12 | 3.02148 | 3.39E−16 |
| | 5_telencephalin | | | |
| PYY2 | peptide YY_2 (pseudogene) | 8.11848 | 3.02121 | 2.82E−06 |
| GNG4 | guanine nucleotide binding protein | 8.10724 | 3.01921 | 0.0006 |
| | (G protein)_gamma 4 | | | |
| RASEF | RAS and EF-hand domain containing | 8.09471 | 3.01698 | 1.21E−05 |
| ANKRD1 | ankyrin repeat domain 1 (cardiac muscle) | 8.08955 | 3.01606 | 9.99E−26 |
| SBK1 | SH3 domain binding kinase 1 | 8.07314 | 3.01313 | 3.63E−07 |
| KISS1 | KiSS-1 metastasis-suppressor | 8.05001 | 3.00899 | 8.23E−06 |
| PTPN7 | protein tyrosine phosphatase_non-receptor | 8.04404 | 3.00792 | 3.46E−05 |
| | type 7 | | | |
| KIAA1804 | mixed lineage kinase 4 | 8.03986 | 3.00717 | 0.000206 |
| LCT | lactase | 8.03117 | 3.00561 | 0.002301 |
| IQSEC3 | IQ motif and Sec7 domain 3 | 8.01843 | 3.00332 | 0.000208 |
| CXCL14 | chemokine (C-X-C motif) ligand 14 | 7.97918 | 2.99624 | 4.67E−05 |
| SLC6A16 | solute carrier family 6_member 16 | 7.97901 | 2.99621 | 1.02E−14 |
| PLCXD2 | phosphatidylinositol-specific phospholipase | 7.96315 | 2.99334 | 0.000163 |
| | C_X domain containing 2 | | | |
| THBD | thrombomodulin | 7.94661 | 2.99034 | 8.69E−13 |
| NRGN | neurogranin (protein kinase C | 7.94254 | 2.9896 | 6.09E−13 |
| | substrate_RC3) | | | |
| MAPK15 | mitogen-activated protein kinase 15 | 7.91544 | 2.98467 | 1.72E−10 |
| TSPEAR-AS1 | TSPEAR antisense RNA 1 | 7.9002 | 2.98189 | 6.21E−08 |
| TMEM52 | transmembrane protein 52 | 7.89862 | 2.9816 | 2.09E−10 |
| MIR503 | microRNA 503 | 7.89796 | 2.98148 | 2.97E−09 |
| FBP2 | fructose-1_6-bisphosphatase 2 | 7.86867 | 2.97612 | 8.34E−08 |
| OR5E1P | olfactory receptor_family 5_subfamily | 7.86818 | 2.97603 | 8.61E−06 |
| | E_member 1 pseudogene | | | |
| GS1-24F4.2 | uncharacterized LOC100652791 | 7.85456 | 2.97353 | 7.22E−06 |
| CX3CL1 | chemokine (C-X3-C motif) ligand 1 | 7.85156 | 2.97298 | 0.00914 |
| PLA2G3 | phospholipase A2_group III | 7.82435 | 2.96797 | 0.00959 |
| STK32B | serine/threonine kinase 32B | 7.81351 | 2.96597 | 1.02E−33 |
| NR2F2 | nuclear receptor subfamily 2_group | 7.81139 | 2.96558 | 1.03E−214 |
| | F_member 2 | | | |
| DPF3 | D4_zinc and double PHD fingers_family 3 | 7.8095 | 2.96523 | 5.87E−13 |
| MGARP | mitochondria-localized glutamic acid-rich | 7.79257 | 2.9621 | 1.73E−30 |
| | protein | | | |
| BTBD11 | BTB (POZ) domain containing 11 | 7.74567 | 2.95339 | 2.81E−07 |
| SYNPO2L | synaptopodin 2-like | 7.73698 | 2.95177 | 5.43E−09 |
| SEP3 | septin 3 | 7.66853 | 2.93895 | 3.40E−06 |
| SORL1 | sortilin-related receptor_L(DLR class) A | 7.65048 | 2.93555 | 1.07E−09 |
| | repeats containing | | | |
| MYOZ3 | myozenin 3 | 7.63792 | 2.93318 | 4.21E−27 |
| MIR7851 | microRNA 7851 | 7.62655 | 2.93103 | 0.007612 |
| CNGA1 | cyclic nucleotide gated channel alpha 1 | 7.61086 | 2.92806 | 0.002809 |
| ZCCHC5 | zinc finger_CCHC domain containing 5 | 7.60021 | 2.92604 | 1.23E−07 |
| C14orf105 | chromosome 14 open reading frame 105 | 7.59084 | 2.92426 | 0.009166 |
| ZNF488 | zinc finger protein 488 | 7.5507 | 2.91661 | 1.69E−05 |
| HES7 | hes family bHLH transcription factor 7 | 7.52379 | 2.91146 | 0.000368 |
| CCDC81 | coiled-coil domain containing 81 | 7.51863 | 2.91047 | 3.24E−22 |
| TCEAL7 | transcription elongation factor A (SII)-like 7 | 7.5155 | 2.90987 | 2.66E−13 |
| FRMPD4 | FERM and PDZ domain containing 4 | 7.50957 | 2.90873 | 6.14E−17 |
| CA11 | carbonic anhydrase XI | 7.49039 | 2.90504 | 4.39E−62 |
| GAD1 | glutamate decarboxylase 1 (brain_67 kDa) | 7.48758 | 2.9045 | 1.51E−05 |
| MARCHF3 | membrane-associated ring finger (C3HC4) | 7.47296 | 2.90168 | 3.24E−35 |
| | 3_E3 ubiquitin protein ligase | | | |
| MIR503HG | MIR503 host gene | 7.46654 | 2.90044 | 8.97E−11 |
| NRTN | neurturin | 7.46587 | 2.90031 | 0.005609 |
| PKNOX2 | PBX/knotted 1 homeobox 2 | 7.43731 | 2.89478 | 2.23E−05 |
| TMEM156 | transmembrane protein 156 | 7.42052 | 2.89152 | 0.001671 |
| HHEX | hematopoietically expressed homeobox | 7.41281 | 2.89002 | 1.56E−37 |
| OBSCN | obscurin_cytoskeletal calmodulin and titin- | 7.37136 | 2.88193 | 4.80E−11 |
| | interacting RhoGEF | | | |
| SDPR | serum deprivation response | 7.36482 | 2.88065 | 2.55E−23 |
| PKDCC | protein kinase domain | 7.35375 | 2.87848 | 2.89E−30 |
| | containing_cytoplasmic | | | |
| LOC101926963 | uncharacterized LOC101926963 | 7.30888 | 2.86965 | 1.84E−07 |
| PPP1R9A | protein phosphatase 1_regulatory subunit | 7.30716 | 2.86931 | 0.00519 |
| | 9A | | | |
| CAMK2N1 | calcium/calmodulin-dependent protein | 7.29749 | 2.8674 | 9.32E−95 |
| | kinase II inhibitor 1 | | | |
| MTL5 | metallothionein-like 5_testis-specific | 7.29061 | 2.86604 | 1.26E−23 |
| | (tesmin) | | | |
| COLEC10 | collectin sub-family member 10 (C-type | 7.27986 | 2.86391 | 9.04E−10 |
| | lectin) | | | |
| MAMDC2 | MAM domain containing 2 | 7.27224 | 2.8624 | 4.49E−08 |
| CGN | cingulin | 7.26227 | 2.86042 | 0.002144 |
| KIF25 | kinesin family member 25 | 7.2597 | 2.85991 | 0.005232 |
| GFRA2 | GDNF family receptor alpha 2 | 7.16685 | 2.84134 | 9.87E−06 |
| TSPEAR | thrombospondin-type laminin G domain and | 7.16581 | 2.84113 | 0.008624 |
| | EAR repeats | | | |
| HIST1H2AE | histone cluster 1_H2ae | 7.15846 | 2.83965 | 7.07E−08 |
| MAST1 | microtubule associated serine/threonine | 7.10823 | 2.82949 | 7.37E−06 |
| | kinase 1 | | | |
| PCP2 | Purkinje cell protein 2 | 7.10074 | 2.82797 | 0.000883 |
| RAC3 | ras-related C3 botulinum toxin substrate 3 | 7.09951 | 2.82772 | 9.54E−62 |
| | (rho family_small GTP binding protein | | | |
| | Rac3) | | | |
| JAG2 | jagged 2 | 7.0975 | 2.82731 | 1.53E−11 |
| AFF3 | AF4/FMR2 family_member 3 | 7.08737 | 2.82525 | 2.06E−15 |
| FGFBP3 | fibroblast growth factor binding protein 3 | 7.0854 | 2.82485 | 8.65E−52 |
| NAALAD2 | N-acetylated alpha-linked acidic dipeptidase | 7.07799 | 2.82334 | 5.08E−12 |
| | 2 | | | |
| TMEM184A | transmembrane protein 184A | 7.0749 | 2.82271 | 1.54E−20 |
| PM20D2 | peptidase M20 domain containing 2 | 7.06216 | 2.82011 | 5.26E−17 |
| RAB38 | RAB38_member RAS oncogene family | 7.05771 | 2.8192 | 1.03E−08 |
| RET | ret proto-oncogene | 7.05135 | 2.8179 | 0.000163 |
| HTRA4 | HtrA serine peptidase 4 | 7.04583 | 2.81677 | 7.74E−07 |
| LINC01096 | long intergenic non-protein coding RNA | 7.04232 | 2.81605 | 0.008893 |
| | 1096 | | | |
| SRCRB4D | scavenger receptor cysteine rich | 7.02593 | 2.81269 | 1.07E−17 |
| | family_4 domains | | | |
| SERTAD4-AS1 | SERTAD4 antisense RNA 1 | 7.01377 | 2.81019 | 0.000178 |
| AMN | amnion associated transmembrane protein | 7.0109 | 2.8096 | 1.99E−05 |
| NAP1L2 | nucleosome assembly protein 1-like 2 | 7.00682 | 2.80876 | 6.17E−07 |
| P2RX6P | purinergic receptor P2X_ligand gated ion | 7.0057 | 2.80853 | 8.55E−05 |
| | channel_6 pseudogene | | | |
| PADI2 | peptidyl arginine deiminase_type II | 6.99644 | 2.80662 | 3.36E−09 |
| NEDD4L | neural precursor cell | 6.96899 | 2.80095 | 5.91E−83 |
| | expressed_developmentally down-regulated | | | |
| | 4-like_E3 ubiquitin protein ligase | | | |
| RASGEF1A | RasGEF domain family_member 1A | 6.96098 | 2.79929 | 0.001206 |
| MIR3648 | microRNA 3648-1 | 6.95794 | 2.79866 | 8.24E−06 |
| MIR1204 | microRNA 1204 | 6.93017 | 2.79289 | 1.30E−05 |
| SNORD116-28 | small nucleolar RNA_C/D box 116-28 | 6.92906 | 2.79266 | 1.24E−07 |
| RBP7 | retinol binding protein 7_cellular | 6.87958 | 2.78232 | 3.79E−08 |
| PIK3C2B | phosphatidylinositol-4-phosphate | 6.85849 | 2.77789 | 1.86E−34 |
| | 3-kinase_catalytic subunit type 2 beta | | | |
| SLC4A11 | solute carrier family 4_sodium borate | 6.83969 | 2.77393 | 3.99E−10 |
| | transporter_member 11 | | | |
| ISYNA1 | inositol-3-phosphate synthase 1 | 6.83556 | 2.77306 | 1.30E−19 |
| SALL2 | spalt-like transcription factor 2 | 6.82534 | 2.7709 | 4.21E−10 |
| MIR3687 | microRNA 3687-1 | 6.81026 | 2.76771 | 1.02E−06 |
| SOX5 | SRY (sex determining region Y)-box 5 | 6.76692 | 2.7585 | 0.001343 |
| FOXL1 | forkhead box L1 | 6.74075 | 2.75291 | 4.01E−101 |
| AC093375.1 | NA | 6.73959 | 2.75266 | 0.000186 |
| PLXDC1 | plexin domain containing 1 | 6.7358 | 2.75185 | 3.23E−10 |
| APOE | apolipoprotein E | 6.71948 | 2.74835 | 8.58E−16 |
| HID1 | HID1 domain containing | 6.71478 | 2.74734 | 1.31E−07 |
| SSUH2 | ssu-2 homolog (C. elegans) | 6.71431 | 2.74724 | 0.006498 |
| ABCA12 | ATP-binding cassette_sub-family A | 6.69391 | 2.74285 | 0.000933 |
| | (ABC1)_member 12 | | | |
| OLFM2 | olfactomedin 2 | 6.68636 | 2.74122 | 4.43E−22 |
| GCA | grancalcin_EF-hand calcium binding | 6.68066 | 2.73999 | 2.21E−23 |
| | protein | | | |
| MAGEL2 | melanoma antigen family L2 | 6.67399 | 2.73855 | 6.63E−39 |
| LINC00920 | long intergenic non-protein coding RNA 920 | 6.67311 | 2.73836 | 7.44E−08 |
| SLC40A1 | solute carrier family 40 (iron-regulated | 6.66535 | 2.73668 | 8.07E−16 |
| | transporter)_member 1 | | | |
| MUC19 | mucin 19_oligomeric | 6.65242 | 2.73388 | 0.001879 |
| GRAP2 | GRB2-related adaptor protein 2 | 6.62458 | 2.72783 | 8.63E−06 |
| HOXB6 | homeobox B6 | 6.60748 | 2.7241 | 3.58E−47 |
| ITPRIPL1 | inositol 1_4_5-trisphosphate receptor | 6.59508 | 2.72139 | 4.78E−18 |
| | interacting protein-like 1 | | | |
| LOC100996351 | uncharacterized LOC100996351 | 6.59097 | 2.72049 | 0.00819 |
| F2RL2 | coagulation factor II (thrombin) receptor- | 6.56193 | 2.71412 | 5.61E−09 |
| | like 2 | | | |
| WDR65 | cilia and flagella associated protein 57 | 6.5512 | 2.71176 | 5.50E−05 |
| AP1M2 | adaptor-related protein complex 1_mu 2 | 6.55007 | 2.71151 | 4.62E−09 |
| | subunit | | | |
| PLP1 | proteolipid protein 1 | 6.54975 | 2.71144 | 3.53E−11 |
| SLC6A17 | solute carrier family 6 (neutral amino acid | 6.54662 | 2.71075 | 2.30E−09 |
| | transporter)_member 17 | | | |
| SALL1 | spalt-like transcription factor 1 | 6.52818 | 2.70668 | 0.000106 |
| TRIM17 | tripartite motif containing 17 | 6.51882 | 2.70461 | 1.10E−25 |
| CXorf57 | chromosome X open reading frame 57 | 6.51832 | 2.7045 | 3.14E−11 |
| ELF3 | E74-like factor 3 (ets domain transcription | 6.46482 | 2.69261 | 0.000323 |
| | factor_epithelial-specific ) | | | |
| CNIH2 | cornichon family AMPA receptor auxiliary | 6.44915 | 2.68911 | 1.14E−22 |
| | protein 2 | | | |
| C15orf48 | chromosome 15 open reading frame 48 | 6.44795 | 2.68884 | 6.06E−08 |
| LINGO1 | leucine rich repeat and Ig domain | 6.43861 | 2.68675 | 1.73E−08 |
| | containing 1 | | | |
| CLDN11 | claudin 11 | 6.42987 | 2.68479 | 8.10E−12 |
| PLEKHG3 | pleckstrin homology domain containing_family | 6.42773 | 2.68431 | 4.08E−37 |
| | G (with RhoGef domain) member 3 | | | |
| GPR132 | G protein-coupled receptor 132 | 6.41763 | 2.68204 | 5.59E−06 |
| LINC01239 | long intergenic non-protein coding RNA | 6.4166 | 2.68181 | 1.45E−07 |
| | 1239 | | | |
| SPTB | spectrin_beta_erythrocytic | 6.40669 | 2.67958 | 3.70E−15 |
| LINC00649 | long intergenic non-protein coding RNA 649 | 6.4051 | 2.67922 | 7.95E−05 |
| ST6GALNAC1 | ST6 (alpha-N-acetyl-neuraminyl-2_3-beta- | 6.38785 | 2.67533 | 0.004035 |
| | galactosyl-1_3)-N-acetylgalactosaminide | | | |
| | alpha-2_6-sialyltransferase 1 | | | |
| STOX2 | storkhead box 2 | 6.38108 | 2.6738 | 2.61E−05 |
| HOXB5 | homeobox B5 | 6.37811 | 2.67313 | 7.00E−11 |
| HBQ1 | hemoglobin_theta 1 | 6.37449 | 2.67231 | 0.003817 |
| SORBS1 | sorbin and SH3 domain containing 1 | 6.36597 | 2.67038 | 5.94E−12 |
| DHDH | dihydrodiol dehydrogenase (dimeric) | 6.35821 | 2.66862 | 0.000693 |
| MYOZ2 | myozenin 2 | 6.34034 | 2.66456 | 2.49E−07 |
| MMP23A | matrix metallopeptidase 23A (pseudogene) | 6.31994 | 2.65991 | 0.000145 |
| PDE10A | phosphodiesterase 10A | 6.31801 | 2.65947 | 4.38E−05 |
| HEY1 | hes-related family bHLH transcription factor | 6.30572 | 2.65666 | 1.47E−10 |
| | with YRPW motif 1 | | | |
| CTXN1 | cortexin 1 | 6.30309 | 2.65606 | 2.29E−39 |
| EDN1 | endothelin 1 | 6.30056 | 2.65548 | 2.72E−51 |
| PKD1L1 | polycystic kidney disease 1 like 1 | 6.29078 | 2.65324 | 2.76E−09 |
| LRRC7 | leucine rich repeat containing 7 | 6.28608 | 2.65216 | 0.003815 |
| LIMS3-LOC44089 | LIMS3-LOC440895 readthrough | 6.2829 | 2.65143 | 8.95E−10 |
| PLEKHA6 | pleckstrin homology domain | 6.27972 | 2.6507 | 7.09E−06 |
| | containing_family A member 6 | | | |
| POU3F1 | POU class 3 homeobox 1 | 6.26698 | 2.64777 | 0.002473 |
| AMH | anti-Mullerian hormone | 6.25002 | 2.64386 | 7.65E−10 |
| PCLO | piccolo presynaptic cytomatrix protein | 6.23941 | 2.64141 | 3.62E−08 |
| MYOZ1 | myozenin 1 | 6.21502 | 2.63576 | 3.95E−05 |
| CCDC78 | coiled-coil domain containing 78 | 6.21145 | 2.63493 | 3.59E−10 |
| CCDC85A | coiled-coil domain containing 85A | 6.16961 | 2.62518 | 2.43E−05 |
| PRKX | protein kinase_X-linked | 6.1588 | 2.62265 | 1.00E−48 |
| VEPH1 | ventricular zone expressed PH domain- | 6.15552 | 2.62188 | 7.39E−69 |
| | containing 1 | | | |
| DDX26B | DEAD/H (Asp-Glu-Ala-Asp/His) box | 6.12322 | 2.61429 | 2.74E−09 |
| | polypeptide 26B | | | |
| COCH | cochlin | 6.1044 | 2.60985 | 0.000474 |
| MYH10 | myosin_heavy chain 10_non-muscle | 6.09616 | 2.6079 | 3.63E−52 |
| PDGFD | platelet derived growth factor D | 6.08687 | 2.6057 | 4.95E−06 |
| LINC00704 | long intergenic non-protein coding RNA 704 | 6.08522 | 2.60531 | 5.99E−05 |
| PHACTR1 | phosphatase and actin regulator 1 | 6.07599 | 2.60312 | 0.000104 |
| COL6A4P2 | collagen_type VI_alpha 4 pseudogene 2 | 6.06232 | 2.59987 | 0.005576 |
| TFAP2A | transcription factor AP-2 alpha (activating | 6.06161 | 2.5997 | 9.93E−06 |
| | enhancer binding protein 2 alpha) | | | |
| COL17A1 | collagen_type XVII_alpha 1 | 6.0569 | 2.59858 | 1.18E−08 |
| LRP4 | low density lipoprotein receptor-related | 6.05648 | 2.59848 | 3.47E−20 |
| | protein 4 | | | |
| DUSP4 | dual specificity phosphatase 4 | 6.04855 | 2.59659 | 0.006571 |
| MAP3K15 | mitogen-activated protein kinase kinase | 6.03494 | 2.59334 | 2.05E−05 |
| | kinase 15 | | | |
| RAMP2 | receptor (G protein-coupled) activity | 6.03469 | 2.59328 | 4.50E−09 |
| | modifying protein 2 | | | |
| DOK6 | docking protein 6 | 6.0139 | 2.5883 | 4.14E−06 |
| CELF2 | CUGBP_Elav-like family member 2 | 6.00548 | 2.58628 | 4.06E−05 |
| GRASP | GRP1 (general receptor for | 6.00448 | 2.58604 | 4.24E−16 |
| | phosphoinositides 1)-associated scaffold | | | |
| | protein | | | |
| ERICH5 | glutamate-rich 5 | 6.00407 | 2.58594 | 3.75E−07 |
| MFNG | MFNG O-fucosylpeptide 3-beta-N- | 5.99858 | 2.58462 | 0.001964 |
| | acetylglucosaminyltransferase | | | |
| ETS2 | v-ets avian erythroblastosis virus E26 | 5.99193 | 2.58302 | 1.91E−71 |
| | oncogene homolog 2 | | | |
| C21orf90 | TSPEAR antisense RNA 2 | 5.99164 | 2.58295 | 2.26E−09 |
| GABRA3 | gamma-aminobutyric acid (GABA) A | 5.98931 | 2.58239 | 0.002658 |
| | receptor_alpha 3 | | | |
| FZD9 | frizzled class receptor 9 | 5.9784 | 2.57976 | 2.07E−24 |
| PGM5P2 | phosphoglucomutase 5 pseudogene 2 | 5.96739 | 2.5771 | 1.46E−09 |
| FAM179A | family with sequence similarity | 5.96437 | 2.57637 | 0.000582 |
| | 179_member A | | | |
| GPR183 | G protein-coupled receptor 183 | 5.9604 | 2.57541 | 2.09E−10 |
| WFDC10B | WAP four-disulfide core domain 10B | 5.95318 | 2.57366 | 0.007524 |
| SP6 | Sp6 transcription factor | 5.94353 | 2.57132 | 2.26E−07 |
| AMOT | angiomotin | 5.94114 | 2.57074 | 1.51E−12 |
| MAP2K6 | mitogen-activated protein kinase kinase 6 | 5.93744 | 2.56984 | 2.54E−26 |
| TMEFF1 | transmembrane protein with EGF-like and | 5.92769 | 2.56747 | 0.001063 |
| | two follistatin-like domains 1 | | | |
| TPPP | tubulin polymerization promoting protein | 5.92005 | 2.56561 | 2.81E−08 |
| HIST1H3G | histone cluster 1_H3g | 5.91648 | 2.56474 | 0.0055 |
| RASL10B | RAS-like_family 10_member B | 5.91378 | 2.56408 | 3.58E−48 |
| TNFRSF18 | tumor necrosis factor receptor | 5.91054 | 2.56329 | 6.13E−10 |
| | superfamily_member 18 | | | |
| ADAM19 | ADAM metallopeptidase domain 19 | 5.90493 | 2.56192 | 3.09E−75 |
| LOC400863 | NA | 5.90477 | 2.56188 | 0.00776 |
| MLLT11 | myeloid/lymphoid or mixed-lineage | 5.89863 | 2.56038 | 4.89E−56 |
| | leukemia; translocated to_11 | | | |
| NAV2 | neuron navigator 2 | 5.89552 | 2.55962 | 5.06E−31 |
| UPK1B | uroplakin 1B | 5.88932 | 2.5581 | 0.001333 |
| CORO1A | coronin_actin binding protein_1A | 5.87232 | 2.55393 | 2.46E−16 |
| AQP3 | aquaporin 3 (Gill blood group) | 5.86447 | 2.552 | 1.66E−18 |
| OLFML2A | olfactomedin-like 2A | 5.84118 | 2.54626 | 1.29E−12 |
| CBX2 | chromobox homolog 2 | 5.83693 | 2.54521 | 1.72E−76 |
| KIT | v-kit Hardy-Zuckerman 4 feline sarcoma | 5.83693 | 2.54521 | 4.23E−07 |
| | viral oncogene homolog | | | |
| CSDC2 | cold shock domain containing C2_RNA | 5.83673 | 2.54516 | 5.32E−26 |
| | binding | | | |
| CXorf28 | long intergenic non-protein coding RNA | 5.83592 | 2.54496 | 0.000425 |
| | 1546 | | | |
| TBX5 | T-box 5 | 5.82909 | 2.54327 | 0.002357 |
| CDKL2 | cyclin-dependent kinase-like 2 (CDC2- | 5.82222 | 2.54157 | 3.15E−06 |
| | related kinase) | | | |
| TLE4 | transducin-like enhancer of split 4 | 5.79352 | 2.53444 | 7.60E−234 |
| BRSK2 | BR serine/threonine kinase 2 | 5.79187 | 2.53403 | 2.45E−11 |
| MIR1206 | microRNA 1206 | 5.79059 | 2.53371 | 0.009759 |
| CHRNA5 | cholinergic receptor_nicotinic_alpha 5 | 5.76748 | 2.52794 | 4.07E−05 |
| | (neuronal) | | | |
| DLL3 | delta-like 3 (Drosophila) | 5.75549 | 2.52494 | 9.19E−08 |
| IL1B | interleukin 1_beta | 5.73006 | 2.51855 | 4.35E−05 |
| CDK18 | cyclin-dependent kinase 18 | 5.69833 | 2.51054 | 2.31E−08 |
| PODN | podocan | 5.69782 | 2.51041 | 2.97E−22 |
| MEIS2 | Meis homeobox 2 | 5.69502 | 2.5097 | 9.78E−81 |
| SLC35F2 | solute carrier family 35_member F2 | 5.68338 | 2.50675 | 2.12E−11 |
| MAP3K7CL | MAP3K7 C-terminal like | 5.6811 | 2.50617 | 6.77E−23 |
| LTK | leukocyte receptor tyrosine kinase | 5.67763 | 2.50529 | 0.000247 |
| FILIP1L | filamin A interacting protein 1-like | 5.66777 | 2.50278 | 9.35E−14 |
| CASC8 | cancer susceptibility candidate 8 | 5.664 | 2.50182 | 0.003796 |
| | (non-protein coding) | | | |
| ADM5 | adrenomedullin 5 (putative) | 5.64225 | 2.49627 | 4.68E−07 |
| UNC13A | unc-13 homolog A (C. elegans) | 5.61934 | 2.4904 | 4.82E−06 |
| ZNF702P | zinc finger protein 702_pseudogene | 5.57754 | 2.47963 | 1.14E−08 |
| TFEC | transcription factor EC | 5.56777 | 2.4771 | 0.006309 |
| MAML3 | mastermind-like transcriptional coactivator 3 | 5.55493 | 2.47377 | 2.57E−13 |
| STMN3 | stathmin-like 3 | 5.53717 | 2.46915 | 6.59E−20 |
| GRIP2 | glutamate receptor interacting protein 2 | 5.51064 | 2.46222 | 0.0023 |
| RHOU | ras homolog family member U | 5.50972 | 2.46198 | 2.42E−08 |
| POU2F2 | POU class 2 homeobox 2 | 5.49592 | 2.45836 | 9.51E−29 |
| PMAIP1 | phorbol-12-myristate-13-acetate-induced | 5.49059 | 2.45696 | 3.86E−10 |
| | protein 1 | | | |
| FRMD5 | FERM domain containing 5 | 5.48929 | 2.45662 | 7.37E−40 |
| PTN | pleiotrophin | 5.48074 | 2.45437 | 1.09E−11 |
| LOC101929555 | uncharacterized LOC101929555 | 5.45251 | 2.44692 | 0.004989 |
| ASRGL1 | asparaginase like 1 | 5.44303 | 2.44441 | 7.07E−16 |
| AZU1 | azurocidin 1 | 5.43654 | 2.44269 | 0.000389 |
| LINC00319 | long intergenic non-protein coding RNA 319 | 5.4347 | 2.4422 | 0.002249 |
| ST3GAL5 | ST3 beta-galactoside alpha- | 5.43357 | 2.4419 | 3.42E−46 |
| | 2_3-sialyltransferase 5 | | | |
| GDF6 | growth differentiation factor 6 | 5.4242 | 2.43941 | 1.21E−06 |
| MTRNR2L10 | MT-RNR2-like 10 | 5.42292 | 2.43907 | 0.002039 |
| CSRP2 | cysteine and glycine-rich protein 2 | 5.41166 | 2.43607 | 2.13E−32 |
| PRSS35 | protease_serine_35 | 5.40914 | 2.4354 | 4.82E−10 |
| CDCA7 | cell division cycle associated 7 | 5.39476 | 2.43156 | 1.46E−12 |
| RPS6KA1 | ribosomal protein S6 | 5.38501 | 2.42895 | 1.51E−94 |
| | kinase_90 kDa_polypeptide 1 | | | |
| RUNDC3B | RUN domain containing 3B | 5.34867 | 2.41918 | 2.30E−05 |
| RGS2 | regulator of G-protein signaling 2 | 5.34004 | 2.41685 | 3.99E−54 |
| KRTAP5-1 | keratin associated protein 5-1 | 5.33882 | 2.41652 | 0.006121 |
| LINC01358 | long intergenic non-protein coding RNA | 5.33737 | 2.41613 | 0.000225 |
| | 1358 | | | |
| PLS1 | plastin 1 | 5.33723 | 2.41609 | 8.64E−12 |
| RASGRP2 | RAS guanyl releasing protein 2 (calcium | 5.33552 | 2.41563 | 2.81E−05 |
| | and DAG-regulated) | | | |
| ALOXE3 | arachidonate lipoxygenase 3 | 5.32968 | 2.41405 | 1.76E−06 |
| TNFRSF21 | tumor necrosis factor receptor | 5.3223 | 2.41205 | 5.50E−09 |
| | superfamily_member 21 | | | |
| SYNGR1 | synaptogyrin 1 | 5.29258 | 2.40397 | 9.94E−21 |
| RGS9 | regulator of G-protein signaling 9 | 5.27003 | 2.39781 | 0.007409 |
| ZMYND8 | zinc finger_MYND-type containing 8 | 5.25281 | 2.39309 | 4.51E−28 |
| CASS4 | Cas scaffolding protein family member 4 | 5.25179 | 2.39281 | 0.001735 |
| C20orf166-AS1 | C20orf166 antisense RNA 1 | 5.23613 | 2.3885 | 0.002467 |
| FGFR4 | fibroblast growth factor receptor 4 | 5.22833 | 2.38635 | 7.16E−06 |
| MARCKSL1 | MARCKS-like 1 | 5.22101 | 2.38433 | 4.46E−162 |
| TMEM179 | transmembrane protein 179 | 5.21053 | 2.38143 | 0.006078 |
| NPAS2 | neuronal PAS domain protein 2 | 5.18732 | 2.37499 | 7.68E−06 |
| LPPR4 | lipid phosphate phosphatase-related | 5.15317 | 2.36546 | 3.99E−05 |
| | protein type 4 | | | |
| RGS20 | regulator of G-protein signaling 20 | 5.15188 | 2.3651 | 5.03E−27 |
| RPL13AP20 | ribosomal protein L13a pseudogene 20 | 5.14696 | 2.36372 | 9.02E−08 |
| GPRC5C | G protein-coupled receptor_class C_group | 5.13644 | 2.36077 | 1.12E−15 |
| | 5_member C | | | |
| PARD6G | par-6 family cell polarity regulator gamma | 5.11031 | 2.35341 | 9.22E−67 |
| SLC7A14 | solute carrier family 7_member 14 | 5.09623 | 2.34943 | 4.24E−08 |
| NES | nestin | 5.09319 | 2.34857 | 4.94E−05 |
| CADM4 | cell adhesion molecule 4 | 5.07578 | 2.34363 | 4.33E−30 |
| EBF4 | early B-cell factor 4 | 5.07114 | 2.34231 | 4.29E−07 |
| MEIS1-AS3 | MEIS1 antisense RNA 3 | 5.0691 | 2.34173 | 0.006678 |
| LYPD1 | LY6/PLAUR domain containing 1 | 5.06204 | 2.33972 | 9.67E−12 |
| DMRTA1 | DMRT-like family A1 | 5.04649 | 2.33528 | 0.000332 |
| MKRN7P | makorin ring finger protein 7_pseudogene | 5.02265 | 2.32845 | 0.001418 |
| CHRNB2 | cholinergic receptor_nicotinic_beta 2 | 5.0166 | 2.32671 | 0.002487 |
| | (neuronal) | | | |
| RTN4R | reticulon 4 receptor | 5.01187 | 2.32535 | 1.61E−06 |
| NUTM2G | NUT family member 2G | 5.0032 | 2.32285 | 8.01E−13 |

TABLE 4
Genes more highly expressed in BM MSCs compared with HMCs

| Gene | | Fold | Log Fold | |
|---|---|---|---|---|
| Name | Description | Change | Change | p-Adj |
| MEG3 | maternally expressed 3 (non-protein coding) | −35629.9 | −15.1208 | 7.46E−116 |
| FLG | filaggrin | −6300.72 | −12.6213 | 1.68E−64 |
| DYNLT3 | dynein_light_chain_Tctex-type 3 | −4479.74 | −12.1292 | 2.88E−63 |
| CAT | catalase | −4286.84 | −12.0657 | 2.94E−75 |
| EMX2OS | EMX2 opposite strand/antisense RNA | −2329.98 | −11.1861 | 5.58E−51 |
| EYA2 | EYA transcriptional coactivator and | −2121.1 | −11.0506 | 2.10E−69 |
| | phosphatase 2 | | | |
| CTSF | cathepsin F | −2093.35 | −11.0316 | 1.29E−47 |
| IRX3 | iroquois homeobox 3 | −2000.16 | −10.9659 | 6.67E−128 |
| FNDC1 | fibronectin type III domain containing 1 | −1635.26 | −10.6753 | 1.84E−202 |
| EMX2 | empty spiracles homeobox 2 | −1529.98 | −10.5793 | 4.66E−55 |
| EN1 | engrailed homeobox 1 | −1434.27 | −10.4861 | 2.82E−42 |
| COMP | cartilage oligomeric matrix protein | −1343.15 | −10.3914 | 1.95E−89 |
| S100A6 | S100 calcium binding protein A6 | −1267.09 | −10.3073 | 1.14E−203 |
| TEKT4P2 | tektin 4 pseudogene 2 | −1262.44 | −10.302 | 1.48E−38 |
| HSPB2 | heat shock 27 kDa protein 2 | −1165.07 | −10.1862 | 1.34E−39 |
| GSTT1 | glutathione S-transferase theta 1 | −1164.58 | −10.1856 | 1.18E−39 |
| LYNX1 | Ly6/neurotoxin 1 | −1153.42 | −10.1717 | 4.51E−38 |
| NFASC | neurofascin | −1132.03 | −10.1447 | 3.32E−253 |
| LINC00839 | long intergenic non-protein coding RNA 839 | −1026.77 | −10.0039 | 1.19E−37 |
| ZNF662 | zinc finger protein 662 | −965.023 | −9.91442 | 9.55E−46 |
| BHMT2 | betaine--homocysteine S-methyltransferase 2 | −925.315 | −9.8538 | 5.90E−36 |
| SCUBE1 | signal peptide_CUB domain_EGF-like 1 | −872.185 | −9.76849 | 1.92E−39 |
| FGFR2 | fibroblast growth factor receptor 2 | −810.535 | −9.66273 | 1.40E−137 |
| ANKRD20A5P | ankyrin repeat domain 20 family_member | −768.537 | −9.58597 | 4.90E−33 |
| | A5_pseudogene | | | |
| CES1 | carboxylesterase 1 | −764.679 | −9.57871 | 5.24E−33 |
| CHI3L1 | chitinase 3-like 1 (cartilage glycoprotein-39) | −703.37 | −9.45814 | 8.98E−130 |
| FLG-AS1 | FLG antisense RNA 1 | −667.864 | −9.38341 | 1.10E−29 |
| ISLR | immunoglobulin superfamily containing | −627.765 | −9.29408 | 0.00E+00 |
| | leucine-rich repeat | | | |
| LOC400043 | uncharacterized LOC400043 | −617.438 | −9.27015 | 6.34E−56 |
| LINC01133 | long intergenic non-protein coding RNA 1133 | −608.212 | −9.24843 | 8.34E−91 |
| CYP4F35P | cytochrome P450_family | −601.512 | −9.23245 | 4.19E−29 |
| | 4_subfamily F_polypeptide 35_pseudogene | | | |
| GREM2 | gremlin 2_DAN family BMP antagonist | −598.256 | −9.22462 | 2.08E−126 |
| ANKRD30B | ankyrin repeat domain 30B | −579.225 | −9.17798 | 1.31E−29 |
| PPP1R14C | protein phosphatase 1_regulatory (inhibitor) | −552.557 | −9.10998 | 2.51E−29 |
| | subunit 14C | | | |
| FPR1 | formyl peptide receptor 1 | −489.04 | −8.93381 | 3.56E−27 |
| LINC01268 | long intergenic non-protein coding RNA 1268 | −449.046 | −8.81072 | 1.71E−74 |
| KRT14 | keratin 14_type I | −443.758 | −8.79363 | 7.01E−63 |
| TDRD9 | tudor domain containing 9 | −436.358 | −8.76937 | 6.87E−26 |
| ZNF300P1 | zinc finger protein 300 pseudogene 1 | −420.677 | −8.71657 | 1.01E−30 |
| | (functional) | | | |
| FAM225A | family with sequence similarity 225_member | −400.542 | −8.64581 | 1.47E−25 |
| | A (non-protein coding) | | | |
| FAM180A | family with sequence similarity 180_member | −380.312 | −8.57104 | 7.93E−67 |
| | A | | | |
| CCDC36 | coiled-coil domain containing 36 | −352.867 | −8.46298 | 4.80E−24 |
| CH25H | cholesterol 25-hydroxylase | −352.664 | −8.46215 | 1.70E−23 |
| CCKAR | cholecystokinin A receptor | −324.76 | −8.34323 | 2.32E−22 |
| KRBOX1 | KRAB box domain containing 1 | −322.749 | −8.33427 | 4.54E−23 |
| CCDC144B | coiled-coil domain containing 144B | −315.525 | −8.30161 | 6.20E−23 |
| | (pseudogene) | | | |
| LINC00856 | long intergenic non-protein coding RNA 856 | −313.304 | −8.29142 | 9.69E−23 |
| CSTA | cystatin A (stefin A) | −310.748 | −8.2796 | 1.38E−47 |
| FAM225B | family with sequence similarity 225_member | −301.418 | −8.23562 | 2.28E−22 |
| | B (non-protein coding) | | | |
| LINC00865 | long intergenic non-protein coding RNA 865 | −301.073 | −8.23397 | 2.20E−22 |
| CMKLR1 | chemerin chemokine-like receptor 1 | −281.601 | −8.13751 | 4.31E−19 |
| ENPP2 | ectonucleotide | −271.077 | −8.08256 | 3.42E−71 |
| | pyrophosphatase/phosphodiesterase 2 | | | |
| FMOD | fibromodulin | −269.205 | −8.07256 | 3.90E−23 |
| SDR42E1 | short chain dehydrogenase/reductase family | −252.017 | −7.97738 | 2.21E−20 |
| | 42E_member 1 | | | |
| ITGBL1 | integrin_beta-like 1 (with EGF-like repeat | −244.002 | −7.93075 | 6.52E−295 |
| | domains) | | | |
| IBSP | integrin-binding sialoprotein | −240.491 | −7.90984 | 1.41E−19 |
| FAM20A | family with sequence similarity 20_member A | −235.186 | −7.87766 | 1.62E−85 |
| MKRN3 | makorin ring finger protein 3 | −228.014 | −7.83298 | 1.04E−19 |
| NKAPL | NFKB activating protein-like | −218.076 | −7.76869 | 2.56E−19 |
| C5orf63 | chromosome 5 open reading frame 63 | −214.955 | −7.74789 | 2.78E−24 |
| MYBPH | myosin binding protein H | −214.733 | −7.7464 | 6.31E−26 |
| CPXM2 | carboxypeptidase X (M14 family)_member 2 | −211.34 | −7.72342 | 4.82E−22 |
| CECR7 | cat eye syndrome chromosome | −207.364 | −7.69602 | 2.50E−18 |
| | region_candidate 7 (non-protein coding) | | | |
| PCDHGB3 | protocadherin gamma subfamily B_3 | −206.449 | −7.68964 | 2.56E−18 |
| LINC00968 | long intergenic non-protein coding RNA 968 | −205.155 | −7.68057 | 1.65E−129 |
| FAM66B | family with sequence similarity 66_member B | −202.202 | −7.65965 | 3.81E−18 |
| PENK | proenkephalin | −200.898 | −7.65032 | 3.99E−22 |
| KIAA1644 | KIAA1644 | −194.503 | −7.60365 | 9.45E−107 |
| MEOX2 | mesenchyme homeobox 2 | −193.912 | −7.59926 | 3.51E−16 |
| COX7A1 | cytochrome c oxidase subunit VIIa polypeptide | −191.832 | −7.5837 | 2.42E−46 |
| | 1 (muscle) | | | |
| LOC284757 | NA | −189.246 | −7.56412 | 1.36E−21 |
| SGCD | sarcoglycan_delta (35 kDa dystrophin- | −183.534 | −7.5199 | 1.79E−85 |
| | associated glycoprotein) | | | |
| DDX43 | DEAD (Asp-Glu-Ala-Asp) box polypeptide 43 | −181.828 | −7.50643 | 9.97E−20 |
| LOC101927642 | N | −181.224 | −7.50163 | 3.36E−22 |
| LRRK2 | leucine-rich repeat kinase 2 | −180.898 | −7.49903 | 1.38E−17 |
| NUPR1 | nuclear protein_transcriptional regulator_1 | −178.489 | −7.47969 | 8.60E−126 |
| LOC101929369 | NA | −157.878 | −7.30267 | 7.09E−25 |
| DLX6-AS1 | DLX6 antisense RNA 1 | −154.645 | −7.27282 | 2.04E−21 |
| PCDHGA3 | protocadherin gamma subfamily A_3 | −154.162 | −7.2683 | 5.13E−16 |
| HAS1 | hyaluronan synthase 1 | −153.647 | −7.26348 | 9.36E−40 |
| M1AP | meiosis 1 associated protein | −150.851 | −7.23698 | 9.94E−21 |
| HLA-DPA1 | major histocompatibility complex_class | −147.269 | −7.20231 | 3.20E−14 |
| | II_DP alpha 1 | | | |
| DNAJA4 | DnaJ (Hsp40) homolog_subfamily | −142.774 | −7.15759 | 3.81E−82 |
| | A_member 4 | | | |
| PCDHGA12 | protocadherin gamma subfamily A_12 | −142.64 | −7.15623 | 3.66E−41 |
| MEG8 | maternally expressed 8 (non-protein coding) | −142.207 | −7.15185 | 1.69E−15 |
| KRT16 | keratin 16_type I | −140.972 | −7.13926 | 3.82E−67 |
| NRXN2 | neurexin 2 | −140.865 | −7.13817 | 6.15E−187 |
| PTGES | prostaglandin E synthase | −140.439 | −7.1338 | 0.00E+00 |
| C5AR2 | complement component 5a receptor 2 | −139.462 | −7.12373 | 4.43E−15 |
| ECM2 | extracellular matrix protein 2_female organ | −138.933 | −7.11825 | 6.04E−93 |
| | and adipocyte specific | | | |
| FGF7 | fibroblast growth factor 7 | −138.746 | −7.1163 | 5.19E−71 |
| SLC39A4 | solute carrier family 39 (zinc | −138.362 | −7.1123 | 7.14E−41 |
| | transporter)_member 4 | | | |
| OAS2 | 2′-5′-oligoadenylate synthetase 2_69/71 kDa | −136.733 | −7.09522 | 2.01E−31 |
| HOXC-AS1 | HOXC cluster antisense RNA 1 | −135.946 | −7.08689 | 8.21E−20 |
| LINC00506 | long intergenic non-protein coding RNA 506 | −135.81 | −7.08545 | 3.96E−15 |
| CRYAB | crystallin_alpha B | −133.344 | −7.05901 | 0.00E+00 |
| CKM | creatine kinase_muscle | −131.62 | −7.04023 | 5.91E−15 |
| HYDIN | HYDIN_axonemal central pair apparatus | −130.426 | −7.02709 | 8.10E−26 |
| | protein | | | |
| CYP1B1 | cytochrome P450_family 1_subfamily | −128.476 | −7.00536 | 6.34E−95 |
| | B_polypeptide 1 | | | |
| LINC01018 | long intergenic non-protein coding RNA 1018 | −126.369 | −6.9815 | 7.56E−52 |
| NAALADL1 | N-acetylated alpha-linked acidic dipeptidase- | −126.097 | −6.97839 | 8.44E−96 |
| | like 1 | | | |
| FMO3 | flavin containing monooxygenase 3 | −125.887 | −6.97599 | 2.41E−17 |
| KCNJ15 | potassium channel_inwardly rectifying | −125.648 | −6.97324 | 5.50E−29 |
| | subfamily J_member 15 | | | |
| KRT34 | keratin 34_type I | −123.593 | −6.94945 | 1.45E−238 |
| LSP1 | lymphocyte-specific protein 1 | −123.36 | −6.94673 | 1.62E−77 |
| ADAMTSL3 | ADAMTS-like 3 | −122.924 | −6.94162 | 2.78E−14 |
| LOC101927740 | uncharacterized LOC101927740 | −122.513 | −6.93679 | 4.46E−31 |
| LOC441666 | zinc finger protein 91 pseudogene | −121.086 | −6.91989 | 3.57E−14 |
| LINC01114 | long intergenic non-protein coding RNA 1114 | −120.579 | −6.91384 | 4.38E−14 |
| SPESP1 | sperm equatorial segment protein 1 | −118.239 | −6.88556 | 3.68E−13 |
| LTF | lactotransferrin | −116.299 | −6.8617 | 9.29E−14 |
| ZNF572 | zinc finger protein 572 | −113.357 | −6.82473 | 8.77E−14 |
| ENPP4 | ectonucleotide | −112.876 | −6.81859 | 4.04E−25 |
| | pyrophosphatase/phosphodiesterase 4 | | | |
| | (putative) | | | |
| ANKRD29 | ankyrin repeat domain 29 | −111.733 | −6.80391 | 3.07E−41 |
| ZNF736 | zinc finger protein 736 | −110.633 | −6.78964 | 1.31E−13 |
| COL10A1 | collagen_type_X_alpha 1 | −104.652 | −6.70945 | 4.29E−16 |
| DDO | D-aspartate oxidase | −103.847 | −6.69832 | 4.62E−13 |
| LOC400644 | NA | −103.675 | −6.69592 | 3.54E−13 |
| PID1 | phosphotyrosine interaction domain containing | −103.642 | −6.69546 | 9.95E−50 |
| | 1 | | | |
| LINC00654 | long intergenic non-protein coding RNA 654 | −103.64 | −6.69544 | 6.70E−33 |
| INSRR | insulin receptor-related receptor | −101.301 | −6.6625 | 9.58E−13 |
| FOXQ1 | forkhead box Q1 | −100.715 | −6.65413 | 1.23E−12 |
| LOC150381 | NA | −100.34 | −6.64875 | 1.90E−34 |
| CRLF1 | cytokine receptor-like factor 1 | −98.9591 | −6.62876 | 1.19E−124 |
| ZNF208 | zinc finger protein 208 | −98.7165 | −6.62522 | 1.48E−12 |
| HOXD8 | homeobox D8 | −97.5297 | −6.60777 | 1.81E−139 |
| ZNF454 | zinc finger protein 454 | −97.3285 | −6.60479 | 8.60E−21 |
| GPNMB | glycoprotein (transmembrane) nmb | −97.0778 | −6.60107 | 1.59E−129 |
| NDNF | neuron-derived neurotrophic factor | −95.3473 | −6.57512 | 1.41E−64 |
| KRTAP1-5 | keratin associated protein 1-5 | −94.974 | −6.56946 | 6.22E−138 |
| HTR1F | 5-hydroxytryptamine (serotonin) receptor | −94.3421 | −6.55983 | 1.89E−12 |
| | 1F_G protein-coupled | | | |
| ZFP3 | ZFP3 zinc finger protein | −93.8497 | −6.55228 | 1.48E−85 |
| FGF14 | fibroblast growth factor 14 | −93.5198 | −6.5472 | 3.27E−59 |
| HOXD-AS2 | HOXD cluster antisense RNA 2 | −92.3698 | −6.52935 | 5.39E−47 |
| FAM106A | family with sequence similarity 106_member | −90.6541 | −6.5023 | 3.74E−12 |
| | A | | | |
| SFRP2 | secreted frizzled-related protein 2 | −90.2641 | −6.49608 | 6.67E−12 |
| WISP3 | WNT1 inducible signaling pathway protein 3 | −89.3459 | −6.48133 | 1.32E−29 |
| SORBS2 | sorbin and SH3 domain containing 2 | −85.5325 | −6.4184 | 1.14E−65 |
| HRNR | hornerin | −85.3134 | −6.4147 | 1.35E−11 |
| ANGPT4 | angiopoietin 4 | −85.0978 | −6.41105 | 2.39E−14 |
| PSG5 | pregnancy specific beta-1-glycoprotein 5 | −83.3795 | −6.38162 | 4.75E−178 |
| HOXD3 | homeobox D3 | −82.3393 | −6.36351 | 2.07E−25 |
| PAPPA2 | pappalysin 2 | −81.7037 | −6.35233 | 2.07E−13 |
| LOC728819 | NA | −81.3742 | −6.3465 | 1.77E−11 |
| TGFA | transforming growth factor_alpha | −80.5845 | −6.33243 | 4.10E−11 |
| DEPTOR | DEP domain containing MTOR-interacting | −77.9318 | −6.28414 | 2.95E−62 |
| | protein | | | |
| DMGDH | dimethylglycine dehydrogenase | −77.6697 | −6.27928 | 4.55E−26 |
| PTGDR | prostaglandin D2 receptor (DP) | −77.4445 | −6.27509 | 4.87E−11 |
| LOC102724678 | NA | −77.2241 | −6.27098 | 4.89E−14 |
| C20orf197 | chromosome 20 open reading frame 197 | −75.3602 | −6.23573 | 3.84E−36 |
| RUNX3 | runt-related transcription factor 3 | −75.1822 | −6.23232 | 5.89E−122 |
| IRX5 | iroquois homeobox 5 | −75.1677 | −6.23204 | 1.97E−163 |
| TAS1R1 | taste receptor_type 1_member 1 | −75.1036 | −6.23081 | 5.60E−11 |
| ELANE | elastase_neutrophil expressed | −74.1873 | −6.2131 | 8.13E−11 |
| NINJ2 | ninjurin 2 | −72.5478 | −6.18086 | 1.67E−36 |
| FAM198A | family with sequence similarity 198_member | −72.4965 | −6.17984 | 1.80E−10 |
| | A | | | |
| CXADRP3 | coxsackie virus and adenovirus receptor | −72.3675 | −6.17727 | 1.33E−10 |
| | pseudogene 3 | | | |
| COL14A1 | collagen type XIV_alpha 1 | −72.2227 | −6.17438 | 1.61E−32 |
| CLEC3B | C-type lectin domain family 3_member B | −71.9035 | −6.16799 | 2.18E−42 |
| TMEM178B | transmembrane protein 178B | −71.2387 | −6.15459 | 3.10E−19 |
| ITIH5 | inter-alpha-trypsin inhibitor heavy chain | −71.1864 | −6.15353 | 5.61E−10 |
| | family_member 5 | | | |
| PRPH2 | peripherin 2 (retinal degeneration_slow) | −70.98 | −6.14934 | 4.07E−39 |
| ELN | elastin | −70.9303 | −6.14833 | 1.39E−152 |
| KCTD12 | potassium channel tetramerization domain | −70.8271 | −6.14623 | 1.23E−114 |
| | containing 12 | | | |
| DOK5 | docking protein 5 | −70.5136 | −6.13983 | 1.22E−40 |
| LOC100287846 | patched 1 pseudogene | −70.372 | −6.13693 | 1.78E−10 |
| PTPN20B | protein tyrosine phosphatase_non-receptor | −70.0489 | −6.13029 | 1.79E−10 |
| | type 20 | | | |
| WISP2 | WNT1 inducible signaling pathway protein 2 | −69.2811 | −6.11439 | 4.45E−40 |
| DLX3 | distal-less homeobox 3 | −66.5059 | −6.05541 | 1.41E−18 |
| CCDC89 | coiled-coil domain containing 89 | −66.2524 | −6.0499 | 1.20E−23 |
| FPR2 | formyl peptide receptor 2 | −66.0346 | −6.04515 | 3.24E−10 |
| ITGB2 | integrin_beta 2 (complement component 3 | −65.6849 | −6.03749 | 1.16E−93 |
| | receptor 3 and 4 subunit) | | | |
| PPAPDC3 | phosphatidic acid phosphatase type 2 domain | −65.2393 | −6.02767 | 1.66E−153 |
| | containing 3 | | | |
| ELOVL3 | ELOVL fatty acid elongase 3 | −65.1824 | −6.02641 | 1.24E−28 |
| SERPING1 | serpin peptidase inhibitor_clade G (C1 | −64.6895 | −6.01546 | 7.96E−157 |
| | inhibitor)_member 1 | | | |
| ST8SIA1 | ST8 alpha-N-acetyl-neuraminide alpha-2_8- | −62.1154 | −5.95688 | 1.66E−16 |
| | sialyltransferase 1 | | | |
| PCDHGA4 | protocadherin gamma subfamily A_4 | −61.6851 | −5.94685 | 6.57E−22 |
| TP53TG3D | TP53 target 3D | −61.6052 | −5.94498 | 1.08E−09 |
| PRSS30P | protease_serine_30_pseudogene | −61.4529 | −5.94141 | 8.51E−10 |
| GSTM5 | glutathione S-transferase mu 5 | −61.3317 | −5.93856 | 5.36E−13 |
| P2RY6 | pyrimidinergic receptor P2Y_G-protein | −60.6271 | −5.92189 | 1.09E−69 |
| | coupled_6 | | | |
| EGFLAM | EGF-like_fibronectin type III and laminin G | −60.2517 | −5.91293 | 5.44E−38 |
| | domains | | | |
| TNFRSF11B | tumor necrosis factor receptor | −59.9164 | −5.90488 | 1.45E−102 |
| | superfamily_member 11b | | | |
| ALS2CR11 | amyotrophic lateral sclerosis 2 (juvenile) | −59.6645 | −5.8988 | 8.62E−50 |
| | chromosome region_candidate 11 | | | |
| USP32P2 | ubiquitin specific peptidase 32 pseudogene 2 | −59.5653 | −5.8964 | 1.88E−39 |
| KRT81 | keratin 81_type II | −59.3033 | −5.89004 | 3.27E−15 |
| DCHS2 | dachsous cadherin-related 2 | −59.2162 | −5.88792 | 2.11E−11 |
| XG | Xg blood group | −59.1707 | −5.88681 | 2.16E−69 |
| MAFB | v-maf avian musculoaponeurotic fibrosarcoma | −58.753 | −5.87659 | 9.71E−55 |
| | oncogene homolog B | | | |
| LIPC | lipase_hepatic | −57.1242 | −5.83603 | 1.35E−09 |
| ZNF439 | zinc finger protein 439 | −56.9337 | −5.83121 | 8.44E−49 |
| SLC22A15 | solute carrier family 22_member 15 | −56.5498 | −5.82145 | 6.31E−63 |
| TDRD1 | tudor domain containing 1 | −56.2293 | −5.81325 | 5.08E−09 |
| GRM6 | glutamate receptor_metabotropic 6 | −56.1432 | −5.81104 | 2.31E−11 |
| P2RY2 | purinergic receptor P2Y_G-protein coupled_2 | −55.9967 | −5.80727 | 1.68E−34 |
| ACSM5 | acyl-CoA synthetase medium-chain family | −55.4867 | −5.79407 | 2.02E−09 |
| | member 5 | | | |
| SPAG17 | sperm associated antigen 17 | −55.2572 | −5.78809 | 2.69E−16 |
| LOC101927468 | uncharacterized LOC101927468 | −54.9269 | −5.77944 | 2.23E−09 |
| SYT8 | synaptotagmin VIII | −53.8752 | −5.75155 | 2.03E−16 |
| HOXC4 | homeobox C4 | −53.6672 | −5.74597 | 6.76E−89 |
| HOXC10 | homeobox C10 | −52.9838 | −5.72748 | 1.87E−217 |
| SNORD114-10 | small nucleolar RNA C/D box 114-10 | −52.8042 | −5.72258 | 4.03E−09 |
| BARX1 | BARX homeobox 1 | −52.6707 | −5.71893 | 1.83E−10 |
| LINC00664 | long intergenic non-protein coding RNA 664 | −52.6383 | −5.71804 | 8.69E−09 |
| RGL3 | ral guanine nucleotide dissociation stimulator- | −52.0505 | −5.70184 | 3.35E−52 |
| | like 3 | | | |
| ZNF257 | zinc finger protein 257 | −51.9283 | −5.69845 | 1.02E−08 |
| AKR1C2 | aldo-keto reductase family 1_member C2 | −51.819 | −5.69541 | 9.97E−51 |
| HCAR1 | hydroxycarboxylic acid receptor 1 | −51.5214 | −5.6871 | 1.14E−08 |
| ZDHHC15 | zinc finger_DHHC-type containing 15 | −51.0571 | −5.67404 | 1.28E−08 |
| HSPB7 | heat shock 27 kDa protein family_member 7 | −50.9821 | −5.67192 | 1.96E−97 |
| | (cardiovascular) | | | |
| IFI44L | interferon-induced protein 44-like | −50.8431 | −5.66798 | 3.99E−46 |
| POMC | proopiomelanocortin | −50.2343 | −5.6506 | 4.12E−10 |
| DLX5 | distal-less homeobox 5 | −50.0851 | −5.64631 | 3.03E−53 |
| EPGN | epithelial mitogen | −48.8136 | −5.60921 | 2.21E−36 |
| HAGLR | HOXD antisense growth-associated long non- | −47.4406 | −5.56805 | 2.98E−24 |
| | coding RNA | | | |
| NOTUM | notum pectinacetylesterase homolog | −47.2843 | −5.56329 | 7.00E−23 |
| | (Drosophila) | | | |
| ISM1 | isthmin 1_angiogenesis inhibitor | −46.9645 | −5.5535 | 1.98E−17 |
| SFRP4 | secreted frizzled-related protein 4 | −46.9411 | −5.55278 | 4.74E−13 |
| DLX6 | distal-less homeobox 6 | −46.9268 | −5.55234 | 7.46E−74 |
| CCL28 | chemokine (C-C motif) ligand 28 | −46.8501 | −5.54998 | 7.28E−19 |
| APBB1IP | amyloid beta (A4) precursor | −46.7936 | −5.54824 | 3.06E−66 |
| | protein-binding_family B_member 1 | | | |
| | interacting protein | | | |
| NRN1 | neuritin 1 | −46.7933 | −5.54823 | 2.76E−96 |
| ATP1A2 | ATPase_Na+/K+ transporting_alpha 2 | −45.6518 | −5.5126 | 6.32E−08 |
| | polypeptide | | | |
| SLC2A5 | solute carrier family 2 (facilitated | −45.6069 | −5.51118 | 2.46E−27 |
| | glucose/fructose transporter)_member 5 | | | |
| SAMD9L | sterile alpha motif domain containing 9-like | −45.4488 | −5.50617 | 7.48E−108 |
| EPYC | epiphycan | −45.3506 | −5.50305 | 2.66E−08 |
| REM1 | RAS (RAD and GEM)-like GTP-binding 1 | −45.0583 | −5.49372 | 3.23E−08 |
| CYP19A1 | cytochrome P450_family 19_subfamily | −45.004 | −5.49198 | 2.28E−08 |
| | A_polypeptide 1 | | | |
| SEPSECS-AS1 | SEPSECS antisense RNA 1 (head to head) | −44.8986 | −5.4886 | 2.63E−08 |
| IFI30 | interferon_gamma-inducible protein 30 | −43.4309 | −5.44065 | 2.99E−288 |
| HOXC5 | homeobox C5 | −43.3641 | −5.43843 | 2.23E−39 |
| TMEM233 | transmembrane protein 233 | 41.9538 | −5.39073 | 1.91E−07 |
| METTL7B | methyltransferase like 7B | −41.948 | −5.39053 | 1.51E−23 |
| DOK7 | docking protein 7 | −41.8052 | −5.38561 | 2.21E−15 |
| TNNT3 | troponin T type 3 (skeletal_fast) | −41.6502 | −5.38025 | 4.62E−16 |
| LINC00944 | long intergenic non-protein coding RNA 944 | −41.6467 | −5.38013 | 9.97E−08 |
| HOXC8 | homeobox C8 | −40.9363 | −5.35531 | 2.64E−147 |
| RBP4 | retinol binding protein 4_plasma | −40.7777 | −5.34971 | 4.34E−23 |
| FAM27A | family with sequence similarity 27_member C | −40.5416 | −5.34133 | 7.17E−08 |
| KRT86 | keratin 86_type II | −40.3929 | −5.33603 | 3.14E−18 |
| IFI44 | interferon-induced protein 44 | −40.0664 | −5.32432 | 2.25E−105 |
| LCNL1 | lipocalin-like 1 | −39.8641 | −5.31702 | 5.71E−20 |
| HRCT1 | histidine rich carboxyl terminus 1 | −39.6602 | −5.30962 | 4.53E−64 |
| APOL1 | apolipoprotein L_1 | −39.6399 | −5.30888 | 7.88E−165 |
| ZIC4 | Zic family member 4 | −39.6291 | −5.30849 | 4.67E−17 |
| HCG4 | HLA complex group 4 (non-protein coding) | −39.4647 | −5.30249 | 1.68E−07 |
| MRAP2 | melanocortin 2 receptor accessory protein 2 | −39.3374 | −5.29783 | 1.34E−11 |
| CABP1 | calcium binding protein 1 | −39.2854 | −5.29592 | 3.55E−09 |
| LOC100133445 | NA | −39.1418 | −5.29064 | 1.58E−07 |
| SYN3 | synapsin III | −39.0654 | −5.28782 | 1.56E−07 |
| C11orf70 | chromosome 11 open reading frame 70 | −38.8235 | −5.27886 | 1.39E−124 |
| LINC00482 | long intergenic non-protein coding RNA 482 | −38.7606 | −5.27652 | 1.27E−07 |
| ADAMTS5 | ADAM metallopeptidase with thrombospondin | −37.5963 | −5.23252 | 3.03E−51 |
| | type 1 motif 5 | | | |
| APOC3 | apolipoprotein C-III | −37.5229 | −5.2297 | 2.14E−07 |
| ERG | v-ets avian erythroblastosis virus E26 | −37.4574 | −5.22718 | 3.45E−16 |
| | oncogene homolog | | | |
| PCDHGA6 | protocadherin gamma subfamily A_6 | −37.3744 | −5.22398 | 2.20E−28 |
| CIITA | class II_major histocompatibility | −37.3343 | −5.22243 | 9.56E−09 |
| | complex_transactivator | | | |
| ADIRF | adipogenesis regulatory factor | −37.096 | −5.21319 | 1.00E−21 |
| SP7 | Sp7 transcription factor | −36.5771 | −5.19287 | 5.75E−07 |
| PEG3 | paternally expressed 3 | −36.3802 | −5.18508 | 3.42E−07 |
| BHMT | betaine--homocysteine S-methyltransferase | −36.3023 | −5.18199 | 3.42E−07 |
| RARRES3 | retinoic acid receptor responder (tazarotene | −36.2603 | −5.18032 | 3.07E−34 |
| | induced) 3 | | | |
| ERMN | ermin_ERM-like protein | −36.1008 | −5.17396 | 6.53E−41 |
| KRTAP1-1 | keratin associated protein 1-1 | −35.9286 | −5.16706 | 3.30E−74 |
| ABI3BP | ABI family_member 3 (NESH) binding | −35.9144 | −5.16649 | 7.26E−68 |
| | protein | | | |
| ALX1 | ALX homeobox 1 | −35.5028 | −5.14986 | 7.73E−28 |
| HOMER2 | homer scaffolding protein 2 | −35.447 | −5.14759 | 7.88E−50 |
| HSD17B7P2 | hydroxysteroid (17-beta) dehydrogenase 7 | −35.1909 | −5.13713 | 4.15E−18 |
| | pseudogene 2 | | | |
| IFITM10 | interferon induced transmembrane protein 10 | −35.0208 | −5.13014 | 7.03E−87 |
| PSG1 | pregnancy specific beta-1-glycoprotein 1 | −34.8641 | −5.12367 | 1.04E−06 |
| ASTL | astacin-like metallo-endopeptidase (M12 | −34.4342 | −5.10577 | 1.51E−08 |
| | family) | | | |
| CTLA4 | cytotoxic T-lymphocyte-associated protein 4 | −34.2089 | −5.0963 | 3.13E−10 |
| TNFAIP8L3 | tumor necrosis factor_alpha-induced protein | −34.1767 | −5.09494 | 2.09E−38 |
| | 8-like 3 | | | |
| CSF2RB | colony stimulating factor 2 | −34.0391 | −5.08912 | 1.02E−25 |
| | receptor_beta_low-affinity | | | |
| | (granulocyte-macrophage) | | | |
| SUSD3 | sushi domain containing 3 | −33.8605 | −5.08153 | 4.41E−21 |
| KLF8 | Kruppel-like factor 8 | −33.676 | −5.07365 | 6.76E−09 |
| KLF4 | Kruppel-like factor 4 (gut) | −33.4045 | −5.06197 | 3.52E−163 |
| HAS2 | hyaluronan synthase 2 | −33.3869 | −5.06121 | 1.19E−56 |
| LOC100132891 | NA | −33.1397 | −5.05049 | 2.09E−48 |
| EYA4 | EYA transcriptional coactivator and | −33.1124 | −5.0493 | 5.02E−18 |
| | phosphatase 4 | | | |
| LOC100996609 | NA | −33.0553 | −5.04681 | 3.10E−06 |
| C16orf54 | chromosome 16 open reading frame 54 | −32.8202 | −5.03651 | 8.70E−07 |
| ITGB2-AS1 | ITGB2 antisense RNA 1 | −32.6077 | −5.02714 | 1.02E−25 |
| LINC00884 | long intergenic non-protein coding RNA 884 | −32.4197 | −5.0188 | 7.78E−09 |
| PCDHGA7 | protocadherin gamma subfamily A_7 | −32.4112 | −5.01842 | 3.70E−20 |
| TMEM155 | transmembrane protein 155 | −31.9076 | −4.99583 | 7.65E−43 |
| ITGAL | integrin_alpha L (antigen CD11A | −31.8094 | −4.99138 | 1.12E−06 |
| | (p180)_lymphocyte function-associated antigen 1; | | | |
| | alpha polypeptide) | | | |
| SIX2 | SIX homeobox 2 | −31.7605 | −4.98916 | 1.28E−134 |
| ABCA8 | ATP-binding cassette_sub-family A | −31.5103 | −4.97775 | 1.79E−37 |
| | (ABC1)_member 8 | | | |
| ZNF578 | zinc finger protein 578 | −30.6722 | −4.93886 | 6.76E−29 |
| OOEP | oocyte expressed protein | −30.5166 | −4.93152 | 3.47E−06 |
| DUXAP10 | double homeobox A pseudogene 10 | −30.303 | −4.92139 | 4.16E−09 |
| TEKT4 | tektin 4 | −29.2438 | −4.87006 | 4.86E−06 |
| SYNDIG1 | synapse differentiation inducing 1 | −29.2011 | −4.86795 | 1.73E−31 |
| ZIC1 | Zic family member 1 | −28.9793 | −4.85695 | 8.42E−15 |
| RFX8 | RFX family member 8_lacking RFX DNA | −28.8092 | −4.84846 | 4.93E−29 |
| | binding domain | | | |
| PTGDS | prostaglandin D2 synthase 21 kDa (brain) | −28.8045 | −4.84822 | 2.95E−20 |
| MR1 | major histocompatibility complex_class I- | −28.6716 | −4.84155 | 3.59E−47 |
| | related | | | |
| PCDHGA5 | protocadherin gamma subfamily A_5 | −28.5837 | −4.83712 | 1.02E−25 |
| LTBP2 | latent transforming growth factor beta binding | −28.4538 | −4.83055 | 7.30E−60 |
| | protein 2 | | | |
| LINC00478 | mir-99a-let-7c cluster host gene | −28.3982 | −4.82773 | 5.37E−12 |
| IL6 | interleukin 6 | −28.1909 | −4.81716 | 7.58E−67 |
| LINC00922 | long intergenic non-protein coding RNA 922 | −28.1849 | −4.81685 | 7.58E−06 |
| FBLN7 | fibulin 7 | −28.1669 | −4.81593 | 8.77E−28 |
| PAX8-AS1 | PAX8 antisense RNA 1 | −28.1127 | −4.81315 | 9.03E−07 |
| BRINP1 | bone morphogenetic protein/retinoic acid | −28.0874 | −4.81185 | 9.33E−111 |
| | inducible neural-specific 1 | | | |
| IGJ | joining chain of multimeric IgA and IgM | −28.0393 | −4.80938 | 5.41E−10 |
| PCDHGA11 | protocadherin gamma subfamily A_11 | −28.0007 | −4.80739 | 1.55E−40 |
| KANK4 | KN motif and ankyrin repeat domains 4 | −27.9921 | −4.80695 | 6.94E−06 |
| C15orf54 | chromosome 15 open reading frame 54 | −27.7757 | −4.79575 | 5.79E−13 |
| ZNF492 | zinc finger protein 492 | −27.703 | −4.79197 | 1.66E−07 |
| SNTG2 | syntrophin_gamma 2 | −27.6039 | −4.7868 | 5.38E−22 |
| HOXC9 | homeobox C9 | −27.5876 | −4.78595 | 9.32E−28 |
| CPN2 | carboxypeptidase N_polypeptide 2 | −27.5662 | −4.78483 | 2.28E−08 |
| PP12613 | uncharacterized LOC100192379 | −27.2393 | −4.76762 | 8.38E−08 |
| ANGPTL1 | angiopoietin-like 1 | −27.2239 | −4.7668 | 5.53E−11 |
| PODNL1 | podocan-like 1 | −27.1105 | −4.76078 | 3.88E−88 |
| LOC101926935 | uncharacterized LOC101926935 | −27.0989 | −4.76016 | 4.72E−06 |
| LOC388849 | uncharacterized LOC388849 | −26.899 | −4.74948 | 3.25E−48 |
| CD300C | CD300c molecule | −26.7809 | −4.74313 | 5.77E−06 |
| ASB5 | ankyrin repeat and SOCS box containing 5 | −26.5086 | −4.72839 | 1.84E−16 |
| CCNYL2 | cyclin Y-like 2_pseudogene | −26.4971 | −4.72776 | 6.12E−06 |
| ZFYVE28 | zinc finger_FYVE domain containing 28 | −26.4622 | −4.72586 | 3.11E−64 |
| SERINC2 | serine incorporator 2 | −26.3179 | −4.71797 | 5.05E−126 |
| COL15A1 | collagen_type XV_alpha 1 | −26.0413 | −4.70273 | 9.51E−07 |
| SLC30A3 | solute carrier family 30 (zinc | −25.8968 | −4.6947 | 2.23E−07 |
| | transporter)_member 3 | | | |
| COL5A3 | collagen_type V_alpha 3 | −25.7264 | −4.68518 | 1.17E−31 |
| LOC100505718 | NA | −25.717 | −4.68465 | 7.80E−12 |
| FLG2 | filaggrin family member 2 | −25.5466 | −4.67506 | 1.48E−05 |
| SYBU | syntabulin (syntaxin-interacting) | −25.4087 | −4.66725 | 4.29E−21 |
| LINC00578 | long intergenic non-protein coding RNA 578 | −25.2589 | −4.65872 | 2.57E−07 |
| SLC12A1 | solute carrier family 12 | −25.254 | −4.65844 | 9.59E−06 |
| | (sodium/potassium/chloride | | | |
| | transporter)_member 1 | | | |
| OASL | 2′-5′-oligoadenylate synthetase-like | −25.1755 | −4.65395 | 2.35E−07 |
| OLAH | oleoyl-ACP hydrolase | −25.0589 | −4.64725 | 8.67E−06 |
| KRT9 | keratin 9_type I | −25.0061 | −4.64421 | 6.79E−07 |
| PPAP2B | phosphatidic acid phosphatase type 2B | −24.7585 | −4.62985 | 4.73E−24 |
| TM4SF20 | transmembrane 4 L six family member 20 | −24.584 | −4.61965 | 7.35E−16 |
| PCDHGA2 | protocadherin gamma subfamily A_2 | −24.557 | −4.61806 | 1.82E−17 |
| AMPH | amphiphysin | −24.4871 | −4.61395 | 3.66E−81 |
| KCNK15 | potassium channel_two pore domain | −24.4564 | −4.61214 | 2.64E−14 |
| | subfamily K_member 15 | | | |
| HOXA10-AS | HOXA10 antisense RNA | −24.4528 | −4.61193 | 7.23E−30 |
| INSC | inscuteable homolog (Drosophila) | −24.452 | −4.61188 | 1.62E−05 |
| MIR4257 | microRNA 4257 | −24.4166 | −4.60979 | 1.11E−05 |
| HOXC6 | homeobox C6 | −24.4007 | −4.60885 | 1.41E−36 |
| RTP4 | receptor (chemosensory) transporter protein 4 | −24.3581 | −4.60633 | 1.95E−05 |
| GAS1 | growth arrest-specific 1 | −24.0511 | −4.58803 | 9.44E−50 |
| EBF1 | early B-cell factor 1 | −23.9491 | −4.5819 | 3.55E−143 |
| SNTB1 | syntrophin_beta 1 (dystrophin-associated | −23.9123 | −4.57968 | 1.73E−74 |
| | protein A1_59 kDa_basic component 1) | | | |
| ANPEP | alanyl (membrane) aminopeptidase | −23.8821 | −4.57786 | 0.00E+00 |
| C10orf105 | chromosome 10 open reading frame 105 | −23.8719 | −4.57724 | 4.12E−07 |
| PCDHGB1 | protocadherin gamma subfamily B_1 | −23.7715 | −4.57116 | 4.31E−13 |
| COMT | catechol-O-methyltransferase | −23.7198 | −4.56802 | 8.63E−144 |
| CYP7B1 | cytochrome P450_family 7_subfamily | −23.7073 | −4.56726 | 5.30E−07 |
| | B_polypeptide 1 | | | |
| KLHL33 | kelch-like family member 33 | −23.6812 | −4.56567 | 1.63E−05 |
| KLHL13 | kelch-like family member 13 | −23.596 | −4.56047 | 1.50E−44 |
| RAET1E | retinoic acid early transcript 1E | −23.5653 | −4.55859 | 1.34E−06 |
| ABCC3 | ATP-binding cassette_sub-family C | −23.5388 | −4.55697 | 1.55E−32 |
| | (CFTR/MRP)_member 3 | | | |
| PRR34 | proline rich 34 | −23.4808 | −4.55341 | 5.23E−12 |
| LOC100130992 | uncharacterized LOC100130992 | −23.2829 | −4.5412 | 2.47E−26 |
| ISLR2 | immunoglobulin superfamily containing | −23.2065 | −4.53646 | 4.26E−05 |
| | leucine-rich repeat 2 | | | |
| PLAC9 | placenta-specific 9 | −23.1863 | −4.5352 | 7.53E−79 |
| ATE1-AS1 | ATE1 antisense RNA 1 | −22.9836 | −4.52253 | 9.59E−06 |
| ZMYND15 | zinc finger_MYND-type containing 15 | −22.9796 | −4.52228 | 3.63E−15 |
| PRL | prolactin | −22.9438 | −4.52003 | 1.60E−05 |
| GPAT2 | glycerol-3-phosphate acyltransferase | −22.8257 | −4.51259 | 2.15E−15 |
| | 2_mitochondrial | | | |
| SYT11 | synaptotagmin XI | −22.6805 | −4.50338 | 6.30E−20 |
| RTN4RL1 | reticulon 4 receptor-like 1 | −22.6662 | −4.50247 | 8.59E−07 |
| PDK4 | pyruvate dehydrogenase kinase_isozyme 4 | −22.5842 | −4.49724 | 6.18E−13 |
| IGF1 | insulin-like growth factor 1 (somatomedin C) | −22.4869 | −4.49101 | 4.74E−21 |
| COL8A2 | collagen_type VIII_alpha 2 | −22.4439 | −4.48825 | 3.73E−22 |
| C12orf56 | chromosome 12 open reading frame 56 | −22.4084 | −4.48597 | 9.34E−08 |
| CHRDL2 | chordin-like 2 | −22.3783 | −4.48403 | 1.55E−06 |
| MIR10B | microRNA 10b | −22.2523 | −4.47588 | 2.37E−05 |
| IL18R1 | interleukin 18 receptor 1 | −22.2043 | −4.47277 | 1.27E−08 |
| OMD | osteomodulin | −22.1734 | −4.47076 | 2.63E−05 |
| C9orf170 | chromosome 9 open reading frame 170 | −22.1436 | −4.46882 | 9.24E−07 |
| HOXD4 | homeobox D4 | −22.1291 | −4.46787 | 3.19E−29 |
| LINC01060 | long intergenic non-protein coding RNA 1060 | −22.1154 | −4.46698 | 4.42E−05 |
| LOC100130539 | NA | −22.0684 | −4.46391 | 4.11E−14 |
| ASPG | asparaginase | −22.0317 | −4.46151 | 3.52E−05 |
| LOC729296 | uncharacterized LOC729296 | −21.9806 | −4.45816 | 2.94E−05 |
| SPATA41 | spermatogenesis associated 41 (non-protein | −21.9421 | −4.45563 | 1.98E−06 |
| | coding) | | | |
| LRRN4CL | LRRN4 C-terminal like | −21.936 | −4.45523 | 4.64E−41 |
| MYOC | myocilin_trabecular meshwork inducible | −21.9193 | −4.45413 | 5.99E−05 |
| | glucocorticoid response | | | |
| POSTN | periostin_osteoblast specific factor | −21.9072 | −4.45333 | 2.31E−13 |
| FOXF2 | forkhead box F2 | −21.8768 | −4.45133 | 2.78E−103 |
| LYPD5 | LY6/PLAUR domain containing 5 | −21.8023 | −4.44641 | 3.63E−05 |
| ALX4 | ALX homeobox 4 | −21.7607 | −4.44365 | 8.25E−14 |
| HTR7 | 5-hydroxytryptamine (serotonin) receptor | −21.6032 | −4.43317 | 4.24E−12 |
| | 7_adenylate cyclase-coupled | | | |
| MCOLN3 | mucolipin 3 | −21.5099 | −4.42693 | 1.84E−11 |
| NXF3 | nuclear RNA export factor 3 | −21.506 | −4.42667 | 9.32E−10 |
| MFAP5 | microfibrillar associated protein 5 | −21.467 | −4.42405 | 6.48E−64 |
| MALRD1 | MAM and LDL receptor class A domain | −21.4603 | −4.4236 | 3.91E−05 |
| | containing 1 | | | |
| ADAMTS4 | ADAM metallopeptidase with thrombospondin | −21.4536 | −4.42315 | 1.39E−43 |
| | type 1 motif_4 | | | |
| ZNF528 | zinc finger protein 528 | −21.4192 | −4.42083 | 8.50E−35 |
| SLC8A3 | solute carrier family 8 (sodium/calcium | −21.3959 | −4.41926 | 7.10E−05 |
| | exchanger)_member 3 | | | |
| NDUFA4L2 | NADH dehydrogenase (ubiquinone) 1 alpha | −21.3677 | −4.41736 | 2.98E−19 |
| | subcomplex_4-like 2 | | | |
| TRABD2B | TraB domain containing 2B | −21.2105 | −4.40671 | 1.19E−09 |
| SIM1 | single-minded family bHLH transcription | −21.2004 | −4.40602 | 9.35E−06 |
| | factor 1 | | | |
| FAM19A5 | family with sequence similarity 19 (chemokine | −21.1652 | −4.40362 | 3.42E−44 |
| | (C-C motif)-like)_member A5 | | | |
| FAM50B | family with sequence similarity 50_member B | −21.0535 | −4.39599 | 1.01E−50 |
| KCNN4 | potassium channel_calcium activated | −20.9584 | −4.38946 | 2.97E−46 |
| | intermediate/small conductance subfamily N | | | |
| | alpha_member 4 | | | |
| HTR2A | 5-hydroxytryptamine (serotonin) receptor | −20.9571 | −4.38937 | 0.00011 |
| | 2A_G protein-coupled | | | |
| PM20D1 | peptidase M20 domain containing 1 | −20.5974 | −4.36439 | 8.81E−05 |
| LOC100506834 | uncharacterized LOC100506834 | −20.5877 | −4.36371 | 6.39E−17 |
| PLD5 | phospholipase D family_member 5 | −20.5811 | −4.36325 | 0.000159 |
| NR4A2 | nuclear receptor subfamily 4_group | −20.3715 | −4.34848 | 1.39E−29 |
| | A_member 2 | | | |
| BACH2 | BTB and CNC homology 1_basic leucine | −20.2688 | −4.34119 | 2.14E−28 |
| | zipper transcription factor 2 | | | |
| CRIP1 | cysteine-rich protein 1 (intestinal) | −20.2183 | −4.33759 | 1.77E−45 |
| ANGPTL5 | angiopoietin-like 5 | −20.2061 | −4.33672 | 7.79E−05 |
| USP32P1 | ubiquitin specific peptidase 32 pseudogene 1 | −20.1748 | −4.33448 | 6.91E−06 |
| PLSCR4 | phospholipid scramblase 4 | −20.0422 | −4.32497 | 1.83E−45 |
| BACE2 | beta-site APP-cleaving enzyme 2 | −20.0121 | −4.3228 | 5.29E−71 |
| CYP1B1-AS1 | CYP1B1 antisense RNA 1 | −19.9626 | −4.31923 | 2.92E−13 |
| SLC14A2 | solute carrier family 14 (urea | −19.7333 | −4.30256 | 7.17E−05 |
| | transporter)_member 2 | | | |
| POU5F1 | POU class 5 homeobox 1 | −19.6359 | −4.29542 | 1.68E−08 |
| KCND3 | potassium channel_voltage gated Shal related | −19.5189 | −4.2868 | 1.65E−06 |
| | subfamily D_member 3 | | | |
| RHBDL2 | rhomboid veinlet-like 2 (Drosophila) | −19.5079 | −4.28599 | 2.69E−35 |
| CCDC67 | coiled-coil domain containing 67 | −19.222 | −4.26469 | 8.79E−05 |
| ADAMTS2 | ADAM metallopeptidase with thrombospondin | −19.1384 | −4.2584 | 4.81E−197 |
| | type 1 motif_2 | | | |
| ENTPD1-AS1 | ENTPD1 antisense RNA 1 | −19.1196 | −4.25698 | 6.87E−05 |
| MLKL | mixed lineage kinase domain-like | −19.1013 | −4.2556 | 1.91E−88 |
| BMPR1B | bone morphogenetic protein receptor_type IB | −18.9871 | −4.24695 | 1.35E−21 |
| LINC00028 | long intergenic non-protein coding RNA 28 | −18.9553 | −4.24453 | 7.98E−05 |
| MYOT | myotilin | −18.7318 | −4.22742 | 4.74E−10 |
| ADRA2C | adrenoceptor alpha 2C | −18.6355 | −4.21998 | 3.71E−27 |
| HOXC-AS2 | HOXC cluster antisense RNA 2 | −18.5603 | −4.21415 | 1.91E−13 |
| TIMP3 | TIMP metallopeptidase inhibitor 3 | −18.3901 | −4.20086 | 7.89E−33 |
| C21orf119 | URB1 antisense RNA 1 (head to head) | −18.3392 | −4.19686 | 6.85E−48 |
| ANKRD7 | ankyrin repeat domain 7 | −18.2307 | −4.1883 | 0.000179 |
| ANKRD20A9P | ankyrin repeat domain 20 family_member | −18.175 | −4.18388 | 0.000271 |
| | A9_pseudogene | | | |
| NFE2 | nuclear factor_erythroid 2 | −18.1645 | −4.18305 | 1.53E−41 |
| ASS1 | argininosuccinate synthase 1 | −18.1342 | −4.18064 | 1.34E−73 |
| BTLA | B and T lymphocyte associated | −18.1274 | −4.1801 | 1.28E−05 |
| SLC14A1 | solute carrier family 14 (urea | −18.1032 | −4.17817 | 4.65E−33 |
| | transporter)_member 1 (Kidd blood group) | | | |
| ANKRD6 | ankyrin repeat domain 6 | −18.0164 | −4.17124 | 1.65E−85 |
| DMBT1 | deleted in malignant brain tumors 1 | −17.9852 | −4.16874 | 0.000133 |
| LINC00271 | long intergenic non-protein coding RNA 271 | −17.9802 | −4.16834 | 1.18E−05 |
| OR2S2 | olfactory receptor_family 2_subfamily | −17.9513 | −4.16602 | 0.000215 |
| | S_member 2 (gene/pseudogene) | | | |
| SNED 1 | sushi_nidogen and EGF-like domains 1 | −17.8528 | −4.15808 | 8.57E−101 |
| LOC392232 | transient receptor potential cation | −17.7659 | −4.15104 | 0.000183 |
| | channel_subfamily A_member 1 pseudogene | | | |
| KCNT2 | potassium channel sodium activated | −17.5312 | −4.13185 | 3.15E−18 |
| | subfamily T_member 2 | | | |
| RORA | RAR-related orphan receptor A | −17.4834 | −4.12791 | 1.46E−114 |
| TNFSF9 | tumor necrosis factor (ligand) | −17.4353 | −4.12394 | 2.78E−21 |
| | superfamily_member 9 | | | |
| ADH1C | alcohol dehydrogenase 1C (class I)_gamma | −17.4236 | −4.12297 | 0.000163 |
| | polypeptide | | | |
| FBXO39 | F-box protein 39 | −17.3262 | −4.11488 | 0.000153 |
| ZNF595 | zinc finger protein 595 | 17.2323 | −4.10704 | 1.79E−30 |
| LMO7DN | LM07 downstream neighbor | −17.2099 | −4.10517 | 2.90E−17 |
| PI16 | peptidase inhibitor 16 | −17.1836 | −4.10296 | 2.92E−11 |
| EPDR1 | ependymin related 1 | −17.0851 | −4.09467 | 8.65E−36 |
| HLA-DRA | major histocompatibility complex_class | −17.078 | −4.09407 | 0.000417 |
| | II_DR alpha | | | |
| C10orf54 | chromosome 10 open reading frame 54 | −17.0211 | −4.08925 | 2.39E−93 |
| ZNF311 | zinc finger protein 311 | −17.0103 | −4.08834 | 2.37E−10 |
| LINC01119 | long intergenic non-protein coding RNA 1119 | −16.956 | −4.08372 | 7.58E−67 |
| RASSF9 | Ras association (RalGDS/AF-6) domain | −16.9475 | −4.083 | 4.43E−41 |
| | family (N-terminal) member 9 | | | |
| HLA-DRB1 | major histocompatibility complex_class | −16.872 | −4.07656 | 2.00E−08 |
| | II_DR beta 1 | | | |
| HMOX1 | heme oxygenase 1 | −16.8711 | −4.07648 | 2.77E−128 |
| MIRLET7BHG | MIRLET7B host gene | −16.8548 | −4.07509 | 2.54E−22 |
| TRPM3 | transient receptor potential cation | −16.8294 | −4.07291 | 2.12E−13 |
| | channel_subfamily M_member 3 | | | |
| CCDC64B | coiled-coil domain containing 64B | −16.8047 | −4.07079 | 10.000407 |
| HOXA9 | homeobox A9 | −16.7153 | −4.0631 | 4.84E−53 |
| BATF | basic leucine zipper transcription factor_ATF- | −16.7105 | −4.06268 | 0.000383 |
| | like | | | |
| IGFBPL1 | insulin-like growth factor binding protein-like | −16.6703 | −4.05921 | 1.29E−35 |
| | 1 | | | |
| KCNH1 | potassium channel_voltage gated eag related | −16.6561 | −4.05798 | 2.27E−18 |
| | subfamily H_member 1 | | | |
| LPO | lactoperoxidase | −16.5886 | −4.05212 | 1.79E−05 |
| ADCY4 | adenylate cyclase 4 | −16.5671 | −4.05025 | 4.41E−19 |
| ANKRD65 | ankyrin repeat domain 65 | −16.3912 | −4.03485 | 8.58E−49 |
| OLFML1 | olfactomedin-like 1 | −16.3551 | −4.03167 | 2.87E−21 |
| C11orf96 | chromosome 11 open reading frame 96 | −16.2548 | −4.02279 | 3.34E−22 |
| TLE2 | transducin-like enhancer of split 2 | −16.2491 | −4.02229 | 3.65E−36 |
| LOC653602 | uncharacterized LOC653602 | −16.2049 | −4.01836 | 2.06E−10 |
| EVA1C | eva-1 homolog C (C. elegans) | −16.2023 | −4.01813 | 2.64E−72 |
| SATB2-AS1 | SATB2 antisense RNA 1 | −16.0753 | −4.00677 | 0.000254 |
| GBP5 | guanylate binding protein 5 | −16.0594 | −4.00535 | 6.57E−06 |
| IL1R1 | interleukin 1 receptor_type I | −16.0375 | −4.00338 | 5.75E−80 |
| MIR656 | microRNA 656 | −15.983 | −3.99847 | 0.000289 |
| KCNK2 | potassium channel_two pore domain | −15.9332 | −3.99396 | 2.10E−31 |
| | subfamily K_member 2 | | | |
| TNFRSF14 | tumor necrosis factor receptor | −15.913 | −3.99213 | 5.69E−44 |
| | superfamily_member 14 | | | |
| PCDHGA1 | protocadherin gamma subfamily A_1 | −15.866 | −3.98787 | 5.58E−09 |
| CCL20 | chemokine (C-C motif) ligand 20 | −15.8653 | −3.9878 | 6.53E−09 |
| LOC284412 | uncharacterized LOC284412 | −15.8392 | −3.98543 | 5.86E−07 |
| TNFAIP6 | tumor necrosis factor_alpha-induced protein 6 | −15.7201 | −3.97454 | 7.00E−12 |
| ACAN | aggrecan | −15.6611 | −3.96911 | 3.97E−83 |
| VTRNA1-2 | vault RNA 1-2 | −15.6195 | −3.96528 | 3.85E−10 |
| RGN | regucalcin | −15.609 | −3.96431 | 3.76E−23 |
| NR4A1 | nuclear receptor subfamily 4_group | −15.5795 | −3.96158 | 3.99E−95 |
| | A_member 1 | | | |
| TNS4 | tensin 4 | −15.5521 | −3.95904 | 9.17E−22 |
| CFB | complement factor B | −15.4426 | −3.94884 | 5.90E−31 |
| TMEM119 | transmembrane protein 119 | −15.4262 | −3.94731 | 2.09E−59 |
| MIR4271 | microRNA 4271 | −15.3448 | −3.93968 | 0.000385 |
| ABCC9 | ATP-binding cassette_sub-family C | −15.294 | −3.93489 | 6.82E−35 |
| | (CFTR/MRP)_member 9 | | | |
| AGMO | alkylglycerol monooxygenase | −15.2848 | −3.93403 | 0.000102 |
| RIPK3 | receptor-interacting serine-threonine kinase 3 | −15.1926 | −3.9253 | 1.71E−31 |
| SLPI | secretory leukocyte peptidase inhibitor | −15.1322 | −3.91955 | 0.000432 |
| MIR23A | microRNA 23a | −15.1079 | −3.91723 | 0.000424 |
| EBF3 | early B-cell factor 3 | −15.0595 | −3.9126 | 2.02E−102 |
| RGS22 | regulator of G-protein signaling 22 | −15.059 | −3.91255 | 7.45E−05 |
| PRUNE2 | prune homolog 2 (Drosophila) | −15.0164 | −3.90847 | 3.38E−83 |
| A2M | alpha-2-macroglobulin | −15.0025 | −3.90713 | 4.18E−14 |
| LRRC15 | leucine rich repeat containing 15 | −14.8862 | −3.8959 | 1.94E−16 |
| LOC101927650 | uncharacterized LOC101927650 | −14.8779 | −3.8951 | 4.42E−05 |
| LINC00870 | long intergenic non-protein coding RNA 870 | −14.8421 | −3.89162 | 0.000319 |
| LANCL3 | LanC lantibiotic synthetase component C-like | −14.8372 | −3.89115 | 1.68E−08 |
| | 3 (bacterial) | | | |
| SLC6A1 | solute carrier family 6 (neurotransmitter | −14.7885 | −3.8864 | 0.000151 |
| | transporter) member 1 | | | |
| SNORD113-4 | small nucleolar RNA_C/D box 113-4 | −14.7598 | −3.8836 | 0.000496 |
| APOL6 | apolipoprotein L_6 | −14.7294 | −3.88063 | 2.08E−62 |
| CRIP3 | cysteine-rich protein 3 | −14.6825 | −3.87603 | 5.54E−07 |
| ADPRH | ADP-ribosylarginine hydrolase | −14.6571 | −3.87353 | 6.54E−66 |
| PLA2G5 | phospholipase A2_group V | −14.6359 | −3.87144 | 0.001334 |
| LINC00877 | long intergenic non-protein coding RNA 877 | −14.4527 | −3.85327 | 0.000505 |
| FIBIN | fin bud initiation factor homolog (zebrafish) | −14.4064 | −3.84864 | 2.26E−27 |
| LIPI | lipase_member I | −14.3906 | −3.84705 | 0.000612 |
| LINC01121 | long intergenic non-protein coding RNA 1121 | −14.3857 | −3.84656 | 0.000775 |
| ABCA6 | ATP-binding cassette_sub-family A | −14.3797 | −3.84596 | 2.42E−16 |
| | (ABC1)_member 6 | | | |
| LINC00961 | long intergenic non-protein coding RNA 961 | 14.3653 | −3.84452 | 4.16E−29 |
| MLIP | muscular LMNA-interacting protein | −14.299 | −3.83784 | 6.43E−05 |
| TP63 | tumor protein p63 | −14.2856 | −3.83649 | 4.77E−08 |
| MEDAG | mesenteric estrogen-dependent adipogenesis | −14.2853 | −3.83646 | 3.66E−98 |
| FOSB | FBJ murine osteosarcoma viral oncogene | −14.261 | −3.834 | 3.25E−18 |
| | homolog B | | | |
| CCDC144A | coiled-coil domain containing 144A | −14.2522 | −3.83311 | 3.88E−05 |
| ZNF704 | zinc finger protein 704 | −14.252 | −3.83309 | 3.01E−11 |
| FZD1 | frizzled class receptor 1 | −14.1041 | −3.81804 | 2.20E−37 |
| NPR3 | natriuretic peptide receptor 3 | −14.0506 | −3.81256 | 2.04E−32 |
| LRRC6 | leucine rich repeat containing 6 | −13.9759 | −3.80487 | 1.02E−33 |
| LAMA4 | laminin_alpha 4 | −13.9682 | −3.80407 | 3.57E−32 |
| FLJ22447 | uncharacterized LOC400221 | −13.9582 | −3.80304 | 6.71E−23 |
| ANKFN1 | ankyrin-repeat and fibronectin type III domain | −13.9499 | −3.80218 | 9.52E−11 |
| | containing 1 | | | |
| LOC101927524 | NA | −13.947 | −3.80188 | 0.000913 |
| C3 | complement component 3 | −13.9125 | −3.79831 | 7.27E−21 |
| TCHH | trichohyalin | −13.8398 | −3.79075 | 0.000751 |
| TMSB4Y | thymosin beta 4_Y-linked | −13.839 | −3.79067 | 0.001604 |
| PON3 | paraoxonase 3 | −13.7218 | −3.7784 | 0.00108 |
| KRT83 | keratin 83_type II | −13.7094 | −3.77709 | 0.00026 |
| AGT | angiotensinogen (serpin peptidase | −13.702 | −3.77631 | 2.67E−25 |
| | inhibitor_clade A_member 8) | | | |
| CEMIP | cell migration inducing protein_hyaluronan | −13.667 | −3.77262 | 4.15E−30 |
| | binding | | | |
| MIR4297 | microRNA 4297 | −13.6626 | −3.77216 | 0.001069 |
| PSORS1C3 | psoriasis susceptibility 1 candidate 3 (non- | −13.5703 | −3.76238 | 0.001055 |
| | protein coding) | | | |
| ITGA8 | integrin_alpha 8 | −13.5429 | −3.75946 | 2.41E−67 |
| LOC102546299 | uncharacterized LOC102546299 | −13.5364 | −3.75877 | 1.02E−06 |
| GSTM1 | glutathione S-transferase mu 1 | −13.5248 | −3.75754 | 0.001586 |
| MIR6730 | microRNA 6730 | −13.49 | −3.75382 | 9.36E−05 |
| DHX58 | DEXH (Asp-Glu-X-His) box polypeptide 58 | −13.4889 | −3.7537 | 5.11E−29 |
| CXCL16 | chemokine (C-X-C motif) ligand 16 | −13.4676 | −3.75142 | 1.20E−38 |
| GJB5 | gap junction protein_beta 5_31.1 kDa | −13.4535 | −3.74991 | 0.000119 |
| SCIN | scinderin | −13.4499 | −3.74952 | 3.34E−08 |
| CSGALNACT1 | chondroitin sulfate N- | −13.4328 | −3.74769 | 1.50E−70 |
| | acetylgalactosaminyltransferase 1 | | | |
| LOC101928882 | uncharacterized LOC101928882 | −13.4233 | −3.74667 | 0.001191 |
| MSC | musculin | −13.4205 | −3.74637 | 2.37E−56 |
| WEE2 | WEE1 homolog 2 (S. pombe) | −13.4086 | −3.74509 | 0.001064 |
| NR1I2 | nuclear receptor subfamily 1_group | −13.3169 | −3.73519 | 0.000887 |
| | I_member 2 | | | |
| OAS1 | 2′-5′-oligoadenylate synthetase 1_40/46 kDa | −13.282 | −3.7314 | 3.02E−07 |
| LINC01116 | long intergenic non-protein coding RNA 1116 | −13.2627 | −3.7293 | 2.01E−69 |
| VMO1 | vitelline membrane outer layer 1 homolog | −13.2306 | −3.72581 | 7.39E−16 |
| | (chicken) | | | |
| CD4 | CD4 molecule | −13.2111 | −3.72368 | 9.20E−251 |
| SLAMF9 | SLAM family member 9 | −13.208 | −3.72334 | 5.08E−17 |
| COL12A1 | collagen_type XII_alpha 1 | −13.1992 | −3.72238 | 1.82E−30 |
| TBX15 | T-box 15 | −13.1967 | −3.7221 | 2.80E−169 |
| LOC102724224 | NA | −13.1605 | −3.71814 | 3.31E−26 |
| EYA1 | EYA transcriptional coactivator and | −13.1442 | −3.71635 | 9.67E−11 |
| | phosphatase 1 | | | |
| HOXA1 | homeobox A1 | 13.0858 | −3.70993 | 3.29E−34 |
| IL21R | interleukin 21 receptor | −13.0523 | −3.70623 | 5.45E−25 |
| AKR1C3 | aldo-keto reductase family 1_member C3 | −13.0514 | −3.70613 | 4.31E−71 |
| ELFN1-AS1 | ELFN1 antisense RNA 1 | −13.0005 | −3.70049 | 0.001132 |
| GIMAP2 | GTPase_IMAP family member 2 | −12.977 | −3.69789 | 0.000193 |
| EPHA3 | EPH receptor A3 | −12.9631 | −3.69634 | 4.89E−09 |
| AMDHD1 | amidohydrolase domain containing 1 | −12.8293 | −3.68137 | 2.56E−06 |
| DHRS3 | dehydrogenase/reductase (SDR family) | −12.8017 | −3.67826 | 3.71E−99 |
| | member 3 | | | |
| HOTAIRM1 | HOXA transcript antisense RNA_myeloid- | −12.6644 | −3.66271 | 7.70E−17 |
| | specific 1 | | | |
| LOC643733 | caspase 4_apoptosis-related cysteine peptidase | 12.6634 | −3.66259 | 0.001868 |
| | pseudogene | | | |
| PLEKHS1 | pleckstrin homology domain | −12.6564 | −3.6618 | 0.0018 |
| | containing_family S member 1 | | | |
| ALDH3A1 | aldehyde dehydrogenase 3 family_member A1 | −12.5614 | −3.65093 | 0.001122 |
| FAM124A | family with sequence similarity 124A | −12.5491 | −3.64951 | 3.37E−13 |
| APOL4 | apolipoprotein L_4 | 12.5159 | −3.64569 | 4.34E−05 |
| LOC344887 | NmrA-like family domain containing 1 | −12.445 | −3.63749 | 8.59E−08 |
| | pseudogene | | | |
| MKX | mohawk homeobox | −12.4443 | −3.63741 | 3.59E−45 |
| GPR1 | G protein-coupled receptor 1 | −12.4127 | −3.63374 | 8.62E−69 |
| C1S | complement component 1_s subcomponent | −12.3465 | −3.62603 | 5.89E−122 |
| WBP2NL | WBP2 N-terminal like | −12.3298 | −3.62408 | 0.00032 |
| ADAMTS1 | ADAM metallopeptidase with thrombospondin | −12.2866 | −3.61901 | 1.75E−51 |
| | type 1 motif_1 | | | |
| PTPRQ | protein tyrosine phosphatase_receptor type_Q | −12.2197 | −3.61114 | 6.07E−15 |
| ADRA1D | adrenoceptor alpha 1D | −12.2063 | −3.60955 | 3.37E−33 |
| MIR4768 | microRNA 4768 | −12.1847 | −3.607 | 5.42E−07 |
| BPIFB4 | BPI fold containing family B_member 4 | −12.181 | −3.60656 | 0.002036 |
| GCNT1 | glucosaminyl (N-acetyl) transferase 1_core 2 | −12.1357 | −3.60118 | 7.82E−213 |
| THBS1 | thrombospondin 1 | −12.1223 | −3.59959 | 8.45E−26 |
| KLF15 | Kruppel-like factor 15 | −12.1204 | −3.59936 | 5.06E−08 |
| ICAM2 | intercellular adhesion molecule 2 | −12.0635 | −3.59258 | 1.67E−16 |
| LINC00264 | long intergenic non-protein coding RNA 264 | −12.0558 | −3.59166 | 0.002903 |
| HAR1B | highly accelerated region 1B (non-protein | −12.0333 | −3.58896 | 0.000378 |
| | coding) | | | |
| KRT32 | keratin 32_type I | −11.9989 | −3.58483 | 0.000268 |
| TRPA1 | transient receptor potential cation | −11.9929 | −3.58411 | 1.13E−09 |
| | channel_subfamily A_member 1 | | | |
| CACNA1C- | CACNA1C antisense RNA 1 | −11.9457 | −3.57842 | 3.11E−05 |
| AS1 | | | | |
| RXFP1 | relaxin/insulin-like family peptide receptor 1 | −11.9372 | −3.57739 | 0.00163 |
| HSPA7 | heat shock 70 kDa protein 7 (HSP70B) | −11.9275 | −3.57622 | 0.002637 |
| ZSWIM2 | zinc finger_SWIM-type containing 2 | −11.9 | −3.57289 | 5.58E−09 |
| POM121L9P | POM121 transmembrane nucleoporin-like | −11.8915 | −3.57186 | 2.42E−12 |
| | 9_pseudogene | | | |
| PLA2R1 | phospholipase A2 receptor 1_180 kDa | −11.8505 | −3.56687 | 6.96E−87 |
| LOC100506258 | uncharacterized LOC100506258 | −11.8385 | −3.56541 | 7.21E−08 |
| MIR27A | microRNA 27a | −11.8194 | −3.56308 | 0.00043 |
| XAF1 | XIAP associated factor 1 | −11.8067 | −3.56153 | 1.34E−19 |
| C21orf15 | cytochrome P450_family 4_subfamily | −11.7779 | −3.55801 | 0.00238 |
| | F_polypeptide 29_pseudogene | | | |
| FIBCD1 | fibrinogen C domain containing 1 | −11.7398 | −3.55333 | 1.37E−52 |
| TLX2 | T-cell leukemia homeobox 2 | −11.7162 | −3.55043 | 0.000158 |
| PSG2 | pregnancy specific beta-1-glycoprotein 2 | −11.7126 | −3.54999 | 0.001543 |
| PCDHGB5 | protocadherin gamma subfamily B_5 | −11.6849 | −3.54657 | 1.84E−09 |
| RNF212 | ring finger protein 212 | −11.6282 | −3.53956 | 1.92E−84 |
| HERC2P10 | hect domain and RLD 2 pseudogene 10 | −11.6193 | −3.53845 | 7.54E−07 |
| SHCBP1L | SHC SH2-domain binding protein 1-like | −11.585 | −3.53418 | 0.001762 |
| FKBP9P1 | FK506 binding protein 9 pseudogene 1 | −11.5254 | −3.52674 | 2.78E−17 |
| MAB21L3 | mab-21-like 3 (C. elegans) | −11.5082 | −3.52459 | 0.002871 |
| C9orf64 | chromosome 9 open reading frame 64 | −11.5027 | −3.5239 | 1.20E−22 |
| TDRD12 | tudor domain containing 12 | −11.4976 | −3.52326 | 0.003909 |
| FXYD3 | FXYD domain containing ion transport | −11.4449 | −3.51663 | 0.002241 |
| | regulator 3 | | | |
| PCDHB15 | protocadherin beta 15 | −11.4199 | −3.51348 | 4.29E−16 |
| HTATSF1P2 | HIV-1 Tat specific factor 1 pseudogene 2 | −11.4056 | −3.51167 | 1.54E−18 |
| KRTAP1-3 | keratin associated protein 1-3 | −11.405 | −3.5116 | 0.000615 |
| ESR1 | estrogen receptor 1 | −11.4016 | −3.51116 | 1.74E−09 |
| TDRD6 | tudor domain containing 6 | −11.3952 | −3.51035 | 5.09E−06 |
| SLC4A4 | solute carrier family 4 (sodium bicarbonate | −11.3712 | −3.50731 | 6.76E−68 |
| | cotransporter)_member 4 | | | |
| IL26 | interleukin 26 | −11.2376 | −3.49026 | 1.05E−06 |
| LIN7A | lin-7 homolog A (C. elegans) | −11.1763 | −3.48237 | 1.60E−19 |
| C2orf88 | chromosome 2 open reading frame 88 | −11.153 | −3.47936 | 6.65E−36 |
| PRRX2 | paired related homeobox 2 | −11.1516 | −3.47918 | 4.79E−30 |
| CASC1 | cancer susceptibility candidate 1 | −11.132 | −3.47664 | 8.72E−06 |
| HTR6 | 5-hydroxytryptamine (serotonin) receptor 6_G | −11.1213 | −3.47525 | 9.95E−05 |
| | protein-coupled | | | |
| STAT4 | signal transducer and activator of transcription | −11.0841 | −3.47042 | 3.13E−15 |
| | 4 | | | |
| MEIS3P1 | Meis homeobox 3 pseudogene 1 | −11.0567 | −3.46685 | 5.16E−15 |
| PCAT5 | prostate cancer associated transcript 5 (non- | −11.0216 | −3.46226 | 0.003735 |
| | protein coding) | | | |
| LEP | leptin | −10.9642 | −3.45473 | 0.002439 |
| SETBP1 | SET binding protein 1 | −10.9298 | −3.4502 | 1.13E−13 |
| CEACAM22P | carcinoembryonic antigen-related cell adhesion | −10.913 | −3.44797 | 0.003652 |
| | molecule 22_pseudogene | | | |
| C4orf32 | chromosome 4 open reading frame 32 | −10.8864 | −3.44445 | 2.30E−28 |
| LINC00943 | long intergenic non-protein coding RNA 943 | −10.8536 | −3.4401 | 0.004493 |
| ZNF541 | zinc finger protein 541 | −10.8222 | −3.43592 | 0.001083 |
| CC2D2B | coiled-coil and C2 domain containing 2B | −10.8072 | −3.43392 | 0.002733 |
| LOC340113 | uncharacterized LOC340113 | −10.7923 | −3.43193 | 0.003322 |
| RAB3IL1 | RAB3A interacting protein (rabin3)-like 1 | −10.7393 | −3.42483 | 1.25E−78 |
| LEPR | leptin receptor | −10.7172 | −3.42186 | 3.94E−24 |
| CACNA1C | calcium channel_voltage-dependent_L | −10.6935 | −3.41866 | 8.21E−37 |
| | type_alpha 1C subunit | | | |
| LMO7-AS1 | LMO7 antisense RNA 1 | −10.6495 | −3.41271 | 8.67E−21 |
| C1R | complement component 1_r subcomponent | −10.6162 | −3.4082 | 3.20E−139 |
| SLC9A9 | solute carrier family 9_subfamily A | −10.6078 | −3.40705 | 3.19E−37 |
| | (NHE9_cation proton antiporter 9)_member 9 | | | |
| LOC102724927 | uncharacterized LOC102724927 | −10.5947 | −3.40527 | 1.19E−20 |
| DPT | dermatopontin | −10.5809 | −3.40339 | 0.005268 |
| EMP1 | epithelial membrane protein 1 | −10.5387 | −3.39763 | 3.56E−26 |
| ZNF676 | zinc finger protein 676 | −10.5336 | −3.39693 | 0.00396 |
| LIMCH1 | LIM and calponin homology domains 1 | −10.5325 | −3.39678 | 3.84E−19 |
| PLXNA4 | plexin A4 | −10.5205 | −3.39513 | 1.94E−23 |
| MT1M | metallothionein 1M | −10.5182 | −3.39481 | 3.35E−15 |
| TENM2 | teneurin transmembrane protein 2 | −10.5068 | −3.39325 | 2.22E−96 |
| WISP1 | WNT1 inducible signaling pathway protein 1 | −10.468 | −3.38792 | 2.62E−25 |
| LOC391322 | D-dopachrome tautomerase-like | −10.3516 | −3.37178 | 9.00E−12 |
| CMAHP | cytidine monophospho-N-acetylneuraminic | −10.3496 | −3.37151 | 5.26E−27 |
| | acid hydroxylase_pseudogene | | | |
| MIR92B | microRNA 92b | −10.3491 | −3.37144 | 0.004725 |
| IL7 | interleukin 7 | −10.3346 | −3.36941 | 7.43E−15 |
| KRT33B | keratin 33B_type I | −10.3158 | −3.36678 | 3.78E−29 |
| FAM109B | family with sequence similarity 109_member | −10.2915 | −3.36338 | 1.99E−239 |
| | B | | | |
| TGM5 | transglutaminase 5 | −10.2562 | −3.35843 | 0.006672 |
| PAX8 | paired box 8 | −10.2492 | −3.35744 | 0.000304 |
| SOCS2 | suppressor of cytokine signaling 2 | −10.2112 | −3.35208 | 2.28E−77 |
| MEGF6 | multiple EGF-like-domains 6 | −10.2066 | −3.35143 | 5.05E−52 |
| ALOX15P1 | arachidonate 15-lipoxygenase pseudogene 1 | −10.2064 | −3.3514 | 0.004495 |
| LINC00982 | long intergenic non-protein coding RNA 982 | −10.1965 | −3.35 | 3.29E−20 |
| ZNF560 | zinc finger protein 560 | −10.1806 | −3.34775 | 2.83E−07 |
| FOS | FBJ murine osteosarcoma viral oncogene | −10.1784 | −3.34744 | 4.35E−15 |
| | homolog | | | |
| ASPN | asporin | −10.1769 | −3.34723 | 3.42E−05 |
| CNTNAP2 | contactin associated protein-like 2 | −10.1411 | −3.34214 | 4.27E−07 |
| ESM1 | endothelial cell-specific molecule 1 | −10.1294 | −3.34047 | 8.19E−14 |
| CTSW | cathepsin W | −10.1277 | −3.34023 | 0.000387 |
| NFIX | nuclear factor I/X (CCAAT-binding | −9.99031 | −3.32053 | 2.37E−35 |
| | transcription factor) | | | |
| GCKR | glucokinase (hexokinase 4) regulator | −9.97447 | −3.31824 | 1.31E−20 |
| HOXC11 | homeobox C11 | −9.96673 | −3.31712 | 8.15E−41 |
| B4GALNT1 | beta-1_4-N-acetyl-galactosaminyl transferase | −9.96445 | −3.31679 | 2.03E−47 |
| | 1 | | | |
| LRRC2-AS1 | LRRC2 antisense RNA 1 | −9.9483 | −3.31445 | 0.004436 |
| ALDH1L2 | aldehyde dehydrogenase 1 family_member L2 | −9.92626 | −3.31125 | 3.37E−31 |
| DOCK9-AS2 | DOCK9 antisense RNA 2 (head to head) | −9.92564 | −3.31116 | 4.99E−08 |
| ROCK1P1 | Rho-associated_coiled-coil containing protein | −9.8439 | −3.29923 | 0.000897 |
| | kinase 1 pseudogene 1 | | | |
| LTC4S | leukotriene C4 synthase | −9.83353 | −3.29771 | 4.10E−27 |
| HOXA7 | homeobox A7 | −9.7738 | −3.28892 | 5.73E−17 |
| PCDHGA8 | protocadherin gamma subfamily A_8 | −9.76602 | −3.28777 | 3.68E−30 |
| TECTB | tectorin beta | −9.74931 | −3.2853 | 0.006826 |
| LINC00965 | long intergenic non-protein coding RNA 965 | −9.72515 | −3.28172 | 0.005153 |
| S100P | S100 calcium binding protein P | −9.7143 | −3.28011 | 0.002216 |
| TTTY10 | testis-specific transcript_Y-linked 10 (non- | −9.70481 | −3.2787 | 0.008667 |
| | protein coding) | | | |
| ALDH3B1 | aldehyde dehydrogenase | −9.68338 | −3.27551 | 8.63E−125 |
| | 3 family_member B1 | | | |
| C1orf158 | chromosome 1 open reading frame 158 | −9.65944 | −3.27194 | 0.006341 |
| LOC101927755 | uncharacterized LOC101927755 | −9.65536 | −3.27133 | 3.78E−06 |
| MSR1 | macrophage scavenger receptor 1 | −9.65375 | −3.27109 | 2.50E−11 |
| TNFSF11 | tumor necrosis factor (ligand) | −9.64439 | −3.26969 | 0.004874 |
| | superfamily_member 11 | | | |
| C5orf38 | chromosome 5 open reading frame 38 | −9.64305 | −3.26949 | 2.16E−35 |
| CFI | complement factor I | −9.63697 | −3.26858 | 2.03E−37 |
| TCF7 | transcription factor 7 (T-cell specific_HMG- | −9.60729 | −3.26413 | 4.99E−36 |
| | box) | | | |
| CD80 | CD80 molecule | −9.60576 | −3.2639 | 0.004995 |
| MIR6071 | microRNA 6071 | −9.6027 | −3.26344 | 0.007925 |
| LCN1 | lipocalin 1 | −9.59651 | −3.26251 | 2.06E−05 |
| IL1R2 | interleukin 1 receptor_type II | −9.58634 | −3.26098 | 0.000578 |
| LOC100506895 | uncharacterized LOC100506895 | −9.56291 | −3.25745 | 1.66E−05 |
| A2ML1 | alpha-2-macroglobulin-like 1 | −9.54662 | −3.25499 | 0.0002 |
| AFF2 | AF4/FMR2 family_member 2 | −9.53809 | −3.2537 | 4.80E−45 |
| NKG7 | natural killer cell granule protein 7 | −9.51933 | −3.25086 | 0.002237 |
| SIGLEC10 | sialic acid binding Ig-like lectin 10 | −9.46584 | −3.24273 | 0.002419 |
| TRIM4 | tripartite motif containing 4 | −9.44283 | −3.23922 | 3.32E−58 |
| ZG16B | zymogen granule protein 16B | −9.43708 | −3.23834 | 8.22E−07 |
| CCDC158 | coiled-coil domain containing 158 | −9.40514 | −3.23345 | 3.13E−10 |
| FGL2 | fibrinogen-like 2 | −9.40299 | −3.23312 | 0.000861 |
| LOC101927688 | NA | −9.39908 | −3.23252 | 9.42E−05 |
| INHBB | inhibin_beta B | −9.38645 | −3.23058 | 8.02E−123 |
| HOXA10 | homeobox A10 | −9.35158 | −3.22521 | 1.51E−123 |
| FHAD1 | forkhead-associated (FHA) phosphopeptide | −9.33345 | −3.22241 | 1.89E−06 |
| | binding domain 1 | | | |
| OSR2 | odd-skipped related transciption factor 2 | −9.30935 | −3.21868 | 1.36E−05 |
| SNORD114-26 | small nucleolar RNA_C/D box 114-26 | −9.27939 | −3.21403 | 0.008533 |
| NKX6-1 | NK6 homeobox 1 | −9.26352 | −3.21156 | 1.76E−15 |
| DNER | delta/notch-like EGF repeat containing | −9.25472 | −3.21019 | 2.07E−06 |
| LDHAL6B | lactate dehydrogenase A-like 6B | −9.24985 | −3.20943 | 0.001253 |
| C11orf86 | chromosome 11 open reading frame 86 | −9.24556 | −3.20876 | 5.32E−05 |
| VSTM4 | V-set and transmembrane domain containing 4 | −9.21485 | −3.20396 | 2.42E−29 |
| HOXA3 | homeobox A3 | −9.19985 | −3.20161 | 1.76E−26 |
| HOXC-AS3 | HOXC cluster antisense RNA 3 | −9.18303 | −3.19897 | 4.60E−08 |
| NPY6R | neuropeptide Y receptor Y6 (pseudogene) | −9.17883 | −3.19831 | 0.009703 |
| HSD11B1 | hydroxysteroid (11-beta) dehydrogenase 1 | −9.17323 | −3.19743 | 0.008005 |
| LINC01220 | long intergenic non-protein coding RNA 1220 | −9.16738 | −3.19651 | 0.001771 |
| MB21D1 | Mab-21 domain containing 1 | −9.16484 | −3.19611 | 1.32E−26 |
| RNF43 | ring finger protein 43 | −9.14802 | −3.19346 | 0.001612 |
| HEYL | hes-related family bHLH transcription factor | −9.14321 | −3.1927 | 0.000477 |
| | with YRPW motif-like | | | |
| TNIP3 | TNFAIP3 interacting protein 3 | −9.14295 | −3.19266 | 1.78E−12 |
| SMCR9 | NA | −9.12902 | −3.19046 | 0.009476 |
| SNORD114-1 | small nucleolar RNA_C/D box 114-1 | −9.11006 | −3.18746 | 0.007346 |
| CCRL2 | chemokine (C-C motif) receptor-like 2 | −9.04819 | −3.17763 | 0.005581 |
| GOLGA8O | golgin A8 family_member O | −9.00421 | −3.1706 | 4.43E−05 |
| MIR615 | microRNA 615 | −8.99835 | −3.16966 | 0.009703 |
| KLF17 | Kruppel-like factor 17 | −8.98289 | −3.16718 | 3.70E−05 |
| BST1 | bone marrow stromal cell antigen 1 | −8.96697 | −3.16462 | 4.52E−64 |
| MIR199A1 | microRNA 199a-1 | −8.87877 | −3.15036 | 0.000581 |
| SERP2 | stress-associated endoplasmic reticulum | −8.87533 | −3.1498 | 1.63E−47 |
| | protein family member 2 | | | |
| S100B | S100 calcium binding protein B | −8.87514 | −3.14977 | 9.46E−08 |
| ZNF726 | zinc finger protein 726 | −8.84578 | −3.14499 | 8.39E−07 |
| COL16A1 | collagen_type XVI_alpha 1 | −8.83647 | −3.14347 | 1.43E−59 |
| TMEM30B | transmembrane protein 30B | −8.83133 | −3.14263 | 1.45E−07 |
| FLJ46906 | uncharacterized LOC441172 | −8.81866 | −3.14056 | 1.58E−13 |
| SCRT1 | scratch family zinc finger 1 | −8.77945 | −3.13413 | 0.007849 |
| GDAP1L1 | ganglioside induced differentiation associated | −8.77622 | −3.1336 | 0.000269 |
| | protein 1-like 1 | | | |
| TRPM2 | transient receptor potential cation | −8.76959 | −3.13251 | 4.32E−08 |
| | channel_subfamily M_member 2 | | | |
| CSMD1 | CUB and Sushi multiple domains 1 | −8.74592 | −3.12861 | 0.00292 |
| FTCDNL1 | formiminotransferase cyclodeaminase N- | −8.73253 | −3.1264 | 4.10E−05 |
| | terminal like | | | |
| RIMS1 | regulating synaptic membrane exocytosis 1 | −8.72987 | −3.12596 | 5.39E−29 |
| MIR409 | microRNA 409 | −8.70721 | −3.12221 | 0.008342 |
| RCN3 | reticulocalbin 3_EF-hand calcium binding | −8.69955 | −3.12094 | 3.55E−43 |
| | domain | | | |
| LOC101927354 | uncharacterized LOC101927354 | −8.68551 | −3.11861 | 2.09E−06 |
| PLA2G16 | phospholipase A2_group XVI | −8.68226 | −3.11807 | 8.21E−118 |
| SLC1A3 | solute carrier family 1 (glial high affinity | −8.64838 | −3.11243 | 8.42E−16 |
| | glutamate transporter)_member 3 | | | |
| CARD16 | caspase recruitment domain family_member | −8.61225 | −3.10639 | 2.92E−14 |
| | 16 | | | |
| LOC101927667 | NA | −8.61094 | −3.10617 | 1.92E−05 |
| DAPK1 | death-associated protein kinase 1 | −8.59746 | −3.10391 | 1.87E−56 |
| ANGPT1 | angiopoietin 1 | −8.57235 | −3.09969 | 2.50E−11 |
| ACOX2 | acyl-CoA oxidase 2_branched chain | −8.56189 | −3.09793 | 4.59E−28 |
| GHDC | GH3 domain containing | −8.55483 | −3.09674 | 3.85E−76 |
| IGFBP1 | insulin-like growth factor binding protein 1 | −8.53103 | −3.09272 | 0.004458 |
| PDE7B | phosphodiesterase 7B | −8.52488 | −3.09168 | 3.28E−45 |
| MACROD2 | MACRO domain containing 2 | −8.51166 | −3.08944 | 7.31E−06 |
| RSPO2 | R-spondin 2 | −8.50788 | −3.0888 | 0.009007 |
| KCNJ9 | potassium channel_inwardly rectifying | −8.42934 | −3.07542 | 0.003859 |
| | subfamily J_member 9 | | | |
| LOC101059948 | uncharacterized LOC101059948 | −8.42554 | −3.07477 | 8.21E−06 |
| GPR68 | G protein-coupled receptor 68 | −8.42543 | −3.07475 | 5.26E−24 |
| SOX9-AS1 | SOX9 antisense RNA 1 | −8.3768 | −3.0664 | 0.001328 |
| RDH5 | retinol dehydrogenase 5 (11-cis/9-cis) | −8.36862 | −3.06499 | 9.26E−19 |
| NLRP3 | NLR family_pyrin domain containing 3 | −8.32102 | −3.05676 | 1.57E−20 |
| SLC22A3 | solute carrier family 22 (organic cation | −8.31762 | −3.05617 | 7.45E−15 |
| | transporter)_member 3 | | | |
| G0S2 | G0/G1 switch 2 | −8.30817 | −3.05453 | 4.71E−17 |
| LOC100505739 | NA | −8.29372 | −3.05202 | 0.00648 |
| C21orf67 | long intergenic non-protein coding RNA 1547 | −8.28844 | −3.0511 | 1.50E−39 |
| CHST15 | carbohydrate (N-acetylgalactosamine 4-sulfate | −8.23107 | −3.04108 | 4.79E−40 |
| | 6-O) sulfotransferase 15 | | | |
| HOXD1 | homeobox D1 | −8.22674 | −3.04032 | 0.005994 |
| HOXA2 | homeobox A2 | −8.20418 | −3.03636 | 3.98E−13 |
| TRIB3 | tribbles pseudokinase 3 | −8.18805 | −3.03352 | 3.54E−51 |
| LOC100129722 | NA | −8.18147 | −3.03236 | 1.67E−05 |
| CCIN | calicin | −8.17223 | −3.03073 | 1.07E−17 |
| ITGB8 | integrin_beta 8 | −8.16131 | −3.0288 | 8.36E−29 |
| HIST2H2BA | histone cluster 2_H2ba (pseudogene) | −8.13657 | −3.02442 | 8.34E−11 |
| PIWIL2 | piwi-like RNA-mediated gene silencing 2 | −8.13177 | −3.02357 | 1.37E−05 |
| ID4 | inhibitor of DNA binding 4_dominant | −8.11865 | −3.02124 | 7.49E−94 |
| | negative helix-loop-helix protein | | | |
| EVI2B | ecotropic viral integration site 2B | −8.11263 | −3.02017 | 2.32E−16 |
| LOC375196 | uncharacterized LOC375196 | −8.07711 | −3.01384 | 3.53E−06 |
| WEE2-AS1 | WEE2 antisense RNA 1 | −8.07107 | −3.01276 | 2.72E−12 |
| GYPE | glycophorin E (MNS blood group) | −8.04454 | −3.00801 | 0.006375 |
| OXT | oxytocin/neurophysin I prepropeptide | −8.04393 | −3.0079 | 0.001064 |
| LOC102724550 | NA | −8.03317 | −3.00597 | 1.79E−11 |
| FAM87A | family with sequence similarity 87_member A | −8.0081 | −3.00146 | 0.004949 |
| VLDLR-AS1 | VLDLR antisense RNA 1 | −7.96365 | −2.99343 | 8.01E−16 |
| NECAB2 | N-terminal EF-hand calcium binding protein 2 | −7.9535 | −2.99159 | 1.90E−11 |
| ACSS3 | acyl-CoA synthetase short-chain family | −7.94006 | −2.98915 | 1.83E−60 |
| | member 3 | | | |
| LOC284798 | uncharacterized LOC284798 | −7.92994 | −2.98731 | 0.006819 |
| MYO18B | myosin XVIIIB | −7.92143 | −2.98576 | 0.004941 |
| UBE2QL1 | ubiquitin-conjugating enzyme E2Q family-like | −7.91692 | −2.98494 | 2.09E−05 |
| | 1 | | | |
| MFSD7 | major facilitator superfamily domain | −7.85287 | −2.97322 | 9.43E−57 |
| | containing 7 | | | |
| PNMA2 | paraneoplastic Ma antigen 2 | −7.81074 | −2.96546 | 8.09E−11 |
| FXYD1 | FXYD domain containing ion transport | −7.79906 | −2.9633 | 1.83E−11 |
| | regulator 1 | | | |
| PGF | placental growth factor | −7.75142 | −2.95446 | 1.58E−176 |
| RAD21-AS1 | RAD21 antisense RNA 1 | −7.75109 | −2.9544 | 0.001458 |
| ZFP57 | ZFP57 zinc finger protein | −7.72037 | −2.94867 | 0.002055 |
| CRNDE | colorectal neoplasia differentially expressed | −7.70092 | −2.94503 | 3.84E−139 |
| | (non-protein coding) | | | |
| BAALC | brain and acute leukemia_cytoplasmic | −7.68764 | −2.94254 | 2.92E−09 |
| LURAP1L | leucine rich adaptor protein 1-like | −7.65939 | −2.93723 | 1.58E−119 |
| NOV | nephroblastoma overexpressed | −7.65419 | −2.93625 | 3.00E−29 |
| CALHM2 | calcium homeostasis modulator 2 | −7.65409 | −2.93623 | 2.60E−57 |
| TEC | tec protein tyrosine kinase | −7.64428 | −2.93438 | 2.13E−10 |
| LOC101928036 | NA | −7.62586 | −2.9309 | 3.39E−06 |
| MACC1 | metastasis associated in colon cancer 1 | −7.61276 | −2.92842 | 0.000151 |
| FGR | FGR | −7.51472 | −2.90972 | 0.007795 |
| | proto-oncogene_Src family tyrosine | | | |
| | kinase | | | |
| GPR85 | G protein-coupled receptor 85 | −7.50577 | −2.908 | 3.58E−33 |
| MIR24-2 | microRNA 24-2 | −7.48909 | −2.90479 | 6.35E−05 |
| HTRA1 | HtrA serine peptidase 1 | −7.43493 | −2.89432 | 3.38E−46 |
| CD97 | adhesion G protein-coupled receptor E5 | −7.39628 | −2.8868 | 5.45E−18 |
| OXCT1-AS1 | OXCT1 antisense RNA 1 | −7.39459 | −2.88647 | 0.000781 |
| LOC101928891 | uncharacterized LOC101928891 | −7.38977 | −2.88553 | 6.18E−09 |
| SVILP1 | supervillin pseudogene 1 | −7.37176 | −2.88201 | 0.002022 |
| LINC00619 | long intergenic non-protein coding RNA 619 | −7.36349 | −2.88039 | 1.40E−11 |
| PITX1 | paired-like homeodomain 1 | −7.33329 | −2.87446 | 1.60E−40 |
| DDIT4 | DNA-damage-inducible transcript 4 | −7.32445 | −2.87272 | 2.85E−30 |
| DAPK2 | death-associated protein kinase 2 | −7.3142 | −2.8707 | 1.43E−19 |
| PCDHGB2 | protocadherin gamma subfamily B_2 | −7.27169 | −2.86229 | 2.78E−16 |
| PDE2A | phosphodiesterase 2A cGMP-stimulated | −7.25291 | −2.85856 | 3.93E−05 |
| SLC38A5 | solute carrier family 38_member 5 | −7.21735 | −2.85147 | 1.83E−60 |
| TRPC6 | transient receptor potential cation | −7.20945 | −2.84989 | 7.86E−05 |
| | channel_subfamily C_member 6 | | | |
| ITGA10 | integrin_alpha 10 | −7.19378 | −2.84675 | 1.86E−15 |
| CXCL3 | chemokine (C-X-C motif) ligand 3 | −7.19218 | −2.84643 | 5.23E−06 |
| CFD | complement factor D (adipsin) | −7.19049 | −2.84609 | 3.15E−27 |
| FAM78B | family with sequence similarity 78_member B | −7.18386 | −2.84476 | 1.29E−05 |
| C2orf73 | chromosome 2 open reading frame 73 | −7.17794 | −2.84357 | 0.003713 |
| ITGA7 | integrin_alpha 7 | −7.17232 | −2.84244 | 5.49E−11 |
| VDR | vitamin D (1_25-dihydroxyvitamin D3) | −7.15926 | −2.83981 | 4.27E−46 |
| | receptor | | | |
| LOC100506188 | uncharacterized LOC100506188 | −7.15311 | −2.83857 | 5.64E−11 |
| LOC100240734 | uncharacterized LOC100240734 | −7.12801 | −2.8335 | 0.004573 |
| PRG4 | proteoglycan 4 | −7.11523 | −2.83091 | 7.94E−17 |
| LOC102723769 | uncharacterized LOC102723769 | −7.11064 | −2.82998 | 0.008421 |
| SLC30A2 | solute carrier family 30 (zinc | −7.10237 | −2.8283 | 0.000171 |
| | transporter)_member 2 | | | |
| MISP | mitotic spindle positioning | −7.10183 | −2.82819 | 7.07E−08 |
| MTSS1 | metastasis suppressor 1 | −7.09096 | −2.82598 | 1.40E−16 |
| FAM178B | family with sequence similarity 178_member | −7.09027 | −2.82584 | 6.79E−05 |
| | B | | | |
| C15orf59 | chromosome 15 open reading frame 59 | −7.08467 | −2.8247 | 1.37E−32 |
| FAM167A | family with sequence similarity 167_member | −7.08197 | −2.82415 | 2.71E−20 |
| | A | | | |
| LOC101929234 | uncharacterized LOC101929234 | −7.06216 | −2.82011 | 3.24E−07 |
| CSF1R | colony stimulating factor 1 receptor | −7.04705 | −2.81702 | 5.45E−12 |
| PRSS12 | protease_serine_12 (neurotrypsin_motopsin) | −7.04642 | −2.81689 | 4.76E−14 |
| HCG4B | HLA complex group 4B (non-protein coding) | −7.03246 | −2.81403 | 0.000191 |
| CYB561 | cytochrome b561 | −7.01533 | −2.81051 | 5.12E−66 |
| TMEM150C | transmembrane protein 150C | −6.98447 | −2.80415 | 2.34E−33 |
| LY75 | lymphocyte antigen 75 | −6.98326 | −2.8039 | 0.003764 |
| VCAM1 | vascular cell adhesion molecule 1 | −6.97542 | −2.80228 | 6.95E−17 |
| ZNF667-AS1 | ZNF667 antisense RNA 1 (head to head) | −6.9484 | −2.79668 | 1.23E−19 |
| ALPK1 | alpha-kinase 1 | −6.94238 | −2.79543 | 6.99E−35 |
| ZNF354C | zinc finger protein 354C | −6.93824 | −2.79457 | 2.95E−06 |
| ZNF396 | zinc finger protein 396 | −6.93516 | −2.79393 | 3.40E−07 |
| NDRG1 | N-myc downstream regulated 1 | −6.93439 | −2.79377 | 5.51E−30 |
| ZNF829 | zinc finger protein 829 | −6.92479 | −2.79177 | 2.09E−50 |
| C10orf11 | chromosome 10 open reading frame 11 | −6.92354 | −2.79151 | 9.83E−19 |
| KRT31 | keratin 31_type I | −6.92349 | −2.7915 | 0.000395 |
| NTRK1 | neurotrophic tyrosine kinase_receptor_type 1 | −6.91726 | −2.7902 | 0.00105 |
| PRDM6 | PR domain containing 6 | −6.89452 | −2.78545 | 1.36E−05 |
| KCNJ8 | potassium channel_inwardly rectifying | −6.89323 | −2.78518 | 1.33E−44 |
| | subfamily J_member 8 | | | |
| FZD5 | frizzled class receptor 5 | −6.88306 | −2.78305 | 1.81E−09 |
| KLF9 | Kruppel-like factor 9 | −6.87905 | −2.78221 | 1.75E−17 |
| GGT5 | gamma-glutamyltransferase 5 | −6.87896 | −2.78219 | 1.66E−19 |
| LOC115110 | uncharacterized LOC115110 | −6.8771 | −2.7818 | 0.002611 |
| SCRG1 | stimulator of chondrogenesis 1 | −6.86286 | −2.77881 | 2.32E−19 |
| OTUD7A | OTU deubiquitinase 7A | −6.86253 | −2.77874 | 0.001651 |
| C15orf65 | chromosome 15 open reading frame 65 | −6.85963 | −2.77813 | 4.91E−26 |
| AGBL2 | ATP/GTP binding protein-like 2 | −6.85255 | −2.77664 | 1.04E−09 |
| NR4A3 | nuclear receptor subfamily 4_group | −6.83504 | −2.77295 | 6.82E−11 |
| | A_member 3 | | | |
| FOXC1 | forkhead box C1 | −6.78684 | −2.76274 | 6.06E−51 |
| VCAN | versican | −6.7773 | −2.76071 | 6.67E−20 |
| MILR1 | mast cell immunoglobulin-like receptor 1 | −6.74767 | −2.75439 | 1.29E−07 |
| KLF2 | Kruppel-like factor 2 | −6.74019 | −2.75279 | 2.40E−150 |
| ESPNL | espin-like | −6.73748 | −2.75221 | 0.000167 |
| JHDM1D-AS1 | JHDM1D antisense RNA 1 (head to head) | −6.73137 | −2.7509 | 5.34E−41 |
| CFH | complement factor H | −6.70659 | −2.74558 | 9.08E−17 |
| MIR4664 | microRNA 4664 | −6.70241 | −2.74468 | 0.002155 |
| SLC1A1 | solute carrier family 1 (neuronal/epithelial high | −6.69944 | −2.74404 | 2.47E−45 |
| | affinity glutamate transporter_system | | | |
| | Xag)_member 1 | | | |
| HOXA-AS3 | HOXA cluster antisense RNA 3 | −6.67751 | −2.73931 | 8.73E−21 |
| RADIL | Ras association and DIL domains | −6.66867 | −2.7374 | 2.59E−11 |
| HOXA4 | homeobox A4 | −6.66258 | −2.73608 | 6.22E−18 |
| NAT2 | N-acetyltransferase 2 (arylamine N- | −6.64993 | −2.73334 | 0.001906 |
| | acetyltransferase) | | | |
| LINC00936 | long intergenic non-protein coding RNA 936 | −6.6138 | −2.72548 | 1.61E−28 |
| LINC00595 | long intergenic non-protein coding RNA 595 | −6.60954 | −2.72455 | 7.72E−07 |
| COLEC12 | collectin sub-family member 12 | −6.60904 | −2.72444 | 1.14E−34 |
| CST6 | cystatin E/M | −6.59403 | −2.72116 | 8.41E−10 |
| SMOC1 | SPARC related modular calcium binding 1 | −6.58603 | −2.71941 | 1.06E−12 |
| BEX1 | brain expressed_X-linked 1 | −6.55884 | −2.71344 | 1.73E−78 |
| ADM2 | adrenomedullin 2 | −6.55079 | −2.71167 | 2.80E−43 |
| NXPH4 | neurexophilin 4 | −6.54045 | −2.70939 | 3.69E−35 |
| IL1RL2 | interleukin 1 receptor-like 2 | −6.52881 | −2.70682 | 5.90E−11 |
| LOC101060542 | uncharacterized LOC101060542 | −6.52261 | −2.70545 | 0.001517 |
| ENG | endoglin | −6.51701 | −2.70421 | 3.07E−110 |
| RNLS | renalase_FAD-dependent amine oxidase | −6.49167 | −2.69859 | 3.94E−23 |
| OLFML3 | olfactomedin-like 3 | −6.48659 | −2.69746 | 1.13E−28 |
| KLHDC7B | kelch domain containing 7B | −6.47549 | −2.69499 | 3.22E−12 |
| SLC38A3 | solute carrier family 38_member 3 | −6.47092 | −2.69397 | 1.15E−10 |
| CRISPLD2 | cysteine-rich secretory protein LCCL domain | −6.43576 | −2.68611 | 5.56E−22 |
| | containing 2 | | | |
| DUSP2 | dual specificity phosphatase 2 | −6.41158 | −2.68068 | 1.10E−41 |
| PER3 | period circadian clock 3 | −6.39746 | −2.6775 | 3.51E−25 |
| TYMP | thymidine phosphorylase | −6.38732 | −2.67521 | 3.10E−35 |
| GSTO2 | glutathione S-transferase omega 2 | −6.38254 | −2.67413 | 6.70E−56 |
| LOC730102 | quinone oxidoreductase-like protein 2 | −6.37736 | −2.67296 | 2.98E−80 |
| | pseudogene | | | |
| STAC2 | SH3 and cysteine rich domain 2 | −6.37007 | −2.67131 | 4.20E−16 |
| PMP22 | peripheral myelin protein 22 | −6.35777 | −2.66852 | 4.22E−47 |
| CCR7 | chemokine (C-C motif) receptor 7 | −6.35495 | −2.66788 | 4.47E−19 |
| HECW1 | HECT_C2 and WW domain containing E3 | −6.33265 | −2.66281 | 4.22E−09 |
| | ubiquitin protein ligase 1 | | | |
| PKP1 | plakophilin 1 | −6.32077 | −2.6601 | 6.21E−08 |
| BICC1 | BicC family RNA binding protein 1 | −6.30488 | −2.65647 | 3.68E−11 |
| C11orf87 | chromosome 11 open reading frame 87 | −6.27715 | −2.65011 | 1.73E−10 |
| ANKH | ANKH inorganic pyrophosphate transport | −6.27311 | −2.64918 | 2.02E−12 |
| | regulator | | | |
| CCPG1 | cell cycle progression 1 | −6.25517 | −2.64505 | 4.34E−25 |
| NIM1K | NIMI serine/threonine protein kinase | −6.23474 | −2.64033 | 1.29E−12 |
| ISL2 | ISL LIM homeobox 2 | −6.23427 | −2.64022 | 3.68E−11 |
| TLR3 | toll-like receptor 3 | −6.21748 | −2.63633 | 5.43E−09 |
| C2 | complement component 2 | −6.20031 | −2.63234 | 2.07E−11 |
| ERAP2 | endoplasmic reticulum aminopeptidase 2 | −6.19296 | −2.63063 | 0.001254 |
| ANKRD2 | ankyrin repeat domain 2 (stretch responsive | −6.18177 | −2.62802 | 1.59E−05 |
| | muscle) | | | |
| EPB41L4B | erythrocyte membrane protein band 4.1 like 4B | −6.18018 | −2.62765 | 9.12E−16 |
| WFDC1 | WAP four-disulfide core domain 1 | −6.17248 | −2.62585 | 1.42E−09 |
| PCK2 | phosphoenolpyruvate carboxykinase 2 | −6.16803 | −2.62481 | 3.91E−40 |
| | (mitochondrial) | | | |
| ENPP1 | ectonucleotide | −6.15146 | −2.62093 | 2.52E−13 |
| | pyrophosphatase/phosphodiesterase 1 | | | |
| PRDM1 | PR domain containing 1_with ZNF domain | −6.14605 | −2.61966 | 1.49E−53 |
| FAM149A | family with sequence similarity 149_member | −6.14052 | −2.61836 | 1.20E−10 |
| | A | | | |
| MIR452 | microRNA 452 | −6.09565 | −2.60778 | 0.000336 |
| SLC22A23 | solute carrier family 22_member 23 | −6.09117 | −2.60672 | 2.51E−12 |
| LY6K | lymphocyte antigen 6 complex_locus K | −6.0611 | −2.59958 | 1.18E−06 |
| CLIC3 | chloride intracellular channel 3 | −6.05757 | −2.59874 | 3.33E−15 |
| RCAN2 | regulator of calcineurin 2 | −6.05401 | −2.59789 | 3.03E−11 |
| BEST1 | bestrophin 1 | −6.04964 | −2.59685 | 1.30E−32 |
| FRK | fyn-related Src family tyrosine kinase | −6.03846 | −2.59418 | 0.002353 |
| CEBPA | CCAAT/enhancer binding protein | −6.03373 | −2.59305 | 3.36E−13 |
| | (C/EBP)_alpha | | | |
| MROH9 | maestro heat-like repeat family member 9 | −6.01986 | −2.58973 | 0.000743 |
| RRN3P2 | RRN3 homolog_RNA polymerase I | −6.01819 | −2.58933 | 2.73E−09 |
| | transcription factor pseudogene 2 | | | |
| CASC2 | cancer susceptibility candidate 2 (non-protein | −6.01098 | −2.5876 | 5.05E−20 |
| | coding) | | | |
| TPD52L1 | tumor protein D52-like 1 | −5.99812 | −2.58451 | 5.88E−10 |
| C5orf49 | chromosome 5 open reading frame 49 | −5.98948 | −2.58243 | 2.76E−12 |
| SLC16A4 | solute carrier family 16_member 4 | −5.97807 | −2.57968 | 4.74E−35 |
| ACTC1 | actin alpha cardiac muscle 1 | −5.9597 | −2.57524 | 4.39E−07 |
| ZMYND12 | zinc finger_MYND-type containing 12 | −5.95437 | −2.57395 | 3.45E−05 |
| TEX41 | testis expressed 41 (non-protein coding) | −5.94699 | −2.57216 | 0.003728 |
| ALPK2 | alpha-kinase 2 | −5.93591 | −2.56947 | 6.22E−18 |
| TIMP4 | TIMP metallopeptidase inhibitor 4 | −5.9332 | −2.56881 | 1.17E−06 |
| VEGFC | vascular endothelial growth factor C | −5.92954 | −2.56792 | 2.29E−109 |
| SNX29P2 | sorting nexin 29 pseudogene 2 | −5.92732 | −2.56738 | 0.000105 |
| DOK1 | docking protein 1_62 kDa (downstream of | −5.91866 | −2.56527 | 1.07E−31 |
| | tyrosine kinase 1) | | | |
| MEIOB | meiosis specific with OB domains | −5.91689 | −2.56484 | 0.000871 |
| CADPS2 | Ca++-dependent secretion activator 2 | −5.91427 | −2.5642 | 2.11E−06 |
| LOC729041 | NA | −5.8972 | −2.56003 | 5.46E−05 |
| QPCT | glutaminyl-peptide cyclotransferase | −5.89426 | −2.55931 | 1.37E−28 |
| HOXA5 | homeobox A5 | −5.89393 | −2.55923 | 1.56E−22 |
| SOX18 | SRY (sex determining region Y)-box 18 | −5.88099 | −2.55606 | 4.33E−11 |
| GOLGA8S | golgin A8 family_member S | −5.87724 | −2.55514 | 0.008437 |
| EMR2 | adhesion G protein-coupled receptor E2 | −5.868 | −2.55287 | 0.001436 |
| GOLGA8M | golgin A8 family_member M | −5.86394 | −2.55187 | 0.001897 |
| LOXL3 | lysyl oxidase-like 3 | −5.85931 | −2.55073 | 1.05E−25 |
| CD70 | CD70 molecule | −5.8535 | −2.5493 | 0.000669 |
| CRHR2 | corticotropin releasing hormone receptor 2 | −5.85204 | −2.54894 | 0.004334 |
| TUSC1 | tumor suppressor candidate 1 | −5.83742 | −2.54533 | 4.43E−32 |
| OPCML | opioid binding protein/cell adhesion molecule- | −5.80964 | −2.53845 | 7.19E−05 |
| | like | | | |
| RASD1 | RAS_dexamethasone-induced 1 | −5.78987 | −2.53353 | 2.46E−15 |
| RASIP1 | Ras interacting protein 1 | −5.77736 | −2.53041 | 0.003568 |
| C8orf34 | chromosome 8 open reading frame 34 | −5.77396 | −2.52956 | 2.92E−19 |
| LINC00341 | long intergenic non-protein coding RNA 341 | −5.77043 | −2.52868 | 2.78E−24 |
| THPO | thrombopoietin | −5.72561 | −2.51743 | 1.25E−05 |
| KRT38 | keratin 38_type I | −5.71245 | −2.51411 | 0.008435 |
| LOC100506746 | uncharacterized LOC100506746 | −5.69261 | −2.50909 | 2.59E−10 |
| ACTR3C | ARP3 actin-related protein 3 homolog C | −5.68878 | −2.50812 | 0.000316 |
| | (yeast) | | | |
| GPR78 | G protein-coupled receptor 78 | −5.67866 | −2.50555 | 0.002572 |
| HAS2-AS1 | HAS2 antisense RNA 1 | −5.65148 | −2.49863 | 9.77E−20 |
| CACNA1G | calcium channel_voltage-dependent_T | −5.62588 | −2.49208 | 0.000806 |
| | type_alpha 1G subunit | | | |
| C8orf31 | chromosome 8 open reading frame 31 | −5.62472 | −2.49178 | 1.44E−19 |
| DNAJC6 | DnaJ (Hsp40) homolog_subfamily | −5.60949 | −2.48787 | 1.16E−20 |
| | C_member 6 | | | |
| PSTPIP1 | proline-serine-threonine phosphatase | −5.60114 | −2.48572 | 1.32E−09 |
| | interacting protein 1 | | | |
| WDR96 | cilia and flagella associated protein 43 | −5.58242 | −2.48089 | 1.59E−05 |
| DMKN | dermokine | −5.58029 | −2.48034 | 2.46E−06 |
| ASIC4 | acid sensing (proton gated) ion channel family | −5.57596 | −2.47922 | 8.95E−06 |
| | member 4 | | | |
| LOC100132352 | NA | −5.56955 | −2.47756 | 1.56E−18 |
| CCDC170 | coiled-coil domain containing 170 | −5.55397 | −2.47352 | 5.97E−15 |
| VEGFA | vascular endothelial growth factor A | −5.53138 | −2.46764 | 1.95E−13 |
| SLC6A9 | solute carrier family 6 (neurotransmitter | −5.53119 | −2.46759 | 4.84E−36 |
| | transporter_glycine)_member 9 | | | |
| EIF4EBP3 | eukaryotic translation initiation factor 4E | −5.51362 | −2.463 | 1.18E−06 |
| | binding protein 3 | | | |
| MMP2 | matrix metallopeptidase 2 | −5.50976 | −2.46199 | 6.16E−19 |
| SLC15A3 | solute carrier family | −5.50923 | −2.46185 | 2.06E−21 |
| | 15 (oligopeptide | | | |
| | transporter)_member 3 | | | |
| MIR3074 | microRNA 3074 | −5.49504 | −2.45813 | 0.000669 |
| LINC00707 | long intergenic non-protein coding RNA 707 | −5.48766 | −2.45619 | 0.004664 |
| SNHG5 | small nucleolar RNA host gene 5 | −5.47478 | −2.4528 | 6.87E−05 |
| IRAK3 | interleukin-1 receptor-associated kinase 3 | −5.45792 | −2.44835 | 7.59E−26 |
| AK4 | adenylate kinase 4 | −5.44824 | −2.44579 | 8.23E−19 |
| GALNT1 | polypeptide N-acetylgalactosaminyltransferase | −5.43526 | −2.44235 | 5.15E−11 |
| | 1 | | | |
| NR1D1 | nuclear receptor subfamily 1_group | −5.43255 | −2.44163 | 3.99E−26 |
| | D_member 1 | | | |
| SOCS2-AS1 | SOCS2 antisense RNA 1 | −5.42758 | −2.44031 | 3.48E−10 |
| CLMP | CXADR-like membrane protein | −5.42382 | −2.43931 | 9.55E−08 |
| LOC101929125 | uncharacterized LOC101929125 | −5.40416 | −2.43407 | 4.93E−05 |
| ZNF568 | zinc finger protein 568 | −5.39982 | −2.43291 | 4.37E−64 |
| PTER | phosphotriesterase related | −5.39925 | −2.43276 | 1.36E−11 |
| GOLGA6L4 | golgin A6 family-like 4 | −5.39256 | −2.43097 | 1.00E−05 |
| CASP1 | caspase 1_apoptosis-related cysteine peptidase | −5.39103 | −2.43056 | 3.33E−15 |
| LINC01152 | long intergenic non-protein coding RNA 1152 | −5.38774 | −2.42968 | 0.000239 |
| EFHD1 | EF-hand domain family_member D1 | −5.38662 | −2.42938 | 0.000127 |
| TMTC1 | transmembrane and tetratricopeptide repeat | −5.37588 | −2.4265 | 4.69E−09 |
| | containing 1 | | | |
| HOTAIR | HOX transcript antisense RNA | −5.34882 | −2.41922 | 2.75E−14 |
| PRKD1 | protein kinase D1 | −5.34804 | −2.41901 | 1.67E−75 |
| LOC102724316 | NA | −5.31861 | −2.41105 | 3.64E−148 |
| FAM69A | family with sequence similarity 69_member A | −5.31555 | −2.41022 | 1.23E−95 |
| ODF3L2 | outer dense fiber of sperm tails 3-like 2 | −5.30963 | −2.40861 | 1.50E−05 |
| LOC101928414 | uncharacterized LOC101928414 | −5.29808 | −2.40547 | 0.006814 |
| PLCL1 | phospholipase C-like 1 | −5.29555 | −2.40478 | 9.84E−08 |
| NCF2 | neutrophil cytosolic factor 2 | −5.27361 | −2.39879 | 2.58E−15 |
| LOC101241902 | chromosome 4 open reading frame 46 | −5.27046 | −2.39793 | 1.49E−06 |
| | pseudogene | | | |
| PRR15 | proline rich 15 | −5.26802 | −2.39726 | 1.01E−05 |
| SERPINE2 | serpin peptidase inhibitor_clade E | −5.26685 | −2.39694 | 5.29E−24 |
| | (nexin_plasminogen activator inhibitor | | | |
| | type 1)_member 2 | | | |
| CYP4V2 | cytochrome P450_family 4_subfamily | −5.25992 | −2.39504 | 1.24E−15 |
| | V_polypeptide 2 | | | |
| DENND2C | DENN/MADD domain containing 2C | −5.2427 | −2.39031 | 5.92E−07 |
| SBSN | suprabasin | −5.24263 | −2.39029 | 7.29E−08 |
| PDGFRA | platelet-derived growth factor receptor_alpha | −5.20862 | −2.3809 | 2.21E−168 |
| | polypeptide | | | |
| MYOM1 | myomesin 1 | −5.19942 | −2.37835 | 4.14E−07 |
| COL6A3 | collagen_type VI_alpha 3 | −5.19621 | −2.37746 | 6.71E−55 |
| MIR6775 | microRNA 6775 | −5.18477 | −2.37428 | 0.000735 |
| LINC00921 | long intergenic non-protein coding RNA 921 | −5.15046 | −2.3647 | 1.26E−14 |
| LINC01352 | long intergenic non-protein coding RNA 1352 | −5.13232 | −2.35961 | 7.17E−06 |
| NXPH3 | neurexophilin 3 | −5.12915 | −2.35872 | 6.51E−10 |
| LOC100507557 | uncharacterized LOC100507557 | −5.12826 | −2.35847 | 7.49E−14 |
| DHRS4L1 | dehydrogenase/reductase (SDR family) | −5.11906 | −2.35588 | 0.004779 |
| | member 4 like 1 | | | |
| TXNRD2 | thioredoxin reductase 2 | −5.07937 | −2.34465 | 1.58E−31 |
| PCDHA3 | protocadherin alpha 3 | −5.07779 | −2.3442 | 0.000773 |
| ALDH1A3 | aldehyde dehydrogenase 1 family_member A3 | −5.0685 | −2.34156 | 1.92E−06 |
| PPFIA2 | protein tyrosine phosphatase_receptor type_f | −5.06538 | −2.34067 | 4.69E−05 |
| | polypeptide (PTPRF)_interacting protein | | | |
| | (liprin)_alpha 2 | | | |
| TLE3 | transducin-like enhancer of split 3 | −5.06183 | −2.33966 | 9.92E−78 |
| CLDN23 | claudin 23 | −5.05927 | −2.33893 | 0.000316 |
| STEAP1 | six transmembrane epithelial antigen of the | −5.04544 | −2.33498 | 3.27E−44 |
| | prostate 1 | | | |
| ADAMTS9- | ADAMTS9 antisense RNA 2 | −5.04177 | −2.33393 | 4.23E−06 |
| AS2 | | | | |
| ANK2 | ankyrin 2_neuronal | −5.03583 | −2.33223 | 6.76E−28 |
| FCRLA | Fc receptor-like A | −5.02004 | −2.3277 | 8.25E−08 |
| UNC5C | unc-5 netrin receptor C | −5.01017 | −2.32486 | 1.67E−05 |
| ATOH8 | atonal bHLH transcription factor 8 | −5.0049 | −2.32334 | 6.40E−56 |

TABLE 5
Genes more highly expressed in HMCs compared to UCB-MSCs

| | | | Log | |
|---|---|---|---|---|
| | | Fold | Fold | |
| Name | Description | Change | Change | p-Value |
| LRRN1 | leucine rich repeat neuronal 1 | 3423.37 | 11.7412 | 7.84E−104 |
| NKX2-5 | NK2 homeobox 5 | 1581.96 | 10.6275 | 6.39E−97 |
| IGFBP2 | insulin-like growth factor binding | 1184.37 | 10.2099 | 2.47E−103 |
| | protein 2_ 36 kDa | | | |
| DCC | DCC netrin 1 receptor | 891.771 | 9.80053 | 1.60E−76 |
| NETO1 | neuropilin (NRP) and tolloid | 852.709 | 9.73591 | 3.52E−68 |
| | (TLL)-like 1 | | | |
| IGSF1 | immunoglobulin superfamily_ member 1 | 611.14 | 9.25536 | 7.03E−52 |
| LOC440416 | NA | 540.215 | 9.07739 | 3.70E−139 |
| FLJ16779 | uncharacterized LOC100192386 | 430.748 | 8.7507 | 3.08E−52 |
| NKAIN4 | Na+/K+ transporting ATPase interacting | 369.492 | 8.5294 | 9.57E−49 |
| | 4 | | | |
| OCA2 | oculocutaneous albinism II | 359.59 | 8.49021 | 2.24E−89 |
| NLGN4X | neuroligin 4_ X-linked | 350.92 | 8.455 | 1.14E−41 |
| RSPO4 | R-spondin 4 | 313.369 | 8.29172 | 2.78E−76 |
| LIN28B | lin-28 homolog B (C. elegans) | 307.263 | 8.26333 | 2.37E−52 |
| KCTD8 | potassium channel tetramerization | 297.083 | 8.21472 | 1.55E−48 |
| | domain containing 8 | | | |
| IRX2 | iroquois homeobox 2 | 237.351 | 7.89088 | 1.80E−48 |
| PLAC8 | placenta-specific 8 | 207.368 | 7.69605 | 2.30E−74 |
| CLSTN2 | calsyntenin 2 | 201.99 | 7.65814 | 4.29E−113 |
| CACNG4 | calcium channel_ voltage-dependent_ | 174.326 | 7.44564 | 5.03E−71 |
| | gamma subunit 4 | | | |
| PHOX2A | paired-like homeobox 2a | 169.602 | 7.40601 | 2.70E−36 |
| ITGA8 | integrin_ alpha 8 | 169.257 | 7.40307 | 4.76E−40 |
| CHRDL1 | chordin-like 1 | 159.108 | 7.31386 | 2.02E−44 |
| UNC5C | unc-5 netrin receptor C | 150.173 | 7.23048 | 7.96E−46 |
| NLRP2 | NLR family_ pyrin domain containing 2 | 147.386 | 7.20346 | 3.25E−30 |
| PRAC1 | prostate cancer susceptibility candidate 1 | 136.827 | 7.09621 | 3.19E−25 |
| PCDHB2 | protocadherin beta 2 | 130.227 | 7.02488 | 3.62E−25 |
| TRPC5 | transient receptor potential cation | 127.06 | 6.98937 | 3.76E−30 |
| | channel_ subfamily C_ member 5 | | | |
| PPARGC1A | peroxisome proliferator-activated | 124.471 | 6.95967 | 4.68E−32 |
| | receptor gamma_ coactivator 1 alpha | | | |
| NRK | Nik related kinase | 122.669 | 6.93863 | 5.98E−41 |
| ABCB1 | ATP-binding cassette_ sub-family B | 122.107 | 6.932 | 2.34E−39 |
| | (MDR/TAP)_ member 1 | | | |
| PALM | paralemmin | 112.71 | 6.81647 | 2.44E−94 |
| LRRTM1 | leucine rich repeat transmembrane | 112.66 | 6.81583 | 1.31E−68 |
| | neuronal 1 | | | |
| LOC642366 | uncharacterized LOC642366 | 109.152 | 6.77019 | 7.96E−38 |
| KCNK3 | potassium channel_ two pore domain | 107.071 | 6.74242 | 5.85E−41 |
| | subfamily K_ member 3 | | | |
| SIX1 | SIX homeobox 1 | 105.882 | 6.72631 | 1.43E−71 |
| SLC44A5 | solute carrier family 44_ member 5 | 105.792 | 6.72509 | 3.28E−75 |
| OVCH2 | ovochymase 2 (gene/pseudogene) | 105.433 | 6.72018 | 2.03E−45 |
| PRDM16 | PR domain containing 16 | 104.665 | 6.70963 | 2.54E−63 |
| MGAM | maltase-glucoamylase | 100.991 | 6.65809 | 1.29E−46 |
| GCNT2 | glucosaminyl (N-acetyl) transferase 2_ | 99.6577 | 6.63891 | 5.38E−48 |
| | I-branching enzyme (I blood group) | | | |
| TNRC6C- | TNRC6C antisense RNA 1 | 99.3178 | 6.63398 | 1.80E−33 |
| AS1 | | | | |
| ANO1 | anoctamin 1_calcium activated chloride | 97.8208 | 6.61207 | 3.23E−44 |
| | channel | | | |
| GATA3- | GATA3 antisense RNA 1 | 97.6731 | 6.60989 | 1.41E−29 |
| AS1 | | | | |
| EBF3 | early B-cell factor 3 | 95.5471 | 6.57814 | 5.38E−33 |
| SPINK5 | serine peptidase inhibitor_ Kazal type 5 | 91.5539 | 6.51655 | 6.26E−18 |
| FXYD6 | FXYD domain containing ion transport | 86.0701 | 6.42744 | 1.97E−22 |
| | regulator 6 | | | |
| SLITRK1 | SLIT and NTRK-like family_ member 1 | 84.3333 | 6.39803 | 2.73E−28 |
| DPPA4 | developmental pluripotency associated 4 | 83.2928 | 6.38012 | 2.54E−16 |
| NKX2-6 | NK2 homeobox 6 | 77.2391 | 6.27126 | 9.65E−21 |
| SYT13 | synaptotagmin XIII | 75.1088 | 6.23091 | 2.59E−17 |
| LGR5 | leucine-rich repeat containing G protein- | 74.3515 | 6.21629 | 1.30E−16 |
| | coupled receptor 5 | | | |
| LHX2 | LIM homeobox 2 | 73.4001 | 6.19771 | 1.19E−30 |
| CYTIP | cytohesin 1 interacting protein | 72.9805 | 6.18944 | 4.02E−19 |
| BMP2 | bone morphogenetic protein 2 | 72.9274 | 6.18839 | 2.01E−33 |
| CST1 | cystatin SN | 71.2699 | 6.15522 | 1.75E−18 |
| AFF3 | AF4/FMR2 family_ member 3 | 70.7339 | 6.14433 | 4.25E−45 |
| TMEM132B | transmembrane protein 132B | 66.6337 | 6.05818 | 1.53E−33 |
| ADAMTS18 | ADAM metallopeptidase with | 65.9833 | 6.04403 | 1.11E−27 |
| | thrombospondin type 1 motif_ 18 | | | |
| C8orf4 | chromosome 8 open reading frame 4 | 65.9737 | 6.04382 | 5.03E−27 |
| CDH10 | cadherin 10_ type 2 (T2-cadherin) | 64.672 | 6.01507 | 2.27E−20 |
| PDE1C | phosphodiesterase 1C_ calmodulin- | 64.3452 | 6.00776 | 1.34E−91 |
| | dependent 70 kDa | | | |
| PLCXD3 | phosphatidylinositol-specific | 63.3025 | 5.98419 | 3.70E−17 |
| | phospholipase C_ X domain containing | | | |
| | 3 | | | |
| SH2D3C | SH2 domain containing 3C | 63.288 | 5.98386 | 5.85E−27 |
| P2RY14 | purinergic receptor P2Y_ G-protein | 62.0216 | 5.9547 | 7.33E−17 |
| | coupled_ 14 | | | |
| VIT | vitrin | 61.9138 | 5.95219 | 1.55E−29 |
| TLR4 | toll-like receptor 4 | 61.5135 | 5.94283 | 1.08E−28 |
| PKIB | protein kinase (cAMP-dependent_ | 61.1347 | 5.93392 | 5.81E−30 |
| | catalytic) inhibitor beta | | | |
| C5orf38 | chromosome 5 open reading frame 38 | 60.5666 | 5.92045 | 3.12E−23 |
| KCNA1 | potassium channel_ voltage gated shaker | 60.5552 | 5.92018 | 9.27E−20 |
| | related subfamily A_ member 1 | | | |
| CDH3 | cadherin 3_ type 1_ P-cadherin | 58.9647 | 5.88178 | 4.90E−23 |
| | (placental) | | | |
| CD24 | CD24 molecule | 58.1703 | 5.86221 | 1.21E−27 |
| PCDHA12 | protocadherin alpha 12 | 57.8257 | 5.85364 | 3.85E−17 |
| LINC00491 | long intergenic non-protein coding RNA | 56.8741 | 5.8297 | 9.86E−16 |
| | 491 | | | |
| COL22A1 | collagen_ type XXII_ alpha 1 | 56.2589 | 5.81401 | 6.75E−16 |
| LHX1 | LIM homeobox 1 | 55.9249 | 5.80542 | 4.65E−21 |
| CYP27C1 | cytochrome P450_ family 27_ subfamily | 55.3101 | 5.78947 | 1.14E−14 |
| | C_ polypeptide 1 | | | |
| CRHBP | corticotropin releasing hormone binding | 53.735 | 5.74779 | 3.46E−16 |
| | protein | | | |
| RERG | RAS-like_ estrogen-regulated_ growth | 53.574 | 5.74346 | 2.81E−21 |
| | inhibitor | | | |
| LOC644919 | uncharacterized LOC644919 | 52.9644 | 5.72695 | 1.01E−28 |
| FRMPD3 | FERM and PDZ domain containing 3 | 52.182 | 5.70548 | 6.36E−29 |
| GABRG3 | gamma-aminobutyric acid (GABA) A | 51.9283 | 5.69845 | 8.06E−15 |
| | receptor_ gamma 3 | | | |
| CHST15 | carbohydrate (N-acetylgalactosamine 4- | 51.5446 | 5.68775 | 9.64E−69 |
| | sulfate 6-O) sulfotransferase 15 | | | |
| C14orf39 | chromosome 14 open reading frame 39 | 51.4707 | 5.68568 | 1.34E−32 |
| SLC5A12 | solute carrier family 5 | 50.7533 | 5.66543 | 6.50E−28 |
| | (sodium/monocarboxylate | | | |
| | cotransporter)_ member 12 | | | |
| ST8SIA2 | ST8 alpha-N-acetyl-neuraminide alpha- | 50.7101 | 5.6642 | 2.57E−15 |
| | 2_8-sialyltransferase 2 | | | |
| SFRP1 | secreted frizzled-related protein 1 | 48.7693 | 5.6079 | 1.72E−51 |
| SLCO6A1 | solute carrier organic anion transporter | 48.3763 | 5.59623 | 1.56E−13 |
| | family_ member 6A1 | | | |
| KIAA0040 | KIAA0040 | 48.2565 | 5.59265 | 3.37E−16 |
| FBP2 | fructose-1_6-bisphosphatase 2 | 48.0722 | 5.58713 | 1.41E−20 |
| ANKRD1 | ankyrin repeat domain 1 (cardiac | 47.3197 | 5.56437 | 3.90E−29 |
| | muscle) | | | |
| TMEM40 | transmembrane protein 40 | 47.1841 | 5.56023 | 1.11E−27 |
| SLC1A7 | solute carrier family 1 (glutamate | 46.3199 | 5.53356 | 6.47E−25 |
| | transporter)_ member 7 | | | |
| PODN | podocan | 46.2856 | 5.53249 | 5.78E−87 |
| SFMBT2 | Scm-like with four mbt domains 2 | 46.1078 | 5.52694 | 2.44E−28 |
| NKX3-2 | NK3 homeobox 2 | 45.6483 | 5.51249 | 4.78E−22 |
| SHC2 | SHC (Src homology 2 domain | 45.3695 | 5.50365 | 6.32E−54 |
| | containing) transforming protein 2 | | | |
| SLCO2A1 | solute carrier organic anion transporter | 44.7573 | 5.48405 | 3.12E−23 |
| | family_ member 2A1 | | | |
| MYCT1 | myc target 1 | 44.739 | 5.48346 | 1.75E−22 |
| FIRRE | firre intergenic repeating RNA element | 43.2066 | 5.43318 | 2.76E−15 |
| TNNI1 | troponin I type 1 (skeletal_ slow) | 42.8853 | 5.42241 | 2.00E−23 |
| BCL11B | B-cell CLL/lymphoma 11B (zinc finger | 42.833 | 5.42065 | 2.47E−14 |
| | protein) | | | |
| ISL1 | ISL LIM homeobox 1 | 42.4758 | 5.40857 | 2.02E−12 |
| CLEC1A | C-type lectin domain family 1_ member | 42.2799 | 5.4019 | 5.81E−13 |
| | A | | | |
| TSPAN11 | tetraspanin 11 | 41.6233 | 5.37932 | 1.52E−37 |
| KRTAP1-1 | keratin associated protein 1-1 | 41.5841 | 5.37796 | 1.11E−23 |
| HS6ST2 | heparan sulfate 6-O-sulfotransferase 2 | 41.4563 | 5.37352 | 1.08E−21 |
| PCDHA4 | protocadherin alpha 4 | 40.8944 | 5.35383 | 5.99E−17 |
| WSCD1 | WSC domain containing 1 | 40.5031 | 5.33996 | 6.93E−22 |
| MED15P9 | mediator complex subunit 15 | 39.4893 | 5.30339 | 3.88E−11 |
| | pseudogene 9 | | | |
| PLP1 | proteolipid protein 1 | 39.4054 | 5.30032 | 4.24E−21 |
| NIPAL4 | NIPA-like domain containing 4 | 39.3494 | 5.29827 | 1.36E−59 |
| FAR2P1 | fatty acyl CoA reductase 2 pseudogene 1 | 39.2938 | 5.29623 | 4.11E−11 |
| LINC01096 | long intergenic non-protein coding RNA | 38.8828 | 5.28106 | 7.17E−14 |
| | 1096 | | | |
| MMP9 | matrix metallopeptidase 9 | 38.4633 | 5.26541 | 1.22E−50 |
| VAV3 | vav 3 guanine nucleotide exchange | 38.3209 | 5.26006 | 8.17E−19 |
| | factor | | | |
| C7 | complement component 7 | 38.2986 | 5.25922 | 2.82E−18 |
| TBX15 | T-box 15 | 37.8573 | 5.2425 | 4.62E−19 |
| CASC9 | cancer susceptibility candidate 9 (non- | 37.6386 | 5.23414 | 3.36E−15 |
| | protein coding) | | | |
| DIO2 | deiodinase_ iodothyronine_ type II | 36.187 | 5.1774 | 2.13E−67 |
| LIPG | lipase_ endothelial | 36.1381 | 5.17545 | 3.51E−58 |
| GCNT4 | glucosaminyl (N-acetyl) transferase 4_ | 36.0191 | 5.17069 | 1.91E−24 |
| | core 2 | | | |
| MYH14 | myosin_ heavy chain 14_ non-muscle | 35.5979 | 5.15372 | 6.48E−15 |
| A2M | alpha-2-macroglobulin | 35.1412 | 5.13509 | 4.63E−16 |
| LINC01021 | long intergenic non-protein coding RNA | 34.8127 | 5.12154 | 2.04E−23 |
| | 1021 | | | |
| FAM65B | family with sequence similarity 65_ | 34.4156 | 5.10499 | 7.07E−43 |
| | member B | | | |
| GNA14 | guanine nucleotide binding protein (G | 34.2393 | 5.09758 | 2.18E−36 |
| | protein)_ alpha 14 | | | |
| FAT3 | FAT atypical cadherin 3 | 33.7873 | 5.07841 | 2.67E−22 |
| LINC00982 | long intergenic non-protein coding RNA | 33.7057 | 5.07492 | 1.23E−12 |
| | 982 | | | |
| TCEAL2 | transcription elongation factor A (SII)- | 33.0111 | 5.04488 | 2.67E−17 |
| | like 2 | | | |
| ZCCHC16 | zinc finger_ CCHC domain containing | 32.9462 | 5.04204 | 6.15E−12 |
| | 16 | | | |
| GPR112 | adhesion G protein-coupled receptor G4 | 32.5887 | 5.0263 | 2.27E−11 |
| PCDHB4 | protocadherin beta 4 | 31.9594 | 4.99817 | 3.18E−18 |
| CACNA1H | calcium channel_ voltage-dependent_ T | 31.9406 | 4.99732 | 9.26E−41 |
| | type_alpha 1H subunit | | | |
| SCARF1 | scavenger receptor class F_ member 1 | 31.5437 | 4.97928 | 3.39E−90 |
| SHISA3 | shisa family member 3 | 31.1437 | 4.96087 | 5.28E−15 |
| KCNF1 | potassium channel_ voltage gated | 30.8845 | 4.94881 | 1.67E−15 |
| | modifier subfamily F_ member 1 | | | |
| B3GAT1 | beta-1_3-glucuronyltransferase 1 | 30.6098 | 4.93592 | 3.57E−20 |
| EXOC3L2 | exocyst complex component 3-like 2 | 30.5731 | 4.93419 | 9.95E−36 |
| TRIM55 | tripartite motif containing 55 | 30.4766 | 4.92963 | 5.00E−118 |
| PLXDC1 | plexin domain containing 1 | 30.4333 | 4.92758 | 1.81E−26 |
| TBX1 | T-box 1 | 30.343 | 4.92329 | 8.72E−30 |
| SMOC1 | SPARC related modular calcium binding | 30.1614 | 4.91463 | 2.44E−17 |
| | 1 | | | |
| EFHD1 | EF-hand domain family_ member D1 | 29.6463 | 4.88978 | 4.18E−27 |
| CD93 | CD93 molecule | 29.3736 | 4.87645 | 2.59E−14 |
| KISS1 | KiSS-1 metastasis-suppressor | 28.5813 | 4.837 | 4.19E−12 |
| OR10A3 | olfactory receptor_ family 10_ | 28.1094 | 4.81298 | 4.22E−10 |
| | subfamily A_ member 3 | | | |
| LRRC4C | leucine rich repeat containing 4C | 27.9941 | 4.80705 | 7.72E−18 |
| BEX1 | brain expressed_ X-linked 1 | 26.7397 | 4.74091 | 9.64E−24 |
| TNNT2 | troponin T type 2 (cardiac) | 26.4701 | 4.72629 | 2.05E−21 |
| RBM20 | RNA binding motif protein 20 | 25.9298 | 4.69654 | 2.80E−25 |
| TMC6 | transmembrane channel-like 6 | 25.8548 | 4.69236 | 1.86E−27 |
| TMEM200C | transmembrane protein 200C | 25.7952 | 4.68903 | 2.92E−12 |
| LINGO1 | leucine rich repeat and Ig domain | 25.5052 | 4.67272 | 1.30E−100 |
| | containing 1 | | | |
| CNNM1 | cyclin and CBS domain divalent metal | 25.5017 | 4.67252 | 1.63E−28 |
| | cation transport mediator 1 | | | |
| PCDHA11 | protocadherin alpha 11 | 24.9492 | 4.64092 | 7.22E−10 |
| FAM19A5 | family with sequence similarity 19 | 24.8579 | 4.63563 | 4.90E−20 |
| | (chemokine (C-C motif)-like)_ member | | | |
| | A5 | | | |
| DACT2 | dishevelled-binding antagonist of beta- | 24.8207 | 4.63347 | 2.24E−09 |
| | catenin 2 | | | |
| BRINP1 | bone morphogenetic protein/retinoic | 24.5667 | 4.61863 | 4.07E−10 |
| | acid inducible neural-specific 1 | | | |
| CDH5 | cadherin 5_ type 2 (vascular | 24.4423 | 4.61131 | 1.18E−09 |
| | endothelium) | | | |
| ZMAT1 | zinc finger_ matrin-type 1 | 24.4046 | 4.60908 | 6.07E−14 |
| SHISA2 | shisa family member 2 | 24.293 | 4.60247 | 5.61E−17 |
| NUTM2F | NUT family member 2F | 24.1311 | 4.59282 | 4.34E−10 |
| NNAT | neuronatin | 23.7076 | 4.56728 | 1.98E−30 |
| LGI1 | leucine-rich_ glioma inactivated 1 | 23.391 | 4.54788 | 5.91E−13 |
| MAP2 | microtubule-associated protein 2 | 23.3814 | 4.54729 | 6.52E−74 |
| KC6 | keratoconus gene 6 | 23.3338 | 4.54435 | 1.16E−14 |
| LPPR3 | lipid phosphate phosphatase-related | 23.2252 | 4.53762 | 8.02E−25 |
| | protein type 3 | | | |
| PARVG | parvin_gamma | 22.8769 | 4.51582 | 6.30E−11 |
| EXTL 1 | exostosin-like glycosyltransferase 1 | 22.7777 | 4.50955 | 3.33E−26 |
| BAI3 | adhesion G protein-coupled receptor B3 | 22.6455 | 4.50115 | 1.26E−13 |
| ITIH3 | inter-alpha-trypsin inhibitor heavy chain | 22.6251 | 4.49985 | 1.71E−35 |
| | 3 | | | |
| LOC339166 | uncharacterized LOC339166 | 22.4073 | 4.4859 | 5.90E−12 |
| GJA5 | gap junction protein_alpha 5_40 kDa | 22.3805 | 4.48417 | 2.27E−09 |
| TTR | transthyretin | 22.3757 | 4.48386 | 4.57E−10 |
| LOC440910 | uncharacterized LOC440910 | 22.3751 | 4.48382 | 1.60E−08 |
| NOVA1 | neuro-oncological ventral antigen 1 | 22.1041 | 4.46624 | 4.48E−09 |
| PCDH17 | protocadherin 17 | 22.0883 | 4.46521 | 1.44E−12 |
| ERP27 | endoplasmic reticulum protein 27 | 21.8318 | 4.44836 | 2.90E−15 |
| SLC37A1 | solute carrier family 37 (glucose-6- | 21.6711 | 4.4377 | 1.39E−33 |
| | phosphate transporter)_ member 1 | | | |
| MMP23B | matrix metallopeptidase 23B | 21.3419 | 4.41562 | 7.23E−22 |
| SHOX2 | short stature homeobox 2 | 21.0896 | 4.39846 | 6.22E−14 |
| PDE9A | phosphodiesterase 9A | 20.9168 | 4.38659 | 8.62E−14 |
| GPR37 | G protein-coupled receptor 37 | 20.6854 | 4.37054 | 2.43E−16 |
| | (endothelin receptor type B-like) | | | |
| KRTAP4- | keratin associated protein 4-12 | 20.5994 | 4.36453 | 1.38E−09 |
| 12 | | | | |
| ABCB4 | ATP-binding cassette_ sub-family B | 20.596 | 4.36429 | 7.71E−14 |
| | (MDR/TAP)_ member 4 | | | |
| LOC283299 | uncharacterized LOC283299 | 20.5908 | 4.36393 | 7.84E−11 |
| CXXC4 | CXXC finger protein 4 | 20.5032 | 4.35778 | 4.39E−12 |
| LOC101928340 | NA | 20.221 | 4.33778 | 3.54E−08 |
| GRIN2A | glutamate receptor_ ionotropic_N- | 20.0934 | 4.32865 | 7.56E−09 |
| | methyl D-aspartate 2A | | | |
| FCGBP | Fc fragment of IgG binding protein | 20.0563 | 4.32598 | 5.12E−16 |
| LOC102467080 | uncharacterized LOC102467080 | 20.0368 | 4.32458 | 7.29E−32 |
| KIT | v-kit Hardy-Zuckerman 4 feline sarcoma | 19.7848 | 4.30632 | 1.51E−14 |
| | viral oncogene homolog | | | |
| ANO5 | anoctamin 5 | 19.7803 | 4.30599 | 3.21E−08 |
| SALL1 | spalt-like transcription factor 1 | 19.6367 | 4.29548 | 1.26E−19 |
| EMCN | endomucin | 19.4577 | 4.28227 | 6.72E−10 |
| PLXNA4 | plexin A4 | 19.3804 | 4.27653 | 7.04E−24 |
| NR0B1 | nuclear receptor subfamily 0_ group B_ | 19.2784 | 4.26891 | 1.26E−08 |
| | member 1 | | | |
| MDGA2 | MAM domain containing | 19.1619 | 4.26017 | 2.68E−24 |
| | glycosylphosphatidylinositol anchor 2 | | | |
| FAM49A | family with sequence similarity 49_ | 19.0858 | 4.25443 | 4.32E−58 |
| | member A | | | |
| KSR2 | kinase suppressor of ras 2 | 18.8714 | 4.23813 | 4.77E−09 |
| AIF1L | allograft inflammatory factor 1-like | 18.8237 | 4.23448 | 2.05E−21 |
| DAAM2 | dishevelled associated activator of | 18.6607 | 4.22193 | 2.05E−45 |
| | morphogenesis 2 | | | |
| IGDCC3 | immunoglobulin superfamily_ DCC | 18.5723 | 4.21508 | 1.08E−10 |
| | subclass_ member 3 | | | |
| GDF7 | growth differentiation factor 7 | 18.421 | 4.20328 | 3.48E−08 |
| MGAT4C | MGAT4 family_ member C | 18.3217 | 4.19548 | 1.72E−08 |
| LDB3 | LIM domain binding 3 | 18.287 | 4.19275 | 1.11E−40 |
| DENND2A | DENN/MADD domain containing 2A | 18.2434 | 4.1893 | 3.62E−27 |
| OR5E1P | olfactory receptor_ family 5_ subfamily | 18.1705 | 4.18353 | 4.88E−09 |
| | E_ member 1 pseudogene | | | |
| SYTL5 | synaptotagmin-like 5 | 18.1419 | 4.18125 | 8.91E−19 |
| TNFSF18 | tumor necrosis factor (ligand) | 18.1251 | 4.17992 | 1.85E−11 |
| | superfamily_ member 18 | | | |
| RELN | reelin | 17.9466 | 4.16564 | 5.93E−14 |
| IRX1 | iroquois homeobox 1 | 17.9075 | 4.16249 | 1.27E−07 |
| LARGE | like-glycosyltransferase | 17.8339 | 4.15655 | 1.82E−39 |
| FAM69B | family with sequence similarity 69_ | 17.7295 | 4.14808 | 1.72E−47 |
| | member B | | | |
| SULT1C4 | sulfotransferase family_ cytosolic_ 1C_ | 17.4646 | 4.12636 | 2.56E−07 |
| | member 4 | | | |
| EMID1 | EMI domain containing 1 | 17.2871 | 4.11162 | 1.05E−20 |
| MGAT3 | mannosyl (beta-1_4-)-glycoprotein beta- | 17.2126 | 4.10539 | 9.53E−45 |
| | 1_4-N-acetylglucosaminyltransferase | | | |
| ILDR2 | immunoglobulin-like domain containing | 16.8161 | 4.07177 | 4.49E−08 |
| | receptor 2 | | | |
| PLCB2 | phospholipase C_ beta 2 | 16.794 | 4.06987 | 9.31E−33 |
| EPCAM | epithelial cell adhesion molecule | 16.6721 | 4.05936 | 2.18E−19 |
| EPB41L3 | erythrocyte membrane protein band 4.1- | 16.6397 | 4.05656 | 6.17E−13 |
| | like 3 | | | |
| LICAM | L1 cell adhesion molecule | 16.6355 | 4.05619 | 8.47E−30 |
| BEX5 | brain expressed_ X-linked 5 | 16.586 | 4.05189 | 1.99E−07 |
| GFRA2 | GDNF family receptor alpha 2 | 16.5833 | 4.05166 | 1.29E−08 |
| DLX5 | distal-less homeobox 5 | 16.5784 | 4.05123 | 1.62E−07 |
| DLX1 | distal-less homeobox 1 | 16.5368 | 4.04761 | 7.18E−31 |
| GRIA1 | glutamate receptor_ ionotropic_ AMPA | 16.5334 | 4.04731 | 7.77E−11 |
| | 1 | | | |
| GRAP | GRB2-related adaptor protein | 16.4563 | 4.04057 | 2.92E−19 |
| BBOX1 | butyrobetaine (gamma)_ 2-oxoglutarate | 16.3933 | 4.03503 | 7.12E−09 |
| | dioxygenase (gamma-butyrobetaine | | | |
| | hydroxylase) 1 | | | |
| ADAMTS20 | ADAM metallopeptidase with | 16.3915 | 4.03488 | 7.59E−12 |
| | thrombospondin type 1 motif_ 20 | | | |
| CXCL12 | chemokine (C-X-C motif) ligand 12 | 16.3253 | 4.02904 | 4.11E−138 |
| UNC13A | unc-13 homolog A (C. elegans) | 16.2647 | 4.02367 | 1.32E−14 |
| RGS1 | regulator of G-protein signaling 1 | 16.2524 | 4.02258 | 5.09E−07 |
| DLX6 | distal-less homeobox 6 | 16.1897 | 4.017 | 5.02E−07 |
| GRB14 | growth factor receptor-bound protein 14 | 16.1678 | 4.01505 | 4.61E−15 |
| HUNK | hormonally up-regulated Neu-associated | 15.9866 | 3.99879 | 6.11E−14 |
| | kinase | | | |
| HEPH | hephaestin | 15.8794 | 3.98908 | 4.82E−07 |
| SLC6A16 | solute carrier family 6_ member 16 | 15.8359 | 3.98513 | 1.45E−22 |
| RGMA | repulsive guidance molecule family | 15.6927 | 3.97202 | 8.19E−18 |
| | member a | | | |
| GPR87 | G protein-coupled receptor 87 | 15.6778 | 3.97065 | 8.41E−26 |
| PADI2 | peptidyl arginine deiminase_ type II | 15.645 | 3.96763 | 4.81E−15 |
| PTPN6 | protein tyrosine phosphatase_ non- | 15.6183 | 3.96517 | 2.28E−20 |
| | receptor type 6 | | | |
| SUCNR1 | succinate receptor 1 | 15.5191 | 3.95597 | 6.46E−07 |
| PALMD | palmdelphin | 15.5141 | 3.95551 | 1.40E−49 |
| MERTK | MER proto-oncogene_ tyrosine kinase | 15.509 | 3.95503 | 5.72E−14 |
| KCNC3 | potassium channel_ voltage gated Shaw | 15.4779 | 3.95214 | 7.69E−11 |
| | related subfamily C_ member 3 | | | |
| PCDHB3 | protocadherin beta 3 | 15.4368 | 3.9483 | 6.91E−09 |
| CILP2 | cartilage intermediate layer protein 2 | 15.2364 | 3.92945 | 1.66E−32 |
| MAF | v-maf avian musculoaponeurotic | 15.2303 | 3.92887 | 8.06E−17 |
| | fibrosarcoma oncogene homolog | | | |
| NTRK2 | neurotrophic tyrosine kinase_ receptor_ | 15.1355 | 3.91986 | 1.08E−07 |
| | type 2 | | | |
| SEMA3E | sema domain_immunoglobulin domain | 15.0925 | 3.91576 | 6.08E−10 |
| | (Ig)_ short basic domain_ secreted_ | | | |
| | (semaphorin) 3E | | | |
| C21orf90 | TSPEAR antisense RNA 2 | 15.0718 | 3.91378 | 1.82E−12 |
| PCDHB9 | protocadherin beta 9 | 15.0096 | 3.90781 | 6.79E−14 |
| SIX2 | SIX homeobox 2 | 14.933 | 3.90043 | 1.73E−07 |
| CALY | calcyon neuron-specific vesicular | 14.8895 | 3.89622 | 9.05E−19 |
| | protein | | | |
| PCAT1 | prostate cancer associated transcript 1 | 14.6893 | 3.87669 | 4.25E−08 |
| | (non-protein coding) | | | |
| GPRC5C | G protein-coupled receptor_ class C_ | 14.6602 | 3.87383 | 3.58E−07 |
| | group 5_member C | | | |
| NRN1 | neuritin 1 | 14.6458 | 3.87242 | 1.36E−11 |
| RIMS1 | regulating synaptic membrane | 14.6198 | 3.86985 | 2.43E−22 |
| | exocytosis 1 | | | |
| LINC01012 | long intergenic non-protein coding RNA | 14.5829 | 3.86621 | 6.92E−09 |
| | 1012 | | | |
| SH3GL2 | SH3-domain GRB2-like 2 | 14.5492 | 3.86287 | 5.63E−09 |
| SYT3 | synaptotagmin III | 14.5396 | 3.86192 | 3.33E−12 |
| IL1RAPL1 | interleukin 1 receptor accessory protein- | 14.538 | 3.86176 | 4.34E−13 |
| | like 1 | | | |
| PART1 | prostate androgen-regulated transcript 1 | 14.5131 | 3.85928 | 9.40E−12 |
| | (non-protein coding) | | | |
| PCDHB10 | protocadherin beta 10 | 14.4383 | 3.85183 | 4.97E−17 |
| SRSF12 | serine/arginine-rich splicing factor 12 | 14.4059 | 3.84859 | 4.43E−16 |
| TRH | thyrotropin-releasing hormone | 14.405 | 3.8485 | 1.44E−06 |
| EPHB1 | EPH receptor B1 | 14.3437 | 3.84235 | 3.08E−13 |
| CD70 | CD70 molecule | 14.1423 | 3.82194 | 1.66E−06 |
| SPP1 | secreted phosphoprotein 1 | 14.1278 | 3.82046 | 7.35E−19 |
| DOC2GP | double C2-like domains_ gamma_ | 14.0595 | 3.81347 | 4.24E−10 |
| | pseudogene | | | |
| TSPEAR- | TSPEAR antisense RNA 1 | 14.0479 | 3.81228 | 4.84E−31 |
| AS1 | | | | |
| THBD | thrombomodulin | 14.0129 | 3.80868 | 1.12E−10 |
| RGS5 | regulator of G-protein signaling 5 | 14.0103 | 3.80842 | 1.90E−40 |
| CYP26B1 | cytochrome P450_ family 26_ subfamily | 13.9621 | 3.80344 | 3.95E−15 |
| | B_ polypeptide 1 | | | |
| LINC01139 | long intergenic non-protein coding RNA | 13.882 | 3.79514 | 1.74E−20 |
| | 1139 | | | |
| NAPIL2 | nucleosome assembly protein 1-like 2 | 13.8587 | 3.79272 | 2.85E−10 |
| MTUS1 | microtubule associated tumor suppressor | 13.7747 | 3.78395 | 3.49E−09 |
| | 1 | | | |
| DSP | desmoplakin | 13.7042 | 3.77655 | 2.30E−31 |
| AR | androgen receptor | 13.6558 | 3.77144 | 1.35E−28 |
| COL4A3 | collagen_ type IV_ alpha 3 | 13.5989 | 3.76542 | 1.62E−19 |
| | (Goodpasture antigen) | | | |
| PTH1R | parathyroid hormone 1 receptor | 13.588 | 3.76426 | 3.24E−11 |
| CELSR1 | cadherin_ EGF LAG seven-pass G-type | 13.4867 | 3.75347 | 6.06E−22 |
| | receptor 1 | | | |
| CCND2 | cyclin D2 | 13.4595 | 3.75055 | 6.45E−07 |
| LINC00951 | long intergenic non-protein coding RNA | 13.4392 | 3.74838 | 2.58E−06 |
| | 951 | | | |
| AZU1 | azurocidin 1 | 13.4366 | 3.7481 | 8.77E−10 |
| SULT1C2 | sulfotransferase family_ cytosolic_ 1C_ | 13.4353 | 3.74796 | 2.68E−06 |
| | member 2 | | | |
| LPAR4 | lysophosphatidic acid receptor 4 | 13.4176 | 3.74606 | 8.11E−12 |
| INA | internexin neuronal intermediate | 13.3079 | 3.73421 | 5.90E−76 |
| | filament protein_ alpha | | | |
| MYOZ3 | myozenin 3 | 13.1452 | 3.71646 | 6.32E−75 |
| AQP7P3 | aquaporin 7 pseudogene 3 | 13.0872 | 3.71009 | 3.62E−07 |
| FOXC1 | forkhead box C1 | 13.0634 | 3.70746 | 2.10E−53 |
| LRRC7 | leucine rich repeat containing 7 | 13.0529 | 3.7063 | 1.25E−08 |
| FZD3 | frizzled class receptor 3 | 13.0287 | 3.70362 | 7.46E−27 |
| NCALD | neurocalcin delta | 12.9782 | 3.69802 | 4.24E−13 |
| LSAMP- | LSAMP antisense RNA 1 | 12.9224 | 3.6918 | 3.76E−06 |
| AS1 | | | | |
| IRX4 | iroquois homeobox 4 | 12.8313 | 3.6816 | 1.79E−06 |
| PURG | purine-rich element binding protein G | 12.8256 | 3.68095 | 6.03E−10 |
| AMH | anti-Mullerian hormone | 12.7728 | 3.675 | 1.24E−21 |
| RIPPLY3 | ripply transcriptional repressor 3 | 12.6992 | 3.66667 | 2.22E−08 |
| LOC101927482 | uncharacterized LOC101927482 | 12.6952 | 3.66621 | 1.78E−15 |
| C1orf94 | chromosome 1 open reading frame 94 | 12.664 | 3.66266 | 8.39E−07 |
| FOXA1 | forkhead box A1 | 12.6375 | 3.65964 | 3.26E−07 |
| FSTL5 | follistatin-like 5 | 12.6272 | 3.65846 | 4.81E−08 |
| KCNJ2 | potassium channel_ inwardly rectifying | 12.5831 | 3.65341 | 1.16E−07 |
| | subfamily J_ member 2 | | | |
| XIRP1 | xin actin binding repeat containing 1 | 12.4469 | 3.63772 | 1.58E−06 |
| TMEM246 | transmembrane protein 246 | 12.3385 | 3.62509 | 2.04E−11 |
| LIPH | lipase_ member H | 12.2843 | 3.61874 | 5.47E−06 |
| AQP7P1 | aquaporin 7 pseudogene 1 | 12.1751 | 3.60586 | 2.31E−09 |
| CASKIN1 | CASK interacting protein 1 | 12.1053 | 3.59757 | 3.92E−15 |
| ACHE | acetylcholinesterase (Yt blood group) | 12.0642 | 3.59266 | 1.51E−17 |
| C14orf105 | chromosome 14 open reading frame 105 | 12.0171 | 3.58702 | 5.90E−06 |
| TNFRSF10C | tumor necrosis factor receptor | 11.8486 | 3.56665 | 8.74E−46 |
| | superfamily_ member 10c_ decoy | | | |
| | without an intracellular domain | | | |
| FAM43B | family with sequence similarity 43_ | 11.7047 | 3.54901 | 5.63E−10 |
| | member B | | | |
| CBLN2 | cerebellin 2 precursor | 11.6961 | 3.54795 | 9.91E−06 |
| FRZB | frizzled-related protein | 11.6693 | 3.54465 | 2.01E−47 |
| PTCHD4 | patched domain containing 4 | 11.6421 | 3.54128 | 6.59E−16 |
| DMRTA1 | DMRT-like family A1 | 11.6186 | 3.53836 | 6.83E−16 |
| ZSCAN1 | zinc finger and SCAN domain | 11.609 | 3.53717 | 4.18E−08 |
| | containing 1 | | | |
| EPHA7 | EPH receptor A7 | 11.5942 | 3.53533 | 3.06E−07 |
| GABRA4 | gamma-aminobutyric acid (GABA) A | 11.5797 | 3.53353 | 3.25E−06 |
| | receptor_ alpha 4 | | | |
| AFAP1L2 | actin filament associated protein 1-like 2 | 11.5445 | 3.52914 | 2.57E−11 |
| RAPIGAP2 | RAP1 GTPase activating protein 2 | 11.44 | 3.51602 | 4.39E−15 |
| CSDC2 | cold shock domain containing C2_ RNA | 11.4326 | 3.51508 | 5.86E−15 |
| | binding | | | |
| CGB8 | chorionic gonadotropin_beta | 11.3511 | 3.50476 | 1.28E−08 |
| | polypeptide 8 | | | |
| ARHGEF16 | Rho guanine nucleotide exchange factor | 11.3114 | 3.4997 | 2.42E−17 |
| | (GEF) 16 | | | |
| PCDH1 | protocadherin 1 | 11.2726 | 3.49475 | 1.68E−17 |
| NPPC | natriuretic peptide C | 11.2664 | 3.49396 | 1.28E−06 |
| ANGPTL4 | angiopoietin-like 4 | 11.2516 | 3.49206 | 4.55E−35 |
| ATP2B2 | ATPase_ Ca++ transporting_ plasma | 11.1335 | 3.47684 | 9.53E−10 |
| | membrane 2 | | | |
| RNF182 | ring finger protein 182 | 11.1267 | 3.47595 | 1.49E−10 |
| CCDC160 | coiled-coil domain containing 160 | 11.0566 | 3.46683 | 6.35E−12 |
| DACH2 | dachshund family transcription factor 2 | 11.0356 | 3.46409 | 2.10E−06 |
| PTPRN2 | protein tyrosine phosphatase_ receptor | 11.0305 | 3.46342 | 6.05E−07 |
| | type_ N polypeptide 2 | | | |
| IGFBP5 | insulin-like growth factor binding | 11.0117 | 3.46097 | 1.68E−06 |
| | protein 5 | | | |
| COL4A4 | collagen_ type IV_ alpha 4 | 11.0069 | 3.46033 | 6.39E−108 |
| TMEM74B | transmembrane protein 74B | 10.9141 | 3.44812 | 2.86E−09 |
| OCLN | occludin | 10.9085 | 3.44738 | 7.88E−18 |
| PTGIS | prostaglandin I2 (prostacyclin) synthase | 10.8909 | 3.44505 | 4.07E−17 |
| CIDEA | cell death-inducing DFFA-like effector a | 10.8757 | 3.44303 | 3.21E−07 |
| VANGL2 | VANGL planar cell polarity protein 2 | 10.8126 | 3.43464 | 2.88E−06 |
| DOCK8 | dedicator of cytokinesis 8 | 10.7905 | 3.43169 | 1.37E−06 |
| CCDC88C | coiled-coil domain containing 88C | 10.7762 | 3.42978 | 1.47E−08 |
| NMNAT3 | nicotinamide nucleotide | 10.7424 | 3.42524 | 1.21E−20 |
| | adenylyltransferase 3 | | | |
| TCF15 | transcription factor 15 (basic helix-loop- | 10.7253 | 3.42295 | 1.61E−07 |
| | helix) | | | |
| ITGB6 | integrin_ beta 6 | 10.6645 | 3.41475 | 7.14E−08 |
| SCUBE3 | signal peptide_CUB domain_ EGF-like | 10.622 | 3.40899 | 5.25E−30 |
| | 3 | | | |
| SOX17 | SRY (sex determining region Y)-box 17 | 10.6119 | 3.40761 | 2.79E−06 |
| IL2RB | interleukin 2 receptor_ beta | 10.6028 | 3.40637 | 7.12E−29 |
| ATCAY | ataxia_ cerebellar_ Cayman type | 10.5981 | 3.40573 | 8.13E−07 |
| FMN1 | formin 1 | 10.5973 | 3.40562 | 2.24E−36 |
| EFNA2 | ephrin-A2 | 10.5791 | 3.40314 | 1.56E−08 |
| MAGEB17 | melanoma antigen family B17 | 10.5548 | 3.39983 | 2.63E−06 |
| CERS1 | ceramide synthase 1 | 10.5375 | 3.39746 | 3.22E−09 |
| DLK1 | delta-like 1 homolog (Drosophila) | 10.5276 | 3.39611 | 5.93E−11 |
| DCHS1 | dachsous cadherin-related 1 | 10.5002 | 3.39234 | 2.11E−108 |
| SFTAIP | surfactant associated 1_ pseudogene | 10.3743 | 3.37494 | 1.05E−20 |
| FOXG1 | forkhead box G1 | 10.3464 | 3.37105 | 2.17E−05 |
| ADAMTS7P1 | ADAMTS7 pseudogene 1 | 10.3038 | 3.3651 | 2.14E−08 |
| LEMD1- | LEMD 1 antisense RNA 1 | 10.2887 | 3.36299 | 6.84E−08 |
| AS1 | | | | |
| C6orf141 | chromosome 6 open reading frame 141 | 10.1422 | 3.3423 | 1.99E−14 |
| EDN1 | endothelin 1 | 10.1099 | 3.3377 | 8.53E−77 |
| RAB9B | RAB9B_ member RAS oncogene family | 10.1049 | 3.33698 | 1.44E−32 |
| SLC29A2 | solute carrier family 29 (equilibrative | 10.0911 | 3.33501 | 3.03E−28 |
| | nucleoside transporter)_ member 2 | | | |
| GABRA5 | gamma-aminobutyric acid (GABA) A | 10.09 | 3.33486 | 8.12E−06 |
| | receptor_ alpha 5 | | | |
| RIMBP2 | RIMS binding protein 2 | 10.0289 | 3.32609 | 4.37E−06 |
| HTR1D | 5-hydroxytryptamine (serotonin) | 10.0216 | 3.32504 | 1.09E−17 |
| | receptor 1D_ G protein-coupled | | | |
| GAL3ST3 | galactose-3-O-sulfotransferase 3 | 9.99031 | 3.32053 | 4.84E−09 |
| OXTR | oxytocin receptor | 9.97952 | 3.31897 | 8.27E−11 |
| SESN3 | sestrin 3 | 9.97647 | 3.31853 | 8.95E−11 |
| CCDC148 | coiled-coil domain containing 148 | 9.92199 | 3.31063 | 1.51E−15 |
| EPHA5 | EPH receptor A5 | 9.84984 | 3.3001 | 4.99E−08 |
| CTTNBP2 | cortactin binding protein 2 | 9.84806 | 3.29984 | 1.31E−11 |
| NLRP10 | NLR family_ pyrin domain containing | 9.83285 | 3.29761 | 6.88E−08 |
| | 10 | | | |
| ANO4 | anoctamin 4 | 9.81113 | 3.29442 | 1.05E−10 |
| KLHL6 | kelch-like family member 6 | 9.78275 | 3.29024 | 4.00E−05 |
| ALPK3 | alpha-kinase 3 | 9.782 | 3.29013 | 1.59E−25 |
| THRB | thyroid hormone receptor_ beta | 9.77116 | 3.28853 | 3.43E−05 |
| TMEM63C | transmembrane protein 63C | 9.76168 | 3.28713 | 1.95E−42 |
| MLN | motilin | 9.75086 | 3.28553 | 5.19E−06 |
| LINC01082 | long intergenic non-protein coding RNA | 9.74789 | 3.28509 | 3.83E−05 |
| | 1082 | | | |
| GBX2 | gastrulation brain homeobox 2 | 9.63377 | 3.2681 | 1.35E−05 |
| PCYT1B | phosphate cytidylyltransferase 1_ | 9.59964 | 3.26298 | 8.46E−13 |
| | choline_ beta | | | |
| KRTAP4-9 | keratin associated protein 4-9 | 9.55569 | 3.25636 | 5.02E−05 |
| LOC90246 | uncharacterized LOC90246 | 9.53148 | 3.2527 | 1.42E−28 |
| PCDH19 | protocadherin 19 | 9.51511 | 3.25022 | 1.59E−09 |
| PCDHGB6 | protocadherin gamma subfamily B_ 6 | 9.49593 | 3.24731 | 1.93E−10 |
| FAM92B | family with sequence similarity 92_ | 9.43459 | 3.23796 | 1.44E−05 |
| | member B | | | |
| NTN4 | netrin 4 | 9.42825 | 3.23699 | 6.41E−12 |
| TPSG1 | tryptase gamma 1 | 9.3604 | 3.22657 | 4.47E−05 |
| PCDHA9 | protocadherin alpha 9 | 9.32536 | 3.22116 | 5.91E−05 |
| FAM110D | family with sequence similarity 110_ | 9.3129 | 3.21923 | 1.47E−44 |
| | member D | | | |
| GATA3 | GATA binding protein 3 | 9.25004 | 3.20946 | 2.07E−05 |
| ELN | elastin | 9.21082 | 3.20333 | 3.62E−29 |
| NTNG1 | netrin G1 | 9.15722 | 3.19491 | 6.47E−05 |
| VIP | vasoactive intestinal peptide | 9.13168 | 3.19088 | 6.52E−05 |
| LHX9 | LIM homeobox 9 | 9.10507 | 3.18667 | 8.09E−07 |
| MYOZ1 | myozenin 1 | 9.05729 | 3.17908 | 1.14E−07 |
| FAM84A | family with sequence similarity 84_ | 9.04907 | 3.17777 | 9.59E−08 |
| | member A | | | |
| APOE | apolipoprotein E | 9.04199 | 3.17664 | 2.45E−19 |
| LOC102723344 | uncharacterized LOC102723344 | 9.02815 | 3.17443 | 6.68E−08 |
| RUNDC3B | RUN domain containing 3B | 8.95349 | 3.16245 | 6.26E−06 |
| C5orf46 | chromosome 5 open reading frame 46 | 8.91071 | 3.15554 | 1.80E−27 |
| LYVE1 | lymphatic vessel endothelial hyaluronan | 8.90262 | 3.15423 | 7.29E−06 |
| | receptor 1 | | | |
| LINC00547 | long intergenic non-protein coding RNA | 8.88271 | 3.151 | 5.29E−05 |
| | 547 | | | |
| SPINT2 | serine peptidase inhibitor_ Kunitz type_ | 8.85505 | 3.1465 | 2.28E−25 |
| | 2 | | | |
| GDF6 | growth differentiation factor 6 | 8.82209 | 3.14112 | 1.43E−27 |
| DACH1 | dachshund family transcription factor 1 | 8.79004 | 3.13587 | 9.43E−05 |
| HAP1 | huntingtin-associated protein 1 | 8.77075 | 3.1327 | 5.15E−12 |
| LOC149684 | uncharacterized LOC149684 | 8.74792 | 3.12894 | 2.96E−06 |
| BMP3 | bone morphogenetic protein 3 | 8.74477 | 3.12842 | 1.07E−08 |
| ALDH5A1 | aldehyde dehydrogenase 5 family_ | 8.72285 | 3.1248 | 1.65E−24 |
| | member A1 | | | |
| KIAA1211 | KIAA1211 | 8.72013 | 3.12435 | 9.95E−10 |
| MAP3K7CL | MAP3K7 C-terminal like | 8.71361 | 3.12327 | 1.40E−59 |
| AQP5 | aquaporin 5 | 8.67359 | 3.11663 | 1.39E−06 |
| LINC00887 | long intergenic non-protein coding RNA | 8.67191 | 3.11635 | 5.11E−07 |
| | 887 | | | |
| ACSM4 | acyl-CoA synthetase medium-chain | 8.67101 | 3.1162 | 3.26E−05 |
| | family member 4 | | | |
| SLC12A5 | solute carrier family 12 | 8.66692 | 3.11552 | 1.95E−16 |
| | (potassium/chloride transporter)_ | | | |
| | member 5 | | | |
| PPP1R14A | protein phosphatase 1_ regulatory | 8.62037 | 3.10775 | 6.73E−17 |
| | (inhibitor) subunit 14A | | | |
| KCNMB1 | potassium channel subfamily M | 8.61667 | 3.10713 | 2.55E−10 |
| | regulatory beta subunit 1 | | | |
| SLC5A4 | solute carrier family 5 (glucose activated | 8.5971 | 3.10385 | 4.58E−06 |
| | ion channel)_ member 4 | | | |
| ZNF423 | zinc finger protein 423 | 8.58459 | 3.10175 | 5.59E−15 |
| CHRNA7 | cholinergic receptor_ nicotinic_ alpha 7 | 8.57015 | 3.09932 | 1.97E−05 |
| | (neuronal) | | | |
| FGF11 | fibroblast growth factor 11 | 8.53766 | 3.09384 | 2.45E−58 |
| CYTL1 | cytokine-like 1 | 8.52908 | 3.09239 | 4.27E−35 |
| GPR20 | G protein-coupled receptor 20 | 8.52654 | 3.09196 | 1.94E−09 |
| LOC100507600 | uncharacterized LOC100507600 | 8.52406 | 3.09154 | 7.30E−09 |
| SERTAD4 | SERTA domain containing 4 | 8.52388 | 3.09151 | 5.96E−15 |
| PROC | protein C (inactivator of coagulation | 8.49091 | 3.08592 | 3.83E−07 |
| | factors Va and VIIIa) | | | |
| JAM2 | junctional adhesion molecule 2 | 8.48856 | 3.08552 | 7.86E−12 |
| PCDHB16 | protocadherin beta 16 | 8.48268 | 3.08452 | 7.41E−18 |
| GRIK1- | GRIK1 antisense RNA 1 | 8.45011 | 3.07897 | 2.75E−05 |
| AS1 | | | | |
| CGB2 | chorionic gonadotropin_ beta | 8.43343 | 3.07612 | 0.00012 |
| | polypeptide 2 | | | |
| CDH8 | cadherin 8_type 2 | 8.4109 | 3.07226 | 8.44E−05 |
| GPLD1 | glycosylphosphatidylinositol specific | 8.40962 | 3.07204 | 5.72E−12 |
| | phospholipase D1 | | | |
| ZNF521 | zinc finger protein 521 | 8.40507 | 3.07126 | 9.51E−26 |
| FAM83E | family with sequence similarity 83_ | 8.38046 | 3.06703 | 6.69E−06 |
| | member E | | | |
| SBK3 | SH3 domain binding kinase family_ | 8.31767 | 3.05618 | 7.17E−05 |
| | member 3 | | | |
| WT1 | Wilms tumor 1 | 8.30932 | 3.05473 | 4.77E−05 |
| HID 1 | HID1 domain containing | 8.25518 | 3.0453 | 1.09E−27 |
| ERC2 | ELKS/RAB6-interacting/CAST family | 8.21762 | 3.03872 | 1.91E−06 |
| | member 2 | | | |
| ESPN | espin | 8.21625 | 3.03848 | 2.65E−06 |
| WT1-AS | WT1 antisense RNA | 8.19435 | 3.03463 | 6.39E−05 |
| APBB1IP | amyloid beta (A4) precursor protein- | 8.191 | 3.03404 | 8.22E−12 |
| | binding_ family B_ member 1 | | | |
| | interacting protein | | | |
| PIEZO2 | piezo-type mechanosensitive ion channel | 8.18754 | 3.03343 | 4.71E−09 |
| | component 2 | | | |
| AC093375.1 | NA | 8.15554 | 3.02778 | 0.000116 |
| POTEF | POTE ankyrin domain family_ member | 8.1373 | 3.02455 | 1.74E−28 |
| | F | | | |
| JSRP1 | junctional sarcoplasmic reticulum | 8.12772 | 3.02285 | 4.78E−06 |
| | protein 1 | | | |
| DRD1 | dopamine receptor D1 | 8.11798 | 3.02112 | 5.53E−05 |
| SYT9 | synaptotagmin IX | 8.04426 | 3.00796 | 7.24E−06 |
| KRT7 | keratin 7_ type II | 8.02866 | 3.00516 | 1.44E−64 |
| LINC00858 | long intergenic non-protein coding RNA | 8.00705 | 3.00127 | 0.000153 |
| | 858 | | | |
| ABCA13 | ATP-binding cassette_ sub-family A | 7.98465 | 2.99723 | 3.12E−07 |
| | (ABC1)_ member 13 | | | |
| IGF1 | insulin-like growth factor 1 | 7.97768 | 2.99597 | 0.000181 |
| | (somatomedin C) | | | |
| PALD1 | phosphatase domain containing_paladin | 7.96188 | 2.99311 | 1.28E−08 |
| | 1 | | | |
| SOWAHB | sosondowah ankyrin repeat domain | 7.95753 | 2.99232 | 3.29E−05 |
| | family member B | | | |
| TMEM35 | transmembrane protein 35 | 7.92494 | 2.9864 | 1.78E−38 |
| ACTC1 | actin_ alpha_ cardiac muscle 1 | 7.9239 | 2.98621 | 3.75E−16 |
| CACNG6 | calcium channel_ voltage-dependent_ | 7.87156 | 2.97665 | 5.54E−34 |
| | gamma subunit 6 | | | |
| PPL | periplakin | 7.86982 | 2.97633 | 1.32E−14 |
| TRPC5OS | TRPC5 opposite strand | 7.86845 | 2.97608 | 0.00015 |
| ABLIM1 | actin binding LIM protein 1 | 7.82169 | 2.96748 | 2.65E−19 |
| FOXL2 | forkhead box L2 | 7.82115 | 2.96738 | 8.54E−08 |
| TMOD1 | tropomodulin 1 | 7.7731 | 2.95849 | 0.000143 |
| FOXE3 | forkhead box E3 | 7.72208 | 2.94899 | 5.15E−05 |
| LINC00890 | long intergenic non-protein coding RNA | 7.70423 | 2.94565 | 9.71E−23 |
| | 890 | | | |
| PLN | phospholamban | 7.69819 | 2.94452 | 3.81E−08 |
| CAPN11 | calpain 11 | 7.69782 | 2.94445 | 4.09E−08 |
| MAGEL2 | melanoma antigen family L2 | 7.68668 | 2.94236 | 5.71E−12 |
| LINC00622 | long intergenic non-protein coding RNA | 7.67023 | 2.93927 | 4.03E−13 |
| | 622 | | | |
| RASL10A | RAS-like_ family 10_ member A | 7.61561 | 2.92896 | 4.24E−05 |
| C11orf87 | chromosome 11 open reading frame 87 | 7.60006 | 2.92601 | 2.64E−06 |
| LINC00840 | long intergenic non-protein coding RNA | 7.5575 | 2.91791 | 3.57E−06 |
| | 840 | | | |
| SCG2 | secretogranin II | 7.55457 | 2.91735 | 7.95E−22 |
| PXDNL | peroxidasin-like | 7.5359 | 2.91378 | 8.38E−06 |
| RASSF2 | Ras association (RalGDS/AF-6) domain | 7.49563 | 2.90605 | 3.11E−57 |
| | family member 2 | | | |
| VGF | VGF nerve growth factor inducible | 7.4909 | 2.90514 | 1.11E−12 |
| NLGN1 | neuroligin 1 | 7.46018 | 2.89921 | 7.92E−06 |
| GRPR | gastrin-releasing peptide receptor | 7.45051 | 2.89734 | 8.69E−29 |
| ARL14 | ADP-ribosylation factor-like 14 | 7.44819 | 2.89689 | 1.18E−05 |
| RENBP | renin binding protein | 7.40233 | 2.88798 | 2.31E−06 |
| TRIML2 | tripartite motif family-like 2 | 7.37534 | 2.88271 | 0.00029 |
| FGD4 | FYVE_ RhoGEF and PH domain | 7.33934 | 2.87565 | 1.76E−20 |
| | containing 4 | | | |
| BIRC7 | baculoviral IAP repeat containing 7 | 7.33522 | 2.87484 | 0.000137 |
| CADM4 | cell adhesion molecule 4 | 7.33024 | 2.87386 | 9.04E−38 |
| ANKS1B | ankyrin repeat and sterile alpha motif | 7.32308 | 2.87245 | 1.49E−05 |
| | domain containing 1B | | | |
| LOC100130899 | uncharacterized LOC100130899 | 7.31958 | 2.87176 | 0.00027 |
| DCDC2 | doublecortin domain containing 2 | 7.30194 | 2.86828 | 0.000282 |
| CD101 | CD101 molecule | 7.30179 | 2.86825 | 3.82E−06 |
| KIF21B | kinesin family member 21B | 7.29992 | 2.86788 | 1.17E−68 |
| EEF1A2 | eukaryotic translation elongation factor | 7.24763 | 2.85751 | 1.16E−22 |
| | 1 alpha 2 | | | |
| CASC15 | cancer susceptibility candidate 15 (non- | 7.23774 | 2.85554 | 6.66E−07 |
| | protein coding) | | | |
| DCLK1 | doublecortin-like kinase 1 | 7.22426 | 2.85285 | 2.25E−15 |
| SOX18 | SRY (sex determining region Y)-box 18 | 7.2107 | 2.85014 | 1.24E−08 |
| CTNND2 | catenin (cadherin-associated protein)_ | 7.20676 | 2.84935 | 0.000304 |
| | delta 2 | | | |
| NHS | Nance-Horan syndrome (congenital | 7.20476 | 2.84895 | 5.32E−08 |
| | cataracts and dental anomalies) | | | |
| LOC100128531 | uncharacterized LOC100128531 | 7.18551 | 2.84509 | 0.000395 |
| RGS9 | regulator of G-protein signaling 9 | 7.17555 | 2.84309 | 2.06E−15 |
| NCF2 | neutrophil cytosolic factor 2 | 7.16358 | 2.84068 | 1.39E−06 |
| LINC00649 | long intergenic non-protein coding RNA | 7.15772 | 2.8395 | 2.36E−08 |
| | 649 | | | |
| PCDHB8 | protocadherin beta 8 | 7.13874 | 2.83567 | 7.07E−05 |
| CLEC4GP1 | C-type lectin domain family 4_ member | 7.12243 | 2.83237 | 0.000363 |
| | G pseudogene 1 | | | |
| PCBP3 | poly(rC) binding protein 3 | 7.12199 | 2.83228 | 1.76E−13 |
| CSMD3 | CUB and Sushi multiple domains 3 | 7.11932 | 2.83174 | 5.68E−13 |
| SERPINA9 | serpin peptidase inhibitor_ clade A | 7.01897 | 2.81126 | 0.000456 |
| | (alpha-1 antiproteinase_ antitrypsin)_ | | | |
| | member 9 | | | |
| ELAVL2 | ELAV like neuron-specific RNA | 7.01328 | 2.81009 | 8.89E−05 |
| | binding protein 2 | | | |
| LBH | limb bud and heart development | 6.9804 | 2.80331 | 5.82E−57 |
| KCNN2 | potassium channel_ calcium activated | 6.96663 | 2.80046 | 4.14E−14 |
| | intermediate/small conductance | | | |
| | subfamily N alpha_ member 2 | | | |
| SEMA3F | sema domain_ immunoglobulin domain | 6.94267 | 2.79549 | 1.50E−60 |
| | (Ig)_ short basic domain_ secreted_ | | | |
| | (semaphorin) 3F | | | |
| BEND5 | BEN domain containing 5 | 6.89538 | 2.78563 | 0.000319 |
| P2RX6P | purinergic receptor P2X_ ligand gated | 6.89509 | 2.78557 | 1.13E−07 |
| | ion channel_6 pseudogene | | | |
| LRMP | lymphoid-restricted membrane protein | 6.89452 | 2.78545 | 9.07E−08 |
| CNTNAP3B | contactin associated protein-like 3B | 6.89089 | 2.78469 | 0.000117 |
| ZCCHC18 | zinc finger_ CCHC domain containing | 6.88597 | 2.78366 | 2.72E−16 |
| | 18 | | | |
| RASSF10 | Ras association (RalGDS/AF-6) domain | 6.87391 | 2.78113 | 0.000516 |
| | family (N-terminal) member 10 | | | |
| ZIC2 | Zic family member 2 | 6.86139 | 2.7785 | 0.000395 |
| CYGB | cytoglobin | 6.84267 | 2.77456 | 3.13E−31 |
| TYROBP | TYRO protein tyrosine kinase binding | 6.83154 | 2.77221 | 0.00043 |
| | protein | | | |
| LOC100507006 | uncharacterized LOC100507006 | 6.81583 | 2.76889 | 0.000275 |
| THSD7A | thrombospondin_ type I_ domain | 6.81361 | 2.76842 | 1.62E−06 |
| | containing 7A | | | |
| MIR4321 | microRNA 4321 | 6.7899 | 2.76339 | 0.000527 |
| DYSF | dysferlin | 6.78731 | 2.76284 | 6.21E−23 |
| SYTL1 | synaptotagmin-like 1 | 6.7575 | 2.75649 | 1.41E−09 |
| ADCY10P1 | adenylate cyclase 10 (soluble) | 6.74884 | 2.75464 | 2.20E−09 |
| | pseudogene 1 | | | |
| JAG2 | jagged 2 | 6.73398 | 2.75146 | 1.38E−15 |
| COL6A5 | collagen_ type VI_ alpha 5 | 6.72302 | 2.74911 | 0.000601 |
| PAQR6 | progestin and adipoQ receptor family | 6.72195 | 2.74888 | 1.92E−09 |
| | member VI | | | |
| FSTL4 | follistatin-like 4 | 6.71483 | 2.74735 | 0.000619 |
| UPB1 | ureidopropionase_ beta | 6.71129 | 2.74659 | 1.91E−06 |
| LZTS1 | leucine zipper_ putative tumor | 6.70896 | 2.74609 | 9.30E−74 |
| | suppressor 1 | | | |
| CNGA1 | cyclic nucleotide gated channel alpha 1 | 6.70645 | 2.74555 | 1.88E−05 |
| KCNH1 | potassium channel_ voltage gated eag | 6.69549 | 2.74319 | 1.61E−09 |
| | related subfamily H_ member 1 | | | |
| RGPD1 | RANBP2-like and GRIP domain | 6.66165 | 2.73588 | 9.04E−06 |
| | containing 1 | | | |
| SPINK1 | serine peptidase inhibitor_ Kazal type 1 | 6.66082 | 2.7357 | 0.000649 |
| ECSCR | endothelial cell surface expressed | 6.65985 | 2.73549 | 3.50E−17 |
| | chemotaxis and apoptosis regulator | | | |
| MYL4 | myosin_ light chain 4_ alkali; atrial_ | 6.65547 | 2.73454 | 4.43E−07 |
| | embryonic | | | |
| ADCY4 | adenylate cyclase 4 | 6.64339 | 2.73192 | 1.28E−05 |
| ZMAT4 | zinc finger_ matrin-type 4 | 6.62004 | 2.72684 | 6.04E−10 |
| DUSP15 | dual specificity phosphatase 15 | 6.59183 | 2.72068 | 0.000655 |
| SHROOM2 | shroom family member 2 | 6.57527 | 2.71705 | 1.20E−61 |
| RAPGEF5 | Rap guanine nucleotide exchange factor | 6.54952 | 2.71139 | 9.33E−06 |
| | (GEF) 5 | | | |
| CTAGE6 | CTAGE family_ member 6 | 6.54925 | 2.71133 | 0.00016 |
| Clorf106 | chromosome 1 open reading frame 106 | 6.53003 | 2.70709 | 6.55E−41 |
| TIE1 | tyrosine kinase with immunoglobulin- | 6.49856 | 2.70012 | 4.16E−36 |
| | like and EGF-like domains 1 | | | |
| GZMA | granzyme A (granzyme 1_ cytotoxic T- | 6.47226 | 2.69427 | 0.000623 |
| | lymphocyte-associated serine esterase 3) | | | |
| RHOV | ras homolog family member V | 6.46433 | 2.6925 | 1.72E−06 |
| LINC01002 | long intergenic non-protein coding RNA | 6.42043 | 2.68267 | 2.59E−05 |
| | 1002 | | | |
| LEPREL1 | prolyl 3-hydroxylase 2 | 6.40243 | 2.67862 | 8.05E−55 |
| KRTAP5-1 | keratin associated protein 5-1 | 6.40017 | 2.67811 | 0.000735 |
| TLR2 | toll-like receptor 2 | 6.37577 | 2.6726 | 0.000277 |
| PALM2 | paralemmin 2 | 6.34878 | 2.66648 | 3.95E−17 |
| LINC00704 | long intergenic non-protein coding RNA | 6.317 | 2.65924 | 7.01E−06 |
| | 704 | | | |
| LOC100652824 | NA | 6.31166 | 2.65802 | 3.63E−11 |
| AADACP1 | arylacetamide deacetylase pseudogene 1 | 6.301 | 2.65558 | 0.000388 |
| TLL2 | tolloid-like 2 | 6.29999 | 2.65535 | 2.58E−12 |
| ENTPD3 | ectonucleoside triphosphate | 6.28582 | 2.6521 | 4.00E−05 |
| | diphosphohydrolase 3 | | | |
| ATRNL1 | attractin-like 1 | 6.28468 | 2.65184 | 2.86E−06 |
| LINC01239 | long intergenic non-protein coding RNA | 6.2672 | 2.64782 | 1.04E−05 |
| | 1239 | | | |
| ZIC1 | Zic family member 1 | 6.26702 | 2.64778 | 0.000977 |
| UPK1A | uroplakin 1A | 6.24976 | 2.6438 | 2.79E−05 |
| LOC100507534 | uncharacterized LOC100507534 | 6.2293 | 2.63907 | 1.20E−05 |
| PDZD2 | PDZ domain containing 2 | 6.22718 | 2.63858 | 3.38E−32 |
| SEMA6B | sema domain_ transmembrane domain | 6.21709 | 2.63624 | 9.15E−14 |
| | (TM)_ and cytoplasmic domain_ | | | |
| | (semaphorin) 6B | | | |
| MEGF10 | multiple EGF-like-domains 10 | 6.21063 | 2.63474 | 0.000763 |
| LINC01197 | long intergenic non-protein coding RNA | 6.20461 | 2.63334 | 0.000813 |
| | 1197 | | | |
| SPATA31E1 | SPATA31 subfamily E_ member 1 | 6.19447 | 2.63098 | 0.000662 |
| A2M-AS1 | A2M antisense RNA 1 (head to head) | 6.19176 | 2.63035 | 2.13E−05 |
| CECR2 | cat eye syndrome chromosome region_ | 6.19017 | 2.62998 | 0.000364 |
| | candidate 2 | | | |
| DNAH8 | dynein_ axonemal_ heavy chain 8 | 6.18036 | 2.62769 | 0.000829 |
| GPR183 | G protein-coupled receptor 183 | 6.17916 | 2.62741 | 2.62E−18 |
| PRICKLE1 | prickle homolog 1 | 6.17192 | 2.62572 | 3.61E−10 |
| MEI4 | meiotic double-stranded break formation | 6.16504 | 2.62411 | 0.000297 |
| | protein 4 | | | |
| GNAO1 | guanine nucleotide binding protein (G | 6.16397 | 2.62386 | 1.09E−18 |
| | protein)_ alpha activating activity | | | |
| | polypeptide O | | | |
| PCDHA2 | protocadherin alpha 2 | 6.15846 | 2.62257 | 0.000939 |
| FGFBP3 | fibroblast growth factor binding protein | 6.15599 | 2.62199 | 1.51E− |
| | 3 | | | 120 |
| PTPN7 | protein tyrosine phosphatase_ non- | 6.13465 | 2.61698 | 9.90E−05 |
| | receptor type 7 | | | |
| BAALC | brain and acute leukemia_ cytoplasmic | 6.12976 | 2.61583 | 5.89E−17 |
| ZFHX2 | zinc finger homeobox 2 | 6.12963 | 2.6158 | 1.58E−11 |
| LAMC2 | laminin_ gamma 2 | 6.12581 | 2.6149 | 6.63E−12 |
| PPARG | peroxisome proliferator-activated | 6.12428 | 2.61454 | 2.53E−11 |
| | receptor gamma | | | |
| LOC729737 | uncharacterized LOC729737 | 6.11478 | 2.6123 | 3.85E−11 |
| RASGRF1 | Ras protein-specific guanine nucleotide- | 6.1072 | 2.61051 | 8.10E−24 |
| | releasing factor 1 | | | |
| ACVR1C | activin A receptor_ type IC | 6.08514 | 2.60529 | 1.32E−07 |
| ST6GAL2 | ST6 beta-galactosamide alpha-2_6- | 6.08295 | 2.60477 | 1.66E−19 |
| | sialyltranferase 2 | | | |
| FAM162B | family with sequence similarity 162_ | 6.08193 | 2.60453 | 0.000742 |
| | member B | | | |
| MYOZ2 | myozenin 2 | 6.07679 | 2.60331 | 2.14E−10 |
| ZIC5 | Zic family member 5 | 6.06955 | 2.60159 | 0.000829 |
| SLC7A9 | solute carrier family 7 (amino acid | 6.05199 | 2.59741 | 5.05E−07 |
| | transporter light chain_ bo_+ system)_ | | | |
| | member 9 | | | |
| GPR143 | G protein-coupled receptor 143 | 6.04486 | 2.59571 | 1.54E−18 |
| WNT16 | wingless-type MMTV integration site | 6.03971 | 2.59448 | 4.60E−08 |
| | family_ member 16 | | | |
| LINC00222 | long intergenic non-protein coding RNA | 6.03009 | 2.59218 | 0.000545 |
| | 222 | | | |
| PIFO | primary cilia formation | 6.0247 | 2.59089 | 1.19E−09 |
| MDFI | MyoD family inhibitor | 6.02437 | 2.59081 | 4.67E−15 |
| SGIP1 | SH3-domain GRB2-like (endophilin) | 6.0134 | 2.58818 | 1.29E−15 |
| | interacting protein 1 | | | |
| FSIP2 | fibrous sheath interacting protein 2 | 6.0124 | 2.58794 | 4.68E−06 |
| ACAN | aggrecan | 6.00973 | 2.5873 | 1.06E−08 |
| LOC400863 | NA | 6.00885 | 2.58709 | 7.71E−05 |
| C11orf88 | chromosome 11 open reading frame 88 | 5.97741 | 2.57952 | 9.46E−15 |
| TSPAN18 | tetraspanin 18 | 5.96437 | 2.57637 | 9.71E−07 |
| VSTM2L | V-set and transmembrane domain | 5.96346 | 2.57615 | 1.08E−12 |
| | containing 2 like | | | |
| LINC00460 | long intergenic non-protein coding RNA | 5.95206 | 2.57339 | 6.68E−09 |
| | 460 | | | |
| HOXB8 | homeobox B8 | 5.94534 | 2.57176 | 7.30E−22 |
| LINC00086 | small integral membrane protein 10 like | 5.9242 | 2.56662 | 1.98E−08 |
| | 2A | | | |
| CDHR1 | cadherin-related family member 1 | 5.92223 | 2.56614 | 0.001148 |
| BMF | Bcl2 modifying factor | 5.92087 | 2.56581 | 2.33E−12 |
| RUNX3 | runt-related transcription factor 3 | 5.91292 | 2.56387 | 0.00036 |
| SCN5A | sodium channel_ voltage gated_ type V | 5.88809 | 2.5578 | 8.62E−07 |
| | alpha subunit | | | |
| GLRA4 | glycine receptor_ alpha 4 | 5.88793 | 2.55776 | 0.001253 |
| PTPRR | protein tyrosine phosphatase_ receptor | 5.87154 | 2.55374 | 1.05E−06 |
| | type_ R | | | |
| NTF4 | neurotrophin 4 | 5.87053 | 2.55349 | 3.68E−12 |
| MCF2 | MCF.2 cell line derived transforming | 5.86829 | 2.55294 | 2.85E−05 |
| | sequence | | | |
| TF | transferrin | 5.84908 | 2.54821 | 3.31E−06 |
| ATP2B3 | ATPase_ Ca++ transporting_ plasma | 5.84714 | 2.54773 | 1.90E−06 |
| | membrane 3 | | | |
| CD37 | CD37 molecule | 5.83422 | 2.54454 | 7.82E−09 |
| LAPTM5 | lysosomal protein transmembrane 5 | 5.82008 | 2.54104 | 3.89E−31 |
| RAMP2- | RAMP2 antisense RNA 1 | 5.81049 | 2.53866 | 1.81E−09 |
| AS1 | | | | |
| IGLON5 | IgLON family member 5 | 5.80401 | 2.53705 | 6.67E−05 |
| SLC6A17 | solute carrier family 6 (neutral amino | 5.79641 | 2.53516 | 3.43E−15 |
| | acid transporter)_ member 17 | | | |
| GIPC3 | GIPC PDZ domain containing family_ | 5.7752 | 2.52987 | 6.08E−59 |
| | member 3 | | | |
| ASXL3 | additional sex combs like transcriptional | 5.77324 | 2.52938 | 0.000148 |
| | regulator 3 | | | |
| PCDHAC1 | protocadherin alpha subfamily C_ 1 | 5.77243 | 2.52918 | 0.001215 |
| GRIK4 | glutamate receptor_ ionotropic_ kainate | 5.77079 | 2.52877 | 8.42E−06 |
| | 4 | | | |
| IRF6 | interferon regulatory factor 6 | 5.7632 | 2.52687 | 8.02E−09 |
| KRT23 | keratin 23_ type I | 5.75905 | 2.52583 | 2.56E−07 |
| ST6GALNAC1 | ST6 (alpha-N-acetyl-neuraminyl-2_3- | 5.75581 | 2.52502 | 0.001081 |
| | beta-galactosyl-1_3)-N- | | | |
| | acetylgalactosaminide alpha-2_6- | | | |
| | sialyltransferase 1 | | | |
| CYP2E1 | cytochrome P450_ family 2_ subfamily | 5.7484 | 2.52316 | 5.32E−07 |
| | E_ polypeptide 1 | | | |
| SIPA1L2 | signal-induced proliferation-associated 1 | 5.74103 | 2.52131 | 4.32E−53 |
| | like 2 | | | |
| CACNA2D3 | calcium channel_ voltage-dependent_ | 5.73638 | 2.52014 | 2.26E−05 |
| | alpha 2/delta subunit 3 | | | |
| CCDC3 | coiled-coil domain containing 3 | 5.72514 | 2.51731 | 1.02E−07 |
| PTGS1 | prostaglandin-endoperoxide synthase 1 | 5.72137 | 2.51636 | 6.83E−12 |
| | (prostaglandin G/H synthase and | | | |
| | cyclooxygenase) | | | |
| RGS7 | regulator of G-protein signaling 7 | 5.71709 | 2.51528 | 1.57E−07 |
| LINC01260 | long intergenic non-protein coding RNA | 5.71614 | 2.51504 | 0.00024 |
| | 1260 | | | |
| LOC102724849 | uncharacterized LOC102724849 | 5.71392 | 2.51448 | 2.09E−07 |
| LRRN4 | leucine rich repeat neuronal 4 | 5.71217 | 2.51404 | 4.42E−15 |
| SP140 | SP140 nuclear body protein | 5.71063 | 2.51365 | 6.75E−14 |
| C19orf81 | chromosome 19 open reading frame 81 | 5.70956 | 2.51338 | 4.55E−05 |
| KLHL4 | kelch-like family member 4 | 5.69849 | 2.51058 | 1.19E−08 |
| CD163L1 | CD163 molecule-like 1 | 5.67893 | 2.50562 | 8.29E−08 |
| TUBA3E | tubulin_ alpha 3e | 5.66777 | 2.50278 | 0.001189 |
| FGF13 | fibroblast growth factor 13 | 5.66568 | 2.50225 | 0.00092 |
| GSC | goosecoid homeobox | 5.6614 | 2.50116 | 1.08E−05 |
| CGB5 | chorionic gonadotropin_ beta | 5.64166 | 2.49612 | 0.000597 |
| | polypeptide 5 | | | |
| PCDHB5 | protocadherin beta 5 | 5.63693 | 2.49491 | 8.16E−06 |
| SRCRB4D | scavenger receptor cysteine rich family_ | 5.63486 | 2.49438 | 2.65E−19 |
| | 4 domains | | | |
| ZAP70 | zeta-chain (TCR) associated protein | 5.62596 | 2.4921 | 0.000113 |
| | kinase 70 kDa | | | |
| CCDC81 | coiled-coil domain containing 81 | 5.6037 | 2.48638 | 6.86E−14 |
| KIAA1456 | KIAA1456 | 5.60048 | 2.48555 | 1.55E−20 |
| NFATC2 | nuclear factor of activated T-cells_ | 5.59757 | 2.4848 | 1.37E−19 |
| | cytoplasmic_ calcineurin-dependent 2 | | | |
| MUC19 | mucin 19_ oligomeric | 5.59536 | 2.48423 | 0.000489 |
| KCNJ6 | potassium channel_ inwardly rectifying | 5.59284 | 2.48358 | 0.001973 |
| | subfamily J_ member 6 | | | |
| MTRNR2L10 | MT-RNR2-like 10 | 5.58513 | 2.48159 | 9.04E−05 |
| ZBTB46 | zinc finger and BTB domain containing | 5.57495 | 2.47896 | 1.21E−23 |
| | 46 | | | |
| PCDHB14 | protocadherin beta 14 | 5.56515 | 2.47642 | 2.03E−06 |
| IGSF3 | immunoglobulin superfamily_ member 3 | 5.56368 | 2.47604 | 1.39E−14 |
| NOVA2 | neuro-oncological ventral antigen 2 | 5.5601 | 2.47511 | 4.01E−06 |
| DRP2 | dystrophin related protein 2 | 5.54662 | 2.47161 | 1.49E−37 |
| PRTG | protogenin | 5.53752 | 2.46924 | 1.84E−09 |
| KIF26A | kinesin family member 26A | 5.53717 | 2.46915 | 3.41E−08 |
| LINC01013 | long intergenic non-protein coding RNA | 5.52759 | 2.46665 | 0.000302 |
| | 1013 | | | |
| KNDC1 | kinase non-catalytic C-lobe domain | 5.52062 | 2.46483 | 1.87E−08 |
| | (KIND) containing 1 | | | |
| PAK3 | p21 protein (Cdc42/Rac)-activated | 5.51324 | 2.4629 | 5.54E−32 |
| | kinase 3 | | | |
| TMEM52B | transmembrane protein 52B | 5.51278 | 2.46278 | 0.002149 |
| HOXB13 | homeobox B13 | 5.48937 | 2.45664 | 1.06E−05 |
| COL23A1 | collagen_ type XXIII_ alpha 1 | 5.4837 | 2.45515 | 0.001602 |
| DNM3 | dynamin 3 | 5.48298 | 2.45496 | 5.19E−11 |
| PAX9 | paired box 9 | 5.46356 | 2.44984 | 0.002241 |
| SETBP1 | SET binding protein 1 | 5.45784 | 2.44833 | 5.88E−06 |
| FGF16 | fibroblast growth factor 16 | 5.4399 | 2.44358 | 0.002308 |
| DUSP26 | dual specificity phosphatase 26 | 5.43809 | 2.4431 | 0.002181 |
| | (putative) | | | |
| BEX2 | brain expressed X-linked 2 | 5.43259 | 2.44164 | 1.45E−09 |
| FAM84B | family with sequence similarity 84_ | 5.4242 | 2.43941 | 3.53E−13 |
| | member B | | | |
| SDK2 | sidekick cell adhesion molecule 2 | 5.40851 | 2.43523 | 1.06E−10 |
| KBTBD11 | kelch repeat and BTB (POZ) domain | 5.40671 | 2.43475 | 1.16E−10 |
| | containing 11 | | | |
| GRHL3 | grainyhead-like transcription factor 3 | 5.40217 | 2.43354 | 8.32E−07 |
| ZBED2 | zinc finger_ BED-type containing 2 | 5.40094 | 2.43321 | 9.10E−07 |
| TMC8 | transmembrane channel-like 8 | 5.3717 | 2.42538 | 0.001867 |
| C2CD4C | C2 calcium-dependent domain | 5.37103 | 2.4252 | 1.38E−10 |
| | containing 4C | | | |
| NBL1 | neuroblastoma 1_ DAN family BMP | 5.3578 | 2.42164 | 0.000893 |
| | antagonist | | | |
| ARL4C | ADP-ribosylation factor-like 4C | 5.34848 | 2.41913 | 5.53E−19 |
| MS4A4A | membrane-spanning 4-domains_ | 5.33327 | 2.41502 | 0.001852 |
| | subfamily A_ member 4A | | | |
| GLB1L2 | galactosidase_ beta 1-like 2 | 5.32363 | 2.41241 | 3.97E−12 |
| FAM131B | family with sequence similarity 131_ | 5.31858 | 2.41104 | 1.09E−19 |
| | member B | | | |
| LOC643542 | uncharacterized LOC643542 | 5.31821 | 2.41094 | 0.00023 |
| TMEM151B | transmembrane protein 151B | 5.30974 | 2.40864 | 0.002088 |
| LMO2 | LIM domain only 2 (rhombotin-like 1) | 5.30653 | 2.40777 | 0.000314 |
| IGDCC4 | immunoglobulin superfamily_ DCC | 5.29724 | 2.40524 | 5.68E−72 |
| | subclass_ member 4 | | | |
| OPCML | opioid binding protein/cell adhesion | 5.28436 | 2.40173 | 5.06E−05 |
| | molecule-like | | | |
| CACNG8 | calcium channel_ voltage-dependent_ | 5.27975 | 2.40047 | 1.83E−16 |
| | gamma subunit 8 | | | |
| RORB | RAR-related orphan receptor B | 5.24415 | 2.39071 | 0.002129 |
| HAND1 | heart and neural crest derivatives | 5.22167 | 2.38451 | 1.82E−06 |
| | expressed 1 | | | |
| SULT4A1 | sulfotransferase family 4A_ member 1 | 5.21208 | 2.38186 | 1.47E−06 |
| HLA-B | major histocompatibility complex_class | 5.20569 | 2.38009 | 1.09E−26 |
| | I_ B | | | |
| KCNN3 | potassium channel_ calcium activated | 5.20126 | 2.37886 | 0.000383 |
| | intermediate/small conductance | | | |
| | subfamily N alpha_ member 3 | | | |
| CRLF1 | cytokine receptor-like factor 1 | 5.15671 | 2.36645 | 1.13E−11 |
| ATP8A1 | ATPase_ aminophospholipid transporter | 5.15531 | 2.36606 | 1.96E−07 |
| | (APLT)_ class I_ type 8A_ member 1 | | | |
| TRIM9 | tripartite motif containing 9 | 5.15374 | 2.36562 | 0.000187 |
| KCNA7 | potassium channel_ voltage gated shaker | 5.15228 | 2.36521 | 2.72E−05 |
| | related subfamily A_ member 7 | | | |
| TAGLN3 | transgelin 3 | 5.14806 | 2.36403 | 4.16E−08 |
| PRKCG | protein kinase C_ gamma | 5.13449 | 2.36022 | 1.20E−05 |
| SPON1 | spondin 1_ extracellular matrix protein | 5.12098 | 2.35642 | 5.19E−09 |
| PKD1L2 | polycystic kidney disease 1-like 2 | 5.12063 | 2.35632 | 0.000127 |
| | (gene/pseudogene) | | | |
| PKNOX2 | PBX/knotted 1 homeobox 2 | 5.11513 | 2.35477 | 0.000112 |
| LOC100129203 | uncharacterized LOC100129203 | 5.10379 | 2.35157 | 0.001332 |
| DOK6 | docking protein 6 | 5.07399 | 2.34312 | 4.34E−05 |
| TNFSF4 | tumor necrosis factor (ligand) | 5.05892 | 2.33883 | 1.26E−10 |
| | superfamily_ member 4 | | | |
| CHDH | choline dehydrogenase | 5.05647 | 2.33813 | 0.000408 |
| CAMSAP3 | calmodulin regulated spectrin-associated | 5.04422 | 2.33463 | 0.000936 |
| | protein family_ member 3 | | | |
| NEDD4L | neural precursor cell expressed_ | 5.03796 | 2.33284 | 8.36E−17 |
| | developmentally down-regulated 4-like_ | | | |
| | E3 ubiquitin protein ligase | | | |
| ZNF702P | zinc finger protein 702_ pseudogene | 5.02959 | 2.33044 | 2.26E−06 |
| PPP1R1C | protein phosphatase 1_ regulatory | 5.01083 | 2.32505 | 0.000132 |
| | (inhibitor) subunit 1C | | | |
| CPE | carboxypeptidase E | 5.00021 | 2.32199 | 2.22E−05 |

TABLE 6
Genes more highly expressed in UCB-MSCs compared to HMCs

| | | | Log | |
|---|---|---|---|---|
| | | Fold | Fold | |
| Name | Description | Change | Change | p-Value |
| MEG3 | maternally expressed 3 (non-protein | −17630.7 | −14.1058 | 7.58E−194 |
| | coding) | | | |
| CAT | catalase | −1511.12 | −10.5614 | 1.14E−99 |
| DYNLT3 | dynein_ light_ chain_ Tctex-type 3 | −1417.76 | −10.4694 | 5.79E−88 |
| ALDH1A1 | aldehyde dehydrogenase 1 family_{—} | −1170.82 | −10.1933 | 5.05E−179 |
| | member A1 | | | |
| S100A6 | S100 calcium binding protein A6 | −895.544 | −9.80662 | 5.29E−222 |
| GSTT1 | glutathione S-transferase theta 1 | −681.793 | −9.41319 | 2.12E−66 |
| CTSF | cathepsin F | −302.374 | −8.24019 | 7.74E−45 |
| CMKLR1 | chemerin chemokine-like receptor 1 | −284.33 | −8.15142 | 2.15E−43 |
| FLG-AS1 | FLG antisense RNA 1 | −246.966 | −7.94817 | 1.08E−41 |
| KRBOX1 | KRAB box domain containing 1 | −229.441 | −7.84198 | 4.89E−41 |
| LYNX1 | Ly6/neurotoxin 1 | −199.238 | −7.63835 | 7.06E−36 |
| FMOD | fibromodulin | −191.276 | −7.57951 | 1.15E−56 |
| ZNF662 | zinc finger protein 662 | −174.724 | −7.44893 | 1.18E−40 |
| LMO3 | LIM domain only 3 (rhombotin-like 2) | −170.074 | −7.41002 | 8.31E−49 |
| CNTN3 | contactin 3 (plasmacytoma associated) | −162.07 | −7.34047 | 1.38E−64 |
| CXCL1 | chemokine (C-X-C motif) ligand 1 | −149.21 | −7.2212 | 2.41E−66 |
| | (melanoma growth stimulating | | | |
| | activity_ alpha) | | | |
| IRX3 | iroquois homeobox 3 | −148.228 | −7.21167 | 1.60E−65 |
| LINC01133 | long intergenic non-protein coding | −143.929 | −7.16921 | 8.35E−65 |
| | RNA 1133 | | | |
| CCDC36 | coiled-coil domain containing 36 | −142.12 | −7.15097 | 7.50E−32 |
| LOC400043 | uncharacterized LOC400043 | −135.246 | −7.07944 | 2.84E−43 |
| CXCL6 | chemokine (C-X-C motif) ligand 6 | −134.778 | −7.07444 | 4.76E−46 |
| LOC101929369 | NA | −133.058 | −7.05591 | 2.42E−39 |
| C5orf63 | chromosome 5 open reading frame 63 | −130.609 | −7.02911 | 1.23E−35 |
| ANGPTL1 | angiopoietin-like 1 | −129.967 | −7.022 | 1.55E−47 |
| MEG8 | maternally expressed 8 (non-protein | −128.676 | −7.0076 | 1.67E−30 |
| | coding) | | | |
| BHMT2 | betaine--homocysteine S- | −114.938 | −6.84471 | 8.37E−28 |
| | methyltransferase 2 | | | |
| RTN1 | reticulon 1 | −109.648 | −6.77673 | 6.25E−56 |
| FLG | filaggrin | −100.449 | −6.65032 | 8.20E−26 |
| PCDHGA12 | protocadherin gamma subfamily A_ 12 | −99.7593 | −6.64038 | 2.95E−35 |
| PI16 | peptidase inhibitor 16 | −98.0204 | −6.61501 | 2.34E−34 |
| FOXQ1 | forkhead box Q1 | −97.2354 | −6.60341 | 5.15E−25 |
| SLC39A4 | solute carrier family 39 (zinc | −96.9111 | −6.59859 | 2.72E−45 |
| | transporter)_ member 4 | | | |
| HOXC8 | homeobox C8 | −91.8113 | −6.5206 | 3.45E−183 |
| SDR42E1 | short chain dehydrogenase/reductase | −88.1232 | −6.46145 | 5.13E−25 |
| | family 42E_ member 1 | | | |
| ZNF300P1 | zinc finger protein 300 pseudogene 1 | −86.6483 | −6.4371 | 5.64E−28 |
| | (functional) | | | |
| PID1 | phosphotyrosine interaction domain | −86.3803 | −6.43263 | 1.87E−48 |
| | containing 1 | | | |
| ABCA8 | ATP-binding cassette_ sub-family A | −82.6607 | −6.36913 | 5.15E−87 |
| | (ABC1)_ member 8 | | | |
| NAALADL 1 | N-acetylated alpha-linked acidic | −81.0517 | −6.34077 | 5.70E−89 |
| | dipeptidase-like 1 | | | |
| GSTM5 | glutathione S-transferase mu 5 | −78.262 | −6.29024 | 7.38E−25 |
| LOC150381 | NA | −78.0664 | −6.28663 | 2.48E−40 |
| SPESP1 | sperm equatorial segment protein 1 | −75.344 | −6.23542 | 7.37E−22 |
| COX7A1 | cytochrome c oxidase subunit VIIa | −71.77 | −6.16531 | 8.83E−41 |
| | polypeptide 1 (muscle) | | | |
| CHI3L1 | chitinase 3-like 1 (cartilage | −71.6066 | −6.16202 | 2.08E−36 |
| | glycoprotein-39) | | | |
| PLD5 | phospholipase D family_ member 5 | −71.5604 | −6.16109 | 3.57E−22 |
| PAX8-AS1 | PAX8 antisense RNA 1 | −70.7006 | −6.14365 | 6.52E−60 |
| LINC00473 | long intergenic non-protein coding | −65.782 | −6.03962 | 8.00E−37 |
| | RNA 473 | | | |
| TNFAIP6 | tumor necrosis factor_ alpha-induced | −65.3624 | −6.03039 | 6.10E−32 |
| | protein 6 | | | |
| CCDC89 | coiled-coil domain containing 89 | −65.2389 | −6.02766 | 3.85E−33 |
| NKAPL | NFKB activating protein-like | −63.0638 | −5.97874 | 1.55E−20 |
| PTGES | prostaglandin E synthase | −61.3844 | −5.9398 | 7.12E−283 |
| IQGAP2 | IQ motif containing GTPase activating | −61.2161 | −5.93584 | 7.26E−46 |
| | protein 2 | | | |
| HOXC-AS1 | HOXC cluster antisense RNA 1 | −61.0945 | −5.93297 | 3.56E−23 |
| CXCL3 | chemokine (C-X-C motif) ligand 3 | −60.7166 | −5.92402 | 3.01E−21 |
| DNAJA4 | DnaJ (Hsp40) homolog_ subfamily A_{—} | −59.1739 | −5.88689 | 4.13E−60 |
| | member 4 | | | |
| LINC00654 | long intergenic non-protein coding | −54.5633 | −5.76986 | 3.95E−29 |
| | RNA 654 | | | |
| MYH13 | myosin_ heavy chain 13_ skeletal | −53.4523 | −5.74018 | 1.10E−19 |
| | muscle | | | |
| CCDC144B | coiled-coil domain containing 144B | −51.3236 | −5.68155 | 5.35E−18 |
| | (pseudogene) | | | |
| CXCL5 | chemokine (C-X-C motif) ligand 5 | −51.2493 | −5.67946 | 9.87E−32 |
| PCDHGB3 | protocadherin gamma subfamily B_ 3 | −51.168 | −5.67717 | 4.94E−18 |
| AARD | alanine and arginine rich domain | −50.3978 | −5.65529 | 1.45E−27 |
| | containing protein | | | |
| CARD16 | caspase recruitment domain family_{—} | −50.1007 | −5.64676 | 1.07E−63 |
| | member 16 | | | |
| GAS1 | growth arrest-specific 1 | −49.8734 | −5.6402 | 4.77E−129 |
| LOC100240735 | uncharacterized LOC100240735 | −49.8499 | −5.63952 | 5.09E−18 |
| CSF3 | colony stimulating factor 3 | −49.1343 | −5.61866 | 1.33E−16 |
| | (granulocyte) | | | |
| HOXC10 | homeobox C10 | −48.8217 | −5.60945 | 2.74E−104 |
| CXCL8 | chemokine (C-X-C motif) ligand 8 | −48.64 | −5.60407 | 1.22E−37 |
| NUPR1 | nuclear protein_ transcriptional | −48.4841 | −5.59944 | 2.59E−81 |
| | regulator_ 1 | | | |
| ZNF572 | zinc finger protein 572 | −48.1552 | −5.58962 | 1.69E−17 |
| HSPB2 | heat shock 27kDa protein 2 | −47.9112 | −5.58229 | 1.64E−17 |
| HOXD8 | homeobox D8 | −47.1374 | −5.5588 | 8.90E−63 |
| GBP4 | guanylate binding protein 4 | −45.9041 | −5.52055 | 4.74E−42 |
| LRRK2 | leucine-rich repeat kinase 2 | −45.7497 | −5.51569 | 6.91E−16 |
| FAM66B | family with sequence similarity 66_{—} | −44.8352 | −5.48656 | 1.09E−16 |
| | member B | | | |
| ISLR | immunoglobulin superfamily | −44.685 | −5.48172 | 1.83E−95 |
| | containing leucine-rich repeat | | | |
| PCDHGA3 | protocadherin gamma subfamily A_ 3 | −44.2636 | −5.46805 | 1.12E−16 |
| ZNF736 | zinc finger protein 736 | −43.1447 | −5.43111 | 3.45E−16 |
| HSD17B7P2 | hydroxysteroid (17-beta) | −42.9582 | −5.42486 | 2.52E−30 |
| | dehydrogenase 7 pseudogene 2 | | | |
| LRRTM3 | leucine rich repeat transmembrane | −42.6848 | −5.41565 | 4.82E−30 |
| | neuronal 3 | | | |
| HGF | hepatocyte growth factor (hepapoietin | −42.505 | −5.40956 | 8.03E−36 |
| | A; scatter factor) | | | |
| ADAMTS4 | ADAM metallopeptidase with | −42.3714 | −5.40502 | 9.49E−138 |
| | thrombospondin type 1 motif_ 4 | | | |
| ZNF257 | zinc finger protein 257 | −41.6912 | −5.38167 | 4.49E−16 |
| GRID2 | glutamate receptor_ ionotropic_ delta 2 | −41.5991 | −5.37848 | 1.54E−18 |
| FGF7 | fibroblast growth factor 7 | −41.044 | −5.3591 | 1.97E−25 |
| PRSS30P | protease_ serine_ 30_ pseudogene | −40.9168 | −5.35462 | 1.00E−15 |
| LINC00506 | long intergenic non-protein coding | −40.2244 | −5.33 | 1.00E−15 |
| | RNA 506 | | | |
| HOXC5 | homeobox C5 | −37.8836 | −5.2435 | 2.08E−41 |
| ADIRF | adipogenesis regulatory factor | −37.6375 | −5.2341 | 1.34E−31 |
| ZFP3 | ZFP3 zinc finger protein | −37.3196 | −5.22186 | 1.35E−56 |
| HYDIN | HYDIN_ axonemal central pair | −37.0798 | −5.21256 | 8.40E−18 |
| | apparatus protein | | | |
| TDO2 | tryptophan 2_3-dioxygenase | −36.9156 | −5.20616 | 2.68E−25 |
| CD200 | CD200 molecule | −36.3481 | −5.18381 | 0.00E+00 |
| HOXC4 | homeobox C4 | −36.3086 | −5.18224 | 1.34E−75 |
| ANXA10 | annexin A10 | −35.674 | −5.1568 | 2.34E−121 |
| LOC284757 | NA | −35.3454 | −5.14345 | 3.92E−16 |
| ZNF311 | zinc finger protein 311 | −34.811 | −5.12147 | 4.19E−40 |
| CASP1 | caspase 1_ apoptosis-related cysteine | −34.3279 | −5.10131 | 1.32E−90 |
| | peptidase | | | |
| C1QTNF7 | C1q and tumor necrosis factor related | −33.2599 | −5.05571 | 1.08E−13 |
| | protein 7 | | | |
| SNORD114-10 | small nucleolar RNA_ C/D box 114-10 | −32.3322 | −5.0149 | 1.14E−13 |
| PCDHGA11 | protocadherin gamma subfamily A_ 11 | −32.1396 | −5.00628 | 9.50E−53 |
| HOXD-AS2 | HOXD cluster antisense RNA 2 | −31.947 | −4.99761 | 3.44E−26 |
| PITX1 | paired-like homeodomain 1 | −31.6427 | −4.9838 | 1.10E−119 |
| ZNF492 | zinc finger protein 492 | −31.3026 | −4.96821 | 1.68E−14 |
| HOXC6 | homeobox C6 | −31.295 | −4.96786 | 4.21E−45 |
| HOXC9 | homeobox C9 | −31.2237 | −4.96457 | 1.02E−30 |
| KCNJ13 | potassium channel_ inwardly | −30.8716 | −4.94821 | 4.29E−17 |
| | rectifying subfamily J_ member 13 | | | |
| IL1B | interleukin 1_ beta | −29.6829 | −4.89156 | 1.93E−46 |
| C11orf86 | chromosome 11 open reading frame 86 | −29.6418 | −4.88956 | 1.78E−20 |
| CSGALNACT1 | chondroitin sulfate N- | −29.5941 | −4.88724 | 2.37E−57 |
| | acetylgalactosaminyltransferase 1 | | | |
| FPR1 | formyl peptide receptor 1 | −29.0064 | −4.8583 | 1.23E−12 |
| LOC728819 | NA | −28.0926 | −4.81212 | 2.57E−12 |
| MLC1 | megalencephalic leukoencephalopathy | −28.0634 | −4.81062 | 4.15E−21 |
| | with subcortical cysts 1 | | | |
| CXCL2 | chemokine (C-X-C motif) ligand 2 | −27.977 | −4.80617 | 4.71E−24 |
| CEACAM22P | carcinoembryonic antigen-related cell | −27.7945 | −4.79673 | 4.37E−12 |
| | adhesion molecule 22_ pseudogene | | | |
| ZNF454 | zinc finger protein 454 | −27.2796 | −4.76975 | 2.00E−15 |
| TDRD9 | tudor domain containing 9 | −26.7334 | −4.74057 | 4.26E−12 |
| FAM198A | family with sequence similarity 198_{—} | −26.5826 | −4.73241 | 8.07E−12 |
| | member A | | | |
| IL21-AS1 | IL21 antisense RNA 1 | −26.2061 | −4.71183 | 9.27E−12 |
| LINC00478 | mir-99a-let-7c cluster host gene | −25.7904 | −4.68876 | 1.29E−17 |
| ZNF439 | zinc finger protein 439 | −25.5938 | −4.67772 | 2.02E−40 |
| KLHDC7B | kelch domain containing 7B | −25.3659 | −4.66482 | 2.63E−32 |
| EN1 | engrailed homeobox 1 | −25.0474 | −4.64659 | 1.53E−10 |
| SLC22A15 | solute carrier family 22_ member 15 | −24.8777 | −4.63678 | 1.45E−62 |
| LOC283683 | uncharacterized LOC283683 | −24.4078 | −4.60927 | 2.99E−14 |
| DOK5 | docking protein 5 | −24.0441 | −4.58761 | 9.96E−25 |
| LINC00922 | long intergenic non-protein coding | −23.9592 | −4.58251 | 4.59E−11 |
| | RNA 922 | | | |
| LINC00865 | long intergenic non-protein coding | −23.941 | −4.58141 | 3.99E−11 |
| | RNA 865 | | | |
| PF4V1 | platelet factor 4 variant 1 | −23.5142 | −4.55546 | 3.02E−10 |
| MLKL | mixed lineage kinase domain-like | −23.4787 | −4.55328 | 3.66E−225 |
| HOXC-AS2 | HOXC cluster antisense RNA 2 | −23.2705 | −4.54043 | 1.46E−23 |
| STAB1 | stabilin 1 | −23.2547 | −4.53945 | 1.35E−19 |
| PTGFR | prostaglandin F receptor (FP) | −23.1644 | −4.53384 | 1.80E−46 |
| HDC | histidine decarboxylase | −23.0207 | −4.52486 | 1.88E−10 |
| IFI44 | interferon-induced protein 44 | −22.9589 | −4.52098 | 1.56E−41 |
| LINC00578 | long intergenic non-protein coding | −22.9554 | −4.52076 | 1.06E−12 |
| | RNA 578 | | | |
| CSTA | cystatin A (stefin A) | −22.5737 | −4.49657 | 1.31E−16 |
| GPNMB | glycoprotein (transmembrane) nmb | −22.3879 | −4.48465 | 4.97E−49 |
| OR51E2 | olfactory receptor_ family 51_{—} | −22.3769 | −4.48394 | 6.76E−26 |
| | subfamily E_ member 2 | | | |
| LINC00856 | long intergenic non-protein coding | −22.3207 | −4.48031 | 2.48E−10 |
| | RNA 856 | | | |
| CFB | complement factor B | −21.9633 | −4.45702 | 8.87E−137 |
| POMC | proopiomelanocortin | −21.6878 | −4.43881 | 3.18E−11 |
| LOC101927468 | uncharacterized LOC101927468 | −21.205 | −4.40633 | 3.00E−10 |
| CD7 | CD7 molecule | −21.1331 | −4.40143 | 4.15E−27 |
| BMPER | BMP binding endothelial regulator | −20.9432 | −4.38841 | 4.46E−52 |
| GSDMA | gasdermin A | −20.921 | −4.38688 | 1.37E−11 |
| SUSD3 | sushi domain containing 3 | −20.5652 | −4.36213 | 1.29E−21 |
| IL1A | interleukin 1_ alpha | −20.5136 | −4.35851 | 3.30E−31 |
| ELOVL3 | ELOVL fatty acid elongase 3 | −20.3658 | −4.34808 | 3.34E−18 |
| PCDHGA6 | protocadherin gamma subfamily A_ 6 | −20.2761 | −4.34171 | 6.62E−25 |
| IGJ | joining chain of multimeric IgA and | −19.8671 | −4.31231 | 3.70E−12 |
| | IgM | | | |
| SEPSECS-AS1 | SEPSECS antisense RNA 1 (head to | −19.8668 | −4.31229 | 1.20E−09 |
| | head) | | | |
| PPP4R4 | protein phosphatase 4_ regulatory | −19.4997 | −4.28538 | 3.86E−40 |
| | subunit 4 | | | |
| CCL20 | chemokine (C-C motif) ligand 20 | −19.484 | −4.28422 | 1.44E−14 |
| DEPTOR | DEP domain containing MTOR- | −19.4709 | −4.28325 | 9.47E−33 |
| | interacting protein | | | |
| ANKRD7 | ankyrin repeat domain 7 | −19.4419 | −4.2811 | 1.58E−09 |
| C3 | complement component 3 | −19.2979 | −4.27037 | 2.08E−21 |
| APOL1 | apolipoprotein L_ 1 | −19.0488 | −4.25163 | 3.81E−37 |
| ITGBL1 | integrin_ beta-like 1 (with EGF-like | −18.9388 | −4.24327 | 1.78E−180 |
| | repeat domains) | | | |
| PCDHGA4 | protocadherin gamma subfamily A_ 4 | −18.6151 | −4.2184 | 1.07E−14 |
| SLC19A3 | solute carrier family 19 (thiamine | −18.5448 | −4.21294 | 5.33E−16 |
| | transporter)_ member 3 | | | |
| CCL5 | chemokine (C-C motif) ligand 5 | −18.4312 | −4.20408 | 2.95E−11 |
| MIR656 | microRNA 656 | −18.4149 | −4.2028 | 3.88E−09 |
| MYH1 | myosin_ heavy chain 1_ skeletal | −18.0365 | −4.17285 | 5.52E−09 |
| | muscle_ adult | | | |
| PDPN | podoplanin | −17.8868 | −4.16082 | 2.51E−17 |
| ZNF560 | zinc finger protein 560 | −17.6976 | −4.14548 | 6.02E−17 |
| HRNR | hornerin | −17.6899 | −4.14485 | 1.10E−08 |
| CNKSR2 | connector enhancer of kinase | −17.623 | −4.13939 | 1.51E−09 |
| | suppressor of Ras 2 | | | |
| C21orf119 | URB1 antisense RNA 1 (head to head) | −17.6194 | −4.13909 | 2.07E−45 |
| SNORD114-1 | small nucleolar RNA_ C/D box 114-1 | −17.5184 | −4.1308 | 6.92E−09 |
| PCDHGA7 | protocadherin gamma subfamily A_7 | −17.5156 | −4.13057 | 2.41E−19 |
| SLC22A3 | solute carrier family 22 (organic cation | −17.5078 | −4.12993 | 1.29E−63 |
| | transporter)_ member 3 | | | |
| LOC100507540 | NA | −17.3144 | −4.1139 | 3.58E−11 |
| ZNF595 | zinc finger protein 595 | −17.2473 | −4.1083 | 3.38E−37 |
| LOC100506834 | uncharacterized LOC100506834 | −17.2068 | −4.10491 | 2.84E−25 |
| DDX43 | DEAD (Asp-Glu-Ala-Asp) box | −17.0631 | −4.09281 | 1.50E−09 |
| | polypeptide 43 | | | |
| SAMD9L | sterile alpha motif domain containing | −16.9705 | −4.08496 | 9.90E−44 |
| | 9-like | | | |
| ZNF578 | zinc finger protein 578 | −16.7751 | −4.06825 | 6.41E−26 |
| FAM20A | family with sequence similarity 20_{—} | −16.5262 | −4.04668 | 3.43E−12 |
| | member A | | | |
| ALDH1A3 | aldehyde dehydrogenase 1 family_{—} | −16.4835 | −4.04295 | 1.93E−28 |
| | member A3 | | | |
| LINC00839 | long intergenic non-protein coding | −16.3055 | −4.02729 | 6.19E−08 |
| | RNA 839 | | | |
| LOC101926935 | uncharacterized LOC101926935 | −16.0385 | −4.00347 | 2.44E−08 |
| HSPA2 | heat shock 70kDa protein 2 | −15.5047 | −3.95463 | 1.15E−64 |
| SGCD | sarcoglycan_ delta (35kDa dystrophin- | −15.4663 | −3.95106 | 8.53E−25 |
| | associated glycoprotein) | | | |
| AKR1C2 | aldo-keto reductase family 1_ member | −15.1928 | −3.92532 | 5.86E−18 |
| | C2 | | | |
| FAM106A | family with sequence similarity 106_{—} | −15.175 | −3.92362 | 6.49E−08 |
| | member A | | | |
| SPON2 | spondin 2_ extracellular matrix protein | −15.051 | −3.91179 | 5.07E−25 |
| CNTNAP2 | contactin associated protein-like 2 | −14.8446 | −3.89187 | 1.63E−17 |
| BRINP3 | bone morphogenetic protein/retinoic | −14.776 | −3.88518 | 3.70E−10 |
| | acid inducible neural-specific 3 | | | |
| ZNF280A | zinc finger protein 280A | −14.7238 | −3.88008 | 1.26E−07 |
| SYT11 | synaptotagmin XI | −14.71 | −3.87873 | 2.65E−18 |
| IL13RA2 | interleukin 13 receptor_ alpha 2 | −14.6618 | −3.87399 | 7.77E−08 |
| HTR2A | 5-hydroxytryptamine (serotonin) | −14.6491 | −3.87274 | 9.16E−08 |
| | receptor 2A_ G protein-coupled | | | |
| FDCSP | follicular dendritic cell secreted protein | −14.606 | −3.86849 | 1.59E−07 |
| PF4 | platelet factor 4 | −14.5247 | −3.86044 | 1.20E−07 |
| LRRN4CL | LRRN4 C-terminal like | −14.4001 | −3.84801 | 1.50E−36 |
| COMT | catechol-O-methyltransferase | −14.3741 | −3.8454 | 4.90E−145 |
| HOXA1 | homeobox A1 | −14.3096 | −3.83891 | 5.13E−52 |
| PDE7B | phosphodiesterase 7B | −14.2867 | −3.8366 | 4.03E−98 |
| RAD21-AS1 | RAD21 antisense RNA 1 | −14.2782 | −3.83574 | 9.85E−11 |
| CBLC | Cbl proto-oncogene C_ E3 ubiquitin | −14.2299 | −3.83085 | 1.46E−08 |
| | protein ligase | | | |
| PRSS3 | protease_ serine_ 3 | −14.22 | −3.82985 | 2.89E−19 |
| STXBP5L | syntaxin binding protein 5-like | −14.064 | −3.81393 | 3.06E−22 |
| AMPH | amphiphysin | −14.0368 | −3.81114 | 9.92E−48 |
| FAM50B | family with sequence similarity 50_{—} | −13.9849 | −3.8058 | 9.58E−38 |
| | member B | | | |
| MYH8 | myosin_ heavy chain 8_ skeletal | −13.9336 | −3.8005 | 3.09E−08 |
| | muscle_ perinatal | | | |
| PRPH2 | peripherin 2 (retinal degeneration_{—} | −13.832 | −3.78994 | 3.29E−13 |
| | slow) | | | |
| ARHGAP20 | Rho GTPase activating protein 20 | −13.8103 | −3.78767 | 5.09E−20 |
| SPOCK3 | sparc/osteonectin_ cwcv and kazal-like | −13.79 | −3.78555 | 1.19E−13 |
| | domains proteoglycan (testican) 3 | | | |
| HOXA10-AS | HOXA10 antisense RNA | −13.646 | −3.77041 | 1.07E−23 |
| GREM2 | gremlin 2_ DAN family BMP | −13.637 | −3.76945 | 7.24E−18 |
| | antagonist | | | |
| C11orf70 | chromosome 11 open reading frame 70 | −13.5827 | −3.7637 | 1.23E−56 |
| PCDHGA8 | protocadherin gamma subfamily A_8 | −13.484 | −3.75318 | 2.54E−63 |
| SLC9A9 | solute carrier family 9_ subfamily A | −13.3834 | −3.74237 | 2.92E−41 |
| | (NHE9_ cation proton antiporter 9)_{—} | | | |
| | member 9 | | | |
| ZNF528 | zinc finger protein 528 | −13.3712 | −3.74106 | 3.29E−35 |
| HOXC11 | homeobox C11 | −13.3183 | −3.73534 | 5.42E−20 |
| HOTAIR | HOX transcript antisense RNA | −13.2446 | −3.72733 | 1.66E−23 |
| HOXA9 | homeobox A9 | −12.9587 | −3.69585 | 2.40E−46 |
| GALNT12 | polypeptide N- | −12.9507 | −3.69496 | 6.77E−23 |
| | acetylgalactosaminyltransferase 12 | | | |
| PDE2A | phosphodiesterase 2A_ cGMP- | −12.9165 | −3.69114 | 2.56E−12 |
| | stimulated | | | |
| PRSS12 | protease_ serine_ 12 (neurotrypsin_{—} | −12.9162 | −3.69111 | 2.60E−84 |
| | motopsin) | | | |
| LINC00707 | long intergenic non-protein coding | −12.9156 | −3.69104 | 9.57E−21 |
| | RNA 707 | | | |
| CHRDL2 | chordin-like 2 | −12.9061 | −3.68998 | 8.82E−08 |
| PCDHGA5 | protocadherin gamma subfamily A_ 5 | −12.7366 | −3.67091 | 3.66E−17 |
| PPAPDC3 | phosphatidic acid phosphatase type 2 | −12.6247 | −3.65818 | 4.50E−56 |
| | domain containing 3 | | | |
| ST6GALNAC5 | ST6 (alpha-N-acetyl-neuraminy1-2_3- | −12.604 | −3.65581 | 1.09E−65 |
| | beta-galactosy1-1_3)-N- | | | |
| | acetylgalactosaminide alpha-2_6- | | | |
| | sialyltransferase 5 | | | |
| LIN7A | lin-7 homolog A (C. elegans) | −12.5604 | −3.65081 | 2.64E−53 |
| PTPRQ | protein tyrosine phosphatase_ receptor | −12.554 | −3.65008 | 5.16E−36 |
| | type_ Q | | | |
| FAM27A | family with sequence similarity 27_{—} | −12.3804 | −3.62999 | 9.74E−07 |
| | member C | | | |
| CNTN5 | contactin 5 | −12.3042 | −3.62108 | 8.87E−13 |
| IL1RN | interleukin 1 receptor antagonist | −12.2859 | −3.61893 | 9.35E−07 |
| HS6ST3 | heparan sulfate 6-O-sulfotransferase 3 | −12.2168 | −3.61079 | 9.07E−07 |
| SNORD113-4 | small nucleolar RNA_ C/D box 113-4 | −12.116 | −3.59884 | 9.47E−07 |
| PRSS21 | protease_ serine_ 21 (testisin) | −12.0628 | −3.59249 | 1.80E−06 |
| TEKT4P2 | tektin 4 pseudogene 2 | −12.0135 | −3.58658 | 2.77E−06 |
| SYBU | syntabulin (syntaxin-interacting) | −11.9685 | −3.58117 | 1.77E−17 |
| P2RX1 | purinergic receptor P2X_ ligand gated | −11.86 | −3.56803 | 1.82E−23 |
| | ion channel_ 1 | | | |
| IRX5 | iroquois homeobox 5 | −11.7574 | −3.5555 | 1.53E−33 |
| ENTPD1-AS1 | ENTPD1 antisense RNA 1 | −11.7407 | −3.55345 | 1.43E−06 |
| RBM47 | RNA binding motif protein 47 | −11.7375 | −3.55305 | 6.27E−62 |
| RFX8 | RFX family member 8_ lacking RFX | −11.7343 | −3.55266 | 5.37E−18 |
| | DNA binding domain | | | |
| DHX58 | DEXH (Asp-Glu-X-His) box | −11.6685 | −3.54455 | 1.82E−36 |
| | polypeptide 58 | | | |
| ESPNL | espin-like | −11.6038 | −3.53652 | 1.55E−21 |
| SPATA41 | spermatogenesis associated 41 (non- | −11.5983 | −3.53584 | 2.01E−07 |
| | protein coding) | | | |
| SH3GL3 | SH3-domain GRB2-like 3 | −11.5783 | −3.53335 | 5.54E−17 |
| NDUFA4L2 | NADH dehydrogenase (ubiquinone) 1 | −11.5191 | −3.52595 | 3.08E−25 |
| | alpha subcomplex_ 4-like 2 | | | |
| GGT5 | gamma-glutamyltransferase 5 | −11.506 | −3.52432 | 1.42E−80 |
| ASS1 | argininosuccinate synthase 1 | −11.4005 | −3.51103 | 4.39E−41 |
| LOC645638 | NA | −11.3763 | −3.50796 | 8.68E−27 |
| SLPI | secretory leukocyte peptidase inhibitor | −11.303 | −3.49863 | 2.64E−06 |
| AGBL2 | ATP/GTP binding protein-like 2 | −11.295 | −3.49761 | 3.66E−16 |
| HOXD4 | homeobox D4 | −11.2388 | −3.49041 | 1.49E−21 |
| TMEM155 | transmembrane protein 155 | −11.2145 | −3.48729 | 1.59E−14 |
| SMIM21 | small integral membrane protein 21 | −11.171 | −3.48168 | 2.91E−06 |
| C9orf170 | chromosome 9 open reading frame 170 | −11.1377 | −3.47738 | 4.33E−07 |
| ECM2 | extracellular matrix protein 2_ female | −11.0978 | −3.4722 | 1.52E−29 |
| | organ and adipocyte specific | | | |
| CHRNA9 | cholinergic receptor_ nicotinic_ alpha | −11.0708 | −3.46869 | 6.89E−07 |
| | 9 (neuronal) | | | |
| PCDHGA2 | protocadherin gamma subfamily A_ 2 | −11.0172 | −3.46168 | 8.04E−11 |
| NAT2 | N-acetyltransferase 2 (arylamine N- | −10.9863 | −3.45764 | 5.16E−10 |
| | acetyltransferase) | | | |
| EMX2 | empty spiracles homeobox 2 | −10.9733 | −3.45593 | 3.76E−07 |
| PDIA2 | protein disulfide isomerase family A_{—} | −10.9385 | −3.45134 | 6.66E−10 |
| | member 2 | | | |
| OGN | osteoglycin | −10.9193 | −3.44881 | 1.20E−07 |
| LTF | lactotransferrin | −10.8719 | −3.44253 | 4.86E−06 |
| GRM6 | glutamate receptor_ metabotropic 6 | −10.8433 | −3.43873 | 1.06E−06 |
| PRR34 | proline rich 34 | −10.8229 | −3.43601 | 9.05E−09 |
| USP32P2 | ubiquitin specific peptidase 32 | −10.7465 | −3.42579 | 1.18E−07 |
| | pseudogene 2 | | | |
| GPAT2 | glycerol-3-phosphate acyltransferase | −10.6944 | −3.41879 | 1.94E−10 |
| | 2_ mitochondrial | | | |
| PTGS2 | prostaglandin-endoperoxide synthase 2 | −10.678 | −3.41657 | 4.22E−30 |
| | (prostaglandin G/H synthase and | | | |
| | cyclooxygenase) | | | |
| C9orf64 | chromosome 9 open reading frame 64 | −10.6157 | −3.40813 | 5.46E−28 |
| LINC00884 | long intergenic non-protein coding | −10.5556 | −3.39994 | 8.61E−07 |
| | RNA 884 | | | |
| GUCY1B3 | guanylate cyclase 1_ soluble_ beta 3 | −10.5296 | −3.39638 | 6.23E−36 |
| DMRT2 | doublesex and mab-3 related | −10.4519 | −3.3857 | 9.81E−06 |
| | transcription factor 2 | | | |
| GBP5 | guanylate binding protein 5 | −10.4326 | −3.38302 | 4.00E−07 |
| MYOT | myotilin | −10.3531 | −3.37199 | 4.38E−10 |
| PCDHGB1 | protocadherin gamma subfamily B_ 1 | −10.3161 | −3.36682 | 8.50E−10 |
| EPGN | epithelial mitogen | −10.2797 | −3.36172 | 3.50E−09 |
| MME | membrane metallo-endopeptidase | −10.2755 | −3.36114 | 5.88E−25 |
| ST3GAL6- | ST3GAL6 antisense RNA 1 | −10.269 | −3.36023 | 1.58E−12 |
| AS1 | | | | |
| ATP8B4 | ATPase_ class I_ type 8B_ member 4 | −10.242 | −3.35643 | 9.62E−16 |
| TSTD1 | thiosulfate sulfurtransferase | −10.231 | −3.35488 | 2.07E−13 |
| | (rhodanese)-like domain containing 1 | | | |
| LOC102724927 | uncharacterized LOC102724927 | −10.1447 | −3.34265 | 1.27E−18 |
| LIPC | lipase_ hepatic | −10.1202 | −3.33917 | 9.64E−06 |
| RAETIE | retinoic acid early transcript 1E | −10.1052 | −3.33702 | 4.21E−07 |
| EMX2OS | EMX2 opposite strand/antisense RNA | −10.0321 | −3.32655 | 1.01E−05 |
| LINC00540 | long intergenic non-protein coding | −9.99412 | −3.32108 | 1.10E−05 |
| | RNA 540 | | | |
| RSPO2 | R-spondin 2 | −9.98734 | −3.3201 | 9.05E−06 |
| PDE4B | phosphodiesterase 4B_ cAMP-specific | −9.96763 | −3.31725 | 4.87E−44 |
| LCNL1 | lipocalin-like 1 | −9.96645 | −3.31708 | 4.54E−26 |
| HOTAIRM1 | HOXA transcript antisense RNA_{—} | −9.94899 | −3.31455 | 2.93E−11 |
| | myeloid-specific 1 | | | |
| DMGDH | dimethylglycine dehydrogenase | −9.88328 | −3.30499 | 4.90E−09 |
| SIGLEC10 | sialic acid binding Ig-like lectin 10 | −9.87917 | −3.30439 | 1.40E−06 |
| ELANE | elastase_ neutrophil expressed | −9.78058 | −3.28992 | 1.22E−05 |
| CD55 | CD55 molecule_ decay accelerating | −9.66681 | −3.27304 | 1.09E−17 |
| | factor for complement (Cromer blood | | | |
| | group) | | | |
| TNFRSF8 | tumor necrosis factor receptor | −9.66205 | −3.27233 | 3.99E−08 |
| | superfamily_ member 8 | | | |
| MB21D1 | Mab-21 domain containing 1 | −9.5886 | −3.26132 | 2.18E−34 |
| IL1R1 | interleukin 1 receptor type_ I | −9.54616 | −3.25492 | 5.60E−97 |
| SNTG2 | syntrophin_ gamma 2 | −9.4908 | −3.24653 | 5.86E−14 |
| ANPEP | alanyl (membrane) aminopeptidase | −9.46866 | −3.24316 | 1.64E−113 |
| HP09025 | uncharacterized LOC100652929 | −9.45076 | −3.24043 | 1.87E−05 |
| PAX8 | paired box 8 | −9.40977 | −3.23416 | 5.17E−15 |
| IL 1R2 | interleukin 1 receptor_type II | −9.39713 | −3.23222 | 4.46E−07 |
| HOXA10 | homeobox A10 | −9.37189 | −3.22834 | 2.63E−96 |
| ST8SIA4 | ST8 alpha-N-acetyl-neuraminide | −9.36728 | −3.22763 | 1.55E−42 |
| | alpha-2_8-sialyltransferase 4 | | | |
| PIWIL2 | piwi-like RNA-mediated gene | −9.31522 | −3.21959 | 1.57E−10 |
| | silencing 2 | | | |
| COL14A1 | collagen_ type XIV_ alpha 1 | −9.30515 | −3.21803 | 2.33E−09 |
| DHRS3 | dehydrogenase/reductase (SDR family) | −9.29774 | −3.21688 | 2.28E−115 |
| | member 3 | | | |
| MR1 | major histocompatibility complex_{—} | −9.27785 | −3.21379 | 5.21E−24 |
| | class I-related | | | |
| HOXA11 | homeobox A11 | −9.21868 | −3.20456 | 5.28E−303 |
| VTN | vitronectin | −9.19685 | −3.20114 | 6.40E−52 |
| C1QTNF1 | C1q and tumor necrosis factor related | −9.16344 | −3.19589 | 1.36E−28 |
| | protein 1 | | | |
| ABCA6 | ATP-binding cassette_ sub-family A | −9.06131 | −3.17972 | 9.25E−09 |
| | (ABC1)_ member 6 | | | |
| CHRM3 | cholinergic receptor_ muscarinic 3 | −9.03134 | −3.17494 | 2.72E−05 |
| GPR85 | G protein-coupled receptor 85 | −9.02571 | −3.17404 | 7.36E−43 |
| CCL7 | chemokine (C-C motif) ligand 7 | −9.01627 | −3.17253 | 1.82E−13 |
| KLF4 | Kruppel-like factor 4 (gut) | −8.9928 | −3.16877 | 2.83E−34 |
| MALRD1 | MAM and LDL receptor class A | −8.91837 | −3.15678 | 3.50E−05 |
| | domain containing 1 | | | |
| SLC27A2 | solute carrier family 27 (fatty acid | −8.91108 | −3.1556 | 1.64E−10 |
| | transporter)_ member 2 | | | |
| DIRAS3 | DIRAS family_ GTP-binding RAS- | −8.838 | −3.14372 | 9.46E−137 |
| | like 3 | | | |
| CRYAB | crystallin_ alpha B | −8.83359 | −3.143 | 1.22E−23 |
| LINC00968 | long intergenic non-protein coding | −8.82888 | −3.14223 | 2.73E−11 |
| | RNA 968 | | | |
| LAMA2 | laminin_ alpha 2 | −8.81329 | −3.13968 | 1.53E−37 |
| MCTP2 | multiple C2 domains_ transmembrane | −8.79577 | −3.13681 | 2.25E−19 |
| | 2 | | | |
| MIR199A1 | microRNA 199a-1 | −8.77312 | −3.13309 | 2.25E−06 |
| FGF14 | fibroblast growth factor 14 | −8.76655 | −3.13201 | 2.54E−13 |
| PIWIL3 | piwi-like RNA-mediated gene | −8.74695 | −3.12878 | 4.15E−05 |
| | silencing 3 | | | |
| PRG2 | proteoglycan 2_ bone marrow (natural | −8.69683 | −3.12049 | 3.14E−08 |
| | killer cell activator_ eosinophil granule | | | |
| | major basic protein) | | | |
| NMU | neuromedin U | −8.66945 | −3.11594 | 4.96E−05 |
| PTPN20B | protein tyrosine phosphatase_ non- | −8.55964 | −3.09755 | 4.66E−05 |
| | receptor type 20 | | | |
| XAF1 | XIAP associated factor 1 | −8.55869 | −3.09739 | 3.21E−18 |
| ABCC3 | ATP-binding cassette_ sub-family C | −8.55857 | −3.09737 | 3.88E−29 |
| | (CFTR/MRP)_ member 3 | | | |
| HSPA7 | heat shock 70kDa protein 7 (HSP70B) | −8.55436 | −3.09666 | 4.67E−05 |
| SYN3 | synapsin III | −8.54843 | −3.09566 | 4.78E−05 |
| JAKMIP2 | janus kinase and microtubule | −8.53996 | −3.09423 | 2.50E−07 |
| | interacting protein 2 | | | |
| TMCC3 | transmembrane and coiled-coil domain | −8.52719 | −3.09207 | 1.37E−26 |
| | family 3 | | | |
| IL22RA1 | interleukin 22 receptor_ alpha 1 | −8.46823 | −3.08206 | 1.47E−05 |
| ATE1-AS1 | ATE1 antisense RNA 1 | −8.42987 | −3.07551 | 5.34E−05 |
| KCND2 | potassium channel_ voltage gated Shal | −8.41096 | −3.07227 | 1.61E−10 |
| | related subfamily D_ member 2 | | | |
| MIR410 | microRNA 410 | −8.40927 | −3.07198 | 5.23E−05 |
| LINC00664 | long intergenic non-protein coding | −8.4 | −3.07039 | 5.69E−05 |
| | RNA 664 | | | |
| MKRN3 | makorin ring finger protein 3 | −8.37959 | −3.06688 | 6.58E−05 |
| ANKRD2 | ankyrin repeat domain 2 (stretch | −8.36891 | −3.06504 | 2.42E−14 |
| | responsive muscle) | | | |
| COL8A2 | collagen_ type VIII_ alpha 2 | −8.26996 | −3.04788 | 8.49E−24 |
| CFHR1 | complement factor H-related 1 | −8.24809 | −3.04406 | 7.97E−12 |
| TRPV3 | transient receptor potential cation | −8.2018 | −3.03594 | 8.67E−13 |
| | channel_ subfamily V_member 3 | | | |
| GAL3ST1 | galactose-3-O-sulfotransferase 1 | −8.17971 | −3.03205 | 7.89E−05 |
| PCDHGB5 | protocadherin gamma subfamily B_ 5 | −8.17002 | −3.03034 | 3.34E−09 |
| TFPI2 | tissue factor pathway inhibitor 2 | −8.1526 | −3.02726 | 4.98E−24 |
| LPO | lactoperoxidase | −8.10836 | −3.01941 | 1.89E−05 |
| EVI2B | ecotropic viral integration site 2B | −8.07532 | −3.01352 | 5.75E−10 |
| FRMPD1 | FERM and PDZ domain containing 1 | −8.0519 | −3.00933 | 3.56E−05 |
| B4GALNT1 | beta-1_4-N-acetyl-galactosaminyl | −8.04934 | −3.00887 | 1.81E−22 |
| | transferase 1 | | | |
| TRPA1 | transient receptor potential cation | −8.02538 | −3.00457 | 1.88E−06 |
| | channel_ subfamily A_ member 1 | | | |
| ASB2 | ankyrin repeat and SOCS box | −7.95317 | −2.99153 | 5.28E−17 |
| | containing 2 | | | |
| HOXD3 | homeobox D3 | −7.92077 | −2.98564 | 8.03E−07 |
| POM121L9P | POM121 transmembrane nucleoporin- | −7.87866 | −2.97795 | 1.58E−09 |
| | like 9_ pseudogene | | | |
| PSG5 | pregnancy specific beta-1-glycoprotein | −7.82847 | −2.96873 | 1.32E−09 |
| | 5 | | | |
| LOC654342 | lymphocyte-specific protein 1 | −7.8218 | −2.9675 | 3.42E−73 |
| | pseudogene | | | |
| HOXA3 | homeobox A3 | −7.76922 | −2.95777 | 1.04E−15 |
| HOXC-AS3 | HOXC cluster antisense RNA 3 | −7.75002 | −2.9542 | 6.68E−09 |
| CDSN | corneodesmosin | −7.74895 | −2.954 | 7.46E−07 |
| PLEKHA7 | pleckstrin homology domain | −7.71957 | −2.94852 | 1.05E−16 |
| | containing_ family A member 7 | | | |
| GRIK2 | glutamate receptor_ ionotropic_{—} | −7.70925 | −2.94659 | 2.40E−44 |
| | kainate 2 | | | |
| FXYD1 | FXYD domain containing ion | −7.67911 | −2.94094 | 3.54E−13 |
| | transport regulator 1 | | | |
| TRIM4 | tripartite motif containing 4 | −7.65127 | −2.9357 | 3.26E−64 |
| PP12613 | uncharacterized LOC100192379 | −7.61931 | −2.92966 | 3.85E−05 |
| KDR | kinase insert domain receptor | −7.59337 | −2.92474 | 1.08E−31 |
| MIR10B | microRNA 10b | −7.59337 | −2.92474 | 0.000142 |
| TSPAN32 | tetraspanin 32 | −7.56594 | −2.91952 | 7.50E−09 |
| TRPM3 | transient receptor potential cation | −7.55714 | −2.91784 | 3.49E−09 |
| | channel_ subfamily M_ member 3 | | | |
| RGL3 | ral guanine nucleotide dissociation | −7.55567 | −2.91756 | 7.95E−09 |
| | stimulator-like 3 | | | |
| CXCL14 | chemokine (C-X-C motif) ligand 14 | −7.54181 | −2.91491 | 5.44E−10 |
| RBM46 | RNA binding motif protein 46 | −7.53465 | −2.91354 | 0.000159 |
| KCNQ3 | potassium channel_ voltage gated | −7.52817 | −2.9123 | 4.08E−20 |
| | KQT-like subfamily Q_ member 3 | | | |
| FAM225A | family with sequence similarity 225_{—} | −7.51587 | −2.90994 | 0.000168 |
| | member A (non-protein coding) | | | |
| TRIM29 | tripartite motif containing 29 | −7.49381 | −2.9057 | 4.35E−15 |
| PRDM1 | PR domain containing 1_ with ZNF | −7.48966 | −2.9049 | 5.11E−33 |
| | domain | | | |
| HIST2H2BA | histone cluster 2_ H2ba (pseudogene) | −7.48011 | −2.90306 | 1.97E−12 |
| SUSD2 | sushi domain containing 2 | −7.47804 | −2.90266 | 2.34E−06 |
| HPD | 4-hydroxyphenylpyruvate dioxygenase | −7.46064 | −2.8993 | 4.25E−10 |
| GPR115 | adhesion G protein-coupled receptor | −7.46028 | −2.89923 | 2.61E−26 |
| | F4 | | | |
| PHYHIP | phytanoyl-CoA 2-hydroxylase | −7.44742 | −2.89674 | 6.50E−21 |
| | interacting protein | | | |
| SPN | sialophorin | −7.40741 | −2.88897 | 1.04E−09 |
| FAM109B | family with sequence similarity 109_{—} | −7.38363 | −2.88433 | 1.52E−90 |
| | member B | | | |
| LOC101926892 | uncharacterized LOC101926892 | −7.34595 | −2.87695 | 0.000183 |
| CASC1 | cancer susceptibility candidate 1 | −7.33019 | −2.87385 | 4.51E−06 |
| PRND | prion protein 2 (dublet) | −7.32328 | −2.87249 | 0.000189 |
| KCNT2 | potassium channel_ sodium activated | −7.26433 | −2.86083 | 3.16E−13 |
| | subfamily T_ member 2 | | | |
| GBP7 | guanylate binding protein 7 | −7.24964 | −2.85791 | 0.000204 |
| TYMP | thymidine phosphorylase | −7.24643 | −2.85727 | 1.99E−51 |
| P4HA3 | prolyl 4-hydroxylase_ alpha | −7.23127 | −2.85425 | 1.01E−28 |
| | polypeptide III | | | |
| MX2 | MX dynamin-like GTPase 2 | −7.22406 | −2.85281 | 1.96E−43 |
| ATP6V0A4 | ATPase_ H+ transporting_ lysosomal | −7.16531 | −2.84103 | 3.08E−06 |
| | V0 subunit a4 | | | |
| IL6 | interleukin 6 | −7.16268 | −2.8405 | 6.92E−09 |
| SCRG1 | stimulator of chondrogenesis 1 | −7.14171 | −2.83627 | 3.06E−33 |
| ENPP4 | ectonucleotide | −7.11247 | −2.83035 | 2.30E−06 |
| | pyrophosphatase/phosphodiesterase 4 | | | |
| | (putative) | | | |
| GCH1 | GTP cyclohydrolase 1 | −7.09022 | −2.82583 | 2.11E−20 |
| LOC102724224 | NA | −7.07054 | −2.82182 | 5.08E−15 |
| ZNF726 | zinc finger protein 726 | −7.05854 | −2.81937 | 2.09E−07 |
| TNFRSF11B | tumor necrosis factor receptor | −7.05683 | −2.81902 | 6.55E−11 |
| | superfamily_ member 11b | | | |
| ZNF829 | zinc finger protein 829 | −7.04715 | −2.81704 | 2.15E−54 |
| TULP2 | tubby like protein 2 | −7.04485 | −2.81657 | 1.07E−05 |
| LOC101929319 | uncharacterized LOC101929319 | −7.03261 | −2.81406 | 0.000119 |
| HSPB6 | heat shock protein_ alpha-crystallin- | −7.01854 | −2.81117 | 1.15E−21 |
| | related_ B6 | | | |
| LAMA1 | laminin_ alpha 1 | −7.0143 | −2.8103 | 9.37E−45 |
| LUZP2 | leucine zipper protein 2 | −7.00954 | −2.80932 | 7.61E−31 |
| LOC101928161 | uncharacterized LOC101928161 | −7.00255 | −2.80788 | 6.81E−24 |
| CCDC64B | coiled-coil domain containing 64B | −6.98897 | −2.80508 | 0.000297 |
| CRHR2 | corticotropin releasing hormone | −6.98839 | −2.80496 | 1.30E−06 |
| | receptor 2 | | | |
| TIMP3 | TIMP metallopeptidase inhibitor 3 | −6.988 | −2.80488 | 2.42E−17 |
| OASL | 2′-5′-oligoadenylate synthetase-like | −6.98369 | −2.80399 | 0.000202 |
| SGCG | sarcoglycan_ gamma (35kDa | −6.94816 | −2.79663 | 9.02E−14 |
| | dystrophin-associated glycoprotein) | | | |
| GYPE | glycophorin E (MNS blood group) | −6.91309 | −2.78933 | 0.000146 |
| TMEM215 | transmembrane protein 215 | −6.8823 | −2.78289 | 0.00033 |
| RADIL | Ras association and DIL domains | −6.86181 | −2.77859 | 2.72E−30 |
| LRRIQ3 | leucine-rich repeats and IQ motif | −6.8524 | −2.77661 | 1.68E−06 |
| | containing 3 | | | |
| NR5A2 | nuclear receptor subfamily 5_ group | −6.85041 | −2.77619 | 0.00012 |
| | A_ member 2 | | | |
| PABPC4L | poly(A) binding protein_ cytoplasmic | −6.84818 | −2.77572 | 4.78E−29 |
| | 4-like | | | |
| PLSCR4 | phospholipid scramblase 4 | −6.843 | −2.77463 | 9.90E−72 |
| LOC100132891 | NA | −6.83817 | −2.77361 | 1.07E−10 |
| LOC100240734 | uncharacterized LOC100240734 | −6.83201 | −2.77231 | 1.59E−05 |
| PRDM6 | PR domain containing 6 | −6.76711 | −2.75854 | 3.35E−06 |
| DNAJC12 | DnaJ (Hsp40) homolog_ subfamily C_{—} | −6.76134 | −2.75731 | 4.24E−23 |
| | member 12 | | | |
| ADAM33 | ADAM metallopeptidase domain 33 | −6.74519 | −2.75386 | 6.75E−10 |
| ANXA8 | annexin A8 | −6.73118 | −2.75086 | 2.82E−18 |
| ZFYVE28 | zinc finger_ FYVE domain containing | −6.72083 | −2.74864 | 3.30E−17 |
| | 28 | | | |
| RRN3P2 | RRN3 homolog_ RNA polymerase I | −6.70557 | −2.74536 | 3.86E−14 |
| | transcription factor pseudogene 2 | | | |
| LINC00271 | long intergenic non-protein coding | −6.69285 | −2.74262 | 0.000178 |
| | RNA 271 | | | |
| LINC01116 | long intergenic non-protein coding | −6.69169 | −2.74237 | 1.07E−40 |
| | RNA 1116 | | | |
| KCNIP3 | Kv channel interacting protein 3_{—} | −6.68459 | −2.74084 | 4.54E−17 |
| | calsenilin | | | |
| SLC30A3 | solute carrier family 30 (zinc | −6.68237 | −2.74036 | 0.000188 |
| | transporter)_ member 3 | | | |
| KCNE4 | potassium channel_ voltage gated | −6.67672 | −2.73914 | 2.01E−26 |
| | subfamily E regulatory beta subunit 4 | | | |
| LOC101927650 | uncharacterized LOC101927650 | −6.66004 | −2.73553 | 0.000222 |
| MEG9 | maternally expressed 9 (non-protein | −6.64975 | −2.7333 | 1.07E−11 |
| | coding) | | | |
| SPAG17 | sperm associated antigen 17 | −6.63263 | −2.72958 | 2.02E−05 |
| RNF112 | ring finger protein 112 | −6.62601 | −2.72814 | 4.94E−13 |
| BACH2 | BTB and CNC homology 1_ basic | −6.60624 | −2.72383 | 3.20E−09 |
| | leucine zipper transcription factor 2 | | | |
| M1AP | meiosis 1 associated protein | −6.59279 | −2.72089 | 0.000184 |
| HOXA7 | homeobox A7 | −6.5864 | −2.71949 | 7.83E−11 |
| PPP1R14C | protein phosphatase 1_ regulatory | −6.57354 | −2.71667 | 0.000573 |
| | (inhibitor) subunit 14C | | | |
| LINC01081 | long intergenic non-protein coding | −6.53252 | −2.70764 | 0.000487 |
| | RNA 1081 | | | |
| MOCOS | molybdenum cofactor sulfurase | −6.52985 | −2.70705 | 4.40E−12 |
| HOXA4 | homeobox A4 | −6.49257 | −2.69879 | 4.09E−14 |
| ATP2B1 | ATPase_ Ca++ transporting_ plasma | −6.48124 | −2.69627 | 8.06E−35 |
| | membrane 1 | | | |
| ALDH3B1 | aldehyde dehydrogenase 3 family_{—} | −6.47078 | −2.69394 | 9.21E−80 |
| | member B1 | | | |
| NKG7 | natural killer cell granule protein 7 | −6.43206 | −2.68528 | 0.000222 |
| S100A4 | S100 calcium binding protein A4 | −6.42248 | −2.68313 | 3.30E−18 |
| LOC441666 | zinc finger protein 91 pseudogene | −6.41349 | −2.68111 | 0.00069 |
| CRISPLD2 | cysteine-rich secretory protein LCCL | −6.38028 | −2.67362 | 2.78E−11 |
| | domain containing 2 | | | |
| SLC38A5 | solute carrier family 38_ member 5 | −6.37595 | −2.67264 | 7.82E−40 |
| KRT34 | keratin 34_ type I | −6.35852 | −2.66869 | 2.57E−05 |
| APOL6 | apolipoprotein L_ 6 | −6.33252 | −2.66278 | 1.01E−35 |
| DPP10 | dipeptidyl-peptidase 10 (non- | −6.31845 | −2.65957 | 7.29E−06 |
| | functional) | | | |
| KLF15 | Kruppel-like factor 15 | −6.30523 | −2.65655 | 6.85E−06 |
| IL33 | interleukin 33 | −6.28503 | −2.65192 | 1.04E−11 |
| HOXA-AS3 | HOXA cluster antisense RNA 3 | −6.27515 | −2.64965 | 1.92E−19 |
| MIR541 | microRNA 541 | −6.22835 | −2.63885 | 0.000695 |
| ANXA8L1 | annexin A8-like 1 | −6.21916 | −2.63672 | 9.58E−20 |
| STRA6 | stimulated by retinoic acid 6 | −6.21395 | −2.63551 | 3.82E−20 |
| PM20D1 | peptidase M20 domain containing 1 | −6.20921 | −2.63441 | 0.000794 |
| GPR1 | G protein-coupled receptor 1 | −6.19666 | −2.63149 | 2.05E−28 |
| IL21R | interleukin 21 receptor | −6.16645 | −2.62444 | 2.53E−09 |
| LOC284889 | NA | −6.15198 | −2.62105 | 2.12E−09 |
| FLJ45974 | NA | −6.10766 | −2.61062 | 0.000858 |
| ABCA9 | ATP-binding cassette_ sub-family A | −6.09827 | −2.6084 | 8.73E−07 |
| | (ABC1)_ member 9 | | | |
| C12orf56 | chromosome 12 open reading frame 56 | −6.09688 | −2.60807 | 0.000122 |
| AKRIB10 | aldo-keto reductase family 1_ member | −6.09206 | −2.60693 | 1.09E−07 |
| | B10 (aldose reductase) | | | |
| MYBPH | myosin binding protein H | −6.06324 | −2.60009 | 0.000347 |
| HSD11B1 | hydroxysteroid (11-beta) | −6.05896 | −2.59907 | 0.001014 |
| | dehydrogenase 1 | | | |
| LOC391322 | D-dopachrome tautomerase-like | −6.05149 | −2.59729 | 1.82E−07 |
| LIPI | lipase_ member I | −6.03465 | −2.59327 | 0.000958 |
| ICAM4 | intercellular adhesion molecule 4 | −6.03072 | −2.59233 | 1.43E−11 |
| | (Landsteiner-Wiener blood group) | | | |
| RTP4 | receptor (chemosensory) transporter | −5.97637 | −2.57927 | 0.00098 |
| | protein 4 | | | |
| LOC100507642 | uncharacterized LOC100507642 | −5.96305 | −2.57605 | 2.82E−29 |
| C4BPB | complement component 4 binding | −5.95421 | −2.57391 | 5.64E−14 |
| | protein_ beta | | | |
| EVA1C | eva-1 homolog C (C. elegans) | −5.94217 | −2.57099 | 2.18E−23 |
| MIR615 | microRNA 615 | −5.8772 | −2.55513 | 0.001088 |
| ASIC5 | acid sensing (proton gated) ion channel | −5.87676 | −2.55502 | 0.001107 |
| | family member 5 | | | |
| TRIM61 | tripartite motif containing 61 | −5.85513 | −2.5497 | 1.21E−16 |
| OLFML3 | olfactomedin-like 3 | −5.84576 | −2.54739 | 2.66E−32 |
| ALPK 1 | alpha-kinase 1 | −5.8373 | −2.5453 | 4.75E−37 |
| LINC00936 | long intergenic non-protein coding | −5.81396 | −2.53952 | 1.41E−32 |
| | RNA 936 | | | |
| LINC00570 | long intergenic non-protein coding | −5.80852 | −2.53817 | 0.001211 |
| | RNA 570 | | | |
| LOC340515 | NA | −5.80538 | −2.53739 | 0.001211 |
| GALNT18 | polypeptide N- | −5.78501 | −2.53232 | 1.74E−19 |
| | acetylgalactosaminyltransferase 18 | | | |
| HOXA11-AS | HOXA11 antisense RNA | −5.778 | −2.53057 | 4.63E−102 |
| HRCT1 | histidine rich carboxyl terminus 1 | −5.77227 | −2.52914 | 9.88E−32 |
| RASIP1 | Ras interacting protein 1 | −5.75398 | −2.52456 | 8.49E−17 |
| FPR2 | formyl peptide receptor 2 | −5.74597 | −2.52255 | 0.001275 |
| IFI44L | interferon-induced protein 44-like | −5.74306 | −2.52182 | 9.82E−09 |
| CCDC147- | CFAP58 antisense RNA 1 (head to | −5.73952 | −2.52093 | 4.88E−09 |
| AS1 | head) | | | |
| LDHAL6B | lactate dehydrogenase A-like 6B | −5.7328 | −2.51924 | 0.000211 |
| KCTD12 | potassium channel tetramerization | −5.71221 | −2.51405 | 6.98E−12 |
| | domain containing 12 | | | |
| GNG2 | guanine nucleotide binding protein (G | −5.71055 | −2.51363 | 8.51E−23 |
| | protein)_ gamma 2 | | | |
| KLHL33 | kelch-like family member 33 | −5.70418 | −2.51202 | 0.001368 |
| ADAMTS1 | ADAM metallopeptidase with | −5.69596 | −2.50994 | 5.60E−24 |
| | thrombospondin type 1 motif_1 | | | |
| SCIN | scinderin | −5.69008 | −2.50845 | 3.26E−08 |
| INSC | inscuteable homolog (Drosophila) | −5.68555 | −2.5073 | 0.001391 |
| DLGAP1 | discs_ large (Drosophila) homolog- | −5.66922 | −2.50315 | 0.000493 |
| | associated protein 1 | | | |
| ZNF354C | zinc finger protein 354C | −5.66871 | −2.50302 | 2.85E−06 |
| ODAM | odontogenic_ ameloblast asssociated | −5.65223 | −2.49882 | 0.001552 |
| LPXN | leupaxin | −5.65203 | −2.49877 | 1.59E−27 |
| NOV | nephroblastoma overexpressed | −5.63045 | −2.49325 | 3.30E−06 |
| HAND2-AS1 | HAND2 antisense RNA 1 (head to | −5.60856 | −2.48763 | 2.11E−115 |
| | head) | | | |
| BCL2A1 | BCL2-related protein A1 | −5.60848 | −2.48761 | 0.000262 |
| ENPP2 | ectonucleotide | −5.60393 | −2.48644 | 9.74E−09 |
| | pyrophosphatase/phosphodiesterase 2 | | | |
| CKM | creatine kinase_ muscle | −5.5897 | −2.48277 | 0.001639 |
| PTGDR | prostaglandin D2 receptor (DP) | −5.57391 | −2.47869 | 0.001624 |
| SLC7A7 | solute carrier family 7 (amino acid | −5.57314 | −2.47849 | 9.34E−50 |
| | transporter light chain_ y + L system)_{—} | | | |
| | member 7 | | | |
| DAW1 | dynein assembly factor with WDR | −5.55902 | −2.47483 | 1.25E−12 |
| | repeat domains 1 | | | |
| OVCH1-AS1 | OVCH1 antisense RNA 1 | −5.55493 | −2.47377 | 0.001707 |
| LRRTM2 | leucine rich repeat transmembrane | −5.53203 | −2.46781 | 3.28E−08 |
| | neuronal 2 | | | |
| KCNE3 | potassium channel_ voltage gated | −5.51951 | −2.46454 | 1.51E−14 |
| | subfamily E regulatory beta subunit 3 | | | |
| IRAK3 | interleukin-1 receptor-associated | −5.51068 | −2.46223 | 3.63E−10 |
| | kinase 3 | | | |
| OGFRL 1 | opioid growth factor receptor-like 1 | −5.50247 | −2.46008 | 1.39E−99 |
| HOXA6 | homeobox A6 | −5.50015 | −2.45947 | 8.54E−12 |
| C1S | complement component 1_ s | −5.47793 | −2.45363 | 2.03E−35 |
| | subcomponent | | | |
| CYSLTR2 | cysteinyl leukotriene receptor 2 | −5.47489 | −2.45283 | 0.000616 |
| PODNL1 | podocan-like 1 | −5.47322 | −2.45239 | 5.63E−51 |
| RBM11 | RNA binding motif protein 11 | −5.4681 | −2.45104 | 0.001847 |
| NID2 | nidogen 2 (osteonidogen) | −5.45103 | −2.44653 | 3.65E−43 |
| BTBD11 | BTB (POZ) domain containing 11 | −5.44775 | −2.44566 | 4.29E−45 |
| KIF6 | kinesin family member 6 | −5.44126 | −2.44394 | 0.000483 |
| LYPD5 | LY6/PLAUR domain containing 5 | −5.42409 | −2.43938 | 0.001979 |
| GCNT1 | glucosaminyl (N-acetyl) transferase 1_{—} | −5.42224 | −2.43889 | 2.28E−139 |
| | core 2 | | | |
| LOC375196 | uncharacterized LOC375196 | −5.42074 | −2.43849 | 7.01E−06 |
| LOC101928200 | NA | −5.3954 | −2.43173 | 6.53E−14 |
| ADAMTS9 | ADAM metallopeptidase with | −5.39529 | −2.4317 | 1.31E−12 |
| | thrombospondin type 1 motif_ 9 | | | |
| LINC00870 | long intergenic non-protein coding | −5.3923 | −2.4309 | 0.002006 |
| | RNA 870 | | | |
| MIR6730 | microRNA 6730 | −5.39211 | −2.43085 | 0.000806 |
| CP | ceruloplasmin (ferroxidase) | −5.37483 | −2.42622 | 0.001242 |
| SULT1E1 | sulfotransferase family 1E_ estrogen- | −5.35869 | −2.42188 | 8.47E−05 |
| | preferring_ member 1 | | | |
| ROR2 | receptor tyrosine kinase-like orphan | −5.35832 | −2.42178 | 2.45E−10 |
| | receptor 2 | | | |
| MFSD7 | major facilitator superfamily domain | −5.34533 | −2.41828 | 1.30E−20 |
| | containing 7 | | | |
| NECAB2 | N-terminal EF-hand calcium binding | −5.33837 | −2.4164 | 3.15E−09 |
| | protein 2 | | | |
| IP6K3 | inositol hexakisphosphate kinase 3 | −5.33649 | −2.41589 | 3.69E−24 |
| INHBE | inhibin_ beta E | −5.3237 | −2.41243 | 5.12E−18 |
| ALDH1L2 | aldehyde dehydrogenase 1 family_{—} | −5.30804 | −2.40818 | 1.83E−18 |
| | member L2 | | | |
| HOXA2 | homeobox A2 | −5.30253 | −2.40668 | 3.64E−08 |
| RCN3 | reticulocalbin 3_ EF-hand calcium | −5.29984 | −2.40595 | 2.01E−34 |
| | binding domain | | | |
| NOL4 | nucleolar protein 4 | −5.2921 | −2.40384 | 0.001181 |
| ISLR2 | immunoglobulin superfamily | −5.28928 | −2.40307 | 0.002359 |
| | containing leucine-rich repeat 2 | | | |
| DHRS4L1 | dehydrogenase/reductase (SDR family) | −5.27818 | −2.40004 | 7.20E−08 |
| | member 4 like 1 | | | |
| HOXA5 | homeobox A5 | −5.27503 | −2.39918 | 7.07E−11 |
| EHHADH | enoyl-CoA_ hydratase/3-hydroxyacyl | −5.26119 | −2.39539 | 1.38E−16 |
| | CoA dehydrogenase | | | |
| LOC101928891 | uncharacterized LOC101928891 | −5.25839 | −2.39462 | 4.53E−08 |
| MGC27382 | uncharacterized MGC27382 | −5.25791 | −2.39449 | 0.002301 |
| SLC12A8 | solute carrier family 12_ member 8 | −5.24943 | −2.39216 | 1.29E−29 |
| CTHRC1 | collagen triple helix repeat containing | −5.23947 | −2.38942 | 1.59E−40 |
| | 1 | | | |
| SNORD127 | small nucleolar RNA_ C/D box 127 | −5.22474 | −2.38536 | 0.001157 |
| BST1 | bone marrow stromal cell antigen 1 | −5.2242 | −2.38521 | 6.04E−21 |
| APOA1 | apolipoprotein A-I | −5.2238 | −2.3851 | 1.64E−06 |
| LINC01169 | long intergenic non-protein coding | −5.21414 | −2.38243 | 0.001091 |
| | RNA 1169 | | | |
| LINC00163 | long intergenic non-protein coding | −5.21393 | −2.38237 | 0.000343 |
| | RNA 163 | | | |
| FHAD1 | forkhead-associated (FHA) | −5.1735 | −2.37114 | 3.98E−12 |
| | phosphopeptide binding domain 1 | | | |
| PDC | phosducin | −5.16569 | −2.36896 | 0.001012 |
| HMOX1 | heme oxygenase 1 | −5.15739 | −2.36664 | 5.29E−29 |
| FAM27E3 | family with sequence similarity 27_{—} | −5.15367 | −2.3656 | 9.91E−07 |
| | member E3 | | | |
| HAS1 | hyaluronan synthase 1 | −5.14635 | −2.36355 | 9.44E−07 |
| LINC00052 | long intergenic non-protein coding | −5.13691 | −2.3609 | 5.40E−09 |
| | RNA 52 | | | |
| EYA2 | EYA transcriptional coactivator and | −5.12816 | −2.35844 | 0.000837 |
| | phosphatase 2 | | | |
| CABP1 | calcium binding protein 1 | −5.1219 | −2.35668 | 0.001407 |
| PCDHGA1 | protocadherin gamma subfamily A_ 1 | −5.11598 | −2.35501 | 6.54E−05 |
| TXNRD2 | thioredoxin reductase 2 | −5.10008 | −2.35052 | 2.65E−45 |
| USP32P1 | ubiquitin specific peptidase 32 | −5.02757 | −2.32986 | 0.001626 |
| | pseudogene 1 | | | |
| DPP4 | dipeptidyl-peptidase 4 | −5.00486 | −2.32333 | 2.66E−16 |

### Example 7—In Vivo Middle Cerebral Artery Occlusion (MCAO) Stroke Model

The HMCs and HMC-EVs of the presently disclosed subject matter were tested in an in vivo model of middle cerebral artery occlusion (MCAO) stroke.

HMCs were generated from the same bank of frozen hemangioblasts described in Example 1.

For HMC-EVs, early passage (passage 4) HMCs were thawed, washed, counted, and plated in Corning CellBIND flasks at a density of 5,000 cells/cm² in RoosterBio RoosterNourish-MSC-XF media. Cells were grown for 96 hours to a confluence of approximately 70-90% for acclimation to the media and cell expansion. At 96 hours, cells were removed from flasks with TripLE dissociation, live cells were counted, and replated at 5,000 cells/cm² in new flasks and fresh media at passage 5. At this passage media can be collected after 96 hours for EV isolation. Cells can be passaged again up to passage 7 for larger volumes of media collection. After media aas harvested for EV isolation, it was clarified to remove cells and debris with differential, low-speed centrifugation at 300×g for 10 minutes and 2,000×g for 20 minutes followed by 0.2 μm vacuum filtration. EVs were isolated from the clarified media using tangential flow filtration (TFF) on the Repligen KR2i system outfitted with a hollow fiber, 300 kDa pore, mPES membrane filter. The approximately 100 nm pore size of filter removed small impurities and retained the EVs. Combined, the clarification and TFF parameters were such that particles between 100 nm and 200 nm in size were isolated. The media was first concentrated by a factor of approximately 10× before it was diafiltered with DPBS to improve sample purity and remove non-EV associated proteins during the TFF process. The diafiltered media was further concentrated so that the final product was concentrated by a factor of approximately 100x. The resulting isolated and concentrated EVs in DPBS were then ready for downstream analyses and could also be further purified using chromatography techniques.

### In Vivo Effects of HMCs and HMC-EVs on Locomotor Skills

MCAO animal models were generated as described herein. Briefly, one day prior to surgical injury, the Body Swing Test was performed to establish the baseline performance using male Sprague-Dawley rats (300-400 g). For each, the rat was held approximately one inch from the base of its tail. It was then elevated to an inch above a surface of a table. The rat was held in the vertical axis, defined as no more than 100 to either the left or the right side. A swing was recorded whenever the rat moved its head out of the vertical axis to either side. The rat must have returned to the vertical position for the next swing to be counted. Thirty total swings were counted. A normal rat typically has an equal number of swings to either side. Following focal ischemia, the rat tends to swing to the contralateral (left) side. After one day of testing, focal cerebral infarcts were made by permanent occlusion of the proximal right middle cerebral artery (MCA) using a modification of the method of Tamura et al. The rats were anesthetized with 1-3% isoflurane in the mixture of N₂O:O₂ (2:1), and were maintained with 1.5-2% isoflurane in the mixture of N₂O:O₂ (2:1). The temporalis muscle was bisected and reflected through an incision made midway between the eye and the eardrum canal. The proximal MCA was exposed through a subtemporal craniectomy without removing the zygomatic arch and without transecting the facial nerve. The artery was then occluded by microbipolar coagulation from just proximal to the olfactory tract to the inferior cerebral vein. Body temperature was maintained at 37.0±1° C. throughout the entire procedure. Cefazolin (40 mg/kg) was given intraperitoneally (i.p.) before MCAO to prevent infections. Buprenorphine, s.c, (˜0.1 mg/kg Simbadol) was given before the MCAO surgery as analgesia. For Sham conditions, animals underwent the same procedure described above without the middle cerebral artery being coagulated.

Treatments were administered on day 1 and day 7 after the MCAO surgery (24 hours and 7 days+/−10%). For HMC treatments, the cells were stored in liquid nitrogen until the day of use. Cells were thawed in a 37° C. water bath, counted, and diluted in the vehicle, Plasma-Lyte A. For HMC-EV treatments, EV aliquots were stored at −80° C. until the day of use. EVs were thawed on ice and either diluted in the vehicle, DPBS, or used as prepared.

On day 1 and day 7 after the MCAO (24 hours and 7 days+/−10⁰/a), animals were anesthetized with 1-3% isoflurane in the 02 and were maintained with 1.5-2% isoflurane in 02. Jugular vein injections were performed by using a 1 ml syringe with a 25G (3/4″) needle attached, 0.5 ml vehicle or cells were injected into the jugular vein. The injection site was compressed for about 1 minute to ensure there was no bleeding. Local injection were performed by using a 50 microliter Hamilton syringe with a 26G needle attached, 10 microliters of vehicle, cells, or EVs were injected to the peri infarct area in 3 locations at 3 to 4 microliters per site. Intrathecal injections were performed using a 25G hypodermic needle and an insulin syringe (0.5 mL), 40 microliters of vehicle, cells, or EVs were injected between the last lumbar vertebra and the 1st sacral vertebrae (L6-S1).

The Body Swing Test was performed on day 1, 7, 14, 21, and 28 post-injury, and animals were sacrificed after testing 28 days post-injury. At twenty-eight days (Day 28) after MCAO, rats were anesthetized deeply with ketamine/xylazine (91 mg/kg ketamine, 9 mg/kg xylazine, respectively). After the rats were in the deep anesthetized stage, they were perfused transcardially with normal saline (with heparin 2 unit/ml) followed by 4% paraformaldehyde. Brains were removed and stored in 4% paraformaldehyde for 24 hours then changed to 1×PBS and stored in 0-4° C. All data were expressed as mean f S.E.M. The Body Swing Test data was analyzed by two-way ANOVA and Tukey's multiple comparison test. Significance is represented as *p<0.05, **p<0.01, ***p<0.001, ****p<0.0001.

The effects of the HMCs and HMC-EVs of the presently disclosed subject matter on locomotion were evaluated in MCAO models.

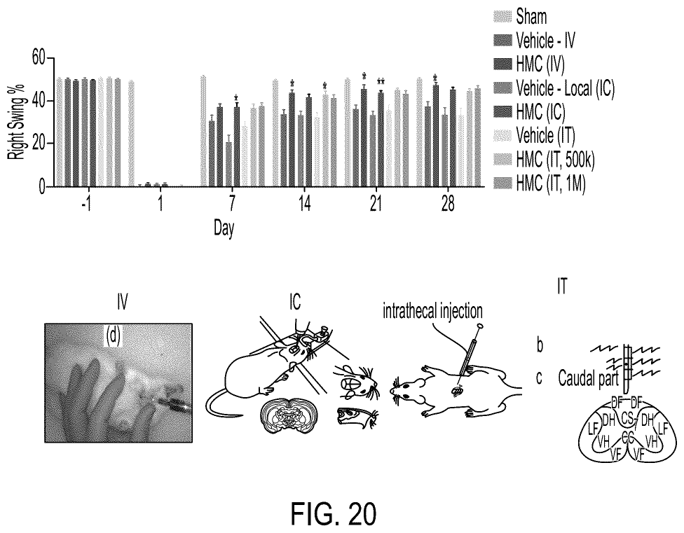

HMC cells were injected via three routes of administrations including intravenous (IV), intracerebral (IC) and intrathecal (IT) administration. Cells were dosed at 4 million in 0.5 mL per IV jection; 400,000 in 10 microliters per IC injection; and 500,000 or 1 million in 40 microliters per IT injection. As shown in FIG. 20, all treatment groups demonstrated improvement in recovering deficits in the Body Swing Test, with the IV and IC treatments having the most significance.

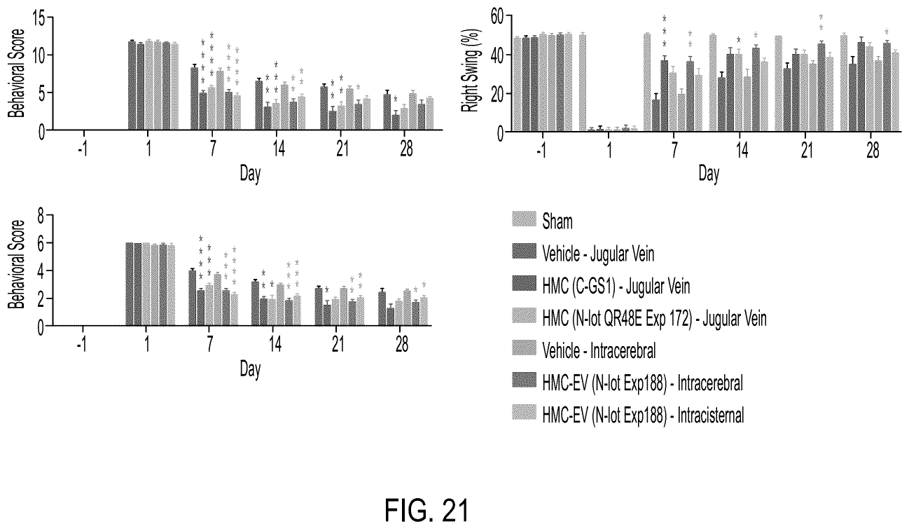

In another study, animals were subjected to the MCAO injury as described above. Cell treatments were administered on day 1 and day 7 after the MCAO surgery (24 hours and 7 days+/−10%) using HMCs, specifically HMCs derived from C-GS1 cells (C-GS1-HMC) and N-lot QR57 cells (N-HMC). The dosing of the cells was 4 million in 0.5 mL per IV injection. Extracellular vesicle (EV) treatments were administered on day 1 and day 7 after the MCAO surgery (24 hours and 7 days+/−10%) using EVs derived from N-HMCs (N-HMC-EVs). The dosing of the EVs was 10×10¹⁰ for intracerebral and intracisternal. All treatment groups demonstrated significant improvement in the limb placement tests (FIG. 21). In the Body Swing Test, all treatment groups provided recovery, with the C-GS1-HMCs, N-HMCs, and N-HMC-EVs via intracerebral injections demonstrating significant increases.

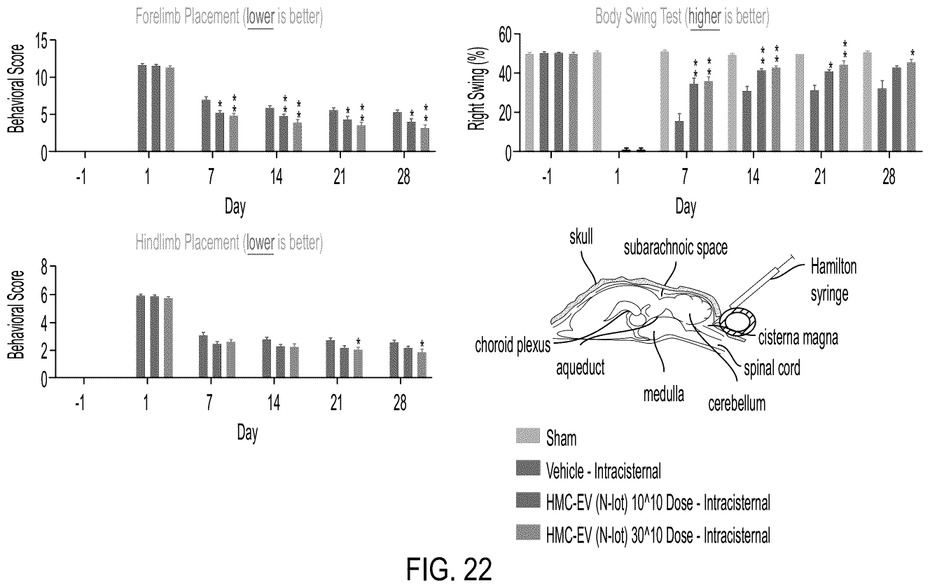

In a separate study, treatments were administered on day 1 and day 7 after the MCAO surgery (24 hours and 7 days+/−10%) using N-HMC-EVs (N-lot p6 and p7 treated with IFNgamma for 96 hours at 50 ng/ml). The dosing of the EVs was 10×10¹⁰ or 30×10¹⁰ total for N-HMC-EVs (stimulated N-lot) via intracisternal injections. All groups provided significant improvement in all three behavioral tests, with the most significant improvement demonstrated in the forelimb placement test and the body swing test (FIG. 22).

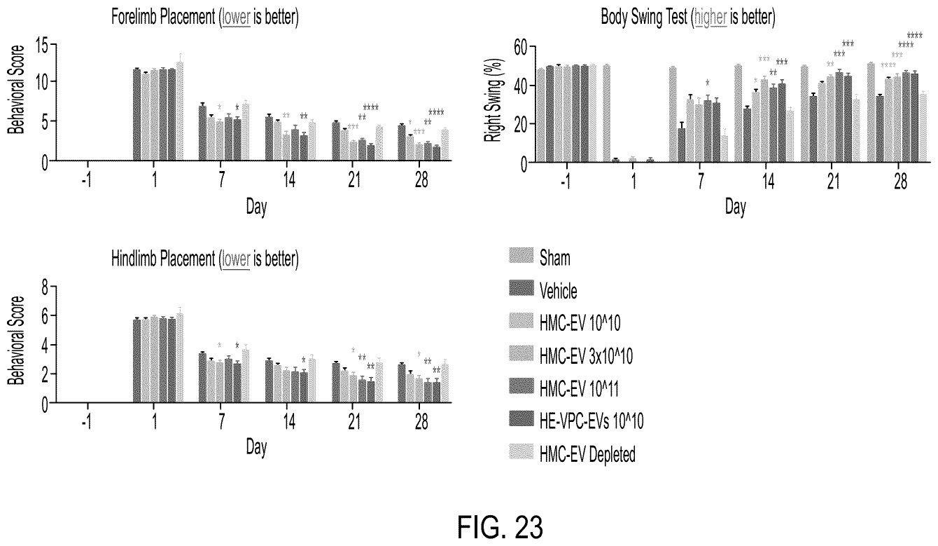

In yet another study, treatments were administered on day 1 and day 7 after the MCAO surgery (24 hours and 7 days+/−10%) using HMC-EVs (N-lot) or HE-VPC-EVs. The dosing of the exosomes was 10×10¹⁰, 30×10¹⁰, and 10×10¹¹ for HMC-EVs and 10×10¹⁰ for VPC-EVs via intrathecal injections. HMC-EV depleted injections were performed as a negative control. All groups provided significant improvement in all three behavioral tests, with the most significant improvement demonstrated in the forelimb placement test and the body swing test (FIG. 23).

Accordingly, the HMCs of the presently disclosed subject matter and HMC-EVs were efficacious in an MCAO stroke model via intravenous, intrathecal, intracerebral and/or intracisternal administrations, and both HMC and EV treatments provided improved locomotor recovery in behavioral tests.

### In Vivo Effects of HMC on Histopathological Outcome

The effects of the HMCs of the presently disclosed subject matter on histopathological outcome were assessed. Specifically, animals were subjected to the MCAO injury as described above. Cell treatments were administered on day 1 and day 7 after the MCAO surgery (24 hours and 7 days+/−10%) using HMCs, specifically HMCs derived from C-GS1 cells (C-GS1-HMCs) and N-lot QR57 cells (N-HMCs). The dosing of the cells was 4 million in 0.5 mL per IV injection.

Sham, vehicle, and cell treatment groups were prepared for histopathological analysis for white matter loss (MBP), and markers for neuroinflammation such as microglial activation (Iba-1) and astrocyte activation (GFAP).

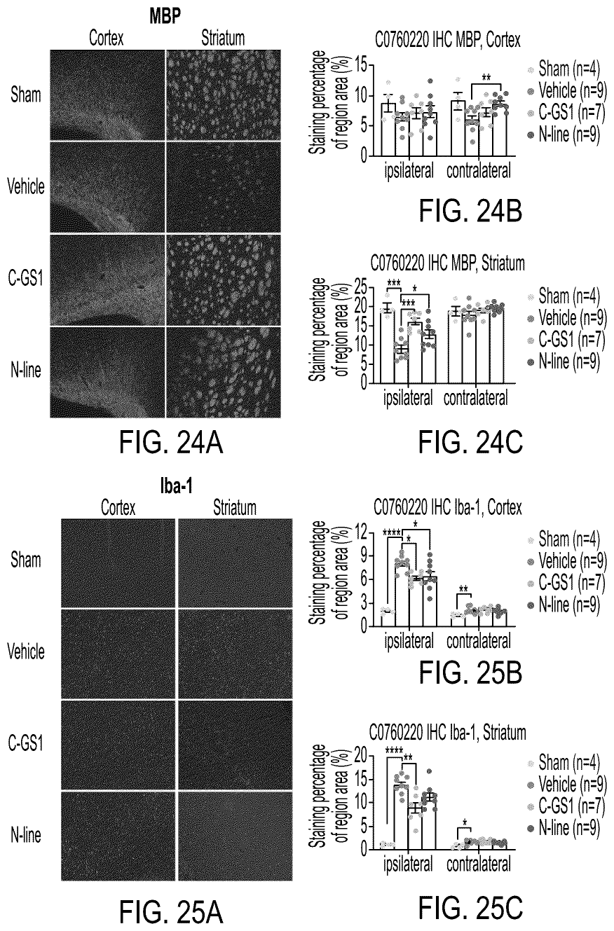

FIG. 24 shows preservation of myelin with HMC cell treatment in striatum. Specifically, for MBP, there was a statistically significant difference between the sham and vehicle, but there was no statistically significant difference between the vehicle and treatment groups in the ipsi part of the cortex. There was a statistically significant difference between the vehicle and N-line cell treatment groups in the contralateral cortex, however, there was no statistically significant difference between the groups in the ipsi and as well as sham and vehicle in the contra part of the cortex. There was a statistically significant difference between the sham and vehicle for both ipsi and contra in striatum, vehicle and both cell treatment groups only in ipsi part of the striatum. There was no statistically significant difference between the groups in the contra part of striatum.

FIG. 25 shows reduced microglial activation following HMC administration. Specifically, for Iba-1, there was a statistically significant difference between the sham and for both ipsi and contra part of cortex, vehicle and cell treatment groups only in ipsi part of cortex. There was no statistically significant difference between the vehicle and treatment groups in the contra part of cortex. There was a statistically significant difference between the sham and vehicle for both ipsi and cotra part of striatum, vehicle and C-GS1 cell treatment groups in the ipsi part of striatum. There was no statistically significant difference between the vehicle and treatment groups in the contra part of striatum.

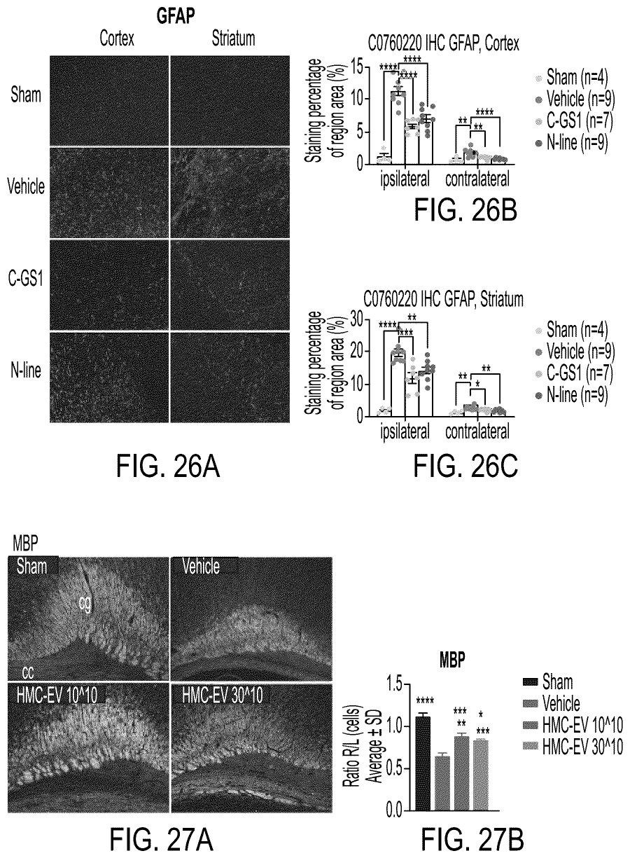

FIG. 26 shows reduction of astrocyte reactivity upon HMC treatment. Specifically, for GFAP, there was a statistically significant difference between the sham and vehicle as well as vehicle and cell treatment groups for both ipsi and contra part of cortex. There was a statistically significant difference between the sham and vehicle as well as vehicle and cell treatment groups for both ipsi and contra part of striatum.

Accordingly, these results demonstrated that the MSCs of the presently disclosed subject matter not only increased preservation of myelin, thus white matter, but also resulted in robust reduction of neuroinflammation markers by reducing the number of reactive astrocytes and microglia.

In vivo effects of HMC-EVs on histopathological outcome

The effects of HMC-EVs on histopathological outcome were also assessed. Specifically, animals were subjected to the MCAO injury as described above. Treatments were administered on day 1 and day 7 after the MCAO surgery (24 hours and 7 days+/−10%) using HMC-EVs (N-lot p6 and p7 treated with IFNgamma for 96 hours at 50 ng/ml). The dosing of the EVs was 10×10¹⁰ or 30×10¹⁰ total for HMC-EVs (stimulated N-lot) via intracisternal injections.

Sham, vehicle, and cell treatment groups were prepared for histopathological analysis for MBP, Iba-1, GFAP, Olig-2, and NG2. FIG. 27 shows preservation of myelin with intracisternal delivery of EVs. Specifically, MBP IF staining showed a stable stained area in all treatment groups in the range of 0.81-0.88. The mean ratio of the vehicle group was the lowest (0.64). The differences between the vehicle group and all the treatment groups were significant.

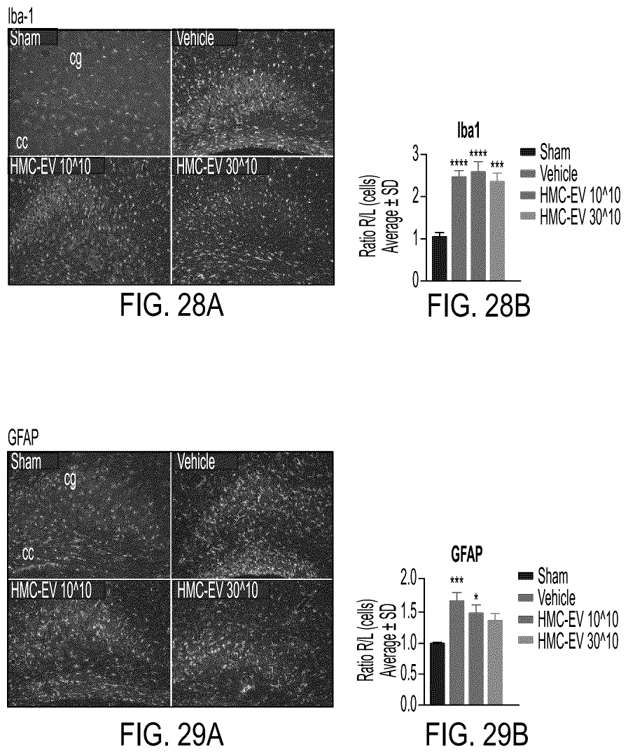

FIG. 28 shows the effects of HMC-EV treatment on microglial activation. Specifically, Iba-1 IF staining showed the same mean ratio (R/L) of the number of positive cells in the vehicle group and in the HMC-EV 10¹⁰ and HMC-EV 30¹⁰ treatment groups (˜2.5).

FIG. 29 shows the effects of intracisternal HMC-EV delivery on astrocyte reactivity. Specifically, GFAP IF staining did not reveal any differences between the control and all treatment groups and showed stable mean ratios (R/L) of the number of positive stained cells.

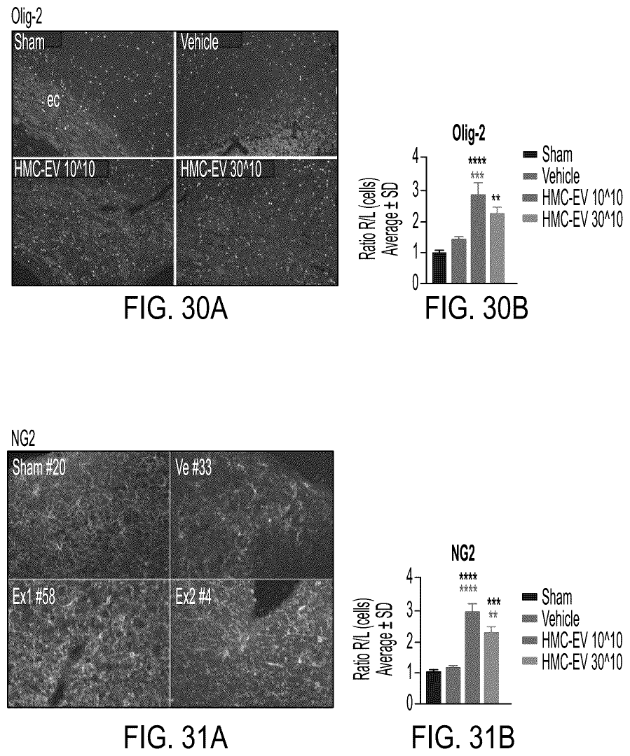

FIG. 30 shows that intracisternal delivery of HMC-EVs increased oligodentrocytes. Specifically, Olig-2 IF staining revealed highest mean ratio (R/L) of positive stained cells in all exosome treatment groups (compare to the vehicle group). The differences between the Vehicle group and HMC-EV 10¹⁰ and HMC-EV 30¹⁰ were significant.

FIG. 31 shows that intracisternal delivery of HMC-EVs increased oligodentrocyte precursor cells. Specifically, NG2 IF staining revealed a statistically significant increase in the mean ratio (R/L) of positive stained area in HMC-EV 10¹⁰ and HMC-EV 30¹⁰ compared to the vehicle group.

Accordingly, these results demonstrated that HMC-EVs increased preservation of myelin. In addition, EV treatment also increased oligodentrocytes and oligodentrocyte-precursor cells.

### Example 8—In Vitro Oxygen Glucose Deprivation Stroke Model

The neuroprotective effect of MSCs of the presently disclosed subject matter was examined in vitro. An oxygen glucose deprivation (OGD) assay which combines hypoxic conditions with glucose-deprived media was used to model stroke in vitro.

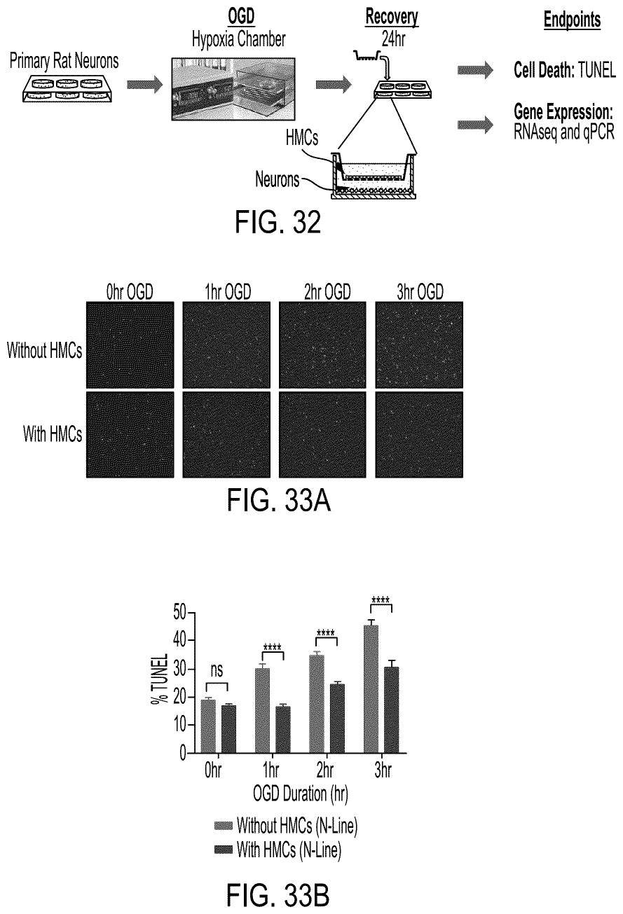

The overview of the assay is shown in FIG. 32. For primary neuronal culture, embryonic day 18 (E18) rat cortex samples (#SDECX), sourced from Sprague Dawley rats, were ordered from Brain Bits, LLC (Springfield, IL). The cortices were washed in dissection media (DM) three times. DM consists of 50 mL 10×HBSS (w/o Ca and Mg; Gibco 14185-052), 500 μL Gentamicin, 5 mL pyruvate (Gibco: 11360070), 5 mL Hepes (Gibco 15630080) 10 mM final, 15 mL Glucose 30 mM Final (1M stock), and 425 mL water. After washing, DM was aspirated and the tissue was then minced into equal sized pieces with scalpel. A DM, papain, and DNase I solution was prepared while washing tissue by measuring 1 mL DM, 40 uL papain (Worthington LS003126), and 2.5 uL DNase I (DNase (Sigma #DN-25) per brain; activating the papain with incubation in a 37° C. water bath for 30 minutes; and sterile filter using a 0.22 micron filter. The DM, activated papain, and DNase I solution was added to the cortex samples and incubated at 37° C. for 30 minutes to dissociate the tissue.

During this time, neuronal media (NMO) was also prepared and incubated at 37° C. NMO consists of Neurobasal plus media with 1× B27 plus added fresh (Neurobasal Plus and B27; Life Tech Corp A3653401), 1× Glutamax (Gibco #35050-061), and gentamycin sulfate (MP Biomedical #0916760-CF). Dissociation pipets were prepared by fire polishing Pasteur pipets with sequentially smaller tip diameters (1=just flame polish, 2=3/4 of original diameter, 3=1/2 of original diameter. After the 30 minutes incubation, the tissue was removed from the water bath. The DM/papain/DNase I solution was gently aspirated and 5 mL of pre-warmed NMO with freshly added B27 was added. The tissue was allowed to settle, and the NMO was gently pipetted off. The tissue was washed again with 5 mL fresh NMO (with B27), and this was repeated for a total of 3 washes. After the last wash, the NMO was removed. The tissue was dissociated by gently triturating the brain tissue through a fire-polished Pasteur pipet, starting with the largest pipet. This was performed by adding 3 mL of NMO, gently triturate 4-5X, and dispensing tissue against wall of tube to prevent bubble formation as neurons trapped in bubbles will die. After the remaining tissue settled, the supernatant was removed and added to a fresh 50 mL falcon tube. This was repeated for all pipet sizes and the cell mixture was then passed through a 70 micron cell strainer. Cells were counted and diluted to 600,000 cells per mL. Cells were plated on tissue culture plates precoated with poly-D-lysine (PDL). For a 6-well plate, 2 mL was added for a total of 1.2 million cells per well. For a 24-well plate, 0.5 mL was added for a total of 300,000 cells per well. Cultures were then fed with ½ media changes every 3rd day to prevent metabolic byproduct accumulation. After one week, the cells were then subjected to the OGD assay.

Five days before the endpoint processing for the neurons, N-lot HMC were thawed in a 37° C. water bath with gentle swirling. Once thawed, cells were pipetted dropwise into pre-warmed MSC media (alpha MEM without nucleosides (Hyclone, #SH30568.01), 20% Defined FBSHeat Inactivated (Hyclone, #SH30070.03HI), 1× Glutamax (Gibco #35050-061), 1×MEM NEAA (Gibco #11140-050), 1× Pen/Strep (Gibco #15140-120)). Cells were then centrifuged at 300×g for 5 min, resuspended, and counted. 1 million MSCs were plated in a T225 flask using 50 mL of MSC media and allowed to persist in culture for 4 days. HMCs were then harvested by first aspirating the media. The flask was washed with 10 mL of PBS, the PBS was aspirated, 3 mL of TrypLE Express (Gibco, #12604021) was added, and the cells were incubated at 37° C. for 4-6 minutes. Following the incubation, the cells were washed with MSC media, collected into a 50 mL conical tube, the plate was washed with MSC media to remove remaining cells, the cells were centrifuged for 5 minutes at 300×g. The cells were then resuspended in MSC media and counted. HMCs were then plated in transwell inserts in MSC media to achieve a 1:10 ratio of HMCs to neurons (for 6-well transwell inserts, 120,000 HMCs were plated per well, and for 24-well transwell inserts, 30,000 HMCs were plated per well). The HMCs were allowed to recover for 24 hours, and the MSC media was replaced with NMO to remove traces of FBS. The HMCs were incubated in NMO media for 24 hours until their use for recovery in the oxygen glucose deprivation (OGD) assay.

For the OGD assay, OGD media was used to deprive the neurons of glucose. OGD media consisted of 1 mM CaCl₂), 5 mM KCl, 137 mM NaCl, 0.4 mM KH2PO4, 0.3 mM Na₃HPO₄, 0.5 mM MgCl₂, 0.4 mM MgSO₄, 25 mM HEPES, 4 mM NaHCO₃, 1× Pen/Strep diluted in 450 mL DI water. The pH was adjusted to 7.3 and water was added for a final volume of 500 mL. The media was then sterile filtered using a 0.2 μm filter. One day prior to initiating the OGD experiment, OGD media was placed in T75 vented flasks and incubated in a hypoxia chamber (C-Chamber with ProOx C21 Oxygen CO₂ Single Chamber Controller, BioSpherix, Parish, NY) overnight to allow for diffusion of oxygen out of the media. The next day, the OGD media was removed from hypoxia chamber and neurons were washed once with OGD media to remove traces of NMO. OGD media was removed and a complete media change with OGD media was performed just prior to adding cells to chamber, i.e. media for 3 hr OGD duration was changed, but media for 2 hr time point was not changed until just before adding cells to chamber, etc. This ensures that the recovery time was the same for all conditions. Neurons were incubated in the hypoxia chamber with OGD media for 1, 2, or 3 hours. Once finished, the neurons were removed and complete media change with NMO media (+B27) was performed. For noninjured controls, NMO was replaced with OGD media, but neurons were not incubated in hypoxia chamber. OGD media in the non-injured controls was replaced with NMO at the same time as the injured cells. HMC co-culture conditions were performed for both non-injured controls and injured cells. Immediately after the OGD media was replaced with NMO, the transwell inserts with HMCs were added in the co-culture conditions. Recovery from the OGD injury was allowed to persist for 24 hours in an incubator under normal cell culture conditions. The neurons were either collected for RNA isolation, or fixed and subjected to TUNEL staining.

### In Vitro OGD Assay TUNEL Analysis

Primary neuronal culture was generated from embryonic day 18 (E18) rat cortex samples, sourced from Sprague Dawley rats, that were ordered from Brain Bits, LLC (Springfield, IL) as described above. HMC co-culture conditions using a transwell insert (no direct contact) at a ratio of 1:10 HMCs to neurons were performed using N-lot cells, and initiated immediately after OGD injury for a total duration of 24 hrs.

To assess the effects of HMC co-culture to prevent neuronal cell death caused by the OGD assay, TUNEL staining, imaging, and quantification was performed. After the OGD assay, the transwells were removed in co-culture conditions, and the neurons were first fixed with 4% paraformaldehyde. To fix the cells, the NMO was removed and 4% paraformaldehyde was applied to each well and incubated at room temperature for 10 minutes. After the fixation, the cells were then washed 3× with PBS and permeabilized with 0.02% Triton-X in PBS for 10 minutes at room temperature. The cells were then washed 3× with PBS. The positive control was designated and treated with DNase I (Sigma #4536282001) in DNase I Reaction Buffer (20 mM Tris-HCl, pH 8.4, 2 mM MgCl₂, 50 mM KCl) for 30 minutes at room temperature at 370 for 30 minutes. The positive control was then washed 3× in PBS.

To achieve TUNEL staining, the TUNEL Label Mix (Sigma #11767291910) and TUNEL Enzyme kit (Sigma #11767305001) was used according to the manufacturer's protocol with slight variation. In general, two kits were used per experiment and diluted in PBS to accommodate the larger volume for 24-well plates. The instructions suggest to use the kit directly with a volume of 50 uL per well, but to ensure coverage of a 24-well plate, PBS was used to dilute the sample for 150 uL per well. For negative control, TUNEL labeling reagent without TUNEL enzyme diluted in PBS was used. For all samples, 200 μL of DAPI staining solution (VWR #10791-650) was added to the combined solution. TUNEL labeling reagent with TUNEL enzyme dilution was added to desired wells, and samples were incubated for 1 hr at 37° C. Samples were washed 3× with PBS. Imaging was performed on the Leica DMi8 microscope and quantification was performed using the Leica LAS X Navigation software. For each condition, 3 wells were stained and 9 images per well were taken and quantified, producing 27 images per condition to be analyzed. TUNEL staining and analysis demonstrated significant increase in cell death with increasing OGD injury duration.

As shown in FIG. 33, HMC co-culture prevented cell death in primary rat neurons following OGD injury. Neuroprotective effects of HMC cells in ischemic injury do not require direct contact with neurons, function via paracrine effect onto target neurons.

Accordingly, the in vitro analysis demonstrated that HMCs of the presently disclosed subject matter can protect from ischemic injury (i.e., oxygen glucose deprivation) in isolated neuronal culture preparations, demonstrating a benefit of direct access to central nervous system in stroke.

### RNAseq Analysis of Oxygen-Glucose Deprived Rat Neurons

Primary rat neuronal culture was subjected to oxygen glucose deprivation (OGD) for various durations (e.g., 0, 1, 2 and 3 hours injury duration). Neurons were subsequently co-cultured with HMCs for 24 hours after OGD treatment. RNA samples were collected 24 hours after OGD treatment. RNA-seq analysis was performed to examine transcriptome and pathway enrichment following OGD in vivo injury with or without subsequent HMC co-culture.

For RNA isolation, neurons were collected by washing with PBS, scraping, and centrifuging in a microcentrifuge tube at 500 g for 5 minutes. The PBS was aspirated and the cell pellet was either snap frozen and placed at −80° C. or immediately processed through the RNeasy RNA isolation kit (Qiagen #74104) following the manufacturer's protocol. RNA was quantified using a Nano Drop and all samples were normalized to 50 ng/uL and 1 ug total was submitted to GeneWiz for RNAseq analysis with the goal of analyzing the changes in gene expression in response to the OGD injury and HMC co-culture. The conditions were Control, Control with HMCs, 1 hr OGD, 1 hr OGD with MSCs, 2 hr OGD, 2 hr OGD with MSCs, 3 hr OGD, and 3 hr OGD with HMCs. For each condition, 3 biological replicates were provided.

Library preparation was performed using the NEB Ultra II RNA library preparation kit followed by Illumina sequencing. For each sample, 20-30 million reads were achieved. Bioinformatic analysis was performed, and RNAseq data was analyzed. Reads were trimmed using cutadapt1. Quality scores were assessed using FastQC2. Reads were aligned to the Rattus norvegicus genome build rn6 using STAR3. Individual sample reads were quantified using HTseq4 and normalized via Relative Log Expression (RLE) using DESeq2 R library5. Read Distribution percentages, violin plots, identity heatmaps, and sample MDS plots were generated as part of the QC step using RSeQC6. DEseq2 was also used to calculate fold changes and p-values and perform optional covariate correction. Clustering of genes for the final heatmap of differentially expressed genes was done using the PAM (Partitioning Around Medoids) method using the fpc R library7. Hypergeometric distribution was used to analyze the enrichment of pathways, gene ontology, domain structure, and other ontologies. The topGO R library8, was used to determine local similarities and dependencies between GO terms in order to perform Elim pruning correction. Several database sources were referenced for enrichment analysis, including Interpro, NCBI, MSigDB REACTOME, WikiPathways. Enrichment was calculated relative to a set of background genes relevant for the experiment. Although numerous gene expression changes were observed, genes involved in neuroprotection were highlighted.

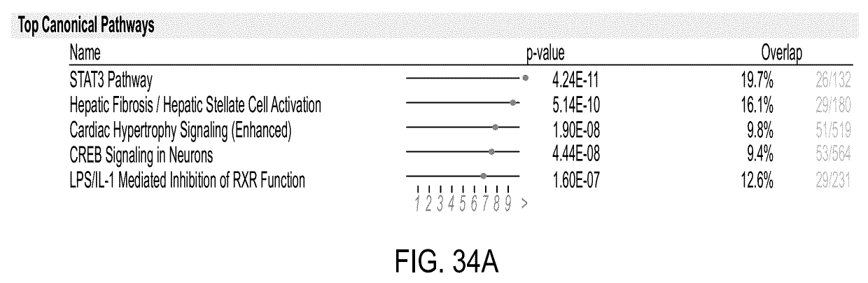

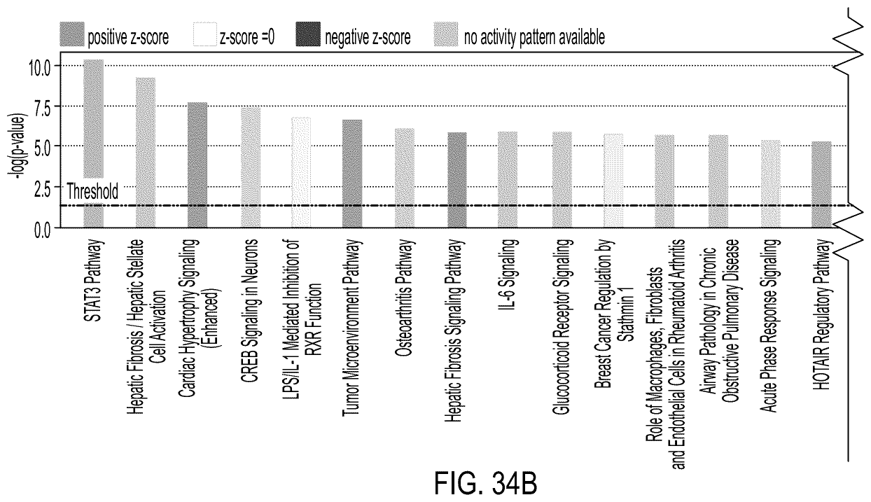

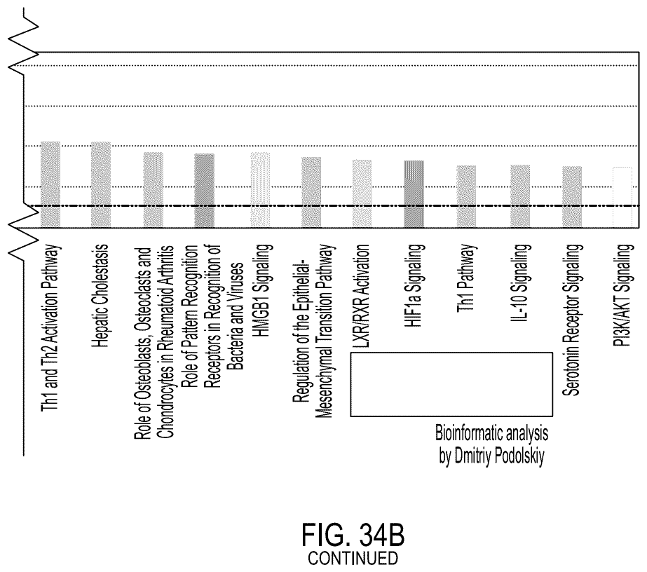

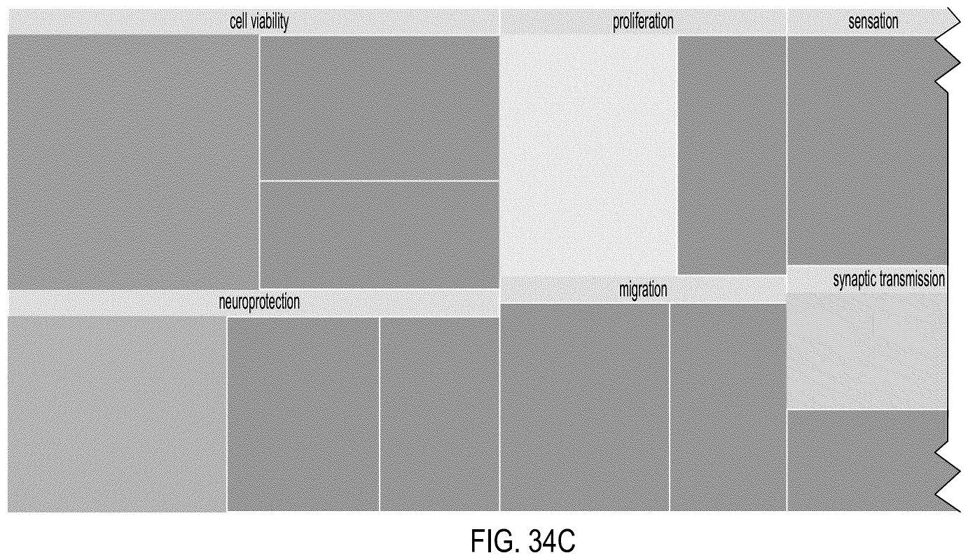

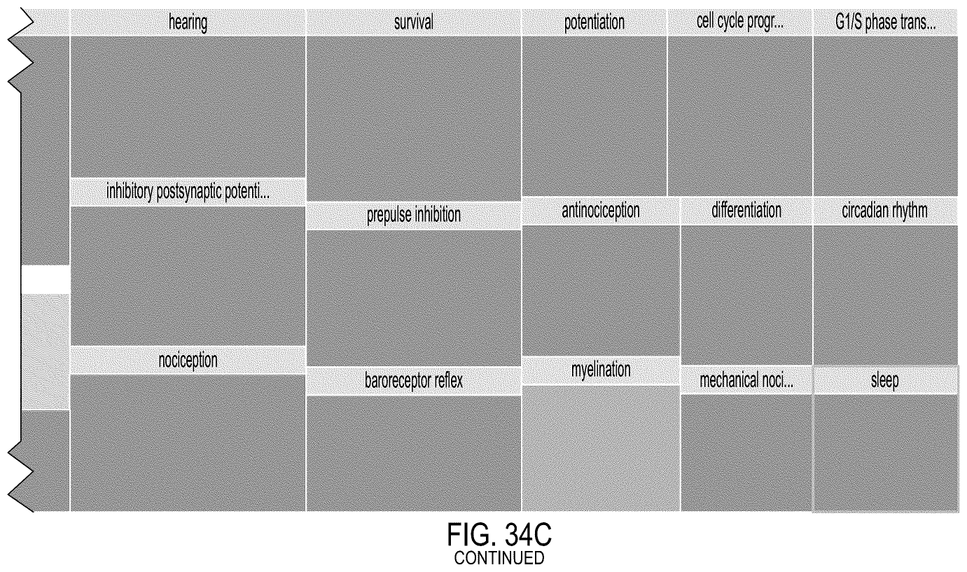

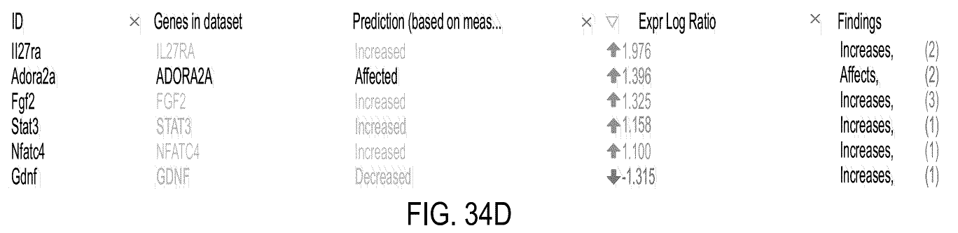

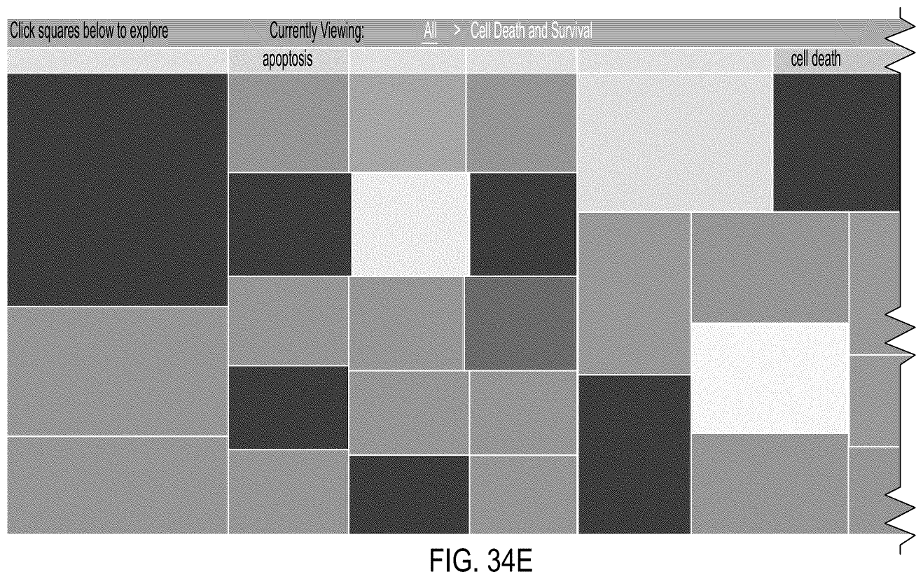

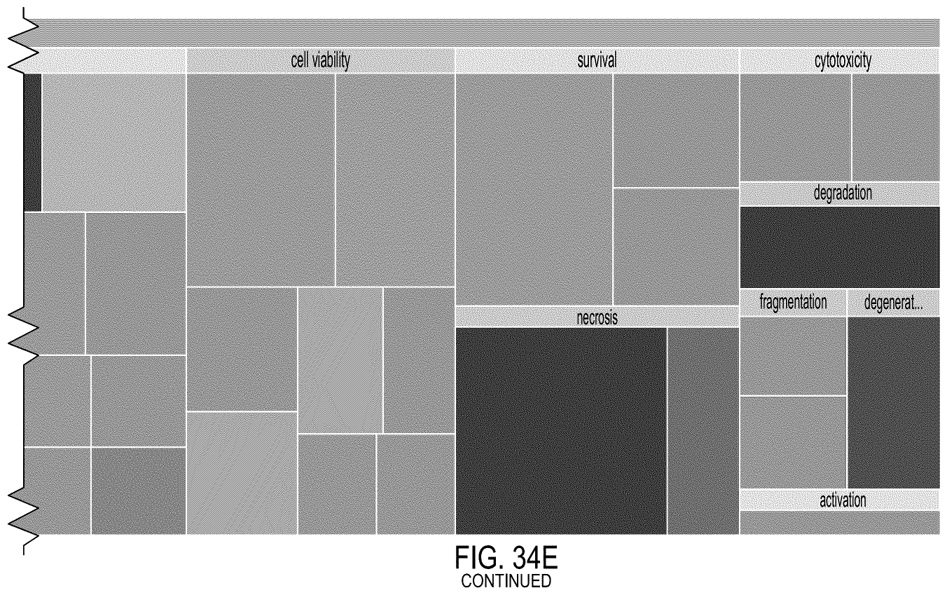

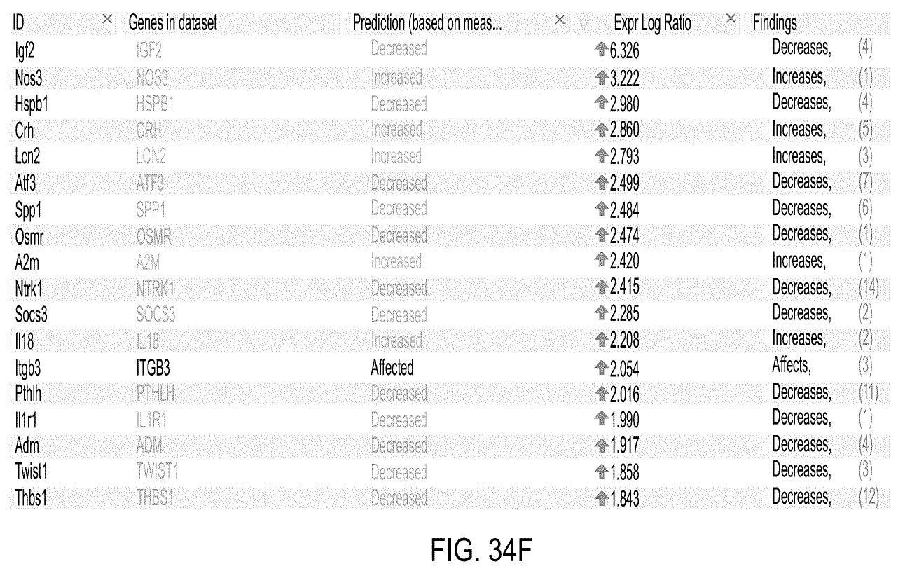

The therapeutic effect of HMC-enriched culture for OGD neuron growth was observed for neurons subjected to 3 hours of OGD damage. Pathway enrichment analysis of the differential expression between neurons subjected to 3 hours of OGD damage and grown on HMC-enriched and control media was performed using Qiagen Ingenuity Pathway Analysis framework. As shown in FIGS. 34A-C, pathways enriched by this differential expression include (a) STAT3 pathway (p-value: 4×10⁻¹¹), deactivated in HMC-cultured OGD neurons, (b) CREB signaling in neurons (p-value: 4.4×10⁻⁸), and (c) numerous inflammatory activity pathways downregulated in HMC-cultured OGD neurons (e.g., IL-6 signaling, IL-10 signaling, Th1/2 activation pathway).

Enriching differential expression between OGD neurons grown on HMC-enriched and control media for Gene Ontologyterms (FIGS. 34C-F) in turn shows increase in cell viability of OGD neurons grown on HMC-enriched culture (FIG. 34C), direct neuroprotective effect (FIG. 34C, genes involved in upregulation of neuroprotection are presented on FIG. 34D) and upregulation of pathways involved in synaptic transmission (FIG. 34C). Simultaneously, pathways involved in apoptosis (FIG. 34E, genes downregulated by the effect of HMC-enriched growth culture are presented on FIG. 34F) and general response to cell death are strongly downregulated. This reflects the relation between full differential expression and the displacement of the molecular marker of OGD damage induced by the presence of HMC-enriched growth medium.

To validate these increases in gene expression, the same RNA samples used for RNAseq analysis were used for qPCR analysis. To perform qPCR analysis, Taqman probes (ThermoFisher Scientific) were designed and used with the Taqman Fast Advanced Master Mix (ThermoFisher Scientific #4444556) and samples were analyzed on the QuantStudio Flex 7 RT-PCR system (Applied Biosystems #4485698). The three biological replicates for each sample were run in duplicate, and the analysis demonstrates the similar increase in gene expression with the presence of HMCs. Statistical significance was achieved through 2-way ANOVA and Sidak multiple comparison test (* p<0.05, ** p<0.01, **** p<0.0001).

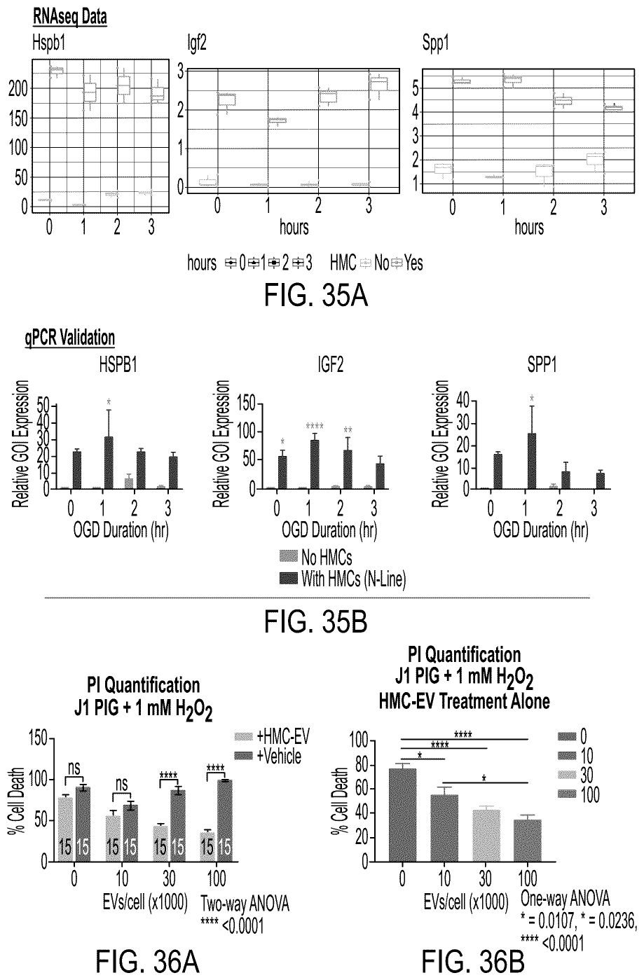

As shown in FIG. 35, qPCR analysis verified RNAseq results of genes involved in cell viability and neuroprotection. Specifically, HMC cells stimulated expression of neuroprotective genes in neuron undergoing ischemic injury, such as heat shock protein family B member 1 (HSPB1), insulin-like growth factor 1 (IGF2), and secreted phosphoprotein 1 (SPP1), also known as osteopontin.

### Example 9—In Vitro Oxidative Damage Model

The HMC-EVs of the presently disclosed subject matter were tested in an in vitro oxidative damage model. Briefly, neurons were subject to H₂02 oxidative damage, and treated with HMC-EVs at a dose of about 10,000, 30,000 or 100,000 EVs/cells. Percentage of cell death was determined as the number of propidium iodide (PI)-positive cells out of the total cell number.

As shown in FIG. 36, HMC-EV treatment resulted in a dose-dependent attenuation of cell death. A significant rescue from cell death by HMC-EVs was observed at 30K and 100K doses. The overall cell death rate was about 44% lower than the control group without EV treatment.

Accordingly, these results demonstrated that HMC-EVs can prevent oxidative injury in neurons.

### Example 10—In vitro Glutamate Excitotoxicity Model

The HMC-EVs of the presently disclosed subject matter were tested in an in vitro glutamate excitotoxicity (high doses of L-glutamate) model. Briefly, neurons were exposed to various concentrations of L-glutamate (about 0, 30, 300 and 3000 uM), and treated with HMC-EVs at a dose of about 50,000 EVs/cells. Percentage of cell death was determined as the number of propidium iodide (PI)+ cells out of the total cell number.

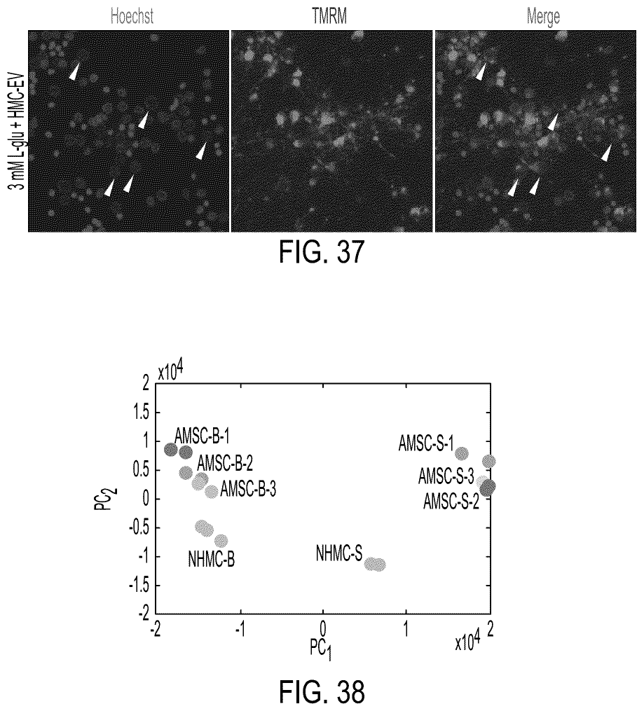

As shown in FIG. 37, HMC-EV treatment sustained cells in the nuclear swelling stage after glutamate-induced injury and maintained viability. Staining with TMRM (cell permeant dye that accumulates in active mitochondria with intact membrane potentials) showed that HMC-EV treatment also maintained mitochondrial activity in injured cells.

Accordingly, these results demonstrated that HMC-EVs prevent neuronal death due to glutamate excitotoxic injury.

### Example 11—RNAseq analysis of HMCs vs Bone Marrow-MSC vs Adipose Tissue-MSC

RNAseq analysis was performed for the HMCs of the presently disclosed subject matter under both basal and stimulated conditions. HMCs were generated from both N-line (N-HMCs) and GMP-1 (GMP-HMCs) cell line, and 3 technical replicate samples were prepared for each condition. MSCs isolated from adipose tissue and bone marrow were also analyzed and compared with the HMCs of the presently disclosed subject matter. AD-MSCs were collected from 3 different adult donors, and 2 technical replicate samples were prepared for each biological replicate. BM-MSCs were also collected from 3 different adult donors.

HMCs Vs. Adipose Tissue Derived MSCs

Principal component analysis of transcriptomes of HMCs (obtained from the N-cell line) and AD-MSCs shows that HMCs are distinct from the latter in both basal and interferon-gamma stimulated state (FIG. 38). The first principal component largely describes the effect of stimulation with gamma interferon, while the second principal component describes the difference between HMCs and AD-MSCs.

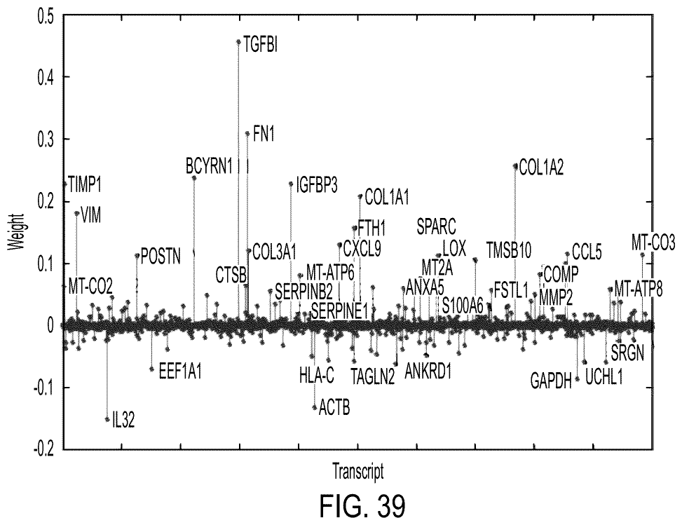

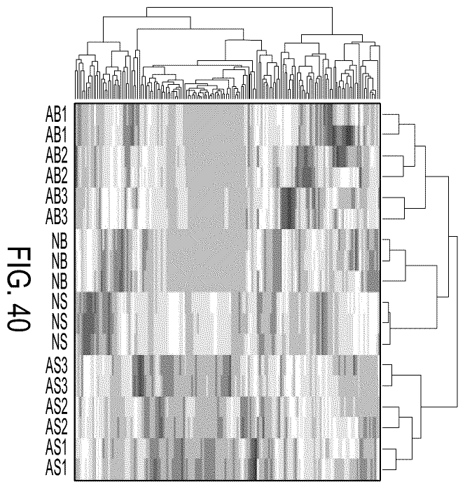

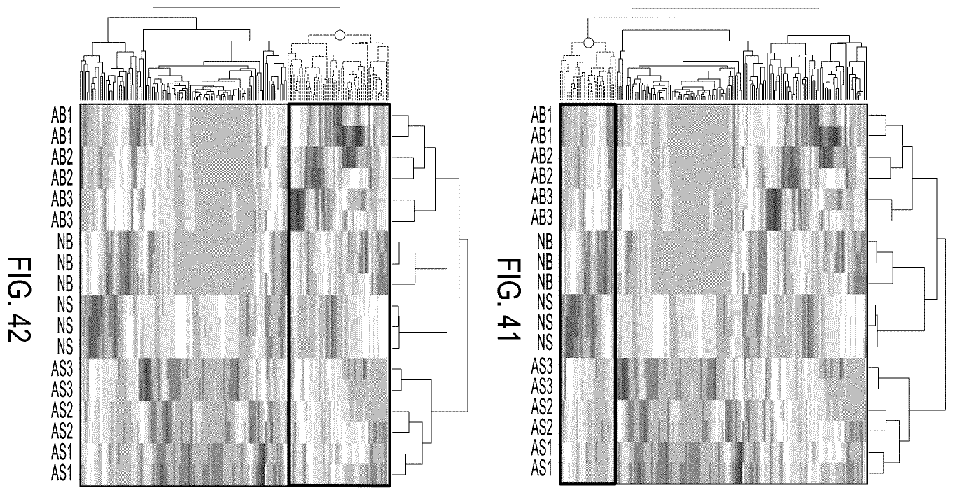

Weights of different genes contributing to the second principal component which determines the variance between HMCs and AD-MSCs. Of a particular note is down-regulation of collagen genes (COLlA1, COL3A1 etc.), mitochondrial function genes and TGF Beta 1 (one of the main factors promoting angiogenesis) in HMCs as compared to AD-MSCs demonstrating a certain degree of immaturity of HMCs (FIG. 39).

Hierarchical clustering demonstrates similarity between biological/technical replicate samples of the same biological type as well as clear difference between HMCs and AD-MSCs, in both basal cell states and cell states stimulated with gamma interferon (FIG. 40).

As shown in FIG. 41, genes in this cluster were up-regulated in HMCs (both basal and INFN gamma-stimulated) as compared to AD-MSCs. The genes included: CALR, UBB, PKM, CXCL8, C15orf48, PSME2, TPM3, ANKRD1, PFN1, SRGN, ACTB, MDK, TAGLN2, CFL1, HSP90AA1, HSPA8, CXCL12, UCHL1, HMGA2, HMGA1, HN1, PTMA, SP90AB1, PRDX1, GSTP1, KRT18, IGFBP4, CALD1, COL4A1, COL4A2 and GAPDH. Differential expression of these genes between HMCs and AD-MSCs was consistent across biological and technical replicates according to the hierarchical clustering map.

Functional annotation of biological pathways enriched in the cluster on FIG. 41 was performed using Reactome (https://reactome.org/). The top pathway enriched by the corresponding genes was associated with axon guidance. Other significantly enriched pathways included cellular stress response and developmental biology (related to the relative immaturity of HMCs).

As shown in FIG. 42, genes in this cluster were down-regulated in HMCs (both basal and INFN gamma-stimulated) as compared to AD-MSCs. The genes included: SERPINE1, ACTA2, TPM2, CTGF, SERPINE2, CRYAB, ELN, MFGE8, ANXA2, POSTN, VIM, MFAP5, ISLR, THBS1, TIMP3, DKK1, COL6A3, COL6A1, TPT1, BCYRN1, COL1A1, SPARC, TPM1, BGN, COL1A2, COL3A1, TGFBI, CRLF1, COMP, NEAT1, MT-CO3, MT-CO2, MT-ATP8, MT-CYB, MT-CO1, MT-ATP6, MT-ND4, MT-ND4L, MT-ND5, MT-ND6, MT-ND3, MT-ND1, MT-ND2, GREM1, TMSB4X, ITGB1, LMNA, H2AFZ, FTL, EEF1G, NPM1, EEF1A1, RACK1, ACTG1, and TPM4. Differential expression of these genes between HMCs and AD-MSCs was consistent across biological and technical replicates according to the hierarchical clustering map.

Functional annotation of biological pathways enriched in the cluster on FIG. 42 was performed using Reactome (https://reactome.org/). The top pathways enriched by the corresponding genes were associated with respiratory electron transport and mitochondrial function in general as well as collagen biosynthesis.

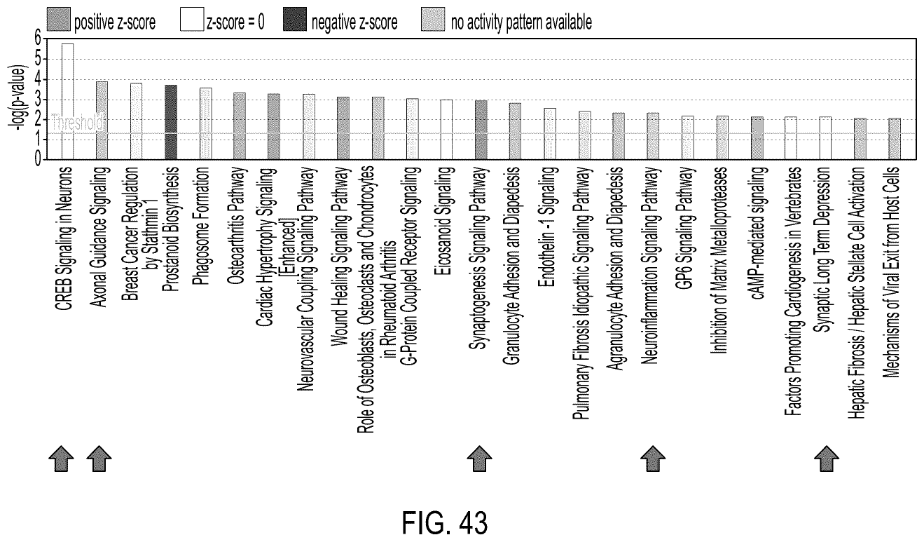

Canonical pathway enrichment of differential gene expression signature between HMCs and AD-MSCs shows noticeable HMC-specific up-regulation of several pathways (denoted by red arrows) involved in the development of neuronal lineage including axon guidance, CREB signaling in neurons, synaptogenesis signaling etc. (FIG. 43). These results suggest that HMCs have a distinct expression profile when compared to AD-MSCs, and HMCs may confer neuroprotective effects, and provide neurotrophic factors, factors involved in supporting neuronal health and recovery.

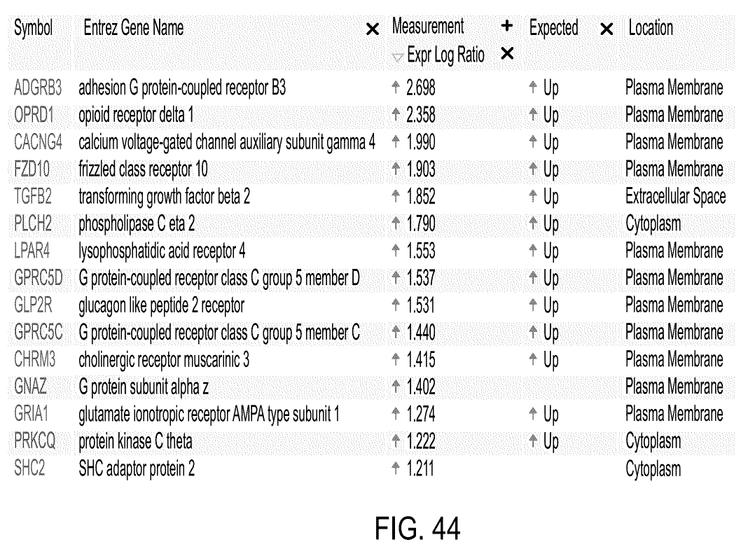

Lists of genes-contributors to the activated pathways establishing this difference are shown in FIGS. 44-47.

FIG. 44 depicts the top 15 most strongly differentially expressed genes contributing to activation of neuronal CREB signaling in HMCs. Expr Log Ratio denotes base 10 logarithm of the fold change between average TPM expression of a gene in HMCs and its average TPM expression in adipose tissue-derived MSCs, i.e., the Expr Log Ratio higher than 2 implies gene expression increase by a factor larger than 100.

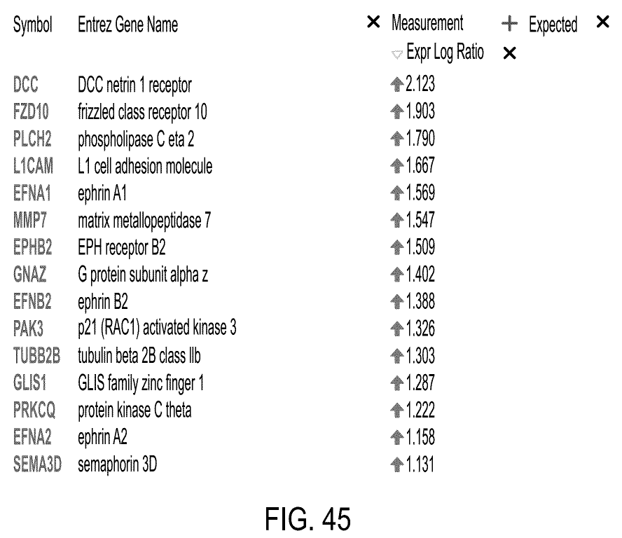

FIG. 45 depicts the top 15 most strongly upregulated genes contributing to the enrichment of axon guidance pathway in HMCs. Although activation pattern of axonal guidance signaling pathway has not been determined by Qiagen Ingenuity Pathway Analysis, the pathway was enriched with p-value ˜1.38e-4 in HMCs as compared to AD-MSCs.

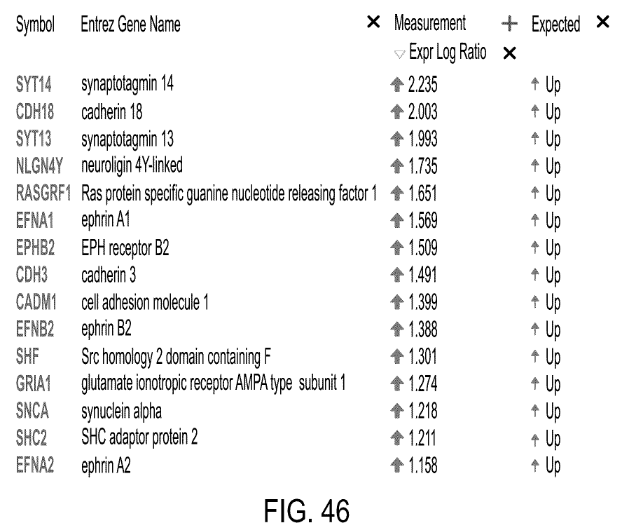

FIG. 46 depicts the top 15 most strongly expressed genes contributing to activation of synaptogenesis signaling pathway in HMCs. Enrichment p-value 1.14e-3, activation pattern z-score 3.578, the highest among all pathways differentially upregulated in HMCs.

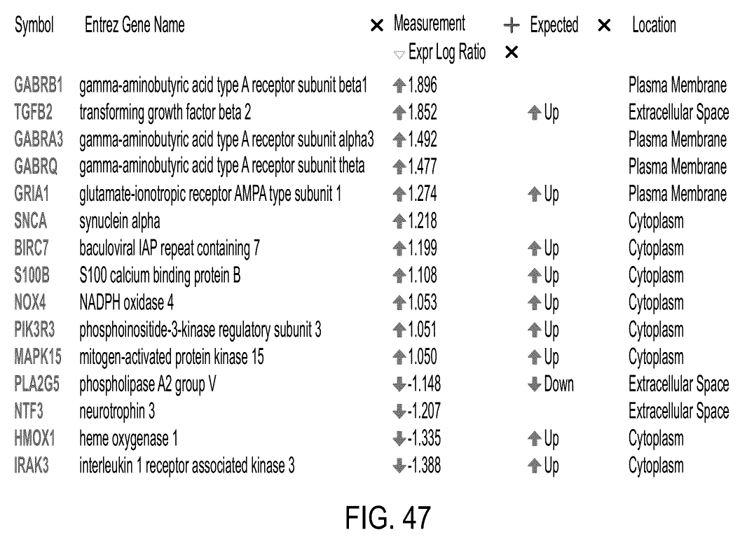

FIG. 47 depicts the top 15 most up-regulated genes out of contributing to activation of neuroinflammation signaling pathway in HMCs. Pathway enrichment p-value 4.97e-3, activation z-score 1.508.

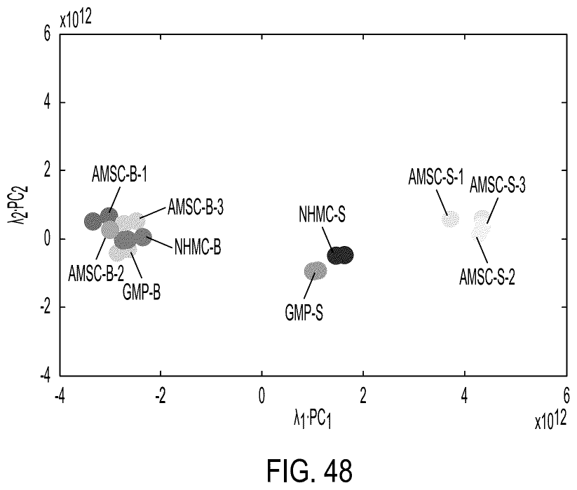

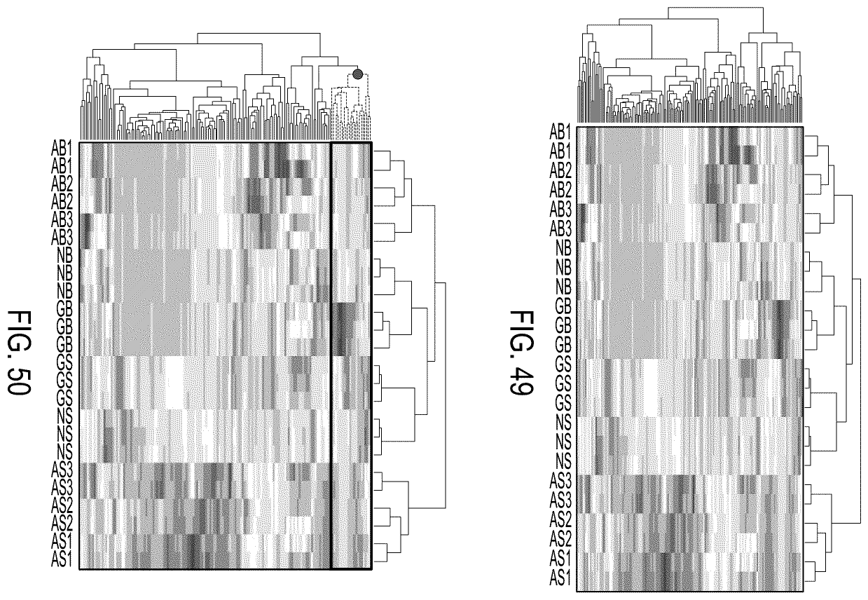

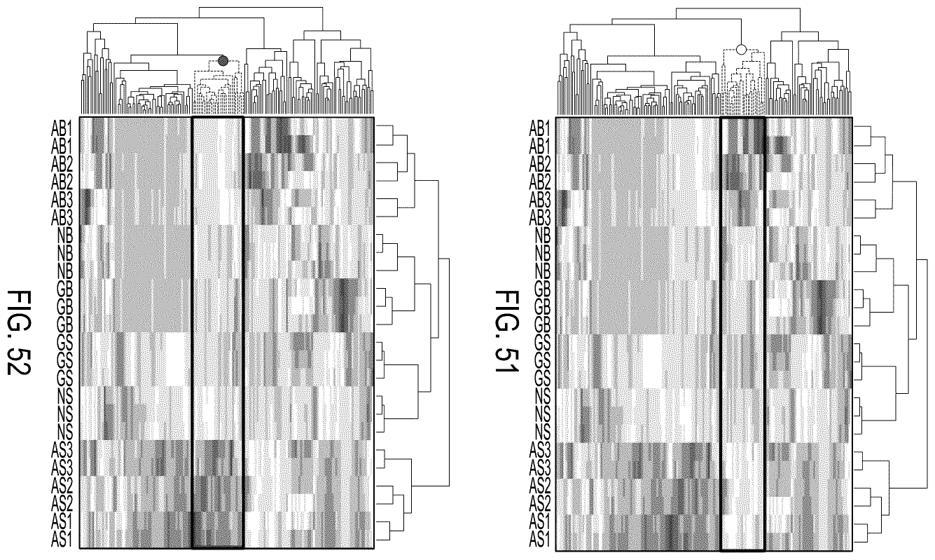

HMCs were also generated from a different pluripotent stem cell, i.e., GMP1 cells. Principal component analysis of transcriptomes of GMP1-HMC was also performed and compared with HMC derived from N-line cells (N-HMCs) and AD-MSCs under both basal and stimulated conditions (FIG. 48).

Hierarchical clustering analysis showed that GMP1-HMCshad similar profiles to the N-HMCs (FIG. 49). As shown in FIG. 50, genes in this cluster were up-regulated in N-HMCsand GMP1-HMCs (both basal and INFN gamma-stimulated) as compared to AD-MSCs. The genes included: TMSB4X, ACTG1, GSTP1, KRT18, IGFBP5, NPY, KRT8, PRDX6, MDK, DKK3, UCHL1, TUBB3, HN1, PTMA, HSP90AB1, HMGA1, HSPA8, TAGLN2, ANKRD1, PFN1, CYBA, and UBB. Differential expression of these genes between N-HMC, GMP1-HMC, and adipose tissue-derived MSC lines was consistent across biological and technical replicates according to the hierarchical clustering map.

Functional annotation of biological pathways enriched in the cluster on FIG. 50 was performed using Reactome (https://reactome.org/). The top pathway enriched by the corresponding genes was associated with axon guidance. Other significantly enriched pathways included cellular stress response and developmental biology.

As shown in FIG. 51, genes in these cluster were down-regulated in N-HMCs and GMP1-HMCs in basal condition as compared to AD-MSCs. The genes included: SERPINE1, S100A6, CD59, POSTN, VIM, MFAP5, ISLR, THBS1, COL6A3, TIMP3, ELN, ANXA2, COL1A1, BCYRN1, CCDC80, COL6A1, COL6A2, BGN, COL1A2, COL3A1, TGFB1, CRLF1, COMP, and GREM1. Differential expression of these genes between N-HMC, GMP1-HMC, and AD-MSC lines was consistent across biological and technical replicates according to the hierarchical clustering map.

As shown in FIG. 52, genes in these cluster were down-regulated in N-HMCs and GMP1-HMCs in INFN gamma-stimulated condition as compared to AD-MSCs. The genes included: MT1X, MT1G, TMSB10, CCL8, INHBA, CTSB, SERPINB2, ADM, APOL1, FTH1, CCL2, CCL5, CSF1, IL1B, IGFBP3, P4HB, DCN, FSTL1, ANXA5, LOX, CD63, CTSZ, FN1, LGALS1, LDHA, RCN3, MMP2, and TIMP1. Differential expression of these genes between N-HMC, GMP1-HMC, and AD-MSC lines was consistent across biological and technical replicates according to the hierarchical clustering map.

Functional annotation of biological pathways enriched in the cluster on FIGS. 51 and 52 was performed using Reactome (https://reactome.org/). The top pathways enriched by the corresponding genes were associated with extracellular matrix organization in general as well as collagen biosynthesis.

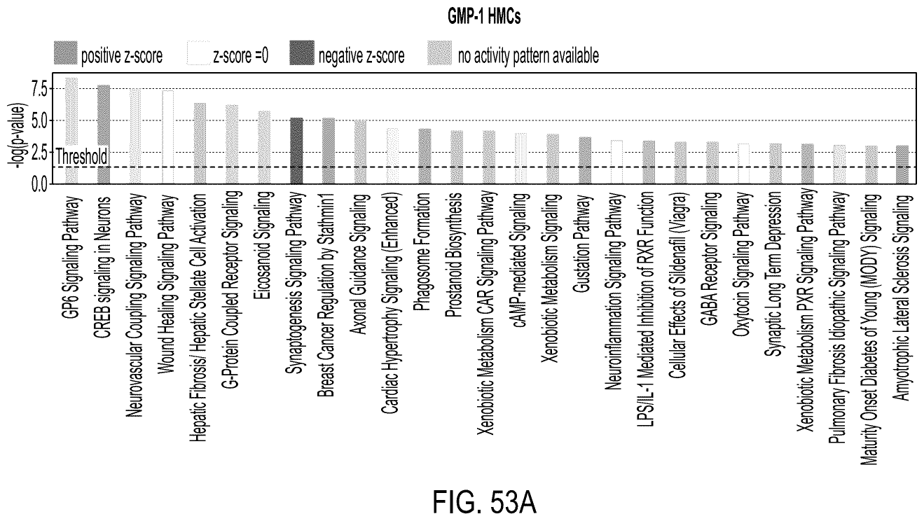

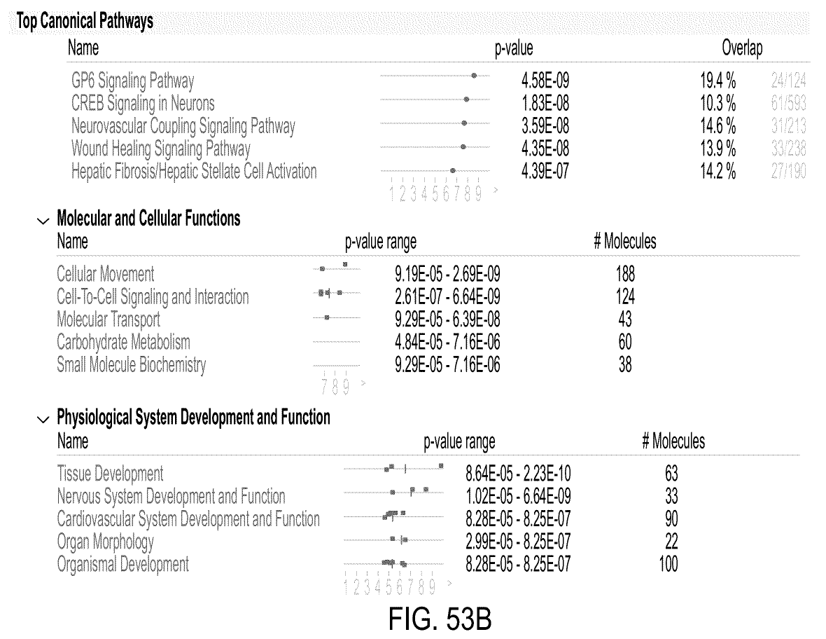

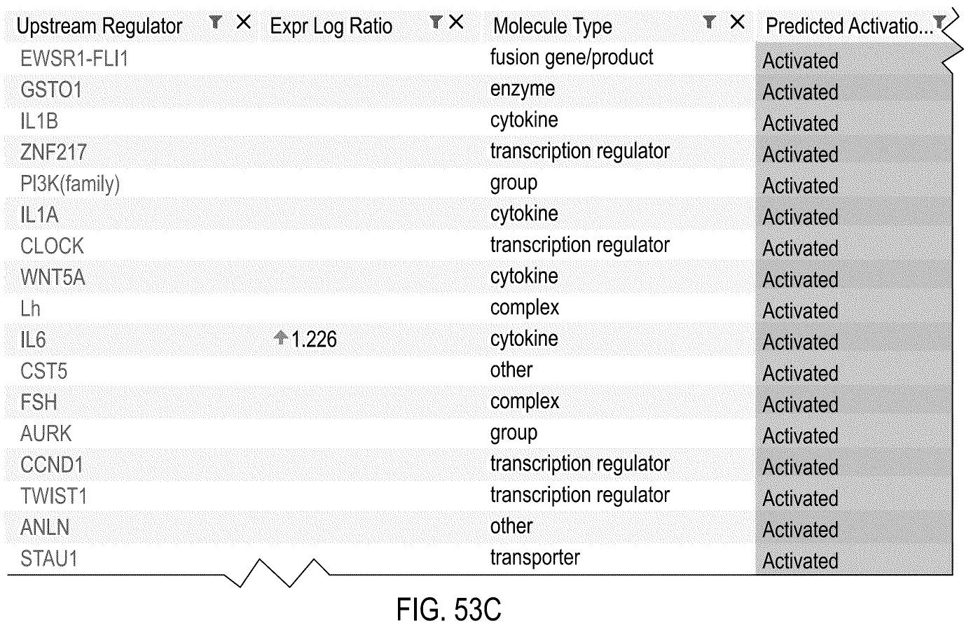

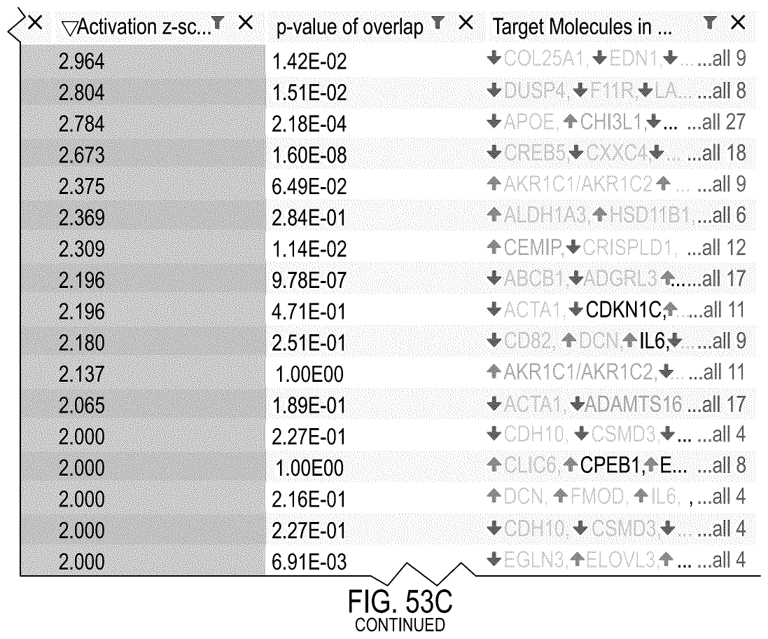

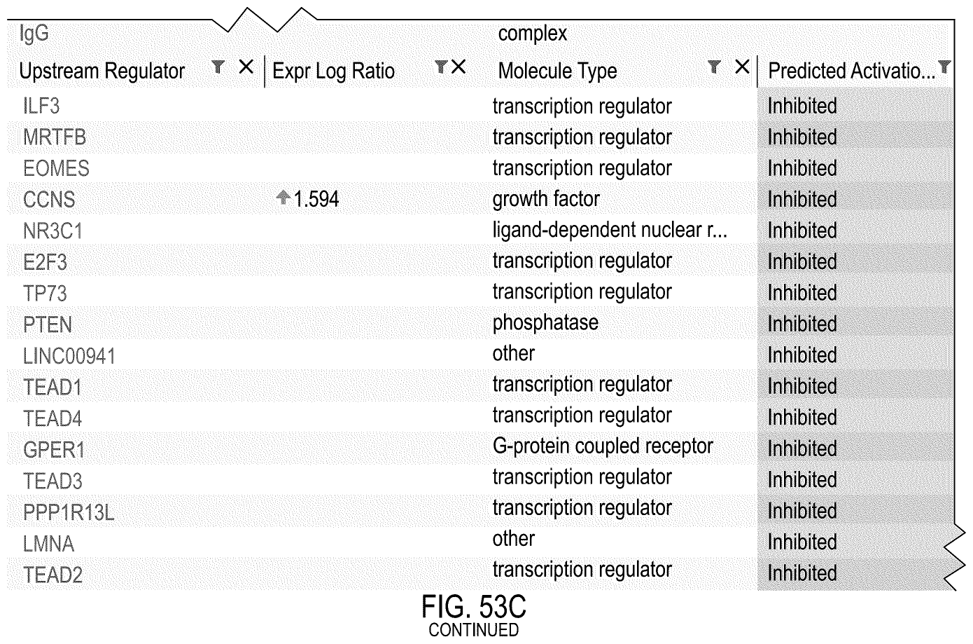

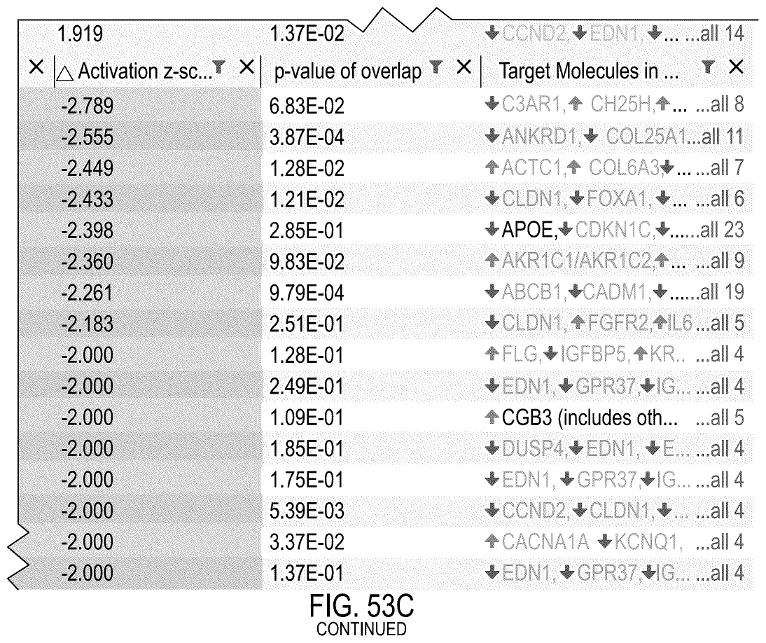

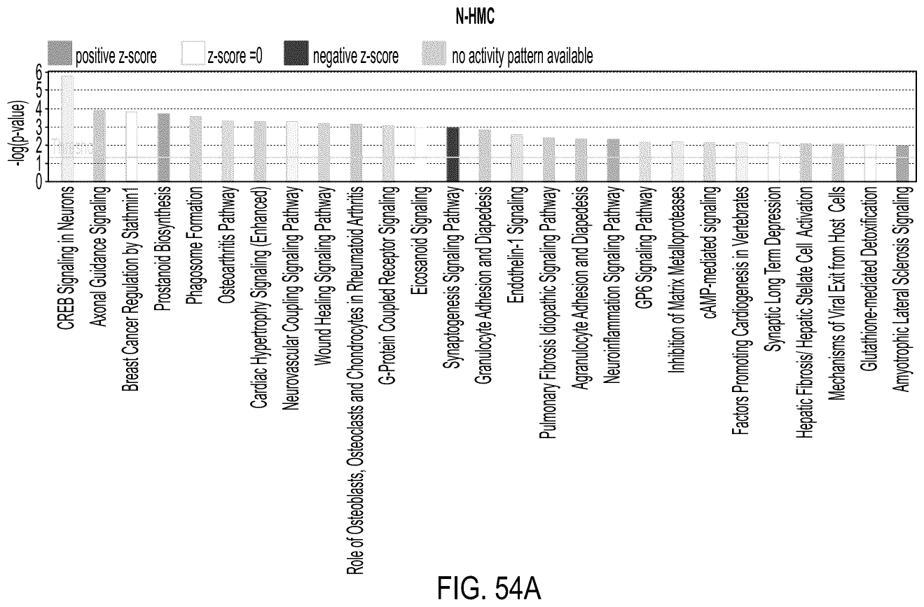

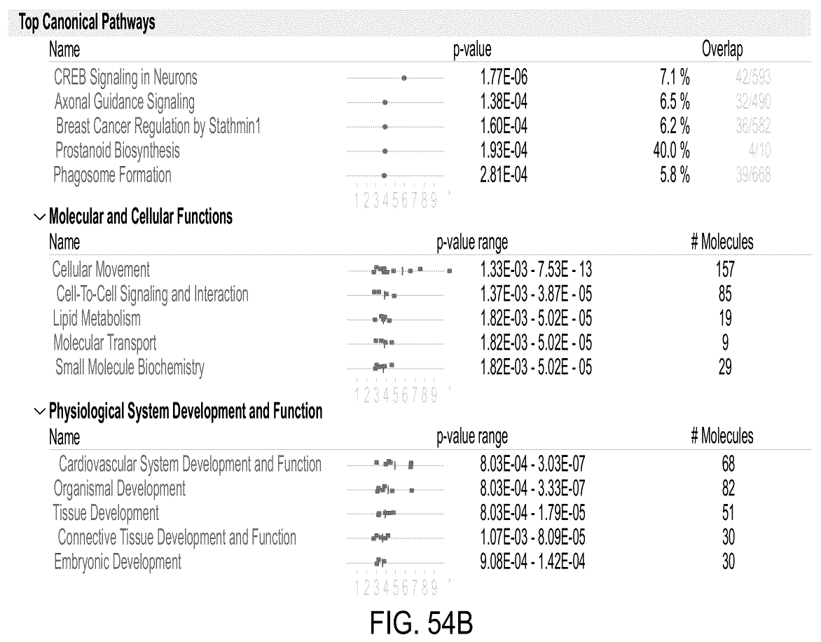

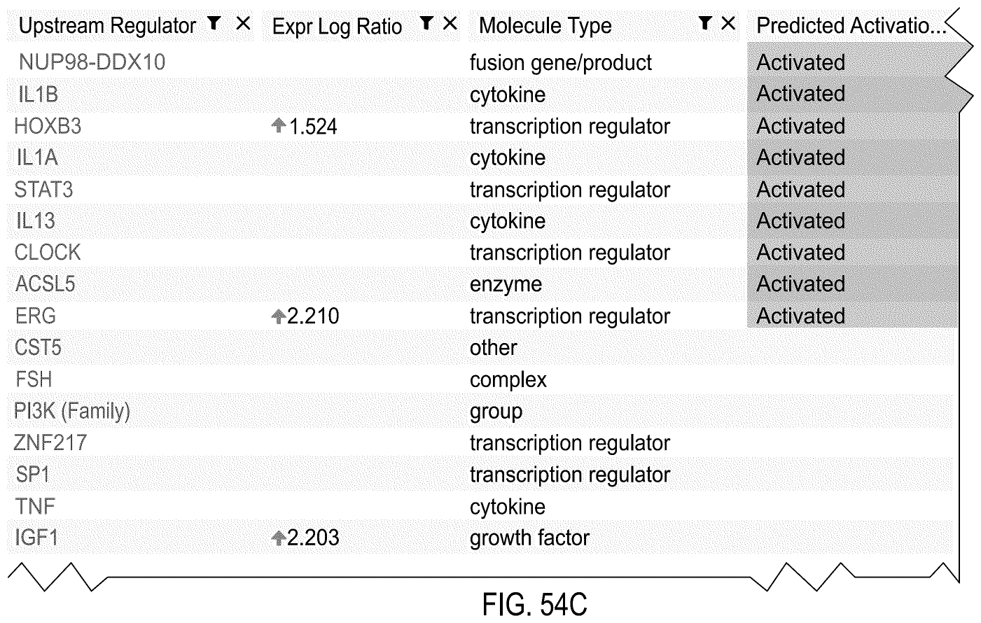

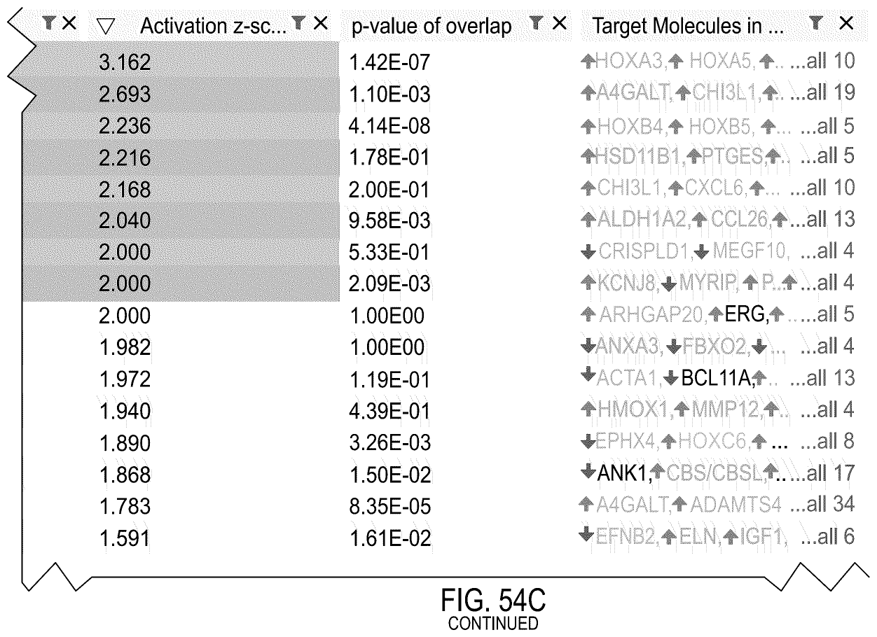

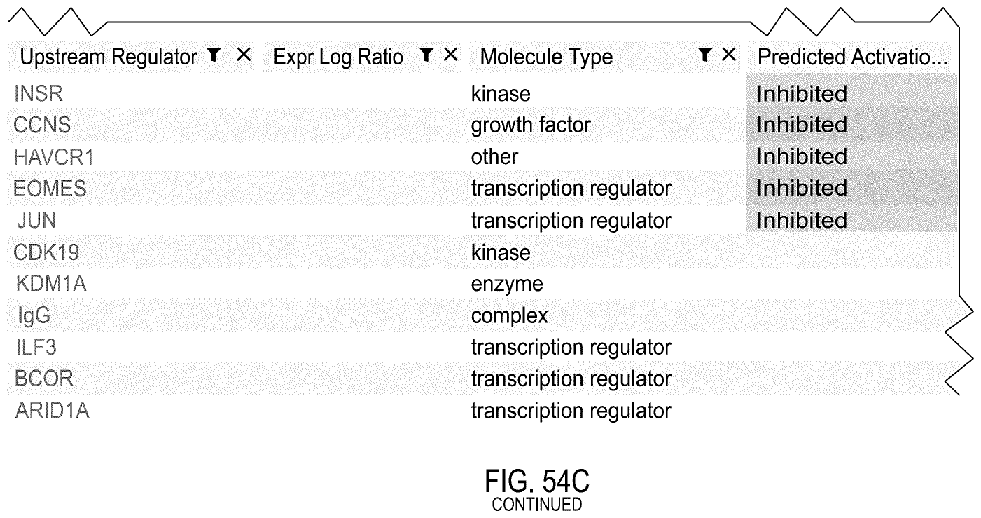

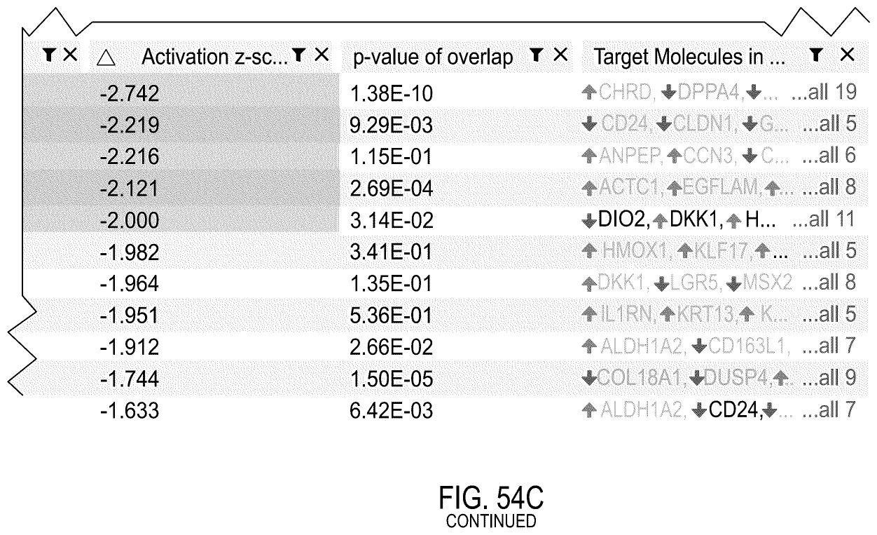

Similarly, canonical pathway enrichment of differential gene expression signature between N-HMCs, GMP1-HMCs, and AD-MSCs shows noticeable HMC-specific up-regulation of several pathways (denoted by red arrows) involved in the development of neuronal lineage including axon guidance, CREB signaling in neurons, synaptogenesis signaling etc. (FIGS. 53A-C and 54A-C). Thus, N-HMC and GMP1-HMCs shared a similar profile and both showed axon guidance enrichment.

Accordingly, it is concluded that the HMCs of the presently disclosed subject matter are distinct from AD-MSCs. Specifically, the MSCs of the presently disclosed subject matter have a distinct expression profile when compared to AD-MSCs, and may confer neuroprotective effects, provide neurotrophic factors, i.e., factors involved in supporting neuronal survival, growth, health and recovery.

HMC Vs. Bone Marrow Derived MSC

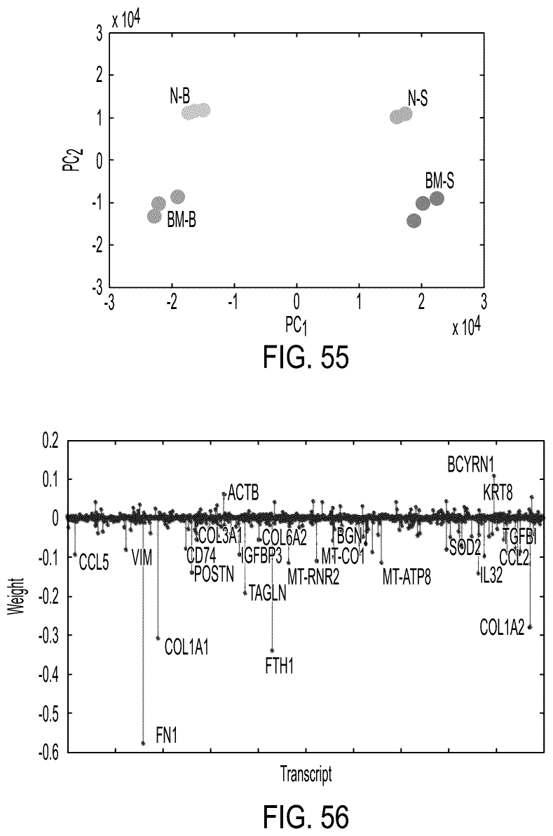

Principal component analysis of transcriptomes of HMCs (obtained from N-cell line) and BM-MSCs shows that HMCs are distinct from the latter in both basal and INFN-gamma stimulated state. The 1^{st} principal component largely describes the effect of stimulation with gamma interferon, while the 2^{nd} principal component describes the difference between HMCs and BM-MSCs (FIG. 55).

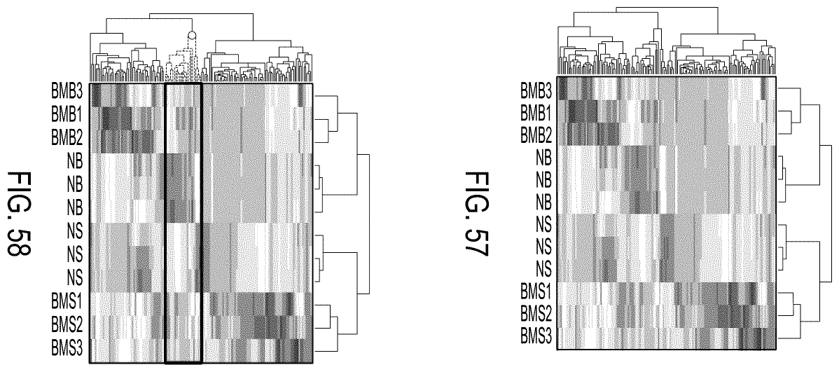

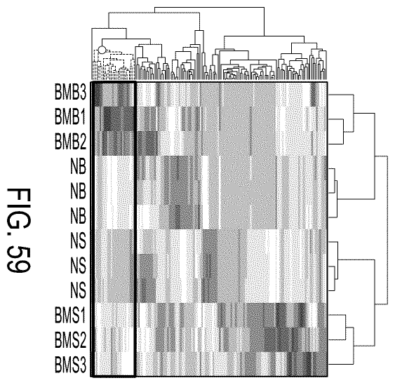

Weights of different genes contributing to the 2^{nd} principal component which determines the variance between HMCs and BM-MSCs. Of a particular note is down-regulation of collagen genes (COL1A1, COL1A2, COL3A1, COL6A2 etc.), mitochondrial function genes and TGF Beta 1 (one of the main factors promoting angiogenesis) in HMCs as compared to BM-MSCs demonstrating a certain degree of immaturity of HMCs as compared to the latter (FIG. 56).

Hierarchical clustering demonstrates similarity between biological replicate samples of the same type as well as clear difference between HMCs and BM-MSCs, in both basal cell states and cell states stimulated with gamma interferon (FIG. 57).

Genes in this cluster were up-regulated in HMCs (both basal and INFN gamma-stimulated) as compared to BM-MSCs (FIG. 58). The genes included: PPIA, NPM1, HNRNPA1, IGFBP5, KRT19, KRT18, GSTP1, TUBB, TUBA1B, KRT8, HN1, PTMA, TUBA1C, HSPA8, HMGA1, CFL1, MYL6, ACTB, UCHL1, TAGLN2, MDK, GREM1, MMP1, and CTSC. Differential expression of these genes between HMCs and BM-MSCs was consistent across biological and technical replicates according to the hierarchical clustering map.

Functional annotation of biological pathways enriched in the cluster on FIG. 58 was performed using Reactome (https://reactome.org/). Among the top pathways enriched by the corresponding genes there is axon guidance. Other significantly enriched pathways included cellular stress response and developmental biology (related to the relative immaturity of HMCs).

Genes in this cluster were down-regulated in HMCs (both basal and INFN gamma-stimulated) as compared to BM-MSCs (FIG. 59). The genes included: ANXA2, TPT1, VIM, COL6A1, BGN, COL6A2, CTGF, TIMP3, ACTA2, COL3A1, SPARC, ITGB1, SERPINH1, TPM2, TGFBI, COL1A1, TPM1, COL6A3, TPM4, SERPINE2, CALD1, COL1A2, TAGLN, MYL9, MT-RNR2, POSTN. Differential expression of these genes between HMCs and BM-MSCs was consistent across biological and technical replicates according to the hierarchical clustering map.

Functional annotation of biological pathways enriched in the cluster on FIG. 59 was performed using Reactome (https://reactome.org/). The top pathways enriched by the corresponding genes were associated with collagen biosynthesis/assembly (demonstrating similarities between BM-MSCs and AD-MSCs).

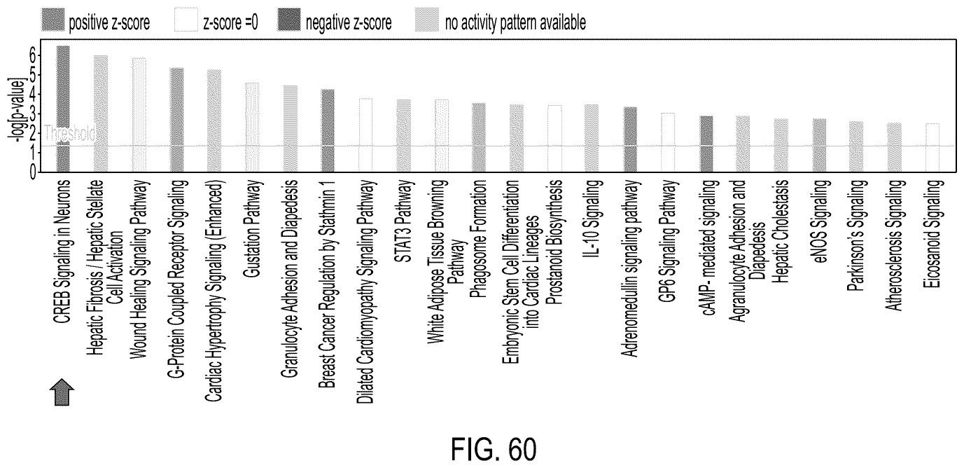

Canonical pathway enrichment of differential gene expression signature between HMCs and BM-MSCs again shows an HMC-specific up-regulation of pathways involved in the development of neuronal lineage such as CREB signaling in neurons (FIG. 60).

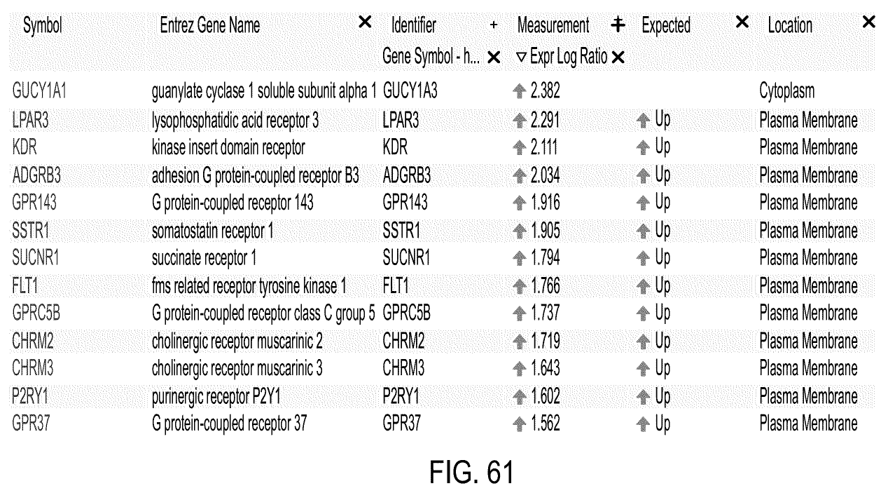

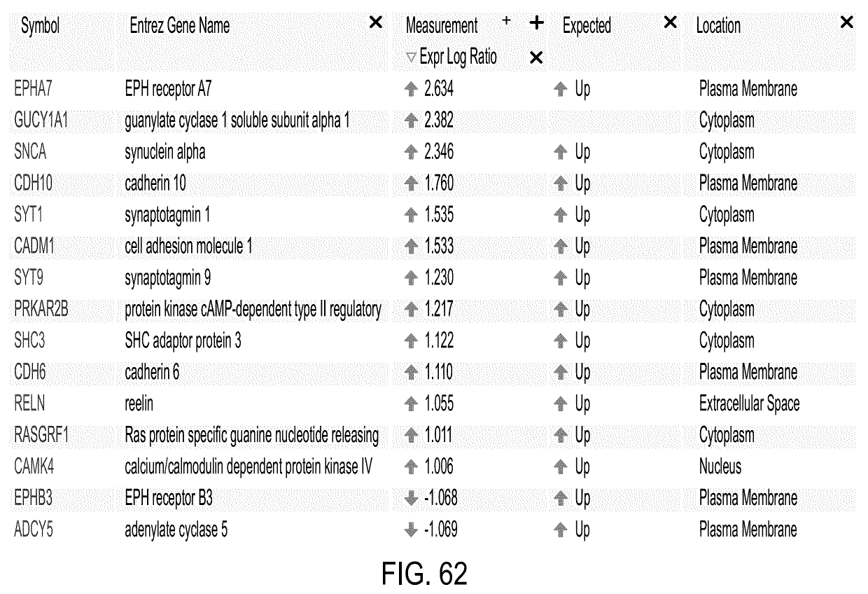

FIG. 61 depicts the top 15 most strongly differentially expressed genes contributing to activation of neuronal CREB signaling in HMCs as compared to BM-MSCs. FIG. 62. depicts the top 15 most strongly upregulated genes contributing to activation of synaptogenesis signaling in HMCs as compared to BM-MSCs.

Accordingly, it is concluded that, the HMCs of the presently disclosed subject matter are distinct from BM-MSCs. Specifically, the HMCs of the presently disclosed subject matter have a distinct expression profile, and provide neuroprotective effects when compared to BM-MSCs.

TABLE 7
Genes more highly expressed in HMCs compared with AD-MSCs

| | | Fold | Log Fold | |
|---|---|---|---|---|
| Gene name | Description | Change | Change | p-Adj |
| LOC400655 | uncharacterized LOC400655 | 298.97 | 8.2239 | 2.40E−39 |
| BAI3 | adhesion G protein-coupled receptor | 234.52 | 7.8735 | 2.09E−44 |
| | B3 | | | |
| SHISA2 | shisa family member 2 | 197.93 | 7.6288 | 9.51E−133 |
| CYYR1 | cysteine/tyrosine-rich 1 | 190.66 | 7.5749 | 1.02E−30 |
| PAX7 | paired box 7 | 181.34 | 7.5025 | 4.73E−29 |
| SYT14 | synaptotagmin XIV | 181.02 | 7.5000 | 1.15E−39 |
| ELAVL2 | ELAV like neuron-specific RNA | 158.73 | 7.3105 | 4.28E−80 |
| | binding protein 2 | | | |
| DCC | DCC netrin 1 receptor | 157.22 | 7.2966 | 3.12E−59 |
| WDR72 | WD repeat domain 72 | 156.71 | 7.2920 | 6.07E−32 |
| TMEM40 | transmembrane protein 40 | 137.10 | 7.0991 | 3.16E−33 |
| TTTY15 | testis-specific transcript_Y-linked | 123.88 | 6.9528 | 1.15E−131 |
| | 15 (non-protein coding) | | | |
| HRH2 | histamine receptor H2 | 112.31 | 6.8114 | 2.57E−22 |
| CA8 | carbonic anhydrase VIII | 103.90 | 6.6990 | 3.78E−28 |
| TFAP2A | transcription factor AP-2 alpha | 101.18 | 6.6608 | 1.12E−48 |
| | (activating enhancer binding protein | | | |
| | 2 alpha) | | | |
| ZDHHC8P1 | zinc finger_DHHC-type containing | 97.79 | 6.6115 | 2.23E−75 |
| | 8 pseudogene 1 | | | |
| DENND2A | DENN/MADD domain containing | 83.16 | 6.3778 | 2.98E−72 |
| | 2A | | | |
| HOPX | HOP homeobox | 78.67 | 6.2978 | 1.14E−29 |
| SYT13 | synaptotagmin XIII | 72.13 | 6.1726 | 1.68E−29 |
| KC6 | keratoconus gene 6 | 71.51 | 6.1602 | 1.97E−21 |
| KDM5D | lysine (K)-specific demethylase 5D | 68.12 | 6.0899 | 1.32E−90 |
| UTY | ubiquitously transcribed | 67.39 | 6.0744 | 1.17E−124 |
| | tetratricopeptide repeat containing_{—} | | | |
| | Y-linked | | | |
| SULT1C4 | sulfotransferase family_cytosolic_{—} | 67.28 | 6.0721 | 1.62E−18 |
| | 1C_member 4 | | | |
| MAB21L2 | mab-21-like 2 (C. elegans) | 64.72 | 6.0161 | 1.10E−13 |
| ZIC2 | Zic family member 2 | 64.55 | 6.0124 | 5.67E−45 |
| LOC644919 | uncharacterized LOC644919 | 63.85 | 5.9965 | 5.51E−22 |
| USP9Y | ubiquitin specific peptidase 9_Y- | 62.40 | 5.9634 | 5.67E−57 |
| | linked | | | |
| MSX2 | msh homeobox 2 | 60.69 | 5.9233 | 1.33E−41 |
| GATA3 | GATA binding protein 3 | 59.60 | 5.8973 | 2.70E−62 |
| RIPK4 | receptor-interacting serine-threonine | 59.03 | 5.8833 | 1.18E−61 |
| | kinase 4 | | | |
| PKIB | protein kinase (cAMP-dependent_{—} | 58.55 | 5.8717 | 1.61E−22 |
| | catalytic) inhibitor beta | | | |
| GAL3ST3 | galactose-3-O-sulfotransferase 3 | 58.19 | 5.8627 | 1.39E−21 |
| CASC9 | cancer susceptibility candidate 9 | 56.08 | 5.8095 | 1.34E−24 |
| | (non-protein coding) | | | |
| TGFB2 | transforming growth factor_beta 2 | 53.17 | 5.7324 | 2.52E−45 |
| L1CAM | L1 cell adhesion molecule | 53.09 | 5.7305 | 2.84E−117 |
| TXLNGY | taxilin gamma pseudogene_Y- | 50.60 | 5.6610 | 3.79E−98 |
| | linked | | | |
| EIF1AY | eukaryotic translation initiation | 50.17 | 5.6487 | 3.91E−55 |
| | factor 1A_Y-linked | | | |
| RPS4Y1 | ribosomal protein S4_Y-linked 1 | 48.25 | 5.5925 | 6.04E−33 |
| PCDHA2 | protocadherin alpha 2 | 47.12 | 5.5582 | 5.20E−33 |
| LINC00648 | long intergenic non-protein coding | 46.20 | 5.5298 | 2.25E−16 |
| | RNA 648 | | | |
| SNRPN | small nuclear ribonucleoprotein | 45.49 | 5.5075 | 2.85E−23 |
| | polypeptide N | | | |
| PRKY | protein kinase_Y-linked_{—} | 44.67 | 5.4813 | 5.09E−58 |
| | pseudogene | | | |
| TTTY14 | testis-specific transcript_Y-linked | 44.52 | 5.4764 | 3.51E−12 |
| | 14 (non-protein coding) | | | |
| PCDHB5 | protocadherin beta 5 | 43.99 | 5.4592 | 7.71E−125 |
| SDK2 | sidekick cell adhesion molecule 2 | 43.14 | 5.4310 | 3.20E−46 |
| CDH3 | cadherin 3_type 1_P-cadherin | 43.08 | 5.4289 | 7.17E−39 |
| | (placental) | | | |
| FZD10-AS1 | FZD10 antisense RNA 1 (head to | 42.97 | 5.4251 | 2.77E−12 |
| | head) | | | |
| CD24 | CD24 molecule | 41.69 | 5.3818 | 2.94E−211 |
| C7orf69 | chromosome 7 open reading frame | 40.57 | 5.3422 | 1.20E−33 |
| | 69 | | | |
| NETO1 | neuropilin (NRP) and tolloid (TLL)- | 40.16 | 5.3277 | 1.29E−66 |
| | like 1 | | | |
| SOX11 | SRY (sex determining region Y)- | 40.07 | 5.3244 | 5.46E−13 |
| | box 11 | | | |
| SLC7A2 | solute carrier family 7 (cationic | 39.40 | 5.3002 | 3.79E−13 |
| | amino acid transporter_y + system)_{—} | | | |
| | member 2 | | | |
| NLGN4X | neuroligin 4_X-linked | 38.78 | 5.2773 | 2.05E−11 |
| MDFI | MyoD family inhibitor | 38.75 | 5.2762 | 1.58E−226 |
| GABRB1 | gamma-aminobutyric acid (GABA) | 38.15 | 5.2535 | 1.20E−15 |
| | A receptor_beta 1 | | | |
| LOC100507600 | uncharacterized LOC100507600 | 36.76 | 5.1999 | 1.30E−19 |
| DDX3Y | DEAD (Asp-Glu-Ala-Asp) box | 36.52 | 5.1908 | 1.14E−21 |
| | helicase 3_Y-linked | | | |
| IGF2-AS | IGF2 antisense RNA | 35.47 | 5.1486 | 1.14E−10 |
| GPRC5C | G protein-coupled receptor_class | 35.30 | 5.1415 | 3.14E−44 |
| | C_group 5_member C | | | |
| MSLN | mesothelin | 35.29 | 5.1412 | 1.09E−10 |
| LPAR4 | lysophosphatidic acid receptor 4 | 35.24 | 5.1392 | 2.90E−22 |
| EFNA1 | ephrin-A1 | 34.82 | 5.1217 | 2.55E−31 |
| MUM1L1 | melanoma associated antigen | 33.17 | 5.0516 | 1.14E−10 |
| | (mutated) 1-like 1 | | | |
| C7 | complement component 7 | 32.85 | 5.0377 | 1.03E−09 |
| NLGN4Y | neuroligin 4_Y-linked | 32.76 | 5.0340 | 1.43E−14 |
| PCDHA12 | protocadherin alpha 12 | 32.56 | 5.0249 | 1.18E−11 |
| TFAP2A-AS1 | TFAP2A antisense RNA 1 | 32.47 | 5.0211 | 1.24E−17 |
| CDH18 | cadherin 18_type 2 | 32.36 | 5.0160 | 6.15E−13 |
| DPY19L2P1 | DPY19L2 pseudogene 1 | 31.57 | 4.9804 | 2.43E−15 |
| GABRA3 | gamma-aminobutyric acid (GABA) | 30.86 | 4.9475 | 4.71E−18 |
| | A receptor_alpha 3 | | | |
| CLDN1 | claudin 1 | 30.81 | 4.9454 | 8.36E−18 |
| CYP27C1 | cytochrome P450_family 27_{—} | 30.78 | 4.9439 | 1.65E−17 |
| | subfamily C_polypeptide 1 | | | |
| IGSF9B | immunoglobulin superfamily_{—} | 30.52 | 4.9316 | 8.19E−25 |
| | member 9B | | | |
| C5orf46 | chromosome 5 open reading frame | 30.22 | 4.9175 | 1.02E−09 |
| | 46 | | | |
| C1orf94 | chromosome 1 open reading frame | 30.16 | 4.9148 | 1.70E−10 |
| | 94 | | | |
| NEDD4L | neural precursor cell expressed_{—} | 29.64 | 4.8895 | 4.58E−81 |
| | developmentally down-regulated 4- | | | |
| | like_E3 ubiquitin protein ligase | | | |
| MLC1 | megalencephalic | 29.14 | 4.8650 | 2.64E−10 |
| | leukoencephalopathy with | | | |
| | subcortical cysts 1 | | | |
| DLX1 | distal-less homeobox 1 | 29.04 | 4.8601 | 3.14E−116 |
| PAX3 | paired box 3 | 28.76 | 4.8457 | 6.05E−156 |
| PCDHAC2 | protocadherin alpha subfamily C_2 | 28.62 | 4.8388 | 2.47E−22 |
| MAGEL2 | melanoma antigen family L2 | 28.59 | 4.8374 | 4.18E−21 |
| PLCH2 | phospholipase C_eta 2 | 28.36 | 4.8256 | 3.33E−11 |
| NR0B1 | nuclear receptor subfamily 0_group | 28.35 | 4.8253 | 3.16E−17 |
| | B_member 1 | | | |
| CCNJL | cyclin J-like | 28.31 | 4.8232 | 5.67E−16 |
| SORCS1 | sortilin-related VPS10 domain | 27.98 | 4.8064 | 6.23E−10 |
| | containing receptor 1 | | | |
| VANGL2 | VANGL planar cell polarity protein | 27.96 | 4.8054 | 3.88E−14 |
| | 2 | | | |
| SALL1 | spalt-like transcription factor 1 | 27.92 | 4.8035 | 1.31E−18 |
| LOC102467080 | uncharacterized LOC102467080 | 27.08 | 4.7594 | 9.43E−11 |
| CRISPLD1 | cysteine-rich secretory protein | 26.77 | 4.7424 | 1.83E−16 |
| | LCCL domain containing 1 | | | |
| TMEM132D | transmembrane protein 132D | 26.14 | 4.7082 | 9.35E−11 |
| PRKCQ-AS1 | PRKCQ antisense RNA 1 | 25.48 | 4.6711 | 2.53E−17 |
| CACNG4 | calcium channel_voltage- | 25.36 | 4.6644 | 1.76E−08 |
| | dependent_gamma subunit 4 | | | |
| KIAA1211 | KIAA1211 | 25.20 | 4.6553 | 2.27E−31 |
| ANXA3 | annexin A3 | 25.16 | 4.6532 | 2.77E−46 |
| NMNAT3 | nicotinamide nucleotide | 25.10 | 4.6493 | 3.46E−09 |
| | adenylyltransferase 3 | | | |
| SLAMF7 | SLAM family member 7 | 24.98 | 4.6427 | 8.99E−13 |
| GPR20 | G protein-coupled receptor 20 | 24.72 | 4.6275 | 9.88E−11 |
| OLFML2A | olfactomedin-like 2A | 24.60 | 4.6206 | 4.62E−40 |
| IP6K3 | inositol hexakisphosphate kinase 3 | 24.54 | 4.6172 | 1.08E−10 |
| LMX1B | LIM homeobox transcription factor | 24.37 | 4.6070 | 7.25E−15 |
| | 1_beta | | | |
| IGF2 | insulin-like growth factor 2 | 24.24 | 4.5992 | 3.10E−08 |
| KCNK3 | potassium channel_two pore | 24.24 | 4.5991 | 3.99E−08 |
| | domain subfamily K_member 3 | | | |
| ZFY | zinc finger protein_Y-linked | 23.97 | 4.5833 | 1.42E−09 |
| CLSTN2 | calsyntenin 2 | 23.89 | 4.5781 | 6.01E−11 |
| GNAZ | guanine nucleotide binding protein | 23.80 | 4.5728 | 1.11E−90 |
| | (G protein)_alpha z polypeptide | | | |
| GCNT2 | glucosaminyl (N-acetyl) transferase | 23.61 | 4.5616 | 2.98E−28 |
| | 2_I-branching enzyme (I blood | | | |
| | group) | | | |
| PCDHB15 | protocadherin beta 15 | 23.53 | 4.5564 | 2.81E−46 |
| PCDHA10 | protocadherin alpha 10 | 23.47 | 4.5527 | 3.83E−16 |
| C11orf88 | chromosome 11 open reading frame | 23.47 | 4.5527 | 3.83E−11 |
| | 88 | | | |
| MGAT5B | mannosyl (alpha-1_6-)-glycoprotein | 23.21 | 4.5366 | 6.99E−73 |
| | beta-1_6-N-acetyl- | | | |
| | glucosaminyltransferase_isozyme B | | | |
| OVCH2 | ovochymase 2 (gene/pseudogene) | 23.17 | 4.5344 | 2.35E−11 |
| ATRNL1 | attractin-like 1 | 23.05 | 4.5266 | 8.08E−18 |
| TEX15 | testis expressed 15 | 22.84 | 4.5138 | 2.28E−12 |
| SHROOM2 | shroom family member 2 | 22.83 | 4.5131 | 4.36E−10 |
| ECEL1P2 | endothelin converting enzyme-like | 22.60 | 4.4985 | 6.48E−10 |
| | 1_pseudogene 2 | | | |
| SDK1 | sidekick cell adhesion molecule 1 | 22.28 | 4.4780 | 1.76E−24 |
| EPHB2 | EPH receptor B2 | 22.27 | 4.4773 | 2.63E−18 |
| MIR4697HG | MIR4697 host gene | 22.12 | 4.4675 | 2.04E−17 |
| ABCA13 | ATP-binding cassette_sub-family A | 21.72 | 4.4407 | 2.93E−17 |
| | (ABC1)_member 13 | | | |
| C21orf88 | B3GALT5 antisense RNA 1 | 21.46 | 4.4238 | 1.23E−09 |
| LIN28B | lin-28 homolog B (C. elegans) | 21.46 | 4.4233 | 1.04E−19 |
| LINC01158 | long intergenic non-protein coding | 21.14 | 4.4018 | 1.41E−08 |
| | RNA 1158 | | | |
| RASGRF1 | Ras protein-specific guanine | 21.12 | 4.4004 | 1.98E−13 |
| | nucleotide-releasing factor 1 | | | |
| GRIA1 | glutamate receptor_ionotropic_{—} | 20.59 | 4.3639 | 6.29E−25 |
| | AMPA 1 | | | |
| LINC00491 | long intergenic non-protein coding | 20.56 | 4.3619 | 1.12E−08 |
| | RNA 491 | | | |
| PCDHB2 | protocadherin beta 2 | 20.19 | 4.3355 | 1.69E−71 |
| ZNF853 | zinc finger protein 853 | 19.98 | 4.3202 | 6.14E−46 |
| SERPINA5 | serpin peptidase inhibitor_clade A | 19.89 | 4.3138 | 1.54E−10 |
| | (alpha-1 antiproteinase_{—} | | | |
| | antitrypsin)_member 5 | | | |
| CA3 | carbonic anhydrase III | 19.47 | 4.2832 | 1.71E−07 |
| PLEKHA6 | pleckstrin homology domain | 19.34 | 4.2734 | 1.17E−22 |
| | containing_family A member 6 | | | |
| LOC283299 | uncharacterized LOC283299 | 19.22 | 4.2642 | 2.33E−08 |
| NRK | Nik related kinase | 18.95 | 4.2444 | 2.17E−47 |
| LINC00460 | long intergenic non-protein coding | 18.91 | 4.2414 | 3.71E−08 |
| | RNA 460 | | | |
| MYO5C | myosin VC | 18.88 | 4.2390 | 1.38E−12 |
| ANK1 | ankyrin 1_erythrocytic | 18.61 | 4.2182 | 1.01E−25 |
| NIPAL4 | NIPA-like domain containing 4 | 18.46 | 4.2066 | 8.70E−10 |
| SAMD5 | sterile alpha motif domain | 18.35 | 4.1981 | 6.95E−07 |
| | containing 5 | | | |
| SOWAHD | sosondowah ankyrin repeat domain | 18.22 | 4.1874 | 3.96E−18 |
| | family member D | | | |
| CIDEA | cell death-inducing DFFA-like | 18.04 | 4.1732 | 1.37E−06 |
| | effector a | | | |
| SHF | Src homology 2 domain containing | 17.93 | 4.1643 | 9.93E−91 |
| | F | | | |
| GABRQ | gamma-aminobutyric acid (GABA) | 17.93 | 4.1639 | 8.84E−09 |
| | A receptor theta | | | |
| NFE2L3 | nuclear factor_erythroid 2-like 3 | 17.87 | 4.1596 | 4.45E−50 |
| CRHBP | corticotropin releasing hormone | 17.49 | 4.1285 | 2.10E−08 |
| | binding protein | | | |
| SPTBN2 | spectrin_beta_non-erythrocytic 2 | 17.41 | 4.1219 | 3.91E−106 |
| INA | internexin neuronal intermediate | 17.37 | 4.1188 | 1.25E−22 |
| | filament protein_alpha | | | |
| VAX1 | ventral anterior homeobox 1 | 17.32 | 4.1144 | 8.06E−07 |
| CDKL2 | cyclin-dependent kinase-like 2 | 17.11 | 4.0971 | 2.86E−12 |
| | (CDC2-related kinase) | | | |
| GLIS1 | GLIS family zinc finger 1 | 17.08 | 4.0943 | 6.84E−149 |
| IRF6 | interferon regulatory factor 6 | 16.81 | 4.0711 | 7.61E−11 |
| POU3F3 | POU class 3 homeobox 3 | 16.77 | 4.0680 | 1.72E−10 |
| LOC339975 | uncharacterized LOC339975 | 16.72 | 4.0639 | 3.00E−08 |
| RASL10B | RAS-like_family_10_member B | 16.67 | 4.0590 | 1.14E−52 |
| KLHL4 | kelch-like family member 4 | 16.57 | 4.0502 | 7.55E−23 |
| EN2 | engrailed homeobox 2 | 16.46 | 4.0405 | 3.11E−07 |
| FBXO2 | F-box protein 2 | 16.33 | 4.0291 | 9.42E−23 |
| CADM1 | cell adhesion molecule 1 | 16.17 | 4.0152 | 1.30E−11 |
| SIPA1L2 | signal-induced proliferation- | 16.14 | 4.0125 | 1.24E−23 |
| | associated 1 like 2 | | | |
| PAK3 | p21 protein (Cdc42/Rac)-activated | 16.08 | 4.0071 | 3.73E−38 |
| | kinase 3 | | | |
| EPHA5-AS1 | EPHA5 antisense RNA 1 | 15.99 | 3.9993 | 2.38E−06 |
| OPRD1 | opioid receptor_delta 1 | 15.91 | 3.9915 | 6.44E−06 |
| NIPAL1 | NIPA-like domain containing 1 | 15.83 | 3.9846 | 1.07E−09 |
| SRSF12 | serine/arginine-rich splicing factor | 15.68 | 3.9709 | 2.27E−10 |
| | 12 | | | |
| NNAT | neuronatin | 15.59 | 3.9623 | 2.99E−19 |
| FAM69B | family with sequence similarity 69_{—} | 15.49 | 3.9532 | 1.53E−83 |
| | member B | | | |
| DUSP8 | dual specificity phosphatase 8 | 15.45 | 3.9493 | 7.39E−44 |
| MAMDC2-AS1 | MAMDC2 antisense RNA 1 | 15.38 | 3.9433 | 1.48E−08 |
| MEX3A | mex-3 RNA binding family member | 15.32 | 3.9375 | 9.15E−96 |
| | A | | | |
| PLEKHG4B | pleckstrin homology domain | 15.18 | 3.9241 | 2.31E−23 |
| | containing_family G (with RhoGef | | | |
| | domain) member 4B | | | |
| EYA1 | EYA transcriptional coactivator and | 15.07 | 3.9137 | 1.31E−09 |
| | phosphatase 1 | | | |
| TIE1 | tyrosine kinase with | 15.03 | 3.9096 | 1.41E−17 |
| | immunoglobulin-like and EGF-like | | | |
| | domains 1 | | | |
| ARSE | arylsulfatase E (chondrodysplasia | 14.84 | 3.8914 | 1.74E−36 |
| | punctata 1) | | | |
| FAM110D | family with sequence similarity | 14.73 | 3.8807 | 1.42E−17 |
| | 110_member D | | | |
| PLCXD3 | phosphatidylinositol-specific | 14.68 | 3.8759 | 1.26E−05 |
| | phospholipase C_X domain | | | |
| | containing 3 | | | |
| SLC44A5 | solute carrier family 44_member 5 | 14.68 | 3.8753 | 1.15E−06 |
| PCSK1N | proprotein convertase | 14.66 | 3.8737 | 1.93E−06 |
| | subtilisin/kexin type 1 inhibitor | | | |
| IL31RA | interleukin 31 receptor A | 14.62 | 3.8701 | 1.26E−08 |
| PCDHGB6 | protocadherin gamma subfamily B_{—} | 14.54 | 3.8620 | 5.59E−70 |
| | 6 | | | |
| WSCD1 | WSC domain containing 1 | 14.47 | 3.8555 | 6.83E−06 |
| KLHL23 | kelch-like family member 23 | 14.36 | 3.8442 | 8.90E−08 |
| KCNF1 | potassium channel_voltage gated | 14.35 | 3.8430 | 2.97E−06 |
| | modifier subfamily F_member 1 | | | |
| TFAP2C | transcription factor AP-2 gamma | 14.26 | 3.8339 | 9.30E−08 |
| | (activating enhancer binding protein | | | |
| | 2 gamma) | | | |
| CD163L1 | CD163 molecule-like 1 | 14.13 | 3.8202 | 9.04E−28 |
| RAMP1 | receptor (G protein-coupled) activity | 13.96 | 3.8033 | 1.04E−07 |
| | modifying protein 1 | | | |
| C10orf126 | chromosome 10 open reading frame | 13.61 | 3.7662 | 2.35E−05 |
| | 126 | | | |
| CPXM1 | carboxypeptidase X (M14 family)_{—} | 13.60 | 3.7657 | 1.95E−58 |
| | member 1 | | | |
| SPINK5 | serine peptidase inhibitor_Kazal | 13.56 | 3.7614 | 4.59E−06 |
| | type 5 | | | |
| NCRNA00185 | testis-specific transcript_Y-linked | 13.51 | 3.7558 | 2.44E−05 |
| | 14 (non-protein coding) | | | |
| JAKMIP2 | janus kinase and microtubule | 13.39 | 3.7428 | 7.95E−12 |
| | interacting protein 2 | | | |
| SLC7A14 | solute carrier family 7_member 14 | 13.38 | 3.7415 | 1.86E−30 |
| B4GALNT4 | beta-1_4-N-acetyl-galactosaminyl | 13.35 | 3.7385 | 2.93E−10 |
| | transferase 4 | | | |
| ETNK2 | ethanolamine kinase 2 | 13.22 | 3.7248 | 1.53E−135 |
| SH2D3C | SH2 domain containing 3C | 13.17 | 3.7196 | 8.57E−08 |
| MAP3K9 | mitogen-activated protein kinase | 12.93 | 3.6923 | 2.32E−23 |
| | kinase kinase 9 | | | |
| SHC2 | SHC (Src homology 2 domain | 12.84 | 3.6829 | 1.46E−08 |
| | containing) transforming protein 2 | | | |
| PTGER4 | prostaglandin E receptor 4 (subtype | 12.81 | 3.6794 | 1.54E−59 |
| | EP4) | | | |
| EPHA5 | EPH receptor A5 | 12.70 | 3.6673 | 1.03E−18 |
| LINC01012 | long intergenic non-protein coding | 12.64 | 3.6596 | 9.52E−06 |
| | RNA 1012 | | | |
| IL2RB | interleukin 2 receptor_beta | 12.64 | 3.6595 | 1.10E−07 |
| GATA3-AS1 | GATA3 antisense RNA 1 | 12.62 | 3.6581 | 2.67E−06 |
| RIMS2 | regulating synaptic membrane | 12.60 | 3.6551 | 2.86E−22 |
| | exocytosis 2 | | | |
| ADAMTS3 | ADAM metallopeptidase with | 12.60 | 3.6549 | 1.02E−64 |
| | thrombospondin type 1 motif_3 | | | |
| PIEZO2 | piezo-type mechanosensitive ion | 12.55 | 3.6495 | 2.89E−08 |
| | channel component 2 | | | |
| GLP2R | glucagon-like peptide 2 receptor | 12.46 | 3.6393 | 3.38E−06 |
| GPRC5D | G protein-coupled receptor_class | 12.45 | 3.6382 | 4.22E−06 |
| | C_group 5_member D | | | |
| GBX2 | gastrulation brain homeobox 2 | 12.44 | 3.6366 | 1.28E−07 |
| TMEM255A | transmembrane protein 255A | 12.34 | 3.6257 | 8.27E−14 |
| LOC100506314 | uncharacterized LOC100506314 | 12.33 | 3.6240 | 5.74E−19 |
| LHX8 | LIM homeobox 8 | 12.31 | 3.6221 | 4.39E−06 |
| NOMO3 | NODAL modulator 3 | 12.30 | 3.6210 | 8.51E−148 |
| LINC00858 | long intergenic non-protein coding | 12.25 | 3.6152 | 5.74E−05 |
| | RNA 858 | | | |
| C2CD4C | C2 calcium-dependent domain | 12.22 | 3.6110 | 4.68E−14 |
| | containing 4C | | | |
| COL4A6 | collagen_typeIV_alpha 6 | 12.20 | 3.6084 | 4.19E−05 |
| CD6 | CD6 molecule | 12.18 | 3.6059 | 8.90E−07 |
| EFNB2 | ephrin-B2 | 12.06 | 3.5922 | 1.23E−06 |
| FOXF1 | forkhead box F1 | 11.99 | 3.5840 | 9.88E−22 |
| B3GNT5 | UDP-GlcNAc:betaGal beta-1_3-N- | 11.97 | 3.5812 | 6.07E−128 |
| | acetylglucosaminyltransferase 5 | | | |
| LINC00470 | long intergenic non-protein coding | 11.89 | 3.5720 | 4.17E−07 |
| | RNA 470 | | | |
| ADARB2 | adenosine deaminase_RNA- | 11.83 | 3.5640 | 2.75E−05 |
| | specific_B2 (non-functional) | | | |
| IGFBP2 | insulin-like growth factor binding | 11.82 | 3.5635 | 5.62E−05 |
| | protein 2_36 kDa | | | |
| LRP1B | low density lipoprotein receptor- | 11.82 | 3.5626 | 5.67E−05 |
| | related protein 1B | | | |
| DUSP4 | dual specificity phosphatase 4 | 11.81 | 3.5624 | 2.18E−42 |
| TRHDE-AS1 | TRHDE antisense RNA 1 | 11.78 | 3.5588 | 1.62E−05 |
| TFAP2B | transcription factor AP-2 beta | 11.77 | 3.5565 | 1.86E−05 |
| | (activating enhancer binding protein | | | |
| | 2 beta) | | | |
| BIRC7 | baculoviral IAP repeat containing 7 | 11.72 | 3.5505 | 3.89E−05 |
| TMCC3 | transmembrane and coiled-coil | 11.70 | 3.5482 | 4.68E−07 |
| | domain family 3 | | | |
| LINC00649 | long intergenic non-protein coding | 11.69 | 3.5470 | 3.31E−20 |
| | RNA 649 | | | |
| GDF5 | growth differentiation factor 5 | 11.64 | 3.5409 | 3.92E−09 |
| BEND5 | BEN domain containing 5 | 11.55 | 3.5293 | 3.37E−09 |
| AFAP1L2 | actin filament associated protein 1- | 11.44 | 3.5157 | 1.02E−16 |
| | like 2 | | | |
| SALL2 | spalt-like transcription factor 2 | 11.40 | 3.5109 | 3.93E−27 |
| FZD10 | frizzled class receptor 10 | 11.35 | 3.5045 | 6.53E−05 |
| DPPA4 | developmental pluripotency | 11.30 | 3.4989 | 1.23E−04 |
| | associated 4 | | | |
| MECOM | MDS1 and EVI1 complex locus | 11.26 | 3.4925 | 7.73E−06 |
| RBP1 | retinol binding protein 1_cellular | 11.22 | 3.4885 | 9.69E−27 |
| PPARGC1A | peroxisome proliferator-activated | 11.21 | 3.4872 | 5.02E−05 |
| | receptor gamma_coactivator 1 alpha | | | |
| TMEM200C | transmembrane protein 200C | 11.15 | 3.4784 | 1.09E−04 |
| PCDHA11 | protocadherin alpha 11 | 11.14 | 3.4777 | 8.36E−07 |
| PCDHA3 | protocadherin alpha 3 | 11.13 | 3.4768 | 2.60E−10 |
| LRFN5 | leucine rich repeat and fibronectin | 11.07 | 3.4686 | 5.35E−09 |
| | type III domain containing 5 | | | |
| SCGB3A2 | secretoglobin_family 3A_member | 10.82 | 3.4361 | 1.56E−04 |
| | 2 | | | |
| SCN2B | sodium channel_voltage gated_{—} | 10.81 | 3.4348 | 1.58E−04 |
| | type II beta subunit | | | |
| HMGA2 | high mobility group AT-hook 2 | 10.78 | 3.4309 | 4.79E−14 |
| TLL1 | tolloid-like 1 | 10.77 | 3.4296 | 3.71E−22 |
| PM20D2 | peptidase M20 domain containing 2 | 10.77 | 3.4292 | 2.19E−22 |
| PURG | purine-rich element binding protein | 10.72 | 3.4228 | 1.15E−06 |
| | G | | | |
| KLHL38 | kelch-like family member 38 | 10.68 | 3.4173 | 2.07E−06 |
| HIST1H2BH | histone cluster 1_H2bh | 10.68 | 3.4170 | 1.17E−12 |
| ITGB6 | integrin_beta 6 | 10.56 | 3.4000 | 9.77E−09 |
| AFF3 | AF4/FMR2 family_member 3 | 10.55 | 3.3986 | 3.23E−81 |
| ZBED2 | zinc finger_BED-type containing 2 | 10.49 | 3.3907 | 1.64E−06 |
| TRHDE | thyrotropin-releasing hormone | 10.40 | 3.3789 | 5.13E−05 |
| | degrading enzyme | | | |
| APBA2 | amyloid beta (A4) precursor protein- | 10.39 | 3.3775 | 2.69E−08 |
| | binding_family A_member 2 | | | |
| PCDHA4 | protocadherin alpha 4 | 10.36 | 3.3733 | 2.60E−08 |
| SMIM1 | small integral membrane protein 1 | 10.27 | 3.3608 | 6.15E−07 |
| | (Vel blood group) | | | |
| PIK3R3 | phosphoinositide-3-kinase_{—} | 10.19 | 3.3496 | 2.71E−34 |
| | regulatory subunit 3 (gamma) | | | |
| KALRN | kalirin_RhoGEF kinase | 10.03 | 3.3267 | 1.54E−34 |
| LOC728463 | NA | 10.01 | 3.3241 | 2.74E−04 |
| PTN | pleiotrophin | 9.96 | 3.3165 | 2.92E−06 |
| CLDN6 | claudin 6 | 9.95 | 3.3142 | 3.72E−07 |
| ASXL3 | additional sex combs like | 9.93 | 3.3111 | 1.05E−04 |
| | transcriptional regulator 3 | | | |
| KBTBD11 | kelch repeat and BTB (POZ) domain | 9.86 | 3.3023 | 2.00E−06 |
| | containing 11 | | | |
| GALNT14 | polypeptide N- | 9.86 | 3.3022 | 1.82E−09 |
| | acetylgalactosaminyltransferase 14 | | | |
| LOC440173 | uncharacterized LOC440173 | 9.86 | 3.3022 | 1.39E−04 |
| TLE4 | transducin-like enhancer of split 4 | 9.85 | 3.2996 | 2.87E−71 |
| NOX4 | NADPH oxidase 4 | 9.81 | 3.2948 | 1.77E−23 |
| EPHX4 | epoxide hydrolase 4 | 9.73 | 3.2823 | 1.50E−05 |
| DIO2 | deiodinase_iodothyronine_type_II | 9.68 | 3.2755 | 2.14E−05 |
| DNAJC6 | DnaJ (Hsp40) homolog_subfamily | 9.60 | 3.2634 | 7.55E−24 |
| | C_member 6 | | | |
| SLC16A12 | solute carrier family 16_member 12 | 9.60 | 3.2630 | 2.76E−06 |
| BCL11A | B-cell CLL/lymphoma 11A (zinc | 9.49 | 3.2467 | 4.04E−15 |
| | finger protein) | | | |
| ZNF608 | zinc finger protein 608 | 9.45 | 3.2402 | 2.33E−16 |
| PPAP2C | phosphatidic acid phosphatase type | 9.37 | 3.2285 | 1.22E−17 |
| | 2C | | | |
| IGSF3 | immunoglobulin superfamily_{—} | 9.29 | 3.2164 | 2.26E−38 |
| | member 3 | | | |
| COL18A1 | collagen_type XVIII_alpha 1 | 9.20 | 3.2021 | 2.63E−16 |
| ZNF732 | zinc finger protein 732 | 9.18 | 3.1988 | 3.05E−16 |
| NAALAD2 | N-acetylated alpha-linked acidic | 9.18 | 3.1979 | 4.47E−06 |
| | dipeptidase 2 | | | |
| EXOC3L2 | exocyst complex component 3-like 2 | 9.16 | 3.1959 | 8.87E−09 |
| JUP | junction plakoglobin | 9.14 | 3.1926 | 3.22E−24 |
| MSR1 | macrophage scavenger receptor 1 | 9.12 | 3.1888 | 4.36E−07 |
| TRIM58 | tripartite motif containing 58 | 9.03 | 3.1745 | 3.32E−25 |
| TMSB15A | thymosin beta 15a | 9.02 | 3.1728 | 2.04E−17 |
| MAPK15 | mitogen-activated protein kinase 15 | 9.00 | 3.1707 | 1.35E−05 |
| CELSR1 | cadherin_EGF LAG seven-pass G- | 9.00 | 3.1705 | 1.83E−15 |
| | type receptor 1 | | | |
| SEMA3D | sema domain_immunoglobulin | 8.96 | 3.1630 | 2.81E−06 |
| | domain (Ig)_short basic domain_{—} | | | |
| | secreted_(semaphorin) 3D | | | |
| SH3RF2 | SH3 domain containing ring finger 2 | 8.93 | 3.1586 | 5.06E−16 |
| MYPN | myopalladin | 8.81 | 3.1391 | 5.10E−11 |
| PKD1L1 | polycystic kidney disease 1 like 1 | 8.80 | 3.1377 | 1.21E−05 |
| PCDHA13 | protocadherin alpha 13 | 8.76 | 3.1317 | 4.55E−04 |
| PKNOX2 | PBX/knotted 1 homeobox 2 | 8.76 | 3.1317 | 7.68E−07 |
| ZIC5 | Zic family member 5 | 8.74 | 3.1277 | 3.70E−05 |
| LOC90246 | uncharacterized LOC90246 | 8.72 | 3.1251 | 4.62E−14 |
| SLC12A5 | solute carrier family 12 | 8.68 | 3.1175 | 1.98E−09 |
| | (potassium/chloride transporter)_{—} | | | |
| | member 5 | | | |
| PCDHB10 | protocadherin beta 10 | 8.67 | 3.1168 | 2.00E−16 |
| TMEM63C | transmembrane protein 63C | 8.65 | 3.1130 | 1.86E−09 |
| LYN | LYN proto-oncogene_Src family | 8.65 | 3.1127 | 9.22E−41 |
| | tyrosine kinase | | | |
| CHMP4C | charged multivesicular body protein | 8.61 | 3.1057 | 6.67E−06 |
| | 4C | | | |
| GPRIN2 | G protein regulated inducer of | 8.56 | 3.0977 | 1.13E−05 |
| | neurite outgrowth 2 | | | |
| TNS3 | tensin 3 | 8.56 | 3.0972 | 3.01E−30 |
| DOCK3 | dedicator of cytokinesis 3 | 8.55 | 3.0955 | 4.29E−21 |
| CPA4 | carboxypeptidase A4 | 8.54 | 3.0935 | 1.85E−05 |
| C1orf106 | chromosome 1 open reading frame | 8.53 | 3.0928 | 6.67E−10 |
| | 106 | | | |
| LOC339862 | uncharacterized LOC339862 | 8.51 | 3.0891 | 2.74E−04 |
| SLC6A6 | solute carrier family 6 | 8.47 | 3.0819 | 6.02E−44 |
| | (neurotransmitter transporter)_{—} | | | |
| | member 6 | | | |
| LPPR3 | lipid phosphate phosphatase-related | 8.43 | 3.0762 | 2.23E−10 |
| | protein type 3 | | | |
| BMF | Bc12 modifying factor | 8.43 | 3.0758 | 4.14E−79 |
| MDK | midkine (neurite growth-promoting | 8.43 | 3.0749 | 3.83E−52 |
| | factor 2) | | | |
| SBK1 | SH3 domain binding kinase 1 | 8.38 | 3.0668 | 8.81E−06 |
| ZNF676 | zinc finger protein 676 | 8.36 | 3.0643 | 2.95E−04 |
| SIM2 | single-minded family bHLH | 8.32 | 3.0570 | 6.97E−17 |
| | transcription factor 2 | | | |
| COL24A1 | collagen_type XXIV_alpha 1 | 8.31 | 3.0555 | 2.37E−06 |
| C14orf39 | chromosome 14 open reading frame | 8.29 | 3.0520 | 9.36E−04 |
| | 39 | | | |
| RTL1 | retrotransposon-like 1 | 8.29 | 3.0513 | 2.60E−06 |
| TUBB2B | tubulin_beta 2B class IIb | 8.29 | 3.0508 | 1.38E−04 |
| PDZD2 | PDZ domain containing 2 | 8.23 | 3.0409 | 1.60E−15 |
| SEMA6B | sema domain_transmembrane | 8.22 | 3.0388 | 2.53E−15 |
| | domain (TM)_and cytoplasmic | | | |
| | domain_(semaphorin) 6B | | | |
| KCTD8 | potassium channel tetramerization | 8.21 | 3.0380 | 8.62E−04 |
| | domain containing 8 | | | |
| FAM213A | family with sequence similarity | 8.19 | 3.0336 | 3.82E−06 |
| | 213_member A | | | |
| HRASLS | HRAS-like suppressor | 8.18 | 3.0326 | 2.51E−07 |
| TRIML2 | tripartite motif family-like 2 | 8.14 | 3.0253 | 8.36E−16 |
| CNIH2 | cornichon family AMPA receptor | 8.09 | 3.0166 | 1.42E−47 |
| | auxiliary protein 2 | | | |
| OCA2 | oculocutaneous albinism II | 8.01 | 3.0011 | 4.47E−04 |
| RNF165 | ring finger protein 165 | 8.01 | 3.0010 | 2.48E−04 |
| PTPRN2 | protein tyrosine phosphatase_{—} | 8.00 | 3.0000 | 3.98E−34 |
| | receptor type_N polypeptide 2 | | | |
| PIK3C2B | phosphatidylinositol-4-phosphate 3- | 7.99 | 2.9979 | 4.89E−53 |
| | kinase_catalytic subunit type 2 beta | | | |
| NFE2 | nuclear factor_erythroid 2 | 7.96 | 2.9927 | 1.85E−04 |
| PRND | prion protein 2 (dublet) | 7.95 | 2.9901 | 1.26E−03 |
| EGLN3 | egl-9 family hypoxia-inducible | 7.91 | 2.9828 | 7.72E−07 |
| | factor 3 | | | |
| SLC38A3 | solute carrier family 38_member 3 | 7.88 | 2.9781 | 6.75E−04 |
| IGF2BP3 | insulin-like growth factor 2 mRNA | 7.87 | 2.9762 | 5.96E−05 |
| | binding protein 3 | | | |
| RAB27B | RAB27B_member RAS oncogene | 7.84 | 2.9712 | 1.03E−11 |
| | family | | | |
| LINC00333 | long intergenic non-protein coding | 7.84 | 2.9702 | 4.05E−04 |
| | RNA 333 | | | |
| CYTL1 | cytokine-like 1 | 7.81 | 2.9650 | 3.54E−05 |
| FENDRR | FOXF1 adjacent non-coding | 7.78 | 2.9597 | 5.21E−04 |
| | developmental regulatory RNA | | | |
| WNK3 | WNK lysine deficient protein kinase | 7.76 | 2.9568 | 6.00E−09 |
| | 3 | | | |
| CDH10 | cadherin 10_type 2 (T2-cadherin) | 7.73 | 2.9498 | 2.09E−11 |
| GPRIN3 | GPRIN family member 3 | 7.71 | 2.9468 | 1.31E−03 |
| DOK2 | docking protein 2_56 kDa | 7.70 | 2.9440 | 2.85E−05 |
| TTYH2 | tweety family member 2 | 7.70 | 2.9440 | 1.49E−48 |
| SLC2A12 | solute carrier family 2 (facilitated | 7.66 | 2.9377 | 3.75E−16 |
| | glucose transporter)_member 12 | | | |
| DYSF | dysferlin | 7.65 | 2.9362 | 6.16E−12 |
| NRARP | NOTCH-regulated ankyrin repeat | 7.65 | 2.9355 | 6.67E−10 |
| | protein | | | |
| CELSR2 | cadherin_EGF LAG seven-pass G- | 7.65 | 2.9354 | 4.02E−13 |
| | type receptor 2 | | | |
| RAD21L1 | RAD21 cohesin complex component | 7.65 | 2.9350 | 4.40E−04 |
| | like 1 | | | |
| RAP1GAP2 | RAP1 GTPase activating protein 2 | 7.63 | 2.9309 | 1.35E−09 |
| OGDHL | oxoglutarate dehydrogenase-like | 7.56 | 2.9179 | 1.33E−16 |
| IGFBP7-AS1 | IGFBP7 antisense RNA 1 | 7.51 | 2.9092 | 7.05E−06 |
| PIANP | PILR alpha associated neural protein | 7.46 | 2.8994 | 2.59E−15 |
| TRABD2A | TraB domain containing 2A | 7.46 | 2.8991 | 7.41E−83 |
| FSIP2 | fibrous sheath interacting protein 2 | 7.46 | 2.8986 | 1.04E−03 |
| RASSF4 | Ras association (RalGDS/AF-6) | 7.42 | 2.8915 | 7.70E−31 |
| | domain family member 4 | | | |
| ABCA4 | ATP-binding cassette_sub-family A | 7.34 | 2.8764 | 8.46E−09 |
| | (ABC1)_member 4 | | | |
| PPP1R3A | protein phosphatase 1_regulatory | 7.33 | 2.8734 | 2.01E−03 |
| | subunit 3A | | | |
| ZBTB46 | zinc finger and BTB domain | 7.32 | 2.8724 | 1.25E−30 |
| | containing 46 | | | |
| CYP2S1 | cytochrome P450_family 2_{—} | 7.29 | 2.8668 | 2.98E−09 |
| | subfamily S_polypeptide 1 | | | |
| DIRC3 | disrupted in renal carcinoma 3 | 7.26 | 2.8600 | 9.57E−08 |
| COL9A3 | collagen_type IX_alpha 3 | 7.24 | 2.8559 | 4.41E−10 |
| MAMDC2 | MAM domain containing 2 | 7.20 | 2.8474 | 5.15E−18 |
| GIPC3 | GIPC PDZ domain containing | 7.20 | 2.8471 | 6.98E−09 |
| | family_member 3 | | | |
| DPYSL4 | dihydropyrimidinase-like 4 | 7.18 | 2.8445 | 3.72E−06 |
| DLX2 | distal-less homeobox 2 | 7.17 | 2.8429 | 1.60E−37 |
| TRIM67 | tripartite motif containing 67 | 7.16 | 2.8401 | 5.57E−07 |
| ADAMTS18 | ADAM metallopeptidase with | 7.13 | 2.8348 | 1.91E−03 |
| | thrombospondin type 1 motif_18 | | | |
| IGDCC4 | immunoglobulin superfamily_DCC | 7.12 | 2.8317 | 2.14E−18 |
| | subclass_member 4 | | | |
| EFNA2 | ephrin-A2 | 7.12 | 2.8313 | 1.23E−04 |
| CPVL | carboxypeptidase_vitellogenic-like | 7.11 | 2.8292 | 1.50E−08 |
| PCDHA8 | protocadherin alpha 8 | 7.09 | 2.8261 | 1.57E−03 |
| DBNDD1 | dysbindin (dystrobrevin binding | 7.09 | 2.8253 | 2.34E−11 |
| | protein 1) domain containing 1 | | | |
| DNER | delta/notch-like EGF repeat | 7.08 | 2.8239 | 7.46E−15 |
| | containing | | | |
| NPW | neuropeptide W | 7.07 | 2.8226 | 7.31E−25 |
| GNGT2 | guanine nucleotide binding protein | 7.03 | 2.8129 | 8.59E−07 |
| | (G protein)_gamma transducing | | | |
| | activity polypeptide 2 | | | |
| CDC42BPG | CDC42 binding protein kinase | 7.02 | 2.8124 | 4.40E−12 |
| | gamma (DMPK-like) | | | |
| FBN2 | fibrillin 2 | 7.01 | 2.8089 | 1.27E−29 |
| TPSG1 | tryptase gamma 1 | 6.97 | 2.8020 | 1.48E−03 |
| KCND1 | potassium channel_voltage gated | 6.96 | 2.7996 | 8.82E−34 |
| | Shal related subfamily D_member 1 | | | |
| KRT80 | keratin 80_type II | 6.95 | 2.7979 | 1.69E−16 |
| ST6GAL1 | ST6 beta-galactosamide alpha-2_6- | 6.90 | 2.7872 | 3.42E−59 |
| | sialyltranferase 1 | | | |
| EPPK1 | epiplakin 1 | 6.89 | 2.7849 | 2.02E−06 |
| HS6ST2 | heparan sulfate 6-O-sulfotransferase | 6.89 | 2.7836 | 2.41E−03 |
| | 2 | | | |
| OBSCN | obscurin_cytoskeletal calmodulin | 6.88 | 2.7826 | 2.91E−28 |
| | and titin-interacting RhoGEF | | | |
| CCDC68 | coiled-coil domain containing 68 | 6.88 | 2.7825 | 1.73E−22 |
| ZNF185 | zinc finger protein 185 (LIM | 6.87 | 2.7805 | 1.15E−04 |
| | domain) | | | |
| PCDHB9 | protocadherin beta 9 | 6.84 | 2.7748 | 1.21E−09 |
| SH3GL2 | SH3-domain GRB2-like 2 | 6.84 | 2.7736 | 3.07E−03 |
| LINC00707 | long intergenic non-protein coding | 6.81 | 2.7680 | 5.48E−04 |
| | RNA 707 | | | |
| GABRA5 | gamma-aminobutyric acid (GABA) | 6.78 | 2.7620 | 2.28E−24 |
| | A receptor_alpha 5 | | | |
| KRT8 | keratin 8_type II | 6.78 | 2.7605 | 1.07E−07 |
| RNF43 | ring finger protein 43 | 6.76 | 2.7576 | 3.24E−03 |
| SLC35F3 | solute carrier family 35_member F3 | 6.74 | 2.7536 | 7.42E−05 |
| SNCA | synuclein_alpha (non A4 | 6.68 | 2.7395 | 2.22E−03 |
| | component of amyloid precursor) | | | |
| CGN | cingulin | 6.65 | 2.7323 | 3.68E−05 |
| LOC100131289 | uncharacterized LOC100131289 | 6.62 | 2.7260 | 1.55E−03 |
| LOC100128885 | uncharacterized LOC100128885 | 6.60 | 2.7219 | 8.13E−11 |
| LOC653712 | intraflagellar transport 122 homolog | 6.59 | 2.7198 | 5.35E−18 |
| | (Chlamydomonas) pseudogene | | | |
| LLGL2 | lethal giant larvae homolog 2 | 6.58 | 2.7171 | 2.79E−10 |
| | (Drosophila) | | | |
| TRIM62 | tripartite motif containing 62 | 6.54 | 2.7097 | 7.99E−154 |
| AMZ1 | archaelysin family metallopeptidase | 6.54 | 2.7088 | 1.81E−70 |
| | 1 | | | |
| PDE3B | phosphodiesterase 3B_cGMP- | 6.54 | 2.7085 | 2.17E−05 |
| | inhibited | | | |
| IGDCC3 | immunoglobulin superfamily_DCC | 6.51 | 2.7021 | 1.17E−03 |
| | subclass_member 3 | | | |
| RAB38 | RAB38_member RAS oncogene | 6.48 | 2.6951 | 2.73E−05 |
| | family | | | |
| SFMBT2 | Scm-like with four mbt domains 2 | 6.47 | 2.6930 | 1.62E−13 |
| MEST | mesoderm specific transcript | 6.42 | 2.6817 | 3.56E−05 |
| MAP2K6 | mitogen-activated protein kinase | 6.31 | 2.6583 | 5.33E−06 |
| | kinase 6 | | | |
| TOX | thymocyte selection-associated high | 6.21 | 2.6352 | 1.98E−05 |
| | mobility group box | | | |
| GARNL3 | GTPase activating Rap/RanGAP | 6.21 | 2.6336 | 2.42E−05 |
| | domain-like 3 | | | |
| TRIM16L | tripartite motif containing 16-like | 6.20 | 2.6334 | 1.13E−18 |
| ABI3 | ABI family_member 3 | 6.20 | 2.6330 | 3.20E−33 |
| SHC4 | SHC (Src homology 2 domain | 6.20 | 2.6326 | 3.82E−11 |
| | containing) family_member 4 | | | |
| BFSP1 | beaded filament structural protein 1_{—} | 6.17 | 2.6255 | 3.20E−22 |
| | filensin | | | |
| FAXC | failed axon connections homolog | 6.17 | 2.6251 | 2.70E−16 |
| TBX1 | T-box 1 | 6.16 | 2.6234 | 1.74E−03 |
| PLS1 | plastin 1 | 6.15 | 2.6195 | 8.36E−16 |
| RGS9 | regulator of G-protein signaling 9 | 6.14 | 2.6177 | 9.91E−08 |
| NLRP3 | NLR family_pyrin domain | 6.13 | 2.6164 | 2.52E−04 |
| | containing 3 | | | |
| LOC101928775 | uncharacterized LOC101928775 | 6.13 | 2.6148 | 5.43E−03 |
| FAM84B | family with sequence similarity 84_{—} | 6.09 | 2.6074 | 4.97E−08 |
| | member B | | | |
| VSTM1 | V-set and transmembrane domain | 6.09 | 2.6073 | 5.51E−03 |
| | containing 1 | | | |
| RNF150 | ring finger protein 150 | 6.09 | 2.6064 | 1.69E−03 |
| KIF21B | kinesin family member 21B | 6.06 | 2.6002 | 2.72E−25 |
| ZNF702P | zinc finger protein 702_pseudogene | 6.05 | 2.5959 | 1.47E−10 |
| ITPRIPL1 | inositol 1_4_5-trisphosphate | 6.04 | 2.5955 | 1.98E−19 |
| | receptor interacting protein-like 1 | | | |
| ANKRD18B | ankyrin repeat domain 18B | 6.02 | 2.5907 | 1.70E−03 |
| SIX1 | SIX homeobox 1 | 6.02 | 2.5889 | 8.50E−09 |
| RUNX3 | runt-related transcription factor 3 | 6.00 | 2.5848 | 1.62E−12 |
| TNFRSF21 | tumor necrosis factor receptor | 5.98 | 2.5803 | 2.24E−24 |
| | superfamily_member 21 | | | |
| SUSD5 | sushi domain containing 5 | 5.98 | 2.5795 | 1.27E−03 |
| GRIP1 | glutamate receptor interacting | 5.96 | 2.5744 | 5.40E−05 |
| | protein 1 | | | |
| MEGF10 | multiple EGF-like-domains 10 | 5.94 | 2.5704 | 5.12E−03 |
| MGC2889 | uncharacterized protein MGC2889 | 5.94 | 2.5696 | 4.80E−03 |
| EDARADD | EDAR-associated death domain | 5.92 | 2.5663 | 2.25E−13 |
| FBXO16 | F-box protein 16 | 5.91 | 2.5642 | 6.29E−08 |
| VASH2 | vasohibin 2 | 5.90 | 2.5606 | 5.92E−08 |
| PCDHAC1 | protocadherin alpha subfamily C_1 | 5.88 | 2.5560 | 3.37E−03 |
| ADM5 | adrenomedullin 5 (putative) | 5.88 | 2.5552 | 4.05E−10 |
| FAM160A1 | family with sequence similarity | 5.86 | 2.5510 | 2.61E−03 |
| | 160 member A1 | | | |
| EFNB3 | ephrin-B3 | 5.86 | 2.5500 | 5.60E−13 |
| STK32B | serine/threonine kinase 32B | 5.85 | 2.5482 | 1.91E−83 |
| MYOZ1 | myozenin 1 | 5.82 | 2.5412 | 4.63E−04 |
| EGF | epidermal growth factor | 5.82 | 2.5398 | 8.06E−07 |
| FRRS1L | ferric-chelate reductase 1-like | 5.81 | 2.5387 | 5.23E−03 |
| CSRP2 | cysteine and glycine-rich protein 2 | 5.81 | 2.5386 | 1.37E−56 |
| FAM83F | family with sequence similarity 83_{—} | 5.78 | 2.5323 | 2.50E−03 |
| | member F | | | |
| LOC101929690 | NA | 5.78 | 2.5321 | 1.50E−03 |
| EPB41L4B | erythrocyte membrane protein band | 5.78 | 2.5303 | 1.25E−26 |
| | 4.1 like 4B | | | |
| APOE | apolipoprotein E | 5.76 | 2.5265 | 5.10E−11 |
| PCDHGC4 | protocadherin gamma subfamily C_{—} | 5.76 | 2.5249 | 8.40E−12 |
| | 4 | | | |
| GPR162 | G protein-coupled receptor 162 | 5.72 | 2.5166 | 1.36E−08 |
| SLC29A2 | solute carrier family 29 | 5.71 | 2.5131 | 7.20E−15 |
| | (equilibrative nucleoside | | | |
| | transporter)_member 2 | | | |
| GULP1 | GULP_engulfment adaptor PTB | 5.70 | 2.5107 | 9.81E−17 |
| | domain containing 1 | | | |
| AC093375.1 | NA | 5.69 | 2.5079 | 6.38E−03 |
| PIFO | primary cilia formation | 5.68 | 2.5048 | 3.68E−03 |
| GALNT3 | polypeptide N- | 5.67 | 2.5039 | 1.41E−05 |
| | acetylgalactosaminyltransferase 3 | | | |
| CBX2 | chromobox homolog 2 | 5.67 | 2.5031 | 4.47E−37 |
| PROC | protein C (inactivator of coagulation | 5.67 | 2.5029 | 8.18E−07 |
| | factors Va and VIIIa) | | | |
| CHD7 | chromodomain helicase DNA | 5.66 | 2.5004 | 1.63E−18 |
| | binding protein 7 | | | |
| VAC14-AS1 | VAC14 antisense RNA 1 | 5.66 | 2.5001 | 3.38E−05 |
| ISYNA1 | inositol-3-phosphate synthase 1 | 5.65 | 2.4986 | 1.68E−21 |
| FBXL16 | F-box and leucine-rich repeat | 5.64 | 2.4961 | 1.63E−07 |
| | protein 16 | | | |
| NKAIN4 | Na+/K+ transporting ATPase | 5.64 | 2.4951 | 3.47E−03 |
| | interacting 4 | | | |
| HID1 | HID1 domain containing | 5.63 | 2.4927 | 1.59E−04 |
| SYT12 | synaptotagmin XII | 5.62 | 2.4907 | 4.24E−03 |
| BEGAIN | brain-enriched guanylate kinase- | 5.61 | 2.4875 | 1.29E−07 |
| | associated | | | |
| OCIAD2 | OCIA domain containing 2 | 5.60 | 2.4850 | 9.91E−54 |
| FSD1 | fibronectin type III and SPRY | 5.59 | 2.4817 | 1.01E−24 |
| | domain containing 1 | | | |
| SCD5 | stearoyl-CoA desaturase 5 | 5.58 | 2.4813 | 1.61E−13 |
| PTCHD4 | patched domain containing 4 | 5.57 | 2.4767 | 6.76E−04 |
| OR2W3 | olfactory receptor_family 2_{—} | 5.55 | 2.4735 | 4.87E−07 |
| | subfamily W_member 3 | | | |
| PNMT | phenylethanolamine N- | 5.55 | 2.4733 | 3.44E−03 |
| | methyltransferase | | | |
| ZNF208 | zinc finger protein 208 | 5.51 | 2.4630 | 1.91E−04 |
| MYOZ3 | myozenin 3 | 5.50 | 2.4595 | 1.47E−20 |
| CPT1B | carnitine palmitoyltransferase 1B | 5.50 | 2.4587 | 5.67E−03 |
| | (muscle) | | | |
| KCNMA1 | potassium channel_calcium | 5.48 | 2.4541 | 1.81E−67 |
| | activated large conductance | | | |
| | subfamily M alpha_member 1 | | | |
| PALMD | palmdelphin | 5.47 | 2.4521 | 8.82E−05 |
| SYNGR1 | synaptogyrin 1 | 5.46 | 2.4485 | 1.69E−91 |
| DRP2 | dystrophin related protein 2 | 5.46 | 2.4482 | 5.80E−23 |
| CAPN14 | calpain 14 | 5.42 | 2.4384 | 3.28E−03 |
| SOX17 | SRY (sex determining region Y)- | 5.39 | 2.4314 | 8.94E−03 |
| | box 17 | | | |
| PTGES3L | prostaglandin E synthase 3 | 5.39 | 2.4308 | 2.45E−04 |
| | (cytosolic)-like | | | |
| KCTD4 | potassium channel tetramerization | 5.38 | 2.4287 | 2.05E−04 |
| | domain containing 4 | | | |
| PCDHA6 | protocadherin alpha 6 | 5.38 | 2.4282 | 3.90E−03 |
| LOC101927497 | uncharacterized LOC101927497 | 5.37 | 2.4259 | 7.44E−07 |
| TMEM184A | transmembrane protein 184A | 5.35 | 2.4201 | 5.40E−18 |
| DOCK4 | dedicator of cytokinesis 4 | 5.35 | 2.4184 | 6.49E−25 |
| THEMIS | thymocyte selection associated | 5.34 | 2.4177 | 1.06E−02 |
| HEY1 | hes-related family bHLH | 5.34 | 2.4169 | 1.31E−06 |
| | transcription factor with YRPW | | | |
| | motif 1 | | | |
| MKRN3 | makorin ring finger protein 3 | 5.34 | 2.4156 | 9.13E−13 |
| JAG2 | jagged 2 | 5.33 | 2.4144 | 2.59E−09 |
| LOC101927482 | uncharacterized LOC101927482 | 5.33 | 2.4137 | 2.19E−04 |
| RND2 | Rho family GTPase 2 | 5.32 | 2.4121 | 2.31E−15 |
| DSC2 | desmocollin 2 | 5.32 | 2.4107 | 1.56E−03 |
| CTXN1 | cortexin 1 | 5.31 | 2.4095 | 1.48E−11 |
| LOC100128076 | protein tyrosine phosphatase | 5.31 | 2.4080 | 2.30E−04 |
| | pseudogene | | | |
| KCNS1 | potassium voltage-gated channel_{—} | 5.30 | 2.4048 | 1.77E−19 |
| | modifier subfamily S_member 1 | | | |
| KCNMB4 | potassium channel subfamily M | 5.29 | 2.4042 | 4.48E−16 |
| | regulatory beta subunit 4 | | | |
| MCTP1 | multiple C2 domains_{—} | 5.28 | 2.4012 | 5.81E−06 |
| | transmembrane 1 | | | |
| SLC2A14 | solute carrier family 2 (facilitated | 5.26 | 2.3962 | 5.86E−04 |
| | glucose transporter)_member 14 | | | |
| MTL5 | metallothionein-like 5_testis- | 5.25 | 2.3921 | 1.23E−09 |
| | specific (tesmin) | | | |
| SLC16A4 | solute carrier family 16_member 4 | 5.24 | 2.3905 | 2.14E−63 |
| CARD10 | caspase recruitment domain family_{—} | 5.23 | 2.3856 | 3.39E−28 |
| | member 10 | | | |
| TMEM108 | transmembrane protein 108 | 5.21 | 2.3821 | 2.63E−05 |
| NETO2 | neuropilin (NRP) and tolloid (TLL)- | 5.19 | 2.3764 | 5.94E−37 |
| | like 2 | | | |
| CLDN16 | claudin 16 | 5.16 | 2.3679 | 1.34E−02 |
| SLC29A4 | solute carrier family 29 | 5.15 | 2.3656 | 1.71E−23 |
| | (equilibrative nucleoside | | | |
| | transporter)_member 4 | | | |
| ZBED9 | zinc finger_BED-type containing 9 | 5.15 | 2.3652 | 6.60E−10 |
| SLC22A31 | solute carrier family 22_member 31 | 5.15 | 2.3641 | 3.24E−03 |
| CCND2 | cyclin D2 | 5.13 | 2.3600 | 2.48E−26 |
| BEX1 | brain expressed_X-linked 1 | 5.13 | 2.3592 | 1.18E−02 |
| PPM1H | protein phosphatase_Mg2+/Mn2+ | 5.13 | 2.3592 | 5.26E−07 |
| | dependent_1H | | | |
| C7orf61 | chromosome 7 open reading frame | 5.13 | 2.3588 | 7.21E−06 |
| | 61 | | | |
| RGPD1 | RANBP2-like and GRIP domain | 5.13 | 2.3586 | 4.05E−03 |
| | containing 1 | | | |
| GPR143 | G protein-coupled receptor 143 | 5.13 | 2.3576 | 9.43E−03 |
| TNFRSF10C | tumor necrosis factor receptor | 5.10 | 2.3515 | 4.48E−07 |
| | superfamily_member 10c_decoy | | | |
| | without an intracellular domain | | | |
| MSI2 | musashi RNA-binding protein 2 | 5.10 | 2.3502 | 1.21E−79 |
| HIST1H3F | histone cluster 1_H3f | 5.09 | 2.3485 | 1.42E−02 |
| TRIM55 | tripartite motif containing 55 | 5.07 | 2.3425 | 9.83E−05 |
| LPAR3 | lysophosphatidic acid receptor 3 | 5.07 | 2.3411 | 5.33E−03 |
| LEPREL1 | prolyl 3-hydroxylase 2 | 5.03 | 2.3313 | 5.14E−05 |
| KCNN3 | potassium channel_calcium | 5.01 | 2.3251 | 7.84E−06 |
| | activated intermediate/small | | | |
| | conductance subfamily N alpha_{—} | | | |
| | member 3 | | | |

TABLE 8
Genes more highly expressed in AD-MSCs compared with HMCs

| | | | Log | |
|---|---|---|---|---|
| | | Fold | Fold | |
| Gene name | Description | Change | Change | p-Adj |
| TWIST2 | twist family bHLH transcription factor | −615.13 | −9.265 | 2.6E−153 |
| | 2 | | | |
| FGL2 | fibrinogen-like 2 | −521.90 | −9.028 | 1.5E−52 |
| PI16 | peptidase inhibitor 16 | −505.64 | −8.982 | 1.6E−78 |
| EMX2OS | EMX2 opposite strand/antisense RNA | −429.80 | −8.748 | 4.3E−56 |
| XIST | X inactive specific transcript (non- | −416.10 | −8.701 | 2.3E−269 |
| | protein coding) | | | |
| ISLR | immunoglobulin superfamily | −316.46 | −8.306 | 1.1E−26 |
| | containing leucine-rich repeat | | | |
| MEOX2 | mesenchyme homeobox 2 | −302.76 | −8.242 | 2.2E−45 |
| HAGLR | HOXD antisense growth-associated | −273.46 | −8.095 | 7.7E−39 |
| | long non-coding RNA | | | |
| FAM180A | family with sequence similarity 180_{—} | −260.12 | −8.023 | 3.2E−51 |
| | member A | | | |
| LINC00856 | long intergenic non-protein coding | −254.50 | −7.992 | 2.9E−36 |
| | RNA 856 | | | |
| EMX2 | empty spiracles homeobox 2 | −246.43 | −7.945 | 9.7E−54 |
| TNXB | tenascin XB | −240.63 | −7.911 | 7.3E−140 |
| HAS1 | hyaluronan synthase 1 | −233.59 | −7.868 | 4.5E−47 |
| HAS2 | hyaluronan synthase 2 | −209.41 | −7.710 | 8.3E−139 |
| TBX5-AS1 | TBX5 antisense RNA 1 | −202.78 | −7.664 | 1.8E−41 |
| BHMT2 | betaine--homocysteine S- | −195.84 | −7.614 | 8.1E−82 |
| | methyltransferase 2 | | | |
| HOXC5 | homeobox C5 | −185.04 | −7.532 | 1.1E−52 |
| COMP | cartilage oligomeric matrix protein | −182.83 | −7.514 | 3.3E−35 |
| DOK5 | docking protein 5 | −182.49 | −7.512 | 2.7E−154 |
| CSTA | cystatin A (stefin A) | −181.14 | −7.501 | 5.4E−32 |
| CCDC36 | coiled-coil domain containing 36 | −179.59 | −7.489 | 3.7E−42 |
| TPTEP1 | transmembrane phosphatase with | −175.65 | −7.457 | 1.4E−29 |
| | tensin homology pseudogene 1 | | | |
| XG | Xg blood group | −174.60 | −7.448 | 1.6E−37 |
| KRT14 | keratin 14_ type I | −170.06 | −7.410 | 9.0E−29 |
| NDNF | neuron-derived neurotrophic factor | −169.92 | −7.409 | 1.6E−46 |
| HTR2A | 5-hydroxytryptamine (serotonin) | −160.86 | −7.330 | 1.2E−34 |
| | receptor 2A_ G protein-coupled | | | |
| PSG5 | pregnancy specific beta-1-glycoprotein | −160.55 | −7.327 | 8.1E−76 |
| | 5 | | | |
| DCLK3 | doublecortin-like kinase 3 | −158.45 | −7.308 | 3.7E−29 |
| KCND2 | potassium channel_ voltage gated Shal | −148.30 | −7.212 | 4.1E−28 |
| | related subfamily D_ member 2 | | | |
| LINC01133 | long intergenic non-protein coding | −139.87 | −7.128 | 1.6E−31 |
| | RNA 1133 | | | |
| CNTN3 | contactin 3 (plasmacytoma associated) | −137.81 | −7.107 | 1.3E−66 |
| GPAT2 | glycerol-3-phosphate acyltransferase | −137.06 | −7.099 | 5.4E−37 |
| | 2_ mitochondrial | | | |
| HOXC6 | homeobox C6 | −136.95 | −7.098 | 0.0E+00 |
| KRBOX1 | KRAB box domain containing 1 | −136.24 | −7.090 | 9.5E−54 |
| ITGBL1 | integrin_ beta-like 1 (with EGF-like | −135.06 | −7.077 | 0.0E+00 |
| | repeat domains) | | | |
| PCDHGA12 | protocadherin gamma subfamily A_ 12 | −134.87 | −7.075 | 1.8E−210 |
| DMGDH | dimethylglycine dehydrogenase | −130.82 | −7.031 | 5.0E−36 |
| SGCG | sarcoglycan_ gamma (35 kDa | −130.78 | −7.031 | 1.1E−29 |
| | dystrophin-associated glycoprotein) | | | |
| HOXD3 | homeobox D3 | −130.60 | −7.029 | 3.0E−26 |
| HOXD8 | homeobox D8 | −127.97 | −7.000 | 8.4E−156 |
| EGFLAM | EGF-like_ fibronectin type III and | −127.49 | −6.994 | 2.4E−43 |
| | laminin G domains | | | |
| HOXD9 | homeobox D9 | −118.10 | −6.884 | 1.1E−41 |
| MASP1 | mannan-binding lectin serine peptidase | −115.18 | −6.848 | 3.1E−29 |
| | 1 (C4/C2 activating component of Ra- | | | |
| | reactive factor) | | | |
| OLFM1 | olfactomedin 1 | −113.80 | −6.830 | 2.5E−110 |
| ADRA2A | adrenoceptor alpha 2A | −109.79 | −6.779 | 2.0E−49 |
| HOXD4 | homeobox D4 | −109.17 | −6.770 | 1.2E−47 |
| ARHGAP20 | Rho GTPase activating protein 20 | −108.16 | −6.757 | 2.8E−48 |
| PRR15 | proline rich 15 | −107.72 | −6.751 | 2.2E−148 |
| PENK | proenkephalin | −103.97 | −6.700 | 1.4E−30 |
| MMP3 | matrix metallopeptidase 3 | −101.89 | −6.671 | 7.5E−36 |
| SFRP4 | secreted frizzled-related protein 4 | −100.96 | −6.658 | 1.1E−22 |
| SIM1 | single-minded family bHLH | −100.64 | −6.653 | 6.4E−36 |
| | transcription factor 1 | | | |
| TEKT4P2 | tektin 4 pseudogene 2 | −98.84 | −6.627 | 1.6E−37 |
| MYH2 | myosin_ heavy chain 2_ skeletal | −98.26 | −6.619 | 9.2E−25 |
| | muscle_ adult | | | |
| EN1 | engrailed homeobox 1 | −98.11 | −6.616 | 7.5E−95 |
| TBX5 | T-box 5 | −94.95 | −6.569 | 7.2E−31 |
| HOXC10 | homeobox C10 | −94.63 | −6.564 | 1.4E−43 |
| ABCC9 | ATP-binding cassette_ sub-family C | −89.87 | −6.490 | 7.9E−81 |
| | (CFTR/MRP)_ member 9 | | | |
| HOXC-AS2 | HOXC cluster antisense RNA 2 | −89.25 | −6.480 | 1.4E−29 |
| USP32P1 | ubiquitin specific peptidase 32 | −87.52 | −6.452 | 3.3E−25 |
| | pseudogene 1 | | | |
| FMOD | fibromodulin | −87.47 | −6.451 | 1.1E−75 |
| ABCA8 | ATP-binding cassette_ sub-family A | −87.45 | −6.450 | 3.1E−33 |
| | (ABC1)_ member 8 | | | |
| PDE1A | phosphodiesterase 1A_ calmodulin- | −86.65 | −6.437 | 3.6E−56 |
| | dependent | | | |
| COL15A1 | collagen_ type XV_ alpha 1 | −86.33 | −6.432 | 1.7E−142 |
| HOXC4 | homeobox C4 | −85.68 | −6.421 | 4.2E−84 |
| GSC | goosecoid homeobox | −85.63 | −6.420 | 2.9E−28 |
| IL13RA2 | interleukin 13 receptor_ alpha 2 | −84.87 | −6.407 | 1.1E−21 |
| LINC00968 | long intergenic non-protein coding | −83.09 | −6.377 | 4.8E−35 |
| | RNA 968 | | | |
| HOXD-AS2 | HOXD cluster antisense RNA 2 | −82.83 | −6.372 | 1.6E−67 |
| PAPPA2 | pappalysin 2 | −82.24 | −6.362 | 6.6E−35 |
| HOXC8 | homeobox C8 | −81.56 | −6.350 | 0.0E+00 |
| CCDC144B | coiled-coil domain containing 144B | −79.34 | −6.310 | 5.6E−35 |
| | (pseudogene) | | | |
| TMEM233 | transmembrane protein 233 | −74.43 | −6.218 | 2.6E−19 |
| HOXC9 | homeobox C9 | −74.28 | −6.215 | 2.5E−275 |
| FAM225B | family with sequence similarity 225_{—} | −74.15 | −6.212 | 1.9E−18 |
| | member B (non-protein coding) | | | |
| FGF7 | fibroblast growth factor 7 | −72.94 | −6.189 | 8.8E−55 |
| C2orf88 | chromosome 2 open reading frame 88 | −69.58 | −6.121 | 5.1E−41 |
| NFASC | neurofascin | −67.27 | −6.072 | 2.8E−158 |
| HSPB2 | heat shock 27 kDa protein 2 | −66.67 | −6.059 | 1.7E−95 |
| HOXA10-AS | HOXA10 antisense RNA | −64.54 | −6.012 | 5.4E−28 |
| HOXA7 | homeobox A7 | −63.72 | −5.994 | 1.8E−32 |
| USP32P2 | ubiquitin specific peptidase 32 | −63.62 | −5.991 | 3.0E−24 |
| | pseudogene 2 | | | |
| MCF2L | MCF.2 cell line derived transforming | −62.47 | −5.965 | 6.2E−44 |
| | sequence-like | | | |
| DCN | decorin | −60.95 | −5.929 | 9.3E−243 |
| PRSS12 | protease_ serine_ 12 (neurotrypsin_{—} | −59.56 | −5.896 | 6.0E−143 |
| | motopsin) | | | |
| LAMA2 | laminin_ alpha 2 | −59.38 | −5.892 | 2.7E−151 |
| RARRES2 | retinoic acid receptor responder | −59.19 | −5.887 | 8.3E−25 |
| | (tazarotene induced) 2 | | | |
| EYA2 | EYA transcriptional coactivator and | −58.85 | −5.879 | 4.3E−18 |
| | phosphatase 2 | | | |
| LINC01018 | long intergenic non-protein coding | −58.61 | −5.873 | 3.3E−16 |
| | RNA 1018 | | | |
| CLEC11A | C-type lectin domain family 11_{—} | −58.21 | −5.863 | 0.0E+00 |
| | member A | | | |
| CRLF1 | cytokine receptor-like factor 1 | −57.83 | −5.854 | 7.2E−39 |
| TRH | thyrotropin-releasing hormone | −57.47 | −5.845 | 6.7E−16 |
| LOC400043 | uncharacterized LOC400043 | −56.54 | −5.821 | 4.9E−49 |
| ASPN | asporin | −56.26 | −5.814 | 2.0E−26 |
| PRG4 | proteoglycan 4 | −56.25 | −5.814 | 3.7E−24 |
| LYNX1 | Ly6/neurotoxin 1 | −56.17 | −5.812 | 5.7E−40 |
| HOTAIRM1 | HOXA transcript antisense RNA_{—} | −55.20 | −5.787 | 1.6E−63 |
| | myeloid-specific 1 | | | |
| NUPR1 | nuclear protein_ transcriptional | −53.82 | −5.750 | 2.4E−182 |
| | regulator_ 1 | | | |
| CECR7 | cat eye syndrome chromosome region_{—} | −53.72 | −5.747 | 9.2E−17 |
| | candidate 7 (non-protein coding) | | | |
| GREM2 | gremlin 2_ DAN family BMP | −52.48 | −5.714 | 5.2E−78 |
| | antagonist | | | |
| ADAMTSL3 | ADAMTS-like 3 | −52.02 | −5.701 | 2.5E−16 |
| KCNE4 | potassium channel_ voltage gated | −51.90 | −5.698 | 2.1E−145 |
| | subfamily E regulatory beta subunit 4 | | | |
| PODN | podocan | −51.36 | −5.683 | 7.4E−182 |
| PRDM6 | PR domain containing 6 | −50.92 | −5.670 | 2.9E−21 |
| HOXA9 | homeobox A9 | −50.65 | −5.663 | 1.7E−69 |
| HSPB7 | heat shock 27 kDa protein family_{—} | −50.60 | −5.661 | 0.0E+00 |
| | member 7 (cardiovascular) | | | |
| MFAP5 | microfibrillar associated protein 5 | −47.76 | −5.578 | 2.6E−241 |
| WISP2 | WNT1 inducible signaling pathway | −46.57 | −5.541 | 3.2E−16 |
| | protein 2 | | | |
| PPAPDC3 | phosphatidic acid phosphatase type 2 | −46.47 | −5.538 | 9.0E−97 |
| | domain containing 3 | | | |
| KCNJ8 | potassium channel_ inwardly | −46.17 | −5.529 | 1.5E−148 |
| | rectifying subfamily J_ member 8 | | | |
| PRSS30P | protease_ serine_ 30_ pseudogene | −46.12 | −5.527 | 3.5E−14 |
| NINJ2 | ninjurin 2 | −45.86 | −5.519 | 2.8E−29 |
| TECTB | tectorin beta | −44.68 | −5.482 | 1.1E−13 |
| IRX5 | iroquois homeobox 5 | −44.28 | −5.468 | 8.4E−64 |
| CADPS | Ca++-dependent secretion activator | −44.19 | −5.466 | 2.2E−24 |
| LIMCH1 | LIM and calponin homology domains | −44.02 | −5.460 | 7.0E−23 |
| | 1 | | | |
| NR3C2 | nuclear receptor subfamily 3_ group | −44.00 | −5.459 | 3.2E−17 |
| | C_ member 2 | | | |
| CCDC89 | coiled-coil domain containing 89 | −43.76 | −5.452 | 7.3E−53 |
| DUXAP10 | double homeobox A pseudogene 10 | −43.60 | −5.446 | 1.3E−63 |
| S1PR1 | sphingosine-1-phosphate receptor 1 | −43.42 | −5.440 | 1.0E−30 |
| FNDC1 | fibronectin type III domain containing | −43.32 | −5.437 | 7.2E−18 |
| | 1 | | | |
| HOXA6 | homeobox A6 | −43.04 | −5.428 | 1.5E−16 |
| MIRLET7BHG | MIRLET7B host gene | −42.02 | −5.393 | 1.7E−61 |
| IRX3 | iroquois homeobox 3 | −41.92 | −5.390 | 2.6E−99 |
| WNT2 | wingless-type MMTV integration site | −41.90 | −5.389 | 4.3E−12 |
| | family member 2_{—} | | | |
| HAS2-AS1 | HAS2 antisense RNA 1 | −41.88 | −5.388 | 2.6E−25 |
| LOC643355 | uncharacterized LOC643355 | −41.85 | −5.387 | 1.2E−13 |
| SYBU | syntabulin (syntaxin-interacting) | −41.62 | −5.379 | 7.3E−101 |
| MB | myoglobin | −41.60 | −5.378 | 1.8E−13 |
| GYPE | glycophorin E (MNS blood group) | −41.46 | −5.374 | 3.2E−17 |
| CLEC2B | C-type lectin domain family 2 | −41.02 | −5.358 | 1.1E−17 |
| | member B | | | |
| HOXC-AS1 | HOXC cluster antisense RNA 1 | −40.92 | −5.355 | 4.6E−20 |
| MALL | mal_ T-cell differentiation protein-like | −40.81 | −5.351 | 6.2E−43 |
| HOXA11 | homeobox A11 | −40.54 | −5.341 | 3.4E−48 |
| RFX8 | RFX family member 8_ lacking RFX | −40.47 | −5.339 | 1.3E−55 |
| | DNA binding domain | | | |
| BMPER | BMP binding endothelial regulator | −39.88 | −5.318 | 1.2E−59 |
| KCTD12 | potassium channel tetramerization | −39.69 | −5.311 | 2.0E−40 |
| | domain containing 12 | | | |
| CH25H | cholesterol 25-hydroxylase | −39.23 | −5.294 | 5.3E−13 |
| ERG | v-ets avian erythroblastosis virus E26 | −38.73 | −5.275 | 3.9E−13 |
| | oncogene homolog | | | |
| CCL26 | chemokine (C-C motif) ligand 26 | −38.66 | −5.273 | 3.4E−27 |
| HOXA10 | homeobox A10 | −38.54 | −5.268 | 1.7E−144 |
| POMC | proopiomelanocortin | −38.34 | −5.261 | 9.8E−12 |
| LOC100996609 | NA | −38.33 | −5.260 | 1.6E−17 |
| TDRD9 | tudor domain containing 9 | −38.08 | −5.251 | 1.9E−13 |
| LOC100506834 | uncharacterized LOC100506834 | −37.86 | −5.243 | 2.5E−12 |
| HOXB7 | homeobox B7 | −37.72 | −5.237 | 5.8E−95 |
| KRT34 | keratin 34_ type I | −37.35 | −5.223 | 7.0E−63 |
| FRMPD1 | FERM and PDZ domain containing 1 | −37.23 | −5.218 | 1.9E−12 |
| BHMT | betaine--homocysteine S- | −37.16 | −5.216 | 3.8E−12 |
| | methyltransferase | | | |
| FAM198A | family with sequence similarity 198_{—} | −36.59 | −5.194 | 1.9E−12 |
| | member A | | | |
| PSTPIP1 | proline-serine-threonine phosphatase | −36.30 | −5.182 | 3.6E−19 |
| | interacting protein 1 | | | |
| HOXB-AS3 | HOXB cluster antisense RNA 3 | −35.85 | −5.164 | 7.0E−51 |
| TRABD2B | TraB domain containing 2B | −35.59 | −5.153 | 1.7E−13 |
| GALNT12 | polypeptide N- | −34.90 | −5.125 | 6.8E−63 |
| | acetylgalactosaminyltransferase 12 | | | |
| C8orf31 | chromosome 8 open reading frame 31 | −34.72 | −5.118 | 1.0E−23 |
| ZNF300P1 | zinc finger protein 300 pseudogene 1 | −34.71 | −5.117 | 2.1E−51 |
| | (functional) | | | |
| TNFRSF11B | tumor necrosis factor receptor | −34.56 | −5.111 | 2.2E−27 |
| | superfamily_ member 11b | | | |
| PLBD1 | phospholipase B domain containing 1 | −34.51 | −5.109 | 7.9E−33 |
| PPP1R14C | protein phosphatase 1_ regulatory | −34.18 | −5.095 | 1.5E−18 |
| | (inhibitor) subunit 14C | | | |
| MROH9 | maestro heat-like repeat family | −34.17 | −5.095 | 1.7E−11 |
| | member 9 | | | |
| HOXD1 | homeobox D1 | −34.15 | −5.094 | 7.3E−15 |
| HOXA4 | homeobox A4 | −33.55 | −5.068 | 2.5E−30 |
| LUM | lumican | −33.14 | −5.050 | 1.5E−72 |
| HOXB5 | homeobox B5 | −33.04 | −5.046 | 2.2E−39 |
| MR1 | major histocompatibility complex_{—} | −32.88 | −5.039 | 3.7E−63 |
| | class I-related | | | |
| TSKS | testis-specific serine kinase substrate | −32.74 | −5.033 | 6.0E−15 |
| SPATA22 | spermatogenesis associated 22 | −32.55 | −5.025 | 1.9E−11 |
| GIPC2 | GIPC PDZ domain containing family_{—} | −32.43 | −5.019 | 3.5E−34 |
| | member 2 | | | |
| FGF14 | fibroblast growth factor 14 | −31.99 | −5.000 | 6.2E−30 |
| HOXB6 | homeobox B6 | −31.84 | −4.993 | 1.9E−126 |
| HOXB4 | homeobox B4 | −31.80 | −4.991 | 3.2E−58 |
| BAIAP2L2 | BAI1-associated protein 2-like 2 | −31.62 | −4.983 | 5.1E−35 |
| HOXB3 | homeobox B3 | −31.37 | −4.971 | 2.0E−77 |
| TP53TG3D | TP53 target 3D | −31.36 | −4.971 | 6.5E−11 |
| HOXA3 | homeobox A3 | −31.31 | −4.969 | 2.0E−13 |
| POSTN | periostin_ osteoblast specific factor | −30.78 | −4.944 | 1.8E−38 |
| IRAK3 | interleukin-1 receptor-associated | −30.66 | −4.938 | 6.7E−68 |
| | kinase 3 | | | |
| TNFSF9 | tumor necrosis factor (ligand) | −30.57 | −4.934 | 3.9E−47 |
| | superfamily_ member 9 | | | |
| BEAN1 | brain expressed_ associated with | −30.15 | −4.914 | 2.3E−12 |
| | NEDD4_ 1 | | | |
| HOXC11 | homeobox C11 | −29.68 | −4.891 | 5.8E−12 |
| LRRK2 | leucine-rich repeat kinase 2 | −29.57 | −4.886 | 2.4E−26 |
| NRN1 | neuritin 1 | −29.56 | −4.886 | 2.1E−133 |
| LOC388780 | uncharacterized LOC388780 | −29.54 | −4.884 | 1.1E−10 |
| C3orf80 | chromosome 3 open reading frame 80 | −29.33 | −4.874 | 1.7E−18 |
| PINLYP | phospholipase A2 inhibitor and | −29.27 | −4.871 | 1.9E−27 |
| | LY6/PLAUR domain containing | | | |
| PLAC9 | placenta-specific 9 | −29.00 | −4.858 | 7.1E−184 |
| CHST8 | carbohydrate (N-acetylgalactosamine | −28.41 | −4.828 | 1.1E−11 |
| | 4-0) sulfotransferase 8 | | | |
| LOC100240735 | uncharacterized LOC100240735 | −28.35 | −4.825 | 1.5E−12 |
| TSHZ2 | teashirt zinc finger homeobox 2 | −28.01 | −4.808 | 6.3E−14 |
| PRR34 | proline rich 34 | −27.65 | −4.789 | 3.7E−10 |
| DNASE1L3 | deoxyribonuclease I-like 3 | −27.53 | −4.783 | 2.5E−09 |
| COL10A1 | collagen_ type X_ alpha 1 | −27.42 | −4.777 | 7.4E−12 |
| FPR1 | formyl peptide receptor 1 | −27.04 | −4.757 | 2.4E−12 |
| KCND3 | potassium channel_ voltage gated Shal | −26.98 | −4.754 | 7.2E−41 |
| | related subfamily D_ member 3 | | | |
| MRAP2 | melanocortin 2 receptor accessory | −26.90 | −4.750 | 2.8E−09 |
| | protein 2 | | | |
| MIR10B | microRNA 10b | −26.66 | −4.737 | 5.5E−10 |
| DLX3 | distal-less homeobox 3 | −26.66 | −4.737 | 2.4E−18 |
| PCSK9 | proprotein convertase subtilisin/kexin | −26.34 | −4.719 | 2.0E−20 |
| | type 9 | | | |
| ANGPTL1 | angiopoietin-like 1 | −26.14 | −4.708 | 6.3E−15 |
| CLIC3 | chloride intracellular channel 3 | −26.07 | −4.704 | 4.0E−17 |
| OSR2 | odd-skipped related transciption factor | −26.05 | −4.703 | 8.5E−19 |
| | 2 | | | |
| SORCS2 | sortilin-related VPS10 domain | −25.91 | −4.696 | 5.3E−28 |
| | containing receptor 2 | | | |
| HOXB2 | homeobox B2 | −25.67 | −4.682 | 1.6E−154 |
| LOC728613 | programmed cell death 6 pseudogene | −25.51 | −4.673 | 2.8E−41 |
| ADAMTS4 | ADAM metallopeptidase with | −25.45 | −4.670 | 1.8E−57 |
| | thrombospondin type 1 motif_ 4 | | | |
| NGFR | nerve growth factor receptor | −25.08 | −4.648 | 3.5E−13 |
| KCNK2 | potassium channel_ two pore domain | −24.80 | −4.632 | 8.3E−98 |
| | subfamily K_ member 2 | | | |
| GAS1 | growth arrest-specific 1 | −24.65 | −4.623 | 1.3E−61 |
| ABCA9 | ATP-binding cassette_ sub-family A | −24.63 | −4.622 | 6.9E−09 |
| | (ABC1)_ member 9 | | | |
| THRB | thyroid hormone receptor_ beta | −24.45 | −4.612 | 4.6E−19 |
| M1AP | meiosis 1 associated protein | −24.10 | −4.591 | 3.3E−14 |
| SLC7A8 | solute carrier family 7 (amino acid | −24.02 | −4.586 | 2.4E−12 |
| | transporter light chain_ L system)_{—} | | | |
| | member 8 | | | |
| ENPP2 | ectonucleotide | −23.98 | −4.584 | 2.7E−26 |
| | pyrophosphatase/phosphodiesterase 2 | | | |
| LOC102724224 | NA | −23.97 | −4.583 | 2.7E−28 |
| GABBR2 | gamma-aminobutyric acid (GABA) B | −23.97 | −4.583 | 3.3E−09 |
| | receptor_ 2 | | | |
| RASSF9 | Ras association (RalGDS/AF-6) | −23.96 | −4.583 | 4.4E−22 |
| | domain family (N-terminal) member 9 | | | |
| TRIM29 | tripartite motif containing 29 | −23.93 | −4.581 | 6.4E−09 |
| GGT8P | gamma-glutamyltransferase 8 | −23.83 | −4.574 | 5.7E−09 |
| | pseudogene | | | |
| FBLN5 | fibulin 5 | −23.70 | −4.567 | 0.0E+00 |
| HOXA5 | homeobox A5 | −23.63 | −4.563 | 1.1E−12 |
| EYA4 | EYA transcriptional coactivator and | −23.47 | −4.553 | 2.6E−11 |
| | phosphatase 4 | | | |
| GPC3 | glypican 3 | −23.38 | −4.547 | 1.5E−10 |
| HTR1F | 5-hydroxytryptamine (serotonin) | −23.32 | −4.543 | 1.6E−08 |
| | receptor 1F_ G protein-coupled | | | |
| LOC101928370 | uncharacterized LOC101928370 | −23.01 | −4.525 | 7.4E−10 |
| HOXA2 | homeobox A2 | −23.01 | −4.524 | 1.3E−09 |
| LOC102800310 | NA | −22.91 | −4.518 | 2.1E−08 |
| RHBDL2 | rhomboid_ veinlet-like 2 (Drosophila) | −22.89 | −4.517 | 7.4E−46 |
| ACTC1 | actin_ alpha_ cardiac muscle 1 | −22.82 | −4.512 | 1.5E−88 |
| ACOX2 | acyl-CoA oxidase 2_ branched chain | −22.68 | −4.503 | 3.9E−55 |
| RAET1E | retinoic acid early transcript 1E | −22.54 | −4.494 | 1.5E−13 |
| TNFAIP8L3 | tumor necrosis factor_ alpha-induced | −22.53 | −4.494 | 4.3E−87 |
| | protein 8-like 3 | | | |
| LRRC15 | leucine rich repeat containing 15 | −22.43 | −4.487 | 1.4E−10 |
| IL33 | interleukin 33 | −22.38 | −4.484 | 2.2E−12 |
| PTPN20B | protein tyrosine phosphatase_ non- | −22.28 | −4.477 | 1.3E−08 |
| | receptor type 20 | | | |
| RIPK3 | receptor-interacting serine-threonine | −22.28 | −4.477 | 5.6E−19 |
| | kinase 3 | | | |
| CHI3L1 | chitinase 3-like 1 (cartilage | −22.22 | −4.474 | 8.4E−13 |
| | glycoprotein-39) | | | |
| CNKSR2 | connector enhancer of kinase | −22.19 | −4.472 | 1.5E−19 |
| | suppressor of Ras 2 | | | |
| ZFYVE28 | zinc finger_ FYVE domain containing | −22.16 | −4.470 | 3.9E−42 |
| | 28 | | | |
| HMOX1 | heme oxygenase 1 | −22.07 | −4.464 | 3.1E−113 |
| FLG-AS1 | FLG antisense RNA 1 | −22.02 | −4.461 | 3.8E−08 |
| SGCD | sarcoglycan_ delta (35 kDa dystrophin- | −21.92 | −4.454 | 4.1E−20 |
| | associated glycoprotein) | | | |
| CD36 | CD36 molecule (thrombospondin | −21.67 | −4.437 | 4.8E−08 |
| | receptor) | | | |
| GPR133 | adhesion G protein-coupled receptor | −21.65 | −4.436 | 1.1E−59 |
| | D1 | | | |
| PTGIS | prostaglandin I2 (prostacyclin) | −21.63 | −4.435 | 9.9E−125 |
| | synthase | | | |
| PCDHGA4 | protocadherin gamma subfamily A_ 4 | −21.59 | −4.432 | 4.0E−22 |
| RAI2 | retinoic acid induced 2 | −21.54 | −4.429 | 5.9E−10 |
| LCN1 | lipocalin 1 | −21.52 | −4.428 | 3.8E−09 |
| ANKRD6 | ankyrin repeat domain 6 | −21.48 | −4.425 | 1.2E−26 |
| ADIRF | adipogenesis regulatory factor | −21.09 | −4.398 | 1.3E−21 |
| ISLR2 | immunoglobulin superfamily | −21.04 | −4.395 | 1.1E−26 |
| | containing leucine-rich repeat 2 | | | |
| FLG | filaggrin | −21.04 | −4.395 | 2.0E−08 |
| IBSP | integrin-binding sialoprotein | −20.92 | −4.387 | 1.0E−07 |
| ELN | elastin | −20.70 | −4.371 | 1.1E−56 |
| SALL4 | spalt-like transcription factor 4 | −20.68 | −4.370 | 1.2E−13 |
| TRPV3 | transient receptor potential cation | −20.62 | −4.366 | 3.7E−28 |
| | channel_ subfamily V_ member 3 | | | |
| PTGS1 | prostaglandin-endoperoxide synthase 1 | −20.61 | −4.365 | 0.0E+00 |
| | (prostaglandin G/H synthase and | | | |
| | cyclooxygenase) | | | |
| FGF18 | fibroblast growth factor 18 | −20.56 | −4.361 | 2.0E−17 |
| ZNF662 | zinc finger protein 662 | −20.47 | −4.356 | 3.3E−35 |
| KCNJ15 | potassium channel_ inwardly | −20.33 | −4.346 | 1.0E−35 |
| | rectifying subfamily J_ member 15 | | | |
| LINC01354 | long intergenic non-protein coding | −20.07 | −4.327 | 1.1E−09 |
| | RNA 1354 | | | |
| LGI2 | leucine-rich repeat LGI family_{—} | −20.02 | −4.323 | 5.3E−13 |
| | member 2 | | | |
| TIMP3 | TIMP metallopeptidase inhibitor 3 | −19.80 | −4.308 | 4.6E−92 |
| EDA | ectodysplasin A | −19.58 | −4.292 | 7.3E−24 |
| FAM225A | family with sequence similarity 225_{—} | −19.26 | −4.267 | 1.1E−11 |
| | member A (non-protein coding) | | | |
| ALS2CR11 | amyotrophic lateral sclerosis 2 | −19.16 | −4.260 | 8.9E−24 |
| | (juvenile) chromosome region_{—} | | | |
| | candidate 11 | | | |
| COX7A1 | cytochrome c oxidase subunit VIIa | −19.02 | −4.249 | 1.1E−46 |
| | polypeptide 1 (muscle) | | | |
| HCG4 | HLA complex group 4 (non-protein | −18.90 | −4.240 | 1.3E−07 |
| | coding) | | | |
| KLF14 | Kruppel-like factor 14 | −18.65 | −4.221 | 2.2E−07 |
| APOD | apolipoprotein D | −18.62 | −4.219 | 1.8E−07 |
| NOV | nephroblastoma overexpressed | −18.58 | −4.215 | 2.0E−49 |
| CLEC14A | C-type lectin domain family 14_{—} | −18.57 | −4.215 | 2.3E−07 |
| | member A | | | |
| CGREF1 | cell growth regulator with EF-hand | −18.43 | −4.204 | 5.5E−55 |
| | domain 1 | | | |
| NTF3 | neurotrophin 3 | −18.40 | −4.201 | 9.1E−29 |
| FOLR3 | folate receptor 3 (gamma) | −18.29 | −4.193 | 1.6E−09 |
| LOC100132077 | uncharacterized LOC100132077 | −18.27 | −4.192 | 1.8E−25 |
| WNT11 | wingless-type MMTV integration site | −18.08 | −4.177 | 6.9E−15 |
| | family_ member 11 | | | |
| CLIC6 | chloride intracellular channel 6 | −17.89 | −4.161 | 8.0E−17 |
| PRSS3 | protease_ serine_ 3 | −17.80 | −4.154 | 1.5E−09 |
| PSG2 | pregnancy specific beta-1-glycoprotein | −17.77 | −4.152 | 5.8E−07 |
| | 2 | | | |
| MFSD7 | major facilitator superfamily domain | −17.75 | −4.150 | 3.3E−51 |
| | containing 7 | | | |
| PIWIL4 | piwi-like RNA-mediated gene | −17.71 | −4.147 | 1.3E−23 |
| | silencing 4 | | | |
| MEGF6 | multiple EGF-like-domains 6 | −17.69 | −4.145 | 9.3E−50 |
| LINC01116 | long intergenic non-protein coding | −17.69 | −4.145 | 3.0E−41 |
| | RNA 1116 | | | |
| TLX2 | T-cell leukemia homeobox 2 | −17.25 | −4.108 | 7.0E−10 |
| GRID1 | glutamate receptor_ ionotropic_ delta 1 | −17.25 | −4.108 | 6.6E−07 |
| DLGAP1 | discs_ large (Drosophila) homolog- | −17.21 | −4.105 | 4.3E−07 |
| | associated protein 1 | | | |
| SPESP1 | sperm equatorial segment protein 1 | −17.05 | −4.092 | 1.2E−09 |
| NAALADL1 | N-acetylated alpha-linked acidic | −16.94 | −4.083 | 1.2E−101 |
| | dipeptidase-like 1 | | | |
| IL22RA1 | interleukin 22 receptor_ alpha 1 | −16.93 | −4.081 | 6.4E−07 |
| SNORD114-10 | small nucleolar RNA_ C/D box 114-10 | −16.91 | −4.080 | 5.8E−07 |
| PSG1 | pregnancy specific beta-1-glycoprotein | −16.89 | −4.078 | 7.5E−07 |
| | 1 | | | |
| LOC100130872 | uncharacterized LOC100130872 | −16.85 | −4.075 | 4.2E−26 |
| LPXN | leupaxin | −16.83 | −4.073 | 2.6E−133 |
| GSTM5 | glutathione S-transferase mu 5 | −16.82 | −4.072 | 1.5E−13 |
| NDUFA4L2 | NADH dehydrogenase (ubiquinone) 1 | −16.80 | −4.071 | 6.2E−10 |
| | alpha subcomplex_ 4-like 2 | | | |
| MYH13 | myosin_ heavy chain 13_ skeletal | −16.76 | −4.067 | 1.1E−06 |
| | muscle | | | |
| PCDHGA2 | protocadherin gamma subfamily A_ 2 | −16.75 | −4.066 | 2.3E−29 |
| HOXB-AS1 | HOXB cluster antisense RNA 1 | −16.48 | −4.043 | 3.4E−24 |
| ZFP92 | ZFP92 zinc finger protein | −16.47 | −4.042 | 1.1E−08 |
| GLYATL2 | glycine-N-acyltransferase-like 2 | −16.44 | −4.039 | 9.7E−10 |
| LIPC | lipase_ hepatic | −16.34 | −4.031 | 9.3E−07 |
| BMPR1B | bone morphogenetic protein receptor_{—} | −16.32 | −4.029 | 4.9E−22 |
| | type IB | | | |
| PTGES | prostaglandin E synthase | −16.31 | −4.028 | 1.5E−35 |
| S100P | S100 calcium binding protein P | −16.14 | −4.013 | 1.4E−06 |
| LINC00595 | long intergenic non-protein coding | −16.01 | −4.001 | 7.2E−08 |
| | RNA 595 | | | |
| SLC1A2 | solute carrier family 1 (glial high | −15.96 | −3.996 | 9.5E−08 |
| | affinity glutamate transporter)_{—} | | | |
| | member 2 | | | |
| AGMO | alkylglycerol monooxygenase | −15.91 | −3.992 | 1.7E−07 |
| BMP6 | bone morphogenetic protein 6 | −15.87 | −3.988 | 7.5E−15 |
| SLC1A1 | solute carrier family 1 | −15.85 | −3.987 | 8.9E−14 |
| | (neuronal/epithelial high affinity | | | |
| | glutamate transporter_ system Xag)_{—} | | | |
| | member 1 | | | |
| IGF1 | insulin-like growth factor 1 | −15.78 | −3.980 | 5.4E−11 |
| | (somatomedin C) | | | |
| IFNE | interferon_ epsilon | −15.73 | −3.976 | 1.1E−14 |
| SHCBP1L | SHC SH2-domain binding protein 1- | −15.70 | −3.972 | 1.2E−06 |
| | like | | | |
| OPCML | opioid binding protein/cell adhesion | −15.69 | −3.972 | 1.3E−13 |
| | molecule-like | | | |
| DKK1 | dickkopf WNT signaling pathway | −15.64 | −3.967 | 1.5E−120 |
| | inhibitor 1 | | | |
| ASTL | astacin-like metallo-endopeptidase | −15.62 | −3.965 | 1.6E−06 |
| | (M12 family) | | | |
| LDLRAD4 | low density lipoprotein receptor class | −15.61 | −3.964 | 1.1E−19 |
| | A domain containing 4 | | | |
| P2RY6 | pyrimidinergic receptor P2Y_ G- | −15.57 | −3.960 | 2.6E−11 |
| | protein coupled_ 6 | | | |
| FAM87B | family with sequence similarity 87_{—} | −15.49 | −3.953 | 1.2E−15 |
| | member B | | | |
| PLEKHH2 | pleckstrin homology domain | −15.47 | −3.952 | 2.2E−64 |
| | containing_ family H (with MyTH4 | | | |
| | domain) member 2 | | | |
| ALK | anaplastic lymphoma receptor tyrosine | −15.46 | −3.951 | 1.8E−06 |
| | kinase | | | |
| MKX | mohawk homeobox | −15.44 | −3.948 | 3.9E−07 |
| MT1A | metallothionein 1A | −15.39 | −3.944 | 3.1E−16 |
| SHANK1 | SH3 and multiple ankyrin repeat | −15.31 | −3.937 | 2.7E−18 |
| | domains 1 | | | |
| LOC150381 | NA | −15.30 | −3.936 | 1.4E−30 |
| ZNF503 | zinc finger protein 503 | −14.98 | −3.905 | 8.3E−59 |
| ZMYND12 | zinc finger_ MYND-type containing | −14.96 | −3.903 | 8.4E−10 |
| | 12 | | | |
| A4GALT | alpha 1_4-galactosyltransferase | −14.91 | −3.898 | 4.6E−49 |
| HOXA1 | homeobox A1 | −14.87 | −3.894 | 1.0E−21 |
| ADRA2C | adrenoceptor alpha 2C | −14.85 | −3.892 | 2.6E−15 |
| GALNT13 | polypeptide N- | −14.70 | −3.878 | 4.4E−06 |
| | acetylgalactosaminyltransferase 13 | | | |
| RASIP 1 | Ras interacting protein 1 | −14.68 | −3.875 | 2.8E−21 |
| CCDC85A | coiled-coil domain containing 85A | −14.61 | −3.869 | 2.0E−10 |
| PLCL1 | phospholipase C-like 1 | −14.56 | −3.864 | 8.5E−11 |
| KLF8 | Kruppel-like factor 8 | −14.54 | −3.862 | 1.3E−15 |
| FAM20A | family with sequence similarity 20_{—} | −14.53 | −3.861 | 1.4E−18 |
| | member A | | | |
| HOXA-AS3 | HOXA cluster antisense RNA 3 | −14.51 | −3.859 | 2.2E−10 |
| LMO3 | LIM domain only 3 (rhombotin-like 2) | −14.44 | −3.852 | 2.8E−07 |
| LOC100133669 | uncharacterized LOC100133669 | −14.39 | −3.847 | 7.4E−10 |
| SLC22A3 | solute carrier family 22 (organic cation | −14.37 | −3.845 | 4.1E−18 |
| | transporter)_ member 3 | | | |
| SSTR1 | somatostatin receptor 1 | −14.28 | −3.835 | 1.7E−08 |
| SBSN | suprabasin | −14.27 | −3.835 | 1.1E−43 |
| LY96 | lymphocyte antigen 96 | −14.24 | −3.832 | 1.9E−48 |
| FAM46C | family with sequence similarity 46_{—} | −14.18 | −3.826 | 1.1E−08 |
| | member C | | | |
| ATP8B4 | ATPase_ class I_ type 8B_ member 4 | −14.07 | −3.814 | 5.2E−06 |
| LINC00702 | long intergenic non-protein coding | −14.02 | −3.810 | 3.3E−16 |
| | RNA 702 | | | |
| ANPEP | alanyl (membrane) aminopeptidase | −14.00 | −3.807 | 1.8E−57 |
| MIR31HG | MIR31 host gene | −13.99 | −3.806 | 1.7E−100 |
| ESPNL | espin-like | −13.85 | −3.791 | 1.8E−09 |
| FLJ12825 | uncharacterized LOC440101 | −13.84 | −3.791 | 3.6E−11 |
| KLF4 | Kruppel-like factor 4 (gut) | −13.73 | −3.779 | 1.2E−61 |
| KCNK15 | potassium channel_ two pore domain | −13.67 | −3.773 | 3.9E−08 |
| | subfamily K_ member 15 | | | |
| IL1RN | interleukin 1 receptor antagonist | −13.65 | −3.771 | 2.0E−07 |
| CACNB4 | calcium channel_ voltage-dependent_{—} | −13.65 | −3.771 | 8.5E−07 |
| | beta 4 subunit | | | |
| PPAP2B | phosphatidic acid phosphatase type 2B | −13.65 | −3.770 | 1.3E−64 |
| NEFM | neurofilament_ medium polypeptide | −13.53 | −3.758 | 1.3E−06 |
| KLF17 | Kruppel-like factor 17 | −13.51 | −3.756 | 1.1E−07 |
| CNGA3 | cyclic nucleotide gated channel alpha 3 | −13.50 | −3.755 | 9.6E−10 |
| ROS1 | ROS proto-oncogene 1 _ receptor | −13.44 | −3.749 | 4.4E−09 |
| | tyrosine kinase | | | |
| PTX3 | pentraxin 3_ long | −13.36 | −3.740 | 4.2E−21 |
| BRINP1 | bone morphogenetic protein/retinoic | −13.33 | −3.736 | 1.0E−05 |
| | acid inducible neural-specific 1 | | | |
| RGL3 | ral guanine nucleotide dissociation | −13.23 | −3.726 | 8.6E−06 |
| | stimulator-like 3 | | | |
| DEPTOR | DEP domain containing MTOR- | −13.21 | −3.723 | 1.2E−48 |
| | interacting protein | | | |
| ADH1C | alcohol dehydrogenase 1C (class I)_{—} | −13.16 | −3.718 | 1.1E−05 |
| | gamma polypeptide | | | |
| ADAMTS2 | ADAM metallopeptidase with | −13.14 | −3.716 | 3.0E−156 |
| | thrombospondin type 1 motif_ 2 | | | |
| CASP10 | caspase 10_ apoptosis-related cysteine | −13.13 | −3.715 | 2.2E−31 |
| | peptidase | | | |
| LINC00398 | long intergenic non-protein coding | −13.13 | −3.714 | 9.1E−06 |
| | RNA 398 | | | |
| TFPI2 | tissue factor pathway inhibitor 2 | −13.09 | −3.710 | 7.5E−08 |
| PLXDC2 | plexin domain containing 2 | −13.07 | −3.708 | 1.7E−08 |
| SYT7 | synaptotagmin VII | −12.97 | −3.697 | 1.5E−22 |
| GPC6 | glypican 6 | −12.96 | −3.696 | 4.6E−51 |
| GGT5 | gamma-glutamyltransferase 5 | −12.94 | −3.694 | 9.6E−10 |
| INMT | indolethylamine N-methyltransferase | −12.89 | −3.688 | 6.5E−06 |
| PTGDS | prostaglandin D2 synthase 21 kDa | −12.86 | −3.685 | 3.5E−09 |
| | (brain) | | | |
| CHRD | chordin | −12.79 | −3.677 | 1.0E−38 |
| PLA2G5 | phospholipase A2_ group V | −12.73 | −3.670 | 7.8E−08 |
| PTGER3 | prostaglandin E receptor 3 (subtype | −12.66 | −3.662 | 3.5E−15 |
| | EP3) | | | |
| RGS22 | regulator of G-protein signaling 22 | −12.64 | −3.660 | 1.4E−05 |
| CARD6 | caspase recruitment domain family_{—} | −12.59 | −3.654 | 5.0E−75 |
| | member 6 | | | |
| ANKRD30B | ankyrin repeat domain 30B | −12.58 | −3.653 | 4.9E−07 |
| NPY4R | neuropeptide Y receptor Y4 | −12.46 | −3.639 | 9.9E−07 |
| P2RY2 | purinergic receptor P2Y_ G-protein | −12.42 | −3.635 | 1.2E−06 |
| | coupled_ 2 | | | |
| HRCT1 | histidine rich carboxyl terminus 1 | −12.41 | −3.634 | 1.9E−08 |
| CCDC144A | coiled-coil domain containing 144A | −12.37 | −3.629 | 1.8E−07 |
| MEIS1 | Meis homeobox 1 | −12.33 | −3.624 | 5.6E−85 |
| DLEU7 | deleted in lymphocytic leukemia_ 7 | −12.30 | −3.620 | 8.7E−07 |
| ZNF385D | zinc finger protein 385D | −12.27 | −3.617 | 3.7E−16 |
| HOXB8 | homeobox B8 | −12.26 | −3.616 | 9.9E−27 |
| PCDHGA9 | protocadherin gamma subfamily A_ 9 | −12.25 | −3.614 | 3.0E−23 |
| DHRS3 | dehydrogenase/reductase (SDR family) | −12.17 | −3.605 | 2.7E−43 |
| | member 3 | | | |
| C4BPB | complement component 4 binding | −12.16 | −3.604 | 3.4E−05 |
| | protein_ beta | | | |
| ANKRD2 | ankyrin repeat domain 2 (stretch | −12.15 | −3.603 | 2.0E−19 |
| | responsive muscle) | | | |
| PHYHIP | phytanoyl-CoA 2-hydroxylase | −12.15 | −3.603 | 3.2E−10 |
| | interacting protein | | | |
| PPP2R2C | protein phosphatase 2_ regulatory | −12.11 | −3.598 | 5.3E−07 |
| | subunit B_ gamma | | | |
| AKR1C2 | aldo-keto reductase family 1_ member | −12.09 | −3.596 | 3.6E−174 |
| | C2 | | | |
| THNSL2 | threonine synthase-like 2 (S. | −12.08 | −3.594 | 5.4E−27 |
| | cerevisiae) | | | |
| PID1 | phosphotyrosine interaction domain | −12.07 | −3.593 | 1.8E−117 |
| | containing 1 | | | |
| PSORS1C1 | psoriasis susceptibility 1 candidate 1 | −12.03 | −3.588 | 9.8E−07 |
| CPXM2 | carboxypeptidase X (M14 family)_{—} | −11.97 | −3.581 | 6.1E−11 |
| | member 2 | | | |
| TNFAIP6 | tumor necrosis factor_ alpha-induced | −11.96 | −3.580 | 4.6E−09 |
| | protein 6 | | | |
| DMRT2 | doublesex and mab-3 related | −11.93 | −3.577 | 1.1E−08 |
| | transcription factor 2 | | | |
| PCDHGB3 | protocadherin gamma subfamily B_ 3 | −11.87 | −3.569 | 7.2E−18 |
| TMTC2 | transmembrane and tetratricopeptide | −11.84 | −3.566 | 2.1E−61 |
| | repeat containing 2 | | | |
| C2orf81 | chromosome 2 open reading frame 81 | −11.84 | −3.565 | 7.5E−65 |
| KANK4 | KN motif and ankyrin repeat domains | −11.81 | −3.562 | 2.8E−05 |
| | 4 | | | |
| SEL1L2 | sel-1 suppressor of lin-12-like 2 (C. | −11.80 | −3.561 | 3.2E−05 |
| | elegans) | | | |
| HOXC13 | homeobox C13 | −11.80 | −3.561 | 4.5E−05 |
| NR4A2 | nuclear receptor subfamily 4_ group | −11.74 | −3.554 | 1.2E−18 |
| | A_ member 2 | | | |
| FLRT2 | fibronectin leucine rich transmembrane | −11.74 | −3.553 | 3.3E−14 |
| | protein 2 | | | |
| SCRG1 | stimulator of chondrogenesis 1 | −11.71 | −3.550 | 1.5E−41 |
| LTBP2 | latent transforming growth factor beta | −11.70 | −3.549 | 9.9E−194 |
| | binding protein 2 | | | |
| SPON1 | spondin 1_ extracellular matrix protein | −11.65 | −3.543 | 1.8E−84 |
| SYNDIG1 | synapse differentiation inducing 1 | −11.63 | −3.540 | 2.2E−09 |
| MMRN2 | multimerin 2 | −11.57 | −3.532 | 6.7E−17 |
| EDNRB | endothelin receptor type B | −11.55 | −3.530 | 4.5E−05 |
| GRIA3 | glutamate receptor_ ionotropic_{—} | −11.54 | −3.528 | 2.1E−38 |
| | AMPA 3 | | | |
| SOD3 | superoxide dismutase 3_ extracellular | −11.53 | −3.527 | 1.7E−09 |
| SAMD3 | sterile alpha motif domain containing 3 | −11.37 | −3.507 | 9.7E−08 |
| SUSD3 | sushi domain containing 3 | −11.32 | −3.500 | 3.7E−30 |
| PCOLCE2 | procollagen C-endopeptidase enhancer | −11.28 | −3.496 | 7.1E−65 |
| | 2 | | | |
| C1QL3 | complement component 1_ q | −11.23 | −3.489 | 6.5E−11 |
| | subcomponent-like 3 | | | |
| SUSD2 | sushi domain containing 2 | −11.21 | −3.487 | 1.5E−06 |
| C1S | complement component 1_ s | −11.20 | −3.485 | 5.1E−125 |
| | subcomponent | | | |
| PRELP | proline/arginine-rich end leucine-rich | −11.17 | −3.481 | 8.7E−25 |
| | repeat protein | | | |
| CDA | cytidine deaminase | −11.15 | −3.479 | 2.5E−53 |
| PTPRD | protein tyrosine phosphatase_ receptor | −11.09 | −3.471 | 5.5E−07 |
| | type_ D | | | |
| ZDHHC15 | zinc finger_ DHHC-type containing 15 | −10.98 | −3.456 | 6.4E−06 |
| APOA1 | apolipoprotein A-I | −10.96 | −3.454 | 1.2E−08 |
| FHAD1 | forkhead-associated (FHA) | −10.96 | −3.454 | 2.0E−08 |
| | phosphopeptide binding domain 1 | | | |
| HIST1H1E | histone cluster 1_ H1e | −10.93 | −3.450 | 5.6E−05 |
| LOC100507642 | uncharacterized LOC100507642 | −10.92 | −3.449 | 3.1E−13 |
| CFD | complement factor D (adipsin) | −10.91 | −3.448 | 2.5E−23 |
| LOC100507540 | NA | −10.91 | −3.447 | 4.6E−20 |
| RTN1 | reticulon 1 | −10.90 | −3.447 | 4.4E−07 |
| ADH1B | alcohol dehydrogenase 1B (class I)_{—} | −10.85 | −3.440 | 7.8E−05 |
| | beta polypeptide | | | |
| CCL28 | chemokine (C-C motif) ligand 28 | −10.83 | −3.437 | 1.6E−05 |
| HOTAIR | HOX transcript antisense RNA | −10.79 | −3.432 | 3.2E−06 |
| LOC100505718 | NA | −10.76 | −3.427 | 1.3E−08 |
| RNF212 | ring finger protein 212 | −10.63 | −3.411 | 2.9E−16 |
| FIBCD1 | fibrinogen C domain containing 1 | −10.61 | −3.407 | 1.3E−28 |
| EFCAB1 | EF-hand calcium binding domain 1 | −10.60 | −3.406 | 2.2E−08 |
| LOC101059948 | uncharacterized LOC101059948 | −10.56 | −3.400 | 9.5E−06 |
| PCDH18 | protocadherin 18 | −10.53 | −3.397 | 1.0E−39 |
| CPNE8 | copine VIII | −10.51 | −3.394 | 1.5E−80 |
| TIMP1 | TIMP metallopeptidase inhibitor 1 | −10.49 | −3.390 | 0.0E+00 |
| TINAGL 1 | tubulointerstitial nephritis antigen-like | −10.39 | −3.377 | 7.6E−06 |
| | 1 | | | |
| C10orf11 | chromosome 10 open reading frame 11 | −10.27 | −3.360 | 2.1E−06 |
| PCDHGB5 | protocadherin gamma subfamily B_ 5 | −10.25 | −3.358 | 3.2E−102 |
| P2RX1 | purinergic receptor P2X_ ligand gated | −10.24 | −3.356 | 2.2E−05 |
| | ion channel_ 1 | | | |
| RPLP0P2 | ribosomal protein_ large_ P0 | −10.22 | −3.353 | 2.0E−07 |
| | pseudogene 2 | | | |
| HOXA11-AS | HOXA11 antisense RNA | −10.22 | −3.353 | 1.2E−11 |
| COL21A1 | collagen_ type XXI_ alpha 1 | −10.22 | −3.353 | 5.8E−05 |
| ESM1 | endothelial cell-specific molecule 1 | −10.20 | −3.351 | 1.5E−06 |
| FAM106A | family with sequence similarity 106_{—} | −10.19 | −3.350 | 1.1E−04 |
| | member A | | | |
| GHDC | GH3 domain containing | −10.17 | −3.347 | 6.2E−96 |
| LOC654342 | lymphocyte-specific protein 1 | −10.15 | −3.344 | 6.6E−147 |
| | pseudogene | | | |
| GAS7 | growth arrest-specific 7 | −10.07 | −3.332 | 2.5E−36 |
| FAM124A | family with sequence similarity 124A | −10.06 | −3.331 | 1.3E−05 |
| ITGB2-AS1 | ITGB2 antisense RNA 1 | −10.06 | −3.330 | 1.1E−06 |
| ZNF280A | zinc finger protein 280A | −10.04 | −3.328 | 1.4E−04 |
| MEDAG | mesenteric estrogen-dependent | −10.04 | −3.327 | 5.5E−17 |
| | adipogenesis | | | |
| DNAH2 | dynein_ axonemal_ heavy chain 2 | −9.99 | −3.320 | 4.8E−05 |
| WNT4 | wingless-type MMTV integration site | −9.96 | −3.317 | 4.4E−05 |
| | family_ member 4 | | | |
| COL12A1 | collagen_ type XII_ alpha 1 | −9.89 | −3.306 | 8.1E−35 |
| DMKN | dermokine | −9.87 | −3.303 | 6.9E−14 |
| SLC1A7 | solute carrier family 1 (glutamate | −9.83 | −3.297 | 3.5E−06 |
| | transporter)_ member 7 | | | |
| COL8A2 | collagen_ type VIII_ alpha 2 | −9.80 | −3.292 | 6.4E−09 |
| MYOM3 | myomesin 3 | −9.77 | −3.288 | 9.5E−28 |
| EPDR1 | ependymin related 1 | −9.76 | −3.287 | 2.2E−30 |
| TMEM155 | transmembrane protein 155 | −9.71 | −3.279 | 1.1E−08 |
| PODNL1 | podocan-like 1 | −9.71 | −3.279 | 5.8E−47 |
| PITX1 | paired-like homeodomain 1 | −9.68 | −3.275 | 2.3E−25 |
| IL20RA | interleukin 20 receptor_ alpha | −9.68 | −3.274 | 5.1E−05 |
| GPR4 | G protein-coupled receptor 4 | −9.67 | −3.274 | 5.5E−05 |
| GPX3 | glutathione peroxidase 3 | −9.67 | −3.273 | 5.3E−09 |
| C5orf27 | long intergenic non-protein coding | −9.67 | −3.273 | 2.3E−05 |
| | RNA 1554 | | | |
| CYP1B1 | cytochrome P450_ family 1_{—} | −9.64 | −3.269 | 3.2E−14 |
| | subfamily B_ polypeptide 1 | | | |
| TEK | TEK tyrosine kinase_ endothelial | −9.63 | −3.267 | 1.4E−61 |
| KRT13 | keratin 13_ type I | −9.60 | −3.263 | 5.5E−06 |
| NEFL | neurofilament_ light polypeptide | −9.58 | −3.260 | 4.5E−07 |
| BDKRB1 | bradykinin receptor B1 | −9.55 | −3.256 | 7.0E−39 |
| LINC01140 | long intergenic non-protein coding | −9.53 | −3.253 | 4.2E−17 |
| | RNA 1140 | | | |
| SEMA7A | semaphorin 7A_ GPI membrane | −9.53 | −3.252 | 2.6E−85 |
| | anchor (John Milton Hagen blood | | | |
| | group) | | | |
| PCDHGA7 | protocadherin gamma subfamily A_ 7 | −9.47 | −3.244 | 3.5E−20 |
| ZNF503-AS2 | ZNF503 antisense RNA 2 | −9.44 | −3.239 | 5.5E−25 |
| MMP12 | matrix metallopeptidase 12 | −9.43 | −3.238 | 2.5E−05 |
| ANKRD37 | ankyrin repeat domain 37 | −9.43 | −3.238 | 3.0E−38 |
| KRT81 | keratin 81_ type II | −9.40 | −3.233 | 4.2E−26 |
| AADAC | arylacetamide deacetylase | −9.40 | −3.232 | 6.1E−05 |
| PARP15 | poly (ADP-ribose) polymerase family_{—} | −9.33 | −3.223 | 9.8E−08 |
| | member 15 | | | |
| FAM90A1 | family with sequence similarity 90_{—} | −9.33 | −3.222 | 2.4E−04 |
| | member A1 | | | |
| OXCT2 | 3-oxoacid CoA transferase 2 | −9.33 | −3.222 | 4.6E−06 |
| SLC22A15 | solute carrier family 22_ member 15 | −9.29 | −3.215 | 1.2E−47 |
| SAA1 | serum amyloid A1 | −9.26 | −3.212 | 2.6E−04 |
| ANKRD65 | ankyrin repeat domain 65 | −9.25 | −3.210 | 6.6E−07 |
| UBE2QL1 | ubiquitin-conjugating enzyme E2Q | −9.24 | −3.208 | 5.9E−05 |
| | family-like 1 | | | |
| LHB | luteinizing hormone beta polypeptide | −9.22 | −3.205 | 5.5E−06 |
| SLC9A9 | solute carrier family 9_ subfamily A | −9.19 | −3.200 | 3.1E−26 |
| | (NHE9_ cation proton antiporter 9)_{—} | | | |
| | member 9 | | | |
| PRDM8 | PR domain containing 8 | −9.18 | −3.198 | 5.7E−119 |
| MAGOH2 | mago homolog 2_ pseudogene | −9.17 | −3.197 | 3.2E−04 |
| ICAM2 | intercellular adhesion molecule 2 | −9.16 | −3.195 | 1.2E−07 |
| NECAB2 | N-terminal EF-hand calcium binding | −9.13 | −3.191 | 1.1E−06 |
| | protein 2 | | | |
| MDGA1 | MAM domain containing | −9.13 | −3.191 | 9.3E−99 |
| | glycosylphosphatidylinositol anchor 1 | | | |
| BCL6B | B-cell CLL/lymphoma 6_ member B | −9.09 | −3.185 | 1.6E−05 |
| HSD11B1 | hydroxysteroid (11-beta) | −9.09 | −3.184 | 1.2E−05 |
| | dehydrogenase 1 | | | |
| DIRAS3 | DIRAS family_ GTP-binding RAS- | −9.05 | −3.177 | 7.7E−06 |
| | like 3 | | | |
| MOB3B | MOB kinase activator 3B | −9.03 | −3.175 | 6.7E−19 |
| ITM2A | integral membrane protein 2A | −9.02 | −3.173 | 3.8E−04 |
| CRYAB | crystallin_ alpha B | −9.01 | −3.171 | 1.1E−11 |
| HLA-F-AS1 | HLA-F antisense RNA 1 | −9.00 | −3.170 | 2.2E−17 |
| LINC00578 | long intergenic non-protein coding | −8.99 | −3.168 | 3.2E−04 |
| | RNA 578 | | | |
| DUSP2 | dual specificity phosphatase 2 | −8.95 | −3.163 | 6.4E−84 |
| FAM228A | family with sequence similarity 228_{—} | −8.94 | −3.161 | 1.2E−04 |
| | member A | | | |
| PLSCR4 | phospholipid scramblase 4 | −8.94 | −3.160 | 1.0E−65 |
| CD97 | adhesion G protein-coupled receptor | −8.87 | −3.148 | 3.3E−70 |
| | E5 | | | |
| KCNE1 | potassium channel_ voltage gated | −8.84 | −3.144 | 1.2E−07 |
| | subfamily E regulatory beta subunit 1 | | | |
| PCSK1 | proprotein convertase subtilisin/kexin | −8.79 | −3.135 | 3.2E−06 |
| | type 1 | | | |
| ZNF558 | zinc finger protein 558 | −8.78 | −3.134 | 1.3E−52 |
| CXCL6 | chemokine (C-X-C motif) ligand 6 | −8.77 | −3.132 | 3.5E−05 |
| KCNS3 | potassium voltage-gated channel_{—} | −8.73 | −3.125 | 7.4E−13 |
| | modifier subfamily S_ member 3 | | | |
| CD14 | CD14 molecule | −8.67 | −3.116 | 4.4E−07 |
| FLJ38576 | uncharacterized LOC651430 | −8.66 | −3.114 | 6.1E−05 |
| VTN | vitronectin | −8.65 | −3.113 | 4.3E−04 |
| EBF2 | early B-cell factor 2 | −8.64 | −3.111 | 7.4E−20 |
| MIR503 | microRNA 503 | −8.61 | −3.107 | 1.4E−11 |
| CHRDL2 | chordin-like 2 | −8.57 | −3.099 | 5.2E−04 |
| ACADL | acyl-CoA dehydrogenase_ long chain | −8.56 | −3.098 | 5.3E−04 |
| HCRTR1 | hypocretin (orexin) receptor 1 | −8.54 | −3.095 | 3.8E−05 |
| KCNC4-AS1 | KCNC4 antisense RNA 1 (head to | −8.51 | −3.088 | 7.1E−05 |
| | head) | | | |
| PVRL4 | poliovirus receptor-related 4 | −8.49 | −3.085 | 1.0E−07 |
| FRY | furry homolog (Drosophila) | −8.47 | −3.082 | 7.6E−11 |
| ITIH5 | inter-alpha-trypsin inhibitor heavy | −8.45 | −3.080 | 6.6E−04 |
| | chain family_ member 5 | | | |
| GSTO2 | glutathione S-transferase omega 2 | −8.42 | −3.075 | 3.8E−22 |
| LOC101927524 | NA | −8.42 | −3.074 | 6.3E−04 |
| PODXL | podocalyxin-like | −8.37 | −3.065 | 2.3E−09 |
| STXBP5L | syntaxin binding protein 5-like | −8.36 | −3.063 | 1.1E−04 |
| NR4A1 | nuclear receptor subfamily 4_ group | −8.36 | −3.063 | 2.6E−72 |
| | A_ member 1 | | | |
| CD55 | CD55 molecule_ decay accelerating | −8.32 | −3.057 | 2.0E−67 |
| | factor for complement (Cromer blood | | | |
| | group) | | | |
| FMO3 | flavin containing monooxygenase 3 | −8.28 | −3.049 | 2.1E−04 |
| ZG16B | zymogen granule protein 16B | −8.26 | −3.047 | 5.1E−05 |
| CHN2 | chimerin 2 | −8.24 | −3.043 | 7.0E−12 |
| FPR2 | formyl peptide receptor 2 | −8.18 | −3.033 | 2.0E−04 |
| COL5A3 | collagen_ type V_ alpha 3 | −8.18 | −3.032 | 3.6E−13 |
| TNFRSF14 | tumor necrosis factor receptor | −8.18 | −3.031 | 5.1E−10 |
| | superfamily_ member 14 | | | |
| PAQR9 | progestin and adipoQ receptor family | −8.17 | −3.031 | 2.5E−05 |
| | member IX | | | |
| LOC101927229 | uncharacterized LOC101927229 | −8.17 | −3.030 | 5.0E−04 |
| MME | membrane metallo-endopeptidase | −8.12 | −3.022 | 1.3E−12 |
| FZD1 | frizzled class receptor 1 | −8.11 | −3.020 | 6.2E−29 |
| COL6A6 | collagen_ type VI_ alpha 6 | −8.11 | −3.020 | 2.1E−04 |
| PRG2 | proteoglycan 2_ bone marrow (natural | −8.11 | −3.020 | 5.6E−04 |
| | killer cell activator_ eosinophil granule | | | |
| | major basic protein) | | | |
| PLD5 | phospholipase D family_ member 5 | −8.09 | −3.016 | 9.1E−04 |
| CCDC64B | coiled-coil domain containing 64B | −8.06 | −3.011 | 7.9E−04 |
| MIR503HG | MIR503 host gene | −8.05 | −3.010 | 9.4E−18 |
| SULF1 | sulfatase 1 | −8.05 | −3.009 | 1.7E−31 |
| SDHAP3 | succinate dehydrogenase complex_{—} | −8.05 | −3.009 | 9.2E−13 |
| | subunit A_ flavoprotein pseudogene 3 | | | |
| DACT1 | dishevelled-binding antagonist of beta- | −8.05 | −3.009 | 3.6E−50 |
| | catenin 1 | | | |
| C3 | complement component 3 | −8.02 | −3.004 | 3.9E−15 |
| ABI3BP | ABI family_ member 3 (NESH) | −8.00 | −3.001 | 4.3E−87 |
| | binding protein | | | |
| ANKH | ANKH inorganic pyrophosphate | −7.97 | −2.994 | 6.5E−31 |
| | transport regulator | | | |
| RADIL | Ras association and DIL domains | −7.96 | −2.992 | 1.5E−25 |
| ZNF454 | zinc finger protein 454 | −7.93 | −2.987 | 2.7E−06 |
| KRTAP1-5 | keratin associated protein 1-5 | −7.93 | −2.986 | 4.6E−05 |
| SUPT20HL1 | suppressor of Ty 20 homolog (S. | −7.92 | −2.986 | 4.5E−04 |
| | cerevisiae)-like 1 | | | |
| DPT | dermatopontin | −7.88 | −2.979 | 1.1E−03 |
| CHST15 | carbohydrate (N-acetylgalactosamine | −7.88 | −2.978 | 1.1E−22 |
| | 4-sulfate 6-O) sulfotransferase 15 | | | |
| OLFML1 | olfactomedin-like 1 | −7.86 | −2.974 | 5.2E−07 |
| MT1M | metallothionein 1M | −7.85 | −2.973 | 1.3E−28 |
| AKR1C1 | aldo-keto reductase family 1_ member | −7.84 | −2.971 | 2.0E−88 |
| | C1 | | | |
| TLE2 | transducin-like enhancer of split 2 | −7.84 | −2.970 | 5.6E−41 |
| PIGZ | phosphatidylinositol glycan anchor | −7.83 | −2.969 | 5.9E−32 |
| | biosynthesis_ class Z | | | |
| KRT16 | keratin 16_ type I | −7.82 | −2.967 | 2.2E−09 |
| CAPN3 | calpain 3 | −7.78 | −2.960 | 3.0E−18 |
| LOC100506385 | NA | −7.78 | −2.960 | 4.5E−14 |
| TBX18 | T-box 18 | −7.74 | −2.952 | 3.1E−119 |
| SOCS2-AS1 | SOCS2 antisense RNA 1 | −7.73 | −2.950 | 7.4E−13 |
| DLX4 | distal-less homeobox 4 | −7.71 | −2.947 | 1.1E−03 |
| PF4V1 | platelet factor 4 variant 1 | −7.71 | −2.947 | 1.1E−03 |
| LOC729041 | NA | −7.70 | −2.944 | 4.7E−04 |
| XYLT1 | xylosyltransferase I | −7.69 | −2.944 | 7.1E−65 |
| C1R | complement component 1_ r | −7.69 | −2.942 | 2.3E−23 |
| | subcomponent | | | |
| XAF1 | XIAP associated factor 1 | −7.68 | −2.940 | 1.7E−30 |
| RBPMS2 | RNA binding protein with multiple | −7.67 | −2.939 | 2.0E−22 |
| | splicing 2 | | | |
| SLC22A23 | solute carrier family 22_ member 23 | −7.67 | −2.938 | 1.0E−29 |
| RAB3IL1 | RAB3A interacting protein (rabin3)- | −7.66 | −2.937 | 4.6E−55 |
| | like 1 | | | |
| MPV17L | MPV17 mitochondrial membrane | −7.64 | −2.933 | 7.0E−07 |
| | protein-like | | | |
| CSF3 | colony stimulating factor 3 | −7.64 | −2.933 | 1.2E−03 |
| | (granulocyte) | | | |
| TRPM2 | transient receptor potential cation | −7.62 | −2.931 | 3.4E−05 |
| | channel_ subfamily M_ member 2 | | | |
| KRT33B | keratin 33B_ type I | −7.61 | −2.929 | 1.3E−14 |
| EID3 | EP300 interacting inhibitor of | −7.60 | −2.927 | 2.7E−30 |
| | differentiation 3 | | | |
| CES1 | carboxylesterase 1 | −7.59 | −2.925 | 9.9E−04 |
| ACSL5 | acyl-CoA synthetase long-chain family | −7.59 | −2.924 | 3.7E−14 |
| | member 5 | | | |
| CTSK | cathepsin K | −7.58 | −2.922 | 1.6E−18 |
| LINC00654 | long intergenic non-protein coding | −7.54 | −2.914 | 2.0E−26 |
| | RNA 654 | | | |
| F8 | coagulation factor VIII_ procoagulant | −7.53 | −2.913 | 2.6E−23 |
| | component | | | |
| MAGEB17 | melanoma antigen family B17 | −7.52 | −2.911 | 1.5E−03 |
| SLIT3 | slit guidance ligand 3 | −7.52 | −2.911 | 4.7E−230 |
| ZXDA | zinc finger_ X-linked duplicated A | −7.52 | −2.911 | 1.3E−44 |
| HAR1A | highly accelerated region 1A (non- | −7.49 | −2.905 | 2.9E−04 |
| | protein coding) | | | |
| IFI27 | interferon_ alpha-inducible protein 27 | −7.47 | −2.902 | 4.2E−34 |
| SPOCK1 | sparc/osteonectin_ cwcv and kazal-like | −7.47 | −2.901 | 3.8E−50 |
| | domains proteoglycan (testican) 1 | | | |
| FLJ43879 | FLJ43879 protein | −7.46 | −2.900 | 1.4E−03 |
| GPR150 | G protein-coupled receptor 150 | −7.46 | −2.898 | 1.1E−13 |
| DDO | D-aspartate oxidase | −7.45 | −2.898 | 1.6E−03 |
| JOSD2 | Josephin domain containing 2 | −7.44 | −2.895 | 1.4E−100 |
| ANKRD35 | ankyrin repeat domain 35 | −7.43 | −2.893 | 2.1E−41 |
| LINC00482 | long intergenic non-protein coding | −7.41 | −2.889 | 1.5E−03 |
| | RNA 482 | | | |
| TDRD1 | tudor domain containing 1 | −7.38 | −2.883 | 6.7E−04 |
| VCAN | versican | −7.37 | −2.882 | 3.8E−166 |
| OGN | osteoglycin | −7.37 | −2.882 | 8.3E−04 |
| S100A4 | S100 calcium binding protein A4 | −7.35 | −2.878 | 5.2E−09 |
| SHANK2 | SH3 and multiple ankyrin repeat | −7.35 | −2.877 | 1.8E−35 |
| | domains 2 | | | |
| POU5F1 | POU class 5 homeobox 1 | −7.35 | −2.877 | 3.4E−13 |
| CALB2 | calbindin 2 | −7.34 | −2.876 | 3.3E−04 |
| ECM2 | extracellular matrix protein 2_ female | −7.34 | −2.875 | 3.9E−08 |
| | organ and adipocyte specific | | | |
| WNT9A | wingless-type MMTV integration site | −7.32 | −2.872 | 9.3E−05 |
| | family_ member 9A | | | |
| LCE2A | late cornified envelope 2A | −7.32 | −2.872 | 1.8E−03 |
| IGFBP3 | insulin-like growth factor binding | −7.32 | −2.871 | 8.2E−48 |
| | protein 3 | | | |
| ANK2 | ankyrin 2_ neuronal | −7.28 | −2.864 | 9.4E−61 |
| ELOVL3 | ELOVL fatty acid elongase 3 | −7.28 | −2.863 | 5.0E−07 |
| MAB21L1 | mab-21-like 1 (C. elegans) | −7.27 | −2.863 | 9.4E−28 |
| ADCY4 | adenylate cyclase 4 | −7.27 | −2.862 | 2.0E−07 |
| RORA | RAR-related orphan receptor A | −7.27 | −2.861 | 1.2E−17 |
| MFAP4 | microfibrillar-associated protein 4 | −7.25 | −2.857 | 1.3E−27 |
| ZDHHC23 | zinc finger_ DHHC-type containing 23 | −7.22 | −2.852 | 9.9E−25 |
| SLC2A9 | solute carrier family 2 (facilitated | −7.20 | −2.848 | 5.5E−11 |
| | glucose transporter)_ member 9 | | | |
| SLC14A1 | solute carrier family 14 (urea | −7.19 | −2.847 | 1.8E−03 |
| | transporter)_ member 1 (Kidd blood | | | |
| | group) | | | |
| LRRC6 | leucine rich repeat containing 6 | −7.17 | −2.842 | 1.2E−08 |
| C15orf59 | chromosome 15 open reading frame 59 | −7.16 | −2.839 | 2.0E−08 |
| PRRX2 | paired related homeobox 2 | −7.15 | −2.838 | 6.5E−295 |
| C11orf91 | chromosome 11 open reading frame 91 | −7.15 | −2.838 | 2.6E−16 |
| LRRN4CL | LRRN4 C-terminal like | −7.12 | −2.832 | 7.3E−09 |
| FLRT1 | fibronectin leucine rich transmembrane | −7.12 | −2.832 | 4.3E−13 |
| | protein 1 | | | |
| PSG3 | pregnancy specific beta-1-glycoprotein | −7.11 | −2.830 | 1.9E−03 |
| | 3 | | | |
| CR1L | complement component (3b/4b) | −7.11 | −2.829 | 8.4E−04 |
| | receptor 1-like | | | |
| ABCA6 | ATP-binding cassette_ sub-family A | −7.06 | −2.820 | 1.5E−04 |
| | (ABC1)_ member 6 | | | |
| ADRA2B | adrenoceptor alpha 2B | −7.06 | −2.819 | 2.0E−03 |
| TPTE2P6 | transmembrane phosphoinositide 3- | −7.05 | −2.817 | 2.1E−03 |
| | phosphatase and tensin homolog 2 | | | |
| | pseudogene 6 | | | |
| MYH15 | myosin_ heavy chain 15 | −7.04 | −2.815 | 9.5E−13 |
| ZFP3 | ZFP3 zinc finger protein | −7.03 | −2.813 | 2.4E−44 |
| THEM6 | thioesterase superfamily member 6 | −7.02 | −2.811 | 1.5E−13 |
| MOXD1 | monooxygenase_ DBH-like 1 | −7.01 | −2.809 | 5.1E−14 |
| FBLN7 | fibulin 7 | −7.00 | −2.808 | 9.6E−18 |
| LOC728819 | NA | −7.00 | −2.808 | 8.2E−05 |
| EPHA1-AS1 | EPHA1 antisense RNA 1 | −6.99 | −2.806 | 2.2E−03 |
| PRL | prolactin | −6.99 | −2.805 | 2.3E−03 |
| PSG4 | pregnancy specific beta-1-glycoprotein | −6.96 | −2.799 | 1.3E−03 |
| | 4 | | | |
| LOC646762 | uncharacterized LOC646762 | −6.96 | −2.798 | 4.4E−40 |
| MIR497HG | mir-497-195 cluster host gene | −6.95 | −2.796 | 9.7E−10 |
| PTER | phosphotriesterase related | −6.94 | −2.795 | 3.8E−37 |
| ADAM12 | ADAM metallopeptidase domain 12 | −6.94 | −2.794 | 2.2E−231 |
| ADH6 | alcohol dehydrogenase 6 (class V) | −6.93 | −2.794 | 1.3E−04 |
| FAM66D | family with sequence similarity 66_{—} | −6.91 | −2.790 | 1.3E−07 |
| | member D | | | |
| GUCY1A2 | guanylate cyclase 1_ soluble_ alpha 2 | −6.90 | −2.787 | 8.6E−04 |
| MAPK13 | mitogen-activated protein kinase 13 | −6.87 | −2.781 | 3.9E−55 |
| PLCL2 | phospholipase C-like 2 | −6.87 | −2.780 | 1.7E−41 |
| POU3F2 | POU class 3 homeobox 2 | −6.86 | −2.778 | 9.8E−04 |
| DDX43 | DEAD (Asp-Glu-Ala-Asp) box | −6.84 | −2.774 | 2.6E−05 |
| | polypeptide 43 | | | |
| HIST2H2BA | histone cluster 2_ H2ba (pseudogene) | −6.84 | −2.774 | 1.8E−12 |
| IVL | involucrin | −6.83 | −2.772 | 2.7E−03 |
| DOK7 | docking protein 7 | −6.82 | −2.770 | 1.7E−04 |
| MUC13 | mucin 13_ cell surface associated | −6.81 | −2.767 | 2.8E−03 |
| FAM198B | family with sequence similarity 198_{—} | −6.79 | −2.763 | 2.1E−31 |
| | member B | | | |
| TRAPPC3L | trafficking protein particle complex 3- | −6.78 | −2.761 | 2.7E−03 |
| | like | | | |
| PIWIL2 | piwi-like RNA-mediated gene | −6.77 | −2.760 | 1.3E−03 |
| | silencing 2 | | | |
| RNF112 | ring finger protein 112 | −6.76 | −2.757 | 5.0E−11 |
| LINC01060 | long intergenic non-protein coding | −6.75 | −2.756 | 2.7E−12 |
| | RNA 1060 | | | |
| PCDHGB8P | protocadherin gamma subfamily B_ 8 | −6.75 | −2.754 | 5.8E−04 |
| | pseudogene | | | |
| SOST | sclerostin | −6.74 | −2.753 | 1.8E−03 |
| FAM167B | family with sequence similarity 167_{—} | −6.72 | −2.749 | 2.2E−17 |
| | member B | | | |
| IL21R | interleukin 21 receptor | −6.72 | −2.748 | 4.0E−64 |
| DDIT4L | DNA-damage-inducible transcript 4- | −6.71 | −2.746 | 2.3E−05 |
| | like | | | |
| C19orf81 | chromosome 19 open reading frame 81 | −6.71 | −2.745 | 2.8E−03 |
| AGTR1 | angiotensin II receptor_ type 1 | −6.70 | −2.745 | 1.8E−09 |
| SCUBE3 | signal peptide_ CUB domain_ EGF- | −6.69 | −2.742 | 9.5E−81 |
| | like 3 | | | |
| PDE2A | phosphodiesterase 2A_ cGMP- | −6.69 | −2.742 | 3.8E−04 |
| | stimulated | | | |
| MMP8 | matrix metallopeptidase 8 | −6.67 | −2.739 | 5.0E−04 |
| SHOX2 | short stature homeobox 2 | −6.66 | −2.737 | 6.0E−41 |
| DPY19L2P3 | DPY19L2 pseudogene 3 | −6.64 | −2.731 | 3.0E−03 |
| NPAS1 | neuronal PAS domain protein 1 | −6.63 | −2.730 | 4.2E−17 |
| FAM87A | family with sequence similarity 87_{—} | −6.63 | −2.729 | 3.3E−03 |
| | member A | | | |
| CEMIP | cell migration inducing protein_{—} | −6.61 | −2.725 | 3.4E−07 |
| | hyaluronan binding | | | |
| C1QTNF3 | C1q and tumor necrosis factor related | −6.58 | −2.718 | 1.0E−05 |
| | protein 3 | | | |
| ADAMTSL1 | ADAMTS-like 1 | −6.58 | −2.718 | 5.1E−33 |
| TSPEAR-AS1 | TSPEAR antisense RNA 1 | −6.55 | −2.713 | 1.1E−03 |
| ASCL2 | achaete-scute family bHLH | −6.52 | −2.705 | 5.9E−04 |
| | transcription factor 2 | | | |
| MYH3 | myosin_ heavy chain 3_ skeletal | −6.51 | −2.703 | 3.2E−13 |
| | muscle_ embryonic | | | |
| RPSAP52 | ribosomal protein SA pseudogene 52 | −6.50 | −2.701 | 4.2E−06 |
| KCNJ2-AS1 | KCNJ2 antisense RNA 1 (head to | −6.49 | −2.697 | 3.6E−03 |
| | head) | | | |
| LINC00961 | long intergenic non-protein coding | −6.47 | −2.695 | 2.7E−08 |
| | RNA 961 | | | |
| LINC01123 | long intergenic non-protein coding | −6.45 | −2.689 | 1.1E−06 |
| | RNA 1123 | | | |
| TBX15 | T-box 15 | −6.44 | −2.688 | 4.9E−09 |
| MCOLN3 | mucolipin 3 | −6.43 | −2.686 | 3.8E−03 |
| ROR2 | receptor tyrosine kinase-like orphan | −6.43 | −2.684 | 2.4E−04 |
| | receptor 2 | | | |
| DPP4 | dipeptidyl-peptidase 4 | −6.40 | −2.678 | 2.3E−07 |
| GPC4 | glypican 4 | −6.39 | −2.677 | 4.1E−08 |
| RBP4 | retinol binding protein 4_ plasma | −6.39 | −2.675 | 1.6E−06 |
| CDH1 | cadherin 1_ type 1 | −6.37 | −2.671 | 3.0E−05 |
| COL14A1 | collagen_ type XIV_ alpha 1 | −6.37 | −2.671 | 6.3E−05 |
| SNCG | synuclein_ gamma (breast cancer- | −6.36 | −2.669 | 2.5E−08 |
| | specific protein 1) | | | |
| TSPAN2 | tetraspanin 2 | −6.35 | −2.667 | 4.0E−13 |
| PSG7 | pregnancy specific beta-1-glycoprotein | −6.35 | −2.667 | 3.9E−03 |
| | 7 (gene/pseudogene) | | | |
| LINC00161 | long intergenic non-protein coding | −6.34 | −2.664 | 4.4E−03 |
| | RNA 161 | | | |
| ANXA8L1 | annexin A8-like 1 | −6.33 | −2.662 | 5.9E−07 |
| FAM129A | family with sequence similarity 129_{—} | −6.31 | −2.658 | 3.5E−51 |
| | member A | | | |
| GPR1 | G protein-coupled receptor 1 | −6.30 | −2.656 | 5.4E−55 |
| TEX36 | testis expressed 36 | −6.28 | −2.650 | 4.5E−03 |
| CCL20 | chemokine (C-C motif) ligand 20 | −6.28 | −2.650 | 1.6E−03 |
| LOC101929234 | uncharacterized LOC101929234 | −6.27 | −2.648 | 2.3E−03 |
| ANXA8 | annexin A8 | −6.25 | −2.644 | 7.9E−08 |
| ANO1 | anoctamin 1_ calcium activated | −6.24 | −2.641 | 3.2E−05 |
| | chloride channel | | | |
| MFSD6 | major facilitator superfamily domain | −6.22 | −2.638 | 1.6E−28 |
| | containing 6 | | | |
| LOC101929369 | NA | −6.22 | −2.637 | 7.4E−08 |
| ARHGEF35 | Rho guanine nucleotide exchange | −6.21 | −2.635 | 3.3E−19 |
| | factor (GEF) 35 | | | |
| GPAM | glycerol-3-phosphate acyltransferase_{—} | −6.21 | −2.634 | 6.0E−09 |
| | mitochondrial | | | |
| PRSS35 | protease_ serine_ 35 | −6.20 | −2.632 | 7.7E−07 |
| IFI44 | interferon-induced protein 44 | −6.19 | −2.630 | 1.1E−22 |
| TACR1 | tachykinin receptor 1 | −6.18 | −2.627 | 4.8E−03 |
| COL16A1 | collagen_ type XVI_ alpha 1 | −6.17 | −2.624 | 1.7E−108 |
| FAIM2 | Fas apoptotic inhibitory molecule 2 | −6.16 | −2.623 | 2.6E−07 |
| TULP2 | tubby like protein 2 | −6.15 | −2.621 | 1.6E−03 |
| HERC3 | HECT and RLD domain containing E3 | −6.14 | −2.618 | 1.5E−34 |
| | ubiquitin protein ligase 3 | | | |
| SLC47A1 | solute carrier family 47 (multidrug and | −6.12 | −2.614 | 4.4E−05 |
| | toxin extrusion)_ member 1 | | | |
| SLC30A3 | solute carrier family 30 (zinc | −6.11 | −2.612 | 1.7E−03 |
| | transporter)_ member 3 | | | |
| LOX | lysyl oxidase | −6.10 | −2.609 | 1.2E−104 |
| ACE | angiotensin I converting enzyme | −6.10 | −2.608 | 1.7E−15 |
| PPP4R4 | protein phosphatase 4_ regulatory | −6.09 | −2.605 | 1.1E−03 |
| | subunit 4 | | | |
| RDH5 | retinol dehydrogenase 5 (11-cis/9-cis) | −6.08 | −2.604 | 1.3E−05 |
| CTD- | BEAN1 antisense RNA 1 | −6.08 | −2.604 | 5.1E−03 |
| 2258A20.5 | | | | |
| OTOF | otoferlin | −6.08 | −2.604 | 1.1E−03 |
| ZFP42 | ZFP42 zinc finger protein | −6.08 | −2.603 | 1.8E−04 |
| PCSK6 | proprotein convertase subtilisin/kexin | −6.07 | −2.601 | 3.4E−08 |
| | type 6 | | | |
| FAM13A-AS1 | FAM13A antisense RNA 1 | −6.06 | −2.600 | 7.2E−06 |
| HS3ST3A1 | heparan sulfate (glucosamine) 3-O- | −6.06 | −2.599 | 3.0E−17 |
| | sulfotransferase 3A1 | | | |
| PRKG2 | protein kinase_ cGMP-dependent_{—} | −6.06 | −2.598 | 3.3E−03 |
| | type II | | | |
| KCNT2 | potassium channel_ sodium activated | −6.05 | −2.597 | 1.4E−07 |
| | subfamily T_ member 2 | | | |
| PAMR1 | peptidase domain containing | −6.05 | −2.596 | 2.5E−09 |
| | associated with muscle regeneration 1 | | | |
| MEG3 | maternally expressed 3 (non-protein | −6.03 | −2.593 | 1.5E−24 |
| | coding) | | | |
| NFIX | nuclear factor I/X (CCAAT-binding | −6.03 | −2.591 | 1.6E−91 |
| | transcription factor) | | | |
| EPHA3 | EPH receptor A3 | −6.02 | −2.590 | 5.2E−04 |
| MAP3K8 | mitogen-activated protein kinase | −6.02 | −2.590 | 6.3E−21 |
| | kinase kinase 8 | | | |
| LINC01204 | long intergenic non-protein coding | −6.00 | −2.585 | 9.8E−22 |
| | RNA 1204 | | | |
| PTGIR | prostaglandin I2 (prostacyclin) | −6.00 | −2.584 | 1.7E−93 |
| | receptor (IP) | | | |
| LOR | loricrin | −5.99 | −2.582 | 5.9E−03 |
| NTNG1 | netrin G1 | −5.98 | −2.580 | 1.1E−05 |
| LMO7DN | LMO7 downstream neighbor | −5.98 | −2.579 | 2.1E−03 |
| UNC13A | unc-13 homolog A (C. elegans) | −5.97 | −2.579 | 1.6E−08 |
| FREM1 | FRAS1 related extracellular matrix 1 | −5.97 | −2.578 | 3.2E−03 |
| CYP26B1 | cytochrome P450_ family 26_{—} | −5.97 | −2.577 | 9.4E−05 |
| | subfamily B_ polypeptide 1 | | | |
| LRRC38 | leucine rich repeat containing 38 | −5.96 | −2.576 | 6.1E−03 |
| PDPN | podoplanin | −5.95 | −2.574 | 1.3E−03 |
| RECK | reversion-inducing-cysteine-rich | −5.94 | −2.571 | 1.1E−165 |
| | protein with kazal motifs | | | |
| UNC5B | unc-5 netrin receptor B | −5.94 | −2.570 | 1.4E−06 |
| GOLGA8K | golgin A8 family_ member K | −5.93 | −2.569 | 6.0E−03 |
| ADAMTS1 | ADAM metallopeptidase with | −5.93 | −2.568 | 2.0E−06 |
| | thrombospondin type 1 motif_ 1 | | | |
| C3orf55 | PQ loop repeat containing 2-like | −5.92 | −2.566 | 5.6E−27 |
| NR1D1 | nuclear receptor subfamily 1_ group | −5.91 | −2.564 | 1.5E−82 |
| | D_ member 1 | | | |
| HERC2P4 | hect domain and RLD 2 pseudogene 4 | −5.91 | −2.563 | 2.2E−10 |
| ADD2 | adducin 2 (beta) | −5.90 | −2.561 | 6.5E−03 |
| SLC1A3 | solute carrier family 1 (glial high | −5.89 | −2.559 | 5.4E−12 |
| | affinity glutamate transporter)_{—} | | | |
| | member 3 | | | |
| KLHL33 | kelch-like family member 33 | −5.89 | −2.558 | 1.2E−04 |
| ANGPTL4 | angiopoietin-like 4 | −5.89 | −2.558 | 7.5E−09 |
| MILR1 | mast cell immunoglobulin-like | −5.89 | −2.557 | 8.7E−04 |
| | receptor 1 | | | |
| SLC17A9 | solute carrier family 17 (vesicular | −5.88 | −2.556 | 4.0E−34 |
| | nucleotide transporter)_ member 9 | | | |
| HSPB6 | heat shock protein_ alpha-crystallin- | −5.88 | −2.556 | 1.1E−209 |
| | related_ B6 | | | |
| MIR3613 | microRNA 3613 | −5.87 | −2.554 | 6.3E−03 |
| TSIX | TSIX transcript_ XIST antisense RNA | −5.86 | −2.552 | 6.5E−03 |
| P2RX5 | purinergic receptor P2X_ ligand gated | −5.81 | −2.540 | 9.3E−06 |
| | ion channel_ 5 | | | |
| CRIP1 | cysteine-rich protein 1 (intestinal) | −5.81 | −2.540 | 7.2E−122 |
| CALHM3 | calcium homeostasis modulator 3 | −5.81 | −2.537 | 6.7E−03 |
| TIMP4 | TIMP metallopeptidase inhibitor 4 | −5.79 | −2.533 | 4.2E−17 |
| C11orf70 | chromosome 11 open reading frame 70 | −5.77 | −2.528 | 7.6E−23 |
| PDGFRA | platelet-derived growth factor | −5.77 | −2.527 | 3.2E−11 |
| | receptor_ alpha polypeptide | | | |
| TBX4 | T-box 4 | −5.76 | −2.527 | 1.3E−03 |
| SORCS3 | sortilin-related VPS10 domain | −5.75 | −2.524 | 7.3E−03 |
| | containing receptor 3 | | | |
| SPATA41 | spermatogenesis associated 41 (non- | −5.72 | −2.516 | 3.4E−03 |
| | protein coding) | | | |
| LOC101927905 | uncharacterized LOC101927905 | −5.72 | −2.516 | 5.5E−07 |
| ANKRD20A5P | ankyrin repeat domain 20 family_{—} | −5.70 | −2.512 | 2.7E−03 |
| | member A5_ pseudogene | | | |
| IL1R1 | interleukin 1 receptor_ type I | −5.70 | −2.511 | 1.1E−64 |
| LURAP1L | leucine rich adaptor protein 1-like | −5.70 | −2.511 | 4.5E−16 |
| GDNF-AS1 | GDNF antisense RNA 1 (head to head) | −5.69 | −2.509 | 2.5E−18 |
| LOC100505739 | NA | −5.69 | −2.509 | 7.6E−03 |
| MRO | maestro | −5.68 | −2.506 | 7.6E−03 |
| RAP1GAP | RAP1 GTPase activating protein | −5.68 | −2.505 | 2.6E−05 |
| PDE4C | phosphodiesterase 4C_ cAMP-specific | −5.68 | −2.505 | 7.3E−05 |
| HSD3B7 | hydroxy-delta-5-steroid | −5.67 | −2.504 | 3.0E−57 |
| | dehydrogenase_ 3 beta- and steroid | | | |
| | delta-isomerase 7 | | | |
| PRLR | prolactin receptor | −5.66 | −2.501 | 3.2E−03 |
| ADAMTSL2 | ADAMTS-like 2 | −5.66 | −2.500 | 2.3E−03 |
| SLC38A5 | solute carrier family 38_ member 5 | −5.66 | −2.500 | 3.7E−60 |
| C4BPA | complement component 4 binding | −5.65 | −2.499 | 7.9E−03 |
| | protein_ alpha | | | |
| SLC38A4 | solute carrier family 38_ member 4 | −5.65 | −2.498 | 2.4E−27 |
| MESP2 | mesoderm posterior bHLH | −5.64 | −2.496 | 6.0E−05 |
| | transcription factor 2 | | | |
| LINC01268 | long intergenic non-protein coding | −5.63 | −2.494 | 4.5E−03 |
| | RNA 1268 | | | |
| NPR1 | natriuretic peptide receptor 1 | −5.63 | −2.494 | 2.7E−03 |
| COL3A1 | collagen_ type III_ alpha 1 | −5.63 | −2.493 | 4.8E−26 |
| FAM107A | family with sequence similarity 107_{—} | −5.62 | −2.490 | 2.5E−03 |
| | member A | | | |
| FAM149A | family with sequence similarity 149_{—} | −5.61 | −2.489 | 1.6E−26 |
| | member A | | | |
| HCG4B | HLA complex group 4B (non-protein | −5.61 | −2.488 | 4.9E−04 |
| | coding) | | | |
| CHN1 | chimerin 1 | −5.61 | −2.488 | 7.7E−47 |
| TMTC1 | transmembrane and tetratricopeptide | −5.60 | −2.486 | 1.6E−10 |
| | repeat containing 1 | | | |
| NEAT1 | nuclear paraspeckle assembly | −5.59 | −2.484 | 8.4E−11 |
| | transcript 1 (non-protein coding) | | | |
| IGFL3 | IGF-like family member 3 | −5.59 | −2.482 | 8.7E−03 |
| MFAP3L | microfibrillar-associated protein 3-like | −5.58 | −2.480 | 1.9E−93 |
| PTGS2 | prostaglandin-endoperoxide synthase 2 | −5.58 | −2.479 | 2.1E−05 |
| | (prostaglandin G/H synthase and | | | |
| | cyclooxygenase) | | | |
| LINC00312 | long intergenic non-protein coding | −5.56 | −2.476 | 1.5E−04 |
| | RNA 312 | | | |
| PLA2G2A | phospholipase A2_ group IIA | −5.55 | −2.473 | 9.1E−03 |
| | (platelets_ synovial fluid) | | | |
| RGMA | repulsive guidance molecule family | −5.54 | −2.470 | 1.0E−05 |
| | member a | | | |
| CCDC158 | coiled-coil domain containing 158 | −5.53 | −2.468 | 4.2E−03 |
| EMP1 | epithelial membrane protein 1 | −5.53 | −2.468 | 1.3E−33 |
| MT1G | metallothionein 1G | −5.53 | −2.467 | 4.5E−10 |
| ITGB8 | integrin_ beta 8 | −5.53 | −2.467 | 4.5E−10 |
| ZNF311 | zinc finger protein 311 | −5.53 | −2.466 | 2.1E−09 |
| HSD3B1 | hydroxy-delta-5-steroid | −5.50 | −2.458 | 9.6E−03 |
| | dehydrogenase_ 3 beta- and steroid | | | |
| | delta-isomerase 1 | | | |
| F10 | coagulation factor X | −5.48 | −2.454 | 1.1E−08 |
| LINC00478 | mir-99a-let-7c cluster host gene | −5.47 | −2.453 | 5.2E−04 |
| DOK6 | docking protein 6 | −5.46 | −2.450 | 2.5E−08 |
| MIR193A | microRNA 193a | −5.46 | −2.448 | 6.1E−04 |
| RASL11B | RAS-like_ family 11_ member B | −5.45 | −2.446 | 7.0E−05 |
| AKR1C3 | aldo-keto reductase family 1_ member | −5.44 | −2.445 | 5.2E−11 |
| | C3 | | | |
| FAS | Fas cell surface death receptor | −5.44 | −2.444 | 1.8E−61 |
| KY | kyphoscoliosis peptidase | −5.44 | −2.443 | 3.2E−15 |
| AGAP11 | ankyrin repeat and GTPase domain Arf | −5.43 | −2.442 | 9.8E−06 |
| | GTPase activating protein 11 | | | |
| PRRG4 | proline rich Gla (G-carboxyglutamic | −5.43 | −2.441 | 2.1E−04 |
| | acid) 4 (transmembrane) | | | |
| MUC12 | mucin 12_ cell surface associated | −5.43 | −2.441 | 6.6E−03 |
| CYP21A2 | cytochrome P450_ family 21_{—} | −5.42 | −2.439 | 1.0E−02 |
| | subfamily A_ polypeptide 2 | | | |
| IL6 | interleukin 6 | −5.41 | −2.436 | 1.2E−05 |
| ANKRD29 | ankyrin repeat domain 29 | −5.40 | −2.433 | 1.7E−05 |
| NFIA | nuclear factor I/A | −5.39 | −2.431 | 8.0E−25 |
| TENM2 | teneurin transmembrane protein 2 | −5.39 | −2.430 | 1.3E−03 |
| LOC101928200 | NA | −5.38 | −2.427 | 3.0E−05 |
| CAMK2B | calcium/calmodulin-dependent protein | −5.37 | −2.426 | 8.0E−03 |
| | kinase II beta | | | |
| CDC20B | cell division cycle 20B | −5.35 | −2.420 | 1.1E−02 |
| FPR3 | formyl peptide receptor 3 | −5.35 | −2.419 | 1.1E−02 |
| MIR10A | microRNA 10a | −5.33 | −2.415 | 1.1E−02 |
| TTC3P1 | tetratricopeptide repeat domain 3 | −5.33 | −2.414 | 4.5E−08 |
| | pseudogene 1 | | | |
| LY86 | lymphocyte antigen 86 | −5.33 | −2.414 | 1.1E−02 |
| HRNR | hornerin | −5.32 | −2.412 | 1.1E−02 |
| SERPING1 | serpin peptidase inhibitor_ clade G (C1 | −5.31 | −2.409 | 1.4E−15 |
| | inhibitor)_ member 1 | | | |
| NRG1 | neuregulin 1 | −5.31 | −2.409 | 7.4E−09 |
| ALDH1A3 | aldehyde dehydrogenase 1 family_{—} | −5.31 | −2.408 | 3.9E−05 |
| | member A3 | | | |
| IL20RB | interleukin 20 receptor beta | −5.30 | −2.407 | 1.7E−11 |
| MMP10 | matrix metallopeptidase 10 | −5.30 | −2.406 | 5.2E−05 |
| ZNF704 | zinc finger protein 704 | −5.29 | −2.403 | 1.2E−04 |
| OR2S2 | olfactory receptor_ family 2_{—} | −5.29 | −2.403 | 2.4E−03 |
| | subfamily S_ member 2 | | | |
| | (gene/pseudogene) | | | |
| RSPO3 | R-spondin 3 | −5.27 | −2.398 | 7.5E−04 |
| BEND7 | BEN domain containing 7 | −5.27 | −2.397 | 9.5E−22 |
| C21orf90 | TSPEAR antisense RNA 2 | −5.26 | −2.396 | 1.2E−02 |
| SBSPON | somatomedin B and thrombospondin_{—} | −5.26 | −2.395 | 9.7E−04 |
| | type 1 domain containing | | | |
| EEF1DP3 | eukaryotic translation elongation factor | −5.26 | −2.395 | 3.2E−04 |
| | 1 delta pseudogene 3 | | | |
| LY6K | lymphocyte antigen 6 complex_ locus | −5.25 | −2.392 | 5.2E−03 |
| | K | | | |
| ENPP4 | ectonucleotide | −5.25 | −2.392 | 5.9E−10 |
| | pyrophosphatase/phosphodiesterase 4_{—} | | | |
| | (putative) | | | |
| EVPL | envoplakin | −5.24 | −2.390 | 9.7E−03 |
| SFN | stratifin | −5.23 | −2.386 | 8.8E−03 |
| CYP4V2 | cytochrome P450_ family 4 | −5.22 | −2.385 | 4.0E−15 |
| | subfamily V_ polypeptide 2 | | | |
| GJB5 | gap junction protein beta 5 31.1 kDa | −5.22 | −2.384 | 1.2E−02 |
| SERPINB2 | serpin peptidase inhibitor_ clade B | −5.21 | −2.382 | 5.0E−05 |
| | (ovalbumin)_ member 2 | | | |
| C2 | complement component 2 | −5.21 | −2.382 | 2.0E−06 |
| LMO2 | LIM domain only 2 (rhombotin-like 1) | −5.21 | −2.381 | 5.2E−05 |
| ELTD1 | adhesion G protein-coupled receptor | −5.20 | −2.379 | 1.3E−48 |
| | L4 | | | |
| ESR1 | estrogen receptor 1 | −5.20 | −2.378 | 1.3E−02 |
| MYH8 | myosin_ heavy chain 8_ skeletal | −5.20 | −2.378 | 1.2E−02 |
| | muscle_ perinatal | | | |
| GDNF | glial cell derived neurotrophic factor | −5.19 | −2.376 | 9.7E−59 |
| KRT222 | keratin 222_ type II | −5.18 | −2.374 | 1.2E−02 |
| SNTB1 | syntrophin_ beta 1 (dystrophin- | −5.18 | −2.372 | 1.0E−05 |
| | associated protein A1_ 59 kDa_ basic | | | |
| | component 1) | | | |
| PRUNE2 | prune homolog 2 (Drosophila) | −5.17 | −2.371 | 5.6E−03 |
| PCDHGA5 | protocadherin gamma subfamily A_ 5 | −5.16 | −2.368 | 1.2E−05 |
| LBX2 | ladybird homeobox 2 | −5.16 | −2.367 | 2.8E−09 |
| LINC01119 | long intergenic non-protein coding | −5.16 | −2.367 | 2.6E−11 |
| | RNA 1119 | | | |
| SLC4A4 | solute carrier family 4 (sodium | −5.15 | −2.365 | 4.2E−83 |
| | bicarbonate cotransporter)_ member 4 | | | |
| SEL1L3 | sel-1 suppressor of lin-12-like 3 (C. | −5.15 | −2.364 | 1.7E−90 |
| | elegans) | | | |
| HSPA7 | heat shock 70 kDa protein 7 (HSP70B) | −5.14 | −2.363 | 3.1E−04 |
| PRKD1 | protein kinase D1 | −5.13 | −2.358 | 2.6E−50 |
| ADPRH | ADP-ribosylarginine hydrolase | −5.13 | −2.358 | 5.5E−30 |
| GPR116 | adhesion G protein-coupled receptor | −5.11 | −2.354 | 1.4E−02 |
| | F5 | | | |
| NKPD1 | NTPase_ KAP family P-loop domain | −5.11 | −2.354 | 7.5E−03 |
| | containing 1 | | | |
| CNTD2 | cyclin N-terminal domain containing 2 | −5.11 | −2.354 | 1.1E−04 |
| GAL | galanin/GMAP prepropeptide | −5.10 | −2.351 | 7.0E−16 |
| ENPP1 | ectonucleotide | −5.09 | −2.349 | 6.2E−07 |
| | pyrophosphatase/phosphodiesterase 1 | | | |
| SERINC2 | serine incorporator 2 | −5.09 | −2.347 | 4.9E−18 |
| ASS1 | argininosuccinate synthase 1 | −5.08 | −2.344 | 5.7E−09 |
| PITX2 | paired-like homeodomain 2 | −5.07 | −2.343 | 7.4E−05 |
| LINC00933 | long intergenic non-protein coding | −5.07 | −2.342 | 4.4E−03 |
| | RNA 933 | | | |
| C11orf96 | chromosome 11 open reading frame 96 | −5.06 | −2.340 | 2.3E−03 |
| APOBEC3G | apolipoprotein B mRNA editing | −5.04 | −2.332 | 7.4E−21 |
| | enzyme_ catalytic polypeptide-like 3G | | | |
| MBP | myelin basic protein | −5.02 | −2.329 | 5.9E−14 |
| RGS7BP | regulator of G-protein signaling 7 | −5.02 | −2.329 | 1.5E−02 |
| | binding protein | | | |
| ACKR4 | atypical chemokine receptor 4 | −5.02 | −2.327 | 7.8E−05 |
| TYMP | thymidine phosphorylase | −5.01 | −2.324 | 2.4E−31 |
| MAB21L3 | mab-21-like 3 (C. elegans) | −5.01 | −2.324 | 1.5E−02 |
| DENND2C | DENN/MADD domain containing 2C | −5.00 | −2.323 | 2.7E−07 |
| FLJ46906 | uncharacterized LOC441172 | −5.00 | −2.321 | 6.7E−21 |
| PSG11 | pregnancy specific beta-1-glycoprotein | −5.00 | −2.321 | 1.5E−02 |
| | 11 | | | |

### Example 12. miRNA Nanostring nCounter Analysis of HMC-EVs Vs BM-MSC-EVs Vs UCB-MSC-EVs Vs AD-MSC-EVs

HMCs were generated from the same bank of frozen hemangioblasts described in Example 1. HMCs were generated and passaged up to six passages (P6) according to the method described in Example 1. Extracellular vesicles (EVs) were purified from HMCs (HMC-EVs) by tangential flow filtration (TFF). miRNA profiling was performed using Nanostring nCounter Analysis system for three lots of HMC-EVs under basal conditions. EVs isolated from bone marrow (BM-MSC-EVs) (3 lots), umbilical cord blood (UCB-MSC-EVs) (3 lots), and adipose tissue (AD-MSC-EVs) under basal conditions were used as controls.

Table 9 shows miRNAs that were more highly expressed in the HMC-EVs compared with UCB-MSC-EVs. Table 10 shows miRNAs that were more highly expressed in UCB-MSC-EVs compared with the HMC-EVs. Table 11 shows miRNAs that were highly expressed in HMC-EVs compared with BM-MSC-EVs. Table 12 shows miRNAs that were more highly expressed in BM-MSC-EVs compared with the HMC-EVs. Table 13 shows miRNAs that were highly expressed in HMC-EVs compared with AD-MSC-EVs. Table 14 shows miRNAs that were more highly expressed in AD-MSC-EVs compared with the HMC-EVs. HMC-EVs of the presently disclosed subject matter may be selected or purified based on an of the miRNAs that are differentially expressed.

TABLE 9
miRNAs with higher expression in
HMC-EVs compared to UCB-MSC-EVs

| miRNA ID | Fold Difference | p-Value |
|---|---|---|
| hsa-miR-125b-5p | 3.90 | 0.000 |
| hsa-miR-100-5p | 3.50 | 0.004 |
| hsa-miR-21-5p | 2.59 | 0.025 |
| hsa-miR-199a-3p + hsa-miR-199b-3p | 2.57 | 0.000 |
| hsa-miR-23a-3p | 2.37 | 0.013 |
| hsa-miR-181a-5p | 2.16 | 0.007 |
| hsa-miR-199b-5p | 2.07 | 0.000 |
| hsa-miR-125a-5p | 2.05 | 0.008 |
| hsa-miR-1204 | 1.96 | 0.035 |
| hsa-miR-106a-5p + hsa-miR-17-5p | 1.73 | 0.013 |
| hsa-let-7e-5p | 1.68 | 0.017 |
| hsa-miR-450a-5p | 1.67 | 0.014 |

TABLE 10
miRNAs with higher expression in
UCB- MSC-EVs compared to HMC-EVs

| miRNA ID | Fold Difference | p-Value |
|---|---|---|
| hsa-miR-1252-5p | −2.04 | 0.00 |
| hsa-miR-376c-3p | −2.00 | 0.01 |
| hsa-miR-196b-5p | −1.93 | 0.02 |
| hsa-miR-4755-5p | −1.83 | 0.00 |
| hsa-miR-211-3p | −1.81 | 0.05 |
| hsa-miR-548d-3p | −1.66 | 0.05 |
| hsa-miR-671-3p | −1.66 | 0.03 |
| hsa-miR-1297 | −1.56 | 0.01 |
| hsa-miR-134-5p + hsa-miR-6728-5p | −1.55 | 0.05 |
| hsa-mir-498 | −1.52 | 0.01 |
| hsa-miR-128-1-5p | −1.52 | 0.01 |
| hsa-miR-1269b | −1.51 | 0.01 |

TABLE 11
miRNAs with higher expression in
HMC-EVs compared to BM-MSC-EVs

| miRNA ID | Fold Difference | p-Value |
|---|---|---|
| hsa-miR-320e | 13.68 | 0.035 |
| hsa-miR-125b-5p | 4.81 | 0.000 |
| hsa-miR-100-5p | 4.38 | 0.001 |
| hsa-miR-181a-5p | 3.42 | 0.007 |
| hsa-miR-23a-3p | 3.03 | 0.006 |
| hsa-miR-21-5p | 2.95 | 0.012 |
| hsa-miR-199a-3p + hsa-miR-199b-3p | 2.86 | 0.007 |
| hsa-let-7a-5p | 2.30 | 0.032 |
| hsa-miR-221-3p | 2.18 | 0.005 |
| hsa-miR-199b-5p | 2.07 | 0.000 |
| hsa-miR-29a-3p | 1.67 | 0.019 |
| hsa-miR-125a-5p | 1.64 | 0.034 |
| hsa-let-7g-5p | 1.54 | 0.025 |

TABLE 12
miRNAs with higher expression in
BM- MSC-EVs compared to HMC-EVs

| miRNA ID | Fold Difference | p-Value |
|---|---|---|
| hsa-miR-1469 | −2.34 | 0.026 |
| hsa-miR-892b | −2.29 | 0.004 |
| hsa-miR-664b-5p | −2.27 | 0.003 |
| hsa-miR-151b | −2.20 | 0.012 |
| hsa-miR-219a-2-3p | −2.16 | 0.035 |
| hsa-miR-485-3p | −2.14 | 0.010 |
| hsa-miR-134-5p + hsa-miR-6728-5p | −2.07 | 0.008 |
| hsa-miR-195-5p | −2.05 | 0.014 |
| hsa-miR-508-3p | −2.03 | 0.004 |
| hsa-miR-5010-5p | −2.01 | 0.032 |
| hsa-miR-629-5p | −1.99 | 0.018 |
| hsa-miR-518d-3p | −1.99 | 0.035 |
| hsa-miR-18b-5p | −1.98 | 0.037 |
| hsa-miR-147a | −1.92 | 0.048 |
| hsa-miR-196b-5p | −1.90 | 0.013 |
| hsa-miR-486-3p | −1.88 | 0.032 |
| hsa-miR-1258 | −1.85 | 0.023 |
| hsa-miR-548aa + hsa-miR-548t-3p | −1.81 | 0.034 |
| hsa-miR-584-5p | −1.81 | 0.047 |
| hsa-miR-3202 | −1.80 | 0.012 |
| hsa-miR-663a | −1.80 | 0.034 |
| hsa-miR-517a-3p | −1.80 | 0.013 |
| hsa-miR-329-3p | −1.80 | 0.019 |
| hsa-miR-1248 | −1.76 | 0.035 |
| hsa-miR-628-3p | −1.76 | 0.013 |
| hsa-miR-499b-5p | −1.75 | 0.038 |
| hsa-miR-1279 | −1.74 | 0.017 |
| hsa-miR-873-3p | −1.74 | 0.048 |
| hsa-miR-514a-5p | −1.73 | 0.008 |
| hsa-miR-127-5p | −1.72 | 0.048 |
| hsa-miR-491-3p | −1.71 | 0.019 |
| hsa-miR-548k | −1.71 | 0.013 |
| hsa-miR-566 | −1.70 | 0.036 |
| hsa-miR-520c-3p | −1.69 | 0.036 |
| hsa-miR-591 | −1.68 | 0.012 |
| hsa-miR-129-5p | −1.67 | 0.013 |
| hsa-miR-6503-3p | −1.66 | 0.011 |
| hsa-miR-1183 | −1.65 | 0.003 |
| hsa-miR-1178-3p | −1.65 | 0.046 |
| hsa-miR-885-3p | −1.65 | 0.019 |
| hsa-miR-6721-5p | −1.62 | 0.013 |
| hsa-miR-4536-5p | −1.61 | 0.033 |
| hsa-miR-617 | −1.61 | 0.027 |
| hsa-miR-510-5p | −1.59 | 0.031 |
| hsa-mir-498 | −1.59 | 0.017 |
| hsa-miR-142-5p | −1.59 | 0.006 |
| hsa-miR-378d | −1.58 | 0.014 |
| hsa-miR-3131 | −1.58 | 0.016 |
| hsa-miR-578 | −1.57 | 0.041 |
| hsa-miR-450a-2-3p | −1.57 | 0.002 |
| hsa-miR-620 | −1.57 | 0.024 |
| hsa-miR-3613-3p | −1.57 | 0.012 |
| hsa-miR-1234-3p | −1.57 | 0.049 |
| hsa-miR-1269b | −1.57 | 0.029 |
| hsa-miR-940 | −1.56 | 0.007 |
| hsa-miR-4787-5p | −1.55 | 0.019 |
| hsa-miR-378h | −1.55 | 0.005 |
| hsa-miR-654-5p | −1.53 | 0.028 |
| hsa-miR-92b-3p | −1.51 | 0.044 |

TABLE 13
miRNAs with higher expression in
HMC-EVs compared to AD-MSC-EVs

| miRNA ID | Fold Difference | p-Value |
|---|---|---|
| hsa-miR-125b-5p | 5.73 | 0.004 |
| hsa-miR-4454 + hsa-miR-7975 | 4.31 | 0.001 |
| hsa-miR-100-5p | 4.03 | 0.002 |
| hsa-miR-181a-5p | 3.39 | 0.001 |
| hsa-miR-21-5p | 3.20 | 0.021 |
| hsa-miR-199a-3p + hsa-miR-199b-3p | 3.06 | 0.011 |
| hsa-miR-23a-3p | 2.69 | 0.007 |
| hsa-miR-125a-5p | 2.22 | 0.024 |
| hsa-miR-29a-3p | 2.14 | 0.024 |
| hsa-miR-450a-5p | 2.11 | 0.004 |
| hsa-miR-25-3p | 2.02 | 0.000 |
| hsa-miR-221-3p | 1.99 | 0.009 |
| hsa-miR-106a-5p + hsa-miR-17-5p | 1.79 | 0.001 |
| hsa-miR-199b-5p | 1.76 | 0.027 |
| hsa-miR-214-3p | 1.65 | 0.034 |

TABLE 14
miRNAs with higher expression in
AD-MSC EVs compared to HMC-EVs

| miRNA ID | Fold Difference | p-Value |
|---|---|---|
| hsa-miR-194-5p | −2.54 | 0.023 |
| hsa-miR-665 | −2.05 | 0.025 |
| hsa-miR-219a-2-3p | −1.95 | 0.046 |
| hsa-miR-4536-3p | −1.91 | 0.049 |
| hsa-miR-18b-5p | −1.87 | 0.039 |
| hsa-miR-124-3p | −1.83 | 0.042 |
| hsa-miR-127-5p | −1.83 | 0.016 |
| hsa-miR-628-3p | −1.83 | 0.026 |
| hsa-miR-2110 | −1.80 | 0.022 |
| hsa-miR-566 | −1.77 | 0.027 |
| hsa-miR-4755-5p | −1.76 | 0.025 |
| hsa-miR-509-3p | −1.76 | 0.003 |
| hsa-miR-578 | −1.71 | 0.029 |
| hsa-miR-1248 | −1.66 | 0.030 |
| hsa-miR-1252-5p | −1.63 | 0.034 |
| hsa-miR-28-5p | −1.63 | 0.005 |
| hsa-miR-128-1-5p | −1.62 | 0.014 |
| hsa-miR-1183 | −1.62 | 0.004 |
| hsa-miR-1296-3p | −1.61 | 0.045 |
| hsa-miR-1285-5p | −1.61 | 0.015 |
| hsa-miR-485-3p | −1.60 | 0.032 |
| hsa-miR-514a-5p | −1.59 | 0.039 |
| hsa-mir-498 | −1.58 | 0.024 |
| hsa-miR-330-5p | −1.56 | 0.020 |
| hsa-miR-10a-5p | −1.55 | 0.038 |
| hsa-miR-888-5p | −1.55 | 0.013 |
| hsa-miR-183-5p | −1.52 | 0.049 |
| hsa-miR-760 | −1.51 | 0.016 |
| hsa-miR-6721-5p | −1.51 | 0.019 |
| hsa-miR-664b-5p | −1.50 | 0.025 |

### Example 13. Proteome Profiling for HMC-EVs Vs BM-MSC-EVs Vs UCB-MSC-EVs Vs AD-MSC-EVs

HMCs were generated from the same bank of frozen hemangioblasts described in Example 1. HMCs were generated and passaged up to six passages (P6) according to the method described in Example 1. Extracellular vesicles (EVs) were purified from HMCs (HMC-EVs) by tangential flow filtration (TFF). Proteome profiling by standard mass spectrometry analysis was performed for three lots of HMC-EVs under basal conditions. EVs isolated from bone marrow (BM-MSC-EVs) (3 lots), umbilical cord blood (UCB-MSC-EVs) (3 lots), and adipose tissue (AD-MSC-EVs) under basal conditions were used as controls.

T-test statistical analysis was used to identify proteins with significant differences in abundance between EV types. Table 15 shows proteins that were more highly abundant in the HMC-EVs compared with UCB-MSC-EVs. Table 16 shows proteins that were more highly abundant in UCB-MSC-EVs compared with the HMC-EVs. Table 17 shows proteins that were more highly abundant in HMC-EVs compared with BM-MSC-EVs. Table 18 shows proteins that were more highly abundant in BM-MSC-EVs compared with the HMC-EVs. Table 19 shows proteins that were more highly abundant in HMC-EVs compared with AD-MSC-EVs. Table 20 shows proteins that were more highly abundant in AD-MSC-EVs compared with the HMC-EVs. HMC-EVs of the presently disclosed subject matter may be selected or purified based on any of the proteins that are differentially abundant.

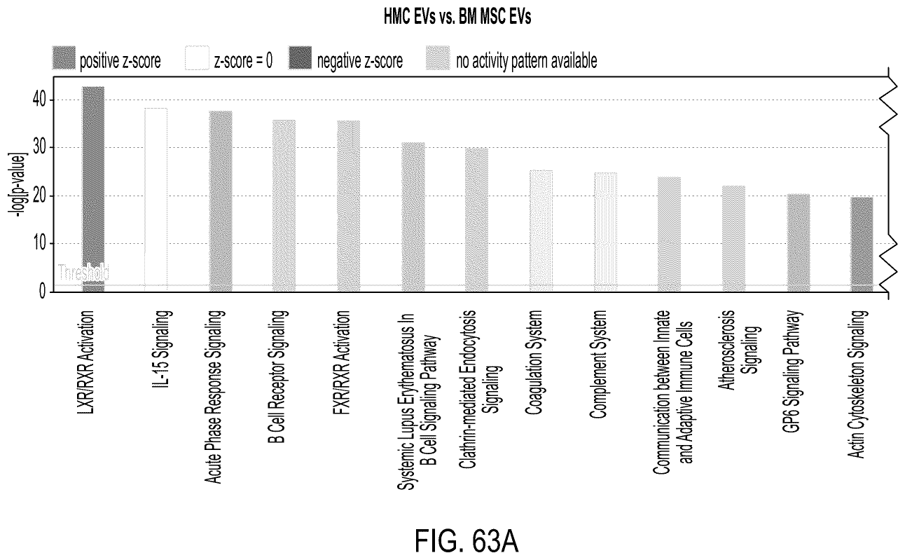

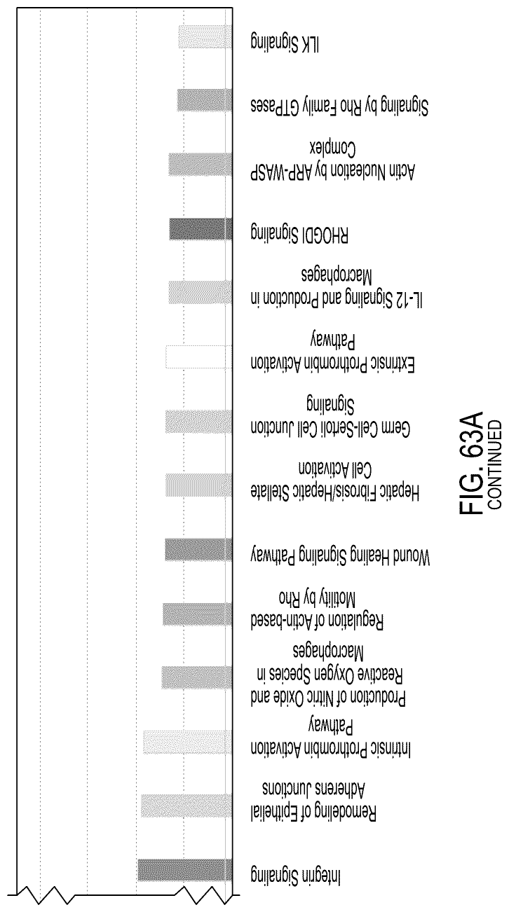

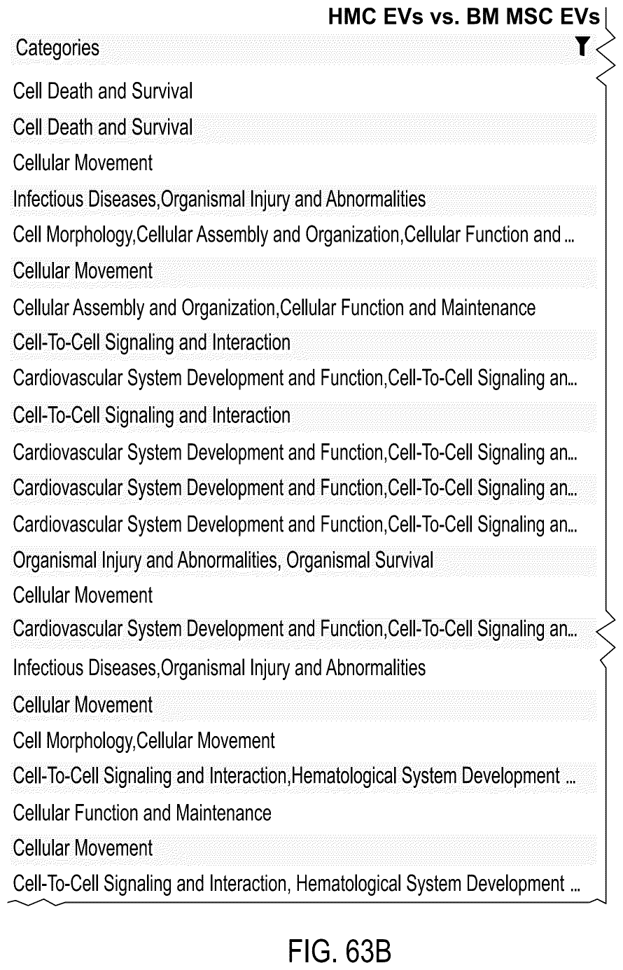

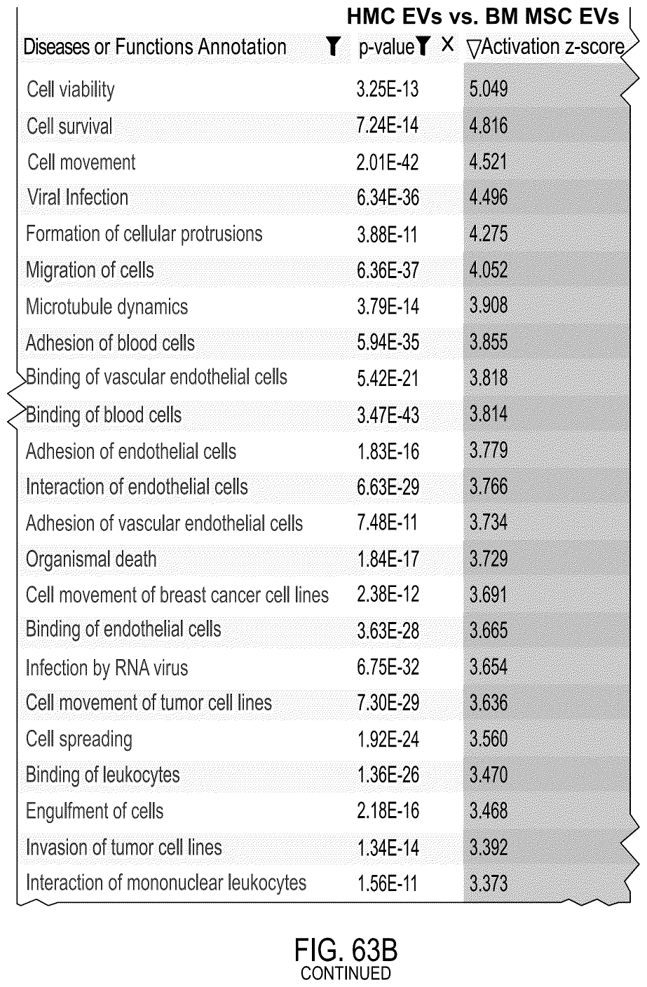

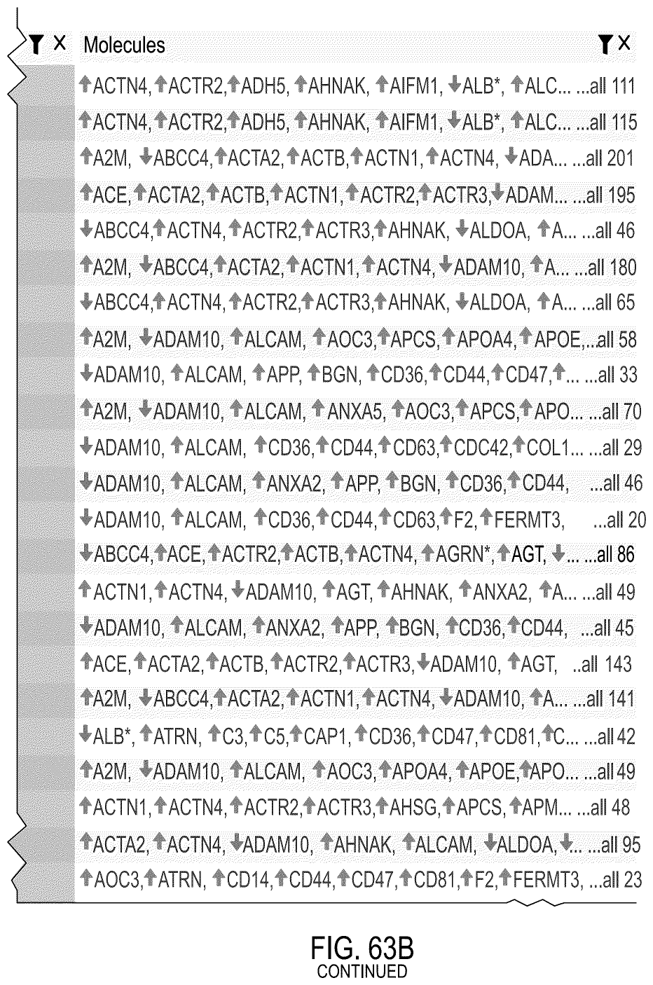

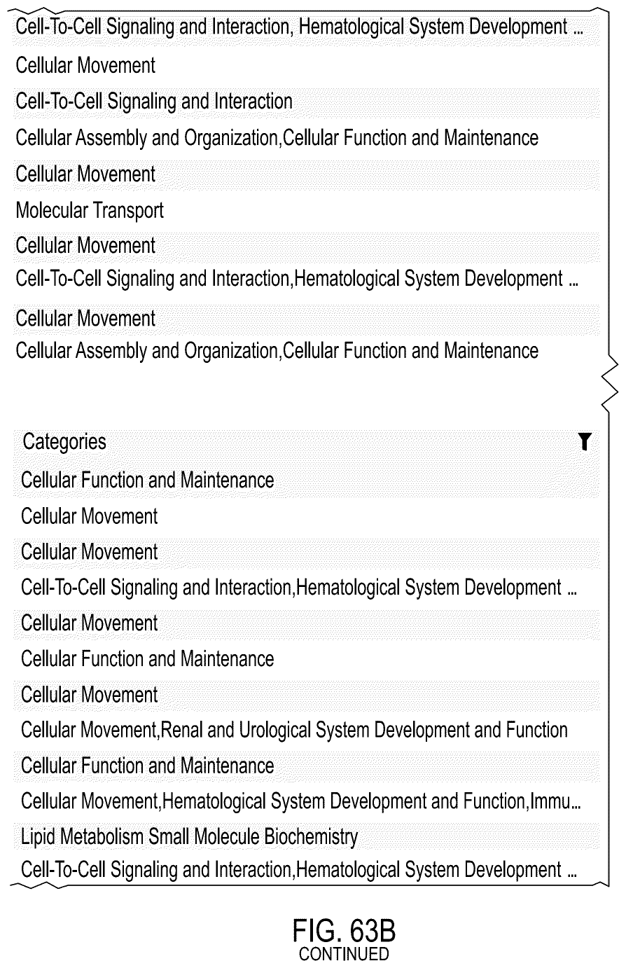

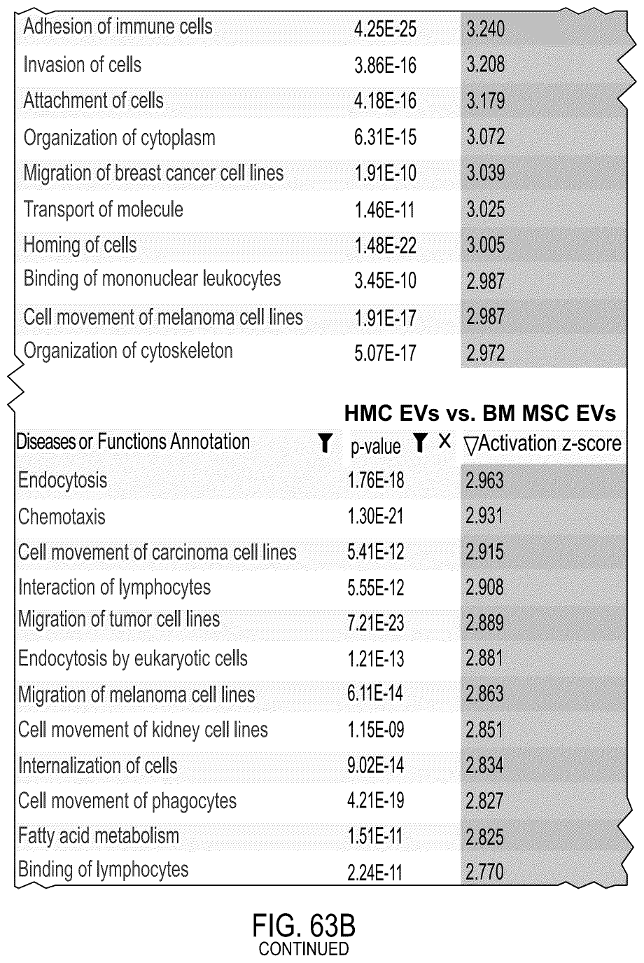

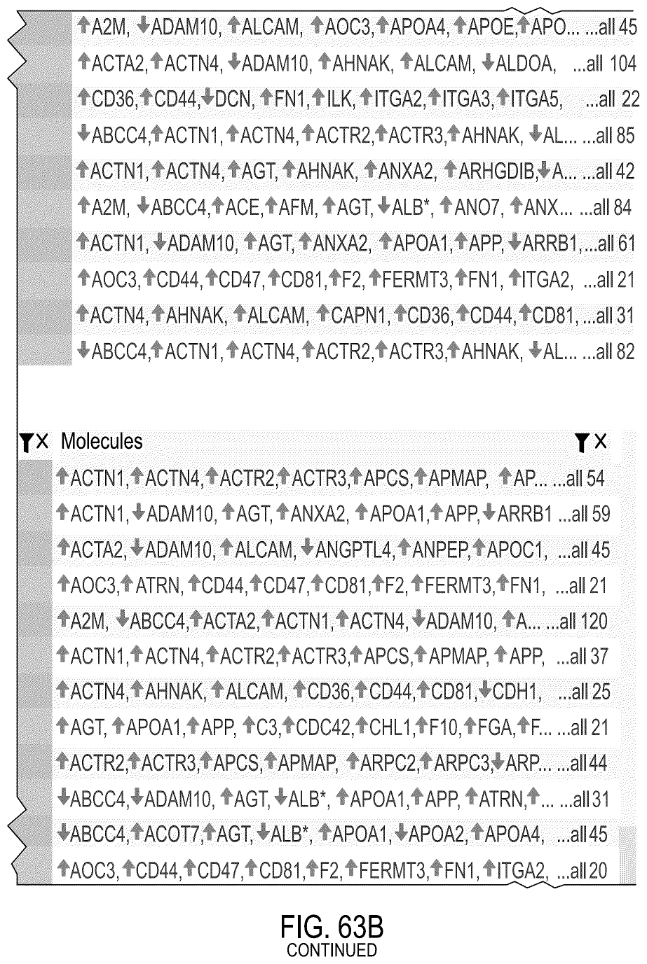

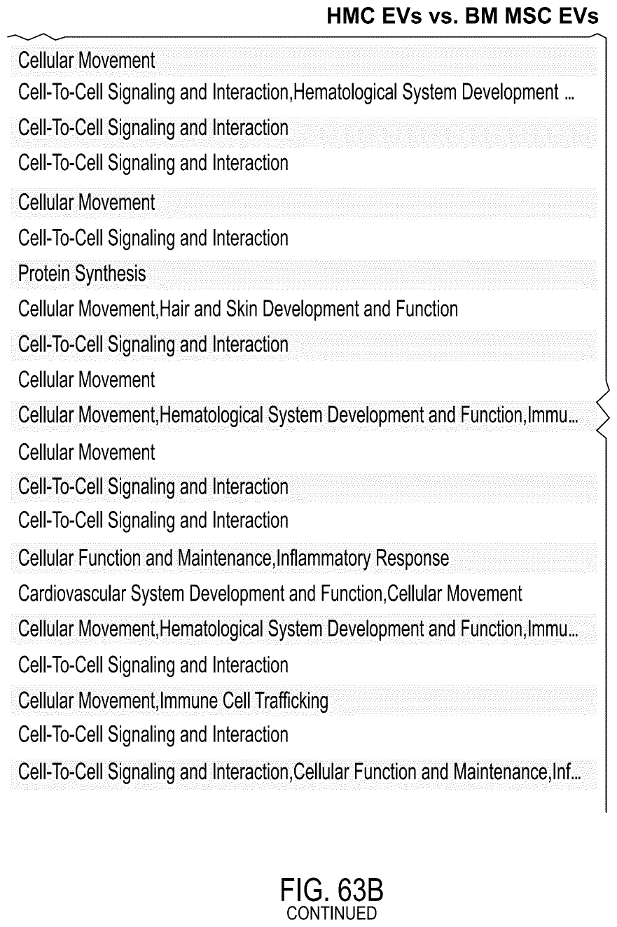

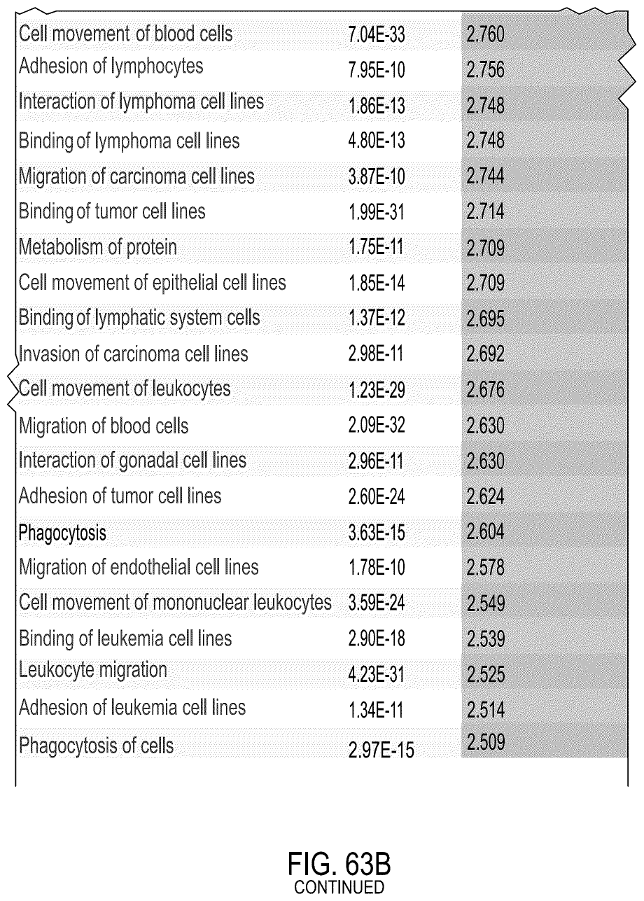

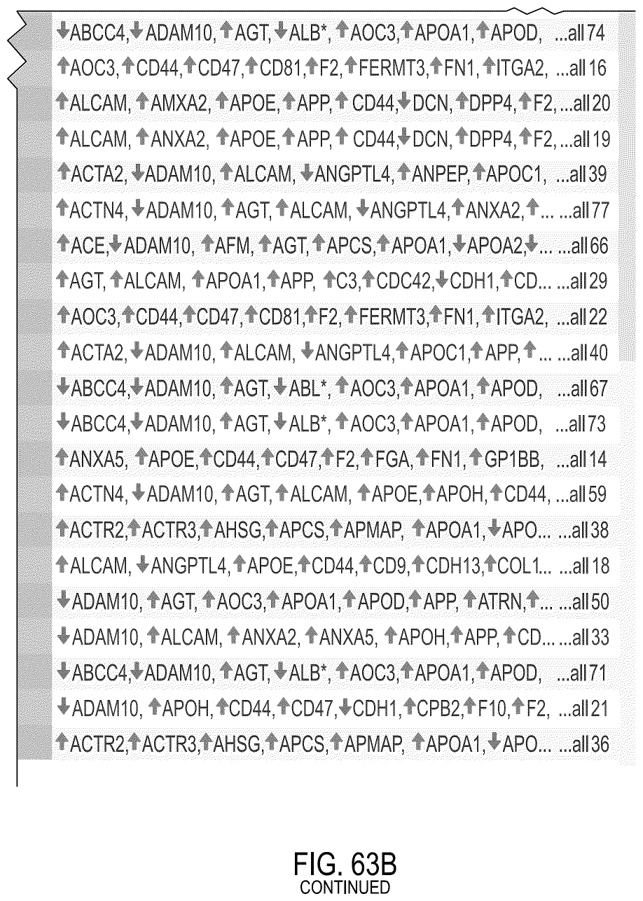

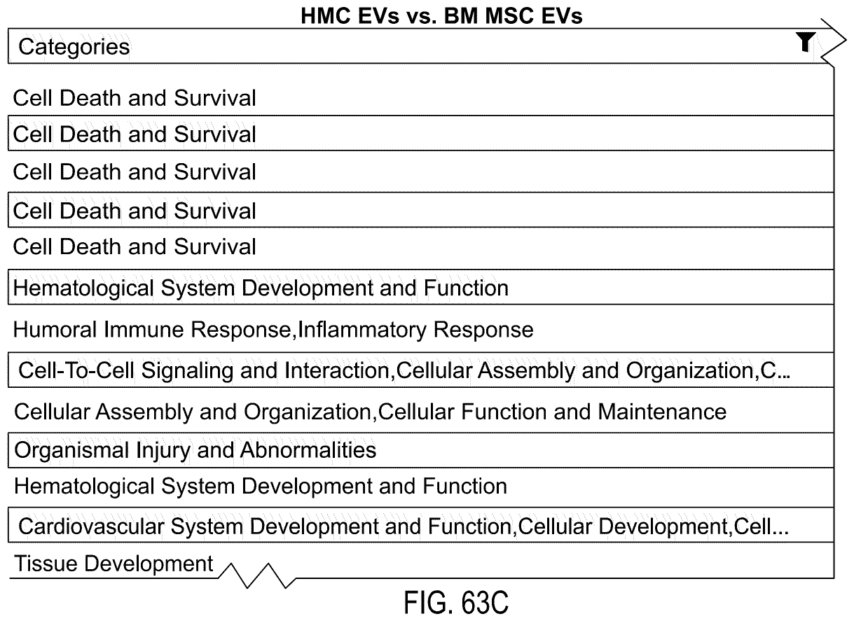

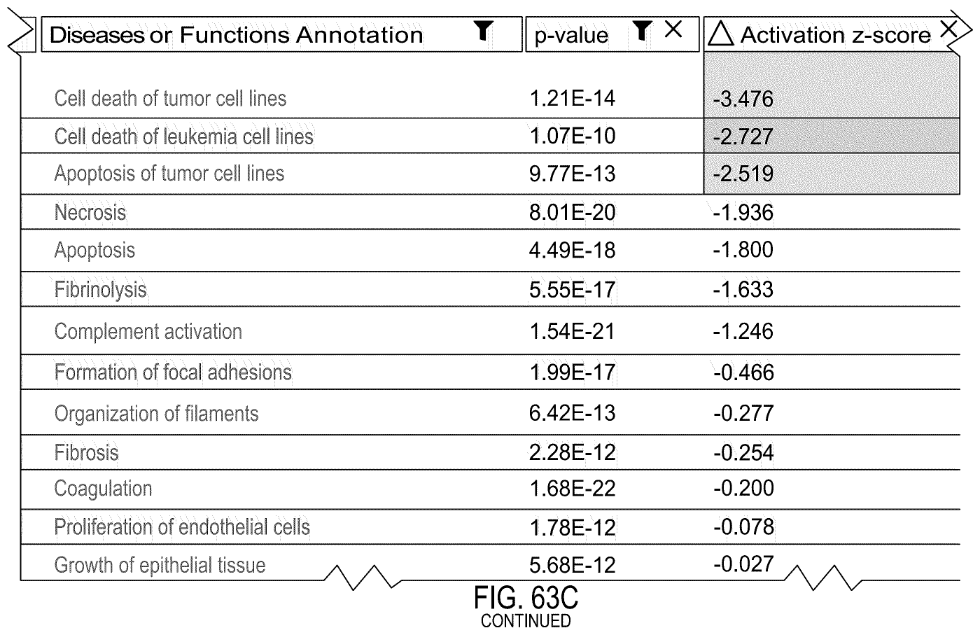

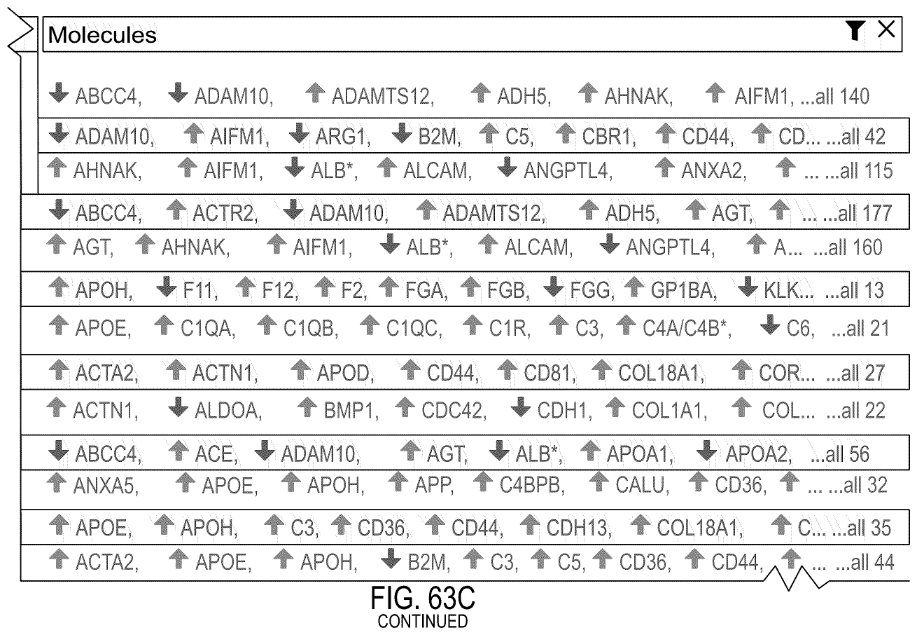

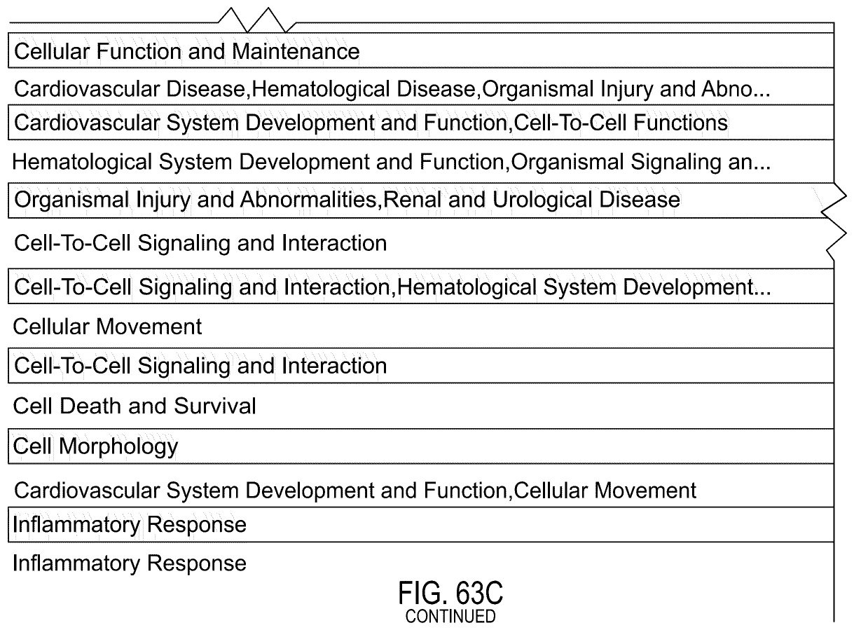

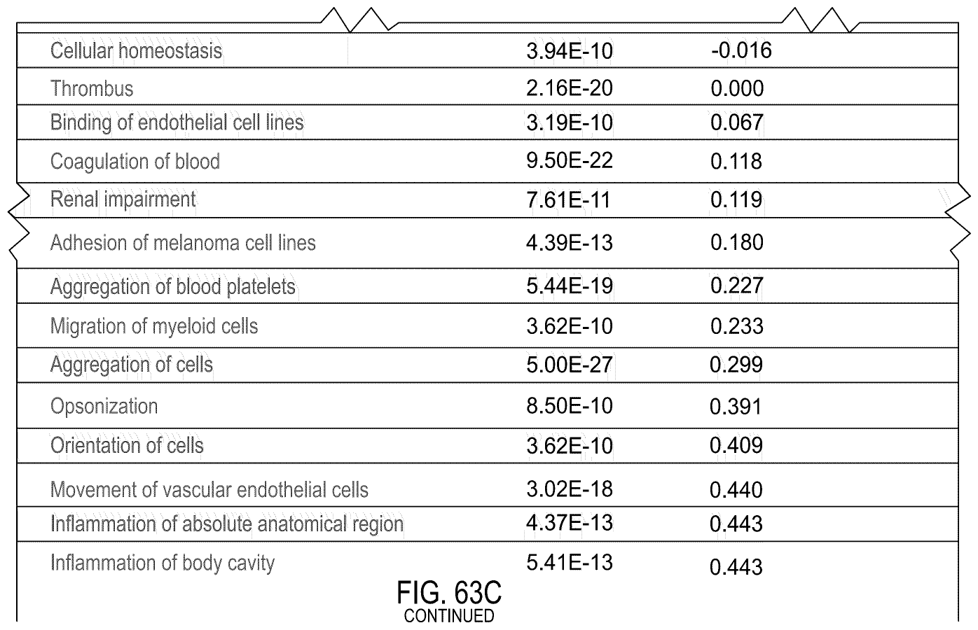

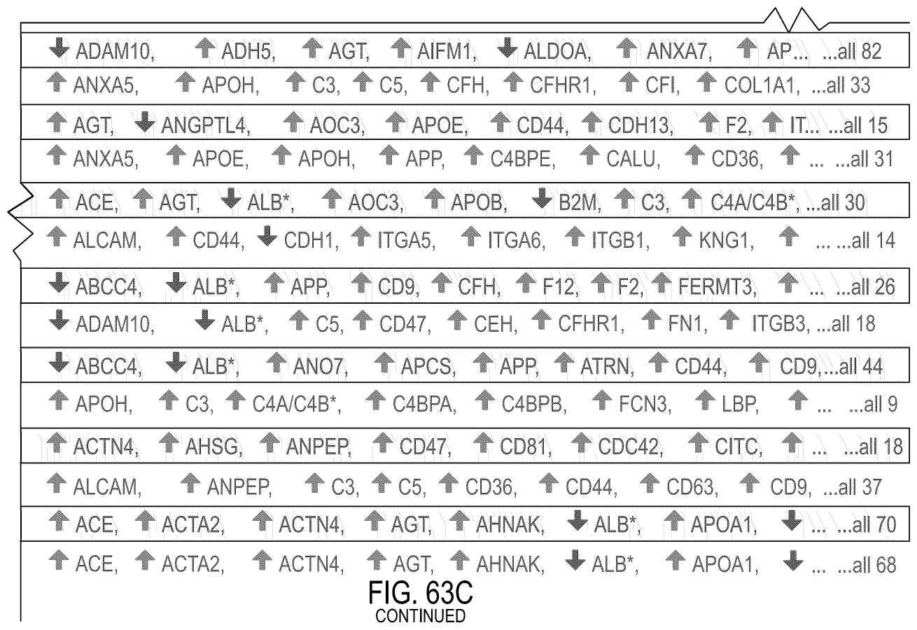

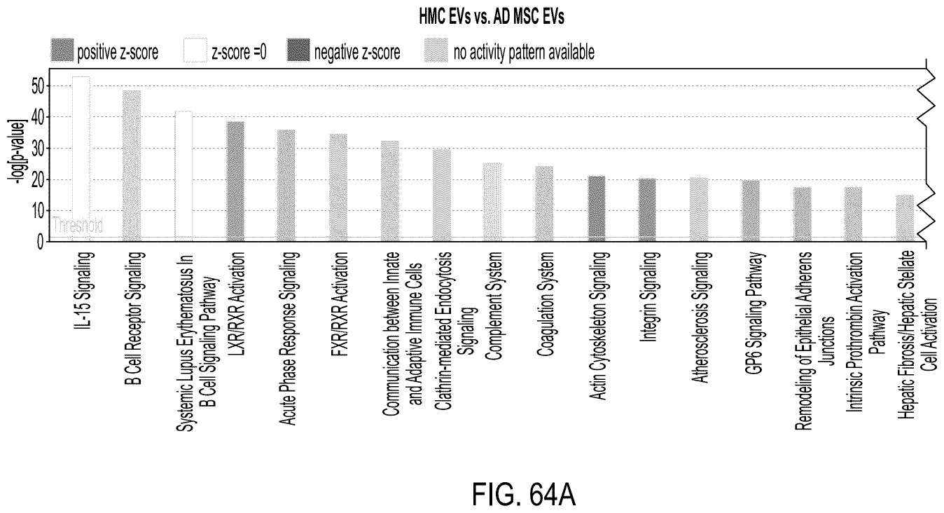

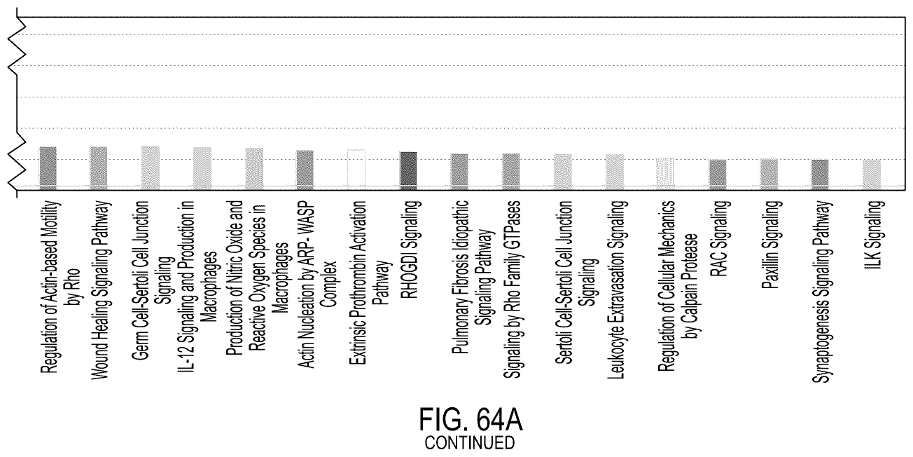

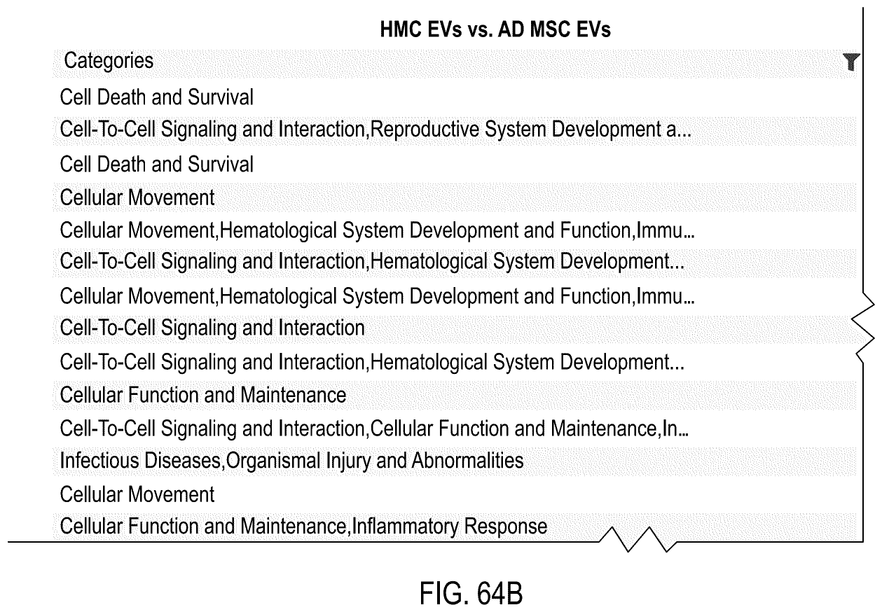

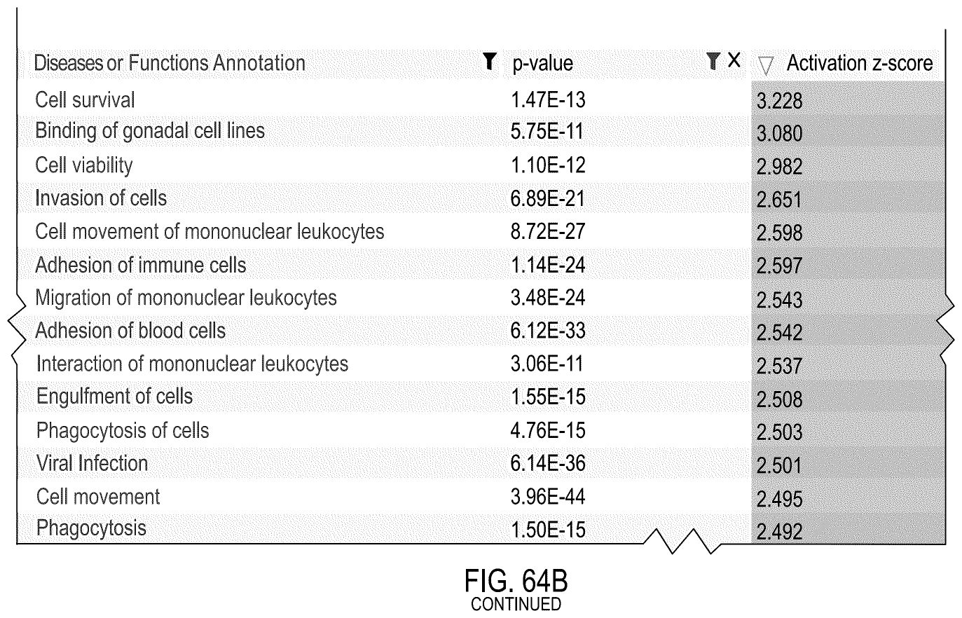

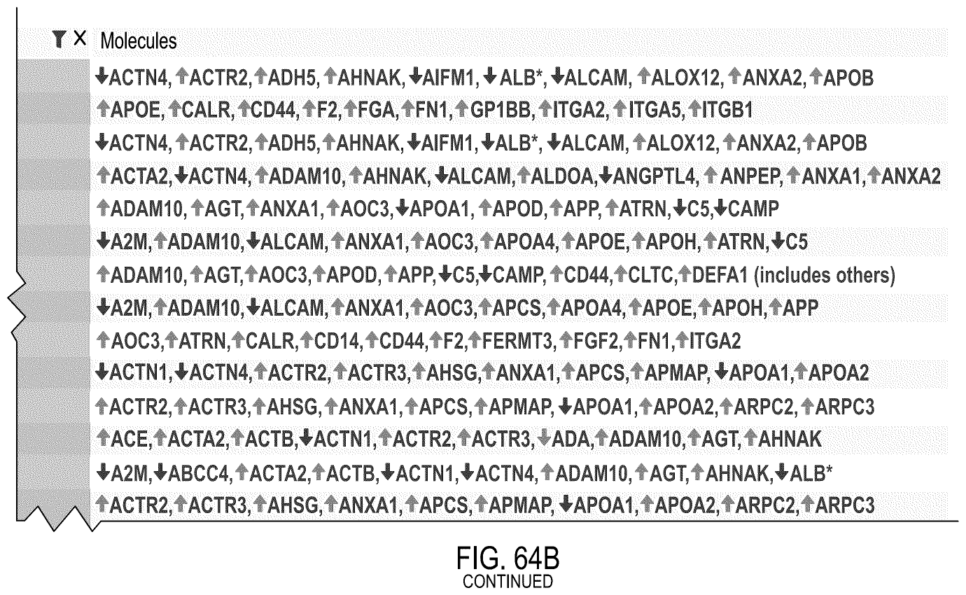

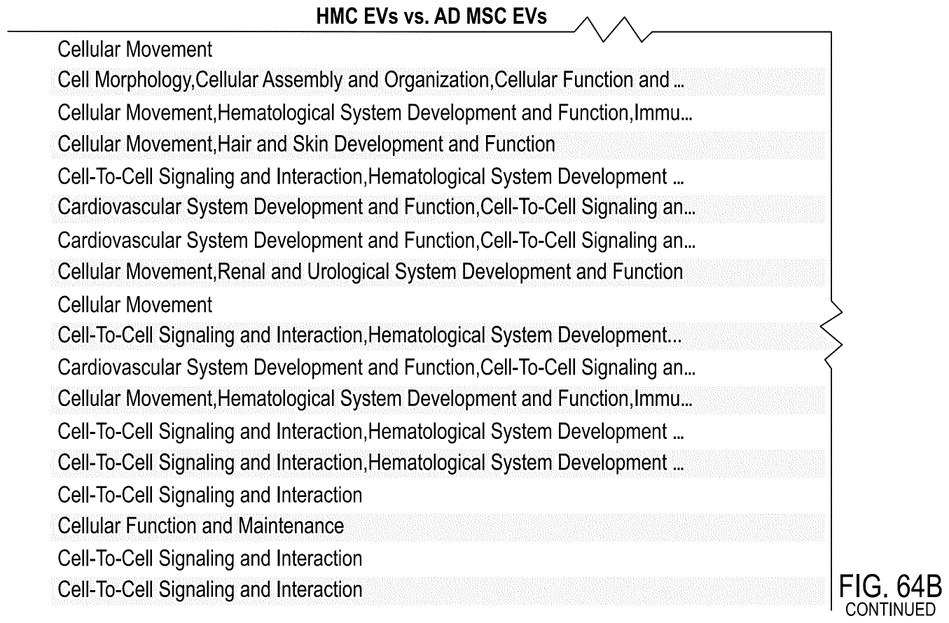

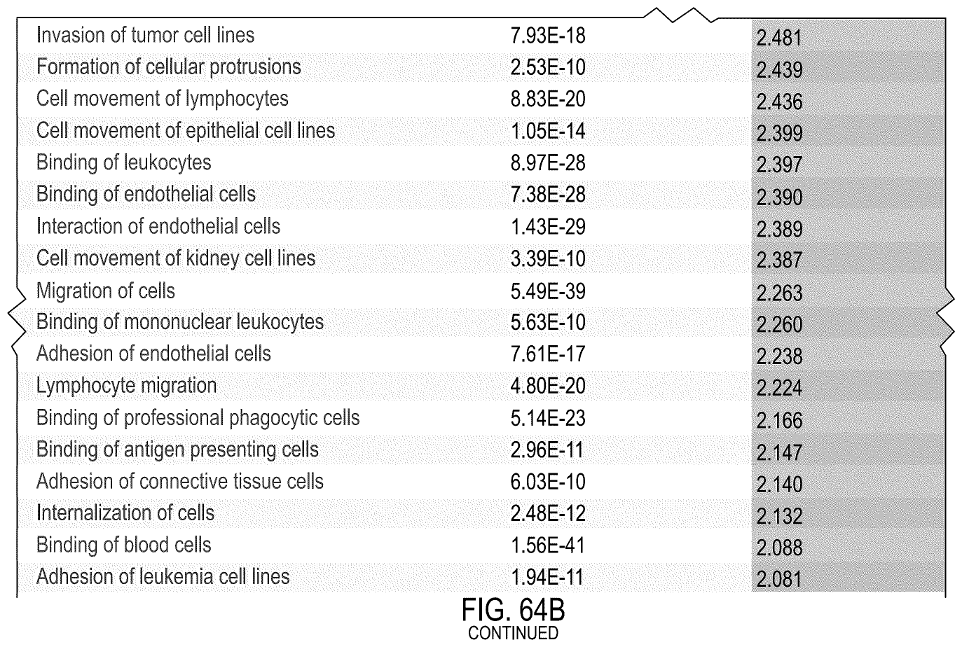

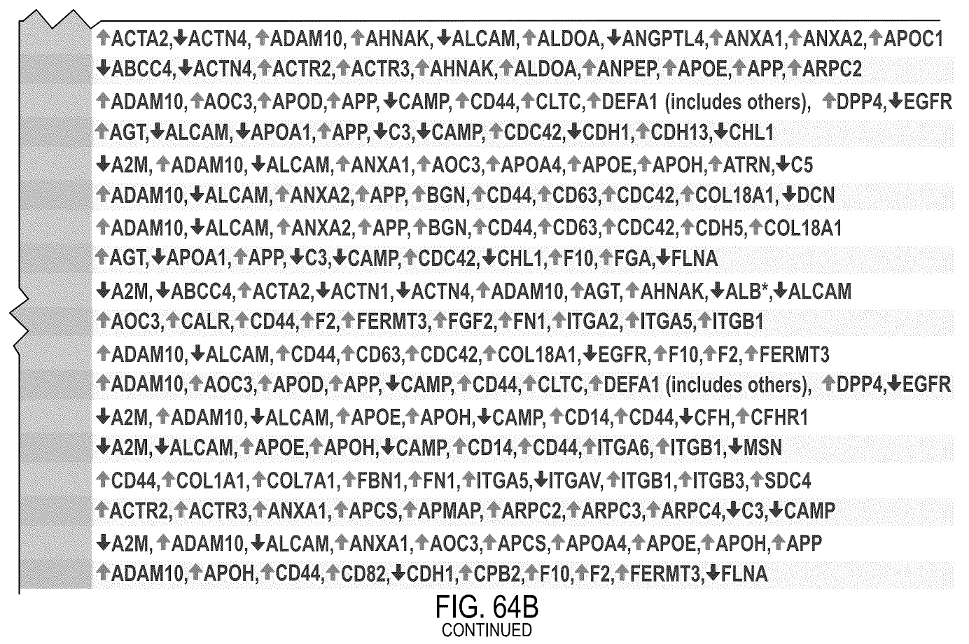

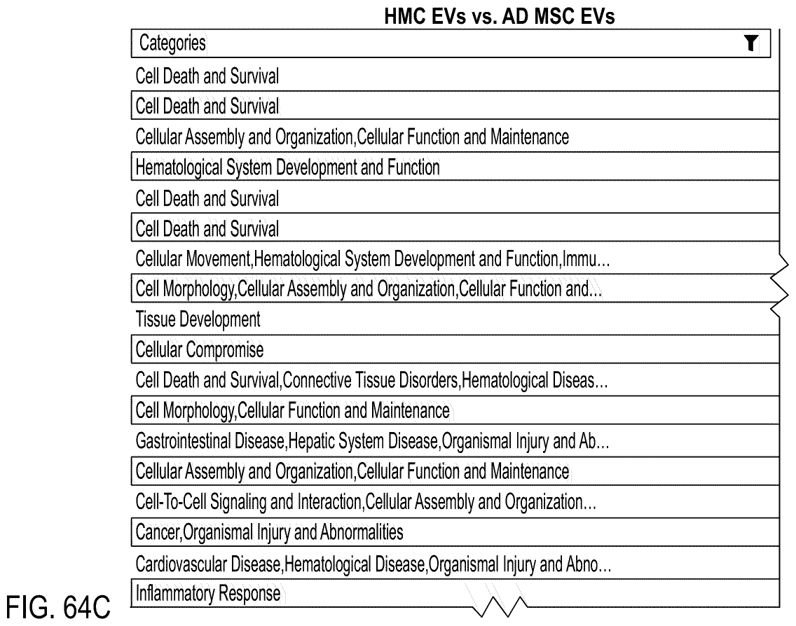

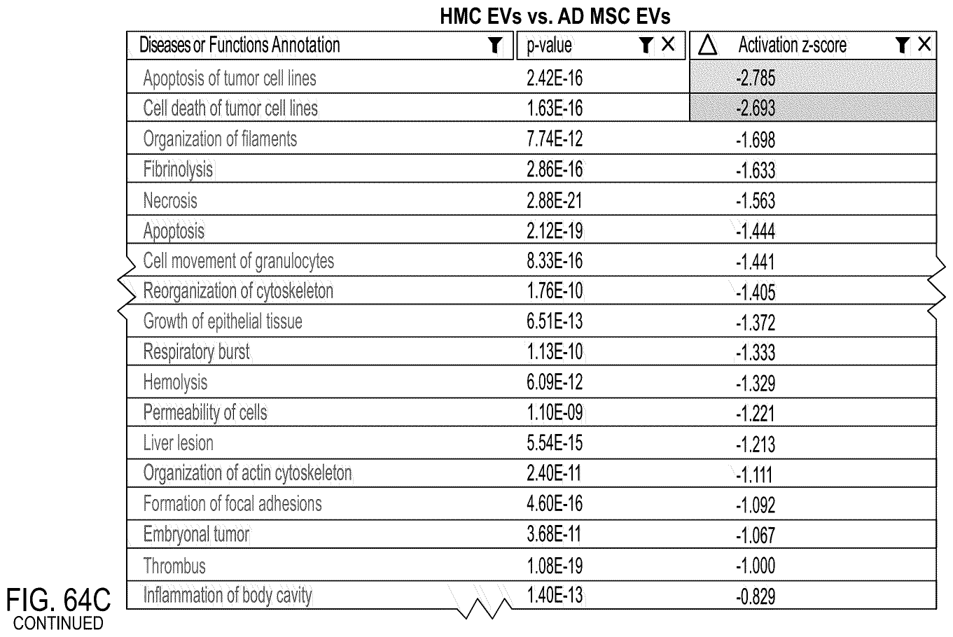

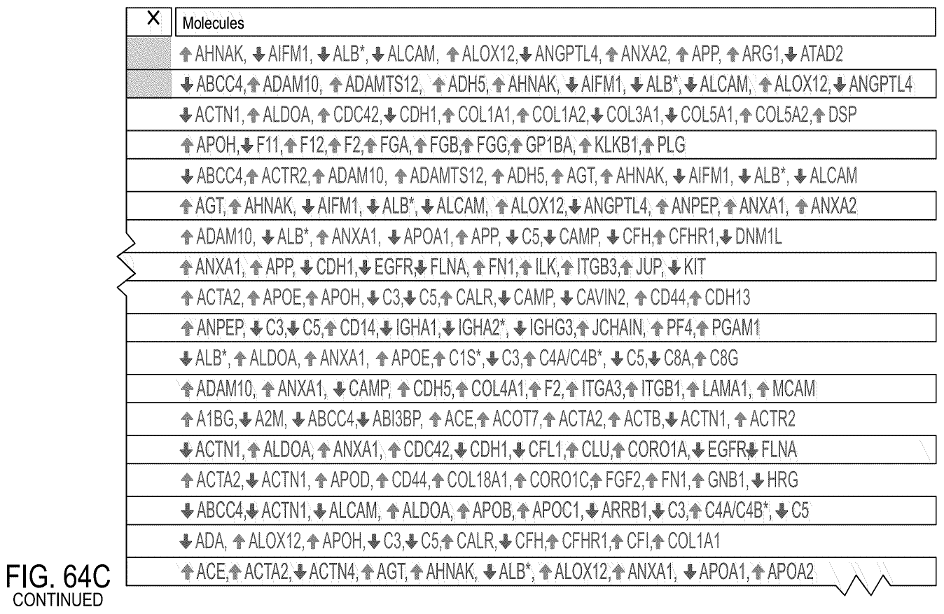

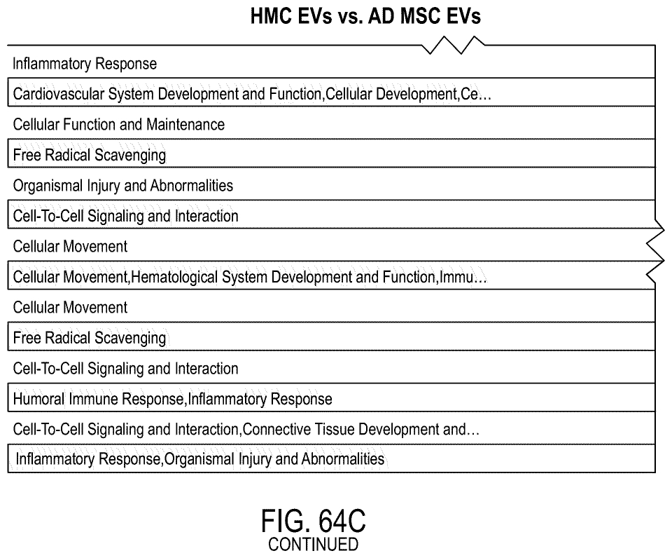

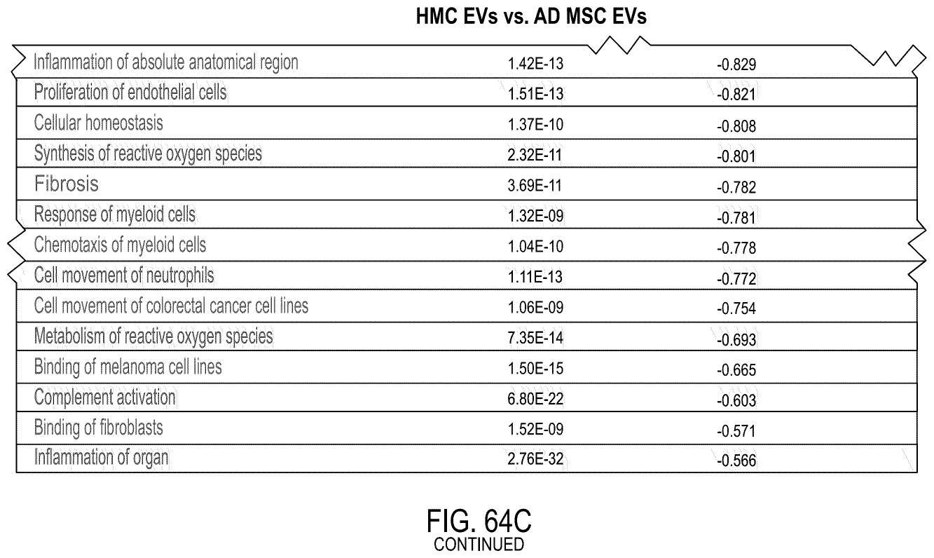

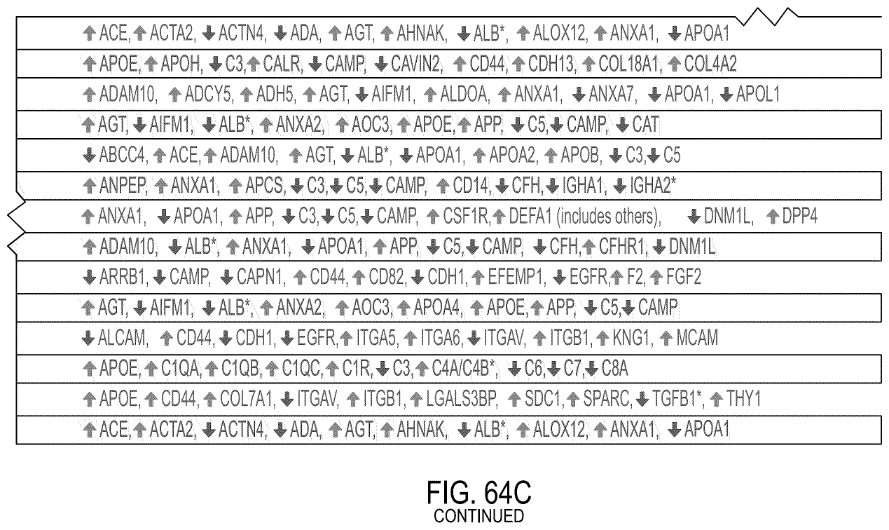

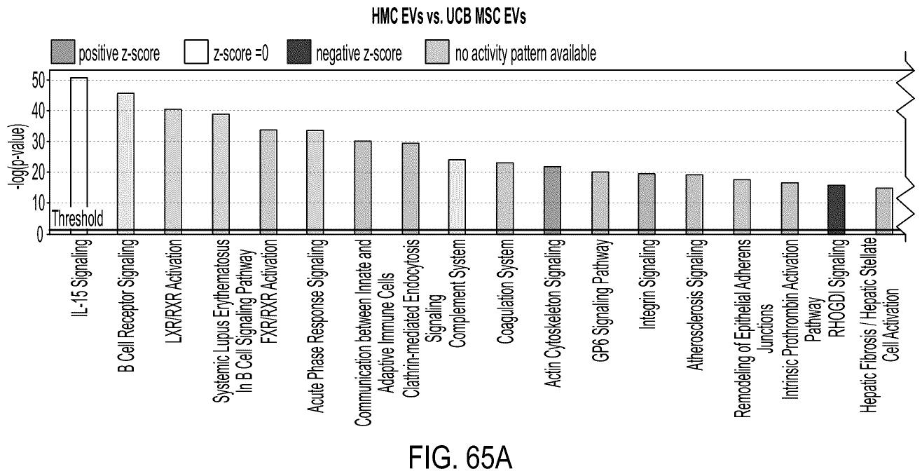

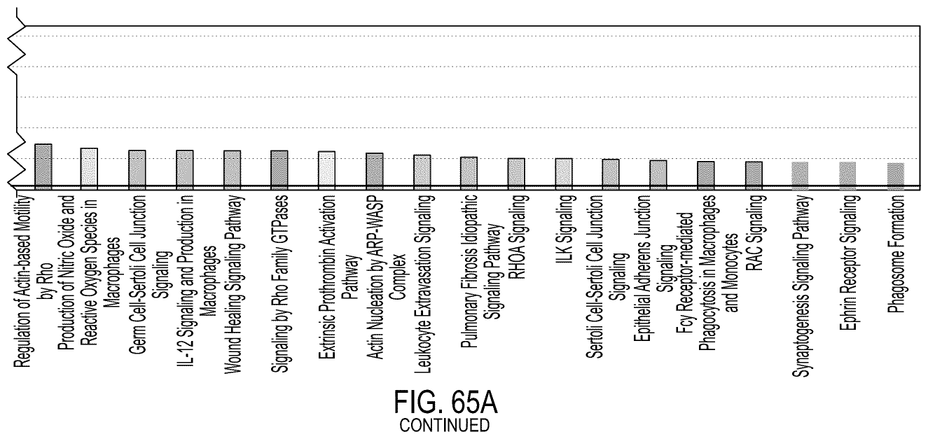

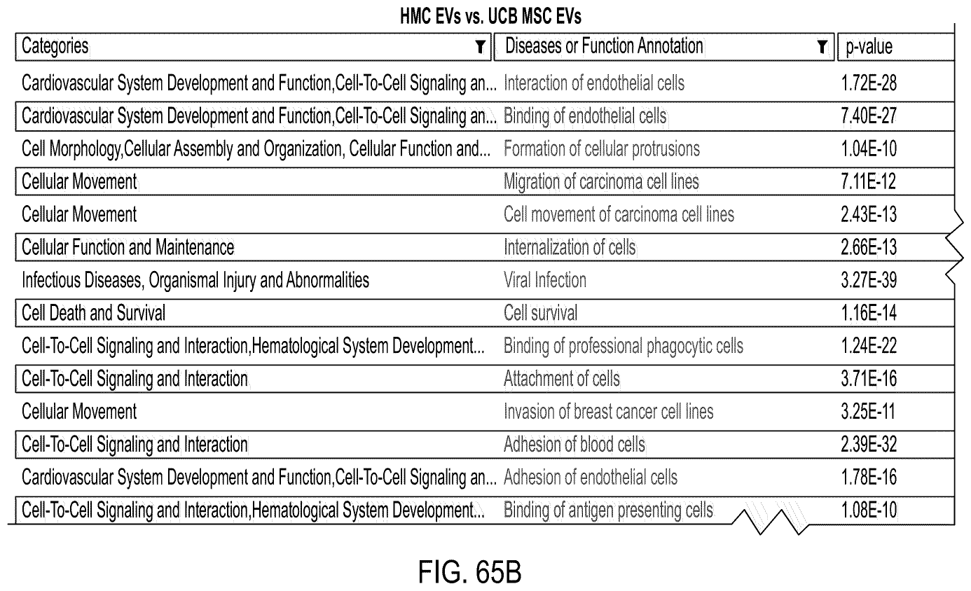

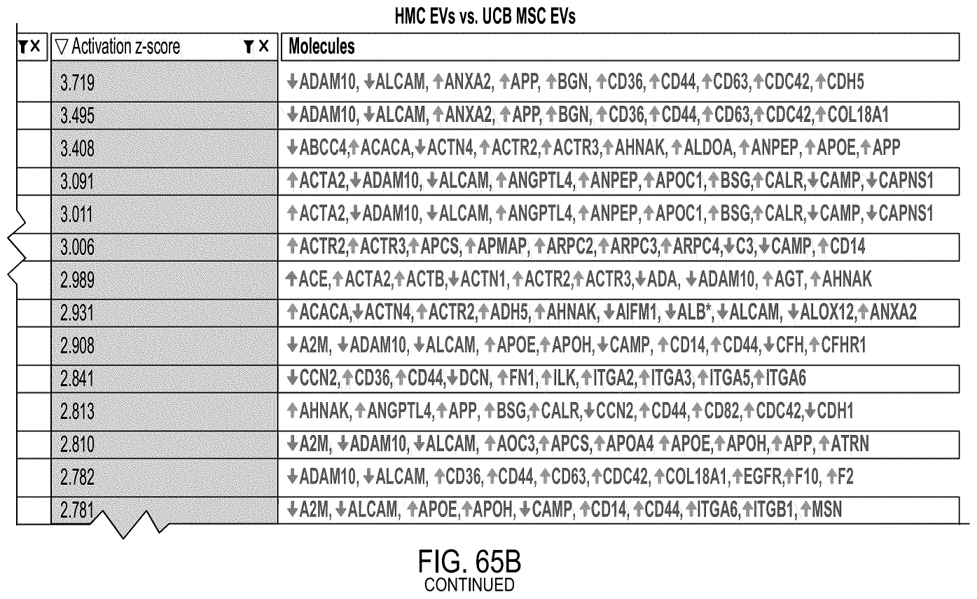

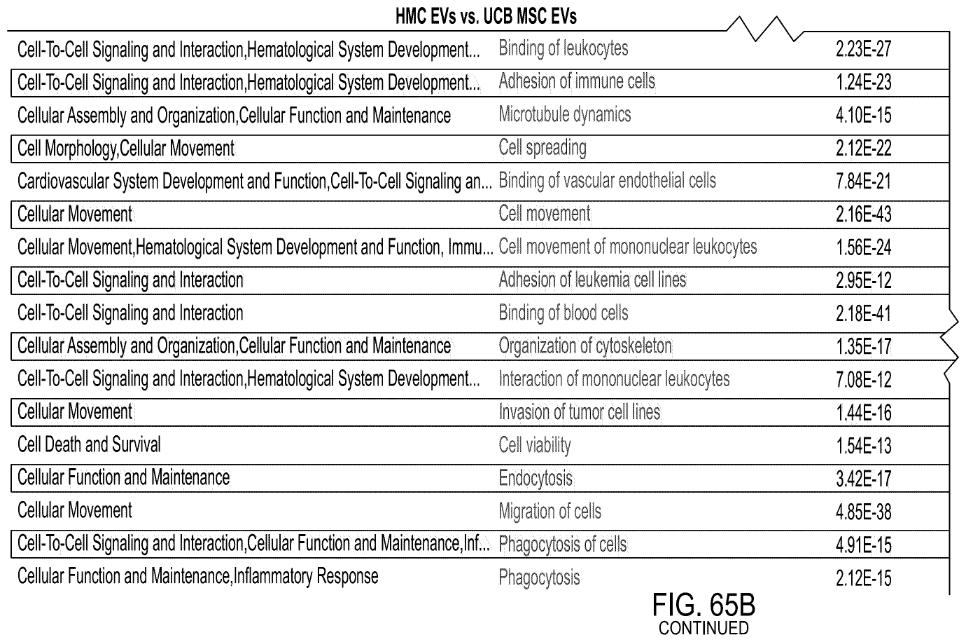

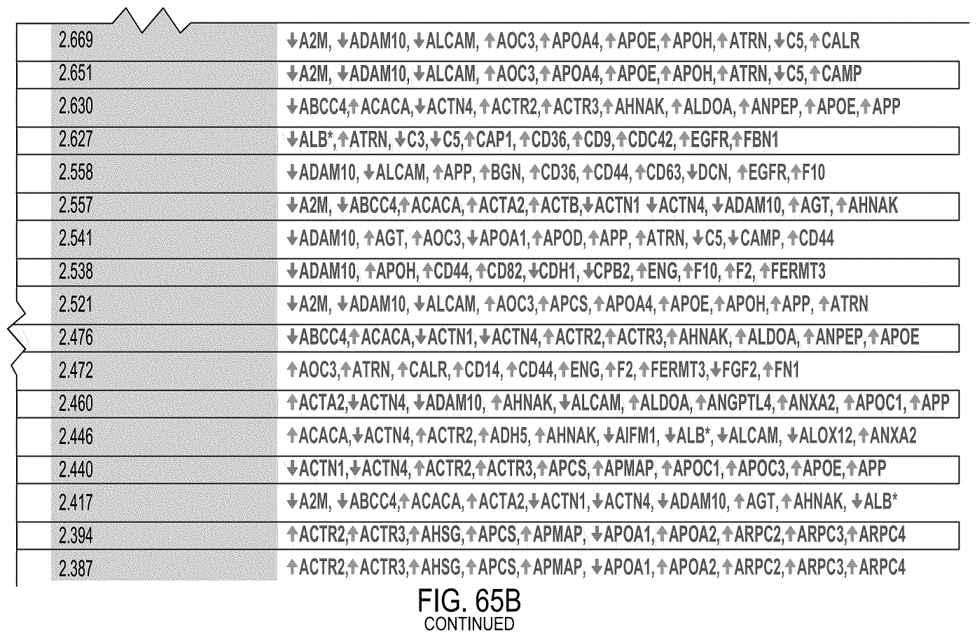

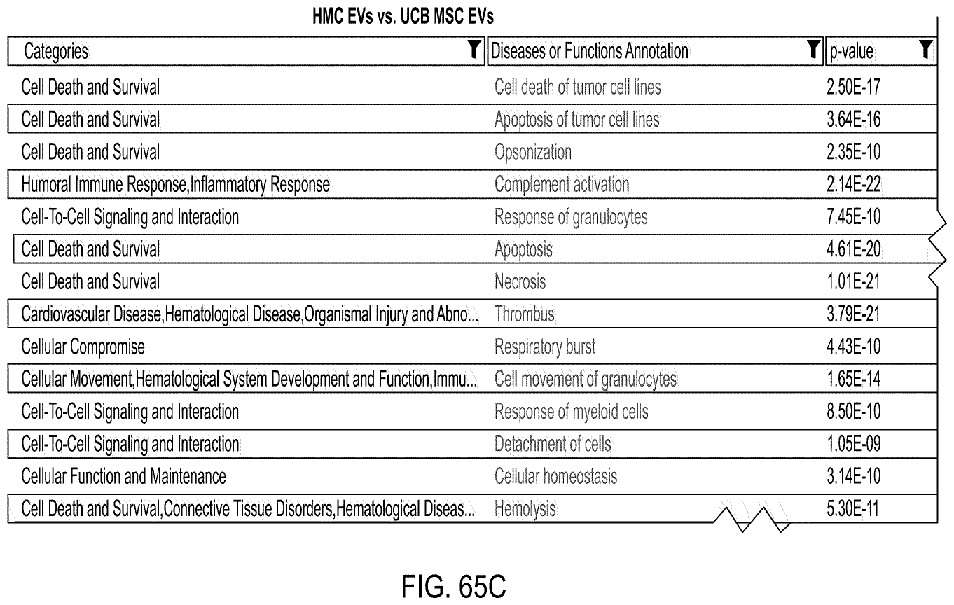

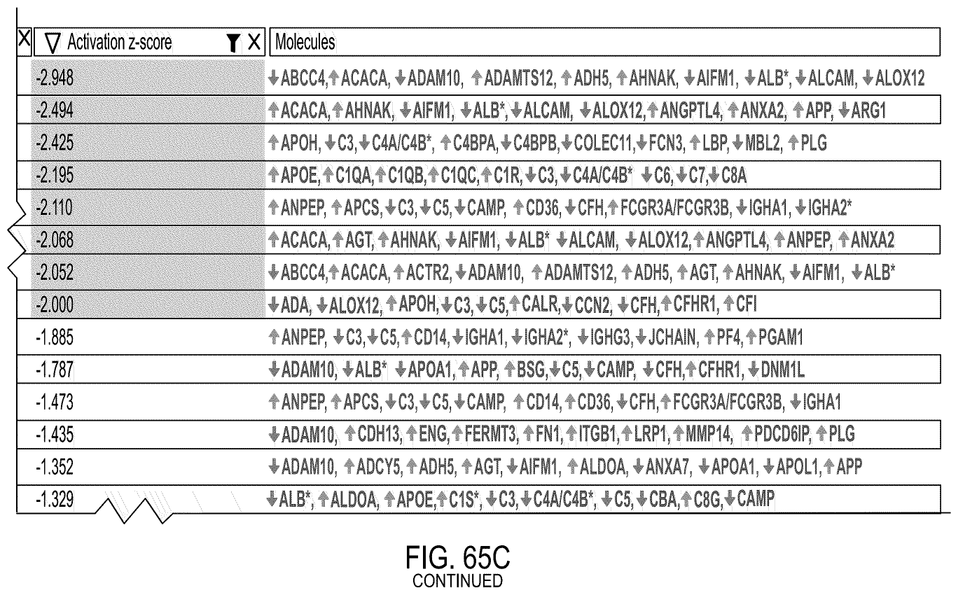

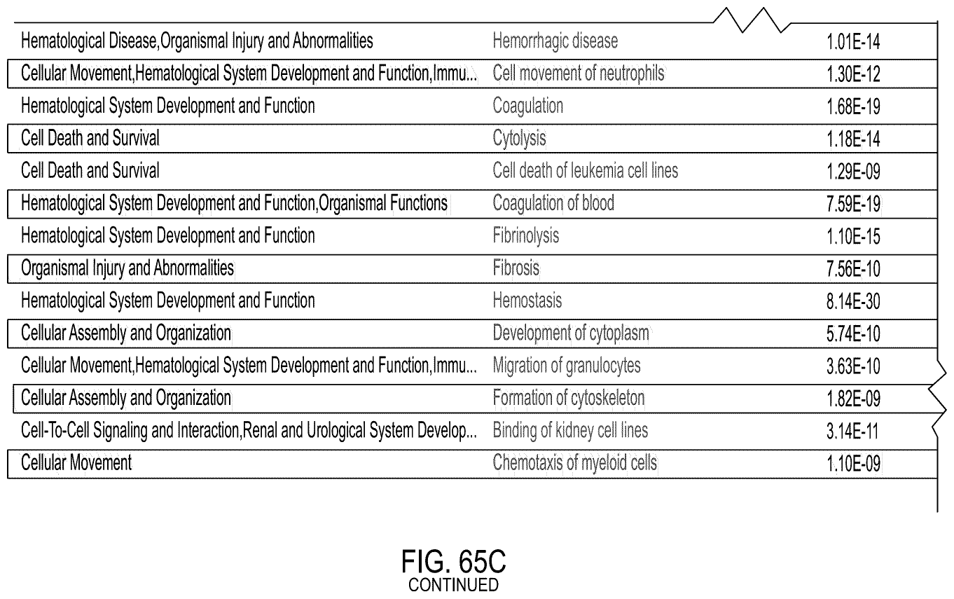

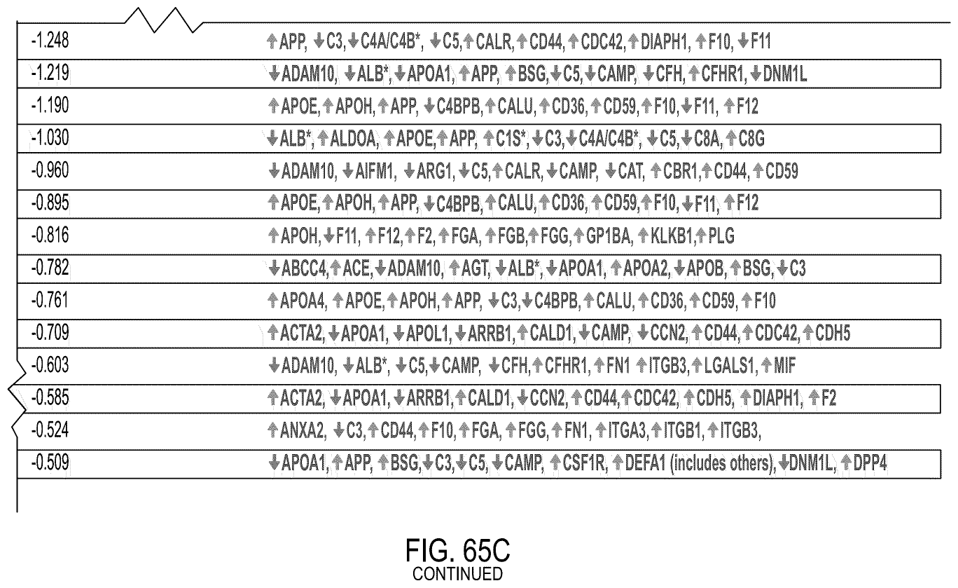

The proteomics data was subsequently analysed to determine how the overall protein expression profile may affect different signaling pathways. FIG. 63A depicts the pathway enrichment of differential expression pattern between HMC-EVs and BM-MSC-EVs. FIG. 64A depicts the pathway enrichment of differential expression pattern between HMC-EVs and AD-MSC-EVs. FIG. 65A depicts the pathway enrichment of differential expression pattern between HMC-EVs and EVs secreted from umbilical cord blood-derived MSCs (UCB-MSC-EVs). As shown in FIGS. 63A, 64A and 65A, certain pathways are up-regulated (see orange bars) in HMC-EVs as compared to EVs secreted from other tissue-derived MSCs, such as pathways involved in LXR/RXR activation, acute phase response signaling, B cell receptor signaling, and systemic lupus erythematosus in B cell signaling pathway. In addition, proteins contributing to certain pathways, for example, IL-15 signaling, claritin-mediated endocytosis signaling, and FXR/RXR activation, are also enriched (see white and gray bars). etc

Diseases or functional annotation of proteins that are differentially expressed in HMC-EVs and EVs secreted from tissue-derived MSCs are also analyzed. FIG. 63B depicts the functional annotation of proteins that are upregulated in HMC-EVs when compared to BM-MSC-EVs. FIG. 63C depicts the functional annotation of proteins that are downregulated in HMC-EVs when compared to BM-MSC-EVs. FIG. 64B depicts the functional annotation of proteins that are upregulated in HMC-EVs when compared to AD-MSC-EVs. FIG. 64C depicts the functional annotation of proteins that are downregulated in HMC-EVs when compared to AD-MSC-EVs. FIG. 65B depicts the functional annotation of proteins that are upregulated in HMC-EVs when compared to UCB-MSC-EVs. FIG. 65C depicts the functional annotation of proteins that are downregulated in HMC-EVs when compared to UCB-MSC-EVs. An activation z-score above 2 or below −2 is considered as the threshold value. The analysis suggests that proteins involved in cell viability/survival, cellular movement, cell-to-cell signalizing and interaction pathways are upregulated in HMC-EVs, whereas proteins involved in cell death or apoptosis are downregulated in HMC-EVs.

TABLE 15
Proteins significantly more abundant
in HMC-EVs compared to UCB-MSC EVs

| Name | log 2 fold difference | p-value |
|---|---|---|
| SLC2A1 | 5.55 | 1.62E−05 |
| MFGE8 | 5.07 | 2.17E−04 |
| MAMDC2 | 4.72 | 2.05E−03 |
| H3-3A | 4.31 | 2.35E−04 |
| MARCKSL1 | 4.11 | 1.78E−04 |
| KIF11 | 4.08 | 3.76E−05 |
| PRSS23 | 3.97 | 2.13E−02 |
| SLC3A2 | 3.95 | 2.18E−03 |
| CD81 | 3.85 | 2.02E−03 |
| TSPAN14 | 3.84 | 7.49E−05 |
| CD99 | 3.79 | 3.63E−03 |
| MDGA1 | 3.78 | 1.66E−03 |
| RPS18 | 3.76 | 5.81E−04 |
| CAV1 | 3.70 | 1.10E−03 |
| KRT4 | 3.69 | 3.30E−04 |
| MVP | 3.57 | 7.81E−04 |
| KPNA2 | 3.47 | 1.25E−03 |
| HLA-A | 3.47 | 1.10E−02 |
| TRIM5 | 3.46 | 6.75E−04 |
| KRAS | 3.46 | 1.36E−04 |
| ANXA5 | 3.35 | 2.75E−03 |
| GNG12 | 3.32 | 8.10E−05 |
| S100A11 | 3.31 | 2.61E−03 |
| H4-16 | 3.22 | 2.39E−05 |
| PCDH1 | 3.19 | 2.16E−03 |
| ITGAV | 3.17 | 1.55E−03 |
| H3-7 | 3.11 | 7.29E−04 |
| TNC | 3.09 | 2.30E−02 |
| VAT1 | 3.09 | 2.25E−04 |
| RAP2A | 3.06 | 2.12E−03 |
| UCHL1 | 3.01 | 3.08E−03 |
| FDPS | 3.01 | 2.08E−03 |
| H2AC20 | 3.01 | 4.80E−03 |
| RPS4X | 3.00 | 4.96E−03 |
| BASP1 | 2.99 | 2.49E−06 |
| CKM | 2.97 | 1.41E−03 |
| B2M | 2.89 | 1.16E−02 |
| TSPAN9 | 2.89 | 8.06E−04 |
| RPS3A | 2.83 | 3.59E−03 |
| RPS13 | 2.82 | 4.48E−03 |
| MMP14 | 2.78 | 2.06E−06 |
| GNAI2 | 2.77 | 2.88E−05 |
| YWHAQ | 2.77 | 4.56E−03 |
| PDIA3 | 2.75 | 7.76E−03 |
| RALA | 2.75 | 5.45E−03 |
| RPS3 | 2.74 | 1.77E−03 |
| EPB41L3 | 2.70 | 1.75E−03 |
| SLC44A1 | 2.70 | 2.09E−03 |
| ARL8A | 2.69 | 5.53E−03 |
| H1-3 | 2.69 | 1.27E−03 |
| NIBAN2 | 2.64 | 2.11E−05 |
| ITGA2 | 2.63 | 5.60E−06 |
| TUBB3 | 2.63 | 1.77E−02 |
| BBS1 | 2.62 | 5.29E−03 |
| MAPK3 | 2.61 | 4.10E−03 |
| YWHAB | 2.58 | 5.14E−03 |
| H2BC15 | 2.58 | 1.87E−05 |
| TRPM2 | 2.52 | 9.52E−03 |
| GALE | 2.49 | 2.26E−04 |
| CA2 | 2.49 | 2.81E−04 |
| H2AC21 | 2.48 | 1.22E−02 |
| TTYH3 | 2.45 | 1.23E−05 |
| PDGFRB | 2.44 | 2.84E−05 |
| CD47 | 2.41 | 5.97E−05 |
| DTD1 | 2.41 | 3.62E−03 |
| GP9 | 2.39 | 2.26E−03 |
| TAGLN2 | 2.38 | 1.02E−02 |
| GNAQ | 2.37 | 4.97E−03 |
| PPP2R1A | 2.37 | 2.16E−02 |
| ALDOC | 2.36 | 1.08E−03 |
| RPS15A | 2.35 | 8.55E−03 |
| MERTK | 2.35 | 4.34E−03 |
| MDH1 | 2.34 | 1.63E−05 |
| TPT1 | 2.27 | 1.35E−03 |
| EIF5A | 2.21 | 2.70E−02 |
| LYN | 2.20 | 2.74E−02 |
| VCAN | 2.18 | 7.73E−09 |
| CYFIP1 | 2.18 | 5.89E−03 |
| APBB2 | 2.18 | 1.93E−02 |
| SDCBP | 2.16 | 1.56E−06 |
| LAP3 | 2.16 | 2.08E−02 |
| KRT13 | 2.15 | 3.25E−02 |
| LRRC59 | 2.13 | 5.62E−03 |
| RPL13 | 2.13 | 4.00E−04 |
| CD36 | 2.12 | 2.49E−05 |
| SRSF8 | 2.08 | 2.45E−05 |
| TSPAN33 | 2.03 | 4.53E−03 |
| TPTE2 | 2.03 | 2.17E−02 |
| HLA-A | 2.02 | 1.02E−02 |
| EPHB2 | 1.93 | 9.56E−03 |
| FAH | 1.93 | 4.25E−03 |
| FUCA1 | 1.90 | 3.24E−04 |
| MARCKS | 1.89 | 2.79E−05 |
| GP1BB | 1.88 | 2.91E−04 |
| CD276 | 1.88 | 1.48E−03 |
| ACLY | 1.86 | 2.15E−02 |
| YWHAE | 1.86 | 1.50E−02 |
| PLAA | 1.85 | 2.05E−02 |
| UBE2L3 | 1.85 | 2.75E−02 |
| WARS1 | 1.83 | 9.19E−04 |
| AOC3 | 1.83 | 3.71E−05 |
| BGN | 1.82 | 1.55E−07 |
| AGRN | 1.82 | 8.24E−06 |
| SLC44A2 | 1.78 | 3.13E−02 |
| RPL11 | 1.77 | 1.77E−02 |
| FARP1 | 1.73 | 5.55E−03 |
| ITGA3 | 1.72 | 6.48E−07 |
| ANXA2 | 1.71 | 3.90E−05 |
| STX11 | 1.71 | 4.14E−05 |
| TBC1D2 | 1.71 | 1.96E−02 |
| PGAP1 | 1.71 | 5.86E−06 |
| RPL14 | 1.68 | 1.27E−02 |
| RPL10A | 1.66 | 9.01E−06 |
| CD63 | 1.66 | 5.06E−05 |
| CPN2 | 1.63 | 4.56E−06 |
| RPS25 | 1.62 | 8.88E−03 |
| BLVRA | 1.62 | 3.32E−03 |
| PPP2CB | 1.62 | 2.58E−04 |
| LGALS1 | 1.59 | 1.48E−05 |
| S100A6 | 1.51 | 2.82E−07 |
| MEIOB | 1.51 | 4.95E−06 |
| NME2 | 1.51 | 2.23E−03 |
| OSMR | 1.50 | 2.78E−02 |
| SEPTIN5 | 1.49 | 6.58E−03 |
| MYL12B | 1.49 | 1.38E−03 |
| FN3KRP | 1.49 | 9.63E−03 |
| CDH5 | 1.48 | 4.17E−05 |
| ITGA2B | 1.48 | 1.11E−05 |
| HLA-B | 1.47 | 1.33E−04 |
| BMP1 | 1.46 | 4.26E−02 |
| CLIC4 | 1.45 | 1.31E−03 |
| BST1 | 1.45 | 1.05E−03 |
| ITGB1 | 1.45 | 6.19E−11 |
| STRADB | 1.44 | 2.93E−04 |
| MOB1B | 1.43 | 5.59E−03 |
| SDC1 | 1.43 | 4.81E−03 |
| B4GALT1 | 1.42 | 3.27E−03 |
| ITGA6 | 1.38 | 2.40E−09 |
| RPL4 | 1.36 | 3.19E−02 |
| ITGA4 | 1.33 | 4.40E−04 |
| COL4A2 | 1.33 | 3.97E−07 |
| PDCD6IP | 1.32 | 1.34E−07 |
| MSN | 1.32 | 1.34E−07 |
| PF4 | 1.32 | 2.10E−03 |
| STXBP2 | 1.31 | 1.64E−02 |
| ARF6 | 1.30 | 3.43E−02 |
| EDIL3 | 1.29 | 1.12E−02 |
| COTL1 | 1.29 | 1.54E−02 |
| ITGA5 | 1.28 | 1.34E−05 |
| QSOX1 | 1.28 | 3.43E−04 |
| RALB | 1.28 | 1.71E−04 |
| NAGLU | 1.28 | 6.87E−04 |
| GNB1 | 1.28 | 3.11E−03 |
| PDE4DIP | 1.27 | 2.47E−04 |
| CBR1 | 1.27 | 3.98E−03 |
| ROBO4 | 1.26 | 8.58E−04 |
| FBLN1 | 1.25 | 1.33E−02 |
| STOM | 1.25 | 2.40E−05 |
| SRPX | 1.25 | 4.98E−04 |
| GSTM2 | 1.24 | 1.05E−04 |
| ZNF607 | 1.23 | 7.88E−05 |
| KIT | 1.21 | 8.09E−05 |
| LAMP1 | 1.21 | 7.48E−03 |
| SEPTIN2 | 1.21 | 3.37E−04 |
| CDC42 | 1.20 | 2.49E−03 |
| ANXA6 | 1.19 | 3.38E−05 |
| TANK | 1.17 | 1.89E−05 |
| UBA52 | 1.17 | 2.31E−04 |
| COL18A1 | 1.17 | 1.41E−02 |
| PAFAH1B1 | 1.15 | 3.38E−02 |
| NUTF2 | 1.15 | 5.64E−04 |
| TPI1 | 1.14 | 3.11E−07 |
| LRP1 | 1.14 | 1.21E−04 |
| SERPINA10 | 1.14 | 8.66E−03 |
| MYO1F | 1.13 | 2.84E−03 |
| VNN1 | 1.12 | 1.99E−04 |
| RPSA | 1.12 | 1.82E−04 |
| ARPC5 | 1.12 | 2.29E−02 |
| CTBS | 1.11 | 1.26E−07 |
| MON2 | 1.11 | 1.38E−05 |
| LUM | 1.10 | 1.24E−03 |
| RPS12 | 1.08 | 5.10E−03 |
| PGLYRP2 | 1.08 | 5.38E−05 |
| APOC4 | 1.08 | 9.84E−04 |
| BANF1 | 1.08 | 1.55E−02 |
| PRG4 | 1.07 | 3.13E−02 |
| SERPINE2 | 1.07 | 1.26E−02 |
| AHSG | 1.07 | 2.87E−07 |
| DYNLL1 | 1.06 | 3.53E−06 |
| RAC1 | 1.06 | 3.65E−04 |
| PRKAR1A | 1.06 | 2.75E−03 |
| SH3BGRL3 | 1.05 | 1.10E−03 |
| CD9 | 1.04 | 7.31E−08 |
| CLPP | 1.04 | 9.54E−03 |
| DEFA3 | 1.03 | 2.28E−02 |
| CCT4 | 1.03 | 1.79E−04 |
| HSPA4L | 1.03 | 5.99E−04 |
| EFEMP1 | 1.02 | 2.90E−02 |
| GLIPR2 | 1.02 | 1.07E−03 |
| ITGB3 | 1.02 | 1.86E−05 |
| FUCA2 | 1.01 | 1.66E−02 |
| PROCR | 1.01 | 9.71E−05 |
| CFHR1 | 1.00 | 1.67E−04 |
| YWHAZ | 1.00 | 3.72E−06 |

TABLE 16
Proteins significantly more abundant
in UCB-MSC EVs compared to HMC-EVs

| Name | log 2 fold difference | p-value |
|---|---|---|
| TMEM198 | −5.16 | 3.92E−10 |
| CAT | −5.16 | 1.45E−06 |
| SPON2 | −4.11 | 5.60E−05 |
| DOK4 | −4.09 | 3.21E−05 |
| LRAT | −3.88 | 3.31E−05 |
| ADIPOQ | −3.85 | 3.79E−04 |
| PTX3 | −3.69 | 7.69E−06 |
| CHST9 | −3.52 | 5.30E−07 |
| CEP290 | −3.46 | 3.05E−03 |
| FAM151B | −3.41 | 1.76E−02 |
| IGHV1-45 | −3.36 | 1.71E−02 |
| MSH6 | −3.22 | 7.41E−03 |
| SNTG1 | −3.11 | 4.29E−06 |
| AKAP9 | −2.92 | 3.94E−06 |
| MUC16 | −2.91 | 2.71E−03 |
| ALB | −2.87 | 5.77E−04 |
| LRRTM2 | −2.79 | 8.46E−05 |
| SURF1 | −2.77 | 1.45E−02 |
| CDSN | −2.76 | 1.11E−02 |
| PSMA6 | −2.73 | 7.91E−05 |
| F11 | −2.68 | 4.35E−08 |
| ALOX5 | −2.63 | 3.36E−06 |
| SEMA7A | −2.52 | 1.92E−02 |
| TAS2R33 | −2.50 | 2.27E−03 |
| IGHV3-38-3 | −2.48 | 1.14E−03 |
| TYMP | −2.47 | 7.15E−06 |
| MMRN2 | −2.47 | 1.11E−02 |
| PAK6 | −2.46 | 4.81E−03 |
| LDLR | −2.46 | 1.24E−02 |
| KRT17 | −2.45 | 3.78E−02 |
| CCIN | −2.45 | 1.39E−03 |
| RGS14 | −2.39 | 5.06E−03 |
| TRIM4 | −2.38 | 7.42E−03 |
| CFHR5 | −2.38 | 1.71E−02 |
| AP3B2 | −2.34 | 1.05E−02 |
| TIMP3 | −2.34 | 3.57E−04 |
| L1CAM | −2.31 | 3.56E−06 |
| IGHV3OR16-13 | −2.27 | 3.03E−02 |
| ABI3 | −2.24 | 1.71E−03 |
| BLMH | −2.20 | 3.37E−03 |
| S100A9 | −2.19 | 3.76E−06 |
| LAMB4 | −2.16 | 1.42E−02 |
| LTF | −2.15 | 2.62E−02 |
| ERC1 | −2.14 | 1.10E−02 |
| APLP2 | −2.12 | 6.31E−03 |
| ZSWIM9 | −2.11 | 7.12E−03 |
| OLFML3 | −2.10 | 1.82E−02 |
| CTHRC1 | −2.10 | 1.79E−05 |
| CD109 | −2.07 | 1.92E−02 |
| IGLV6-57 | −2.04 | 4.16E−04 |
| REG1A | −2.02 | 1.27E−02 |
| CCBE1 | −2.02 | 1.36E−02 |
| OAF | −2.01 | 2.28E−05 |
| NEO1 | −1.97 | 2.41E−02 |
| NBEAL2 | −1.92 | 1.99E−02 |
| PIWIL2 | −1.84 | 3.95E−05 |
| SBSN | −1.82 | 4.12E−02 |
| CAPN5 | −1.80 | 1.04E−08 |
| TRIM7 | −1.76 | 1.08E−06 |
| ZNF804B | −1.73 | 1.35E−03 |
| LYVE1 | −1.72 | 4.57E−04 |
| ACTR1A | −1.70 | 1.16E−02 |
| IGHG2 | −1.67 | 9.34E−10 |
| DSC1 | −1.66 | 2.60E−04 |
| PDZK1P1 | −1.63 | 8.47E−04 |
| FHL1 | −1.61 | 1.39E−02 |
| PSMA7 | −1.58 | 1.94E−07 |
| DBH | −1.55 | 1.42E−03 |
| IGHV3-74 | −1.53 | 2.05E−05 |
| PRXL2B | −1.53 | 2.10E−07 |
| C18orf63 | −1.51 | 5.55E−06 |
| IGHG1 | −1.48 | 2.23E−09 |
| PSMA4 | −1.45 | 3.77E−03 |
| UBTD1 | −1.45 | 2.11E−06 |
| PIEZO1 | −1.44 | 1.14E−05 |
| MYCBP2 | −1.43 | 1.76E−02 |
| NYAP2 | −1.43 | 2.19E−06 |
| CCDC110 | −1.42 | 1.18E−05 |
| ZNF800 | −1.41 | 1.95E−07 |
| VEGFA | −1.41 | 3.31E−02 |
| FBRSL1 | −1.41 | 1.61E−04 |
| GTF2IRD2 | −1.39 | 1.99E−06 |
| PPM1F | −1.39 | 4.41E−02 |
| HGFAC | −1.37 | 5.90E−03 |
| IGLV3-1 | −1.36 | 8.16E−04 |
| CD99L2 | −1.36 | 6.57E−06 |
| L1TD1 | −1.35 | 4.40E−11 |
| KRT16 | −1.34 | 2.76E−03 |
| XPNPEP2 | −1.34 | 2.62E−05 |
| IGHA2 | −1.32 | 7.71E−04 |
| ADA | −1.30 | 2.88E−07 |
| ALB | −1.30 | 1.22E−02 |
| IGLV2-18 | −1.29 | 2.05E−02 |
| IGHV4-4 | −1.28 | 2.45E−09 |
| COLEC11 | −1.27 | 1.39E−02 |
| PKP1 | −1.24 | 1.57E−03 |
| MYH3 | −1.23 | 4.39E−02 |
| TGFB1 | −1.23 | 2.74E−06 |
| IGHV1-69 | −1.23 | 1.28E−04 |
| IGLV3-21 | −1.22 | 2.11E−08 |
| DDX55 | −1.20 | 9.05E−10 |
| IGHA1 | −1.20 | 5.28E−11 |
| ANO7 | −1.20 | 1.08E−07 |
| MPP1 | −1.19 | 1.11E−03 |
| GPR179 | −1.19 | 4.94E−06 |
| WDR46 | −1.19 | 1.08E−04 |
| SYMPK | −1.18 | 2.74E−05 |
| TNFAIP6 | −1.13 | 1.92E−06 |
| RACK1 | −1.13 | 2.93E−04 |
| LOXL2 | −1.12 | 3.21E−02 |
| A2M | −1.12 | 3.96E−07 |
| S100A8 | −1.11 | 4.92E−06 |
| IGKV3D-20 | −1.11 | 6.86E−06 |
| ITIH1 | −1.10 | 1.87E−09 |
| GDI1 | −1.09 | 9.12E−06 |
| IGHV5-10-1 | −1.06 | 4.68E−05 |
| CYLC2 | −1.05 | 2.35E−05 |
| IGHD | −1.04 | 1.81E−04 |
| VTI1B | −1.04 | 8.83E−04 |
| VCP | −1.03 | 9.97E−07 |
| USP4 | −1.03 | 1.72E−04 |
| ATAD2 | −1.03 | 1.41E−05 |
| TF | −1.03 | 4.24E−08 |
| F13B | −1.03 | 7.08E−05 |
| ITIH3 | −1.02 | 1.71E−06 |
| IGLV3-25 | −1.02 | 1.38E−07 |
| CCT6A | −1.01 | 1.46E−02 |
| CFH | −1.00 | 2.81E−06 |
| IGLV3-27 | −1.00 | 2.68E−06 |

TABLE 17
Proteins significantly more abundant
in HMC-EVs compared to BM-MSC EVs

| Name | log 2 fold difference | p-value |
|---|---|---|
| GDF10 | 9.24 | 9.5E−08 |
| L1TD1 | 7.45 | 0.004719 |
| CD82 | 7.27 | 6.06E−07 |
| ZNF607 | 7.18 | 6.84E−07 |
| KRT78 | 6.83 | 2.14E−07 |
| H2AC20 | 6.53 | 1.75E−06 |
| IGKV1-17 | 6.46 | 5.34E−05 |
| GATA5 | 6.34 | 2.64E−06 |
| H3-3A | 6.10 | 5.68E−05 |
| GOLGB1 | 5.73 | 4.16E−06 |
| CCT4 | 5.39 | 4.42E−06 |
| DYNLL1 | 5.38 | 0.000324 |
| ARHGDIA | 5.36 | 0.000256 |
| B4GALT1 | 5.26 | 2.07E−05 |
| LTBP3 | 5.17 | 0.008415 |
| CORO1A | 5.15 | 6.22E−10 |
| ADGRG6 | 5.14 | 1.64E−07 |
| PRDM5 | 5.11 | 3.57E−06 |
| STAC2 | 5.10 | 4.11E−05 |
| IGLV2-14 | 5.10 | 0.000465 |
| ROBO4 | 5.04 | 7.26E−09 |
| MBTD1 | 5.03 | 8.28E−06 |
| CHMP4B | 4.98 | 0.002437 |
| IGHV5-10-1 | 4.93 | 0.000306 |
| FAM76A | 4.93 | 2.98E−06 |
| C4B_2 | 4.86 | 0.000418 |
| OBSCN | 4.81 | 5.91E−05 |
| N4BP1 | 4.79 | 0.002836 |
| VCP | 4.76 | 0.000141 |
| MYEF2 | 4.65 | 0.014568 |
| EXOC1 | 4.62 | 1.8E−07 |
| IGHV4-4 | 4.61 | 0.000758 |
| SLC2A1 | 4.38 | 4.24E−06 |
| SPARC | 4.33 | 6.11E−08 |
| FBLN1 | 4.32 | 2.04E−07 |
| NBPF4 | 4.30 | 0.014757 |
| BASP1 | 4.29 | 0.002139 |
| MYO1F | 4.22 | 4.22E−06 |
| PIK3CA | 4.14 | 0.000126 |
| STRADB | 4.11 | 0.00015 |
| MERTK | 4.10 | 7.49E−05 |
| DENND1B | 4.08 | 0.000505 |
| COL4A1 | 4.08 | 8.57E−05 |
| SLTM | 4.05 | 1.03E−05 |
| LGALS1 | 4.03 | 0.010051 |
| CFHR1 | 4.03 | 3.92E−05 |
| TSPAN14 | 3.92 | 3.45E−05 |
| MARCKSL1 | 3.91 | 1.26E−05 |
| CAV1 | 3.83 | 7.42E−05 |
| ZNF879 | 3.81 | 5.73E−05 |
| MIF | 3.78 | 0.00036 |
| MVP | 3.78 | 2.82E−06 |
| STXBP2 | 3.77 | 2.93E−05 |
| TAGLN2 | 3.77 | 2.81E−11 |
| MOB1B | 3.77 | 0.000694 |
| TSKU | 3.76 | 0.001976 |
| PMVK | 3.72 | 0.000187 |
| TNC | 3.71 | 0.000233 |
| GPX3 | 3.68 | 2E−06 |
| GOT1L1 | 3.68 | 6.05E−06 |
| EDIL3 | 3.68 | 1.24E−06 |
| SNX14 | 3.63 | 0.00065 |
| MYL12B | 3.63 | 0.000851 |
| KRT4 | 3.61 | 0.000113 |
| COL5A2 | 3.59 | 0.002899 |
| PRAMEF10 | 3.57 | 2.37E−05 |
| ITGA2 | 3.51 | 1E−05 |
| CDH5 | 3.48 | 0.000121 |
| APBB2 | 3.48 | 0.005607 |
| CCN2 | 3.47 | 0.000961 |
| ALOX12 | 3.45 | 0.001648 |
| SLC3A2 | 3.45 | 0.000745 |
| IGLV1-40 | 3.44 | 0.000266 |
| YWHAB | 3.44 | 0.002303 |
| H3-7 | 3.42 | 0.004263 |
| TIMP1 | 3.42 | 0.008573 |
| GNB2 | 3.41 | 0.009415 |
| VAT1 | 3.40 | 4.14E−05 |
| PLCH1 | 3.39 | 1.03E−07 |
| IGKV1D-16 | 3.39 | 0.000314 |
| SMG1 | 3.38 | 9.29E−08 |
| CALR | 3.38 | 0.005623 |
| RPS18 | 3.37 | 0.000232 |
| CYP11B1 | 3.37 | 0.00017 |
| RPSA | 3.35 | 0.008587 |
| IGHV3-64 | 3.33 | 0.001246 |
| CDH13 | 3.31 | 8.2E−09 |
| PDIA3 | 3.30 | 0.000169 |
| MMP14 | 3.26 | 0.026749 |
| PCDH1 | 3.25 | 9.86E−05 |
| MFGE8 | 3.23 | 0.00748 |
| IGHV1-18 | 3.22 | 3.63E−06 |
| IGHG4 | 3.21 | 0.000498 |
| TSPAN9 | 3.21 | 0.000157 |
| ALDOC | 3.21 | 5.46E−07 |
| BIN2 | 3.20 | 0.035938 |
| STN1 | 3.19 | 0.00191 |
| GNAQ | 3.18 | 0.002647 |
| GANAB | 3.17 | 6.5E−05 |
| ADA | 3.14 | 0.030644 |
| PF4 | 3.13 | 7.99E−08 |
| ARPC5 | 3.09 | 0.000116 |
| HLA-A | 3.09 | 0.006161 |
| APRT | 3.07 | 0.000978 |
| PAFAH1B1 | 3.07 | 1.37E−05 |
| PGAP1 | 3.06 | 8.7E−05 |
| PRG4 | 3.06 | 1.28E−05 |
| CAP1 | 3.02 | 1.92E−08 |
| COL18A1 | 3.02 | 0.015049 |
| ATP6V1E1 | 3.02 | 0.004046 |
| IGLV2-18 | 3.01 | 0.000485 |
| KPNA2 | 3.01 | 0.001586 |
| ANXA6 | 3.00 | 7E−06 |
| TRIM5 | 2.97 | 0.006958 |
| CD99 | 2.96 | 0.001694 |
| HSPB1 | 2.92 | 0.000978 |
| PXDN | 2.92 | 1.47E−06 |
| H4-16 | 2.92 | 8.26E−05 |
| PON3 | 2.91 | 2.32E−05 |
| BLVRA | 2.90 | 0.016521 |
| CLIC4 | 2.88 | 0.001057 |
| RPL18 | 2.87 | 4.68E−05 |
| YWHAE | 2.87 | 0.001475 |
| EEF1D | 2.87 | 0.001449 |
| UCHL1 | 2.85 | 0.00102 |
| SDCBP | 2.85 | 6.09E−07 |
| KIF3B | 2.84 | 0.000319 |
| APOC4 | 2.83 | 2.48E−05 |
| GPR108 | 2.83 | 0.000693 |
| MDGA1 | 2.79 | 0.013423 |
| SFRP1 | 2.79 | 0.035069 |
| LCP2 | 2.79 | 4.89E−05 |
| ANXA5 | 2.78 | 5.76E−05 |
| FGD6 | 2.77 | 0.001047 |
| DSP | 2.76 | 2.7E−05 |
| TTYH3 | 2.76 | 0.005149 |
| MMP2 | 2.75 | 0.001 |
| AEBP1 | 2.75 | 0.00897 |
| RPS3A | 2.74 | 2.67E−06 |
| RPLP2 | 2.74 | 0.000334 |
| GNG12 | 2.72 | 0.003946 |
| FDPS | 2.72 | 0.013411 |
| DSG1 | 2.72 | 3.89E−07 |
| YWHAQ | 2.71 | 0.001864 |
| IGKV1-16 | 2.69 | 0.006457 |
| LAMP1 | 2.69 | 0.003596 |
| ENG | 2.68 | 0.000983 |
| TPM3 | 2.67 | 1.31E−06 |
| MYO3A | 2.67 | 0.022487 |
| CAPN1 | 2.67 | 0.004755 |
| MAMDC2 | 2.64 | 0.014581 |
| MYH13 | 2.63 | 0.010167 |
| CCDC110 | 2.61 | 0.025959 |
| UNC13D | 2.61 | 6.39E−05 |
| AZGP1 | 2.59 | 8.12E−08 |
| IGLV7-46 | 2.59 | 2.12E−05 |
| MFAP2 | 2.58 | 0.000225 |
| KRAS | 2.57 | 0.000972 |
| ESD | 2.57 | 0.005135 |
| DSTN | 2.54 | 0.006875 |
| BST1 | 2.52 | 0.018602 |
| CNTFR | 2.51 | 0.006624 |
| IGHV1-46 | 2.50 | 1.45E−05 |
| MYLK | 2.49 | 0.000117 |
| H2AC21 | 2.47 | 0.001132 |
| HSP90AA1 | 2.45 | 1.04E−08 |
| COL9A1 | 2.44 | 0.003595 |
| ARPC1B | 2.44 | 0.002084 |
| TGM2 | 2.43 | 0.00205 |
| SLC44A2 | 2.43 | 0.012886 |
| TPP1 | 2.41 | 0.002061 |
| EPB41L1 | 2.39 | 0.026857 |
| PACSIN2 | 2.38 | 3.55E−05 |
| CCNB3 | 2.36 | 0.001287 |
| FHL1 | 2.36 | 0.002402 |
| GP9 | 2.35 | 0.003498 |
| SDC4 | 2.35 | 6.41E−05 |
| COP1 | 2.33 | 0.000435 |
| S100A13 | 2.32 | 0.00412 |
| GMPR | 2.32 | 0.006239 |
| RAB8B | 2.31 | 0.001874 |
| CKM | 2.31 | 0.011772 |
| TMC8 | 2.31 | 0.026692 |
| RAC2 | 2.27 | 3.6E−10 |
| F13A1 | 2.26 | 2.5E−07 |
| CD34 | 2.25 | 0.002686 |
| PLOD1 | 2.25 | 0.000714 |
| ARHGAP1 | 2.24 | 0.009884 |
| CCT7 | 2.24 | 0.007496 |
| LRRC59 | 2.23 | 0.006427 |
| GNB1 | 2.21 | 8.13E−05 |
| TSPAN33 | 2.21 | 0.014502 |
| TUBA8 | 2.20 | 0.00438 |
| GDI2 | 2.20 | 0.001214 |
| GPX1 | 2.19 | 4.02E−05 |
| UBE2D3 | 2.19 | 0.019397 |
| AGRN | 2.19 | 1.05E−05 |
| HIP1 | 2.18 | 0.013348 |
| DNAH14 | 2.18 | 0.034721 |
| PTPRJ | 2.17 | 0.010632 |
| EPB41L3 | 2.17 | 0.004269 |
| KIT | 2.17 | 1.47E−07 |
| EEF1G | 2.16 | 0.001644 |
| COMP | 2.15 | 0.000843 |
| COPS5 | 2.15 | 0.006709 |
| CROCC | 2.14 | 0.017985 |
| PDGFRB | 2.14 | 0.024622 |
| MARCKS | 2.13 | 9.55E−05 |
| SEPTIN7 | 2.12 | 0.029813 |
| TRIM7 | 2.11 | 0.017447 |
| MPP1 | 2.11 | 0.028828 |
| ARF3 | 2.11 | 4.97E−05 |
| PEBP1 | 2.11 | 7.82E−05 |
| RPL4 | 2.11 | 0.015036 |
| CD81 | 2.11 | 0.022106 |
| UTRN | 2.10 | 0.013306 |
| PARVB | 2.07 | 3.93E−06 |
| UBA1 | 2.07 | 0.00192 |
| FLT1 | 2.07 | 0.000237 |
| FGA | 2.06 | 3.05E−08 |
| STX11 | 2.05 | 0.005538 |
| SYMPK | 2.05 | 0.029327 |
| RPS4X | 2.05 | 0.000531 |
| ACTN4 | 2.03 | 0.000159 |
| ENO1 | 2.03 | 1.17E−07 |
| RPL13 | 2.02 | 0.036252 |
| TGFB1 | 2.02 | 0.010422 |
| IGKV3D-15 | 2.01 | 0.03058 |
| MTHFD1 | 1.98 | 0.033147 |
| PDCD6IP | 1.98 | 9.8E−05 |
| LOXL2 | 1.98 | 0.002378 |
| RALA | 1.97 | 0.030452 |
| ITGB1 | 1.96 | 8E−11 |
| LAMC2 | 1.95 | 0.000129 |
| VASN | 1.95 | 1.91E−06 |
| CAPZA2 | 1.95 | 0.001251 |
| IDE | 1.95 | 6.78E−05 |
| EIF5A | 1.95 | 0.000575 |
| ACTR2 | 1.94 | 6.37E−07 |
| RPL14 | 1.94 | 0.001723 |
| LAP3 | 1.94 | 5.36E−05 |
| PLAA | 1.92 | 0.003494 |
| CYFIP1 | 1.92 | 0.025712 |
| CAMP | 1.91 | 0.023111 |
| UBE2L3 | 1.90 | 0.011389 |
| ZNF800 | 1.90 | 0.022228 |
| RPS25 | 1.90 | 0.00532 |
| RPL11 | 1.89 | 0.012891 |
| CD63 | 1.88 | 0.001159 |
| IGFALS | 1.88 | 1.01E−05 |
| IGHV3-20 | 1.87 | 0.002723 |
| YWHAZ | 1.86 | 0.001333 |
| SAR1A | 1.85 | 0.003235 |
| CALU | 1.85 | 0.000369 |
| DNAJB2 | 1.84 | 0.016971 |
| GAPDH | 1.84 | 7.75E−06 |
| EGFR | 1.83 | 9.58E−05 |
| IGKV6D-21 | 1.80 | 0.017376 |
| ITGA3 | 1.80 | 2.31E−09 |
| KRT16 | 1.80 | 0.006106 |
| IGLV8-61 | 1.76 | 0.00018 |
| CAPNS1 | 1.75 | 0.016719 |
| RPS3 | 1.74 | 0.005878 |
| NT5E | 1.74 | 0.013852 |
| PKM | 1.71 | 0.002702 |
| FLNA | 1.70 | 5.52E−07 |
| TUBB3 | 1.70 | 0.017002 |
| ANXA7 | 1.66 | 0.004786 |
| IGHV2-5 | 1.66 | 0.001333 |
| HRNR | 1.65 | 0.00186 |
| RPS15A | 1.65 | 0.003786 |
| ARF6 | 1.62 | 0.005927 |
| PDIA3 | 1.62 | 0.001716 |
| H2BC15 | 1.61 | 0.000244 |
| FUCA1 | 1.60 | 8.36E−06 |
| C1QA | 1.60 | 0.00089 |
| GLIPR2 | 1.60 | 0.000144 |
| DDX55 | 1.59 | 0.035084 |
| PDLIM7 | 1.59 | 1.72E−05 |
| SERPINE1 | 1.59 | 0.00105 |
| CALM3 | 1.59 | 0.026876 |
| NPTX1 | 1.58 | 0.023521 |
| NIBAN2 | 1.58 | 0.007383 |
| PPBP | 1.57 | 0.000391 |
| HK1 | 1.57 | 0.031509 |
| FCN3 | 1.57 | 0.000268 |
| MYL6 | 1.57 | 0.000755 |
| PTGES3 | 1.56 | 0.023852 |
| GPR179 | 1.55 | 0.004002 |
| PRDX6 | 1.55 | 7.44E−05 |
| VCAN | 1.54 | 0.003195 |
| MSN | 1.54 | 1.34E−05 |
| C1RL | 1.52 | 4.62E−07 |
| RAB8A | 1.52 | 0.000118 |
| HTRA1 | 1.51 | 0.027976 |
| C1QB | 1.51 | 0.000215 |
| S100A4 | 1.51 | 0.034502 |
| IGHV3-64D | 1.51 | 0.001005 |
| DTD1 | 1.50 | 0.007098 |
| THBS2 | 1.50 | 0.023918 |
| PATJ | 1.50 | 5.49E−05 |
| CFH | 1.50 | 0.00065 |
| HSPA5 | 1.49 | 2.24E−05 |
| UBA52 | 1.49 | 0.006603 |
| HLA-A | 1.49 | 0.027277 |
| IGKV3-7 | 1.49 | 6.84E−05 |
| RAP2A | 1.48 | 0.01951 |
| CNTNAP5 | 1.48 | 0.020994 |
| APOA1 | 1.47 | 7.05E−05 |
| CD59 | 1.46 | 0.017075 |
| TGFBI | 1.46 | 0.000145 |
| EHD3 | 1.45 | 6.79E−07 |
| TMTC2 | 1.45 | 0.000856 |
| CD276 | 1.45 | 0.003687 |
| IGLV3-21 | 1.45 | 6.09E−05 |
| PLXDC2 | 1.43 | 2.54E−06 |
| SP5 | 1.43 | 0.033692 |
| AHCY | 1.42 | 0.010938 |
| IGHG3 | 1.41 | 0.003373 |
| PTPRG | 1.41 | 5.65E−06 |
| SERPINC1 | 1.40 | 1.47E−05 |
| C1R | 1.40 | 0.000988 |
| HABP2 | 1.40 | 0.000584 |
| FN1 | 1.40 | 0.0248 |
| C1S | 1.40 | 2.03E−05 |
| FBN1 | 1.38 | 5.52E−05 |
| CDC42 | 1.38 | 1.69E−05 |
| INF2 | 1.37 | 0.007261 |
| HBA1 | 1.37 | 2.11E−05 |
| PCYOX1 | 1.37 | 0.015589 |
| HBD | 1.37 | 0.00225 |
| SELENOP | 1.37 | 3.88E−05 |
| C8B | 1.36 | 0.000264 |
| C9 | 1.36 | 1.91E−05 |
| TUBB1 | 1.36 | 1.06E−05 |
| PI16 | 1.35 | 0.000586 |
| EMILIN1 | 1.35 | 0.027038 |
| LYN | 1.35 | 0.008809 |
| VPS13A | 1.33 | 0.001477 |
| IGLV1-47 | 1.32 | 0.001108 |
| COTL1 | 1.31 | 0.018812 |
| CLTC | 1.31 | 0.00392 |
| IGHV3-33 | 1.31 | 0.005984 |
| CPB2 | 1.30 | 1.11E−06 |
| F12 | 1.30 | 8.18E−05 |
| TUBA1B | 1.30 | 3.08E−09 |
| IGLV4-69 | 1.29 | 0.000486 |
| RAB7A | 1.29 | 0.016284 |
| NAA25 | 1.28 | 0.001563 |
| F2 | 1.28 | 0.000124 |
| CLEC3B | 1.28 | 0.01992 |
| C1QC | 1.25 | 0.000596 |
| APP | 1.24 | 0.001311 |
| SERPINA1 | 1.23 | 2.47E−07 |
| DENND2A | 1.21 | 0.033105 |
| GSTP1 | 1.20 | 4.84E−07 |
| NID2 | 1.20 | 0.014748 |
| RNASE11 | 1.19 | 0.03571 |
| COL6A2 | 1.19 | 0.020663 |
| NUTF2 | 1.19 | 0.000436 |
| YWHAG | 1.18 | 9.65E−06 |
| PEPD | 1.18 | 3.67E−06 |
| PPP1CA | 1.17 | 0.017239 |
| ILK | 1.16 | 0.022235 |
| EHD1 | 1.15 | 0.001047 |
| APCS | 1.15 | 1.05E−05 |
| RALB | 1.15 | 4.1E−05 |
| IGHV3-73 | 1.14 | 0.00507 |
| IGHA2 | 1.13 | 0.000613 |
| CD36 | 1.12 | 8E−07 |
| HRG | 1.12 | 9.25E−05 |
| GALE | 1.12 | 0.034543 |
| VASP | 1.11 | 0.002503 |
| ACE | 1.09 | 4.32E−05 |
| TUBB | 1.09 | 0.000838 |
| TPI1 | 1.09 | 3.71E−05 |
| RAC1 | 1.07 | 0.000549 |
| ANXA2 | 1.07 | 8.86E−08 |
| FAH | 1.07 | 0.023699 |
| TUBB4B | 1.06 | 4.98E−06 |
| GSN | 1.06 | 2.88E−06 |
| EIF4A1 | 1.05 | 4.7E−06 |
| COL5A1 | 1.04 | 5.41E−05 |
| FERMT3 | 1.03 | 4.1E−07 |
| ITGA2B | 1.03 | 0.001339 |
| PROS1 | 1.02 | 0.002371 |
| HSP90B1 | 1.00 | 0.015352 |
| LGALS3BP | 1.00 | 0.003375 |

TABLE 18
Proteins significantly more abundant
in BM-MSC EVs compared to HMC-EVs

| Name | log 2 fold difference | p-value |
|---|---|---|
| DMXL1 | −9.78 | 4.91E−13 |
| PXYLP1 | −7.74 | 1.34E−08 |
| PTGFRN | −7.55 | 1.38E−14 |
| CSHL1 | −4.51 | 6.52E−03 |
| RNH1 | −4.24 | 3.53E−02 |
| AASS | −3.91 | 7.86E−03 |
| APOL1 | −3.85 | 4.66E−12 |
| RPL15 | −3.72 | 8.13E−03 |
| IRF6 | −3.48 | 1.71E−02 |
| TMEM198 | −3.29 | 1.49E−03 |
| RAB1B | −3.10 | 8.69E−03 |
| ASPM | −2.91 | 2.21E−02 |
| SULT1A1 | −2.79 | 1.21E−08 |
| GP5 | −2.69 | 5.64E−08 |
| CAT | −2.63 | 3.53E−02 |
| KYAT3 | −2.54 | 6.63E−06 |
| CCT2 | −2.46 | 1.87E−07 |
| TAS2R33 | −2.41 | 3.11E−03 |
| FGG | −2.33 | 3.66E−10 |
| ABI3BP | −2.30 | 7.93E−08 |
| ARMCX5 | −2.25 | 2.54E−02 |
| IGLV6-57 | −2.23 | 2.66E−03 |
| ADIPOQ | −2.23 | 2.36E−02 |
| WNT5B | −2.23 | 2.45E−02 |
| IGKV1D-39 | −2.18 | 1.61E−05 |
| CUX1 | −2.10 | 1.19E−02 |
| LILRA3 | −2.06 | 2.74E−03 |
| PPM1F | −2.01 | 1.70E−02 |
| GM2A | −2.01 | 1.32E−02 |
| CEP290 | −2.01 | 2.08E−02 |
| IGLV3-1 | −2.01 | 6.15E−03 |
| CTSK | −1.94 | 1.70E−02 |
| IGHV3-38 | −1.78 | 1.08E−03 |
| CCDC80 | −1.77 | 1.88E−02 |
| DOCK9 | −1.72 | 1.45E−04 |
| LAMA4 | −1.71 | 1.52E−02 |
| NAP1L4 | −1.60 | 1.91E−04 |
| APOA2 | −1.59 | 3.14E−04 |
| NBEAL2 | −1.58 | 2.65E−02 |
| KRT81 | −1.48 | 2.99E−02 |
| AASDHPPT | −1.46 | 1.29E−02 |
| PAICS | −1.45 | 2.47E−06 |
| FBLN5 | −1.45 | 6.53E−04 |
| MUC16 | −1.44 | 3.66E−02 |
| PRXL2C | −1.42 | 1.97E−05 |
| IGLV4-60 | −1.40 | 3.26E−02 |
| AKR7A2 | −1.39 | 2.67E−04 |
| SRR | −1.30 | 1.57E−02 |
| CYLC2 | −1.26 | 1.37E−05 |
| COL3A1 | −1.20 | 3.78E−03 |
| GMFG | −1.19 | 2.10E−02 |
| PDLIM1 | −1.16 | 8.70E−05 |
| SPOCK1 | −1.04 | 1.69E−04 |
| ITIH1 | −1.00 | 1.51E−02 |

TABLE 19
Proteins significantly more abundant
in HMC-EVs compared to AD-MSC EVs

| Name | log 2 fold difference | P-Value |
|---|---|---|
| SEPTIN5 | 6.88 | 3.98E−08 |
| B2M | 6.71 | 1.29E−07 |
| H3-3A | 6.61 | 9.60E−04 |
| PRSS23 | 6.46 | 1.73E−08 |
| SLC2A1 | 5.40 | 1.62E−07 |
| IGKV3D-20 | 5.14 | 9.44E−05 |
| RAB6B | 5.13 | 2.00E−04 |
| APBB2 | 5.11 | 1.32E−06 |
| LTBP3 | 5.01 | 3.28E−03 |
| PGAP1 | 5.00 | 1.75E−04 |
| TAGLN2 | 4.69 | 1.16E−10 |
| CD81 | 4.64 | 6.54E−07 |
| SRSF8 | 4.55 | 3.60E−05 |
| BSG | 4.54 | 1.65E−02 |
| ENG | 4.52 | 6.54E−05 |
| NT5E | 4.49 | 2.13E−03 |
| RPS3A | 4.39 | 1.21E−06 |
| S100A11 | 4.38 | 3.93E−05 |
| CA2 | 4.30 | 8.95E−04 |
| CD99 | 4.29 | 2.10E−07 |
| ESD | 4.24 | 1.58E−03 |
| TSPAN14 | 4.20 | 7.38E−05 |
| RPS4X | 4.13 | 1.15E−04 |
| CAV1 | 4.13 | 1.15E−05 |
| FSCN1 | 4.12 | 2.04E−02 |
| ARF4 | 4.10 | 2.53E−03 |
| ITGA2 | 4.10 | 4.84E−05 |
| ANXA5 | 4.08 | 1.59E−04 |
| RPS18 | 4.07 | 1.04E−03 |
| BLVRA | 4.07 | 2.14E−03 |
| VAT1 | 4.06 | 1.27E−04 |
| MAMDC2 | 4.05 | 3.48E−03 |
| KIF11 | 4.00 | 8.93E−03 |
| GNAQ | 3.99 | 4.47E−05 |
| CKM | 3.99 | 2.49E−03 |
| YWHAQ | 3.99 | 1.25E−02 |
| CD36 | 3.99 | 8.04E−04 |
| MARCKSL1 | 3.94 | 2.00E−05 |
| ARHGDIB | 3.90 | 8.12E−03 |
| RAB27B | 3.89 | 2.15E−03 |
| GNAI2 | 3.88 | 3.08E−03 |
| H3-7 | 3.85 | 2.02E−03 |
| KRAS | 3.77 | 1.12E−03 |
| ARHGDIA | 3.69 | 3.75E−03 |
| MFGE8 | 3.63 | 1.01E−07 |
| MEIOB | 3.63 | 3.25E−03 |
| CDC42 | 3.63 | 6.84E−05 |
| SH3BGRL3 | 3.62 | 2.31E−03 |
| STXBP2 | 3.59 | 4.95E−04 |
| STX11 | 3.58 | 8.41E−05 |
| ARL8A | 3.56 | 2.66E−06 |
| TRPM2 | 3.53 | 6.82E−04 |
| CCN2 | 3.52 | 4.52E−04 |
| H2BC15 | 3.48 | 3.02E−07 |
| MERTK | 3.46 | 2.22E−03 |
| YWHAB | 3.46 | 8.75E−03 |
| ALDOC | 3.44 | 1.72E−07 |
| TUBB3 | 3.44 | 2.51E−03 |
| FDPS | 3.40 | 2.12E−03 |
| SFRP1 | 3.39 | 1.70E−03 |
| TSPAN33 | 3.39 | 1.54E−04 |
| PCDH1 | 3.38 | 1.50E−02 |
| MBTD1 | 3.37 | 3.98E−02 |
| SLTM | 3.37 | 8.65E−03 |
| COL4A2 | 3.35 | 1.76E−06 |
| MARCKS | 3.34 | 5.03E−06 |
| FUCA1 | 3.34 | 5.42E−08 |
| TSPAN9 | 3.33 | 3.04E−04 |
| CD47 | 3.31 | 1.77E−04 |
| DTD1 | 3.29 | 4.19E−05 |
| KPNA2 | 3.29 | 5.32E−05 |
| MDGA1 | 3.27 | 5.41E−04 |
| BCL9 | 3.24 | 6.32E−03 |
| HIP1 | 3.23 | 2.83E−03 |
| IGLV2-23 | 3.23 | 2.68E−02 |
| TTYH3 | 3.23 | 1.38E−04 |
| TNC | 3.22 | 4.36E−02 |
| LAMP1 | 3.21 | 3.34E−03 |
| HLA-A | 3.18 | 1.74E−02 |
| PPP2R1A | 3.18 | 2.11E−03 |
| MDH1 | 3.17 | 3.10E−04 |
| MYO3A | 3.14 | 1.89E−02 |
| PGD | 3.13 | 3.38E−02 |
| RPS12 | 3.12 | 4.41E−04 |
| PXDN | 3.11 | 5.32E−06 |
| YWHAE | 3.10 | 1.50E−03 |
| PRXL2C | 3.10 | 1.99E−02 |
| GNG12 | 3.10 | 1.59E−03 |
| ARPC5 | 3.08 | 4.69E−03 |
| LRRC59 | 3.05 | 2.84E−03 |
| PF4 | 3.05 | 2.00E−06 |
| SLC44A1 | 3.05 | 1.83E−07 |
| TPI1 | 3.03 | 6.86E−06 |
| CCNB3 | 3.03 | 1.22E−03 |
| CD63 | 3.02 | 1.40E−05 |
| GP9 | 3.01 | 5.73E−04 |
| PSTPIP2 | 2.99 | 5.92E−06 |
| HP | 2.97 | 8.61E−04 |
| PPP2CB | 2.96 | 8.89E−03 |
| H2AC20 | 2.96 | 1.77E−02 |
| BST1 | 2.96 | 1.45E−02 |
| SLC3A2 | 2.94 | 4.64E−02 |
| ACACA | 2.94 | 4.53E−03 |
| MTPN | 2.93 | 1.85E−02 |
| EPB41L3 | 2.93 | 2.63E−03 |
| MMP14 | 2.89 | 2.18E−05 |
| RPS3 | 2.87 | 1.26E−03 |
| BMP1 | 2.87 | 3.15E−02 |
| FCGR3A | 2.86 | 4.72E−03 |
| COP1 | 2.86 | 3.04E−02 |
| UCHL1 | 2.85 | 1.26E−05 |
| PEBP1 | 2.85 | 6.66E−04 |
| SLC44A2 | 2.85 | 5.32E−03 |
| SDCBP | 2.82 | 1.40E−06 |
| PLXDC2 | 2.81 | 2.13E−04 |
| RAB11FIP1 | 2.80 | 1.23E−04 |
| RNASE11 | 2.80 | 9.39E−03 |
| MYL12B | 2.77 | 1.88E−03 |
| RPL10A | 2.76 | 3.20E−05 |
| SMG1 | 2.75 | 2.02E−08 |
| ITGA3 | 2.73 | 2.01E−05 |
| PDIA3 | 2.72 | 3.30E−03 |
| H4-16 | 2.71 | 5.73E−04 |
| AGRN | 2.71 | 3.35E−05 |
| AOC3 | 2.71 | 4.15E−04 |
| ARPC2 | 2.69 | 1.06E−02 |
| ITGA2B | 2.69 | 3.15E−06 |
| VCAN | 2.69 | 1.80E−05 |
| COTL1 | 2.67 | 1.88E−03 |
| RPL13 | 2.66 | 1.16E−02 |
| RICTOR | 2.64 | 1.71E−02 |
| DYNLL1 | 2.64 | 8.64E−04 |
| H1-3 | 2.63 | 1.03E−03 |
| H2AC21 | 2.63 | 1.64E−02 |
| TPP1 | 2.62 | 7.50E−03 |
| RAB14 | 2.61 | 4.68E−04 |
| PDGFRB | 2.61 | 2.90E−02 |
| RPL14 | 2.61 | 3.28E−05 |
| TUBA8 | 2.59 | 4.67E−03 |
| ADGRG6 | 2.59 | 4.04E−03 |
| KRT4 | 2.58 | 7.61E−03 |
| MYO1F | 2.57 | 1.00E−02 |
| EPHB2 | 2.56 | 1.62E−03 |
| RALA | 2.56 | 4.82E−04 |
| RPS25 | 2.55 | 1.54E−02 |
| MAPK3 | 2.55 | 7.18E−03 |
| STOM | 2.53 | 4.63E−05 |
| CTBS | 2.51 | 6.65E−05 |
| SERPINA10 | 2.50 | 1.92E−02 |
| GNB4 | 2.50 | 2.35E−02 |
| ANXA6 | 2.49 | 1.01E−05 |
| CD276 | 2.49 | 2.99E−03 |
| GLIPR2 | 2.49 | 6.02E−05 |
| BBS1 | 2.48 | 2.48E−02 |
| BASP1 | 2.48 | 4.27E−02 |
| MVP | 2.47 | 3.35E−02 |
| FAH | 2.47 | 8.09E−03 |
| CD34 | 2.47 | 2.04E−03 |
| NAGLU | 2.47 | 4.76E−04 |
| PTPRG | 2.47 | 1.09E−03 |
| THY1 | 2.45 | 1.37E−06 |
| PRG4 | 2.42 | 8.78E−06 |
| RPS15A | 2.42 | 1.07E−02 |
| FREM3 | 2.42 | 1.08E−04 |
| MOB1B | 2.42 | 9.08E−03 |
| FLG2 | 2.42 | 2.52E−02 |
| SEPTIN2 | 2.39 | 1.29E−04 |
| PTGDS | 2.38 | 1.39E−03 |
| IL1RAP | 2.38 | 4.95E−04 |
| NIBAN2 | 2.38 | 2.82E−04 |
| LGALS1 | 2.37 | 1.45E−06 |
| GSTM1 | 2.37 | 6.30E−03 |
| EEF1D | 2.36 | 3.60E−06 |
| SPARC | 2.35 | 3.57E−02 |
| UBE2L3 | 2.34 | 5.48E−03 |
| CBR1 | 2.34 | 4.64E−02 |
| RAP2A | 2.34 | 7.67E−03 |
| TANK | 2.34 | 1.89E−05 |
| S100A6 | 2.32 | 5.70E−03 |
| CRISP3 | 2.29 | 3.53E−05 |
| ANXA2 | 2.28 | 2.47E−07 |
| MON2 | 2.28 | 6.21E−06 |
| APOC4 | 2.28 | 4.38E−04 |
| MTHFD1 | 2.27 | 3.08E−02 |
| DEFA3 | 2.27 | 5.03E−03 |
| NPM1 | 2.26 | 1.08E−02 |
| C1QA | 2.25 | 5.27E−05 |
| ACLY | 2.24 | 3.88E−02 |
| ITGB3 | 2.24 | 4.84E−07 |
| CPN2 | 2.23 | 5.77E−08 |
| RPS13 | 2.22 | 3.10E−03 |
| ARHGAP1 | 2.20 | 4.63E−02 |
| HYOU1 | 2.20 | 2.88E−02 |
| IGLV7-43 | 2.19 | 3.58E−02 |
| GNB1 | 2.19 | 2.91E−05 |
| ZNF607 | 2.19 | 7.60E−04 |
| TGM2 | 2.18 | 9.01E−03 |
| CORO1A | 2.18 | 4.53E−05 |
| CD9 | 2.18 | 2.71E−06 |
| STRADB | 2.16 | 3.17E−02 |
| GATA5 | 2.16 | 3.73E−05 |
| YBX3 | 2.16 | 4.51E−02 |
| EHD1 | 2.15 | 2.63E−03 |
| LUM | 2.13 | 5.69E−05 |
| CNDP2 | 2.12 | 3.00E−03 |
| ITGA4 | 2.10 | 5.79E−04 |
| RNF149 | 2.09 | 2.50E−04 |
| SRPX | 2.09 | 1.32E−09 |
| HSP90AB1 | 2.09 | 4.04E−05 |
| LAP3 | 2.08 | 4.83E−04 |
| ITGB1 | 2.05 | 1.72E−07 |
| HSPA4L | 2.05 | 1.89E−04 |
| TPTE2 | 2.05 | 1.85E−02 |
| QSOX1 | 2.04 | 3.25E−04 |
| PLOD1 | 2.04 | 5.58E−02 |
| SERPINA11 | 2.04 | 1.85E−02 |
| EEF1G | 2.02 | 8.25E−03 |
| DENND2A | 2.01 | 5.43E−03 |
| RPSA | 2.01 | 7.73E−06 |
| PRKAR1A | 2.01 | 3.58E−03 |
| LCAT | 2.01 | 9.99E−08 |
| C1QB | 2.00 | 1.60E−05 |
| PROCR | 2.00 | 4.29E−06 |
| MYH13 | 1.98 | 1.77E−02 |
| NME2 | 1.95 | 1.43E−03 |
| PGLYRP2 | 1.95 | 7.91E−09 |
| SDC4 | 1.94 | 2.60E−03 |
| PTPN6 | 1.94 | 2.68E−04 |
| C1RL | 1.93 | 1.91E−04 |
| AFM | 1.92 | 4.17E−06 |
| B4GALT1 | 1.92 | 9.83E−03 |
| CNTFR | 1.92 | 4.02E−04 |
| HSPE1-MOB4 | 1.92 | 3.09E−02 |
| COL18A1 | 1.91 | 1.23E−02 |
| ARF6 | 1.91 | 7.99E−03 |
| ACOT7 | 1.91 | 4.52E−04 |
| ROBO4 | 1.90 | 1.22E−03 |
| CAPZA2 | 1.90 | 5.23E−02 |
| CLIC4 | 1.90 | 3.52E−03 |
| RAB8B | 1.89 | 1.57E−02 |
| PFN1 | 1.89 | 5.10E−06 |
| APRT | 1.88 | 1.18E−02 |
| RBP4 | 1.87 | 4.24E−04 |
| ACTR3 | 1.87 | 2.56E−02 |
| MYL6 | 1.86 | 2.32E−04 |
| CD82 | 1.86 | 1.27E−03 |
| PDCD6IP | 1.86 | 3.88E−06 |
| ARHGAP6 | 1.86 | 2.73E−02 |
| ADCY5 | 1.86 | 1.09E−04 |
| SDC1 | 1.85 | 3.15E−05 |
| C1QC | 1.85 | 1.71E−04 |
| ITGA6 | 1.82 | 6.75E−06 |
| GMPR | 1.82 | 2.99E−02 |
| VNN1 | 1.82 | 2.85E−05 |
| TUBA4A | 1.82 | 7.14E−03 |
| GPNMB | 1.81 | 1.32E−02 |
| GGH | 1.81 | 6.85E−05 |
| NUTF2 | 1.80 | 6.20E−04 |
| CDH5 | 1.79 | 3.11E−04 |
| INF2 | 1.79 | 2.52E−02 |
| OSMR | 1.78 | 2.41E−04 |
| AHSG | 1.77 | 2.67E−07 |
| RPL4 | 1.76 | 2.48E−02 |
| PDE4DIP | 1.75 | 1.35E−07 |
| RALB | 1.74 | 1.20E−05 |
| TBC1D2 | 1.74 | 2.92E−02 |
| EHD3 | 1.73 | 1.45E−04 |
| EIF3K | 1.73 | 1.38E−02 |
| C1R | 1.73 | 5.18E−05 |
| IGHG4 | 1.71 | 3.10E−04 |
| LGALSL | 1.71 | 1.49E−06 |
| LIPT1 | 1.71 | 4.62E−02 |
| WDR48 | 1.70 | 4.83E−05 |
| FARP1 | 1.70 | 8.11E−03 |
| RAB11B | 1.69 | 2.80E−02 |
| UNC13D | 1.69 | 2.60E−02 |
| PAFAH1B1 | 1.69 | 9.85E−04 |
| IGKV6D-21 | 1.68 | 5.48E−04 |
| ARPC1B | 1.67 | 3.51E−03 |
| LCP2 | 1.67 | 9.04E−03 |
| TUBB1 | 1.67 | 5.82E−04 |
| CDH13 | 1.64 | 3.23E−03 |
| AHCY | 1.64 | 1.06E−02 |
| SLC22A23 | 1.64 | 3.11E−04 |
| GANAB | 1.63 | 3.25E−06 |
| SELL | 1.63 | 2.53E−07 |
| PRPH2 | 1.63 | 2.30E−05 |
| PYGB | 1.62 | 1.12E−04 |
| CLIC1 | 1.62 | 3.58E−04 |
| MYO15A | 1.60 | 1.45E−04 |
| TMC8 | 1.60 | 1.06E−02 |
| LOXL2 | 1.60 | 8.00E−04 |
| APOE | 1.58 | 3.45E−04 |
| RPL11 | 1.57 | 1.32E−02 |
| RAP1B | 1.57 | 2.29E−03 |
| FGA | 1.56 | 8.45E−05 |
| RAB8A | 1.56 | 4.90E−03 |
| GSTO1 | 1.56 | 1.40E−03 |
| LRG1 | 1.55 | 1.69E−04 |
| UBA52 | 1.54 | 7.44E−05 |
| HLA-A | 1.54 | 4.79E−02 |
| CD14 | 1.54 | 3.09E−04 |
| CALM3 | 1.53 | 1.38E−04 |
| RHOA | 1.53 | 8.42E−05 |
| ITGA5 | 1.51 | 2.00E−06 |
| HPX | 1.51 | 5.50E−05 |
| APOA2 | 1.50 | 2.40E−02 |
| NEBL | 1.50 | 6.28E−03 |
| CCT4 | 1.50 | 1.65E−04 |
| LRP1 | 1.49 | 1.91E−04 |
| TEX35 | 1.49 | 1.11E−04 |
| ARPC4 | 1.48 | 2.63E−04 |
| LPA | 1.48 | 7.69E−05 |
| OBSCN | 1.47 | 3.86E−03 |
| ACE | 1.47 | 6.26E−03 |
| CALR | 1.46 | 9.54E−03 |
| HP | 1.46 | 4.00E−04 |
| TUBB4B | 1.45 | 4.15E−07 |
| MAPRE2 | 1.45 | 6.84E−04 |
| ILK | 1.43 | 1.31E−06 |
| LAMC2 | 1.43 | 1.05E−02 |
| YWHAG | 1.43 | 1.25E−03 |
| SERPINA6 | 1.42 | 8.01E−07 |
| FUCA2 | 1.41 | 3.97E−04 |
| PCOLCE | 1.41 | 2.17E−03 |
| POTEJ | 1.40 | 9.12E−04 |
| MCAM | 1.40 | 6.43E−05 |
| MYH9 | 1.40 | 1.46E−05 |
| LBP | 1.40 | 2.72E−03 |
| DSTN | 1.38 | 7.18E−05 |
| DYNC1H1 | 1.38 | 1.11E−05 |
| YWHAZ | 1.38 | 1.22E−06 |
| FERMT3 | 1.37 | 6.62E−06 |
| PPIA | 1.37 | 6.55E−05 |
| APMAP | 1.35 | 1.77E−02 |
| PI16 | 1.34 | 7.66E−07 |
| A1BG | 1.33 | 5.52E−08 |
| DNAJB2 | 1.33 | 2.80E−02 |
| EDIL3 | 1.33 | 2.07E−03 |
| PSMB4 | 1.33 | 3.52E−02 |
| APP | 1.33 | 4.69E−05 |
| CAP1 | 1.33 | 1.24E−04 |
| PRKDC | 1.31 | 1.33E−04 |
| CACNA2D1 | 1.31 | 1.64E−03 |
| SYK | 1.31 | 5.47E−03 |
| AKR7A2 | 1.30 | 2.85E−04 |
| COL1A2 | 1.29 | 4.13E−03 |
| FN1 | 1.29 | 2.43E−03 |
| ZNF879 | 1.29 | 1.30E−02 |
| RAB10 | 1.27 | 1.34E−04 |
| SRC | 1.26 | 2.09E−03 |
| PVR | 1.26 | 2.61E−03 |
| APCS | 1.26 | 1.19E−04 |
| WARS1 | 1.26 | 5.38E−02 |
| CNN2 | 1.25 | 2.34E−02 |
| PKM | 1.25 | 1.66E−03 |
| PFKP | 1.25 | 2.58E−04 |
| GAPDH | 1.25 | 3.31E−03 |
| IGFALS | 1.24 | 2.59E−02 |
| ALDOA | 1.23 | 4.07E−05 |
| BCHE | 1.23 | 1.05E−04 |
| ALOX12 | 1.23 | 4.37E−02 |
| HSPB1 | 1.23 | 9.90E−04 |
| CD59 | 1.23 | 4.25E−04 |
| CSF1R | 1.22 | 3.24E−03 |
| PRDX6 | 1.22 | 1.01E−03 |
| MIF | 1.22 | 2.05E−04 |
| COL6A2 | 1.22 | 6.03E−06 |
| MTAP | 1.21 | 5.61E−03 |
| COL6A3 | 1.21 | 1.03E−04 |
| F10 | 1.21 | 1.34E−03 |
| BANF1 | 1.21 | 8.40E−04 |
| F13A1 | 1.19 | 1.39E−04 |
| APOA4 | 1.19 | 9.01E−06 |
| FGG | 1.18 | 6.87E−07 |
| SAR1A | 1.17 | 9.11E−03 |
| ARPC3 | 1.17 | 7.77E−04 |
| ADAMTS12 | 1.16 | 4.03E−02 |
| EEF2 | 1.15 | 7.16E−05 |
| VTN | 1.15 | 5.04E−04 |
| C1S | 1.15 | 1.22E−06 |
| CETP | 1.15 | 4.16E−03 |
| ADH5 | 1.14 | 5.04E−03 |
| HABP2 | 1.14 | 2.65E−02 |
| SYNE1 | 1.13 | 5.60E−04 |
| TIMM13 | 1.13 | 6.67E−05 |
| APOC4-APOC2 | 1.13 | 2.40E−02 |
| APOC3 | 1.13 | 1.88E−02 |
| HP | 1.13 | 1.14E−04 |
| SPP2 | 1.12 | 2.41E−05 |
| PPBP | 1.12 | 2.83E−05 |
| CC2D2B | 1.12 | 1.48E−02 |
| COL1A1 | 1.12 | 8.40E−04 |
| AHNAK | 1.12 | 2.44E−03 |
| TPX2 | 1.11 | 4.00E−03 |
| FBN2 | 1.11 | 1.06E−02 |
| APOC1 | 1.11 | 1.22E−03 |
| IGHM | 1.11 | 1.29E−03 |
| MASP2 | 1.10 | 3.47E−03 |
| PGK1 | 1.10 | 1.05E−03 |
| DIAPH1 | 1.09 | 4.53E−02 |
| AGT | 1.09 | 4.53E−06 |
| CCT3 | 1.09 | 3.36E−02 |
| DPP4 | 1.08 | 1.78E−06 |
| CPB2 | 1.08 | 4.46E−03 |
| PEPD | 1.07 | 5.39E−09 |
| BGN | 1.07 | 6.47E−05 |
| IDE | 1.07 | 4.31E−05 |
| DNAJC12 | 1.06 | 1.42E−04 |
| PTGES3 | 1.06 | 4.09E−02 |
| APOH | 1.05 | 6.15E−06 |
| CCT2 | 1.05 | 9.47E−04 |
| ACTB | 1.05 | 6.98E−08 |
| MTA2 | 1.05 | 6.42E−03 |
| MRC2 | 1.05 | 1.19E−02 |
| TUBA1B | 1.03 | 2.86E−04 |
| CD5L | 1.02 | 3.55E−04 |
| CFHR1 | 1.02 | 1.07E−03 |
| CTSD | 1.01 | 1.49E−03 |
| FCGBP | 1.01 | 9.17E−04 |
| ARF3 | 1.00 | 6.70E−04 |
| CAPZB | 1.00 | 1.91E−03 |

TABLE 20
Proteins significantly more abundant
in AD-MSC EVs compared to HMC-EVs

| Name | log 2 fold difference | P-Value |
|---|---|---|
| TMEM198 | −6.91 | 6.10E−12 |
| ARMCX5 | −6.41 | 1.83E−02 |
| SH3BGRL | −4.93 | 2.45E−06 |
| CAT | −4.70 | 4.13E−07 |
| CEP290 | −4.35 | 2.28E−09 |
| TAS2R33 | −4.18 | 4.86E−03 |
| ALB | −4.13 | 3.11E−05 |
| KRT81 | −4.04 | 2.94E−07 |
| ADIPOQ | −3.99 | 3.32E−04 |
| SEMA7A | −3.95 | 5.59E−07 |
| SPON2 | −3.94 | 3.38E−04 |
| CHST9 | −3.93 | 2.60E−08 |
| IGHV1-45 | −3.90 | 7.40E−03 |
| CD109 | −3.78 | 2.49E−09 |
| NEO1 | −3.70 | 5.61E−06 |
| IQGAP2 | −3.68 | 4.59E−07 |
| SURF1 | −3.66 | 3.77E−09 |
| SEPTIN6 | −3.58 | 2.10E−08 |
| LTF | −3.57 | 6.82E−03 |
| ZNF800 | −3.56 | 8.17E−09 |
| ERC1 | −3.46 | 6.65E−05 |
| ITPR3 | −3.44 | 7.92E−07 |
| MSH6 | −3.37 | 2.76E−03 |
| OLFML3 | −3.30 | 7.36E−03 |
| ALB | −3.24 | 8.12E−06 |
| HAUS6 | −3.23 | 1.89E−03 |
| PAK6 | −3.16 | 2.33E−03 |
| PRDX2 | −3.16 | 1.33E−05 |
| AKAP9 | −3.12 | 5.02E−04 |
| HAUS8 | −3.11 | 9.58E−07 |
| ALOX5 | −3.08 | 9.38E−07 |
| PRKACB | −3.07 | 5.34E−03 |
| CDSN | −3.05 | 2.49E−03 |
| SLC9A4 | −3.04 | 1.49E−08 |
| LRRTM2 | −3.00 | 1.13E−04 |
| ALX4 | −2.99 | 1.46E−06 |
| GPR179 | −2.97 | 1.08E−07 |
| CYLC2 | −2.93 | 5.87E−07 |
| DSC1 | −2.91 | 2.21E−08 |
| NBEAL2 | −2.90 | 4.83E−05 |
| DDX55 | −2.78 | 7.01E−11 |
| SYMPK | −2.77 | 7.34E−08 |
| L1TD1 | −2.77 | 5.98E−08 |
| QDPR | −2.76 | 1.82E−04 |
| C6 | −2.76 | 2.19E−12 |
| RGS14 | −2.72 | 2.47E−03 |
| CNDP1 | −2.68 | 4.27E−10 |
| LRAT | −2.62 | 5.70E−03 |
| LAMB4 | −2.61 | 1.47E−04 |
| F11 | −2.59 | 9.76E−08 |
| RPS6KA4 | −2.55 | 7.87E−03 |
| MOGS | −2.51 | 3.62E−02 |
| IGLV6-57 | −2.50 | 6.55E−03 |
| CCDC178 | −2.50 | 9.07E−03 |
| ATP10A | −2.49 | 1.15E−04 |
| SLC24A4 | −2.49 | 7.62E−03 |
| PHF24 | −2.47 | 1.84E−05 |
| SNX14 | −2.44 | 3.37E−06 |
| DCN | −2.43 | 2.90E−04 |
| IGHV1-8 | −2.43 | 1.58E−02 |
| VCP | −2.41 | 9.84E−06 |
| OAF | −2.39 | 1.15E−03 |
| COG2 | −2.39 | 3.26E−02 |
| TRIM4 | −2.38 | 7.87E−03 |
| GTF2IRD2 | −2.37 | 1.81E−08 |
| TRIM7 | −2.35 | 4.33E−06 |
| NID2 | −2.33 | 3.79E−06 |
| RPL13A | −2.30 | 3.89E−02 |
| TNFAIP6 | −2.30 | 1.08E−05 |
| IGLL1 | −2.25 | 1.61E−03 |
| GMFG | −2.21 | 4.87E−03 |
| DBH | −2.21 | 3.65E−02 |
| SERPINB12 | −2.20 | 3.30E−02 |
| PSMA7 | −2.18 | 1.94E−08 |
| TIMP2 | −2.17 | 1.70E−03 |
| IGHV5-51 | −2.11 | 3.25E−07 |
| RACK1 | −2.09 | 8.66E−06 |
| APLP2 | −2.05 | 5.38E−03 |
| IGHV1-69D | −2.02 | 6.38E−03 |
| KRT16 | −2.02 | 1.24E−04 |
| IGHV2-26 | −2.00 | 7.32E−05 |
| CAPN5 | −2.00 | 6.65E−07 |
| PSMA6 | −1.97 | 5.32E−02 |
| IGHG1 | −1.97 | 1.83E−07 |
| IGHG2 | −1.95 | 2.21E−07 |
| CCT6A | −1.93 | 3.26E−05 |
| GP6 | −1.93 | 1.75E−02 |
| C18orf63 | −1.88 | 9.55E−06 |
| ANO7 | −1.88 | 3.89E−08 |
| IGLV4-60 | −1.87 | 3.29E−02 |
| XYLT1 | −1.84 | 3.86E−03 |
| FAM180A | −1.81 | 7.96E−04 |
| LYVE1 | −1.81 | 1.23E−02 |
| ERFL | −1.78 | 2.65E−09 |
| CRTAP | −1.78 | 1.15E−02 |
| MYCBP2 | −1.76 | 4.22E−03 |
| SCIN | −1.75 | 6.38E−08 |
| FBLN5 | −1.73 | 1.16E−02 |
| ITGAV | −1.73 | 1.17E−04 |
| KIF5C | −1.71 | 5.47E−09 |
| ZNF488 | −1.70 | 1.01E−04 |
| ITIH1 | −1.70 | 1.95E−06 |
| PDZK1P1 | −1.70 | 7.05E−04 |
| SBSN | −1.69 | 1.58E−02 |
| FBRSL1 | −1.68 | 1.87E−04 |
| CHL1 | −1.67 | 3.00E−04 |
| TF | −1.66 | 1.67E−05 |
| COL3A1 | −1.66 | 5.22E−02 |
| MMP1 | −1.63 | 4.10E−04 |
| GRIN2C | −1.62 | 2.48E−02 |
| CAMP | −1.61 | 4.54E−05 |
| BLMH | −1.58 | 5.24E−04 |
| ADA | −1.55 | 2.61E−06 |
| ALB | −1.54 | 3.86E−07 |
| TIMP3 | −1.53 | 1.81E−03 |
| HK1 | −1.49 | 9.51E−07 |
| LCN1 | −1.47 | 4.32E−03 |
| TGM1 | −1.44 | 4.86E−02 |
| COMP | −1.44 | 1.07E−05 |
| SLC26A11 | −1.40 | 2.30E−03 |
| IGLV3-9 | −1.39 | 8.77E−08 |
| IGLV3-21 | −1.39 | 4.11E−07 |
| VPS13A | −1.37 | 2.81E−05 |
| IGHV1-69 | −1.35 | 4.84E−05 |
| PRXL2B | −1.34 | 2.79E−02 |
| IGHA2 | −1.33 | 3.04E−03 |
| CPQ | −1.33 | 1.19E−08 |
| PAICS | −1.31 | 2.83E−04 |
| ABCC4 | −1.28 | 5.62E−07 |
| IGHV3-74 | −1.28 | 6.09E−05 |
| IGKV1D-16 | −1.26 | 2.09E−03 |
| DNAH11 | −1.26 | 1.07E−04 |
| IGKV1D-39 | −1.25 | 2.39E−02 |
| ZGRF1 | −1.24 | 1.53E−06 |
| TGFB1 | −1.21 | 1.31E−04 |
| DCD | −1.20 | 1.97E−04 |
| KRT9 | −1.20 | 4.97E−09 |
| IGHV4-4 | −1.20 | 9.57E−03 |
| XPNPEP2 | −1.19 | 3.50E−03 |
| PKP1 | −1.16 | 9.92E−05 |
| RASGRP2 | −1.16 | 8.93E−04 |
| CLEC3B | −1.15 | 9.25E−04 |
| LRP1B | −1.14 | 3.59E−02 |
| IGKV3D-15 | −1.13 | 2.04E−05 |
| ATAD2 | −1.13 | 2.38E−03 |
| IGHV5-10-1 | −1.12 | 6.47E−05 |
| TPM4 | −1.11 | 3.11E−06 |
| KRT2 | −1.10 | 9.67E−08 |
| IGHD | −1.10 | 8.57E−06 |
| IGHV3-43 | −1.09 | 2.07E−04 |
| PATJ | −1.09 | 9.49E−03 |
| ZNF425 | −1.08 | 5.60E−02 |
| IGHV1OR15-1 | −1.08 | 3.68E−03 |
| CCDC180 | −1.04 | 1.48E−04 |
| EIF4A1 | −1.03 | 3.56E−04 |
| IGLV3-25 | −1.03 | 1.36E−06 |
| F13B | −1.02 | 2.69E−02 |
| MSN | −1.01 | 3.22E−06 |
| CSTA | −1.01 | 6.82E−07 |
| FAM47E-STBD1 | −1.00 | 4.16E−05 |

### Example 14. smRNAseq Profiling for HMC Cells Vs HMC-EVs

HMCs were generated from the same bank of frozen hemangioblasts described in Example 1. HMCs were generated and passaged up to six passages (P6) according to the method described in Example 1. Extracellular vesicles (EVs) were purified from HMCs (HMC-EVs) by tangential flow filtration (TFF). smRNAseq profiling was performed for HMC-EVs (n=3) and HMCs (n=3).

Table 21 shows smRNAs that were more highly abundant in the HMC-EVs compared with HMCs. Table 22 shows smRNAs that were more highly abundant in the HMCs compared with HMC-EVs.

TABLE 21
miRNAs with higher levels in HMC-EVs compared to HMCs

| miRNA ID | Fold difference | P value |
|---|---|---|
| hsa-miR-1290 | −3237.80 | 0 |
| hsa-miR-122-5p | −2697.49 | 6.83E−40 |
| hsa-miR-223-3p | −1451.47 | 1.70E−20 |
| hsa-miR-338-5p | −1191.28 | 1.44E−19 |
| hsa-miR-451a | −672.00 | 3.99E−26 |
| hsa-miR-320c | −513.86 | 0 |
| hsa-miR-1246 | −485.03 | 0 |
| hsa-miR-320d | −447.23 | 0 |
| hsa-miR-9-3p | −333.60 | 1.98E−12 |
| hsa-miR-139-3p | −282.32 | 4.45E−12 |
| hsa-miR-150-5p | −268.41 | 8.85E−12 |
| hsa-miR-423-5p | −253.84 | 0 |
| hsa-miR-4516 | −241.56 | 4.44E−11 |
| hsa-miR-4433b-5p | −235.62 | 5.88E−11 |
| hsa-miR-223-5p | −222.70 | 9.27E−11 |
| hsa-miR-3138 | −213.62 | 3.87E−17 |
| hsa-miR-4433b-3p | −184.67 | 9.31E−10 |
| hsa-miR-11400 | −170.22 | 1.16E−09 |
| hsa-miR-486-5p | −159.91 | 0 |
| hsa-miR-4738-3p | −135.10 | 7.01E−10 |
| hsa-miR-5010-5p | −130.91 | 1.30E−08 |
| hsa-miR-144-3p | −126.67 | 2.43E−08 |
| hsa-miR-664a-5p | −124.89 | 0 |
| hsa-miR-432-5p | −122.75 | 0 |
| hsa-miR-6809-5p | −117.30 | 1.35E−07 |
| hsa-miR-320b | −110.63 | 0 |
| hsa-miR-4659b-3p | −99.06 | 2.55E−06 |
| hsa-miR-139-5p | −89.21 | 4.42E−08 |
| hsa-miR-142-5p | −82.36 | 0 |
| hsa-miR-320e | −79.30 | 4.88E−14 |
| hsa-miR-363-3p | −76.09 | 2.75E−27 |
| hsa-miR-1273h-5p | −75.36 | 2.36E−06 |
| hsa-miR-3679-5p | −67.01 | 2.62E−24 |
| hsa-miR-584-5p | −63.66 | 0 |
| hsa-miR-2110 | −62.37 | 0 |
| hsa-miR-6877-5p | −59.41 | 5.22E−05 |
| hsa-miR-6862-5p | −58.09 | 4.50E−05 |
| hsa-miR-766-5p | −55.73 | 2.21E−08 |
| hsa-miR-4446-3p | −51.85 | 6.66E−06 |
| hsa-miR-5187-5p | −49.91 | 0.000222 |
| hsa-miR-544b | −47.93 | 0.000163 |
| hsa-miR-320a-3p | −47.66 | 0 |
| hsa-miR-6515-5p | −46.42 | 1.83E−05 |
| hsa-miR-342-5p | −43.34 | 2.57E−20 |
| hsa-miR-338-3p | −43.19 | 0.000301 |
| hsa-miR-3154 | −41.01 | 0.000725 |
| hsa-miR-193b-5p | −40.02 | 0 |
| hsa-miR-628-3p | −39.55 | 0 |
| hsa-miR-4429 | −36.37 | 0.000548 |
| hsa-miR-6837-5p | −36.33 | 7.33E−05 |
| hsa-miR-7849-3p | −35.48 | 0.004653 |
| hsa-miR-122-3p | −34.71 | 0.001147 |
| hsa-miR-6866-5p | −32.18 | 0.001507 |
| hsa-miR-6735-5p | −31.29 | 0.00492 |
| hsa-miR-4743-5p | −30.88 | 0.001291 |
| hsa-miR-3177-3p | −30.77 | 5.76E−21 |
| hsa-miR-7854-3p | −28.89 | 1.52E−05 |
| hsa-miR-6852-5p | −28.85 | 5.35E−12 |
| hsa-miR-126-5p | −28.31 | 0 |
| hsa-miR-1908-5p | −26.94 | 2.76E−17 |
| hsa-miR-323b-3p | −26.80 | 0 |
| hsa-miR-2276-3p | −26.68 | 0.005841 |
| hsa-miR-142-3p | −26.63 | 0.000857 |
| hsa-miR-3175 | −26.50 | 0.002484 |
| hsa-miR-5189-5p | −26.30 | 0.001287 |
| hsa-miR-616-3p | −26.22 | 2.88E−05 |
| hsa-miR-144-5p | −26.09 | 0.000808 |
| hsa-miR-4667-5p | −25.94 | 0.000963 |
| hsa-miR-483-5p | −25.40 | 0 |
| hsa-miR-877-5p | −23.99 | 0 |
| hsa-miR-204-3p | −23.92 | 0.012059 |
| hsa-miR-126-3p | −23.32 | 0 |
| hsa-miR-7856-5p | −23.28 | 0.004825 |
| hsa-miR-1273h-3p | −23.10 | 0.004919 |
| hsa-let-7b-5p | −22.10 | 0 |
| hsa-miR-433-3p | −21.75 | 1.59E−09 |
| hsa-miR-3161 | −20.29 | 0.010391 |
| hsa-miR-146a-5p | −20.17 | 0 |
| hsa-miR-1-3p | −20.10 | 0 |
| hsa-miR-6131 | −19.93 | 0.010871 |
| hsa-miR-1262 | −18.98 | 1.96E−18 |
| hsa-miR-10399-5p | −18.72 | 5.36E−15 |
| hsa-miR-5584-5p | −18.07 | 0.013828 |
| hsa-miR-3126-5p | −17.58 | 7.88E−06 |
| hsa-miR-4804-5p | −17.11 | 1.74E−05 |
| hsa-miR-335-5p | −17.04 | 0 |
| hsa-miR-95-3p | −16.80 | 0.005371 |
| hsa-miR-148a-3p | −16.41 | 0 |
| hsa-miR-23b-5p | −15.34 | 6.38E−39 |
| hsa-miR-10b-3p | −15.11 | 0.002094 |
| hsa-miR-3125 | −14.93 | 0.001865 |
| hsa-miR-3187-3p | −14.48 | 7.00E−05 |
| hsa-miR-760 | −14.46 | 1.14E−07 |
| hsa-miR-942-3p | −14.12 | 1.30E−06 |
| hsa-miR-10526-3p | −13.89 | 0.008759 |
| hsa-miR-548j-3p | −13.31 | 0.014203 |
| hsa-miR-3960 | −13.13 | 0.004868 |
| hsa-miR-5189-3p | −13.00 | 0.011457 |
| hsa-miR-4647 | −12.61 | 0.004701 |
| hsa-miR-3622a-5p | −12.43 | 0.001639 |
| hsa-miR-4662a-5p | −12.21 | 2.37E−08 |
| hsa-miR-1299 | −12.13 | 0.000244 |
| hsa-miR-10a-3p | −10.83 | 5.92E−08 |
| hsa-miR-1270 | −10.52 | 6.89E−38 |
| hsa-let-7c-5p | −10.37 | 0 |
| hsa-miR-3944-5p | −9.06 | 0.00445 |
| hsa-miR-3605-5p | −8.98 | 2.29E−17 |
| hsa-miR-3120-3p | −8.97 | 0.003762 |
| hsa-miR-1180-3p | −8.79 | 7.93E−34 |
| hsa-miR-758-5p | −8.33 | 2.17E−05 |
| hsa-miR-3928-3p | −8.18 | 6.01E−05 |
| hsa-miR-7706 | −8.02 | 2.68E−21 |
| hsa-miR-10399-3p | −7.86 | 0.012167 |
| hsa-miR-182-5p | −7.30 | 0 |
| hsa-miR-485-5p | −7.05 | 1.78E−11 |
| hsa-miR-574-5p | −6.67 | 0 |
| hsa-miR-505-5p | −6.42 | 2.25E−07 |
| hsa-miR-1843 | −6.34 | 3.29E−18 |
| hsa-miR-3934-5p | −6.20 | 7.24E−07 |
| hsa-miR-543 | −6.20 | 6.64E−15 |
| hsa-miR-654-5p | −5.92 | 1.94E−06 |
| hsa-miR-421 | −5.90 | 1.68E−44 |
| hsa-miR-23a-5p | −5.90 | 0.002561 |
| hsa-miR-548e-3p | −5.88 | 1.03E−24 |
| hsa-miR-4645-3p | −5.71 | 0.010916 |
| hsa-miR-25-5p | −5.55 | 1.87E−12 |
| hsa-miR-196b-5p | −5.35 | 0.009427 |
| hsa-miR-3140-3p | −5.18 | 0.010278 |
| hsa-miR-1301-3p | −5.16 | 2.63E−36 |
| hsa-miR-4435 | −5.13 | 0.006987 |
| hsa-miR-889-3p | −5.02 | 0 |
| hsa-miR-744-5p | −5.01 | 0 |
| hsa-miR-148a-5p | −4.74 | 8.33E−05 |
| hsa-miR-486-3p | −4.74 | 0.005596 |
| hsa-miR-125a-3p | −4.61 | 7.80E−30 |
| hsa-miR-323a-3p | −4.60 | 1.49E−25 |
| hsa-miR-1292-5p | −4.44 | 0.000159 |
| hsa-miR-10b-5p | −4.38 | 0 |
| hsa-miR-365b-5p | −4.37 | 0.000148 |
| hsa-miR-193a-5p | −4.35 | 2.27E−29 |
| hsa-miR-10527-5p | −4.35 | 0.002016 |
| hsa-miR-134-5p | −4.20 | 2.31E−25 |
| hsa-miR-423-3p | −4.02 | 2.84E−34 |
| hsa-miR-3129-5p | −4.00 | 8.78E−05 |
| hsa-miR-942-5p | −3.96 | 7.75E−05 |
| hsa-miR-16-2-3p | −3.80 | 1.32E−22 |
| hsa-miR-101-3p | −3.75 | 0 |
| hsa-miR-495-3p | −3.74 | 1.64E−07 |
| hsa-miR-92b-5p | −3.67 | 0.000132 |
| hsa-miR-369-3p | −3.62 | 5.98E−06 |
| hsa-miR-1197 | −3.51 | 0.003072 |
| hsa-miR-382-5p | −3.49 | 2.65E−15 |
| hsa-miR-1285-3p | −3.42 | 7.67E−06 |
| hsa-miR-30a-3p | −3.13 | 3.03E−18 |
| hsa-miR-656-3p | −3.10 | 2.82E−05 |
| hsa-miR-589-5p | −2.99 | 0 |
| hsa-miR-128-3p | −2.99 | 0 |
| hsa-miR-409-3p | −2.95 | 0 |
| hsa-miR-215-5p | −2.83 | 0.000243 |
| hsa-miR-378i | −2.81 | 0.003797 |
| hsa-miR-382-3p | −2.78 | 4.02E−08 |
| hsa-miR-185-5p | −2.52 | 0 |
| hsa-let-7d-5p | −2.50 | 5.51E−43 |
| hsa-let-7e-5p | −2.48 | 0 |
| hsa-miR-576-3p | −2.45 | 9.25E−09 |
| hsa-miR-652-3p | −2.41 | 4.86E−09 |
| hsa-miR-10a-5p | −2.34 | 3.29E−25 |
| hsa-miR-1304-3p | −2.29 | 1.16E−05 |
| hsa-miR-28-3p | −2.25 | 0 |
| hsa-miR-92a-3p | −2.15 | 6.94E−09 |
| hsa-let-7d-3p | −2.15 | 0.000249 |
| hsa-miR-330-3p | −2.07 | 3.20E−11 |
| hsa-miR-629-5p | −1.84 | 0.000424 |
| hsa-miR-424-3p | −1.82 | 5.89E−08 |
| hsa-miR-30e-3p | −1.78 | 2.84E−06 |
| hsa-miR-378a-3p | −1.78 | 7.49E−23 |
| hsa-miR-146b-5p | −1.71 | 2.30E−20 |
| hsa-miR-654-3p | −1.68 | 4.70E−19 |
| hsa-miR-224-5p | −1.64 | 1.42E−13 |
| hsa-miR-106b-3p | −1.59 | 1.10E−08 |

TABLE 22
miRNAs with higher levels in HMC cells compared to HMC-EVs

| miRNA ID | Fold difference | P value |
|---|---|---|
| hsa-miR-5701 | 347.64 | 9.55E−14 |
| hsa-miR-500a-5p | 93.58 | 3.69E−08 |
| hsa-miR-145-5p | 78.86 | 0 |
| hsa-miR-7974 | 76.40 | 2.30E−11 |
| hsa-miR-4521 | 71.20 | 3.38E−07 |
| hsa-miR-137-3p | 55.66 | 1.57E−09 |
| hsa-miR-152-5p | 54.93 | 2.47E−06 |
| hsa-miR-1260a | 46.60 | 0 |
| hsa-miR-483-3p | 44.67 | 1.22E−05 |
| hsa-miR-12135 | 42.22 | 2.94E−05 |
| hsa-miR-548i | 42.02 | 2.14E−05 |
| hsa-miR-140-5p | 41.64 | 1.64E−13 |
| hsa-miR-5100 | 36.65 | 5.81E−05 |
| hsa-miR-190a-5p | 31.23 | 0.000258 |
| hsa-miR-153-3p | 31.17 | 0.000223 |
| hsa-miR-27a-5p | 29.93 | 4.85E−28 |
| hsa-miR-500b-5p | 27.87 | 0.000394 |
| hsa-let-7c-3p | 27.31 | 0.000354 |
| hsa-miR-4286 | 25.72 | 1.00E−34 |
| hsa-miR-374b-3p | 25.68 | 0.000161 |
| hsa-miR-218-5p | 24.79 | 1.43E−13 |
| hsa-miR-331-3p | 19.83 | 9.12E−41 |
| hsa-miR-301b-3p | 19.57 | 0.002357 |
| hsa-miR-188-3p | 19.51 | 0.002865 |
| hsa-miR-18a-5p | 18.74 | 4.22E−10 |
| hsa-miR-874-5p | 18.67 | 0.003373 |
| hsa-miR-105-5p | 18.22 | 0.003366 |
| hsa-miR-31-3p | 17.35 | 3.79E−06 |
| hsa-let-7a-2-3p | 16.87 | 0.001668 |
| hsa-miR-21-3p | 16.43 | 9.09E−13 |
| hsa-miR-210-5p | 16.37 | 0.005401 |
| hsa-miR-2277-5p | 16.24 | 0.002077 |
| hsa-miR-450a-1-3p | 15.87 | 0.007502 |
| hsa-miR-296-5p | 15.50 | 0.008142 |
| hsa-miR-1260b | 14.94 | 0 |
| hsa-miR-193a-3p | 14.50 | 0.009837 |
| hsa-miR-212-3p | 14.40 | 0.010622 |
| hsa-miR-130a-5p | 14.39 | 0.000405 |
| hsa-miR-542-3p | 14.09 | 5.25E−39 |
| hsa-miR-125a-5p | 13.32 | 0 |
| hsa-miR-7-5p | 13.03 | 0 |
| hsa-miR-4497 | 13.02 | 0.015303 |
| hsa-miR-454-3p | 12.59 | 1.99E−08 |
| hsa-miR-21-5p | 12.45 | 0 |
| hsa-miR-570-3p | 11.59 | 0.009332 |
| hsa-miR-424-5p | 11.54 | 0 |
| hsa-miR-132-5p | 11.48 | 4.52E−07 |
| hsa-miR-125b-5p | 11.40 | 0 |
| hsa-miR-7977 | 11.37 | 3.36E−44 |
| hsa-miR-34b-3p | 11.18 | 0.010197 |
| hsa-miR-93-5p | 10.73 | 0 |
| hsa-miR-199a-5p | 10.58 | 0 |
| hsa-miR-197-3p | 10.19 | 3.63E−29 |
| hsa-miR-210-3p | 9.66 | 2.56E−05 |
| hsa-miR-221-5p | 9.28 | 0 |
| hsa-miR-582-5p | 9.09 | 1.82E−10 |
| hsa-miR-99b-5p | 8.87 | 0 |
| hsa-miR-3940-3p | 8.72 | 0.000688 |
| hsa-miR-103a-3p | 8.70 | 0 |
| hsa-miR-34a-5p | 8.49 | 4.14E−33 |
| hsa-miR-143-5p | 8.47 | 3.58E−38 |
| hsa-miR-31-5p | 8.34 | 0 |
| hsa-miR-24-2-5p | 8.30 | 2.00E−22 |
| hsa-miR-452-5p | 8.25 | 6.71E−22 |
| hsa-miR-874-3p | 8.15 | 9.71E−42 |
| hsa-miR-145-3p | 8.03 | 0 |
| hsa-miR-143-3p | 7.80 | 0 |
| hsa-miR-365a-3p | 7.70 | 0 |
| hsa-miR-365b-3p | 7.70 | 0 |
| hsa-miR-3613-5p | 7.52 | 2.97E−06 |
| hsa-miR-33b-3p | 7.50 | 0.001136 |
| hsa-miR-708-5p | 7.45 | 0 |
| hsa-miR-17-3p | 7.22 | 8.22E−05 |
| hsa-miR-1296-5p | 7.12 | 2.14E−05 |
| hsa-miR-27a-3p | 7.11 | 0 |
| hsa-miR-17-5p | 6.83 | 0 |
| hsa-miR-2682-5p | 6.75 | 1.73E−06 |
| hsa-miR-148b-5p | 6.70 | 6.49E−05 |
| hsa-let-7a-3p | 6.38 | 4.34E−42 |
| hsa-miR-576-5p | 6.38 | 0.000384 |
| hsa-miR-181a-3p | 6.36 | 0 |
| hsa-miR-665 | 6.33 | 9.58E−06 |
| hsa-miR-3130-5p | 6.30 | 0.015272 |
| hsa-let-7i-3p | 6.19 | 0.010866 |
| hsa-miR-30e-5p | 5.99 | 0 |
| hsa-miR-30a-5p | 5.99 | 0 |
| hsa-let-7i-5p | 5.79 | 0 |
| hsa-let-7g-5p | 5.79 | 0 |
| hsa-miR-335-3p | 5.60 | 0 |
| hsa-miR-425-5p | 5.56 | 0 |
| hsa-miR-4454 | 5.55 | 6.45E−30 |
| hsa-miR-20a-5p | 5.46 | 0 |
| hsa-miR-34a-3p | 5.45 | 0.010285 |
| hsa-miR-29a-3p | 5.42 | 0 |
| hsa-miR-362-5p | 5.39 | 1.42E−15 |
| hsa-miR-708-3p | 5.37 | 9.33E−20 |
| hsa-miR-342-3p | 5.25 | 0 |
| hsa-miR-193b-3p | 5.19 | 1.77E−23 |
| hsa-miR-301a-5p | 5.12 | 3.39E−05 |
| hsa-miR-15b-5p | 5.08 | 0 |
| hsa-miR-34c-5p | 5.07 | 0 |
| hsa-miR-345-5p | 5.06 | 1.40E−45 |
| hsa-miR-4636 | 4.99 | 0.010845 |
| hsa-miR-374b-5p | 4.80 | 1.90E−33 |
| hsa-miR-12136 | 4.80 | 0.012744 |
| hsa-miR-4326 | 4.71 | 1.35E−05 |
| hsa-miR-374a-3p | 4.69 | 1.22E−39 |
| hsa-miR-29c-5p | 4.54 | 0.01528 |
| hsa-miR-15a-5p | 4.46 | 1.46E−11 |
| hsa-miR-103a-2-5p | 4.43 | 7.59E−06 |
| hsa-miR-450a-5p | 4.42 | 0 |
| hsa-miR-411-5p | 4.31 | 3.39E−35 |
| hsa-miR-3158-3p | 4.22 | 0.000646 |
| hsa-miR-3117-3p | 4.20 | 0.00018 |
| hsa-miR-409-5p | 4.16 | 0 |
| hsa-miR-548w | 4.11 | 0.007376 |
| hsa-miR-532-3p | 4.06 | 1.86E−05 |
| hsa-miR-106a-5p | 4.06 | 0.000408 |
| hsa-miR-374a-5p | 4.03 | 1.22E−19 |
| hsa-miR-9903 | 4.03 | 0.014869 |
| hsa-miR-181b-3p | 3.99 | 1.83E−09 |
| hsa-miR-214-3p | 3.83 | 0 |
| hsa-miR-99a-5p | 3.83 | 0 |
| hsa-miR-671-5p | 3.80 | 5.71E−07 |
| hsa-let-7e-3p | 3.76 | 0.00696 |
| hsa-miR-100-5p | 3.74 | 0 |
| hsa-miR-106b-5p | 3.71 | 1.27E−09 |
| hsa-miR-339-5p | 3.70 | 1.04E−08 |
| hsa-miR-16-5p | 3.69 | 0 |
| hsa-miR-376c-3p | 3.63 | 0.007115 |
| hsa-miR-582-3p | 3.52 | 0.007728 |
| hsa-miR-561-5p | 3.51 | 0.000356 |
| hsa-miR-30b-5p | 3.50 | 0 |
| hsa-miR-500a-3p | 3.24 | 3.86E−32 |
| hsa-miR-381-3p | 3.17 | 9.94E−05 |
| hsa-miR-130b-5p | 3.17 | 0 |
| hsa-miR-130a-3p | 3.09 | 3.31E−07 |
| hsa-let-7f-1-3p | 3.09 | 0.008094 |
| hsa-miR-194-5p | 3.08 | 3.68E−11 |
| hsa-miR-502-3p | 3.08 | 4.76E−14 |
| hsa-miR-32-5p | 3.07 | 0.015461 |
| hsa-miR-5094 | 3.06 | 0.007148 |
| hsa-miR-125b-2-3p | 2.98 | 1.04E−07 |
| hsa-miR-625-3p | 2.95 | 1.48E−11 |
| hsa-miR-379-5p | 2.91 | 1.68E−09 |
| hsa-miR-484 | 2.82 | 0 |
| hsa-miR-138-5p | 2.80 | 1.76E−16 |
| hsa-miR-148b-3p | 2.78 | 0 |
| hsa-miR-27b-3p | 2.75 | 0 |
| hsa-miR-19b-3p | 2.66 | 1.11E−19 |
| hsa-miR-30c-5p | 2.63 | 3.60E−34 |
| hsa-miR-22-3p | 2.63 | 3.16E−33 |
| hsa-miR-221-3p | 2.62 | 0 |
| hsa-miR-183-5p | 2.58 | 0.000322 |
| hsa-miR-214-5p | 2.56 | 3.02E−05 |
| hsa-miR-2355-5p | 2.55 | 0.000159 |
| hsa-miR-29b-3p | 2.48 | 2.65E−05 |
| hsa-miR-149-5p | 2.40 | 2.02E−05 |
| hsa-miR-4677-3p | 2.34 | 6.76E−06 |
| hsa-miR-98-5p | 2.32 | 1.53E−19 |
| hsa-miR-361-3p | 2.30 | 3.19E−06 |
| hsa-miR-181a-2-3p | 2.29 | 2.46E−17 |
| hsa-miR-370-3p | 2.26 | 4.89E−07 |
| hsa-miR-140-3p | 2.19 | 1.90E−25 |
| hsa-miR-574-3p | 2.16 | 2.43E−06 |
| hsa-miR-127-3p | 2.13 | 1.73E−28 |
| hsa-miR-28-5p | 2.11 | 8.76E−18 |
| hsa-miR-181c-3p | 2.03 | 8.80E−09 |
| hsa-miR-24-3p | 2.02 | 1.08E−28 |
| hsa-miR-136-3p | 2.00 | 1.84E−06 |
| hsa-miR-107 | 2.00 | 0.000144 |
| hsa-miR-199b-5p | 1.99 | 7.66E−26 |
| hsa-miR-26b-5p | 1.91 | 1.80E−17 |
| hsa-miR-191-5p | 1.87 | 1.08E−22 |
| hsa-miR-450b-5p | 1.81 | 1.76E−07 |
| hsa-miR-30d-5p | 1.69 | 2.65E−42 |
| hsa-miR-339-3p | 1.66 | 0.008601 |
| hsa-miR-23b-3p | 1.64 | 3.63E−18 |
| hsa-miR-769-5p | 1.51 | 0.000511 |

While the foregoing description and figures represent exemplary embodiments of the present disclosure, it will be understood that various additions, modifications and substitutions may be made therein without departing from the spirit and scope and range of equivalents of the accompanying claims. In particular, it will be clear to those skilled in the art that the presently disclosed subject matter may be embodied in other forms, structures, arrangements, and with other elements, materials, and components, without departing from the spirit or essential characteristics thereof. In addition, numerous variations in the methods/processes described herein may be made within the scope of the present disclosure without departing from the principles described herein. The presently disclosed embodiments are therefore to be considered in all respects as illustrative and not restrictive. The appended claims should be construed broadly, to include other variants and embodiments of the disclosure, which may be made by those skilled in the art without departing from the scope and range of equivalents.

1. A method of treating a brain injury in a subject suffering from, or suspected of suffering from, a brain injury, the method comprising administering to the subject an effective amount of extracellular vesicles (EVs) secreted from mesenchymal stem cells (HMCs) obtained by in vitro differentiation of pluripotent stem cells, thereby treating the brain injury in the subject.

2. The method of claim 1, wherein the brain injury is selected from the group consisting of stroke, optic neuropathy, traumatic brain injury, cerebral palsy, acquired brain injury, anoxic brain injury, diffuse axonal brain injury, focal brain injury, subdural hematoma, brain aneurysm, and coma.

3. The method of claim 2, wherein the brain injury is stroke.

4. The method of any one of claims 1-3, wherein the method comprises increasing oligodendrocyte and precursor cells in the brain following administration of the EVs secreted from the HMCs (HMC-EVs) into the subject.

5. The method of any one of claims 1-3, wherein the method comprises preserving myelin in the brain following administration of the HMC-EVs into the subject.

6. The method of any one of claims 1-3, wherein the method comprises preventing oxidative damage in neurons following administration of the HMC-EVs into the subject.

7. The method of any one of claims 1-3, wherein the method comprises preventing neuronal death due to glutamate excitotoxicity injury following administration of the HMC-EVs into the subject.

8. The method of any one of claims 1-3, wherein the method comprises reducing tissue loss in the brain following administration of the HMC-EVs into the subject.

9. The method of any one of claims 1-3, wherein the method comprises reducing cell death in the brain following administration of the HMC-EVs into the subject.

10. The method of any one of claims 1-3, wherein the method comprises stimulating pathways involved in the development of neuronal lineage following administration of the HMC-EVs into the subject.

11. The method of any one of claims 1-10, wherein the HMC-EVs are administered systemically.

12. The method of any one of claims 1-10, wherein the HMC-EVs are administered intracerebrally.

13. The method of any one of claims 1-10, wherein the HMC-EVs are administered intrathecally.

14. The method of any one of claims 1-10, wherein the HMC-EVs are administered intracisternally.

15. The method of any one of claims 1-10, wherein the HMC-EVs are administered intraperitoneally.

16. The method of any one of claims 1-15, wherein the subject is a human.

17. The method of any one of claims 1-16, wherein the HMCs are obtained by in vitro differentiation of human pluripotent stem cells.

18. The method of any one of claims 1-17, wherein the pluripotent stem cells are further differentiated into hemangioblasts.

19. The method of any one of claims 1-18, wherein the pluripotent stem cells are embryonic stem cells.

20. The method of any one of claims 1-18, wherein the pluripotent stem cells are induced pluripotent stem cells.

21. The method of claim 20, wherein the induced pluripotent stem cells are produced by contacting a cell with one or more reprogramming factors.

22. The method of any one of claims 1-21, wherein the HMC-EVs express at least one of the miRNA in Table 9 at a higher level compared to EVs secreted from umbilical cord blood-derived mesenchymal stem cells (UCB-MSC-EVs).

23. The method of any one of claims 1-22, wherein the HMC-EVs express at least one of the miRNA in Table 10 at a lower level compared to UCB-MSC-EVs.

24. The method of any one of claims 1-23, wherein the HMC-EVs express at least one of the miRNA in Table 11 at a higher level compared to EVs secreted from bone marrow-derived mesenchymal stem cells (BM-MSC-EVs).

25. The method of any one of claims 1-24, wherein the HMC-EVs express at least one of the miRNA in Table 12 at a lower level compared to BM-MSC-EVs.

26. The method of any one of claims 1-25, wherein the HMC-EVs express at least one of the miRNA in Table 13 at a higher level compared to EVs secreted from adipose tissue-derived mesenchymal stem cells (AD-MSC-EVs).

27. The method of any one of claims 1-26, wherein the HMC-EVs express at least one of the miRNA in Table 14 at a lower level compared to AD-MSC-EVs.

28. The method of any one of claims 1-27, wherein the HMC-EVs express at least one of the proteins in Table 15 at a higher level compared to UCB-MSC-EVs.

29. The method of any one of claims 1-28, wherein the HMC-EVs express at least one of the proteins in Table 16 at a lower level compared to UCB-MSC-EVs.

30. The method of any one of claims 1-29, wherein the HMC-EVs express at least one of the proteins in Table 17 at a higher level compared to BM-MSC-EVs.

31. The method of any one of claims 1-30, wherein the HMC-EVs express at least one of the proteins in Table 18 at a lower level compared to BM-MSC-EVs.

32. The method of any one of claims 1-31, wherein the HMC-EVs express at least one of the proteins in Table 19 at a higher level compared to AD-MSC-EVs.

33. The method of any one of claims 1-32, wherein the HMC-EVs express at least one of the proteins in Table 20 at a lower level compared to AD-MSC-EVs.

34. The method of any one of claims 1-33, wherein the HMC-EVs express at least one of the miRNA in Table 21 at a higher level compared to the HMCs.

35. The method of any one of claims 1-34, wherein the HMC-EVs express at least one of the miRNA in Table 22 at a lower level compared to the HMCs.

36. The method of any one of claims 1-35, wherein the HMC-EVs express at least one of the miRNAs selected from the group consisting of hsa-miR-125b-5p, hsa-miR-181a-5p, hsa-miR-199b-5p, hsa-miR-21-5p, hsa-miR-23a-3p, hsa-miR-125a-5p, hsa-miR-106a-5p+hsa-miR-17-5p and hsa-miR-221-3p at a higher level compared to BM-MSC-EVs, UCB-MSC-EVs, and/or AD-MSC-EVs.

37. The method of any one of claims 1-36, wherein the HMC-EVs express at least one of the proteins selected from the group consisting of ALDOC, ANXA5, APBB2, BASP1, CAV1, CD81, CD99, CKM, EPB41L3, FDPS, GNAQ, GNG12, GP9, H2AC20, H2AC21, H3-3A, H3-7, H4-16, HLA-A, ITGA2, KPNA2, KRAS, KRT4, LRRC59, MAMDC2, MARCKSL1, MDGA1, MERTK, MFGE8, MMP14, MVP, PCDH1, PDGFRB, PDIA3, RPL13, RPS18, RPS3A, RPS4X, SDCBP, SLC2A1, SLC3A2, TAGLN2, TNC, TSPAN14, TSPAN33, TSPAN9, TTYH3, UCHL1, VAT1, YWHAB, and YWHAQ at a higher level compared to BM-MSC-EVs, UCB-MSC-EVs, and/or AD-MSC-EVs.

38. The method of any one of claims 1-37, wherein the HMC-EVs express at least one of the proteins selected from the group consisting of ADGRG6, AGRN, ANXA6, APOC4, ARHGAP1, ARGHDIA, ARL8A, ARPC5, B2M, BBS1, BLVRA, BST1, CA2, CCN2, CCNB3, CD34, CD36, CD47, CORO1A, DTD1, EEF1D, EEF1G, ENG, ESD, GNAI2, GNB1, H1-3, H2BC15, HIP1, KIF11, LAMP1, LAP3, LGALS1, LTBP3, MAPK3, MARCKS, MBTD1, MDH1, MOB1B, MYL12B, MYO1F, MYO3A, NIBAN2, PEBP1, PF4, PGAP1, PLOD1, PPP2R1A, PRSS23, PXDN, RALA, RAP2A, RPS13, RPS3, RPSA, S100A 11, SLC44A1, SLC44A2, SLTM, SMG1, SPARC, SRSF8, STRADB, STX11, STXBP2, TGM2, TPP1, TPTE2, TRIM5, TRPM2, TUBA8, TUBB3, VCAN, YWHAE, and ZFN607 at a higher level compared to BM-MSC-EVs, UCB-MSC-EVs, and/or AD-MSC-EVs.

39. The method of any one of claims 1-38, wherein the HMC-EVs express at least one of the proteins selected from the group consisting of ADIPOQ, CAT, CEP290, IGLV6-57, TAS2R33, and TMEM198 at a lower level compared to BM-MSC-EVs, UCB-MSC-EVs, and/or AD-MSC-EVs.

40. The method of any one of claims 1-39, wherein the HMC-EVs express at least one of the proteins selected from the group consisting of AKAP9, ALB, ALOX5, APLP2, CD109, CDSN, CHST9, ERC1, F11, ARMCX5, LAMB4, LRRTM2, LTF, MSH6, OAF, OLFML3, PAK6, RGS14, SEMA7A, SURF1, and TRIM4 at a lower level compared to BM-MSC-EVs, UCB-MSC-EVs, and/or AD-MSC-EVs.

41. The method of any one of claims 1-40, wherein about 1×10⁶ to about 1×10³ HMC-EVs are administered to the subject.

42. The method of any one of claims 1-41, wherein about 10×10¹⁰ or about 30×10¹⁰ HMC-EVs are administered to the subject.

43. The method of any one of claims 1-42, wherein the HMC-EVs are administered in a pharmaceutical composition.

44. The method of claim 43, wherein the pharmaceutical composition comprises
(a) a buffer, maintaining the solution at a physiological pH;
(b) at least 2 mM or at least 0.05% (w/v) glucose; and
(c) an osmotically active agent maintaining the solution at a physiological osmolarity.

45. The method of claim 44, wherein the glucose is D-glucose (Dextrose).

46. The method of claim 44, wherein the osmotically active agent is a salt.

47. The method of claim 46, wherein the salt is sodium chloride.

48. The method of any one of claims 1-47, further comprising administering to the subject an effective amount of HMCs obtained by in vitro differentiation of pluripotent stem cells.

49. A method of treating a brain injury in a subject suffering from, or suspected of suffering from, a brain injury, the method comprising administering to the subject an effective amount of mesenchymal stem cells (HMCs) obtained by in vitro differentiation of pluripotent stem cells, thereby treating the brain injury in the subject.

50. The method of claim 49, wherein the brain injury is selected from the group consisting of stroke, optic neuropathy, traumatic brain injury, cerebral palsy, acquired brain injury, anoxic brain injury, diffuse axonal brain injury, focal brain injury, subdural hematoma, brain aneurysm, and coma.

51. The method of claim 50, wherein the brain injury is stroke.

52. The method of any one of claims 49-51, wherein the method comprises preserving myelin in the brain following administration of the HMCs into the subject.

53. The method of any one of claims 49-51, wherein the method comprises suppressing neuroinflammatory responses following administration of the HMCs into the subject.

54. The method of any one of claims 49-51, wherein the method comprises reducing microglial and astrocyte activation in the brain following administration of the HMCs into the subject.

55. The method of any one of claims 49-51, wherein the method comprises stimulating pathways involved in cell survival following administration of the HMCs into the subject.

56. The method of any one of claims 49-51, wherein the method comprises stimulating expression of a neuroprotective gene in the brain following administration of the HMCs into the subject.

57. The method of claim 56, wherein the neuroprotective gene is selected from the group consisting of heat shock protein family B member 1 (HSPB1), insulin-like growth factor 1 (IGF2), and secreted phosphoprotein 1 (SPP1).

58. The method of any one of claims 49-51, wherein the method comprises stimulating pathways involved in synaptic transmission in the brain following administration of the HMCs into the subject.

59. The method of any one of claims 49-51, wherein the method comprises stimulating pathways involved in the development of neuronal lineage following administration of the HMCs into the subject.

60. The method of any one of claims 49-51, wherein the method comprises reducing apoptosis following administration of the HMCs into the subject.

61. The method of claim 50, wherein the brain injury is traumatic brain injury.

62. The method of claim 61, wherein the method comprises reducing tissue loss in the brain following administration of the HMCs into the subject.

63. The method of claim 61 or 62, wherein the method comprises reducing cell death in the brain following administration of the HMCs into the subject.

64. The method of any one of claims 61-63, wherein the method comprises increasing neurogenesis following the administration of the HMCs into the subject.

65. The method of any one of claims 61-64, wherein the method comprises reducing the presence of microglia and macrophages in the cortex and striatum following the administration of the HMCs into the subject.

66. The method of any one of claims 61-65, wherein the method comprises reducing inflammation of the spleen following the administration of the HMCs into the subject.

67. The method of any one of claims 61-66, wherein the method comprises migration of HMCs across the blood-brain barrier to the cortex, striatum, and/or hippocampus.

68. The method of claim 50, wherein the brain injury is cerebral palsy.

69. The method of claim 68, wherein the method comprises reducing apoptosis in the brain following administration of the HMCs into the subject.

70. The method of claim 68 or 69, wherein the method comprises reducing lesion size in the brain following administration of the HMCs into the subject.

71. The method of any one of claims 68-70, wherein the method comprises reducing microglial and astrocyte activation in the brain following administration of the HMCs into the subject.

72. The method of any one of claims 68-71, wherein the method comprises preserving myelin of the corpus callosum following administration of the HMCs into the subject.

73. The method of any one of claims 68-72, wherein the method comprises at least a partial rescue of Olig2 in the brain following administration of the HMCs into the subject.

74. The method of any one of claims 49-73, wherein the HMCs are administered systemically.

75. The method of any one of claims 49-73, wherein the HMCs are administered intracerebrally.

76. The method of any one of claims 49-73, wherein the HMCs are administered intrathecally.

77. The method of any one of claims 49-73, wherein the HMCs are administered intracisternally.

78. The method of any one of claims 49-73, wherein the HMCs are administered intraperitoneally.

79. The method of any one of claims 49-78, wherein the mesenchymal stem cells are human cells.

80. The method of any one of claims 49-79, wherein the subject is a human.

81. The method of any one of claims 49-80, wherein the pluripotent stem cells are further differentiated into hemangioblasts.

82. The method of any one of claims 49-81, wherein the pluripotent stem cells are embryonic stem cells.

83. The method of any one of claims 49-82, wherein the pluripotent stem cells are induced pluripotent stem cells.

84. The method of any one of claims 49-83, wherein the pluripotent stem cells are human pluripotent stem cells.

85. The method of any one of claims 49-84, wherein the HMCs have been passaged no more than 5 times in vitro before administration into the subject.

86. The method of any one of claims 49-85, wherein the HMCs express at least one of the genes in Table 3 at a higher level compared to bone marrow-derived MSCs (BM-MSCs).

87. The method of any one of claims 49-85, wherein the HMCs express at least one of the genes in Table 4 at a lower level compared to BM-MSCs.

88. The method of any one of claims 49-85, wherein the HMCs express at least one of the genes in Table 5 at a higher level compared to umbilical cord blood-derived MSCs (UCB-MSCs).

89. The method of any one of claims 49-85, wherein the HMCs express at least one of the genes in Table 6 at a lower level compared to UCB-MSCs.

90. The method of any one of claims 49-85, wherein the HMCs express at least one of the genes in Table 7 at a higher level compared to adipose tissue-derived MSCs (AD-MSCs).

91. The method of any one of claims 49-85, wherein the HMCs express at least one of the genes in Table 8 at a lower level compared to AD-MSCs.

92. The method of any one of claims 49-85, wherein the HMCs express, in a basal state, mRNA encoding interleukin-6 (IL-6) at a level less than ten percent of the IL-6 mRNA level expressed by BM-MSCs in a basal state and wherein the HMCs express, in a basal state, mRNA encoding CD24 at a level that is greater than the CD24 mRNA level expressed by BM-MSCs in a basal state.

93. The method of any one of claims 49-85, wherein the HMCs express at least one of the genes selected from the group consisting of CALR, UBB, PKM, CXCL8, C15orf48, PSME2, TPM3, ANKRD1, PFN1, SRGN, ACTB, MDK, TAGLN2, CFL1, HSP90AA1, HSPA8, CXCL12, UCHL1, HMGA2, HMGA1, HN1, PTMA, SP90AB1, PRDX1, GSTP1, KRT18, IGFBP4, CALD1, COL4A1, COL4A2, and GAPDH at a higher level compared to AD-MSCs.

94. The method of any one of claims 49-85, wherein the HMCs express at least one of the genes selected from the group consisting of TMSB4X, ACTG1, GSTP1, KRT18, IGFBP5, NPY, KRT8, PRDX6, MDK, DKK3, UCHL1, TUBB3, HN1, PTMA, HSP90AB1, HMGA1, HSPA8, TAGLN2, ANKRD1, PFN1, CYBA, and UBB at a higher level compared to AD-MSCs.

95. The method of any one of claims 49-85, wherein the HMCs express at least one of the genes selected from the group consisting of SERPINE1, ACTA2, TPM2, CTGF, SERPINE2, CRYAB, ELN, MFGE8, ANXA2, POSTN, VIM, MFAP5, ISLR, THBS1, TIMP3, DKK1, COL6A3, COL6A1, TPT1, BCYRN1, COL1A1, SPARC, TPM1, BGN, COL1A2, COL3A1, TGFBI, CRLF1, COMP, NEAT1, MT-CO3, MT-CO2, MT-ATP8, MT-CYB, MT-CO1, MT-ATP6, MT-ND4, MT-ND4L, MT-ND5, MT-ND6, MT-ND3, MT-ND1, MT-ND2, GREM1, TMSB4X, ITGB1, LMNA, H2AFZ, FTL, EEF1G, NPM1, EEF1A1, RACK1, ACTG1, and TPM4 at a lower level compared to AD-MSCs.

96. The method of any one of claims 49-85, wherein the HMCs express at least one of the genes selected from the group consisting of SERPINE1, S100A6, CD59, POSTN, VIM, MFAP5, ISLR, THBS1, COL6A3, TIMP3, ELN, ANXA2, COL1A1, BCYRN1, CCDC80, COL6A1, COL6A2, BGN, COL1A2, COL3A1, TGFB1, CRLF1, COMP, and GREM1 at a lower level compared to AD-MSCs.

97. The method of any one of claims 49-85, wherein the HMCs express at least one of the genes selected from the group consisting of MT1X, MT1G, TMSB10, CCL8, INHBA, CTSB, SERPINB2, ADM, APOL1, FTH1, CCL2, CCL5, CSF1, IL1B, IGFBP3, P4HB, DCN, FSTL1, ANXA5, LOX, CD63, CTSZ, FN1, LGALS1, LDHA, RCN3, MMP2, and TIMP1 at a lower level compared to AD-MSCs.

98. The method of any one of claims 49-85, wherein the HMCs express at least one of the genes selected from the group consisting of PPIA, NPM1, HNRNPA1, IGFBP5, KRT19, KRT18, GSTP1, TUBB, TUBA IB, KRT8, HN1, PTMA, TUBA1C, HSPA8, HMGA1, CFL1, MYL6, ACTB, UCHL1, TAGLN2, MDK, GREM1, MMP1, and CTSC at a higher level compared to BM-MSCs.

99. The method of any one of claims 49-85, wherein the HMCs express at least one of the genes selected from the group consisting of ANXA2, TPT1, VIM, COL6A1, BGN, COL6A2, CTGF, TIMP3, ACTA2, COL3A1, SPARC, ITGB1, SERPINH1, TPM2, TGFBI, COL1A1, TPM1, COL6A3, TPM4, SERPINE2, CALD1, COL1A2, TAGLN, MYL9, MT-RNR2, POSTN at a lower level compared to BM-MSCs.

100. The method of any one of claims 49-85, wherein the HMCs express at least one of the miRNA in Table 21 at a lower level compared to the HMC-EVs.

101. The method of any one of claims 49-85, wherein the HMCs express at least one of the miRNA in Table 22 at a higher level compared to the HMC-EVs.

102. The method of any one of claims 49-101, wherein about 1×10⁶ to about 1×10¹³ HMCs are administered to the subject.

103. The method of any one of claims 49-102, wherein the HMCs are administered in a pharmaceutical composition.

104. The method of claim 103, wherein the pharmaceutical composition comprises
(a) a buffer, maintaining the solution at a physiological pH;
(b) at least 2 mM or at least 0.05% (w/v) glucose; and
(c) an osmotically active agent, maintaining the solution at a physiological osmolarity.

105. The method of claim 104, wherein the glucose is D-glucose (Dextrose).

106. The method of claim 104, wherein the osmotically active agent is a salt.

107. The method of claim 106, wherein the salt is sodium chloride.

108. A composition comprising HMCs obtained by in vitro differentiation of pluripotent stem cells, wherein the HMCs express at least one of the genes selected from the group consisting of CALR, UBB, PKM, CXCL8, C15orf48, PSME2, TPM3, ANKRD1, PFN1, SRGN, ACTB, MDK, TAGLN2, CFL1, HSP90AA1, HSPA8, CXCL12, UCHL1, HMGA2, HMGA1, HN1, PTMA, SP90AB1, PRDX1, GSTP1, KRT18, IGFBP4, CALD1, COL4A1, COL4A2, and GAPDH at a higher level compared to AD-MSCs.

109. A composition comprising HMCs obtained by in vitro differentiation of pluripotent stem cells, wherein the HMCs express at least one of the genes selected from the group consisting of TMSB4X, ACTG1, GSTP1, KRT18, IGFBP5, NPY, KRT8, PRDX6, MDK, DKK3, UCHL1, TUBB3, HN1, PTMA, HSP90AB1, HMGA1, HSPA8, TAGLN2, ANKRD1, PFN1, CYBA, and UBB at a higher level compared to AD-MSCs.

110. A composition comprising HMCs obtained by in vitro differentiation of pluripotent stem cells, wherein the HMCs express at least one of the genes selected from the group consisting of PPIA, NPM1, HNRNPA1, IGFBP5, KRT19, KRT18, GSTP1, TUBB, TUBA1B, KRT8, HN1, PTMA, TUBA1C, HSPA8, HMGA1, CFL1, MYL6, ACTB, UCHL1, TAGLN2, MDK, GREM1, MMP1, and CTSC at a higher level compared to BM-MSCs.

111. A composition comprising HMCs obtained by in vitro differentiation of pluripotent stem cells, wherein the HMCs express at least one of the genes selected from the group consisting of SERPINE1, ACTA2, TPM2, CTGF, SERPINE2, CRYAB, ELN, MFGE8, ANXA2, POSTN, VIM, MFAP5, ISLR, THBS1, TIMP3, DKK1, COL6A3, COL6A1, TPT1, BCYRN1, COL1A1, SPARC, TPM1, BGN, COL1A2, COL3A1, TGFBI, CRLF1, COMP, NEAT1, MT-CO3, MT-CO2, MT-ATP8, MT-CYB, MT-CO1, MT-ATP6, MT-ND4, MT-ND4L, MT-ND5, MT-ND6, MT-ND3, MT-ND1, MT-ND2, GREM1, TMSB4X, ITGB1, LMNA, H2AFZ, FTL, EEF1G, NPM1, EEF1A1, RACK1, ACTG1, and TPM4 at a lower level compared to AD-MSCs.

112. A composition comprising HMCs obtained by in vitro differentiation of pluripotent stem cells, wherein the HMCs express at least one of the genes selected from the group consisting of SERPINE1, S100A6, CD59, POSTN, VIM, MFAP5, ISLR, THBS1, COL6A3, TIMP3, ELN, ANXA2, COL1A1, BCYRN1, CCDC80, COL6A1, COL6A2, BGN, COL1A2, COL3A1, TGFB1, CRLF1, COMP, and GREM1 at a lower level compared to AD-MSCs.

113. A composition comprising HMCs obtained by in vitro differentiation of pluripotent stem cells, wherein the HMCs express at least one of the genes selected from the group consisting of MT1X, MT1G, TMSB10, CCL8, INHBA, CTSB, SERPINB2, ADM, APOL1, FTH1, CCL2, CCL5, CSF1, IL1B, IGFBP3, P4HB, DCN, FSTL1, ANXA5, LOX, CD63, CTSZ, FN1, LGALS1, LDHA, RCN3, MMP2, and TIMP1 at a lower level compared to AD-MSCs.

114. A composition comprising HMCs obtained by in vitro differentiation of pluripotent stem cells, wherein the HMCs express at least one of the genes selected from the group consisting of ANXA2, TPT1, VIM, COL6A1, BGN, COL6A2, CTGF, TIMP3, ACTA2, COL3A1, SPARC, ITGB1, SERPINH1, TPM2, TGFBI, COL1A1, TPM1, COL6A3, TPM4, SERPINE2, CALD1, COL1A2, TAGLN, MYL9, MT-RNR2, POSTN at a lower level compared to BM-MSCs.

115. The composition of any one of claims 108-114, wherein the HMCs further express at least one of the genes in Table 3 at a higher level compared to BM-MSCs.

116. The composition of any one of claims 108-114, wherein the HMCs further express at least one of the genes in Table 4 at a lower level compared to BM-MSCs.

117. The composition of any one of claims 108-114, wherein the HMCs further express at least one of the genes in Table 5 at a higher level compared to UCB-MSCs.

118. The composition of any one of claims 108-114, wherein the HMCs further express at least one of the genes in Table 6 at a lower level compared to UCB-MSCs.

119. The composition of any one of claims 108-114, wherein the HMCs further express at least one of the genes in Table 7 at a higher level compared to AD-MSCs.

120. The composition of any one of claims 108-114, wherein the HMCs further express at least one of the genes in Table 8 at a lower level compared to AD-MSCs.

121. A pharmaceutical composition comprising the HMCs of any one of claims 108-114, and a pharmaceutically acceptable carrier.

122. A population of HMC-EVs of any one of claims 108-114.

123. The population of EVs of claim 122, wherein the HMC-EVs express at least one of the miRNA in Table 9 at a higher level compared UCB-MSC-EVs.

124. The population of EVs of claim 122 or 123, wherein the HMC-EVs express at least one of the miRNA in Table 10 at a lower level compared to UCB-MSC-EVs.

125. The population of EVs of any one of claims 122-124, wherein the HMC-EVs express at least one of the miRNA in Table 11 at a higher level compared to BM-MSC-EVs.

126. The population of EVs of any one of claims 122-125, wherein the HMC-EVs express at least one of the miRNA in Table 12 at a lower level compared to BM-MSC-EVs.

127. The population of EVs of any one of claims 122-126, wherein the HMC-EVs express at least one of the miRNA in Table 13 at a higher level compared to AD-MSC-EVs.

128. The population of EVs of any one of claims 122-127, wherein the HMC-EVs express at least one of the miRNA in Table 14 at a lower level compared to AD-MSC-EVs.

129. The population of EVs of any one of claims 122-128, wherein the HMC-EVs express at least one of the proteins in Table 15 at a higher level compared to UCB-MSC-EVs.

130. The population of EVs of any one of claims 122-129, wherein the HMC-EVs express at least one of the proteins in Table 16 at a lower level compared to UCB-MSC-EVs.

131. The population of EVs of any one of claims 122-130, wherein the HMC-EVs express at least one of the proteins in Table 17 at a higher level compared to BM-MSC-EVs.

132. The population of EVs of any one of claims 122-131, wherein the HMC-EVs express at least one of the proteins in Table 18 at a lower level compared to BM-MSC-EVs.

133. The population of EVs of any one of claims 122-132, wherein the HMC-EVs express at least one of the proteins in Table 19 at a higher level compared to AD-MSC-EVs.

134. The population of EVs of any one of claims 122-133, wherein the HMC-EVs express at least one of the proteins in Table 20 at a lower level compared to AD-MSC-EVs.

135. The population of EVs of any one of claims 122-134, wherein the HMC-EVs express at least one of the miRNA in Table 21 at a higher level compared to the HMCs.

136. The population of EVs of any one of claims 122-135, wherein the HMC-EVs express at least one of the miRNA in Table 22 at a lower level compared to the HMCs.

137. The population of EVs of any one of claims 122-136, wherein the HMC-EVs express at least one of the miRNAs selected from the group consisting of hsa-miR-125b-5p, hsa-miR-181a-5p, hsa-miR-199b-5p, hsa-miR-21-5p, hsa-miR-23a-3p, hsa-miR-125a-5p, hsa-miR-106a-5p+hsa-miR-17-5p and hsa-miR-221-3p at a higher level compared to BM-MSC-EVs, UCB-MSC-EVs, and/or AD-MSC-EVs.

138. The population of EVs of any one of claims 122-137, wherein the HMC-EVs express at least one of the proteins selected from the group consisting of ALDOC, ANXA5, APBB2, BASP1, CAV1, CD81, CD99, CKM, EPB41L3, FDPS, GNAQ, GNG12, GP9, H2AC20, H2AC21, H3-3A, H3-7, H4-16, HLA-A, ITGA2, KPNA2, KRAS, KRT4, LRRC59, MAMDC2, MARCKSL1, MDGA1, MERTK, MFGE8, MMP14, MVP, PCDH1, PDGFRB, PDIA3, RPL13, RPS18, RPS3A, RPS4X, SDCBP, SLC2A1, SLC3A2, TAGLN2, TNC, TSPAN14, TSPAN33, TSPAN9, TTYH3, UCHL1, VAT1, YWHAB, and YWHAQ at a higher level compared to BM-MSC-EVs, UCB-MSC-EVs, and/or AD-MSC-EVs.

139. The population of EVs of any one of claims 122-138, wherein the HMC-EVs express at least one of the proteins selected from the group consisting of ADGRG6, AGRN, ANXA6, APOC4, ARHGAP1, ARGHDIA, ARL8A, ARPC5, B2M, BBS1, BLVRA, BST1, CA2, CCN2, CCNB3, CD34, CD36, CD47, CORO1A, DTD1, EEF1D, EEF1G, ENG, ESD, GNAI2, GNB1, H1-3, H2BC15, HIP1, KIF11, LAMP1, LAP3, LGALS1, LTBP3, MAPK3, MARCKS, MBTD1, MDH1, MOB1B, MYL12B, MYO1F, MYO3A, NIBAN2, PEBP1, PF4, PGAP1, PLOD1, PPP2R1A, PRSS23, PXDN, RALA, RAP2A, RPS13, RPS3, RPSA, S100A 11, SLC44A 1, SLC44A2, SLTM, SMG1, SPARC, SRSF8, STRADB, STX11, STXBP2, TGM2, TPP1, TPTE2, TRIM5, TRPM2, TUBA8, TUBB3, VCAN, YWHAE, and ZFN607 at a higher level compared to BM-MSC-EVs, UCB-MSC-EVs, and/or AD-MSC-EVs.

140. The population of HMC-EVs of any one of claims 122-139, wherein the HMC-EVs express at least one of the proteins selected from the group consisting of ADIPOQ, CAT, CEP290, IGLV6-57, TAS2R33, and TMEM198 at a lower level compared to BM-MSC-EVs, UCB-MSC-EVs, and/or AD-MSC-EVs.

141. The population of HMC-EVs of any one of claims 122-140, wherein the HMC-EVs express at least one of the proteins selected from the group consisting of AKAP9, ALB, ALOX5, APLP2, CD109, CDSN, CHST9, ERC1, F11, ARMCX5, LAMB4, LRRTM2, LTF, MSH6, OAF, OLFML3, PAK6, RGS14, SEMA7A, SURF1, and TRIM4 at a lower level compared to BM-MSC-EVs, UCB-MSC-EVs, and/or AD-MSC-EVs.

142. A pharmaceutical composition comprising the HMC-EVs of any one of claims 122-141, and a pharmaceutically acceptable carrier.

143. A population of HMC-EVs obtained by in vitro differentiation of pluripotent stem cells, wherein the HMC-EVs express at least one of the miRNAs in Table 9 at a higher level compared to UCB-MSC-EVs.

144. A population of HMC-EVs obtained by in vitro differentiation of pluripotent stem cells, wherein the HMC-EVs express at least one of the miRNAs in Table 10 at a lower level compared to UCB-MSC-EVs.

145. A population of HMC-EVs obtained by in vitro differentiation of pluripotent stem cells, wherein the HMC-EVs express at least one of the miRNAs in Table 11 at a higher level compared to BM-MSC-EVs.

146. A population of HMC-EVs obtained by in vitro differentiation of pluripotent stem cells, wherein the HMC-EVs express at least one of the miRNAs in Table 12 at a lower level compared to BM-MSC-EVs.

147. A population of HMC-EVs obtained by in vitro differentiation of pluripotent stem cells, wherein the HMC-EVs express at least one of the miRNAs in Table 13 at a higher level compared to AD-MSC-EVs.

148. A population of HMC-EVs obtained by in vitro differentiation of pluripotent stem cells, wherein the HMC-EVs express at least one of the miRNA in Table 14 at a lower level compared to AD-MSC-EVs.

149. A population of HMC-EVs obtained by in vitro differentiation of pluripotent stem cells, wherein the HMC-EVs express at least one of the proteins in Table 15 at a higher level compared to UCB-MSC-EVs.

150. A population of HMC-EVs obtained by in vitro differentiation of pluripotent stem cells, wherein the HMC-EVs express at least one of the proteins in Table 16 at a lower level compared to UCB-MSC-EVs.

151. A population of HMC-EVs obtained by in vitro differentiation of pluripotent stem cells, wherein the HMC-EVs express at least one of the proteins in Table 17 at a higher level compared to (BM-MSC-EVs.

152. A population of HMC-EVs obtained by in vitro differentiation of pluripotent stem cells, wherein the HMC-EVs express at least one of the proteins in Table 18 at a lower level compared to BM-MSC-EVs.

153. A population of HMC-EVs obtained by in vitro differentiation of pluripotent stem cells, wherein the HMC-EVs express at least one of the proteins in Table 19 at a higher level compared to AD-MSC-EVs.

154. A population of HMC-EVs obtained by in vitro differentiation of pluripotent stem cells, wherein the HMC-EVs express at least one of the proteins in Table 20 at a lower level compared to AD-MSC-EVs.

155. A population of HMC-EVs obtained by in vitro differentiation of pluripotent stem cells, wherein the HMC-EVs express at least one of the miRNAs selected from the group consisting of hsa-miR-125b-5p, hsa-miR-181a-5p, hsa-miR-199b-5p, hsa-miR-21-5p, hsa-miR-23a-3p, hsa-miR-125a-5p, hsa-miR-106a-5p+hsa-miR-17-5p and hsa-miR-221-3p at a higher level compared to BM-MSC-EVs, UCB-MSC-EVs, and/or AD-MSC-EVs.

156. A population of HMC-EVs obtained by in vitro differentiation of pluripotent stem cells, wherein the HMC-EVs express at least one of the proteins selected from the group consisting of ALDOC, ANXA5, APBB2, BASP1, CAV1, CD81, CD99, CKM, EPB41L3, FDPS, GNAQ, GNG12, GP9, H2AC20, H2AC21, H3-3A, H3-7, H4-16, HLA-A, ITGA2, KPNA2, KRAS, KRT4, LRRC59, MAMDC2, MARCKSL1, MDGA1, MERTK, MFGE8, MMP14, MVP, PCDH1, PDGFRB, PDIA3, RPL13, RPS18, RPS3A, RPS4X, SDCBP, SLC2A1, SLC3A2, TAGLN2, TNC, TSPAN14, TSPAN33, TSPAN9, TTYH3, UCHL1, VAT1, YWHAB, and YWHAQ at a higher level compared to BM-MSC-EVs, UCB-MSC-EVs, and/or AD-MSC-EVs.

157. A population of HMC-EVs obtained by in vitro differentiation of pluripotent stem cells, wherein the HMC-EVs express at least one of the proteins selected from the group consisting of ADGRG6, AGRN, ANXA6, APOC4, ARHGAP1, ARGHDIA, ARL8A, ARPC5, B2M, BBS1, BLVRA, BST1, CA2, CCN2, CCNB3, CD34, CD36, CD47, CORO1A, DTD1, EEF1D, EEF1G, ENG, ESD, GNAI2, GNB1, H1-3, H2BC15, HIP1, KIF11, LAMP1, LAP3, LGALS1, LTBP3, MAPK3, MARCKS, MBTD1, MDH1, MOB1B, MYL12B, MYO1F, MYO3A, NIBAN2, PEBP1, PF4, PGAP1, PLOD1, PPP2R1A, PRSS23, PXDN, RALA, RAP2A, RPS13, RPS3, RPSA, S100A11, SLC44A1, SLC44A2, SLTM, SMG1, SPARC, SRSF8, STRADB, STX11, STXBP2, TGM2, TPP1, TPTE2, TRIM5, TRPM2, TUBA8, TUBB3, VCAN, YWHAE, and ZFN607 at a higher level compared to BM-MSC-EVs, UCB-MSC-EVs, and/or AD-MSC-EVs.

158. A population of HMC-EVs obtained by in vitro differentiation of pluripotent stem cells, wherein the HMC-EVs express at least one of the proteins selected from the group consisting of ADIPOQ, CAT, CEP290, IGLV6-57, TAS2R33, and TMEM198 at a lower level compared to BM-MSC-EVs, UCB-MSC-EVs, and/or AD-MSC-EVs.

159. A population of HMC-EVs obtained by in vitro differentiation of pluripotent stem cells, wherein the HMC-EVs express at least one of the proteins selected from the group consisting of AKAP9, ALB, ALOX5, APLP2, CD109, CDSN, CHST9, ERC1, F11, ARMCX5, LAMB4, LRRTM2, LTF, MSH6, OAF, OLFML3, PAK6, RGS14, SEMA7A, SURF1, and TRIM4 at a lower level compared to BM-MSC-EVs, UCB-MSC-EVs, and/or AD-MSC-EVs.

160. A population of HMC-EVs obtained by in vitro differentiation of pluripotent stem cells, wherein the HMC-EVs express at least one of the miRNA in Table 21 at a higher level compared to the HMCs.

161. A population of HMC-EVs obtained by in vitro differentiation of pluripotent stem cells, wherein the HMC-EVs express at least one of the miRNA in Table 22 at a lower level compared to the HMCs.

162. A pharmaceutical composition comprising the HMC-EVs of any one of claims 143-161, and a pharmaceutically acceptable carrier.

163. A method of determining neurite outgrowth of an HMC population comprising:
(a) preparing a mixed neuronal culture from an isolated cerebral cortex;
(b) plating the HMC population on a permeable membrane;
(c) applying strain on the mixed neuronal culture;
(d) overlaying the strained mixed neuronal culture with the permeable membrane of step (b); and
(e) measuring neurite outgrowth of the mixed neuronal culture.

164. The method of claim 163, further determining gene expression of the mixed neuronal culture in the presence and absence of the HMC population.

165. The method of claim 163, wherein the strain is a physical scratch made in the mixed neuronal culture.

166. The method of claim 163, wherein the strain is vacuum pressure and positive air pressure applied to the mixed neuronal culture.

167. The method of claim 163, wherein the strain is applied at 15% to 0% stretching oscillations.
